(19) United States
(12) Patent Application Publication
(10) Pub. No.: US 2025/0311618 A1
(43) Pub. Date: Oct. 2, 2025

(54) AROMATIC AMINE COMPOUND, ORGANIC ELECTROLUMINESCENT DEVICE, AND ELECTRONIC APPARATUS

(71) Applicant: Shaanxi Lighte Optoelectronics Material Co., Ltd., Xi'an (CN)

(72) Inventors: Fumin YUE, Xi'an (CN); Youngkook KIM, Xi'an (CN); Linwei ZHANG, Xi'an (CN)

(21) Appl. No.: 18/863,178

(22) PCT Filed: Aug. 25, 2023
(86) PCT No.: PCT/CN2023/115097

§ 371 Date: Nov. 5, 2024

(30) Foreign Application Priority Data
Apr. 28, 2023 (CN) 202310487343.4
Jun. 1, 2023 (CN) 202310646272.8

(51) Int. Cl. H10K 85/60; C07B 59/00; C07D 307/91; C07D 333/76; C07D 405/12; C07D 405/14; C07D 409/12; H10K 50/15

(52) U.S. Cl. CPC H10K 85/633; C07B 59/002; C07D 307/91; C07D 333/76; C07D 405/12; C07D 405/14; C07D 409/12; H10K 85/636; H10K 50/156; H10K 85/615; H10K 85/622; H10K 85/626; H10K 85/6572; H10K 85/6574; H10K 85/6576

(57) ABSTRACT

The present disclosure relates to the field of organic electroluminescent materials. An aromatic amine compound and an organic electroluminescent device and an electronic apparatus containing the same are provided. In the aromatic amine compound of the present disclosure, aryl is connected to the 1-position of dibenzofuran (or dibenzothiophene) and arylamine is connected to the 2-position. This method ensures that a benzene ring connected to a nitrogen atom contains ortho substituted aryl, which can improve the mobility of molecules. By changing the conjugated group of substituents to adjust the injection and transport balance of holes between a hole transport layer and an luminescent layer, the luminous efficiency of the device is improved.

### CROSS REFERENCE TO RELATED APPLICATIONS

The present application claims priority to Chinese Patent Application No. CN202310487343.4, filed on Apr. 28, 2023, and Chinese Patent Application No. CN202310646272.8, filed on Jun. 1, 2023, the entire contents of which are incorporated herein by reference.

### TECHNICAL FIELD

The present disclosure relates to the technical field of organic electroluminescent materials, in particular to an aromatic amine compound, an organic electroluminescent device and an electronic apparatus containing the same.

### BACKGROUND

With the development of electronic technology and the progress of material science, the application scope of electronic components for achieving electroluminescence or photoelectric conversion is increasingly wide. An organic electroluminescent device (OLED) usually includes a cathode and an anode arranged oppositely, and a functional layer arranged between the cathode and the anode. The functional layer is composed of a plurality of organic or inorganic film layers, and generally includes an organic electroluminescent layer, a hole transport layer, an electron transport layer, etc. When a voltage is applied to the cathode and the anode, the two electrodes generate an electric field. Under the action of the electric field, electrons on the cathode side move to an electroluminescent layer, holes on the anode side also move to the electroluminescent layer, the electrons and the holes combine in the electroluminescent layer to form excitons, and the excitons are in an excited state to release energy to the outside, so that the electroluminescent layer emits light to the outside.

In existing organic electroluminescent devices, the main problems are embodied in lifetime and efficiency. With the increasing size of displays, the driving voltage also increases. Research on improving the performance of OLED electroluminescent devices includes reducing the driving voltage for the devices, improving the luminous efficiency of the devices, and prolonging the lifetime of the devices. To improve the performance of OLED devices, a multi-layer sandwich structure is usually used in the design of device structures, that is, an anode, a cathode, and an organic functional layer together form a complete device. Hole transport materials are materials that can accept positively charged hole carriers and effectively transfer the hole carriers or block electron transport. They usually have high hole mobility and low ionization potential, and are a very important part of organic electroluminescent devices. It is necessary to continue developing novel hole transport materials, so as to further improve the performance of organic electroluminescent devices.

### SUMMARY

In view of the above problems existing in the prior art, the objective of the present disclosure is to provide an aromatic amine compound and an organic electroluminescent device containing the same, and an electronic apparatus. The aromatic amine compound is used in the organic electroluminescent device to improve the performance of the device.

According to a first aspect of the present disclosure, an aromatic amine compound is provided. The aromatic amine compound has a structure represented by formula 1:

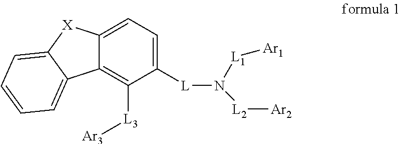

where X is selected from O or S;
L is selected from a single bond and substituted or unsubstituted phenylene;
L₁ and L₂ are the same or different, and are each independently selected from a single bond, substituted or unsubstituted arylene with 6 to 15 carbon atoms, and substituted or unsubstituted heteroarylene with 12 to 18 carbon atoms;
L₃ is selected from a single bond, substituted or unsubstituted phenylene, and substituted or unsubstituted naphthylene;
the substituent(s) in L, L₁, L₂, and L₃ are the same or different, and are each independently selected from deuterium, cyano, a halogen group, alkyl with 1 to 4 carbon atoms, haloalkyl with 1 to 4 carbon atoms, deuterated alkyl with 1 to 4 carbon atoms and phenyl;
Ar₁ and Ar₂ are the same or different, and are each independently selected from substituted or unsubstituted aryl with 6 to 40 carbon atoms, and substituted or unsubstituted heteroaryl with 5 to 40 carbon atoms;
Ar₃ is selected from substituted or unsubstituted biphenyl, substituted or unsubstituted terphenyl, substituted or unsubstituted naphthyl, substituted or unsubstituted phenanthryl, and substituted or unsubstituted triphenylene;
the substituent(s) in Ar₃ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms and phenyl; and
the substituent(s) in Ar₁ and Ar₂ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms, alkoxyl with 1 to 10 carbon atoms, trialkylsilyl with 3 to 12 carbon atoms, triphenylsilyl, aryl with 6 to 20 carbon atoms, deuterated aryl with 6 to 20 carbon atoms, heteroaryl with 3 to 20 carbon atoms, cycloalkyl with 3 to 10 carbon atoms, alkylthiol with 1 to 10 carbon atoms, aryloxyl with 6 to 20 carbon atoms, and arylthiol with 6 to 20 carbon atoms; optionally, any two adjacent substituents form a 5- to 15-membered ring.

According to a second aspect of the present disclosure, an organic electroluminescent device is provided, including an anode and a cathode arranged oppositely, and a functional layer arranged between the anode and the cathode, where the functional layer contains the aromatic amine compound described above.

According to a third aspect of the present disclosure, an electronic apparatus is provided, including the organic electroluminescent device described in the second aspect.

In the compound of the present disclosure, aryl is connected to the 1-position of dibenzofuran (or dibenzothiophene), and arylamine is connected to the 2-position of dibenzofuran (or dibenzothiophene). This method ensures that a benzene ring connected to a nitrogen atom contains two ortho substituted aryl, which can improve the mobility of molecules. By changing the conjugated group of substituents to adjust the injection and transport balance of holes between the hole transport layer and the luminescent layer, the voltage for the device is reduced and the luminous efficiency of the device is improved. Meanwhile, selecting several specific groups for the aryl at the 1-position of dibenzofuran (or dibenzothiophene) can increase the glass transition temperature Tg of the material, thereby ensuring that the device made of this material has a relatively long luminescence lifetime.

### BRIEF DESCRIPTION OF THE DRAWINGS

The accompanying drawings are used to provide a further understanding of the present disclosure, constitute a part of the description, and are used for interpreting the present disclosure together with the following specific embodiments, rather than limiting the present disclosure.

FIG. 1 is a schematic structural diagram of an organic electroluminescent device in an embodiment of the present disclosure.

FIG. 2 is a schematic structural diagram of an electronic apparatus in an embodiment of the present disclosure.

### DRAWING REFERENCE SIGNS

100. Anode
200. Cathode
300. Functional layer
310. Hole injection layer
321. Hole transport layer
322. Hole adjustment layer
330. Organic luminescent layer
340. Hole blocking layer
350. Electron transport layer
360. Electron injection layer
320. Hole transport area
400. Electronic apparatus

### DETAILED DESCRIPTION

Exemplary embodiments are now described more comprehensively with reference to the accompanying drawings. However, the exemplary embodiments can be implemented in various forms, and should not be construed as being limited to the examples set forth herein. On the contrary, these embodiments are provided to make the present disclosure more comprehensive and complete, and fully convey the concept of the exemplary embodiments to those skilled in the art. The described features, structures, or characteristics can be combined in one or more embodiments in any suitable manner. In the following description, many specific details are provided to give a sufficient understanding of the embodiments of the present disclosure.

In a first aspect, the present disclosure provides an aromatic amine compound. The aromatic amine compound has a structure represented by formula 1:

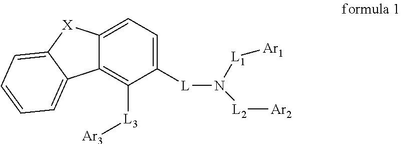

where X is selected from O or S;
L is selected from a single bond and substituted or unsubstituted phenylene;
L₁ and L₂ are the same or different, and are each independently selected from a single bond, substituted or unsubstituted arylene with 6 to 15 carbon atoms, and substituted or unsubstituted heteroarylene with 12 to 18 carbon atoms;
L₃ is selected from a single bond, substituted or unsubstituted phenylene, and substituted or unsubstituted naphthylene;
the substituent(s) in L, L₁, L₂, and L₃ are the same or different, and are each independently selected from deuterium, cyano, a halogen group, alkyl with 1 to 4 carbon atoms, haloalkyl with 1 to 4 carbon atoms, deuterated alkyl with 1 to 4 carbon atoms and phenyl
Ar₁ and Ar₂ are the same or different, and are each independently selected from substituted or unsubstituted aryl with 6 to 40 carbon atoms, and substituted or unsubstituted heteroaryl with 5 to 40 carbon atoms;
Ar₃ is selected from substituted or unsubstituted biphenyl, substituted or unsubstituted terphenyl, substituted or unsubstituted naphthyl, substituted or unsubstituted phenanthryl, and substituted or unsubstituted triphenylene;
the substituent(s) in Ar₃ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms and phenyl; and
the substituent(s) in Ar₁ and Ar₂ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms, alkoxyl with 1 to 10 carbon atoms, trialkylsilyl with 3 to 12 carbon atoms, triphenylsilyl, aryl with 6 to 20 carbon atoms, deuterated aryl with 6 to 20 carbon atoms, heteroaryl with 3 to 20 carbon atoms, cycloalkyl with 3 to 10 carbon atoms, alkylthiol with 1 to 10 carbon atoms, aryloxyl with 6 to 20 carbon atoms, and arylthiol with 6 to 20 carbon atoms; optionally, any two adjacent substituents form a 5- to 15-membered ring.

In the present disclosure, the terms “optional” and “optionally” mean that the events or environments described subsequently may or may not occur. For example, “optionally, in Ar₁ and Ar₂, any two adjacent substituents form a 5- to 15-membered ring” includes a situation where any two adjacent substituents form a ring, and a situation where any two adjacent substituents exist independently without forming a ring. “Any two adjacent” may include two substituents on the same atom, and may also include one substituent on each of two adjacent atoms, where when two substituents are on the same atom, the two substituents can form a saturated or unsaturated spiro ring together with the atom to which they are connected together; and when one substituent is on each of two adjacent atoms, the two substituents can be fused into a ring.

In the present disclosure, the ring system formed by n atoms is referred to as an n-membered ring. For example, phenyl is a 6-membered ring. The 5- to 15-membered ring refers to a cyclic group with 5 to 15 ring atoms. The ring atom may be carbon atom or heteroatoms selected from O, S, N, etc. The 5- to 15-membered ring is, for example, cyclopentane, cyclohexane, fluorene ring, or benzene ring.

In the present disclosure, the descriptions of “ . . . are. each independently”, “ . . . are respectively and independently”, and “each . . . is independently” are interchangeable and should be broadly understood, indicating that specific options expressed between the same symbols in different groups do not affect each other, or specific options expressed between the same symbols in the same group do not affect each other. For example,

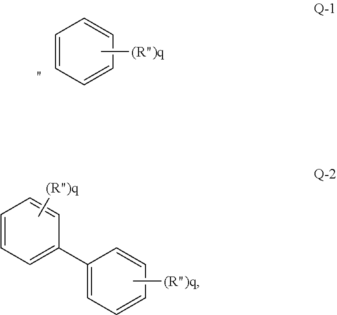

where each q is independently 0, 1, 2 or 3, and each R″ is independently selected from hydrogen, deuterium, fluorine, or chlorine″ means: formula Q−1 represents that the benzene ring has q substituents R″, each R″ may be the same or different, and the options of each R″ do not affect each other; and formula Q−2 represents that each benzene ring of biphenyl has q substituents R″, the numbers q of R″ substituents on the two benzene rings may be the same or different, each R″ may be the same or different, and the options of each R″ do not affect each other.

In the present disclosure, the term “substituted or unsubstituted” indicates that the functional group described behind the term may or may not have a substituent (hereinafter, for the convenience of description, the substituents are collectively referred to as Rc). For example, “substituted or unsubstituted aryl” indicates aryl with a substituent Rc or unsubstituted aryl. The substituent Rc may be, for example, deuterium, fluorine, cyano, heteroaryl, aryl, deuterated aryl, trialkylsilyl, alkyl, haloalkyl, deuterated alkyl, or cycloalkyl. The number of substituents may be 1 or more.

In the present disclosure, “a plurality of” refers to 2 or more, such as 2, 3, 4, 5, 6, etc.

In the present disclosure, the number of carbon atoms in a substituted or unsubstituted functional group refers to the total number of carbon atoms in the group and all substituents thereon. For example, if L₁ is substituted arylene with 12 carbon atoms, the number of all carbon atoms in the arylene and substituents thereon is 12.

The hydrogen atom in the compound structure of the present disclosure includes various isotopic atoms of hydrogen, such as hydrogen (H), deuterium (D), or tritium (T).

The “D” in the structural formula of the compound in the present disclosure represents deuteration.

In the present disclosure, the aryl refers to an optional functional group or substituent derived from an aromatic carbon ring. The aryl may be monocyclic aryl (such as phenyl) or polycyclic aryl. In other words, the aryl may be monocyclic aryl, fused-ring aryl, two or more monocyclic aryls conjugated by carbon-carbon bonds, monocyclic aryl and fused-ring aryl conjugated by carbon-carbon bonds, or two or more fused-ring aryls conjugated by carbon-carbon bonds. That is, unless otherwise specified, two or more aromatic groups conjugated by carbon-carbon bonds may also be considered as aryl in the present disclosure. The fused-ring aryl may include, for example, bicyclic fused aryl (such as naphthyl), tricyclic fused aryl (such as phenanthrenyl, fluorenyl, or anthracenyl). The aryl does not contain heteroatoms such as B, N, O, S, P, Se, and Si. Examples of the aryl include, but are not limited to, phenyl, naphthyl, fluorenyl, spirodifluorenyl

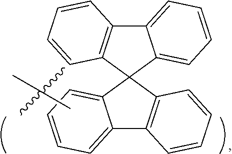

anthryl, phenanthryl, biphenyl, terphenyl, tetraphenyl, pentaphenyl, triphenylene

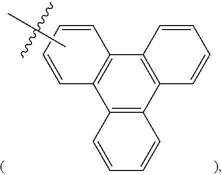

perylenyl, benzo[9,10]phenanthryl, pyrenyl, benzofluoranthenyl, chrysenyl, etc.

In the present disclosure, the arylene referred to refers to a divalent group formed by further loss of one or more hydrogen atoms from the aryl.

In the present disclosure, the terphenyl includes

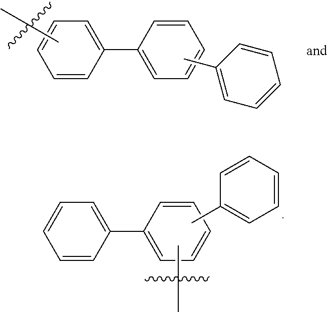

In the present disclosure, the number of carbon atoms in the substituted or unsubstituted aryl (arylene) may be 6, 8, 10, 12, 13, 14, 15, 16, 17, 18, 19, 20, 21, 22, 23, 24, 25, 30, 31, 32, 33, 34, 35, 36, 37, 38, 39, or 40. In some embodiments, the substituted or unsubstituted aryl is substituted or unsubstituted aryl with 6 to 30 carbon atoms. In other embodiments, the substituted or unsubstituted aryl is substituted or unsubstituted aryl with 6 to 25 carbon atoms. In other embodiments, the substituted or unsubstituted aryl is substituted or unsubstituted aryl with 6 to 18 carbon atoms. In other embodiments, the substituted or unsubstituted aryl is substituted or unsubstituted aryl with 6 to 15 carbon atoms.

In the present disclosure, the fluorenyl may be substituted by one or more substituents. When the fluorenyl is substituted, the substituted fluorenyl may be, but is not limited to,

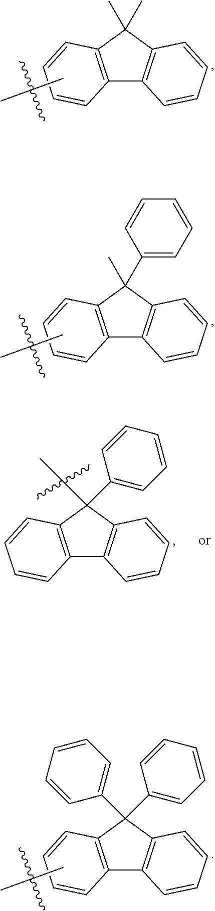

In the present disclosure, the aryl as substituents of Ar₁ and Ar₂ includes, but is not limited to, phenyl, naphthyl, phenanthryl, biphenyl, fluorenyl, dimethylfluorenyl, etc.

In the present disclosure, the heteroaryl refers to a monovalent aromatic ring containing 1, 2, 3, 4, 5, or 6 heteroatoms or derivatives thereof, and the heteroatoms may be one or more of B, O, N, P, Si, Se, and S. The heteroaryl may be monocyclic heteroaryl or polycyclic heteroaryl. In other words, the heteroaryl may be a system of a single aromatic ring or a system of multiple aromatic rings conjugated by carbon-carbon bonds, and any aromatic ring system is an aromatic monocyclic ring or an aromatic fused ring. For example, the heteroaryl may include, but is not limited to, thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridinopyrimidinyl, pyridinopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, N-phenylcarbazolyl, N-pyridylcarbazolyl, N-methylcarbazolyl, etc.

In the present disclosure, the heteroarylene referred to refers to a divalent or multivalent group formed by further loss of one or more hydrogen atoms from the heteroaryl.

In the present disclosure, the number of carbon atoms in the substituted or unsubstituted heteroaryl (heteroarylene) may be selected from 3, 4, 5, 6, 7, 8, 9, 10, 11, 12, 13, 14, 15, 16, 17, 18, 20, 21, 22, 23, 24, 25, 26, 27, 28, 29, 30, 31, 32, 33, 34, 35, 36, 37, 38, 39, or 40. In some embodiments, the substituted or unsubstituted heteroaryl is substituted or unsubstituted heteroaryl with totally 12 to 18 carbon atoms. In other embodiments, the substituted or unsubstituted heteroaryl is substituted or unsubstituted heteroaryl with totally 5 to 12 carbon atoms.

In the present disclosure, the heteroaryl as substituents of Ar₁ and Ar₂ includes, but is not limited to, pyridyl, carbazolyl, dibenzothienyl, dibenzofuranyl, benzoxazolyl, benzothiazolyl, and benzimidazolyl.

In the present disclosure, the substituted heteroaryl may indicate that one or more hydrogen atoms in the heteroaryl is substituted with groups such as a deuterium atom, a halogen group, cyano, aryl, heteroaryl, trialkylsilyl, alkyl, deuterated alkyl, cycloalkyl, haloalkyl, etc.

In the present disclosure, the alkyl with 1 to 10 carbon atoms may include linear alkyl with 1 to 10 carbon atoms and branched alkyl with 3 to 10 carbon atoms. The number of carbon atoms in the alkyl may be, for example, 1, 2, 3, 4, 5, 6, 7, 8, 9, or 10. Specific examples of the alkyl include, but are not limited to, methyl, ethyl, n-propyl, isopropyl, n-butyl, isobutyl, tert-butyl, n-pentyl, isopentyl, neopentyl, n-hexyl, etc.

In the present disclosure, the halogen group may be, for example, fluorine, chlorine, bromine, or iodine.

In the present disclosure, specific examples of the trialkylsilyl include, but are not limited to, trimethylsilyl, triethylsilyl, etc.

In the present disclosure, the haloalkyl refers to alkyl substituted with halogen, and specific examples of the haloalkyl include, but are not limited to, trifluoromethyl.

In the present disclosure, the deuterated alkyl refers to alkyl substituted with one or more deuterium atoms, and specific examples of the deuterated alkyl include, but are not limited to, trideuteromethyl.

In the present disclosure, the number of carbon atoms in the cycloalkyl with 3 to 10 carbon atoms may be, for example, 3, 4, 5, 6, 7, 8, or 10. Specific examples of the cycloalkyl include, but are not limited to, cyclopentyl, cyclohexyl, and adamantyl.

In the present disclosure, the number of carbon atoms in the deuterated alkyl with 1 to 10 carbon atoms is, for example, 1, 2, 3, 4, 5, 6, 7, 8, or 10. Specific examples of the deuterated alkyl include, but are not limited to, trideuteromethyl.

In the present disclosure, the number of carbon atoms in the haloalkyl with 1 to 10 carbon atoms is, for example, 1, 2, 3, 4, 5, 6, 7, 8, or 10. Specific examples of the haloalkyl include, but are not limited to, trifluoromethyl.

In the present disclosure,

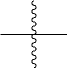

refers to a chemical bond interconnected with other groups.

In the present disclosure, the single bond

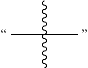

involved in a delocalized connecting bond and extending from a ring system indicates that one end of the connecting bond can be connected to any position through which the bond penetrates in the ring system, and the other end can be connected to the rest of the compound molecule. For example, as shown in the following formula (f), the naphthyl represented by formula (f) is connected to other positions of a molecule by two delocalized connecting bonds penetrating through double rings, which includes any possible connection as shown in formulas (f-1) to (f-10):

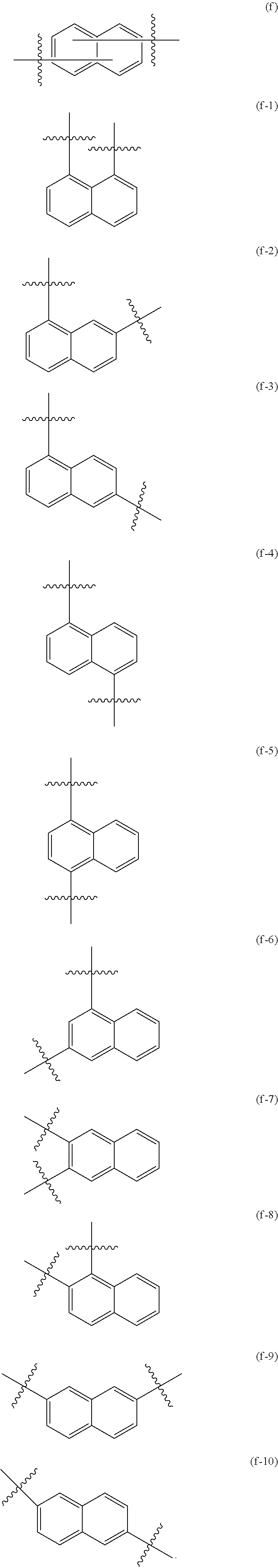

For another example, as shown in the following formula (X′), the dibenzofuranyl represented by formula (X′) is connected to other positions of a molecule by a delocalized connecting bond extending from the middle of a benzene ring on one side, which includes any possible connection as shown in formulas (X′-1) to (X′-4):

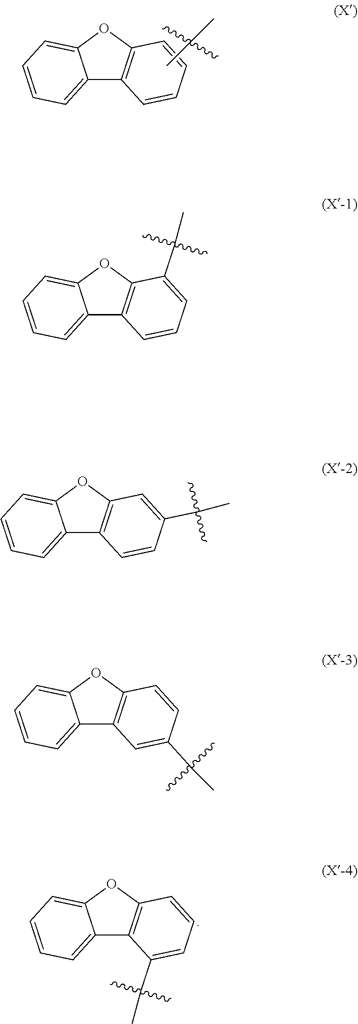

The delocalized substituent in the present disclosure refers to a substituent connected by a single bond extending from the center of a ring system, indicating that the substituent can be connected to any possible position in the ring system. For example, as shown in the following formula (Y), the substituent R′ represented by formula (Y) is connected to a quinoline ring through a delocalized connecting bond, which includes any possible connection as shown in formulas (Y-1) to (Y-7):

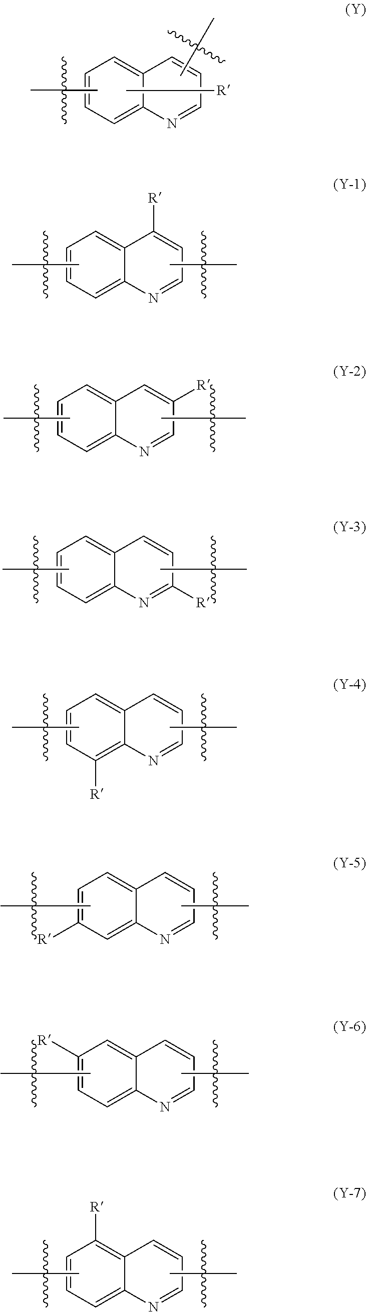

In some embodiments, L₁ and L₂ are each independently selected from a single bond, substituted or unsubstituted arylene with 6, 7, 8, 9, 10, 11, 12, 13, 14, or 15 carbon atoms, and substituted or unsubstituted heteroarylene with 12, 13, 14, 15, 16, 17, or 18 carbon atoms.

In some embodiments, L₁ and L₂ are the same or different, and are each independently selected from a single bond, substituted or unsubstituted phenylene, substituted or unsubstituted naphthylene, substituted or unsubstituted biphenylene, substituted or unsubstituted dibenzothienylene, substituted or unsubstituted dibenzofuranylene, and substituted or unsubstituted carbazolylene.

Optionally, the substituent(s) in L₁ and L₂ are the same or different, and are each independently selected from deuterium, fluorine, cyano, methyl, ethyl, isopropyl, tert-butyl, trifluoromethyl, trideuteromethyl, and phenyl.

In some embodiments, L is selected from a single bond and the following groups:

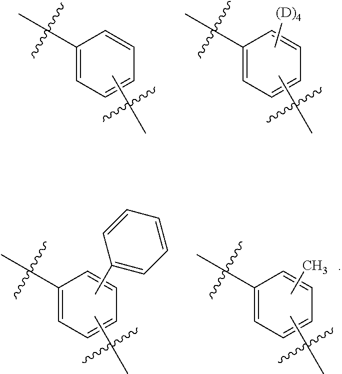

In some embodiments, L is selected from a single bond and the following groups:

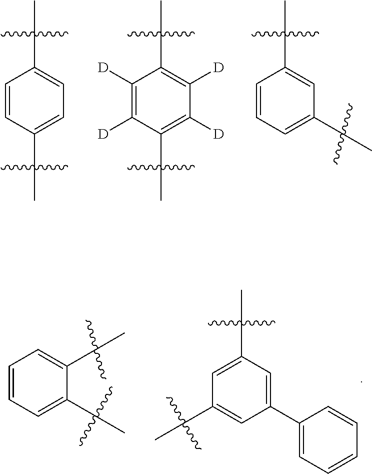

In some embodiments, L₁ and L₂ are the same or different, and are each independently selected from a single bond and the group consisting of the following groups:

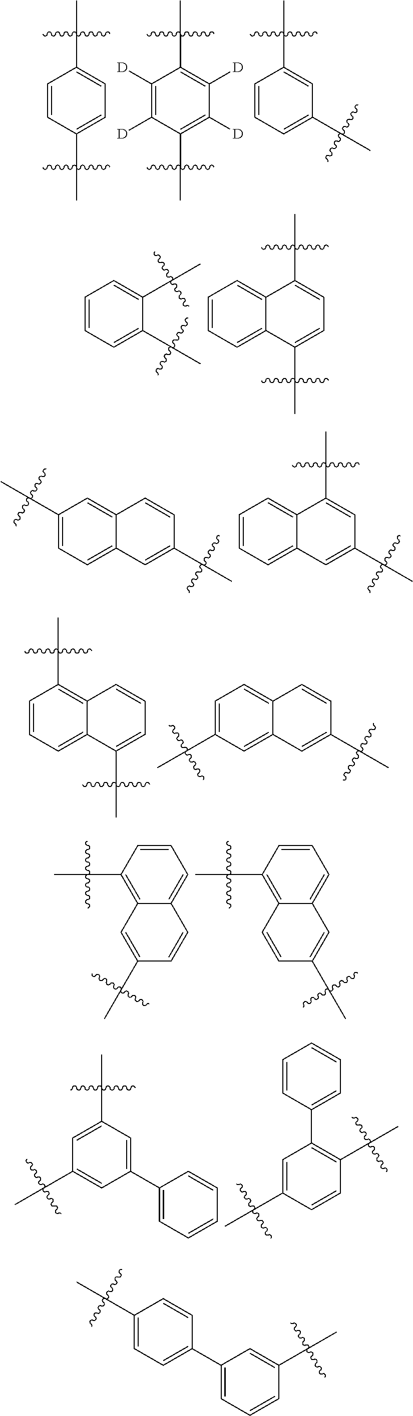

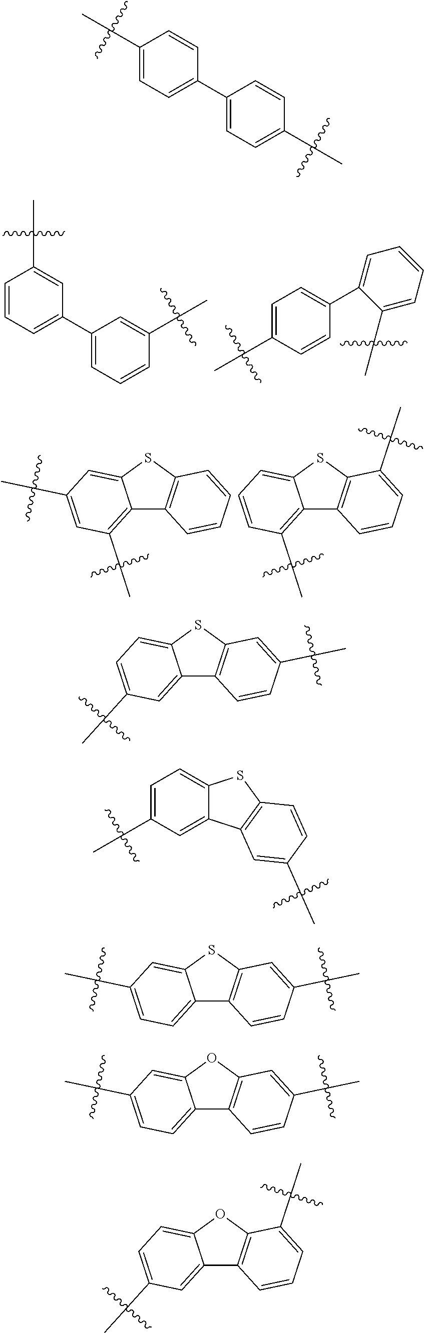

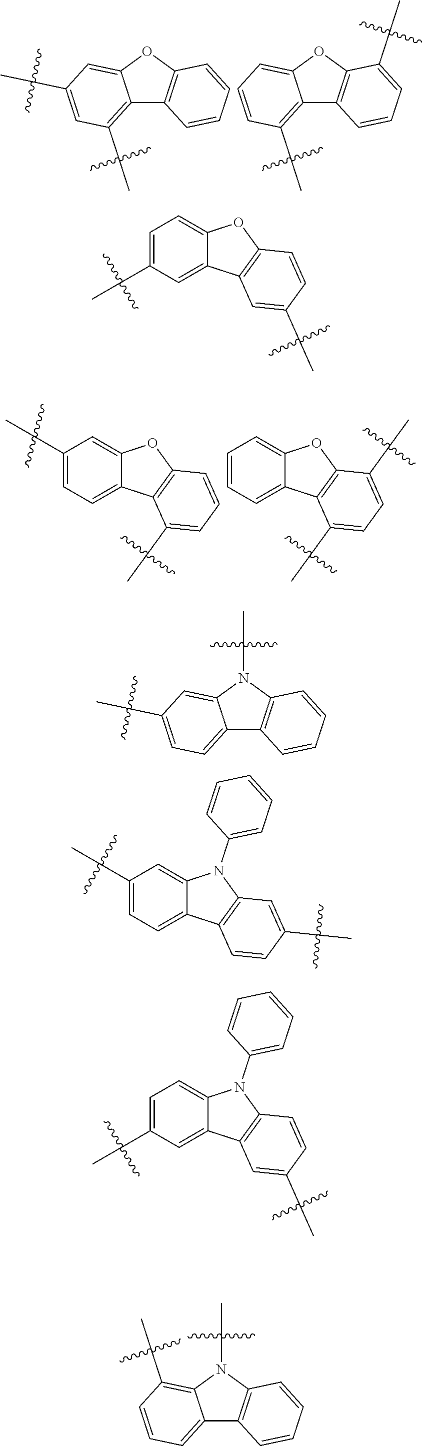

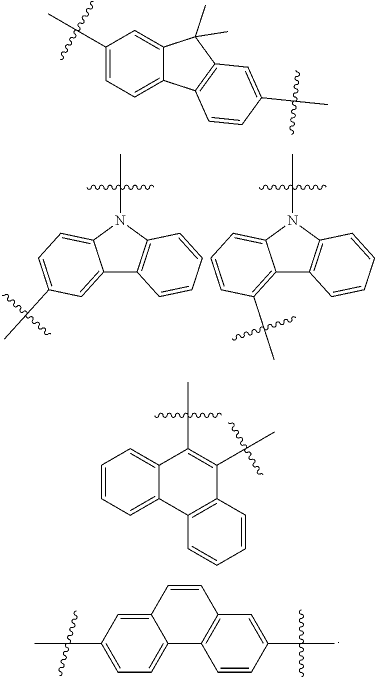

In some embodiments, L₃ is selected from a single bond and the following groups:

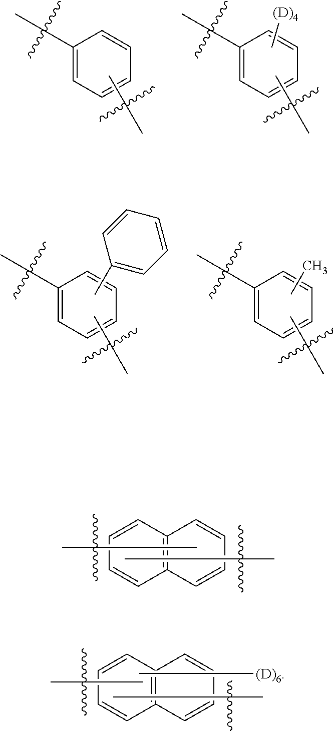

In some embodiments, L₃ is selected from a single bond and the following groups:

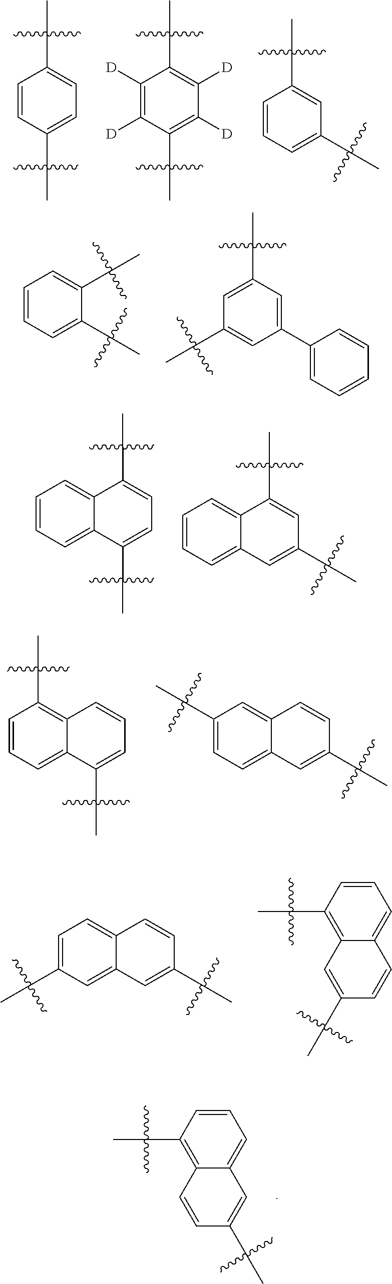

In some embodiments, Ar₃ is selected from the following groups:

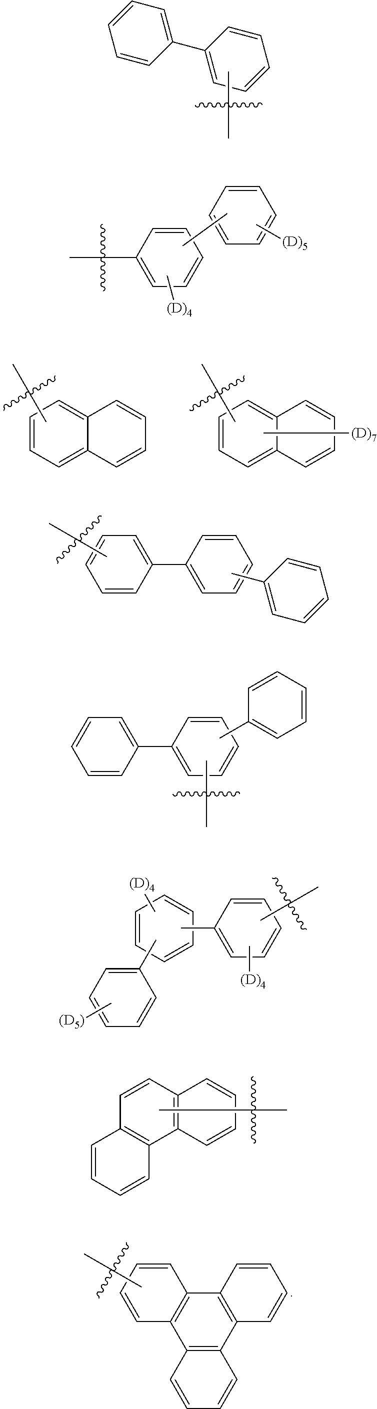

In some embodiments, Ar₃ is selected from the following groups:

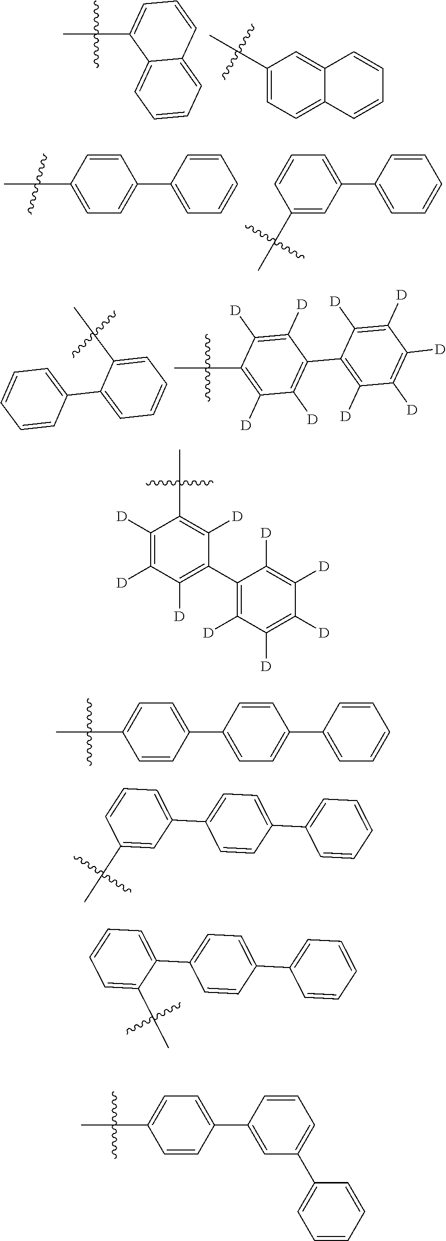

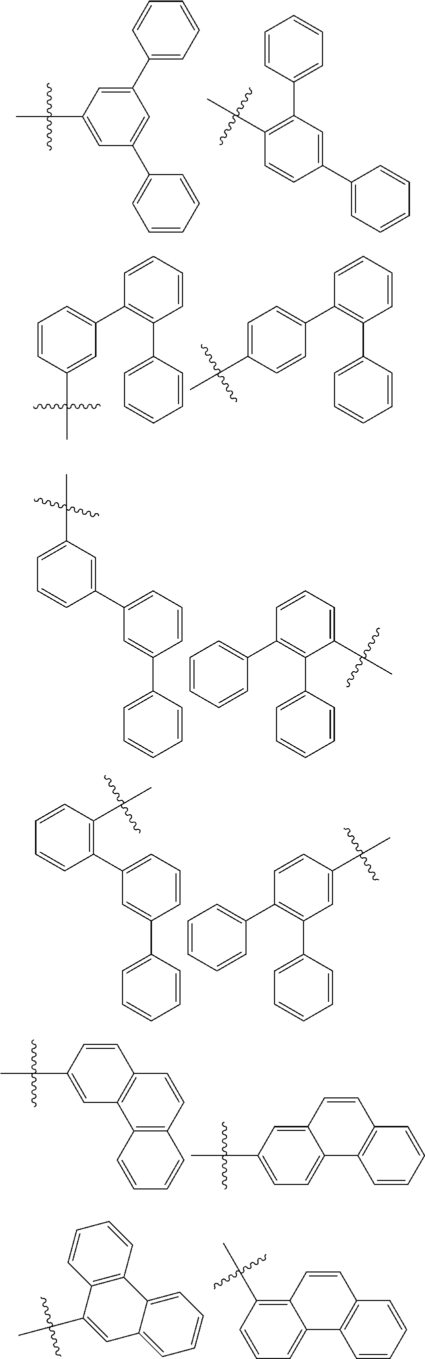

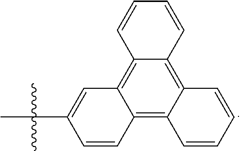 [SMILES: *c1ccc2c3ccccc3c3ccccc3c2c1]

In some embodiments,

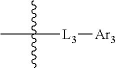

is selected from the group consisting of the following groups:

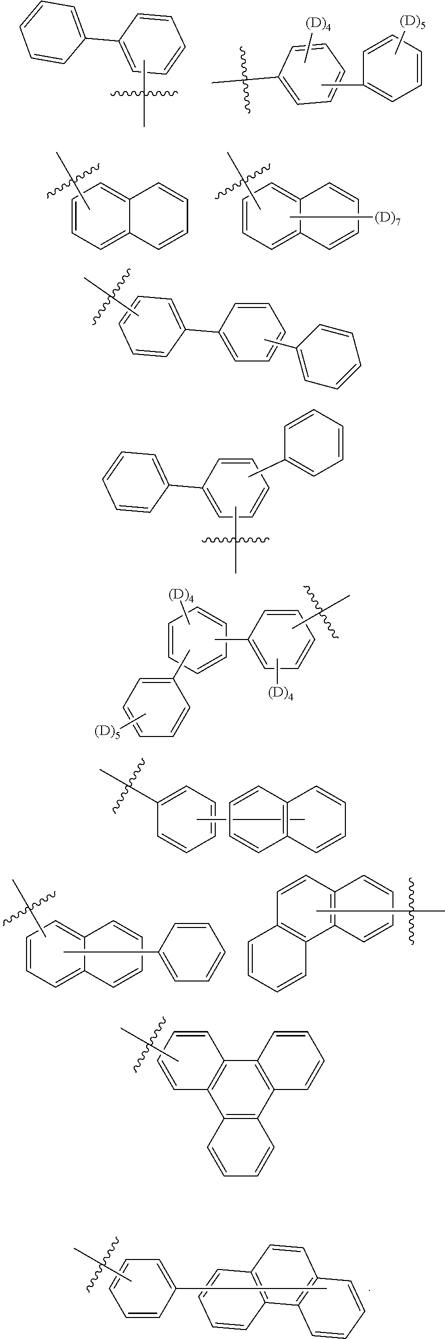

In some embodiments,

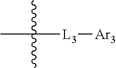

is selected from the group consisting of the following groups:

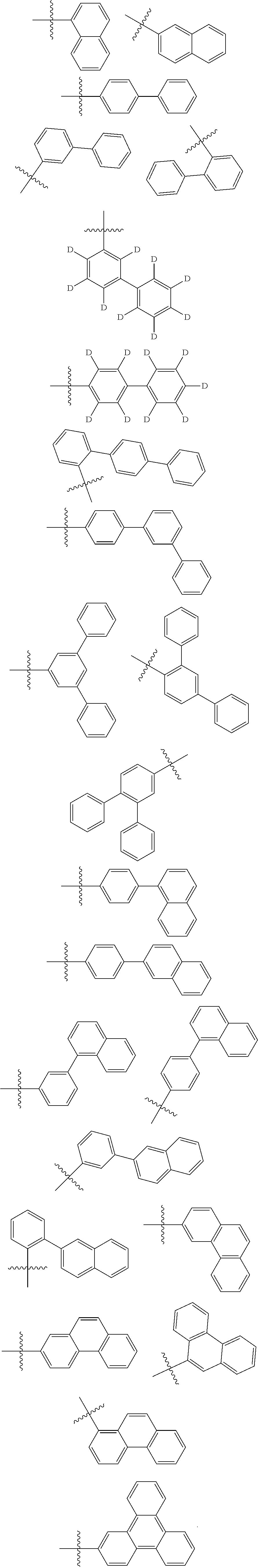

In the compound of the present disclosure, several specific substituents are connected to the 1-position of a parent nucleus dibenzofuran (or dibenzothiophene), so that the compound has a high degree of distortion in spatial structure, which ensures that the material has good amorphous property in a device, thereby improving film formation.

In some embodiments, Ar₁ and Ar₂ are the same or different, and are each independently selected from a substituted or unsubstituted group W, where the unsubstituted group W is selected from the group consisting of the following groups:

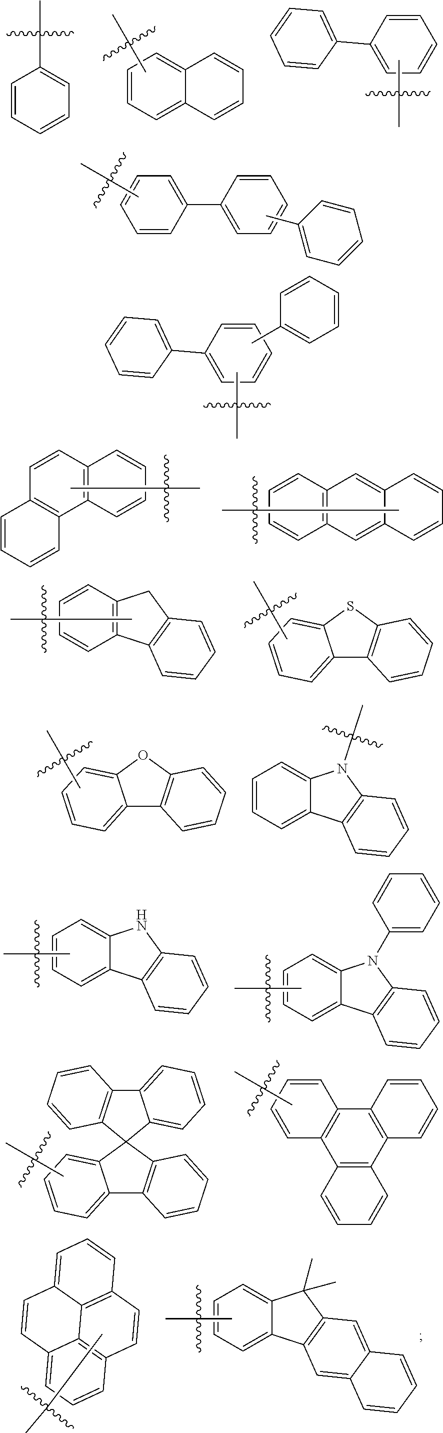

the substituted group W has one or more substituents, the substituent(s) on the substituted group W are each independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl, or carbazolyl, and when the number of substituents on the group W is greater than 1, the substituents are the same or different.

In some embodiments, Ar₁ and Ar₂ are each independently selected from substituted or unsubstituted aryl with 6, 7, 8, 9, 10, 11, 12, 13, 14, 15, 16, 17, 18, 19, 20, 21, 22, 23, 24, 25, 26, 27, 28, 29, or 30 carbon atoms, and substituted or unsubstituted heteroaryl with 3, 4, 5, 6, 7, 8, 9, 10, 12, 13, 14, 15, 16, 17, 18, 19, 20, 21, 22, 23, 24, 25, 26, 27, 28, 29, or 30 carbon atoms.

In some embodiments, Ar₁ and Ar₂ are the same or different, and are each independently selected from substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted benzofluorenyl, substituted or unsubstituted spirobifluorenyl, substituted or unsubstituted triphenylene, substituted or unsubstituted pyrenyl, substituted or unsubstituted perylenyl, substituted or unsubstituted dibenzothienyl, substituted or unsubstituted dibenzofuranyl, substituted or unsubstituted carbazolyl, substituted or unsubstituted benzothiazolyl, substituted or unsubstituted benzoxazolyl, and substituted or unsubstituted benzimidazolyl.

Optionally, the substituents in Ar₁ and Ar₂ are the same or different, and are each independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl, and carbazolyl; optionally, in Ar₁ and Ar₂, any two adjacent substituents form a benzene ring or a fluorene ring.

In some embodiments, Ar₁ and Ar₂ are the same or different, and are each independently selected from the group consisting of the following groups:

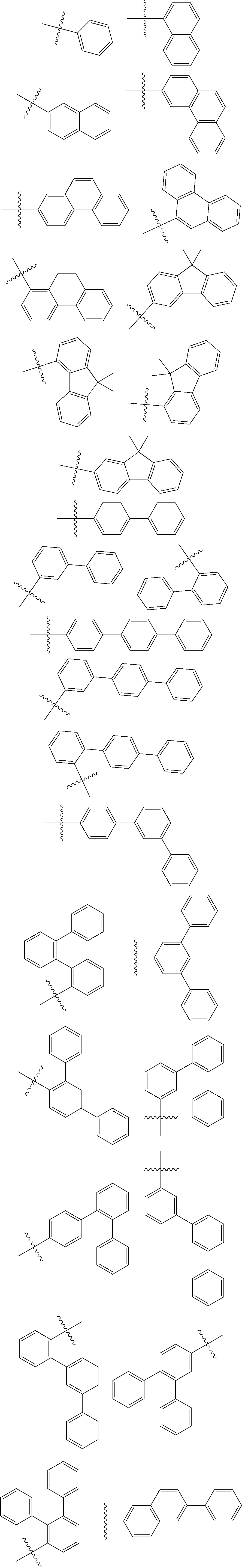

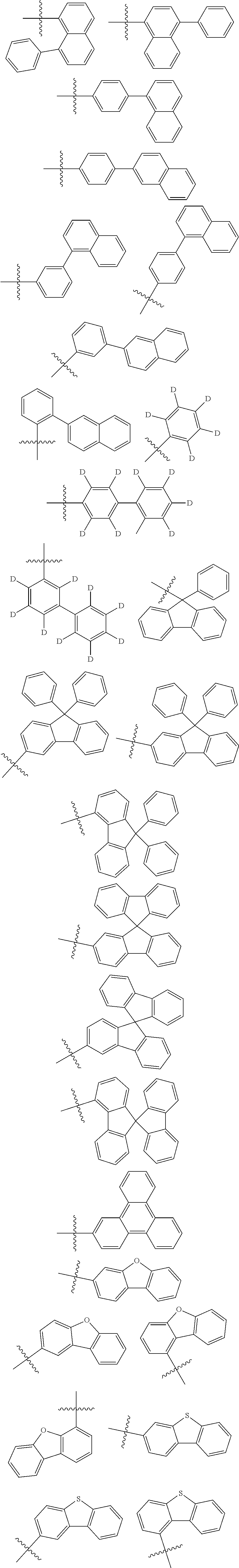

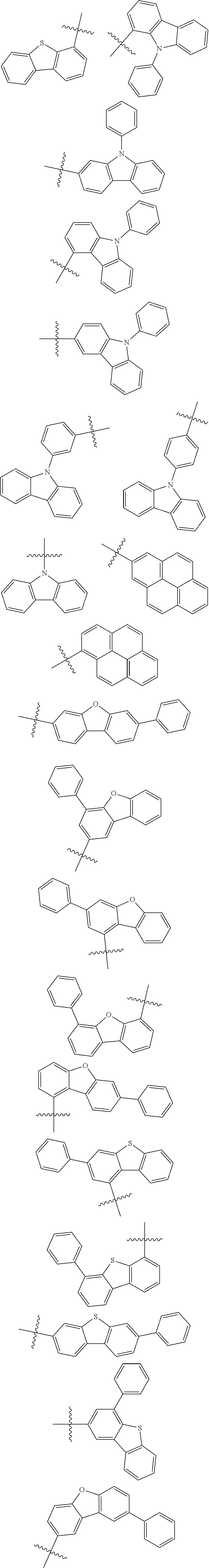

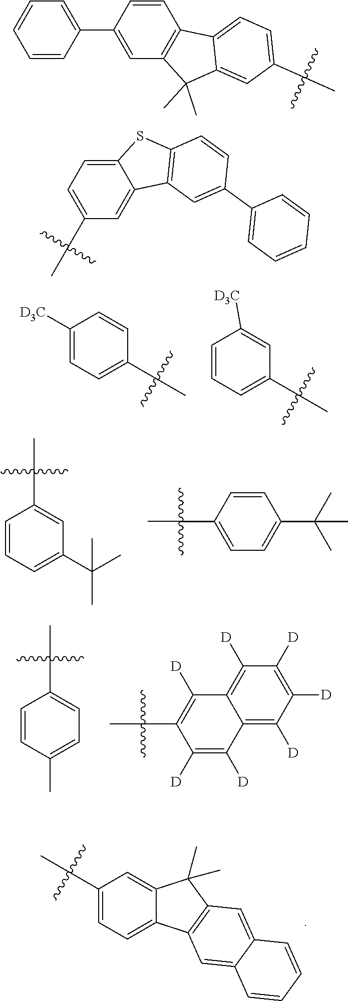

In some embodiments,

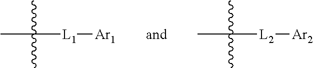

are each independently selected from the following groups:

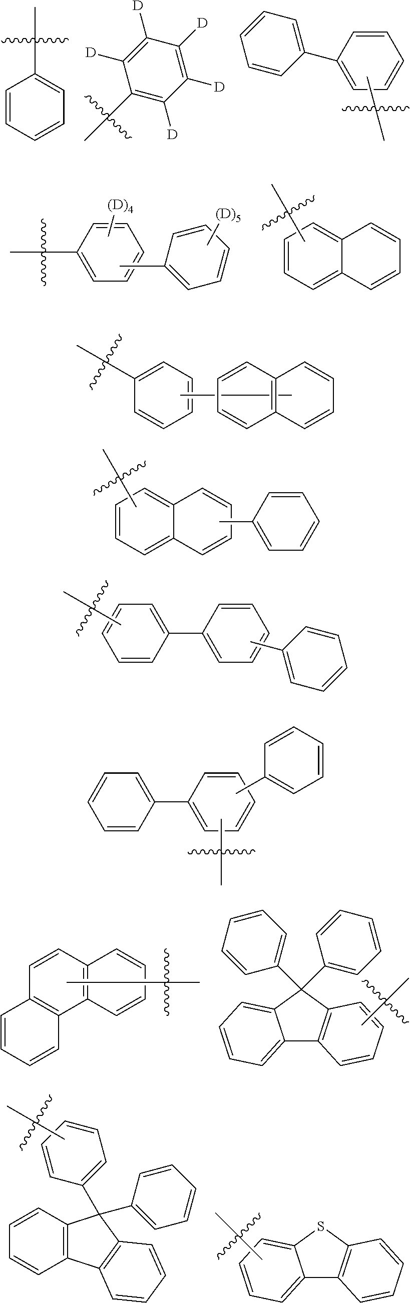

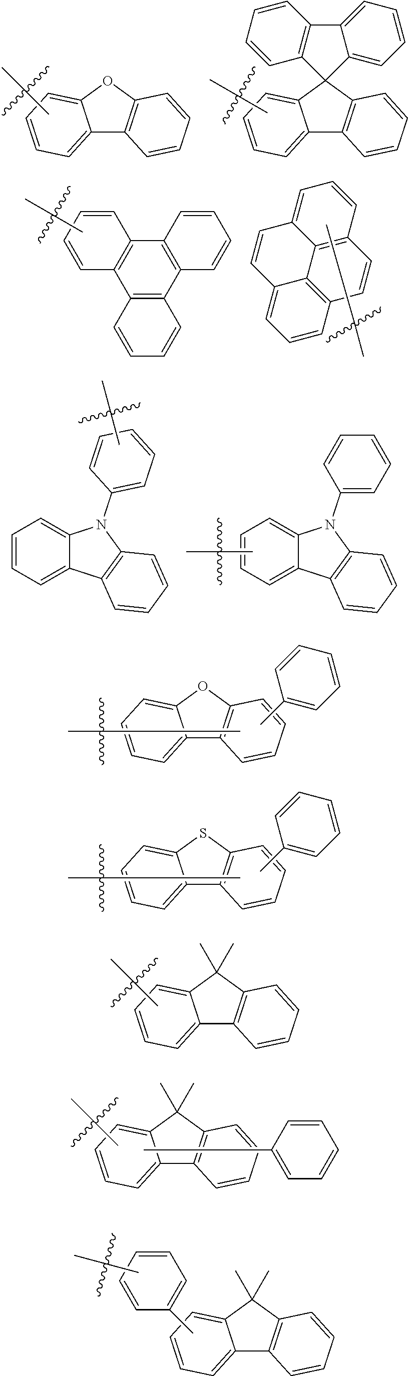

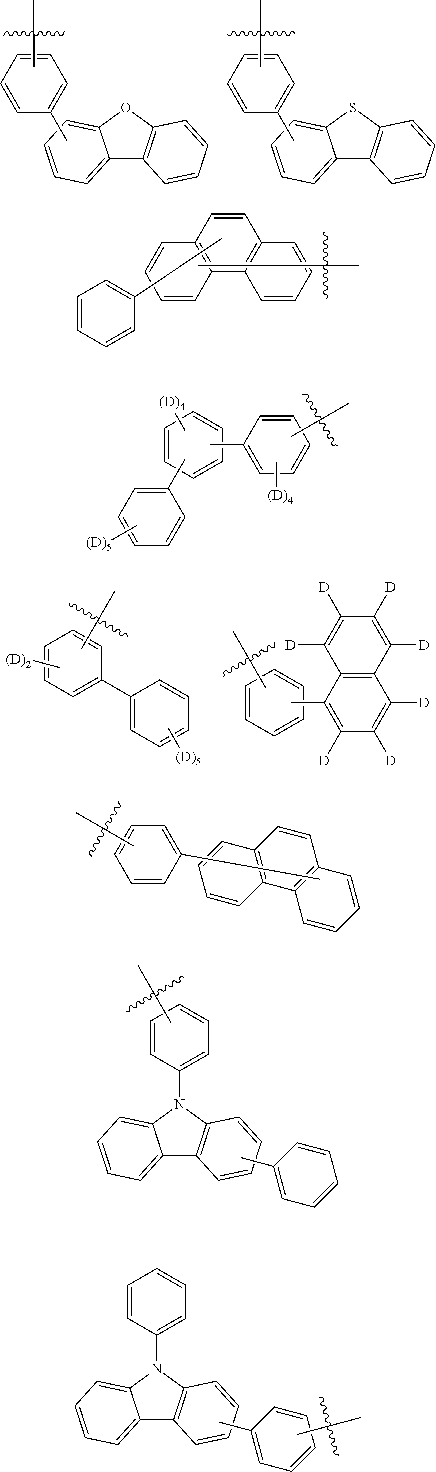

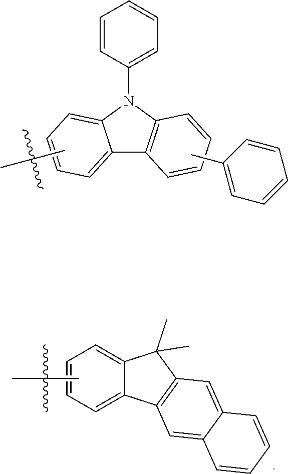

In some embodiments,

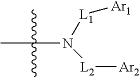

is selected from the following groups:

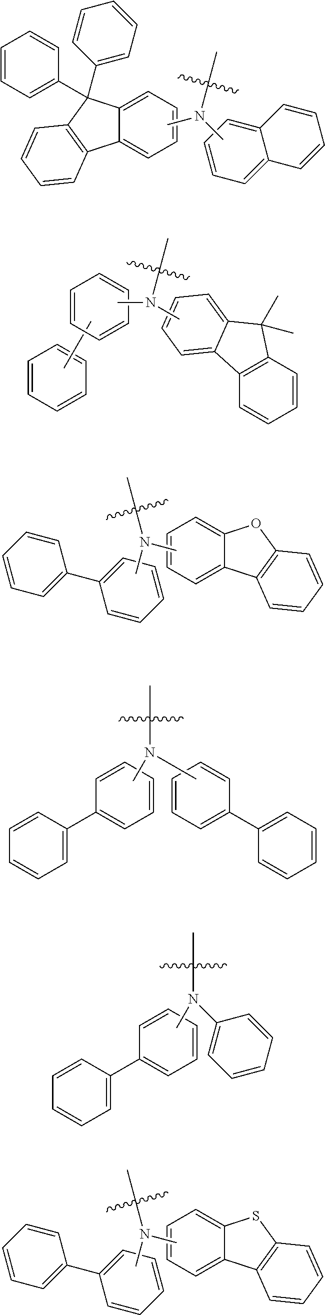

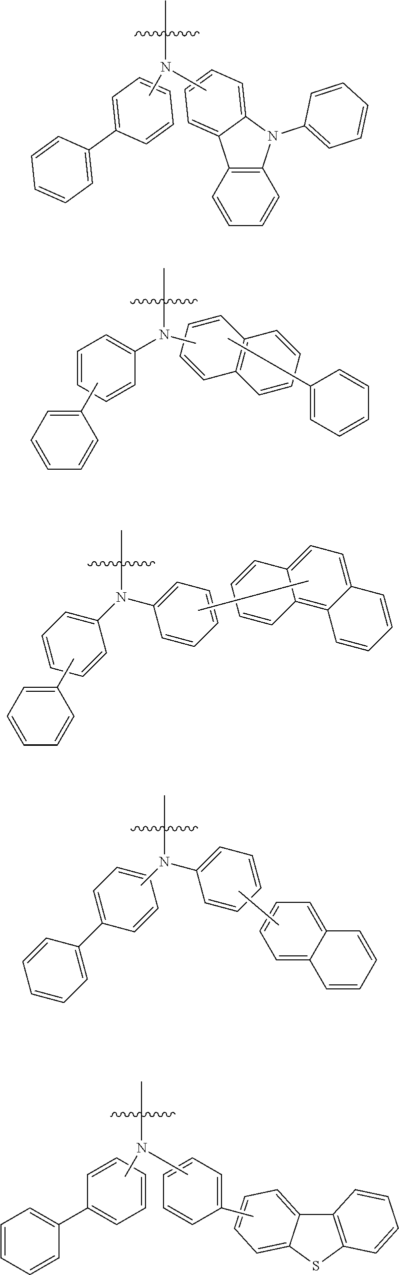

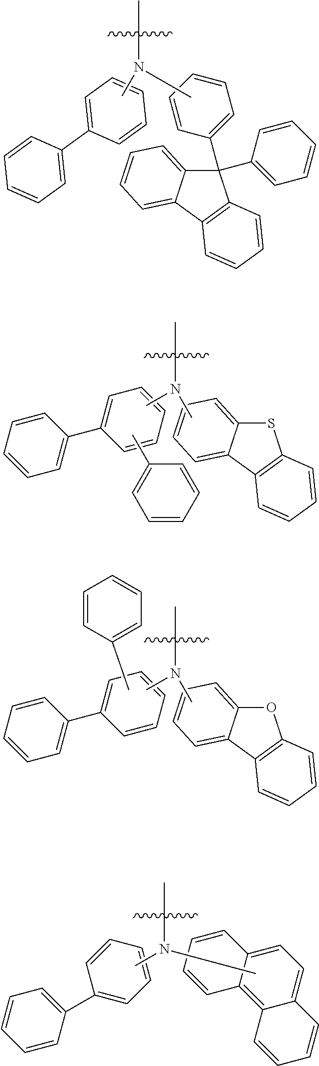

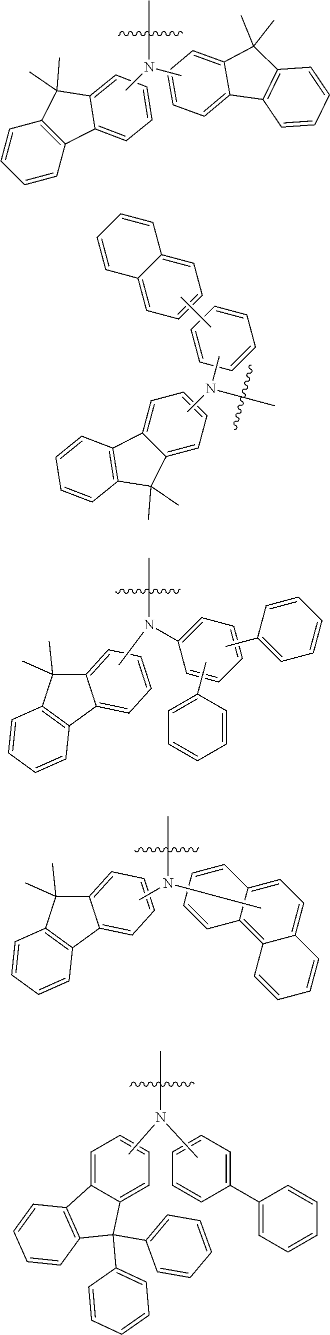

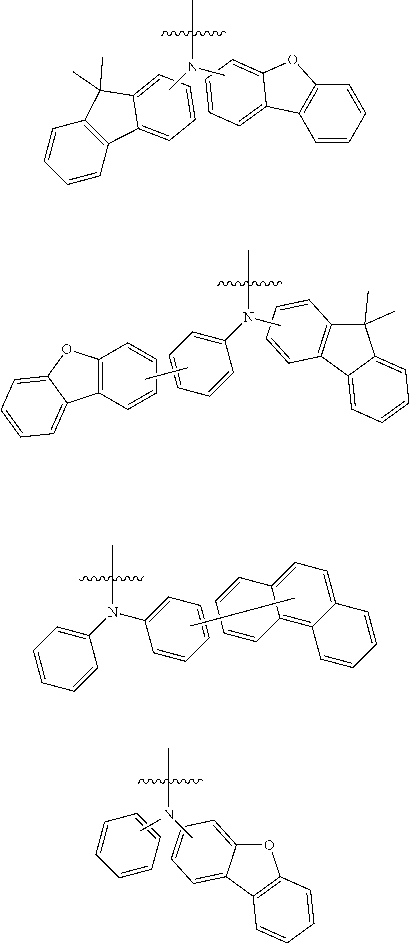

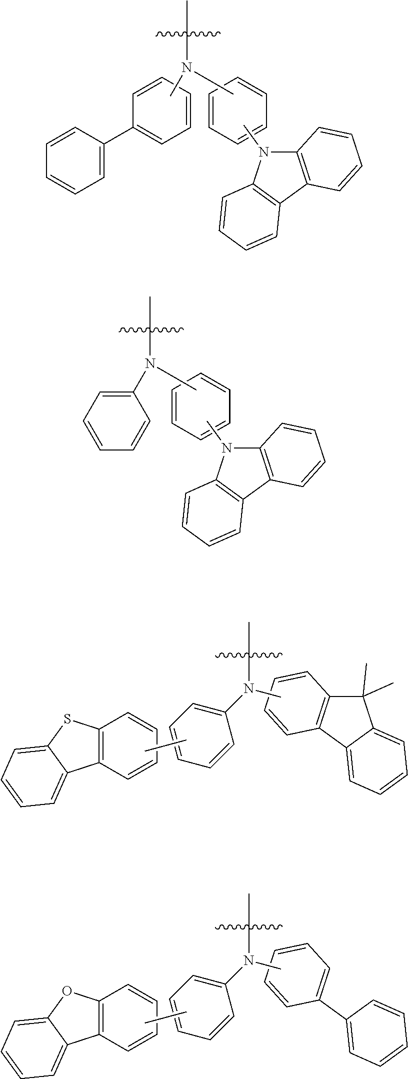

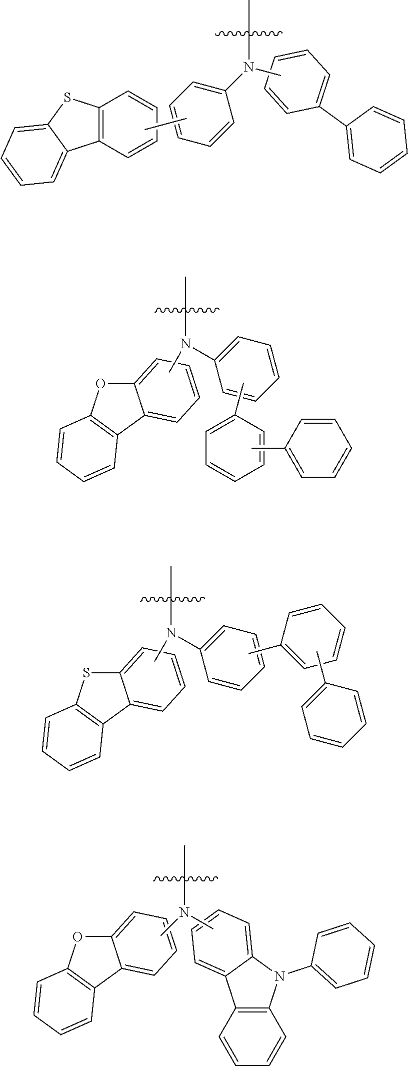

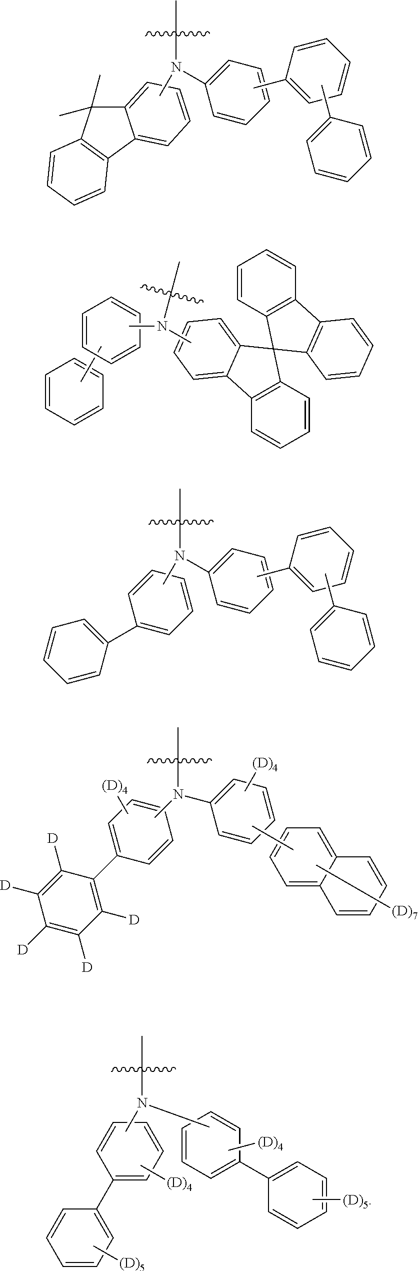

In some embodiments,

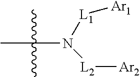

is selected from the following groups:

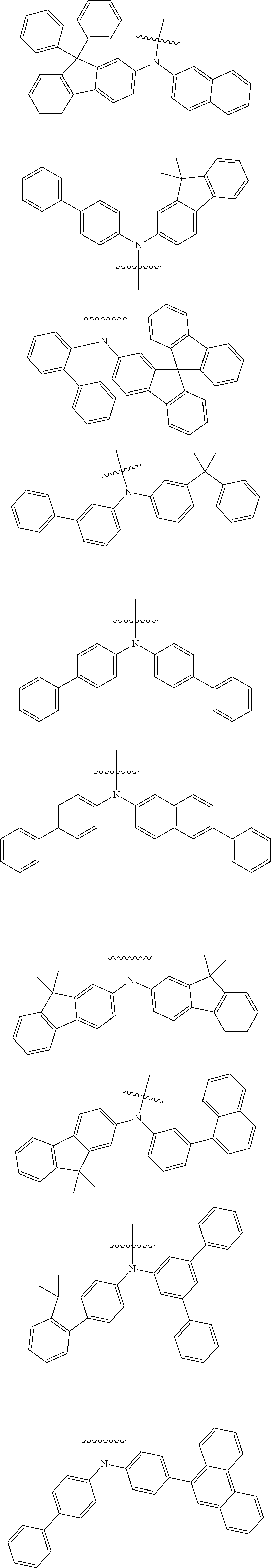

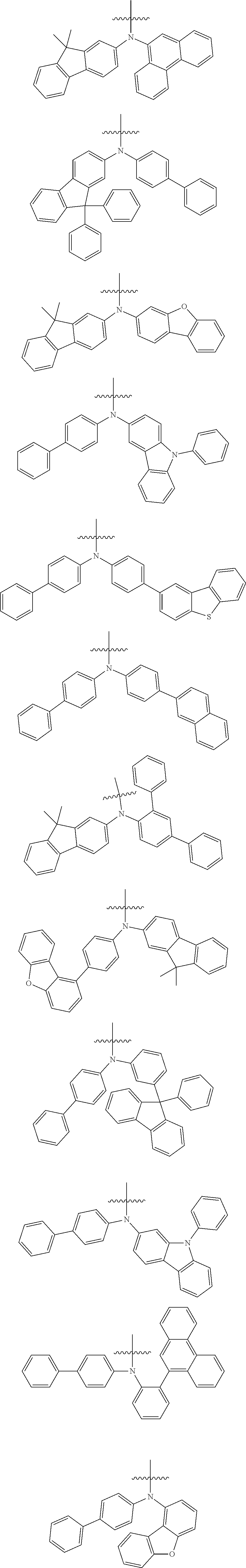

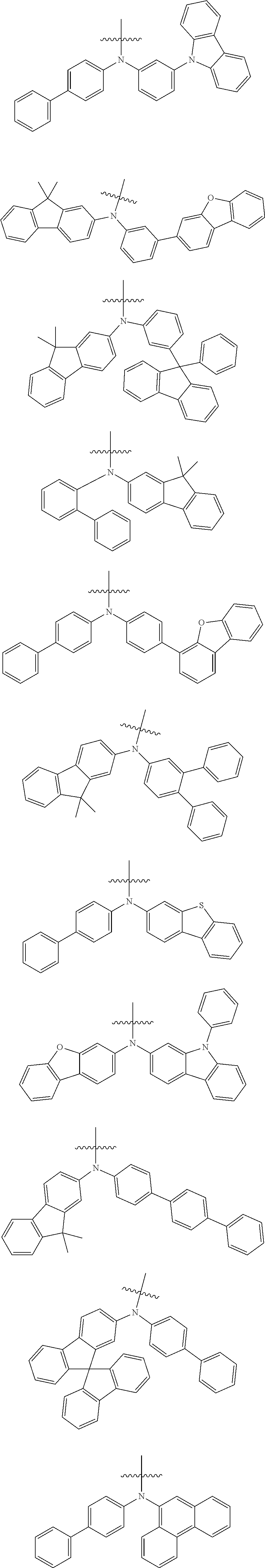

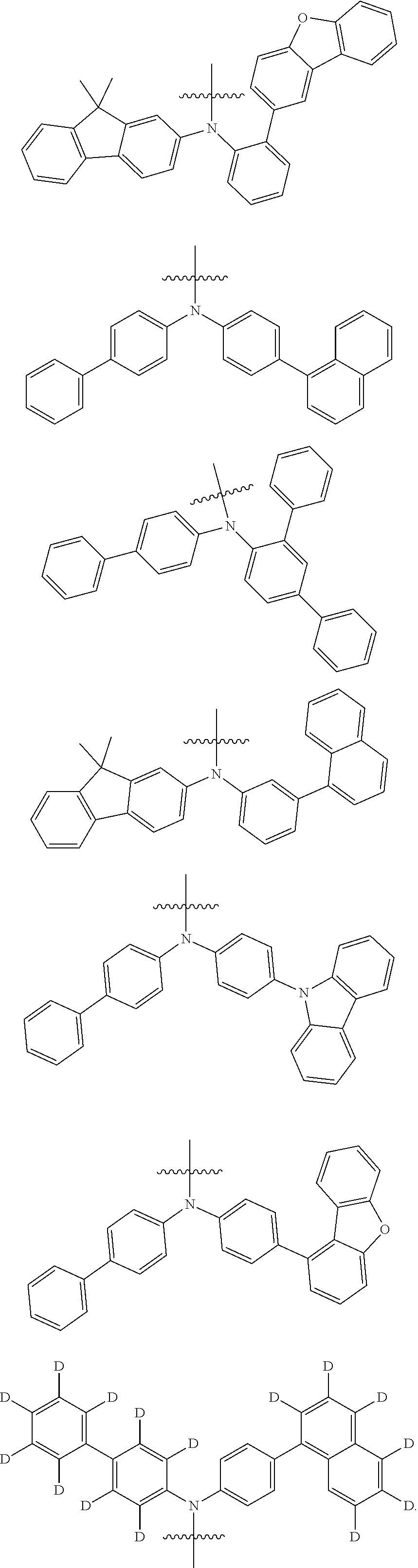

In some embodiments, the aromatic amine compound is selected from the group consisting of the following compounds:

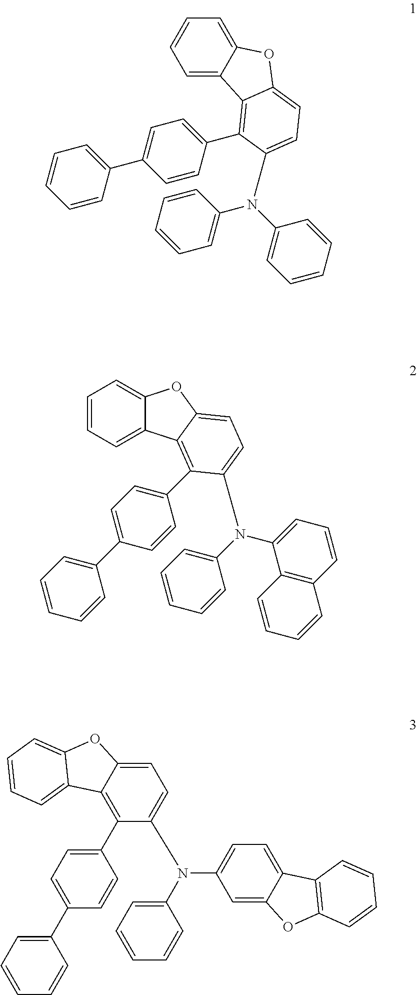 [SMILES: c1ccc(-c2ccc(-c3c(N(c4ccccc4)c4ccccc4)ccc4oc5ccccc5c34)cc2)cc1 ; c1ccc(-c2ccc(-c3c(N(c4ccccc4)c4cccc5ccccc45)ccc4oc5ccccc5c34)cc2)cc1 ; c1ccc(-c2ccc(-c3c(N(c4ccccc4)c4ccc5c(c4)oc4ccccc45)ccc4oc5ccccc5c34)cc2)cc1]

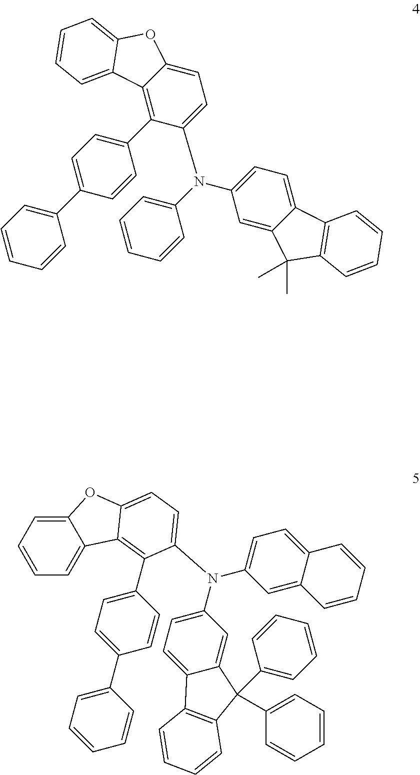 [SMILES: CC1(C)c2ccccc2-c2ccc(N(c3ccccc3)c3ccc4oc5ccccc5c4c3-c3ccc(-c4ccccc4)cc3)cc21 ; c1ccc(-c2ccc(-c3c(N(c4ccc5c(c4)C(c4ccccc4)(c4ccccc4)c4ccccc4-5)c4ccc5ccccc5c4)ccc4oc5ccccc5c34)cc2)cc1]

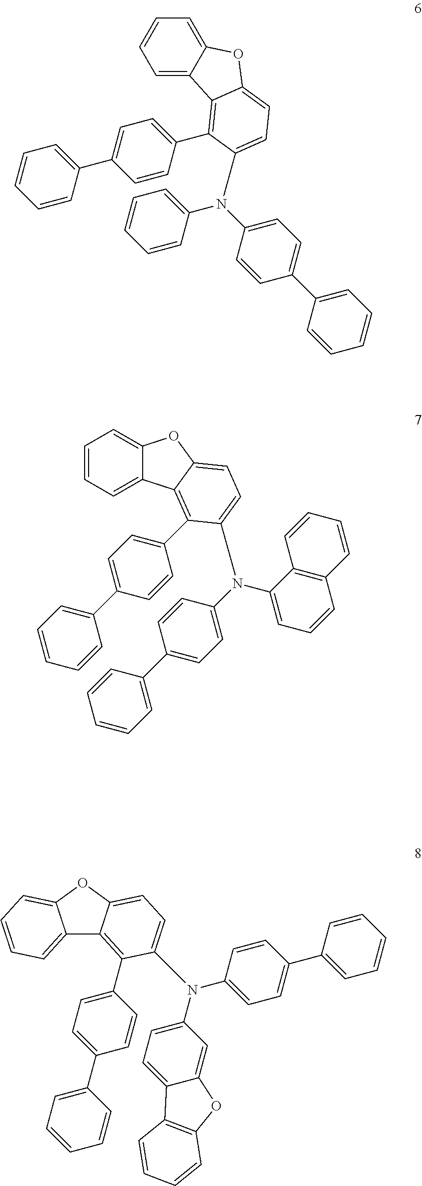 [SMILES: c1ccc(-c2ccc(-c3c(N(c4ccccc4)c4ccc(-c5ccccc5)cc4)ccc4oc5ccccc5c34)cc2)cc1 ; c1ccc(-c2ccc(-c3c(N(c4ccc(-c5ccccc5)cc4)c4cccc5ccccc45)ccc4oc5ccccc5c34)cc2)cc1 ; c1ccc(-c2ccc(-c3c(N(c4ccc(-c5ccccc5)cc4)c4ccc5c(c4)oc4ccccc45)ccc4oc5ccccc5c34)cc2)cc1]

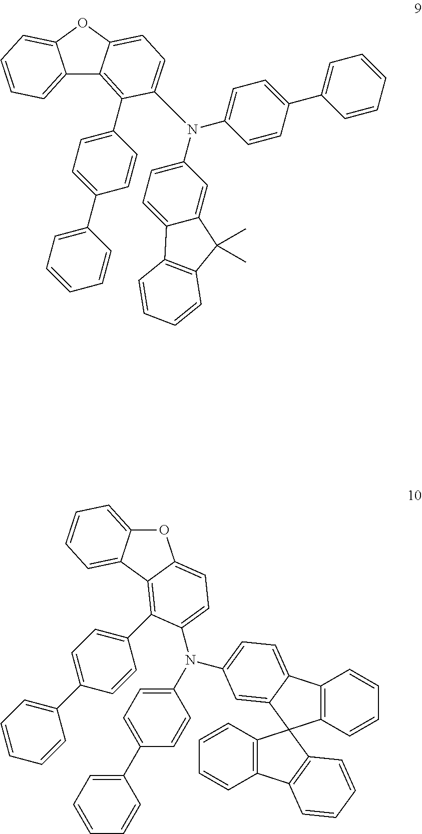 [SMILES: CC1(C)c2ccccc2-c2ccc(N(c3ccc(-c4ccccc4)cc3)c3ccc4oc5ccccc5c4c3-c3ccc(-c4ccccc4)cc3)cc21 ; c1ccc(-c2ccc(-c3c(N(c4ccc(-c5ccccc5)cc4)c4ccc5c(c4)C4(c6ccccc6-c6ccccc64)c4ccccc4-5)ccc4oc5ccccc5c34)cc2)cc1]

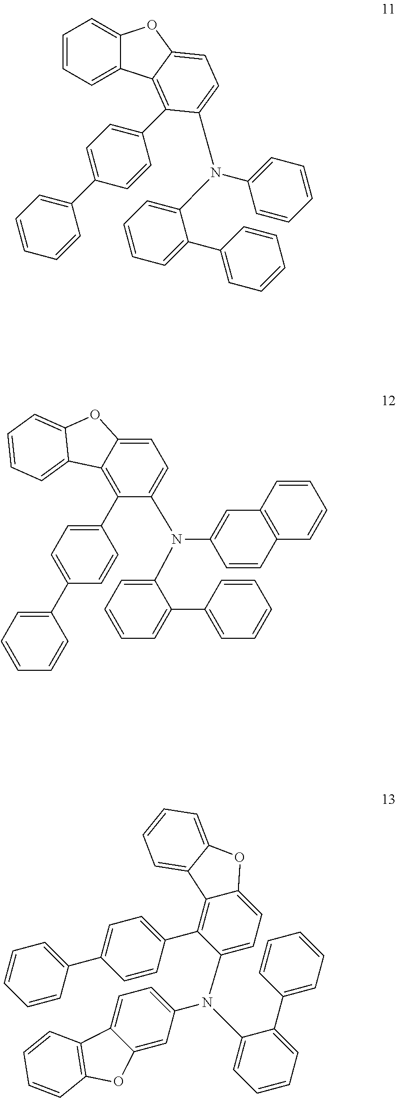 [SMILES: c1ccc(-c2ccc(-c3c(N(c4ccccc4)c4ccccc4-c4ccccc4)ccc4oc5ccccc5c34)cc2)cc1 ; c1ccc(-c2ccc(-c3c(N(c4ccc5ccccc5c4)c4ccccc4-c4ccccc4)ccc4oc5ccccc5c34)cc2)cc1 ; c1ccc(-c2ccc(-c3c(N(c4ccc5c(c4)oc4ccccc45)c4ccccc4-c4ccccc4)ccc4oc5ccccc5c34)cc2)cc1]

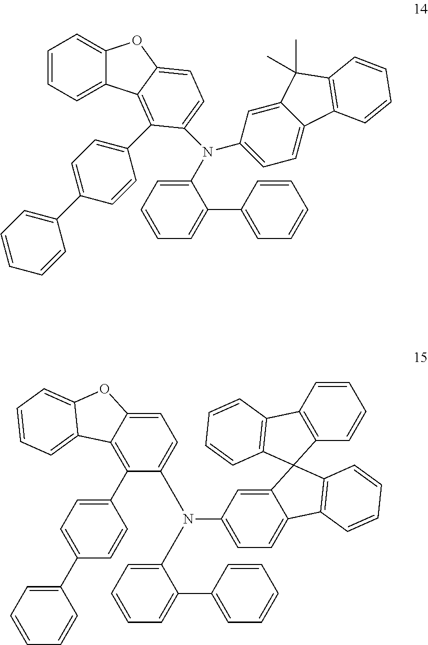 [SMILES: CC1(C)c2ccccc2-c2ccc(N(c3ccccc3-c3ccccc3)c3ccc4oc5ccccc5c4c3-c3ccc(-c4ccccc4)cc3)cc21 ; c1ccc(-c2ccc(-c3c(N(c4ccc5c(c4)C4(c6ccccc6-c6ccccc64)c4ccccc4-5)c4ccccc4-c4ccccc4)ccc4oc5ccccc5c34)cc2)cc1]

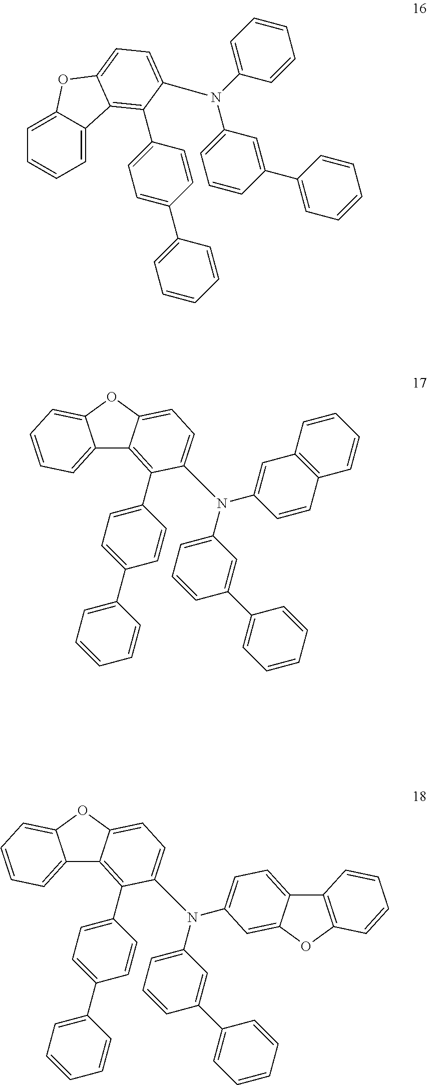 [SMILES: c1ccc(-c2ccc(-c3c(N(c4ccccc4)c4cccc(-c5ccccc5)c4)ccc4oc5ccccc5c34)cc2)cc1 ; c1ccc(-c2ccc(-c3c(N(c4cccc(-c5ccccc5)c4)c4ccc5ccccc5c4)ccc4oc5ccccc5c34)cc2)cc1 ; c1ccc(-c2ccc(-c3c(N(c4cccc(-c5ccccc5)c4)c4ccc5c(c4)oc4ccccc45)ccc4oc5ccccc5c34)cc2)cc1]

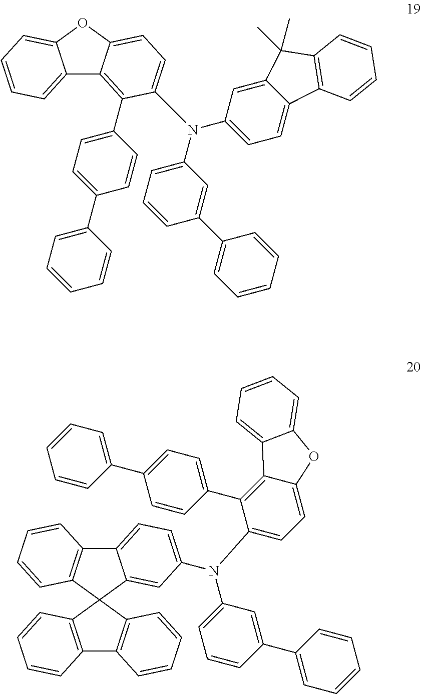 [SMILES: CC1(C)c2ccccc2-c2ccc(N(c3cccc(-c4ccccc4)c3)c3ccc4oc5ccccc5c4c3-c3ccc(-c4ccccc4)cc3)cc21 ; c1ccc(-c2ccc(-c3c(N(c4cccc(-c5ccccc5)c4)c4ccc5c(c4)C4(c6ccccc6-c6ccccc64)c4ccccc4-5)ccc4oc5ccccc5c34)cc2)cc1]

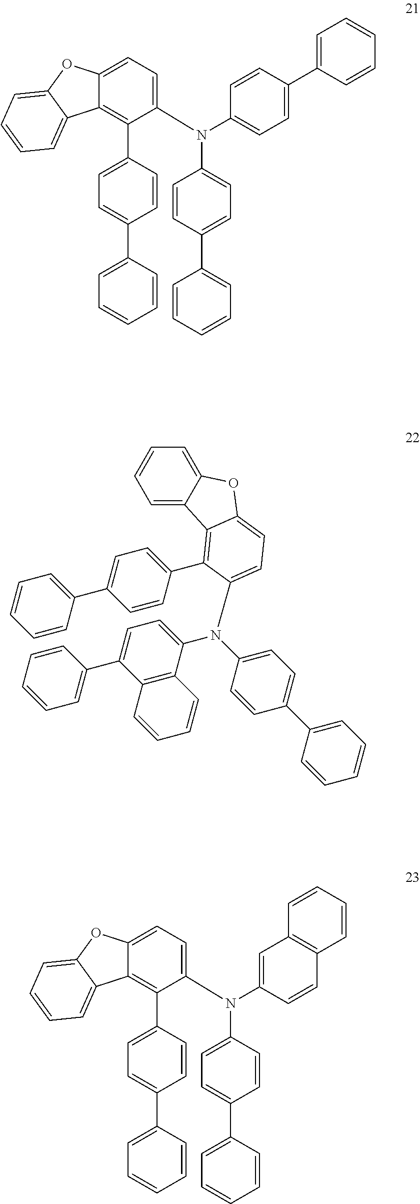 [SMILES: c1ccc(-c2ccc(-c3c(N(c4ccc(-c5ccccc5)cc4)c4ccc(-c5ccccc5)cc4)ccc4oc5ccccc5c34)cc2)cc1 ; c1ccc(-c2ccc(-c3c(N(c4ccc(-c5ccccc5)cc4)c4ccc(-c5ccccc5)c5ccccc45)ccc4oc5ccccc5c34)cc2)cc1 ; c1ccc(-c2ccc(-c3c(N(c4ccc(-c5ccccc5)cc4)c4ccc5ccccc5c4)ccc4oc5ccccc5c34)cc2)cc1]

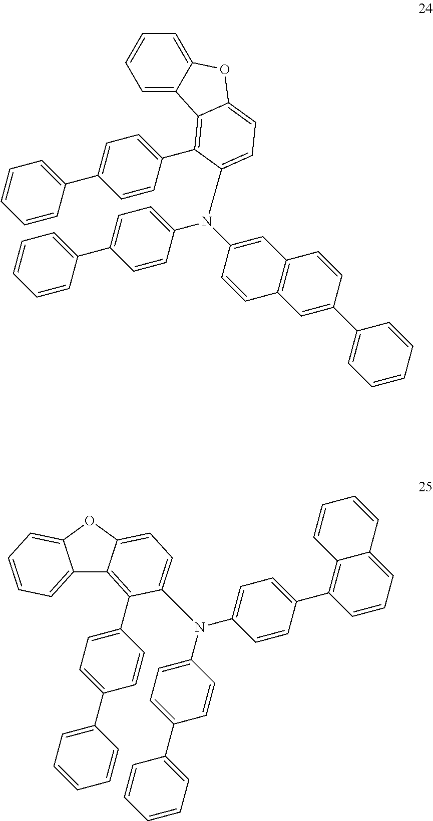 [SMILES: c1ccc(-c2ccc(-c3c(N(c4ccc(-c5ccccc5)cc4)c4ccc5cc(-c6ccccc6)ccc5c4)ccc4oc5ccccc5c34)cc2)cc1 ; c1ccc(-c2ccc(-c3c(N(c4ccc(-c5ccccc5)cc4)c4ccc(-c5cccc6ccccc56)cc4)ccc4oc5ccccc5c34)cc2)cc1]

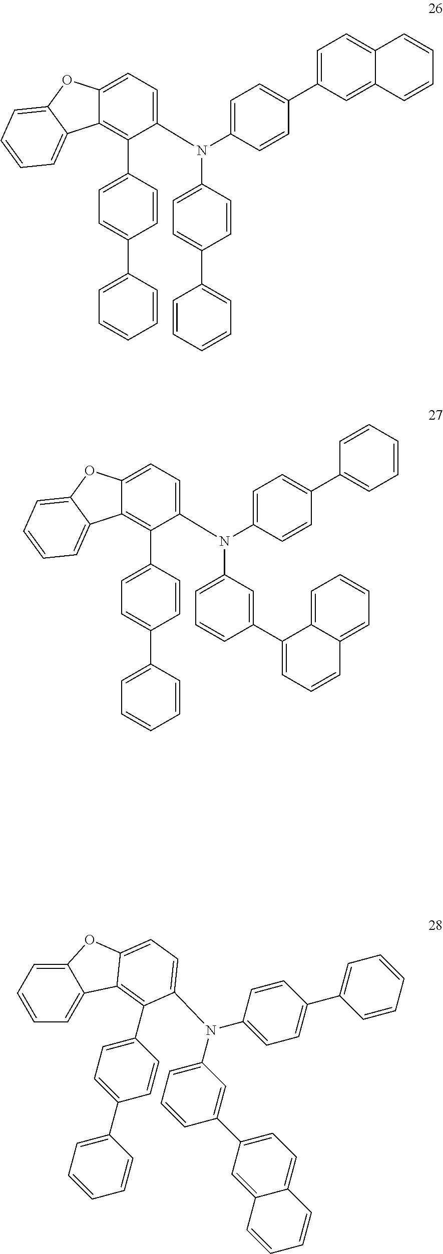 [SMILES: c1ccc(-c2ccc(-c3c(N(c4ccc(-c5ccccc5)cc4)c4ccc(-c5ccc6ccccc6c5)cc4)ccc4oc5ccccc5c34)cc2)cc1 ; c1ccc(-c2ccc(-c3c(N(c4ccc(-c5ccccc5)cc4)c4cccc(-c5cccc6ccccc56)c4)ccc4oc5ccccc5c34)cc2)cc1 ; c1ccc(-c2ccc(-c3c(N(c4ccc(-c5ccccc5)cc4)c4cccc(-c5ccc6ccccc6c5)c4)ccc4oc5ccccc5c34)cc2)cc1]

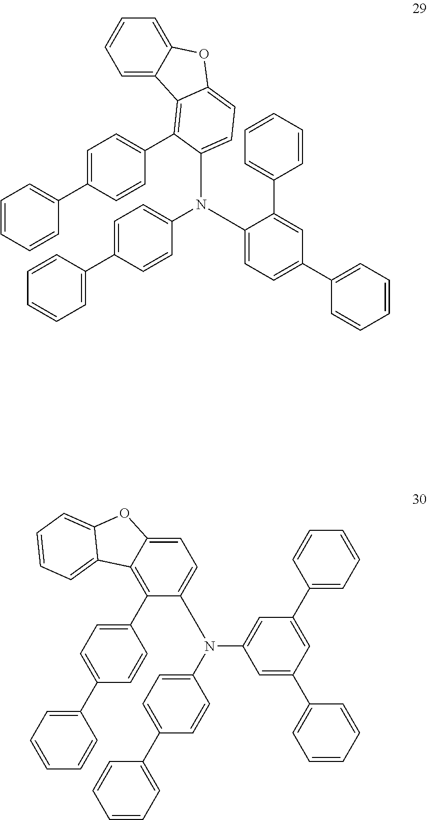 [SMILES: c1ccc(-c2ccc(-c3c(N(c4ccc(-c5ccccc5)cc4)c4ccc(-c5ccccc5)cc4-c4ccccc4)ccc4oc5ccccc5c34)cc2)cc1 ; c1ccc(-c2ccc(-c3c(N(c4ccc(-c5ccccc5)cc4)c4cc(-c5ccccc5)cc(-c5ccccc5)c4)ccc4oc5ccccc5c34)cc2)cc1]

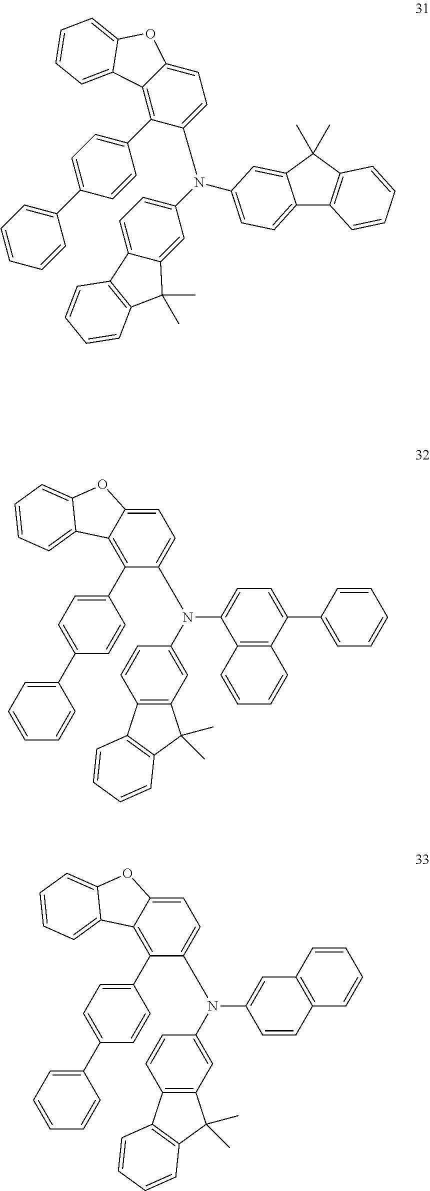 [SMILES: CC1(C)c2ccccc2-c2ccc(N(c3ccc4c(c3)C(C)(C)c3ccccc3-4)c3ccc4oc5ccccc5c4c3-c3ccc(-c4ccccc4)cc3)cc21 ; CC1(C)c2ccccc2-c2ccc(N(c3ccc4oc5ccccc5c4c3-c3ccc(-c4ccccc4)cc3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc(-c4ccccc4)cc3)cc21]

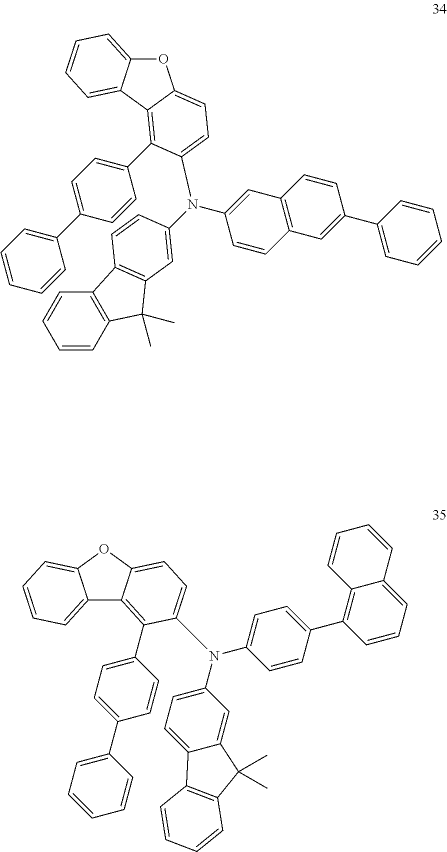 [SMILES: CC1(C)c2ccccc2-c2ccc(N(c3ccc4cc(-c5ccccc5)ccc4c3)c3ccc4oc5ccccc5c4c3-c3ccc(-c4ccccc4)cc3)cc21 ; CC1(C)c2ccccc2-c2ccc(N(c3ccc(-c4cccc5ccccc45)cc3)c3ccc4oc5ccccc5c4c3-c3ccc(-c4ccccc4)cc3)cc21]

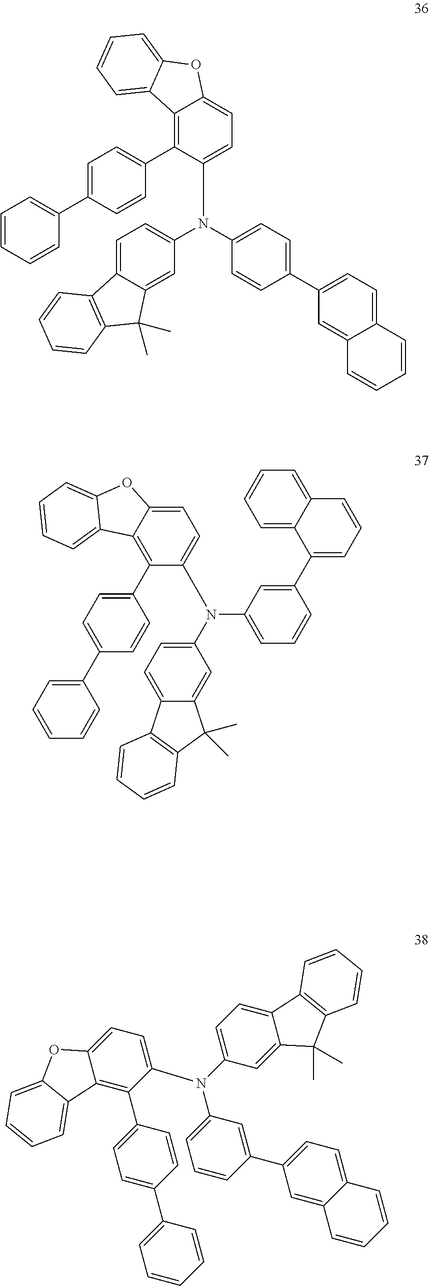 [SMILES: CC1(C)c2ccccc2-c2ccc(N(c3ccc(-c4ccc5ccccc5c4)cc3)c3ccc4oc5ccccc5c4c3-c3ccc(-c4ccccc4)cc3)cc21 ; CC1(C)c2ccccc2-c2ccc(N(c3cccc(-c4cccc5ccccc45)c3)c3ccc4oc5ccccc5c4c3-c3ccc(-c4ccccc4)cc3)cc21 ; CC1(C)c2ccccc2-c2ccc(N(c3cccc(-c4ccc5ccccc5c4)c3)c3ccc4oc5ccccc5c4c3-c3ccc(-c4ccccc4)cc3)cc21]

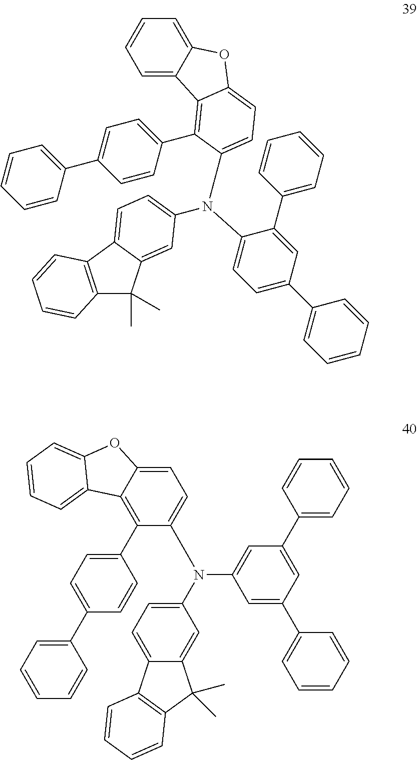 [SMILES: CC1(C)c2ccccc2-c2ccc(N(c3ccc(-c4ccccc4)cc3-c3ccccc3)c3ccc4oc5ccccc5c4c3-c3ccc(-c4ccccc4)cc3)cc21 ; CC1(C)c2ccccc2-c2ccc(N(c3cc(-c4ccccc4)cc(-c4ccccc4)c3)c3ccc4oc5ccccc5c4c3-c3ccc(-c4ccccc4)cc3)cc21]

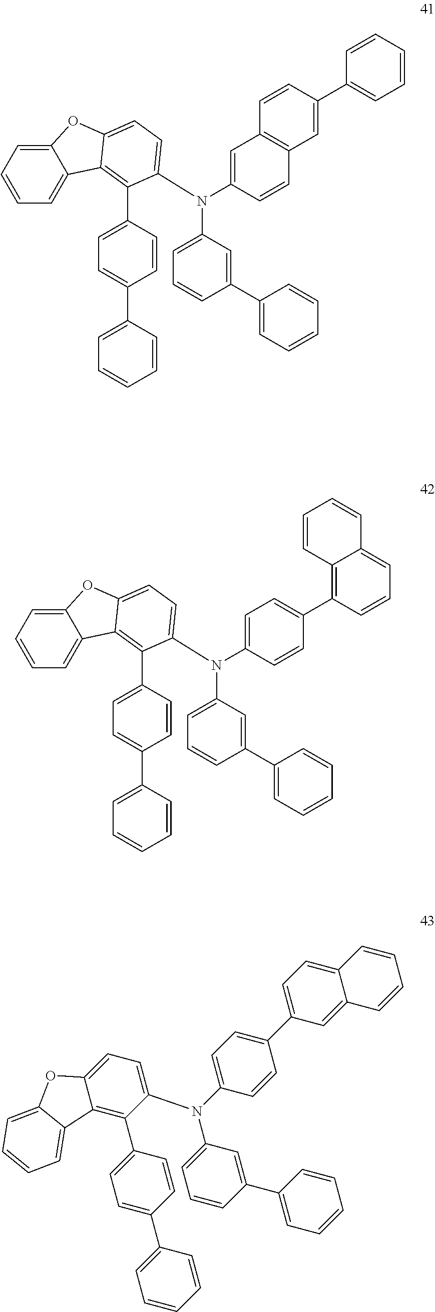 [SMILES: c1ccc(-c2ccc(-c3c(N(c4cccc(-c5ccccc5)c4)c4ccc5cc(-c6ccccc6)ccc5c4)ccc4oc5ccccc5c34)cc2)cc1 ; c1ccc(-c2ccc(-c3c(N(c4ccc(-c5cccc6ccccc56)cc4)c4cccc(-c5ccccc5)c4)ccc4oc5ccccc5c34)cc2)cc1 ; c1ccc(-c2ccc(-c3c(N(c4ccc(-c5ccc6ccccc6c5)cc4)c4cccc(-c5ccccc5)c4)ccc4oc5ccccc5c34)cc2)cc1]

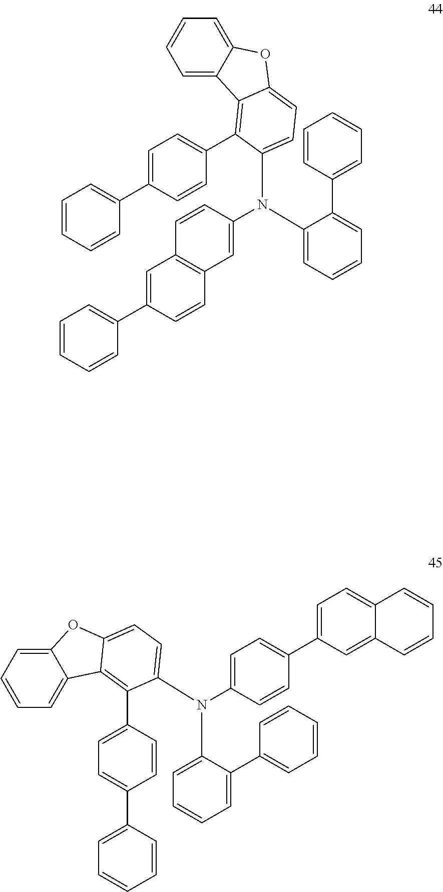 [SMILES: c1ccc(-c2ccc(-c3c(N(c4ccc5cc(-c6ccccc6)ccc5c4)c4ccccc4-c4ccccc4)ccc4oc5ccccc5c34)cc2)cc1 ; c1ccc(-c2ccc(-c3c(N(c4ccc(-c5ccc6ccccc6c5)cc4)c4ccccc4-c4ccccc4)ccc4oc5ccccc5c34)cc2)cc1]

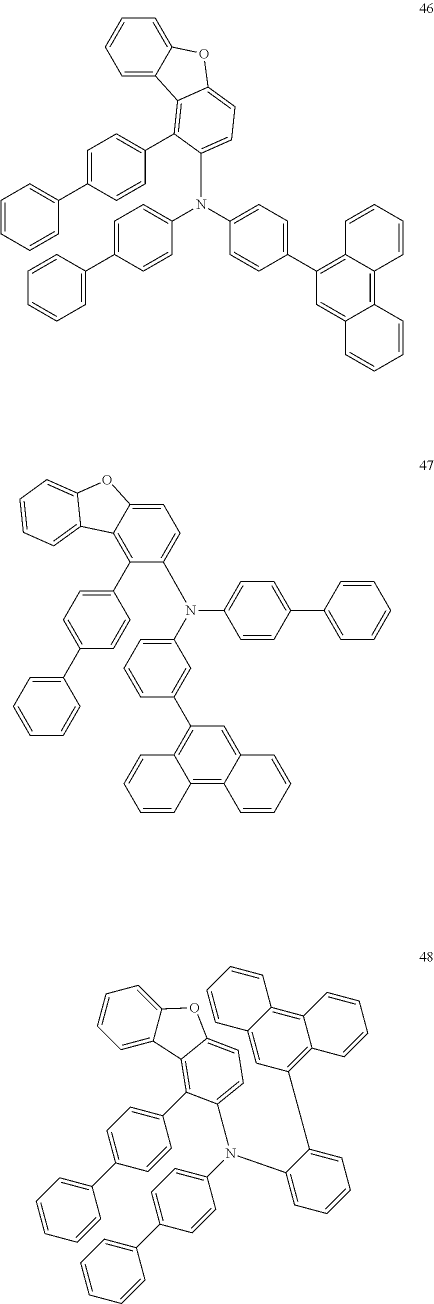 [SMILES: c1ccc(-c2ccc(-c3c(N(c4ccc(-c5ccccc5)cc4)c4ccc(-c5cc6ccccc6c6ccccc56)cc4)ccc4oc5ccccc5c34)cc2)cc1 ; c1ccc(-c2ccc(-c3c(N(c4ccc(-c5ccccc5)cc4)c4cccc(-c5cc6ccccc6c6ccccc56)c4)ccc4oc5ccccc5c34)cc2)cc1 ; c1ccc(-c2ccc(-c3c(N(c4ccc(-c5ccccc5)cc4)c4ccccc4-c4cc5ccccc5c5ccccc45)ccc4oc5ccccc5c34)cc2)cc1]

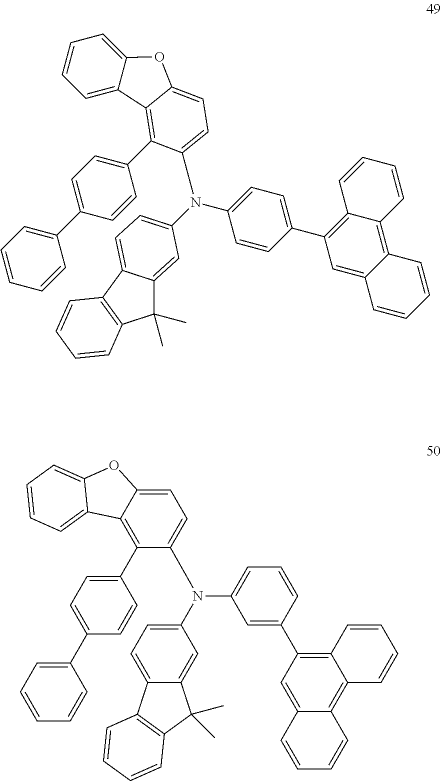 [SMILES: CC1(C)c2ccccc2-c2ccc(N(c3ccc(-c4cc5ccccc5c5ccccc45)cc3)c3ccc4oc5ccccc5c4c3-c3ccc(-c4ccccc4)cc3)cc21 ; CC1(C)c2ccccc2-c2ccc(N(c3cccc(-c4cc5ccccc5c5ccccc45)c3)c3ccc4oc5ccccc5c4c3-c3ccc(-c4ccccc4)cc3)cc21]

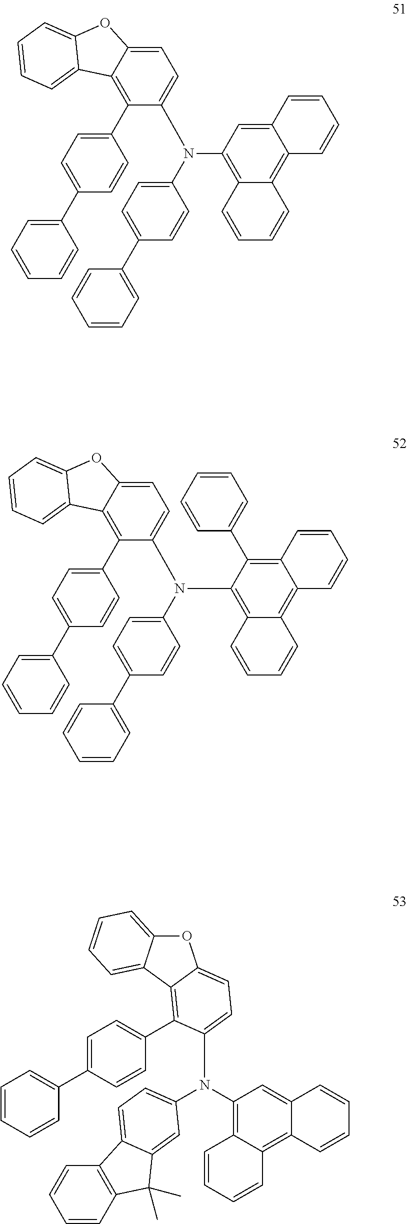 [SMILES: c1ccc(-c2ccc(-c3c(N(c4ccc(-c5ccccc5)cc4)c4cc5ccccc5c5ccccc45)ccc4oc5ccccc5c34)cc2)cc1 ; c1ccc(-c2ccc(-c3c(N(c4ccc(-c5ccccc5)cc4)c4c(-c5ccccc5)c5ccccc5c5ccccc45)ccc4oc5ccccc5c34)cc2)cc1 ; CC1(C)c2ccccc2-c2ccc(N(c3ccc4oc5ccccc5c4c3-c3ccc(-c4ccccc4)cc3)c3cc4ccccc4c4ccccc34)cc21]

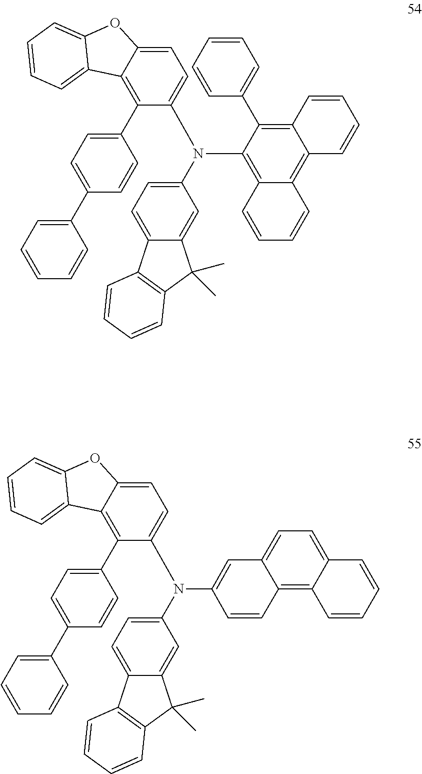 [SMILES: CC1(C)c2ccccc2-c2ccc(N(c3ccc4oc5ccccc5c4c3-c3ccc(-c4ccccc4)cc3)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3ccc(-c4ccccc4)cc3)cc21]

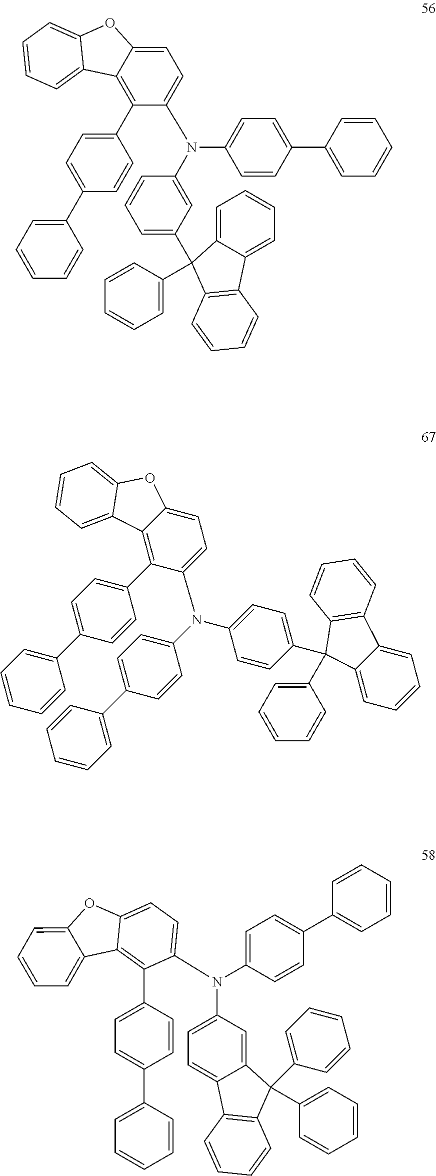 [SMILES: c1ccc(-c2ccc(-c3c(N(c4ccc(-c5ccccc5)cc4)c4cccc(C5(c6ccccc6)c6ccccc6-c6ccccc65)c4)ccc4oc5ccccc5c34)cc2)cc1 ; c1ccc(-c2ccc(-c3c(N(c4ccc(-c5ccccc5)cc4)c4ccc(C5(c6ccccc6)c6ccccc6-c6ccccc65)cc4)ccc4oc5ccccc5c34)cc2)cc1 ; c1ccc(-c2ccc(-c3c(N(c4ccc(-c5ccccc5)cc4)c4ccc5c(c4)C(c4ccccc4)(c4ccccc4)c4ccccc4-5)ccc4oc5ccccc5c34)cc2)cc1]

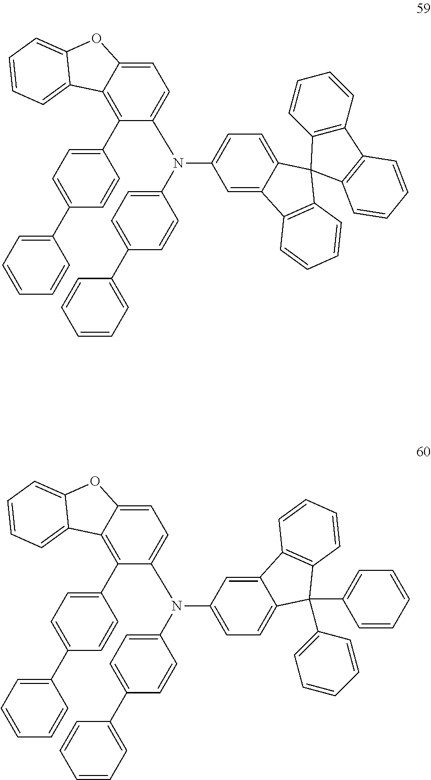 [SMILES: c1ccc(-c2ccc(-c3c(N(c4ccc(-c5ccccc5)cc4)c4ccc5c(c4)-c4ccccc4C54c5ccccc5-c5ccccc54)ccc4oc5ccccc5c34)cc2)cc1 ; c1ccc(-c2ccc(-c3c(N(c4ccc(-c5ccccc5)cc4)c4ccc5c(c4)-c4ccccc4C5(c4ccccc4)c4ccccc4)ccc4oc5ccccc5c34)cc2)cc1]

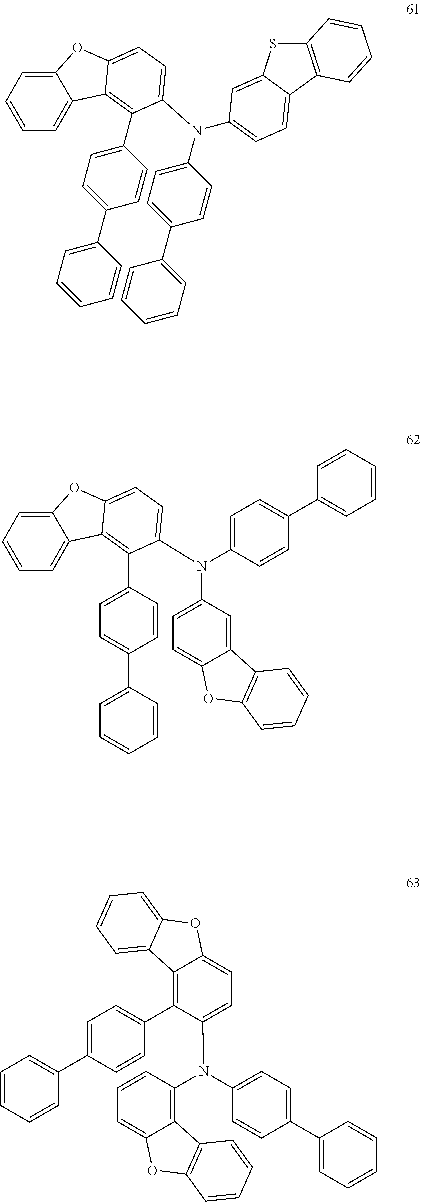 [SMILES: c1ccc(-c2ccc(-c3c(N(c4ccc(-c5ccccc5)cc4)c4ccc5c(c4)sc4ccccc45)ccc4oc5ccccc5c34)cc2)cc1 ; c1ccc(-c2ccc(-c3c(N(c4ccc(-c5ccccc5)cc4)c4ccc5oc6ccccc6c5c4)ccc4oc5ccccc5c34)cc2)cc1 ; c1ccc(-c2ccc(-c3c(N(c4ccc(-c5ccccc5)cc4)c4cccc5oc6ccccc6c45)ccc4oc5ccccc5c34)cc2)cc1]

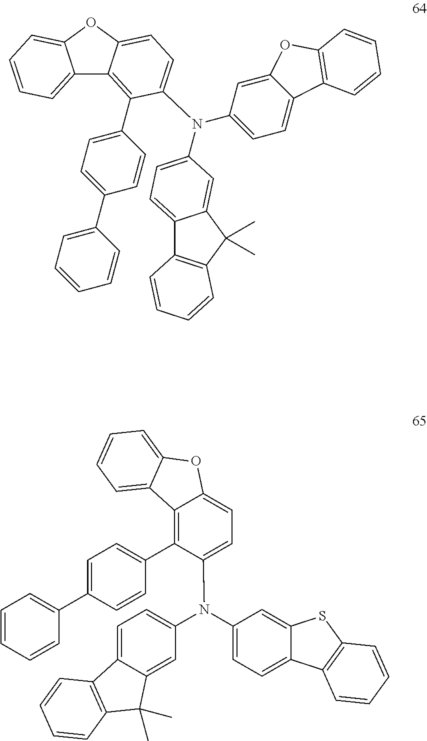 [SMILES: CC1(C)c2ccccc2-c2ccc(N(c3ccc4c(c3)oc3ccccc34)c3ccc4oc5ccccc5c4c3-c3ccc(-c4ccccc4)cc3)cc21 ; CC1(C)c2ccccc2-c2ccc(N(c3ccc4c(c3)sc3ccccc34)c3ccc4oc5ccccc5c4c3-c3ccc(-c4ccccc4)cc3)cc21]

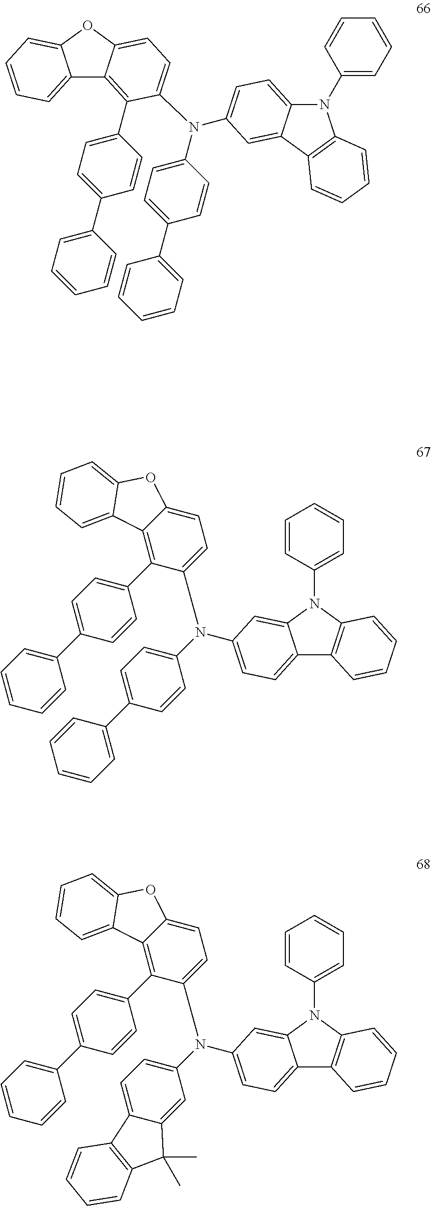 [SMILES: c1ccc(-c2ccc(-c3c(N(c4ccc(-c5ccccc5)cc4)c4ccc5c(c4)c4ccccc4n5-c4ccccc4)ccc4oc5ccccc5c34)cc2)cc1 ; c1ccc(-c2ccc(-c3c(N(c4ccc(-c5ccccc5)cc4)c4ccc5c6ccccc6n(-c6ccccc6)c5c4)ccc4oc5ccccc5c34)cc2)cc1 ; CC1(C)c2ccccc2-c2ccc(N(c3ccc4c5ccccc5n(-c5ccccc5)c4c3)c3ccc4oc5ccccc5c4c3-c3ccc(-c4ccccc4)cc3)cc21]

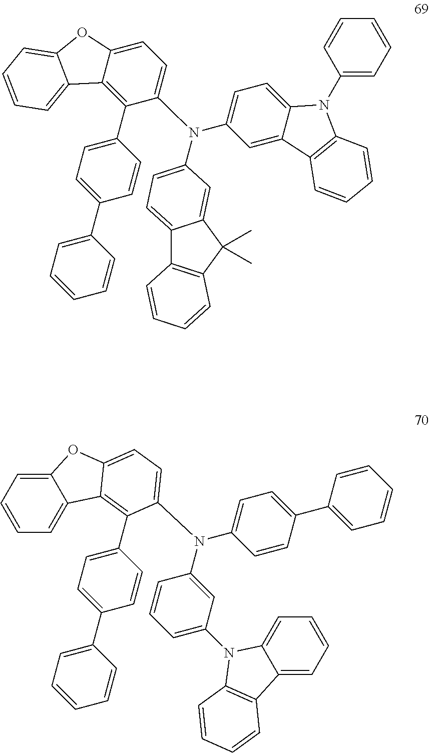 [SMILES: CC1(C)c2ccccc2-c2ccc(N(c3ccc4c(c3)c3ccccc3n4-c3ccccc3)c3ccc4oc5ccccc5c4c3-c3ccc(-c4ccccc4)cc3)cc21 ; c1ccc(-c2ccc(-c3c(N(c4ccc(-c5ccccc5)cc4)c4cccc(-n5c6ccccc6c6ccccc65)c4)ccc4oc5ccccc5c34)cc2)cc1]

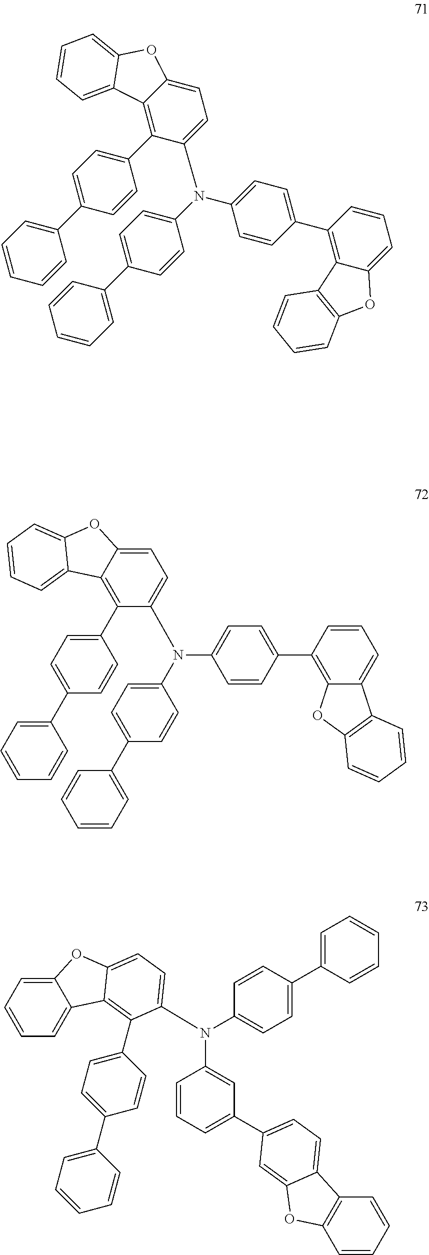 [SMILES: c1ccc(-c2ccc(-c3c(N(c4ccc(-c5ccccc5)cc4)c4ccc(-c5cccc6oc7ccccc7c56)cc4)ccc4oc5ccccc5c34)cc2)cc1 ; c1ccc(-c2ccc(-c3c(N(c4ccc(-c5ccccc5)cc4)c4ccc(-c5cccc6c5oc5ccccc56)cc4)ccc4oc5ccccc5c34)cc2)cc1 ; c1ccc(-c2ccc(-c3c(N(c4ccc(-c5ccccc5)cc4)c4cccc(-c5ccc6c(c5)oc5ccccc56)c4)ccc4oc5ccccc5c34)cc2)cc1]

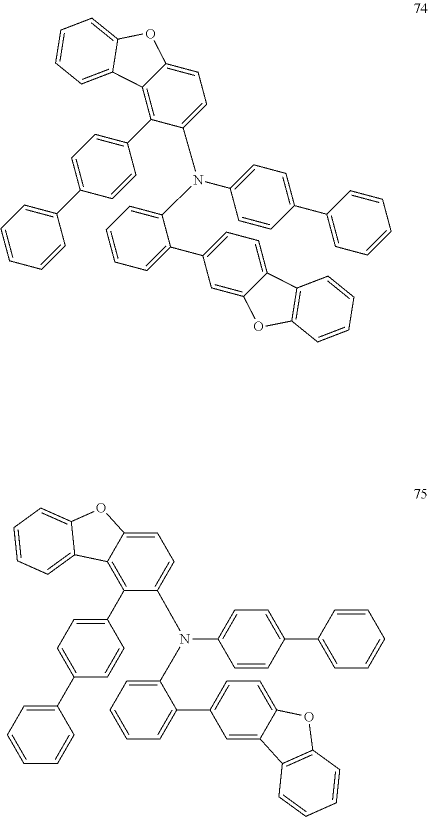 [SMILES: c1ccc(-c2ccc(-c3c(N(c4ccc(-c5ccccc5)cc4)c4ccccc4-c4ccc5c(c4)oc4ccccc45)ccc4oc5ccccc5c34)cc2)cc1 ; c1ccc(-c2ccc(-c3c(N(c4ccc(-c5ccccc5)cc4)c4ccccc4-c4ccc5oc6ccccc6c5c4)ccc4oc5ccccc5c34)cc2)cc1]

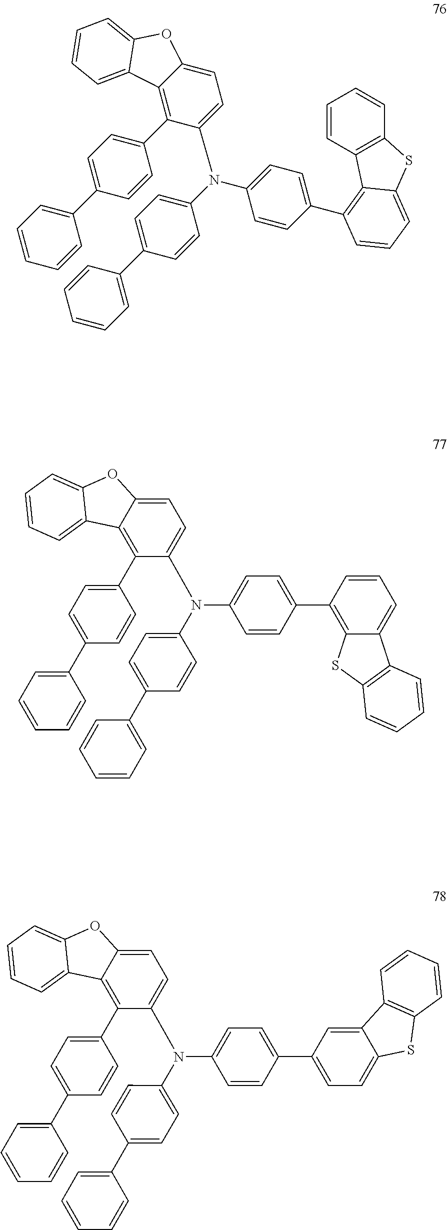 [SMILES: c1ccc(-c2ccc(-c3c(N(c4ccc(-c5ccccc5)cc4)c4ccc(-c5cccc6sc7ccccc7c56)cc4)ccc4oc5ccccc5c34)cc2)cc1 ; c1ccc(-c2ccc(-c3c(N(c4ccc(-c5ccccc5)cc4)c4ccc(-c5cccc6c5sc5ccccc56)cc4)ccc4oc5ccccc5c34)cc2)cc1 ; c1ccc(-c2ccc(-c3c(N(c4ccc(-c5ccccc5)cc4)c4ccc(-c5ccc6sc7ccccc7c6c5)cc4)ccc4oc5ccccc5c34)cc2)cc1]

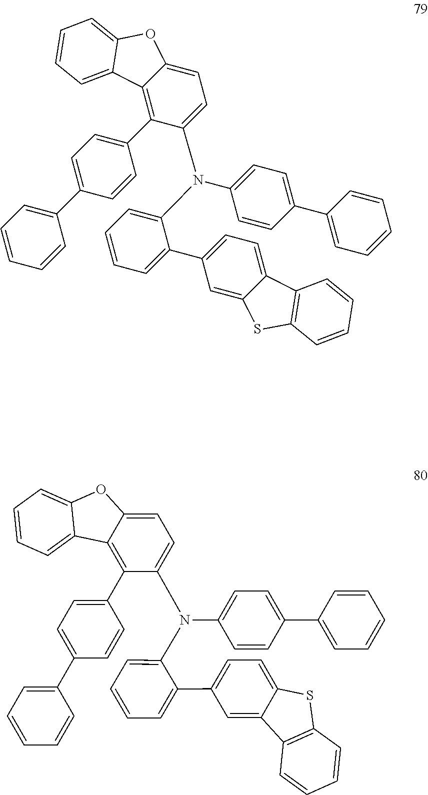 [SMILES: c1ccc(-c2ccc(-c3c(N(c4ccc(-c5ccccc5)cc4)c4ccccc4-c4ccc5c(c4)sc4ccccc45)ccc4oc5ccccc5c34)cc2)cc1 ; c1ccc(-c2ccc(-c3c(N(c4ccc(-c5ccccc5)cc4)c4ccccc4-c4ccc5sc6ccccc6c5c4)ccc4oc5ccccc5c34)cc2)cc1]

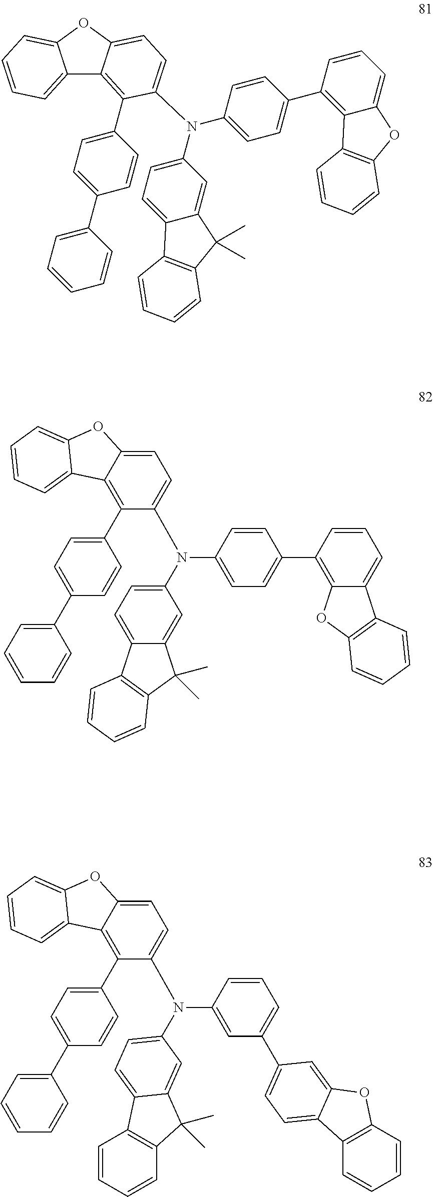 [SMILES: CC1(C)c2ccccc2-c2ccc(N(c3ccc(-c4cccc5oc6ccccc6c45)cc3)c3ccc4oc5ccccc5c4c3-c3ccc(-c4ccccc4)cc3)cc21 ; CC1(C)c2ccccc2-c2ccc(N(c3ccc(-c4cccc5c4oc4ccccc45)cc3)c3ccc4oc5ccccc5c4c3-c3ccc(-c4ccccc4)cc3)cc21 ; CC1(C)c2ccccc2-c2ccc(N(c3cccc(-c4ccc5c(c4)oc4ccccc45)c3)c3ccc4oc5ccccc5c4c3-c3ccc(-c4ccccc4)cc3)cc21]

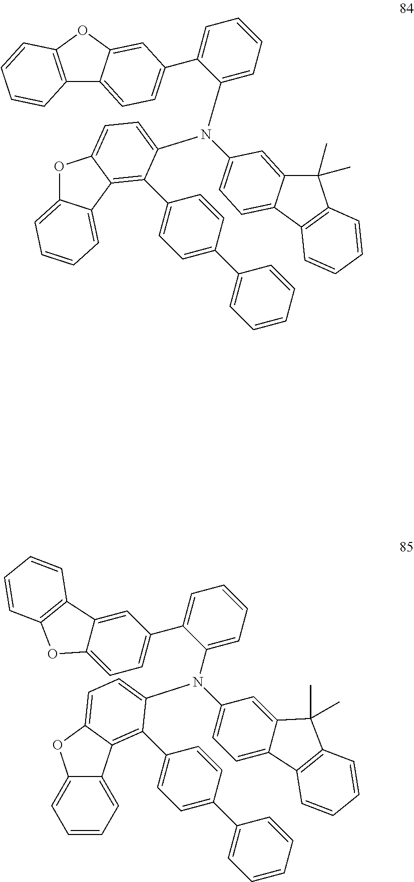 [SMILES: CC1(C)c2ccccc2-c2ccc(N(c3ccccc3-c3ccc4c(c3)oc3ccccc34)c3ccc4oc5ccccc5c4c3-c3ccc(-c4ccccc4)cc3)cc21 ; CC1(C)c2ccccc2-c2ccc(N(c3ccccc3-c3ccc4oc5ccccc5c4c3)c3ccc4oc5ccccc5c4c3-c3ccc(-c4ccccc4)cc3)cc21]

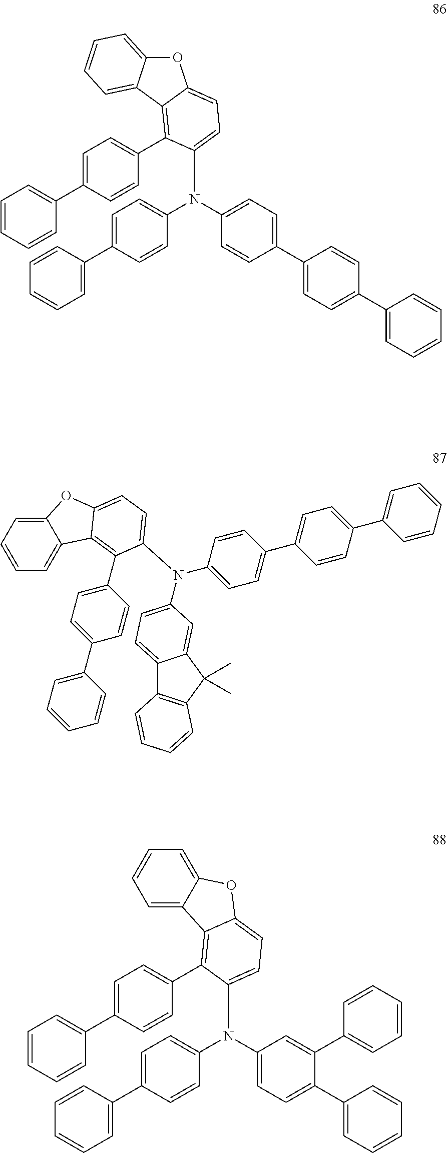 [SMILES: c1ccc(-c2ccc(-c3ccc(N(c4ccc(-c5ccccc5)cc4)c4ccc5oc6ccccc6c5c4-c4ccc(-c5ccccc5)cc4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc(-c4ccccc4)cc3)cc21 ; c1ccc(-c2ccc(-c3c(N(c4ccc(-c5ccccc5)cc4)c4ccc(-c5ccccc5)c(-c5ccccc5)c4)ccc4oc5ccccc5c34)cc2)cc1]

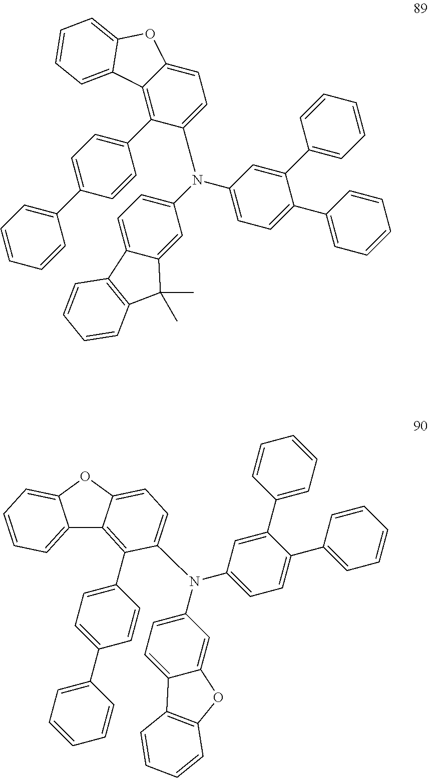 [SMILES: CC1(C)c2ccccc2-c2ccc(N(c3ccc(-c4ccccc4)c(-c4ccccc4)c3)c3ccc4oc5ccccc5c4c3-c3ccc(-c4ccccc4)cc3)cc21 ; c1ccc(-c2ccc(-c3c(N(c4ccc(-c5ccccc5)c(-c5ccccc5)c4)c4ccc5c(c4)oc4ccccc45)ccc4oc5ccccc5c34)cc2)cc1]

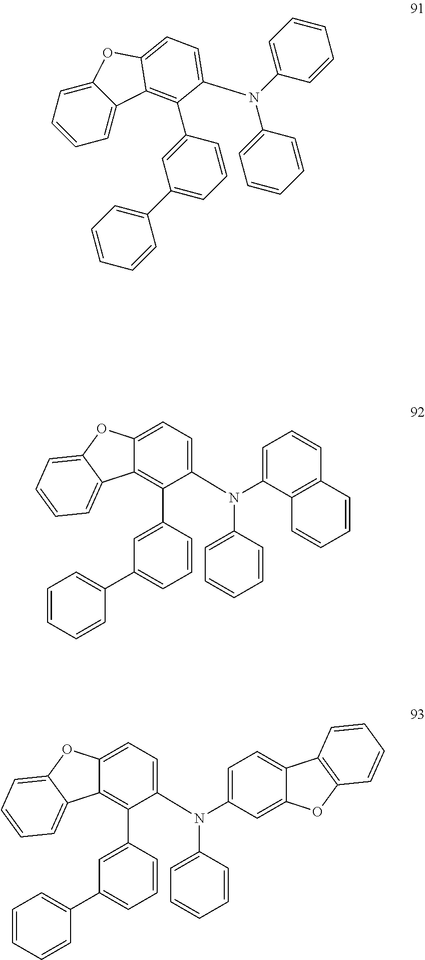 [SMILES: c1ccc(-c2cccc(-c3c(N(c4ccccc4)c4ccccc4)ccc4oc5ccccc5c34)c2)cc1 ; c1ccc(-c2cccc(-c3c(N(c4ccccc4)c4cccc5ccccc45)ccc4oc5ccccc5c34)c2)cc1 ; c1ccc(-c2cccc(-c3c(N(c4ccccc4)c4ccc5c(c4)oc4ccccc45)ccc4oc5ccccc5c34)c2)cc1]

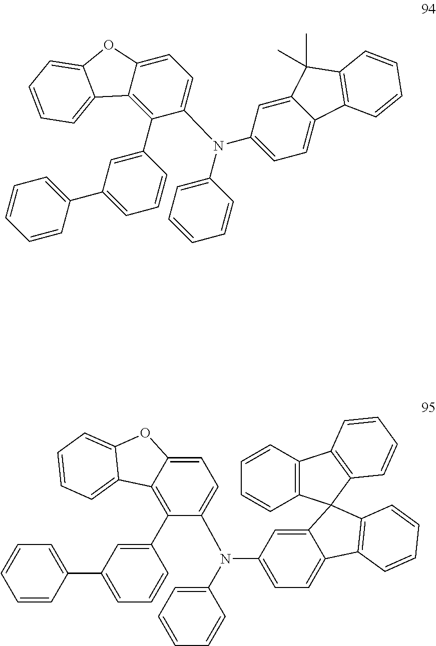 [SMILES: CC1(C)c2ccccc2-c2ccc(N(c3ccccc3)c3ccc4oc5ccccc5c4c3-c3cccc(-c4ccccc4)c3)cc21 ; c1ccc(-c2cccc(-c3c(N(c4ccccc4)c4ccc5c(c4)C4(c6ccccc6-c6ccccc64)c4ccccc4-5)ccc4oc5ccccc5c34)c2)cc1]

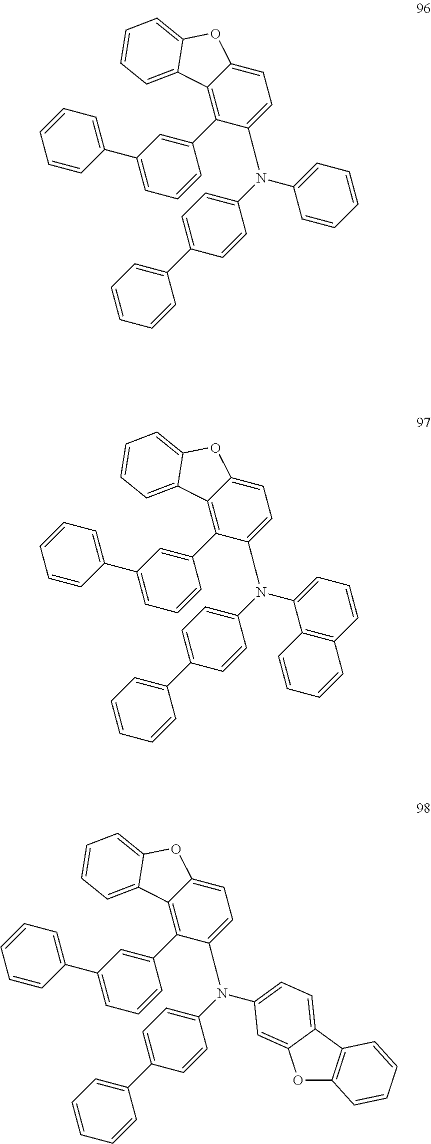 [SMILES: c1ccc(-c2ccc(N(c3ccccc3)c3ccc4oc5ccccc5c4c3-c3cccc(-c4ccccc4)c3)cc2)cc1 ; c1ccc(-c2ccc(N(c3ccc4oc5ccccc5c4c3-c3cccc(-c4ccccc4)c3)c3cccc4ccccc34)cc2)cc1 ; c1ccc(-c2ccc(N(c3ccc4c(c3)oc3ccccc34)c3ccc4oc5ccccc5c4c3-c3cccc(-c4ccccc4)c3)cc2)cc1]

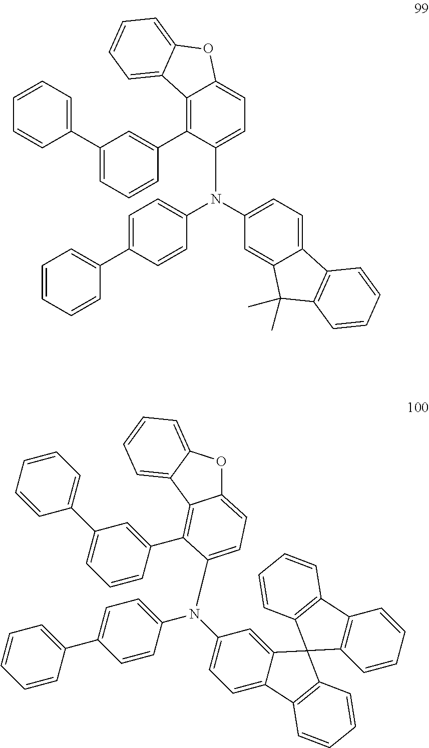 [SMILES: CC1(C)c2ccccc2-c2ccc(N(c3ccc(-c4ccccc4)cc3)c3ccc4oc5ccccc5c4c3-c3cccc(-c4ccccc4)c3)cc21 ; c1ccc(-c2ccc(N(c3ccc4c(c3)C3(c5ccccc5-c5ccccc53)c3ccccc3-4)c3ccc4oc5ccccc5c4c3-c3cccc(-c4ccccc4)c3)cc2)cc1]

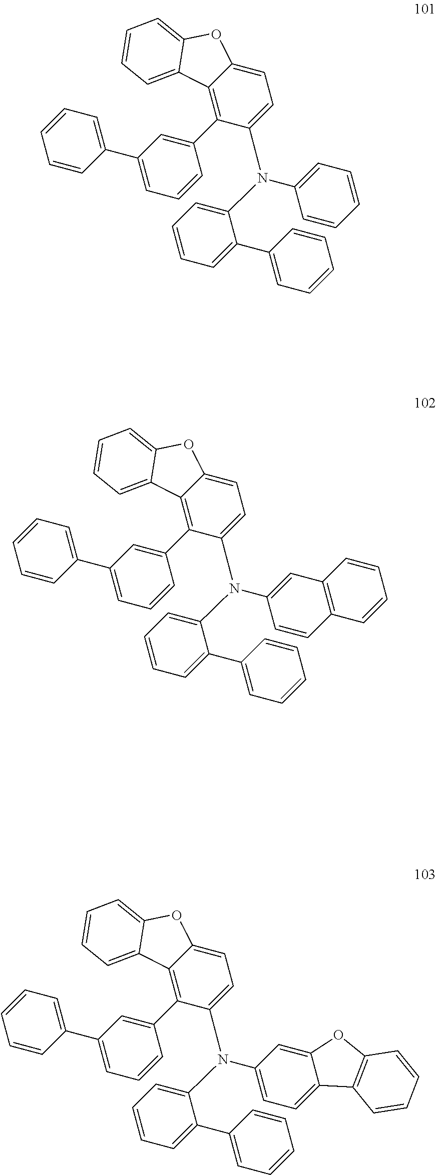 [SMILES: c1ccc(-c2cccc(-c3c(N(c4ccccc4)c4ccccc4-c4ccccc4)ccc4oc5ccccc5c34)c2)cc1 ; c1ccc(-c2cccc(-c3c(N(c4ccc5ccccc5c4)c4ccccc4-c4ccccc4)ccc4oc5ccccc5c34)c2)cc1 ; c1ccc(-c2cccc(-c3c(N(c4ccc5c(c4)oc4ccccc45)c4ccccc4-c4ccccc4)ccc4oc5ccccc5c34)c2)cc1]

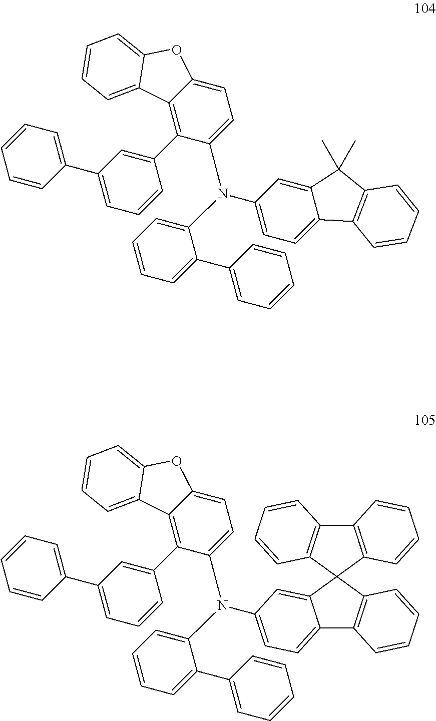 [SMILES: CC1(C)c2ccccc2-c2ccc(N(c3ccccc3-c3ccccc3)c3ccc4oc5ccccc5c4c3-c3cccc(-c4ccccc4)c3)cc21 ; c1ccc(-c2cccc(-c3c(N(c4ccc5c(c4)C4(c6ccccc6-c6ccccc64)c4ccccc4-5)c4ccccc4-c4ccccc4)ccc4oc5ccccc5c34)c2)cc1]

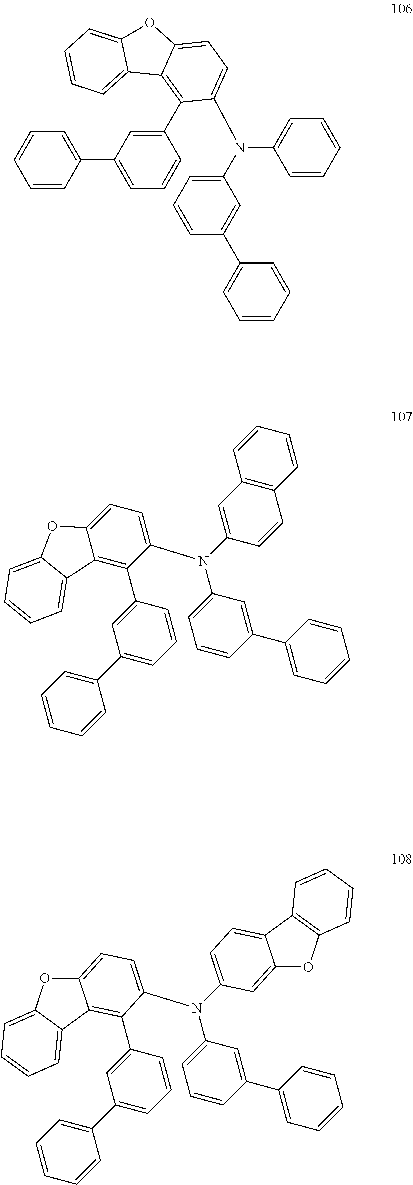 [SMILES: c1ccc(-c2cccc(-c3c(N(c4ccccc4)c4cccc(-c5ccccc5)c4)ccc4oc5ccccc5c34)c2)cc1 ; c1ccc(-c2cccc(-c3c(N(c4cccc(-c5ccccc5)c4)c4ccc5ccccc5c4)ccc4oc5ccccc5c34)c2)cc1 ; c1ccc(-c2cccc(-c3c(N(c4cccc(-c5ccccc5)c4)c4ccc5c(c4)oc4ccccc45)ccc4oc5ccccc5c34)c2)cc1]

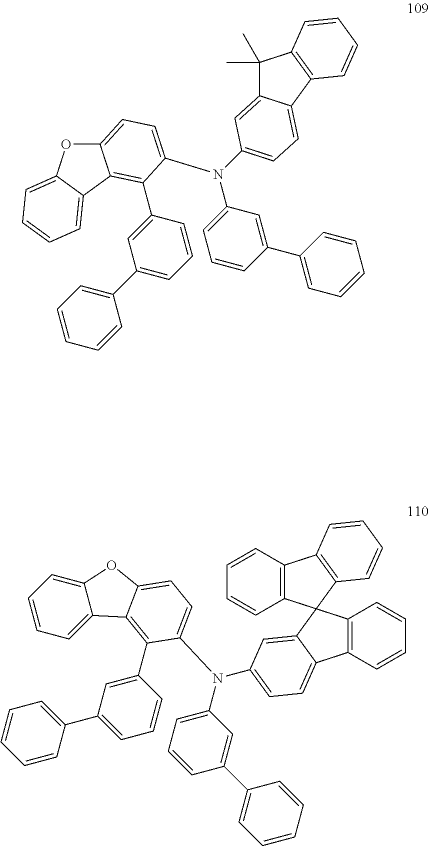 [SMILES: CC1(C)c2ccccc2-c2ccc(N(c3cccc(-c4ccccc4)c3)c3ccc4oc5ccccc5c4c3-c3cccc(-c4ccccc4)c3)cc21 ; c1ccc(-c2cccc(-c3c(N(c4cccc(-c5ccccc5)c4)c4ccc5c(c4)C4(c6ccccc6-c6ccccc64)c4ccccc4-5)ccc4oc5ccccc5c34)c2)cc1]

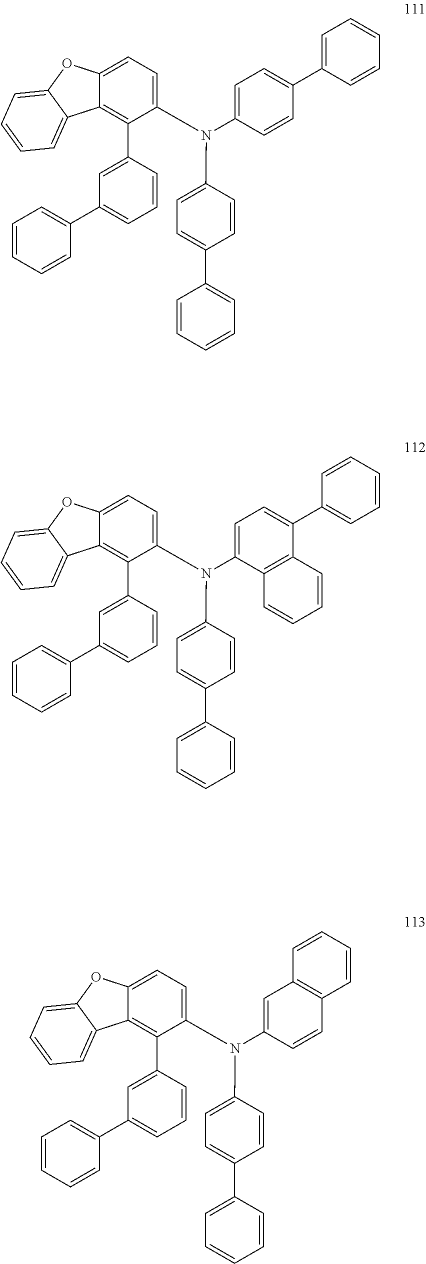 [SMILES: c1ccc(-c2ccc(N(c3ccc(-c4ccccc4)cc3)c3ccc4oc5ccccc5c4c3-c3cccc(-c4ccccc4)c3)cc2)cc1 ; c1ccc(-c2ccc(N(c3ccc4oc5ccccc5c4c3-c3cccc(-c4ccccc4)c3)c3ccc(-c4ccccc4)c4ccccc34)cc2)cc1 ; c1ccc(-c2ccc(N(c3ccc4ccccc4c3)c3ccc4oc5ccccc5c4c3-c3cccc(-c4ccccc4)c3)cc2)cc1]

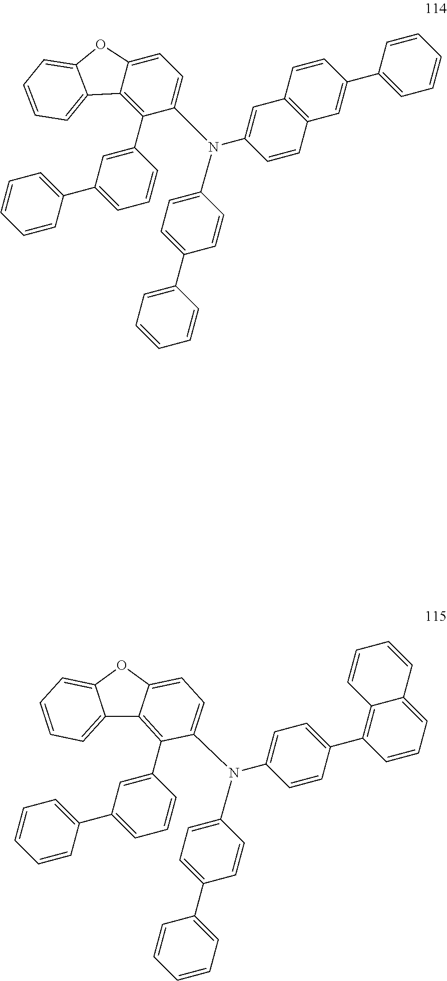 [SMILES: c1ccc(-c2ccc(N(c3ccc4cc(-c5ccccc5)ccc4c3)c3ccc4oc5ccccc5c4c3-c3cccc(-c4ccccc4)c3)cc2)cc1 ; c1ccc(-c2ccc(N(c3ccc(-c4cccc5ccccc45)cc3)c3ccc4oc5ccccc5c4c3-c3cccc(-c4ccccc4)c3)cc2)cc1]

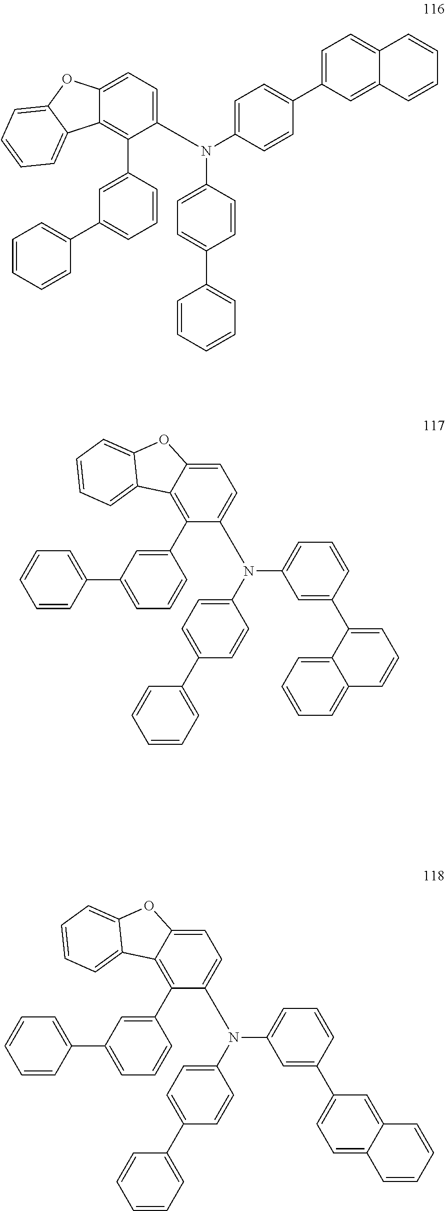 [SMILES: c1ccc(-c2ccc(N(c3ccc(-c4ccc5ccccc5c4)cc3)c3ccc4oc5ccccc5c4c3-c3cccc(-c4ccccc4)c3)cc2)cc1 ; c1ccc(-c2ccc(N(c3cccc(-c4cccc5ccccc45)c3)c3ccc4oc5ccccc5c4c3-c3cccc(-c4ccccc4)c3)cc2)cc1 ; c1ccc(-c2ccc(N(c3cccc(-c4ccc5ccccc5c4)c3)c3ccc4oc5ccccc5c4c3-c3cccc(-c4ccccc4)c3)cc2)cc1]

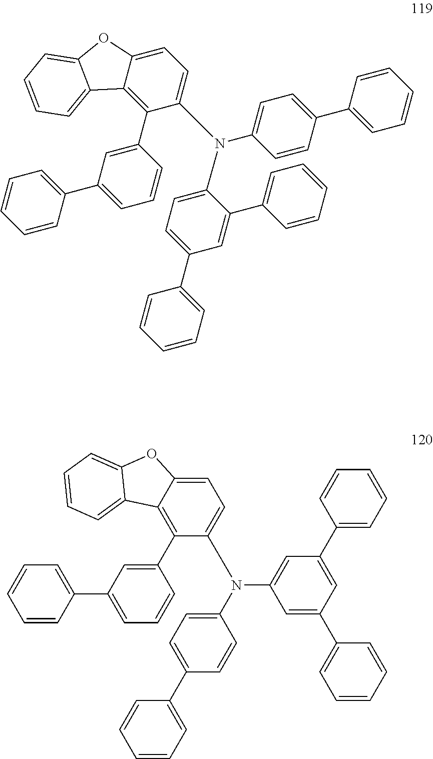 [SMILES: c1ccc(-c2ccc(N(c3ccc(-c4ccccc4)cc3-c3ccccc3)c3ccc4oc5ccccc5c4c3-c3cccc(-c4ccccc4)c3)cc2)cc1 ; c1ccc(-c2ccc(N(c3cc(-c4ccccc4)cc(-c4ccccc4)c3)c3ccc4oc5ccccc5c4c3-c3cccc(-c4ccccc4)c3)cc2)cc1]

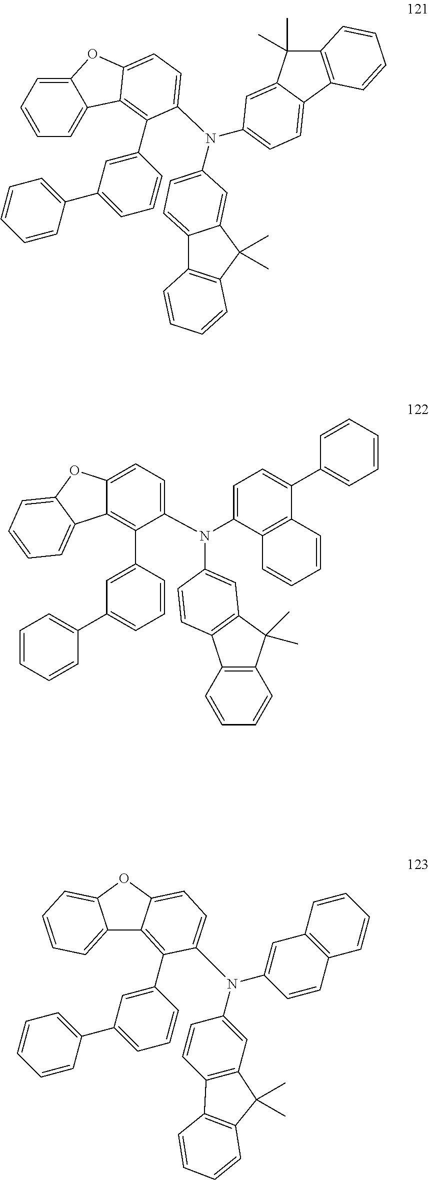 [SMILES: CC1(C)c2ccccc2-c2ccc(N(c3ccc4c(c3)C(C)(C)c3ccccc3-4)c3ccc4oc5ccccc5c4c3-c3cccc(-c4ccccc4)c3)cc21 ; CC1(C)c2ccccc2-c2ccc(N(c3ccc4oc5ccccc5c4c3-c3cccc(-c4ccccc4)c3)c3ccc(-c4ccccc4)c4ccccc34)cc21 ; CC1(C)c2ccccc2-c2ccc(N(c3ccc4ccccc4c3)c3ccc4oc5ccccc5c4c3-c3cccc(-c4ccccc4)c3)cc21]

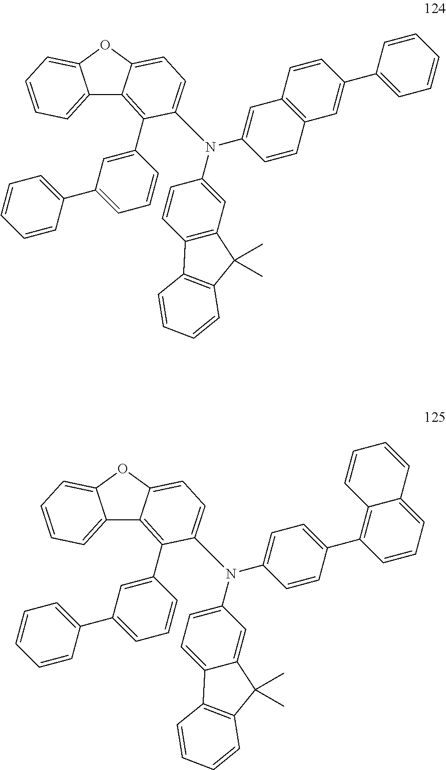 [SMILES: CC1(C)c2ccccc2-c2ccc(N(c3ccc4cc(-c5ccccc5)ccc4c3)c3ccc4oc5ccccc5c4c3-c3cccc(-c4ccccc4)c3)cc21 ; CC1(C)c2ccccc2-c2ccc(N(c3ccc(-c4cccc5ccccc45)cc3)c3ccc4oc5ccccc5c4c3-c3cccc(-c4ccccc4)c3)cc21]

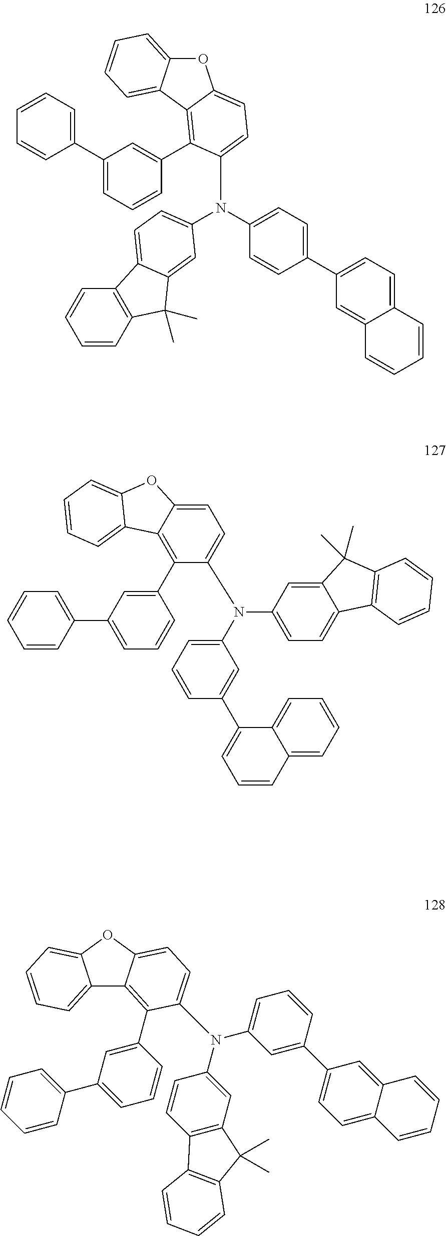 [SMILES: CC1(C)c2ccccc2-c2ccc(N(c3ccc(-c4ccc5ccccc5c4)cc3)c3ccc4oc5ccccc5c4c3-c3cccc(-c4ccccc4)c3)cc21 ; CC1(C)c2ccccc2-c2ccc(N(c3cccc(-c4cccc5ccccc45)c3)c3ccc4oc5ccccc5c4c3-c3cccc(-c4ccccc4)c3)cc21 ; CC1(C)c2ccccc2-c2ccc(N(c3cccc(-c4ccc5ccccc5c4)c3)c3ccc4oc5ccccc5c4c3-c3cccc(-c4ccccc4)c3)cc21]

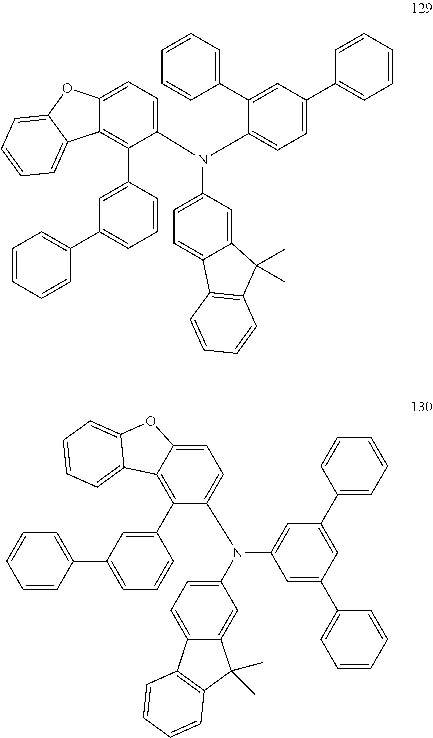 [SMILES: CC1(C)c2ccccc2-c2ccc(N(c3ccc(-c4ccccc4)cc3-c3ccccc3)c3ccc4oc5ccccc5c4c3-c3cccc(-c4ccccc4)c3)cc21 ; CC1(C)c2ccccc2-c2ccc(N(c3cc(-c4ccccc4)cc(-c4ccccc4)c3)c3ccc4oc5ccccc5c4c3-c3cccc(-c4ccccc4)c3)cc21]

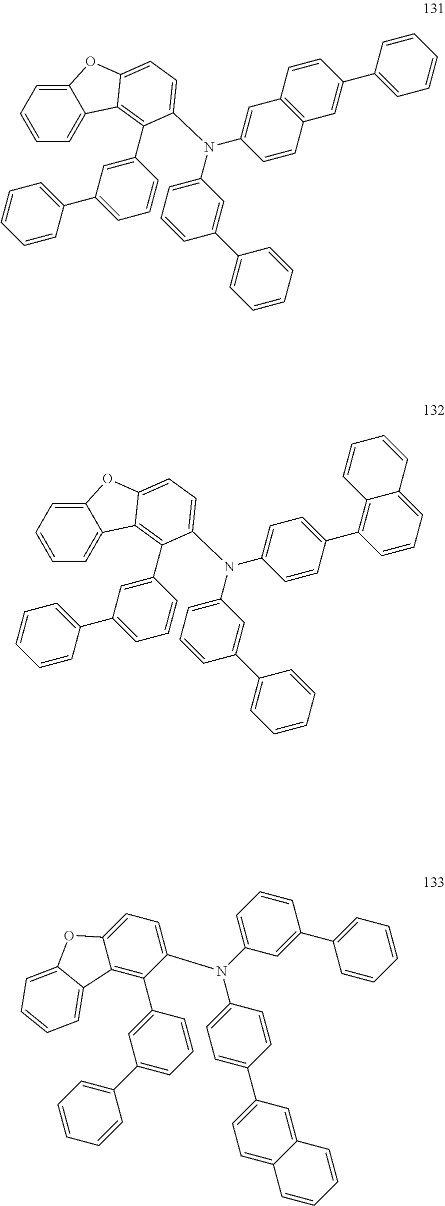 [SMILES: c1ccc(-c2cccc(-c3c(N(c4cccc(-c5ccccc5)c4)c4ccc5cc(-c6ccccc6)ccc5c4)ccc4oc5ccccc5c34)c2)cc1 ; c1ccc(-c2cccc(-c3c(N(c4ccc(-c5cccc6ccccc56)cc4)c4cccc(-c5ccccc5)c4)ccc4oc5ccccc5c34)c2)cc1 ; c1ccc(-c2cccc(-c3c(N(c4ccc(-c5ccc6ccccc6c5)cc4)c4cccc(-c5ccccc5)c4)ccc4oc5ccccc5c34)c2)cc1]

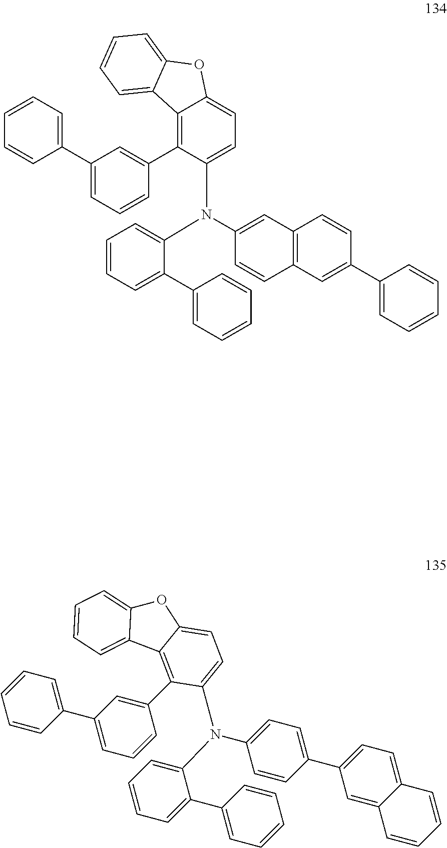 [SMILES: c1ccc(-c2cccc(-c3c(N(c4ccc5cc(-c6ccccc6)ccc5c4)c4ccccc4-c4ccccc4)ccc4oc5ccccc5c34)c2)cc1 ; c1ccc(-c2cccc(-c3c(N(c4ccc(-c5ccc6ccccc6c5)cc4)c4ccccc4-c4ccccc4)ccc4oc5ccccc5c34)c2)cc1]

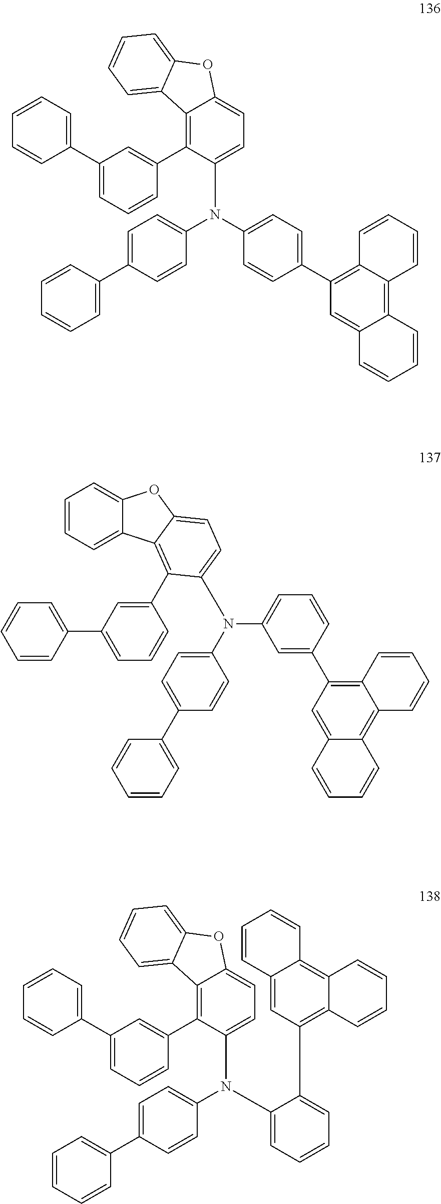 [SMILES: c1ccc(-c2ccc(N(c3ccc(-c4cc5ccccc5c5ccccc45)cc3)c3ccc4oc5ccccc5c4c3-c3cccc(-c4ccccc4)c3)cc2)cc1 ; c1ccc(-c2ccc(N(c3cccc(-c4cc5ccccc5c5ccccc45)c3)c3ccc4oc5ccccc5c4c3-c3cccc(-c4ccccc4)c3)cc2)cc1 ; c1ccc(-c2ccc(N(c3ccccc3-c3cc4ccccc4c4ccccc34)c3ccc4oc5ccccc5c4c3-c3cccc(-c4ccccc4)c3)cc2)cc1]

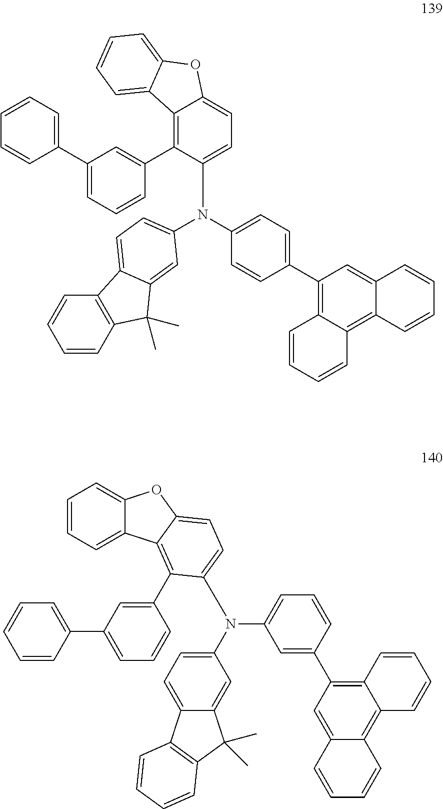 [SMILES: CC1(C)c2ccccc2-c2ccc(N(c3ccc(-c4cc5ccccc5c5ccccc45)cc3)c3ccc4oc5ccccc5c4c3-c3cccc(-c4ccccc4)c3)cc21 ; CC1(C)c2ccccc2-c2ccc(N(c3cccc(-c4cc5ccccc5c5ccccc45)c3)c3ccc4oc5ccccc5c4c3-c3cccc(-c4ccccc4)c3)cc21]

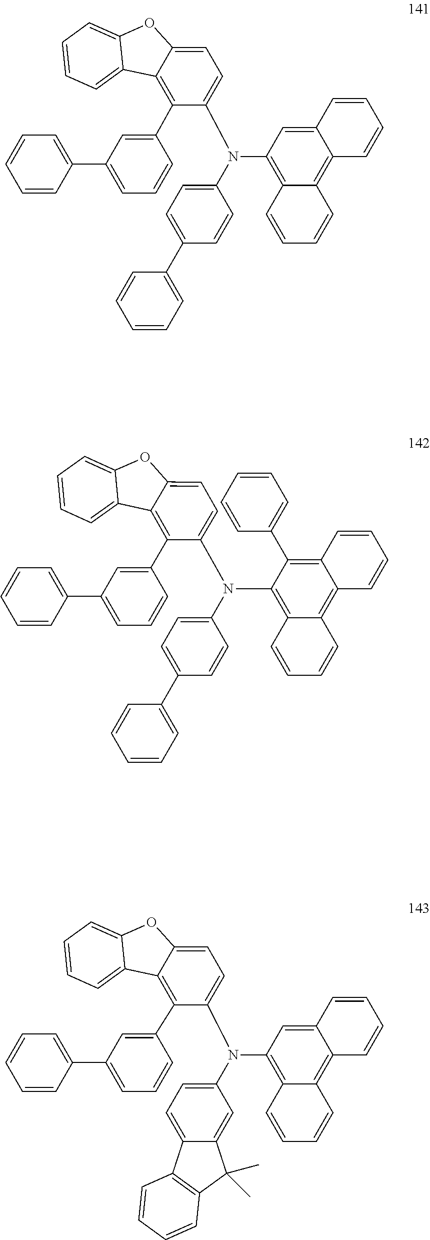 [SMILES: c1ccc(-c2ccc(N(c3ccc4oc5ccccc5c4c3-c3cccc(-c4ccccc4)c3)c3cc4ccccc4c4ccccc34)cc2)cc1 ; c1ccc(-c2ccc(N(c3ccc4oc5ccccc5c4c3-c3cccc(-c4ccccc4)c3)c3c(-c4ccccc4)c4ccccc4c4ccccc34)cc2)cc1 ; CC1(C)c2ccccc2-c2ccc(N(c3ccc4oc5ccccc5c4c3-c3cccc(-c4ccccc4)c3)c3cc4ccccc4c4ccccc34)cc21]

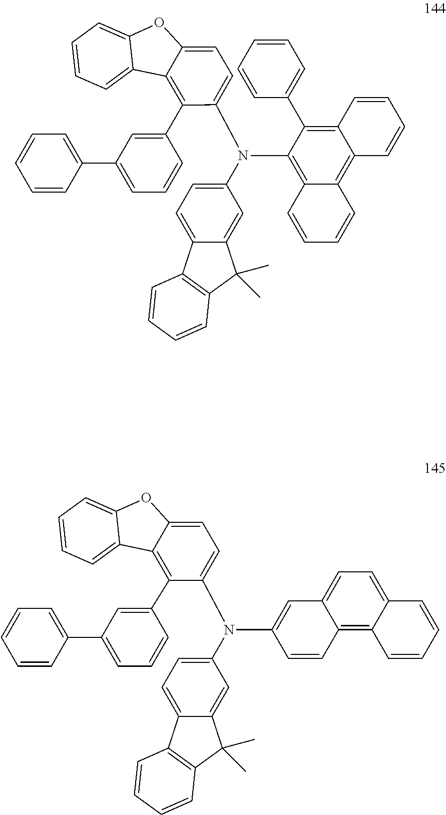 [SMILES: CC1(C)c2ccccc2-c2ccc(N(c3ccc4oc5ccccc5c4c3-c3cccc(-c4ccccc4)c3)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3cccc(-c4ccccc4)c3)cc21]

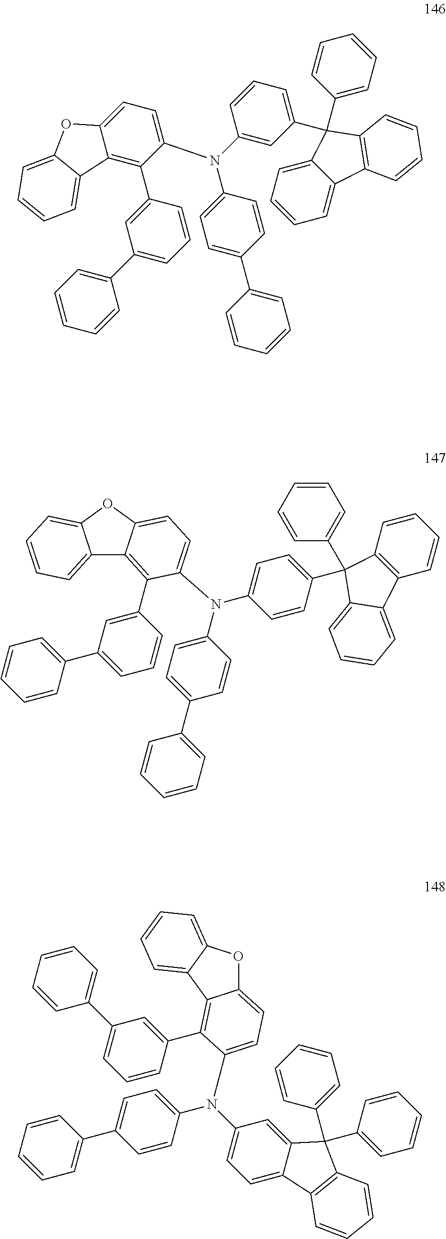 [SMILES: c1ccc(-c2ccc(N(c3cccc(C4(c5ccccc5)c5ccccc5-c5ccccc54)c3)c3ccc4oc5ccccc5c4c3-c3cccc(-c4ccccc4)c3)cc2)cc1 ; c1ccc(-c2ccc(N(c3ccc(C4(c5ccccc5)c5ccccc5-c5ccccc54)cc3)c3ccc4oc5ccccc5c4c3-c3cccc(-c4ccccc4)c3)cc2)cc1 ; c1ccc(-c2ccc(N(c3ccc4c(c3)C(c3ccccc3)(c3ccccc3)c3ccccc3-4)c3ccc4oc5ccccc5c4c3-c3cccc(-c4ccccc4)c3)cc2)cc1]

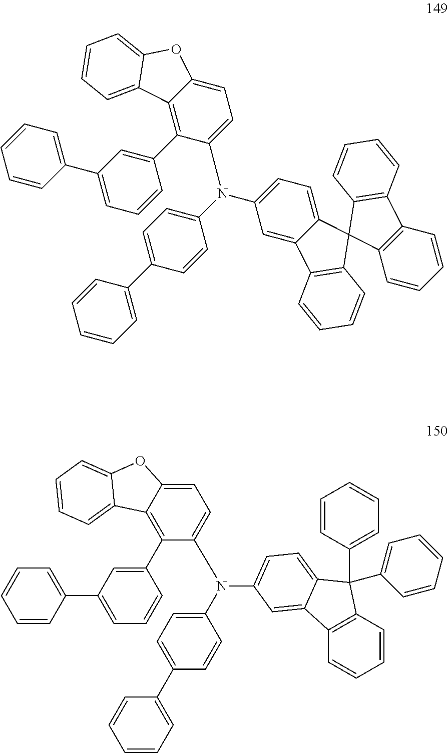 [SMILES: c1ccc(-c2ccc(N(c3ccc4c(c3)-c3ccccc3C43c4ccccc4-c4ccccc43)c3ccc4oc5ccccc5c4c3-c3cccc(-c4ccccc4)c3)cc2)cc1 ; c1ccc(-c2ccc(N(c3ccc4c(c3)-c3ccccc3C4(c3ccccc3)c3ccccc3)c3ccc4oc5ccccc5c4c3-c3cccc(-c4ccccc4)c3)cc2)cc1]

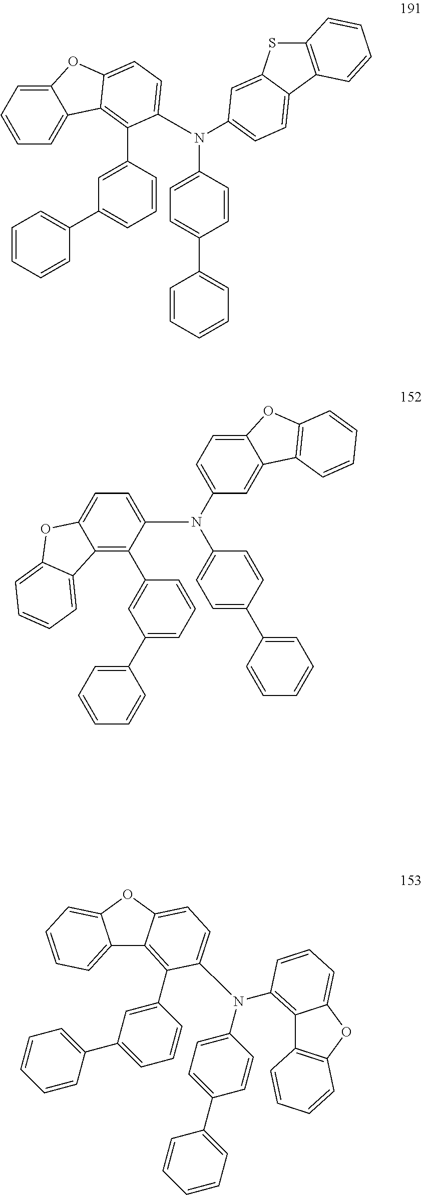 [SMILES: c1ccc(-c2ccc(N(c3ccc4c(c3)sc3ccccc34)c3ccc4oc5ccccc5c4c3-c3cccc(-c4ccccc4)c3)cc2)cc1 ; c1ccc(-c2ccc(N(c3ccc4oc5ccccc5c4c3)c3ccc4oc5ccccc5c4c3-c3cccc(-c4ccccc4)c3)cc2)cc1 ; c1ccc(-c2ccc(N(c3ccc4oc5ccccc5c4c3-c3cccc(-c4ccccc4)c3)c3cccc4oc5ccccc5c34)cc2)cc1]

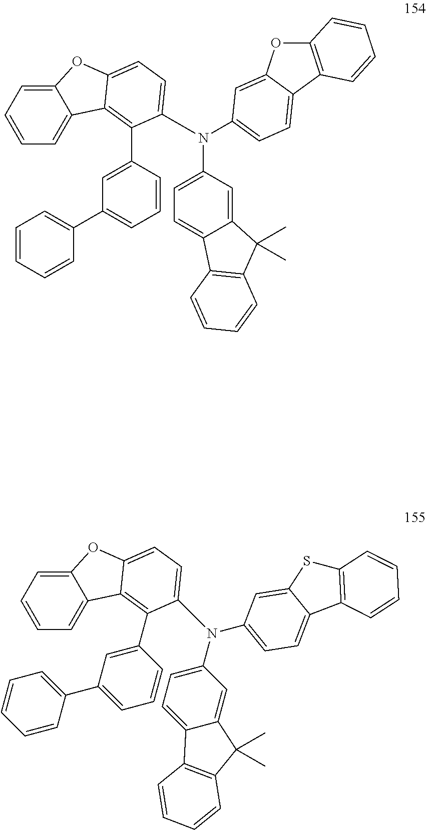 [SMILES: CC1(C)c2ccccc2-c2ccc(N(c3ccc4c(c3)oc3ccccc34)c3ccc4oc5ccccc5c4c3-c3cccc(-c4ccccc4)c3)cc21 ; CC1(C)c2ccccc2-c2ccc(N(c3ccc4c(c3)sc3ccccc34)c3ccc4oc5ccccc5c4c3-c3cccc(-c4ccccc4)c3)cc21]

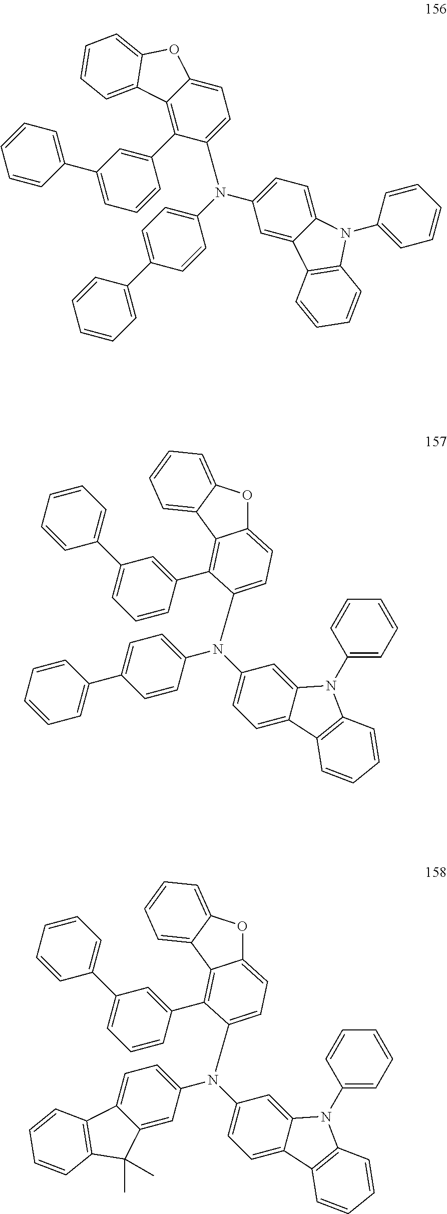 [SMILES: c1ccc(-c2ccc(N(c3ccc4c(c3)c3ccccc3n4-c3ccccc3)c3ccc4oc5ccccc5c4c3-c3cccc(-c4ccccc4)c3)cc2)cc1 ; c1ccc(-c2ccc(N(c3ccc4c5ccccc5n(-c5ccccc5)c4c3)c3ccc4oc5ccccc5c4c3-c3cccc(-c4ccccc4)c3)cc2)cc1 ; CC1(C)c2ccccc2-c2ccc(N(c3ccc4c5ccccc5n(-c5ccccc5)c4c3)c3ccc4oc5ccccc5c4c3-c3cccc(-c4ccccc4)c3)cc21]

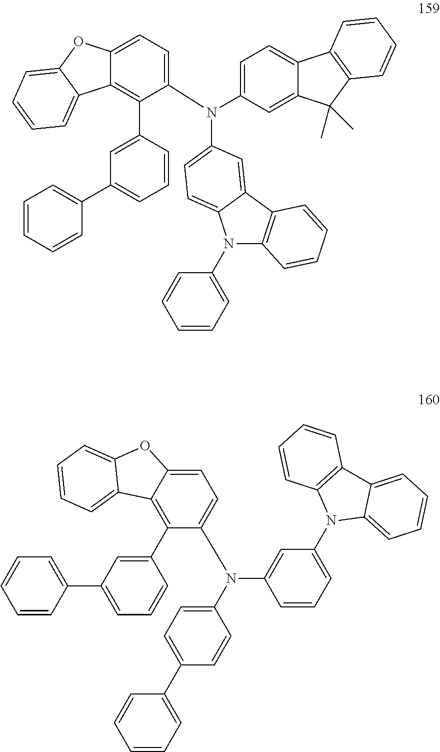 [SMILES: CC1(C)c2ccccc2-c2ccc(N(c3ccc4c(c3)c3ccccc3n4-c3ccccc3)c3ccc4oc5ccccc5c4c3-c3cccc(-c4ccccc4)c3)cc21 ; c1ccc(-c2ccc(N(c3cccc(-n4c5ccccc5c5ccccc54)c3)c3ccc4oc5ccccc5c4c3-c3cccc(-c4ccccc4)c3)cc2)cc1]

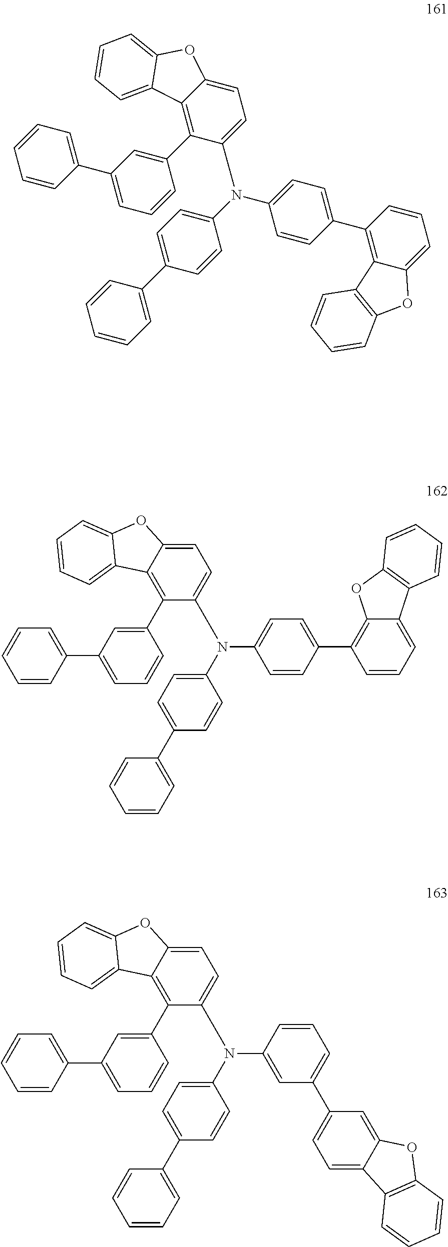 [SMILES: c1ccc(-c2ccc(N(c3ccc(-c4cccc5oc6ccccc6c45)cc3)c3ccc4oc5ccccc5c4c3-c3cccc(-c4ccccc4)c3)cc2)cc1 ; c1ccc(-c2ccc(N(c3ccc(-c4cccc5c4oc4ccccc45)cc3)c3ccc4oc5ccccc5c4c3-c3cccc(-c4ccccc4)c3)cc2)cc1 ; c1ccc(-c2ccc(N(c3cccc(-c4ccc5c(c4)oc4ccccc45)c3)c3ccc4oc5ccccc5c4c3-c3cccc(-c4ccccc4)c3)cc2)cc1]

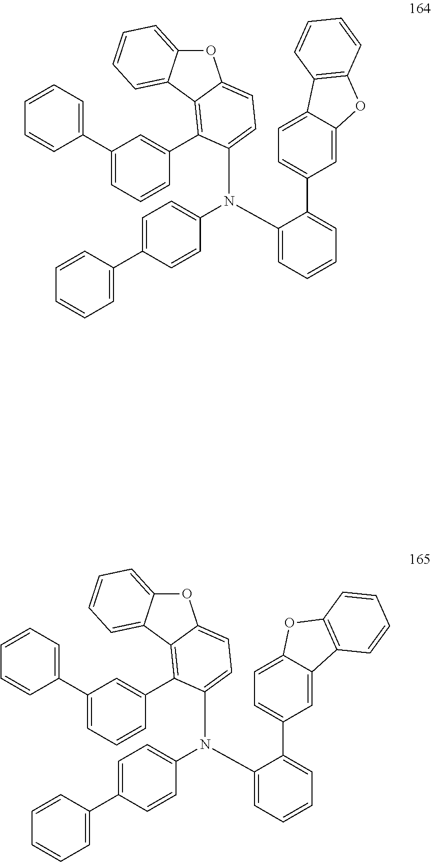 [SMILES: c1ccc(-c2ccc(N(c3ccccc3-c3ccc4c(c3)oc3ccccc34)c3ccc4oc5ccccc5c4c3-c3cccc(-c4ccccc4)c3)cc2)cc1 ; c1ccc(-c2ccc(N(c3ccccc3-c3ccc4oc5ccccc5c4c3)c3ccc4oc5ccccc5c4c3-c3cccc(-c4ccccc4)c3)cc2)cc1]

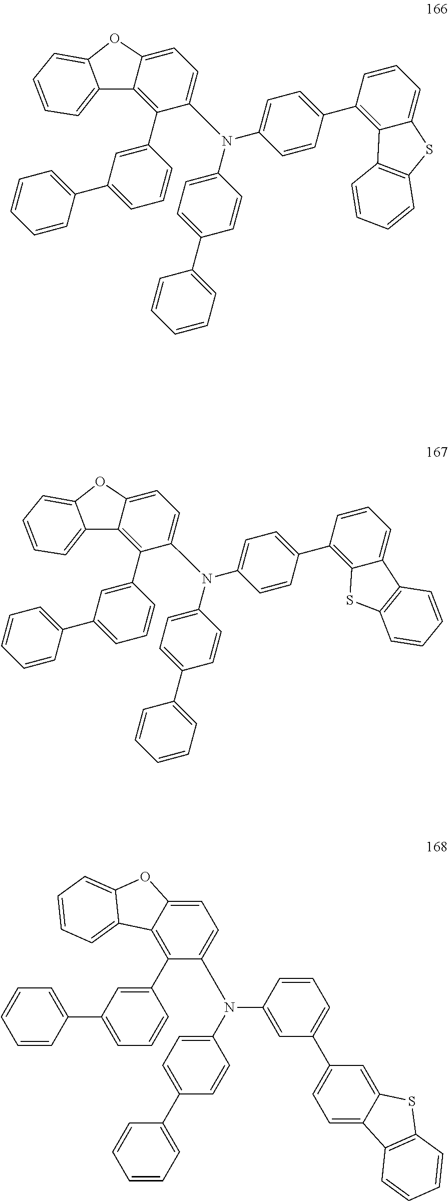 [SMILES: c1ccc(-c2ccc(N(c3ccc(-c4cccc5sc6ccccc6c45)cc3)c3ccc4oc5ccccc5c4c3-c3cccc(-c4ccccc4)c3)cc2)cc1 ; c1ccc(-c2ccc(N(c3ccc(-c4cccc5c4sc4ccccc45)cc3)c3ccc4oc5ccccc5c4c3-c3cccc(-c4ccccc4)c3)cc2)cc1 ; c1ccc(-c2ccc(N(c3cccc(-c4ccc5c(c4)sc4ccccc45)c3)c3ccc4oc5ccccc5c4c3-c3cccc(-c4ccccc4)c3)cc2)cc1]

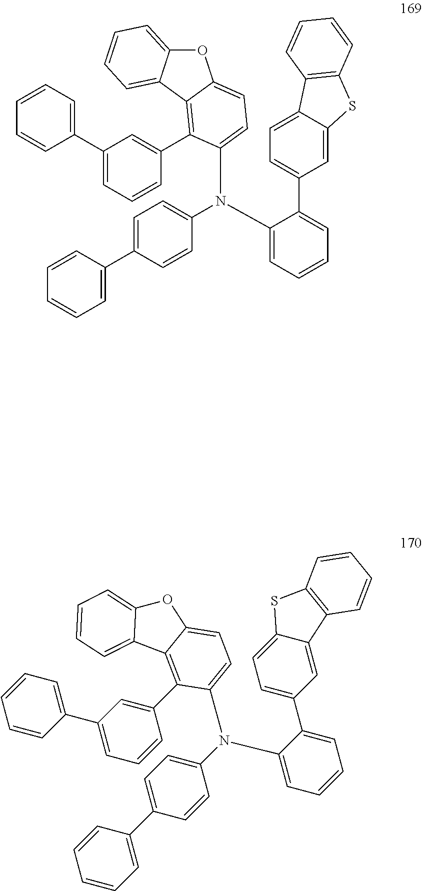 [SMILES: c1ccc(-c2ccc(N(c3ccccc3-c3ccc4c(c3)sc3ccccc34)c3ccc4oc5ccccc5c4c3-c3cccc(-c4ccccc4)c3)cc2)cc1 ; c1ccc(-c2ccc(N(c3ccccc3-c3ccc4sc5ccccc5c4c3)c3ccc4oc5ccccc5c4c3-c3cccc(-c4ccccc4)c3)cc2)cc1]

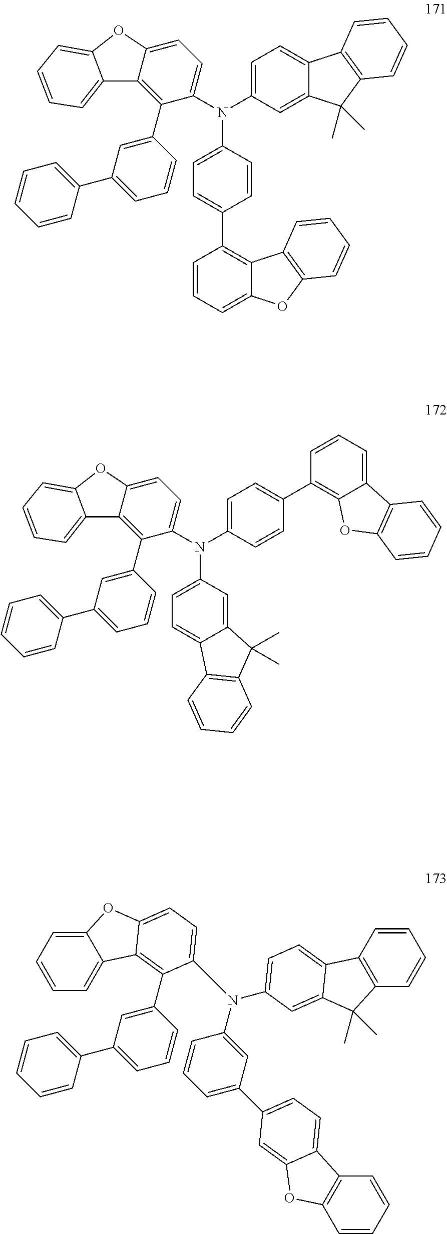 [SMILES: CC1(C)c2ccccc2-c2ccc(N(c3ccc(-c4cccc5oc6ccccc6c45)cc3)c3ccc4oc5ccccc5c4c3-c3cccc(-c4ccccc4)c3)cc21 ; CC1(C)c2ccccc2-c2ccc(N(c3ccc(-c4cccc5c4oc4ccccc45)cc3)c3ccc4oc5ccccc5c4c3-c3cccc(-c4ccccc4)c3)cc21 ; CC1(C)c2ccccc2-c2ccc(N(c3cccc(-c4ccc5c(c4)oc4ccccc45)c3)c3ccc4oc5ccccc5c4c3-c3cccc(-c4ccccc4)c3)cc21]

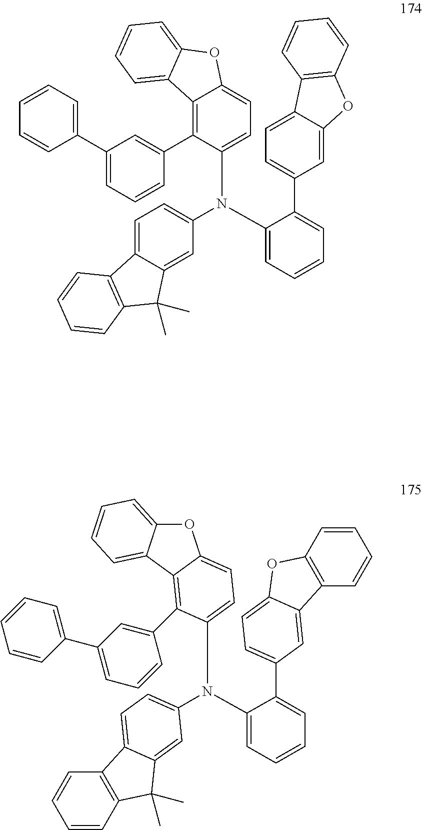 [SMILES: CC1(C)c2ccccc2-c2ccc(N(c3ccccc3-c3ccc4c(c3)oc3ccccc34)c3ccc4oc5ccccc5c4c3-c3cccc(-c4ccccc4)c3)cc21 ; CC1(C)c2ccccc2-c2ccc(N(c3ccccc3-c3ccc4oc5ccccc5c4c3)c3ccc4oc5ccccc5c4c3-c3cccc(-c4ccccc4)c3)cc21]

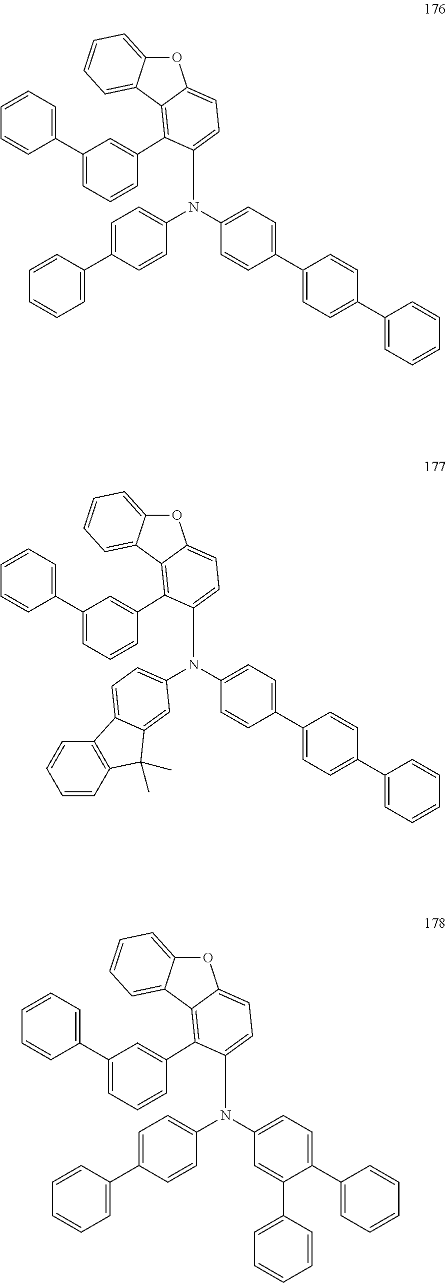 [SMILES: c1ccc(-c2ccc(-c3ccc(N(c4ccc(-c5ccccc5)cc4)c4ccc5oc6ccccc6c5c4-c4cccc(-c5ccccc5)c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc(-c4ccccc4)c3)cc21 ; c1ccc(-c2ccc(N(c3ccc(-c4ccccc4)c(-c4ccccc4)c3)c3ccc4oc5ccccc5c4c3-c3cccc(-c4ccccc4)c3)cc2)cc1]

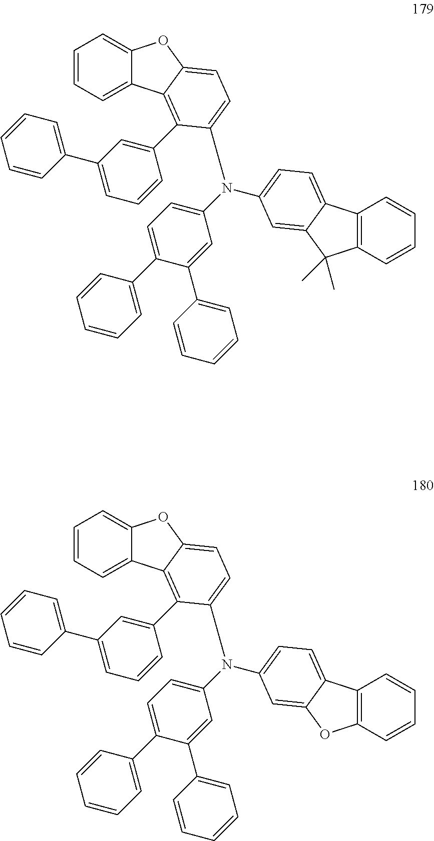 [SMILES: CC1(C)c2ccccc2-c2ccc(N(c3ccc(-c4ccccc4)c(-c4ccccc4)c3)c3ccc4oc5ccccc5c4c3-c3cccc(-c4ccccc4)c3)cc21 ; c1ccc(-c2cccc(-c3c(N(c4ccc(-c5ccccc5)c(-c5ccccc5)c4)c4ccc5c(c4)oc4ccccc45)ccc4oc5ccccc5c34)c2)cc1]

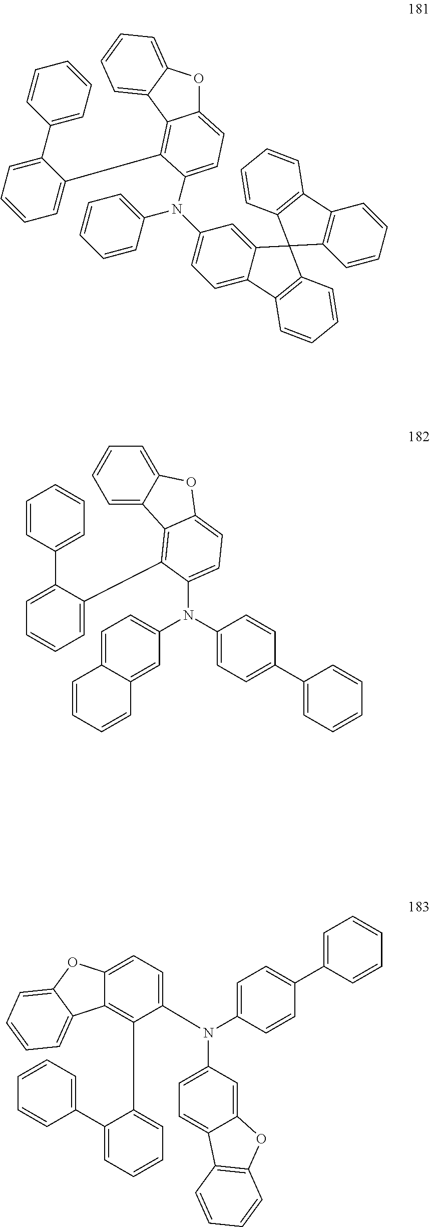 [SMILES: c1ccc(-c2ccccc2-c2c(N(c3ccccc3)c3ccc4c(c3)C3(c5ccccc5-c5ccccc53)c3ccccc3-4)ccc3oc4ccccc4c23)cc1 ; c1ccc(-c2ccc(N(c3ccc4ccccc4c3)c3ccc4oc5ccccc5c4c3-c3ccccc3-c3ccccc3)cc2)cc1 ; c1ccc(-c2ccc(N(c3ccc4c(c3)oc3ccccc34)c3ccc4oc5ccccc5c4c3-c3ccccc3-c3ccccc3)cc2)cc1]

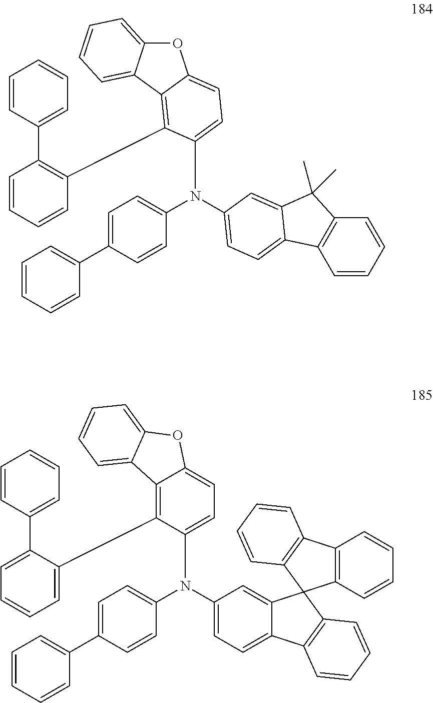 [SMILES: CC1(C)c2ccccc2-c2ccc(N(c3ccc(-c4ccccc4)cc3)c3ccc4oc5ccccc5c4c3-c3ccccc3-c3ccccc3)cc21 ; c1ccc(-c2ccc(N(c3ccc4c(c3)C3(c5ccccc5-c5ccccc53)c3ccccc3-4)c3ccc4oc5ccccc5c4c3-c3ccccc3-c3ccccc3)cc2)cc1]

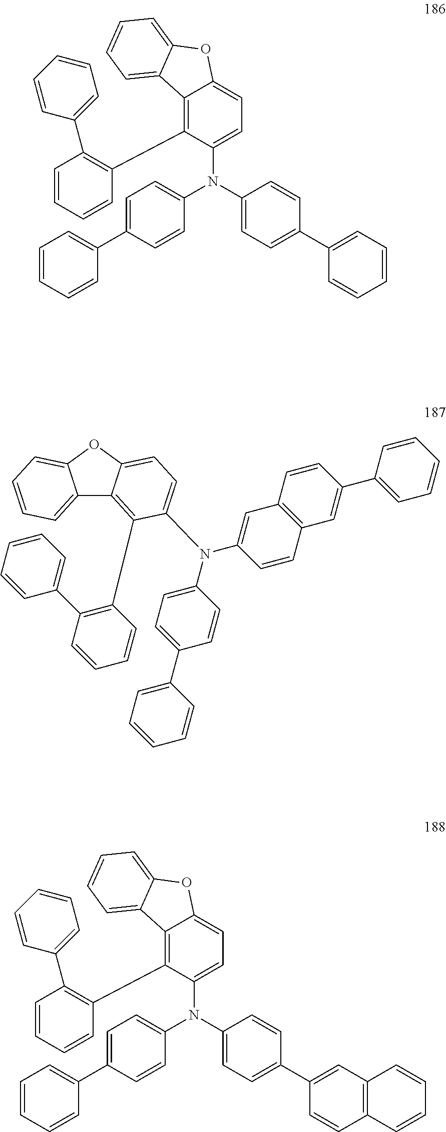 [SMILES: c1ccc(-c2ccc(N(c3ccc(-c4ccccc4)cc3)c3ccc4oc5ccccc5c4c3-c3ccccc3-c3ccccc3)cc2)cc1 ; c1ccc(-c2ccc(N(c3ccc4cc(-c5ccccc5)ccc4c3)c3ccc4oc5ccccc5c4c3-c3ccccc3-c3ccccc3)cc2)cc1 ; c1ccc(-c2ccc(N(c3ccc(-c4ccc5ccccc5c4)cc3)c3ccc4oc5ccccc5c4c3-c3ccccc3-c3ccccc3)cc2)cc1]

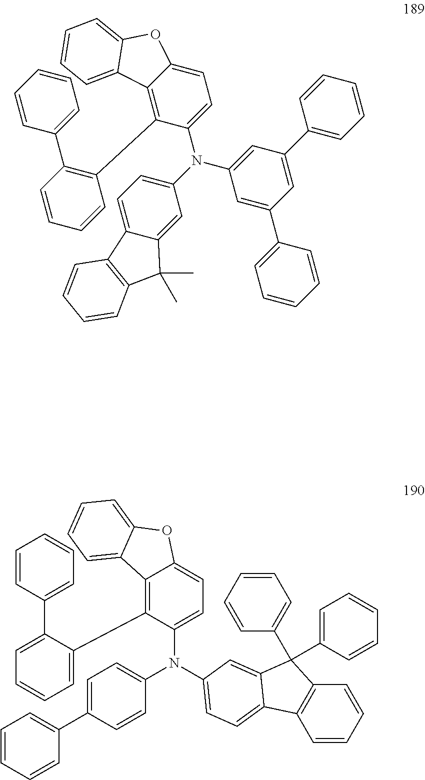 [SMILES: CC1(C)c2ccccc2-c2ccc(N(c3cc(-c4ccccc4)cc(-c4ccccc4)c3)c3ccc4oc5ccccc5c4c3-c3ccccc3-c3ccccc3)cc21 ; c1ccc(-c2ccc(N(c3ccc4c(c3)C(c3ccccc3)(c3ccccc3)c3ccccc3-4)c3ccc4oc5ccccc5c4c3-c3ccccc3-c3ccccc3)cc2)cc1]

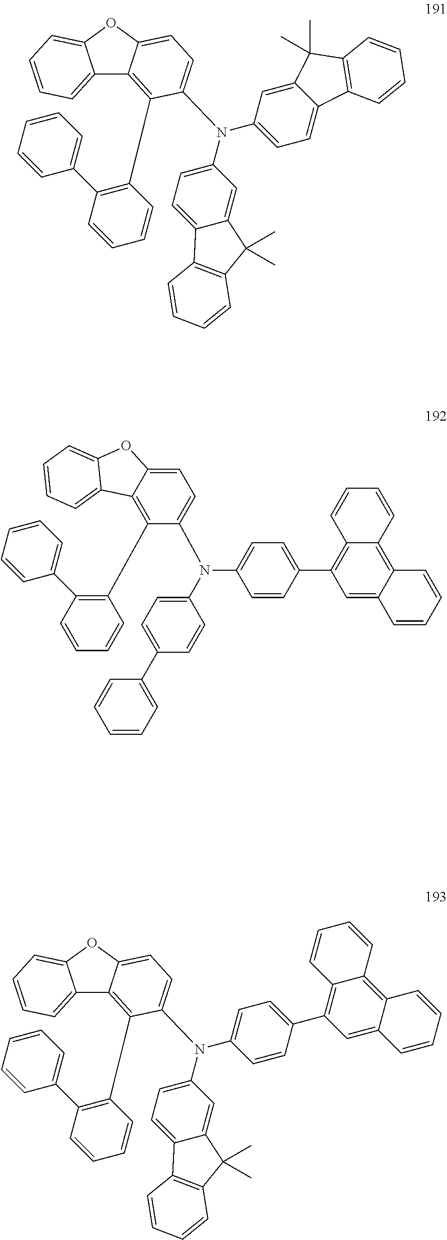 [SMILES: CC1(C)c2ccccc2-c2ccc(N(c3ccc4c(c3)C(C)(C)c3ccccc3-4)c3ccc4oc5ccccc5c4c3-c3ccccc3-c3ccccc3)cc21 ; c1ccc(-c2ccc(N(c3ccc(-c4cc5ccccc5c5ccccc45)cc3)c3ccc4oc5ccccc5c4c3-c3ccccc3-c3ccccc3)cc2)cc1 ; CC1(C)c2ccccc2-c2ccc(N(c3ccc(-c4cc5ccccc5c5ccccc45)cc3)c3ccc4oc5ccccc5c4c3-c3ccccc3-c3ccccc3)cc21]

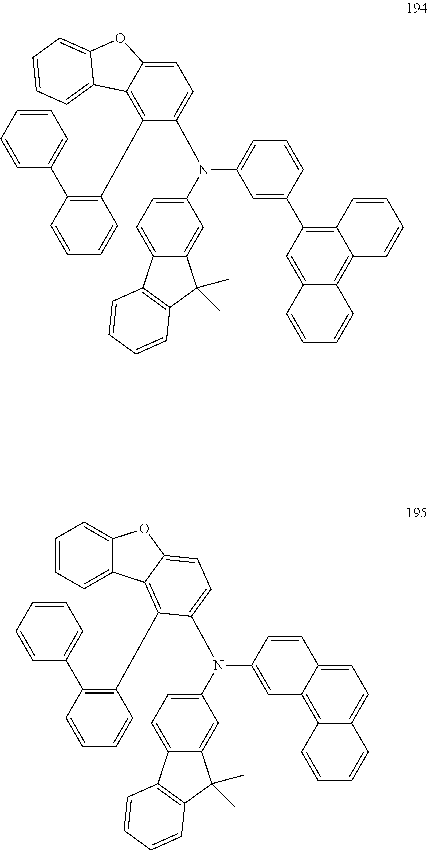 [SMILES: CC1(C)c2ccccc2-c2ccc(N(c3cccc(-c4cc5ccccc5c5ccccc45)c3)c3ccc4oc5ccccc5c4c3-c3ccccc3-c3ccccc3)cc21 ; CC1(C)c2ccccc2-c2ccc(N(c3ccc4ccc5ccccc5c4c3)c3ccc4oc5ccccc5c4c3-c3ccccc3-c3ccccc3)cc21]

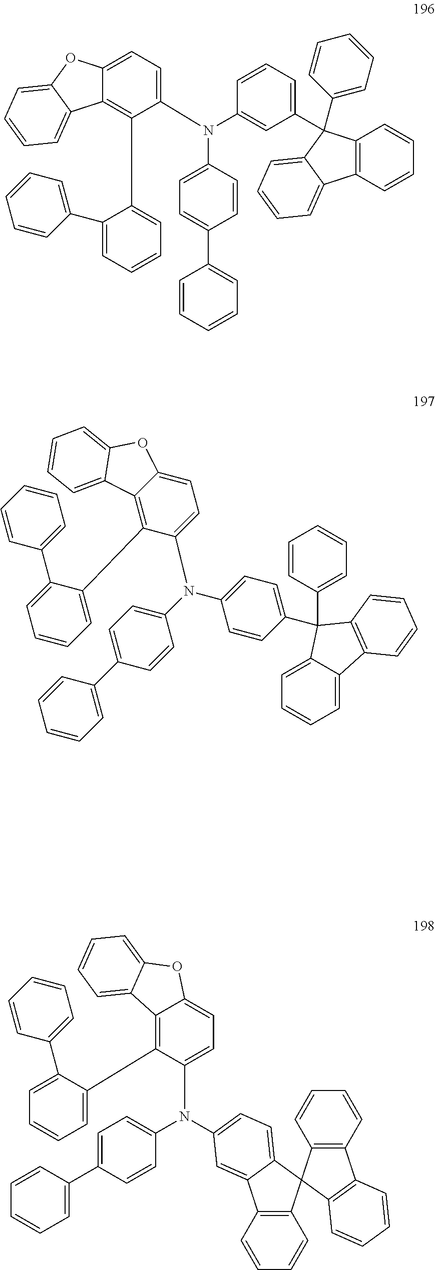 [SMILES: c1ccc(-c2ccc(N(c3cccc(C4(c5ccccc5)c5ccccc5-c5ccccc54)c3)c3ccc4oc5ccccc5c4c3-c3ccccc3-c3ccccc3)cc2)cc1 ; c1ccc(-c2ccc(N(c3ccc(C4(c5ccccc5)c5ccccc5-c5ccccc54)cc3)c3ccc4oc5ccccc5c4c3-c3ccccc3-c3ccccc3)cc2)cc1 ; c1ccc(-c2ccc(N(c3ccc4c(c3)-c3ccccc3C43c4ccccc4-c4ccccc43)c3ccc4oc5ccccc5c4c3-c3ccccc3-c3ccccc3)cc2)cc1]

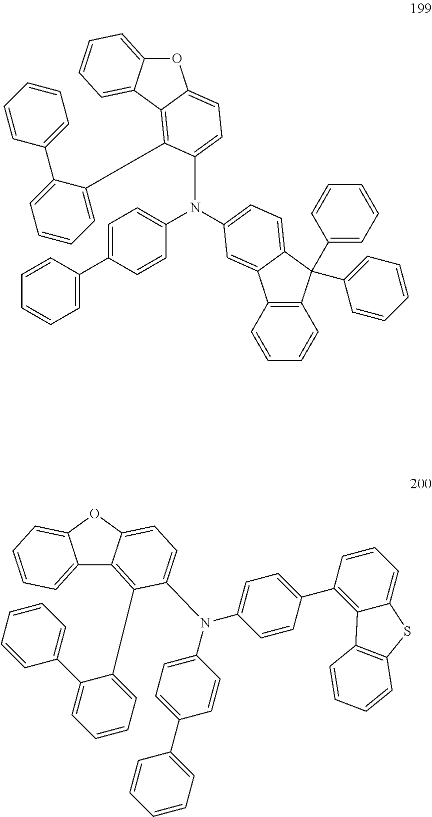 [SMILES: c1ccc(-c2ccc(N(c3ccc4c(c3)-c3ccccc3C4(c3ccccc3)c3ccccc3)c3ccc4oc5ccccc5c4c3-c3ccccc3-c3ccccc3)cc2)cc1 ; c1ccc(-c2ccc(N(c3ccc(-c4cccc5sc6ccccc6c45)cc3)c3ccc4oc5ccccc5c4c3-c3ccccc3-c3ccccc3)cc2)cc1]

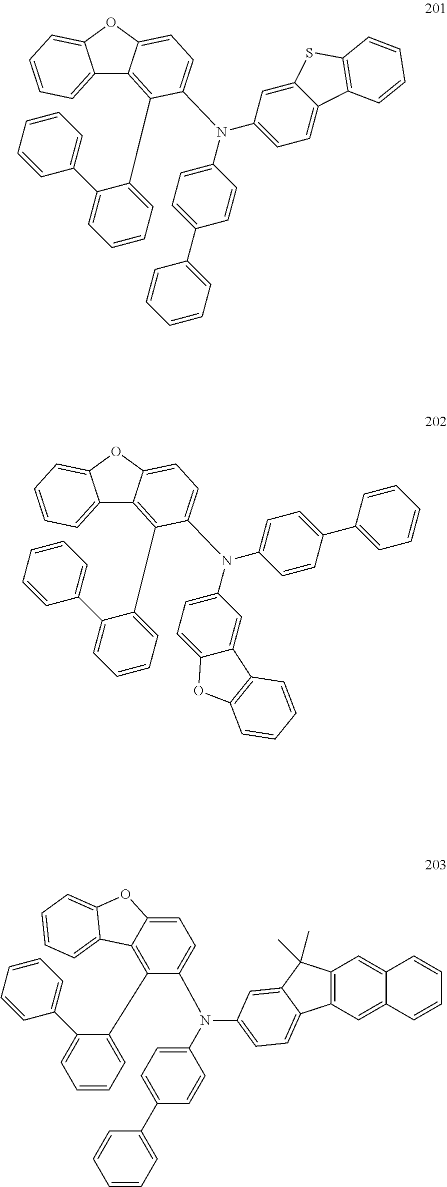 [SMILES: c1ccc(-c2ccc(N(c3ccc4c(c3)sc3ccccc34)c3ccc4oc5ccccc5c4c3-c3ccccc3-c3ccccc3)cc2)cc1 ; c1ccc(-c2ccc(N(c3ccc4oc5ccccc5c4c3)c3ccc4oc5ccccc5c4c3-c3ccccc3-c3ccccc3)cc2)cc1 ; CC1(C)c2cc(N(c3ccc(-c4ccccc4)cc3)c3ccc4oc5ccccc5c4c3-c3ccccc3-c3ccccc3)ccc2-c2cc3ccccc3cc21]

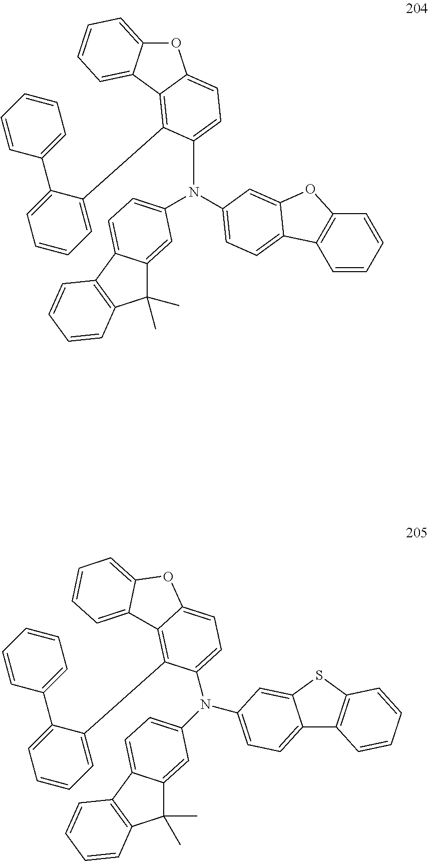 [SMILES: CC1(C)c2ccccc2-c2ccc(N(c3ccc4c(c3)oc3ccccc34)c3ccc4oc5ccccc5c4c3-c3ccccc3-c3ccccc3)cc21 ; CC1(C)c2ccccc2-c2ccc(N(c3ccc4c(c3)sc3ccccc34)c3ccc4oc5ccccc5c4c3-c3ccccc3-c3ccccc3)cc21]

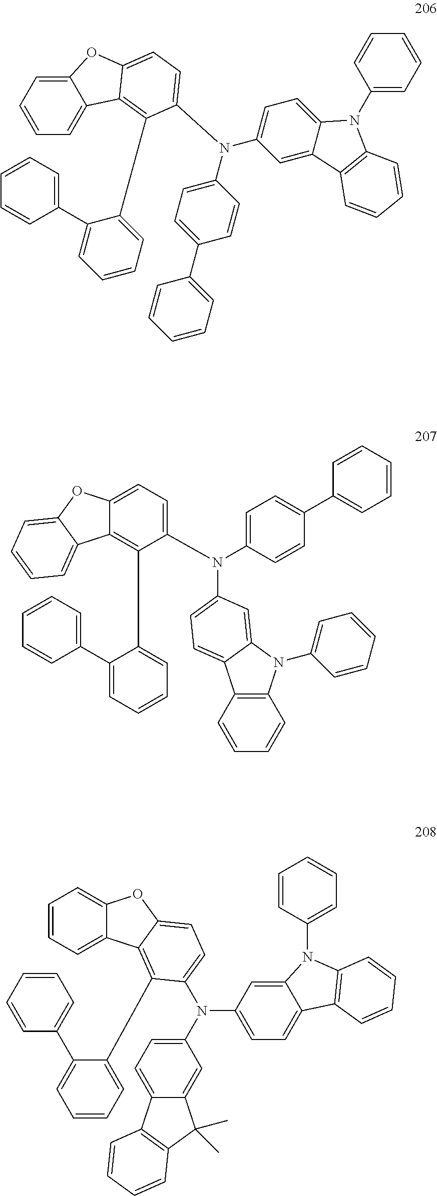 [SMILES: c1ccc(-c2ccc(N(c3ccc4c(c3)c3ccccc3n4-c3ccccc3)c3ccc4oc5ccccc5c4c3-c3ccccc3-c3ccccc3)cc2)cc1 ; c1ccc(-c2ccc(N(c3ccc4c5ccccc5n(-c5ccccc5)c4c3)c3ccc4oc5ccccc5c4c3-c3ccccc3-c3ccccc3)cc2)cc1 ; CC1(C)c2ccccc2-c2ccc(N(c3ccc4c5ccccc5n(-c5ccccc5)c4c3)c3ccc4oc5ccccc5c4c3-c3ccccc3-c3ccccc3)cc21]

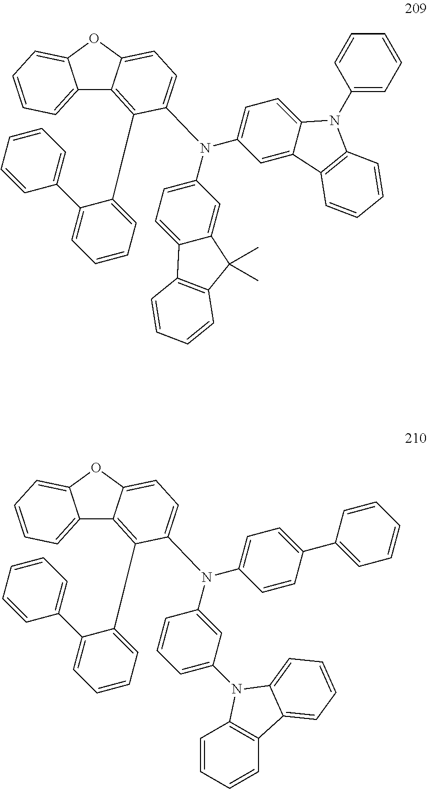 [SMILES: CC1(C)c2ccccc2-c2ccc(N(c3ccc4c(c3)c3ccccc3n4-c3ccccc3)c3ccc4oc5ccccc5c4c3-c3ccccc3-c3ccccc3)cc21 ; c1ccc(-c2ccc(N(c3cccc(-n4c5ccccc5c5ccccc54)c3)c3ccc4oc5ccccc5c4c3-c3ccccc3-c3ccccc3)cc2)cc1]

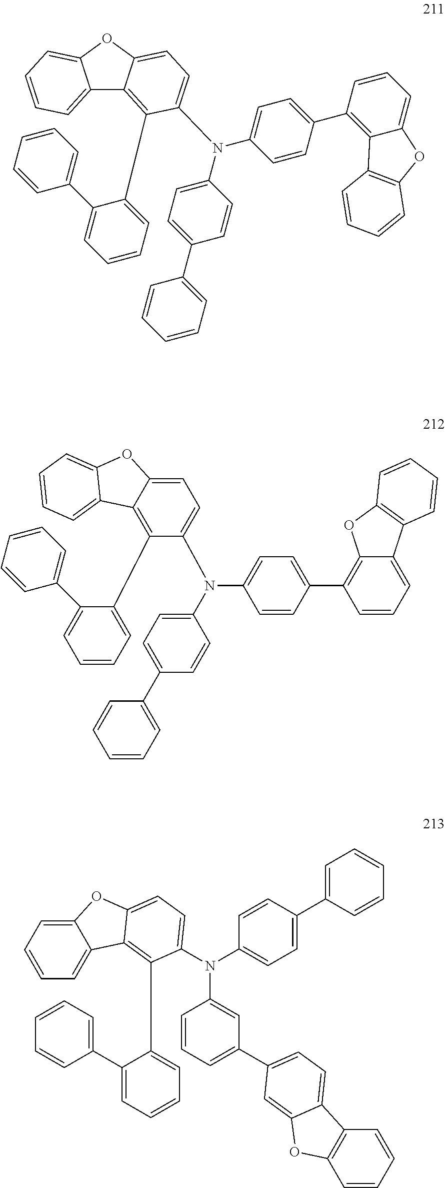 [SMILES: c1ccc(-c2ccc(N(c3ccc(-c4cccc5oc6ccccc6c45)cc3)c3ccc4oc5ccccc5c4c3-c3ccccc3-c3ccccc3)cc2)cc1 ; c1ccc(-c2ccc(N(c3ccc(-c4cccc5c4oc4ccccc45)cc3)c3ccc4oc5ccccc5c4c3-c3ccccc3-c3ccccc3)cc2)cc1 ; c1ccc(-c2ccc(N(c3cccc(-c4ccc5c(c4)oc4ccccc45)c3)c3ccc4oc5ccccc5c4c3-c3ccccc3-c3ccccc3)cc2)cc1]

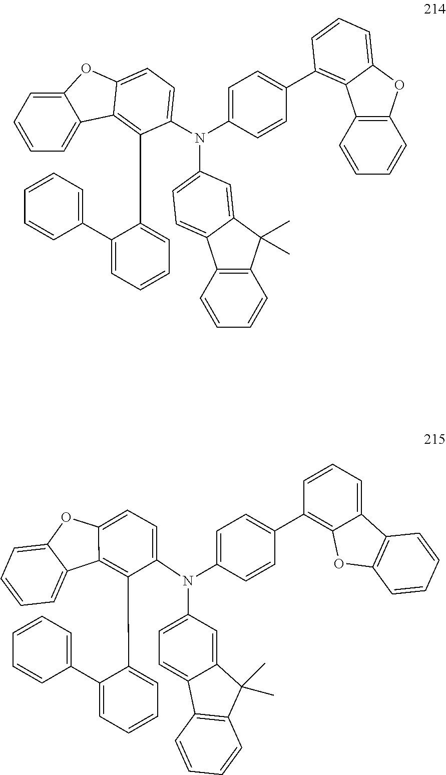 [SMILES: CC1(C)c2ccccc2-c2ccc(N(c3ccc(-c4cccc5oc6ccccc6c45)cc3)c3ccc4oc5ccccc5c4c3-c3ccccc3-c3ccccc3)cc21 ; CC1(C)c2ccccc2-c2ccc(N(c3ccc(-c4cccc5c4oc4ccccc45)cc3)c3ccc4oc5ccccc5c4c3-c3ccccc3-c3ccccc3)cc21]

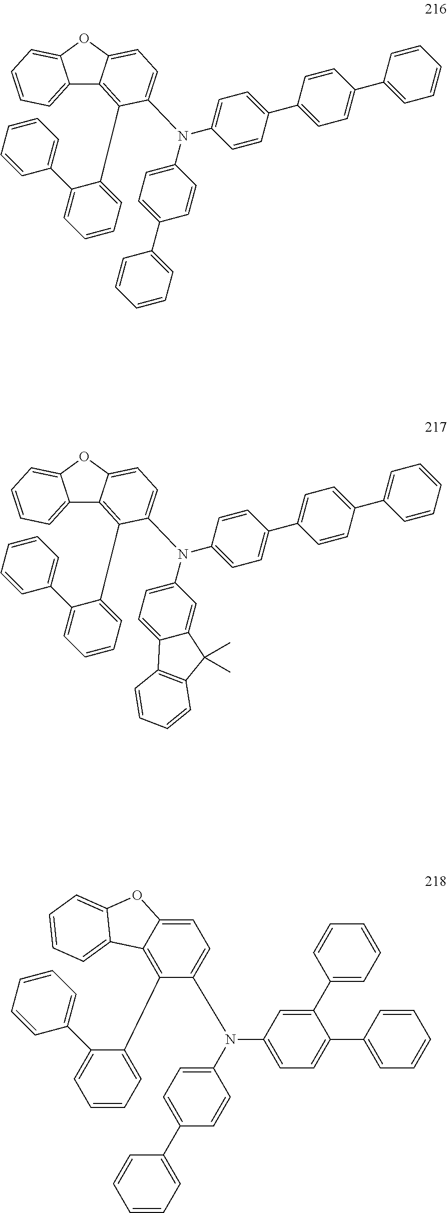 [SMILES: c1ccc(-c2ccc(-c3ccc(N(c4ccc(-c5ccccc5)cc4)c4ccc5oc6ccccc6c5c4-c4ccccc4-c4ccccc4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccccc3-c3ccccc3)cc21 ; c1ccc(-c2ccc(N(c3ccc(-c4ccccc4)c(-c4ccccc4)c3)c3ccc4oc5ccccc5c4c3-c3ccccc3-c3ccccc3)cc2)cc1]

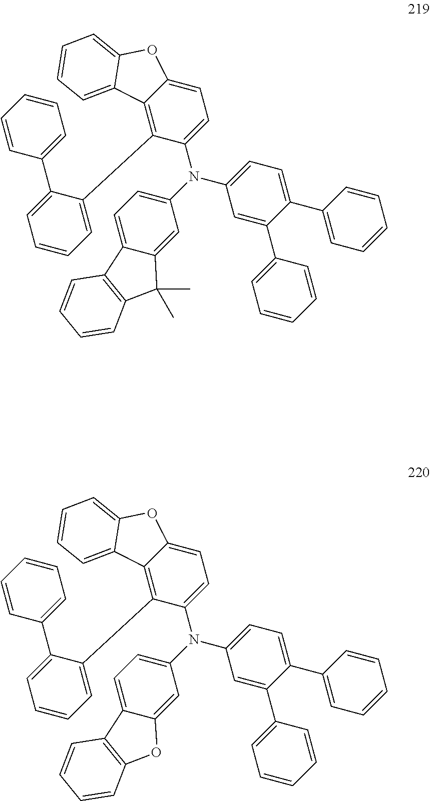 [SMILES: CC1(C)c2ccccc2-c2ccc(N(c3ccc(-c4ccccc4)c(-c4ccccc4)c3)c3ccc4oc5ccccc5c4c3-c3ccccc3-c3ccccc3)cc21 ; c1ccc(-c2ccc(N(c3ccc4c(c3)oc3ccccc34)c3ccc4oc5ccccc5c4c3-c3ccccc3-c3ccccc3)cc2-c2ccccc2)cc1]

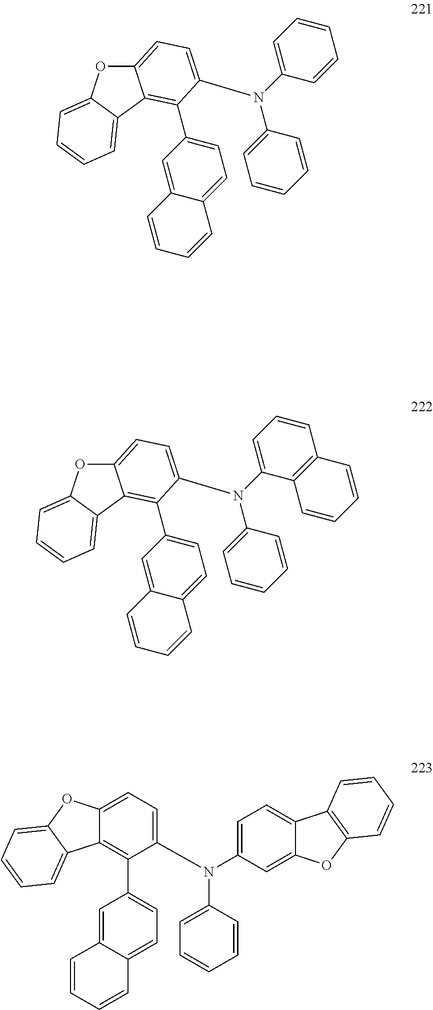 [SMILES: c1ccc(N(c2ccccc2)c2ccc3oc4ccccc4c3c2-c2ccc3ccccc3c2)cc1 ; c1ccc(N(c2ccc3oc4ccccc4c3c2-c2ccc3ccccc3c2)c2cccc3ccccc23)cc1 ; c1ccc(N(c2ccc3c(c2)oc2ccccc23)c2ccc3oc4ccccc4c3c2-c2ccc3ccccc3c2)cc1]

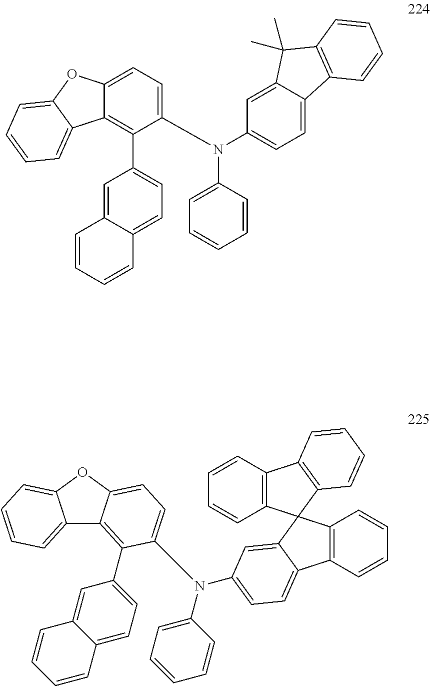 [SMILES: CC1(C)c2ccccc2-c2ccc(N(c3ccccc3)c3ccc4oc5ccccc5c4c3-c3ccc4ccccc4c3)cc21 ; c1ccc(N(c2ccc3c(c2)C2(c4ccccc4-c4ccccc42)c2ccccc2-3)c2ccc3oc4ccccc4c3c2-c2ccc3ccccc3c2)cc1]

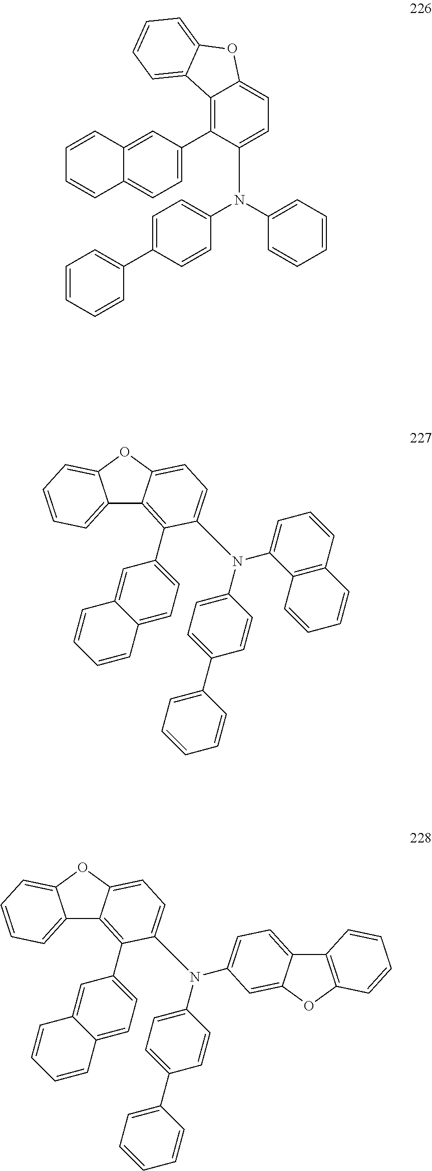 [SMILES: c1ccc(-c2ccc(N(c3ccccc3)c3ccc4oc5ccccc5c4c3-c3ccc4ccccc4c3)cc2)cc1 ; c1ccc(-c2ccc(N(c3ccc4oc5ccccc5c4c3-c3ccc4ccccc4c3)c3cccc4ccccc34)cc2)cc1 ; c1ccc(-c2ccc(N(c3ccc4c(c3)oc3ccccc34)c3ccc4oc5ccccc5c4c3-c3ccc4ccccc4c3)cc2)cc1]

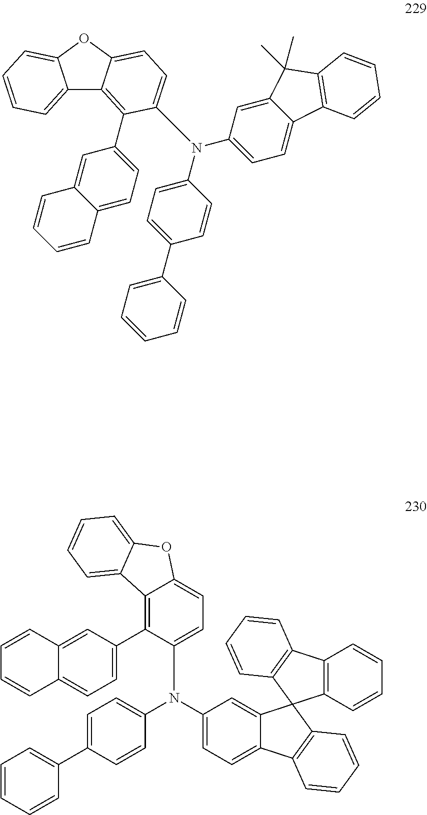 [SMILES: CC1(C)c2ccccc2-c2ccc(N(c3ccc(-c4ccccc4)cc3)c3ccc4oc5ccccc5c4c3-c3ccc4ccccc4c3)cc21 ; c1ccc(-c2ccc(N(c3ccc4c(c3)C3(c5ccccc5-c5ccccc53)c3ccccc3-4)c3ccc4oc5ccccc5c4c3-c3ccc4ccccc4c3)cc2)cc1]

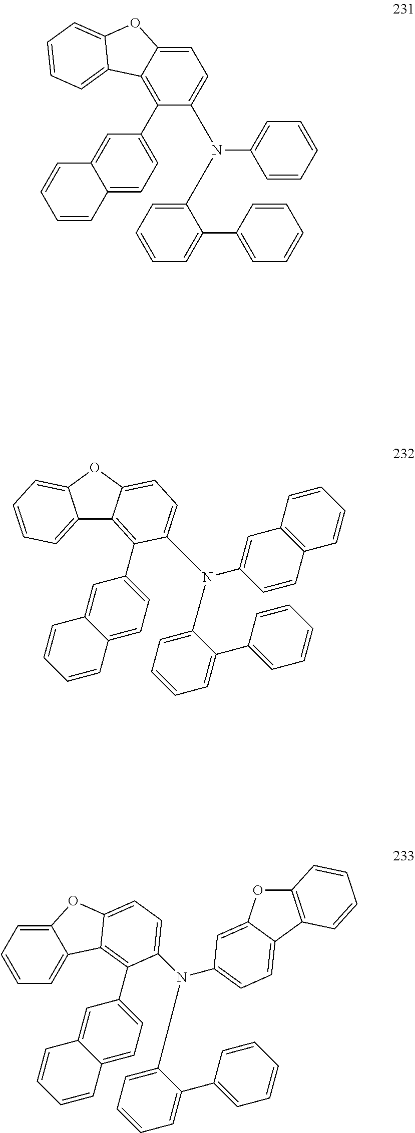 [SMILES: c1ccc(-c2ccccc2N(c2ccccc2)c2ccc3oc4ccccc4c3c2-c2ccc3ccccc3c2)cc1 ; c1ccc(-c2ccccc2N(c2ccc3ccccc3c2)c2ccc3oc4ccccc4c3c2-c2ccc3ccccc3c2)cc1 ; c1ccc(-c2ccccc2N(c2ccc3c(c2)oc2ccccc23)c2ccc3oc4ccccc4c3c2-c2ccc3ccccc3c2)cc1]

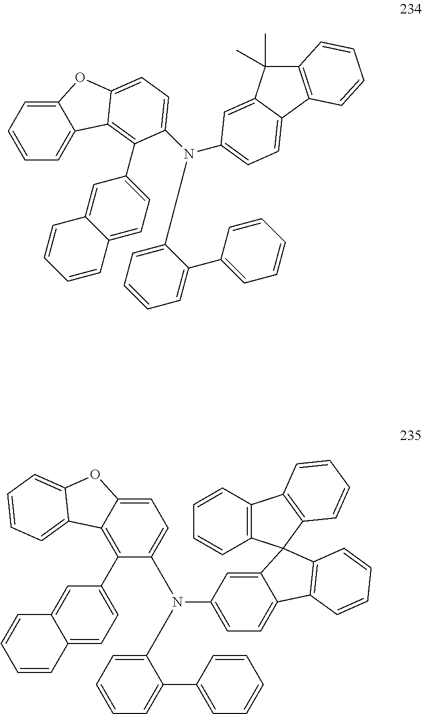 [SMILES: CC1(C)c2ccccc2-c2ccc(N(c3ccccc3-c3ccccc3)c3ccc4oc5ccccc5c4c3-c3ccc4ccccc4c3)cc21 ; c1ccc(-c2ccccc2N(c2ccc3c(c2)C2(c4ccccc4-c4ccccc42)c2ccccc2-3)c2ccc3oc4ccccc4c3c2-c2ccc3ccccc3c2)cc1]

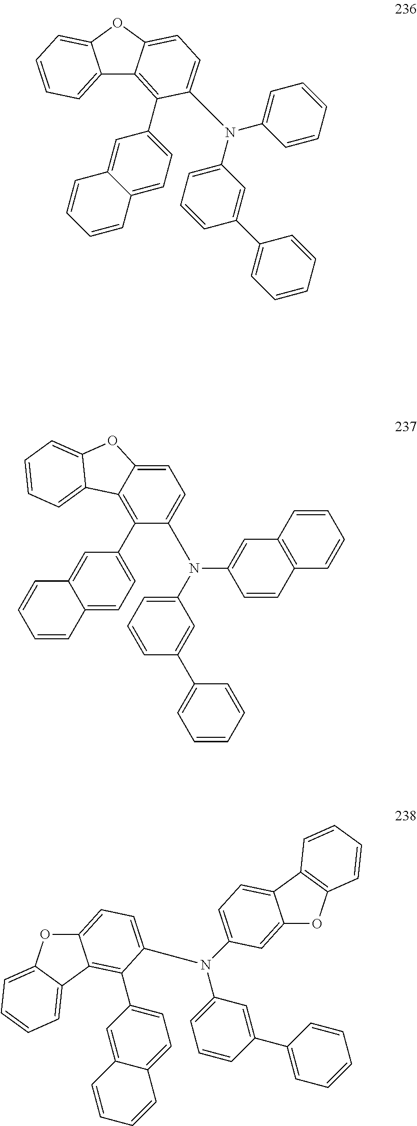 [SMILES: c1ccc(-c2cccc(N(c3ccccc3)c3ccc4oc5ccccc5c4c3-c3ccc4ccccc4c3)c2)cc1 ; c1ccc(-c2cccc(N(c3ccc4ccccc4c3)c3ccc4oc5ccccc5c4c3-c3ccc4ccccc4c3)c2)cc1 ; c1ccc(-c2cccc(N(c3ccc4c(c3)oc3ccccc34)c3ccc4oc5ccccc5c4c3-c3ccc4ccccc4c3)c2)cc1]

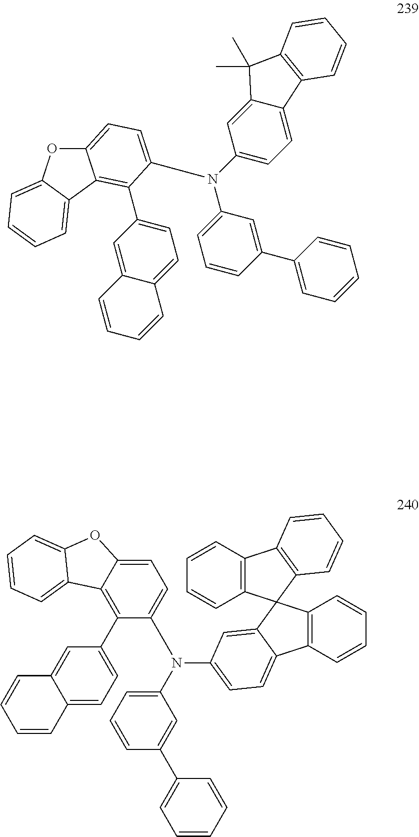 [SMILES: CC1(C)c2ccccc2-c2ccc(N(c3cccc(-c4ccccc4)c3)c3ccc4oc5ccccc5c4c3-c3ccc4ccccc4c3)cc21 ; c1ccc(-c2cccc(N(c3ccc4c(c3)C3(c5ccccc5-c5ccccc53)c3ccccc3-4)c3ccc4oc5ccccc5c4c3-c3ccc4ccccc4c3)c2)cc1]

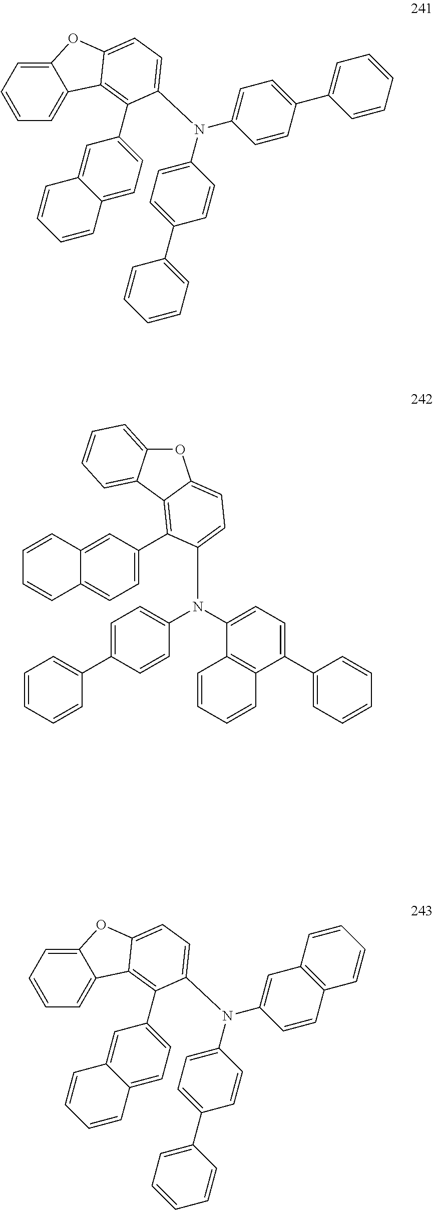 [SMILES: c1ccc(-c2ccc(N(c3ccc(-c4ccccc4)cc3)c3ccc4oc5ccccc5c4c3-c3ccc4ccccc4c3)cc2)cc1 ; c1ccc(-c2ccc(N(c3ccc4oc5ccccc5c4c3-c3ccc4ccccc4c3)c3ccc(-c4ccccc4)c4ccccc34)cc2)cc1 ; c1ccc(-c2ccc(N(c3ccc4ccccc4c3)c3ccc4oc5ccccc5c4c3-c3ccc4ccccc4c3)cc2)cc1]

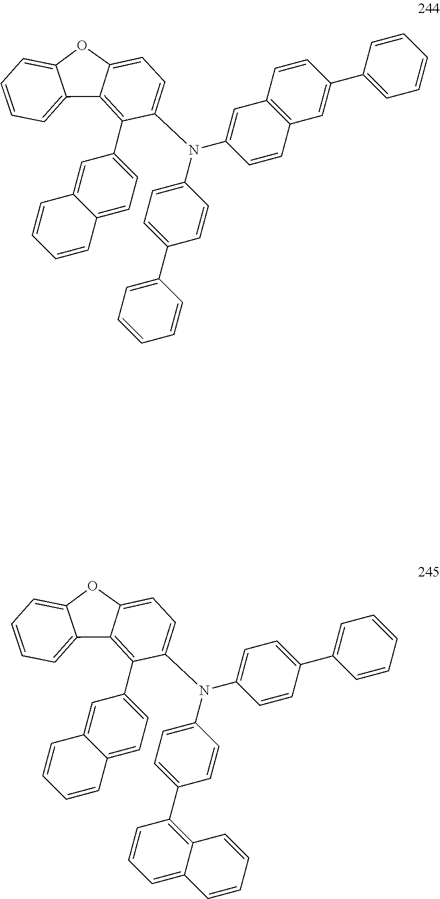 [SMILES: c1ccc(-c2ccc(N(c3ccc4cc(-c5ccccc5)ccc4c3)c3ccc4oc5ccccc5c4c3-c3ccc4ccccc4c3)cc2)cc1 ; c1ccc(-c2ccc(N(c3ccc(-c4cccc5ccccc45)cc3)c3ccc4oc5ccccc5c4c3-c3ccc4ccccc4c3)cc2)cc1]

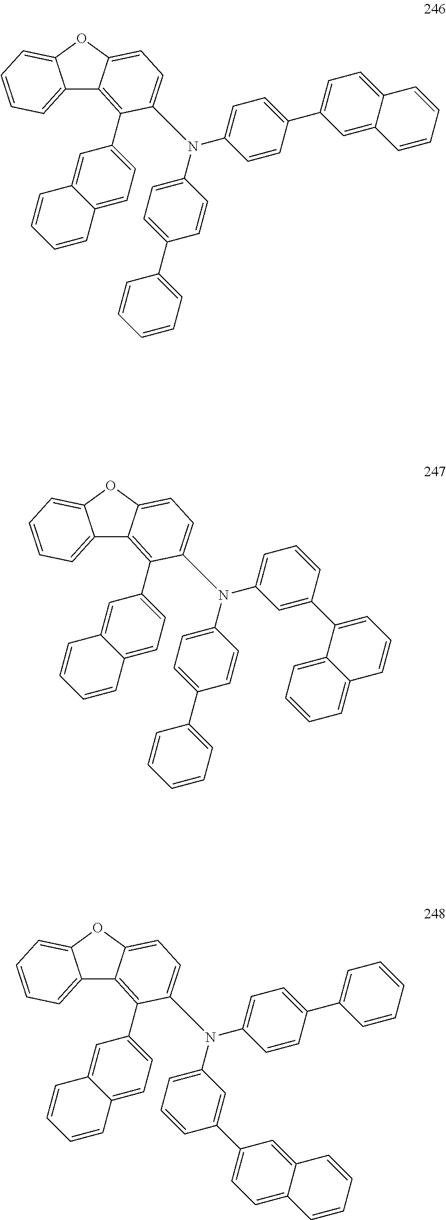 [SMILES: c1ccc(-c2ccc(N(c3ccc(-c4ccc5ccccc5c4)cc3)c3ccc4oc5ccccc5c4c3-c3ccc4ccccc4c3)cc2)cc1 ; c1ccc(-c2ccc(N(c3cccc(-c4cccc5ccccc45)c3)c3ccc4oc5ccccc5c4c3-c3ccc4ccccc4c3)cc2)cc1 ; c1ccc(-c2ccc(N(c3cccc(-c4ccc5ccccc5c4)c3)c3ccc4oc5ccccc5c4c3-c3ccc4ccccc4c3)cc2)cc1]

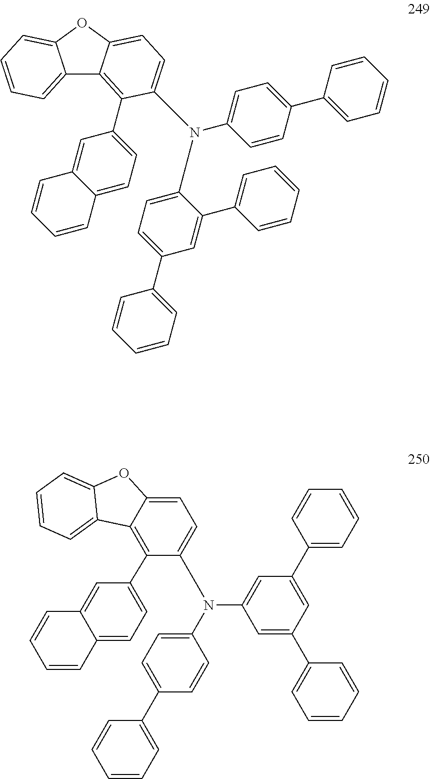 [SMILES: c1ccc(-c2ccc(N(c3ccc(-c4ccccc4)cc3-c3ccccc3)c3ccc4oc5ccccc5c4c3-c3ccc4ccccc4c3)cc2)cc1 ; c1ccc(-c2ccc(N(c3cc(-c4ccccc4)cc(-c4ccccc4)c3)c3ccc4oc5ccccc5c4c3-c3ccc4ccccc4c3)cc2)cc1]

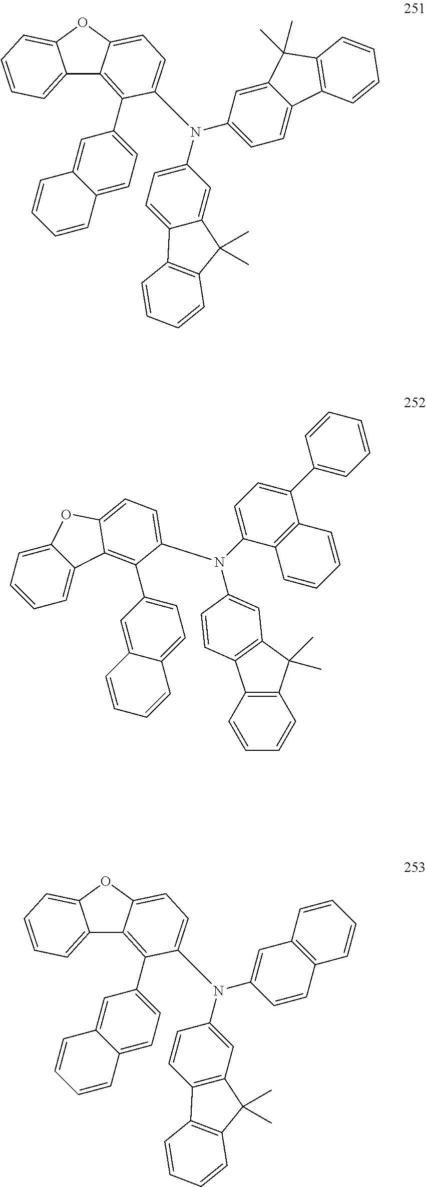 [SMILES: CC1(C)c2ccccc2-c2ccc(N(c3ccc4c(c3)C(C)(C)c3ccccc3-4)c3ccc4oc5ccccc5c4c3-c3ccc4ccccc4c3)cc21 ; CC1(C)c2ccccc2-c2ccc(N(c3ccc4oc5ccccc5c4c3-c3ccc4ccccc4c3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc4ccccc4c3)cc21]

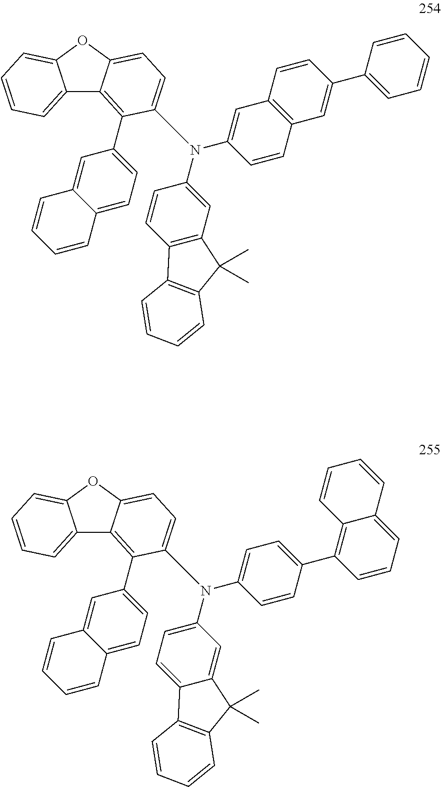 [SMILES: CC1(C)c2ccccc2-c2ccc(N(c3ccc4cc(-c5ccccc5)ccc4c3)c3ccc4oc5ccccc5c4c3-c3ccc4ccccc4c3)cc21 ; CC1(C)c2ccccc2-c2ccc(N(c3ccc(-c4cccc5ccccc45)cc3)c3ccc4oc5ccccc5c4c3-c3ccc4ccccc4c3)cc21]

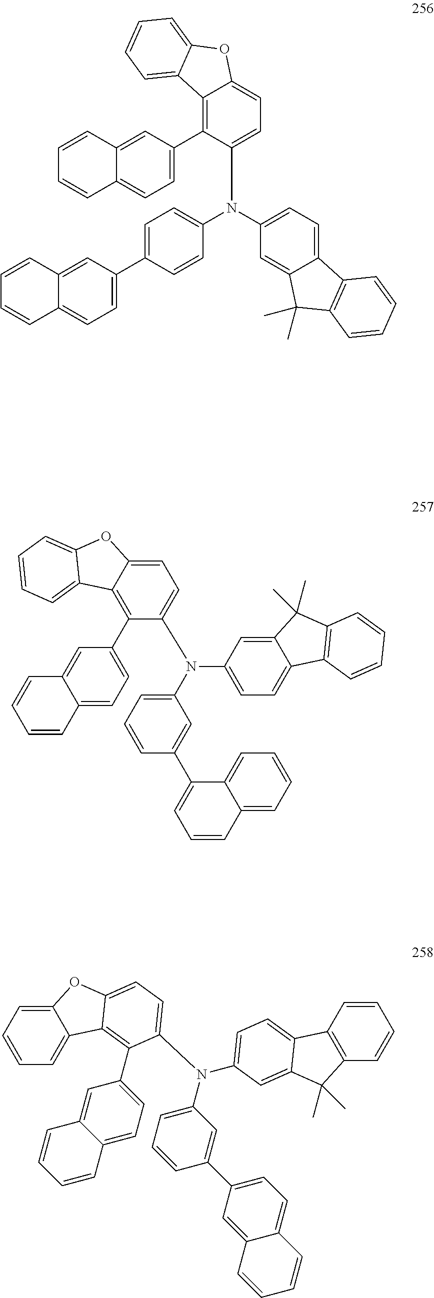 [SMILES: CC1(C)c2ccccc2-c2ccc(N(c3ccc(-c4ccc5ccccc5c4)cc3)c3ccc4oc5ccccc5c4c3-c3ccc4ccccc4c3)cc21 ; CC1(C)c2ccccc2-c2ccc(N(c3cccc(-c4cccc5ccccc45)c3)c3ccc4oc5ccccc5c4c3-c3ccc4ccccc4c3)cc21 ; CC1(C)c2ccccc2-c2ccc(N(c3cccc(-c4ccc5ccccc5c4)c3)c3ccc4oc5ccccc5c4c3-c3ccc4ccccc4c3)cc21]

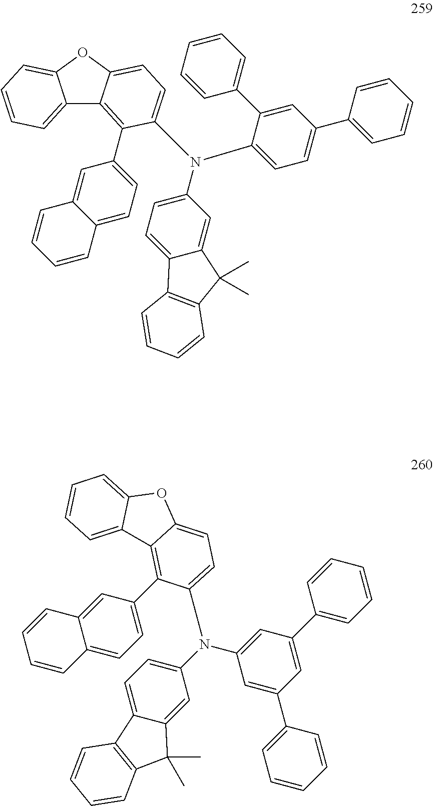 [SMILES: CC1(C)c2ccccc2-c2ccc(N(c3ccc(-c4ccccc4)cc3-c3ccccc3)c3ccc4oc5ccccc5c4c3-c3ccc4ccccc4c3)cc21 ; CC1(C)c2ccccc2-c2ccc(N(c3cc(-c4ccccc4)cc(-c4ccccc4)c3)c3ccc4oc5ccccc5c4c3-c3ccc4ccccc4c3)cc21]

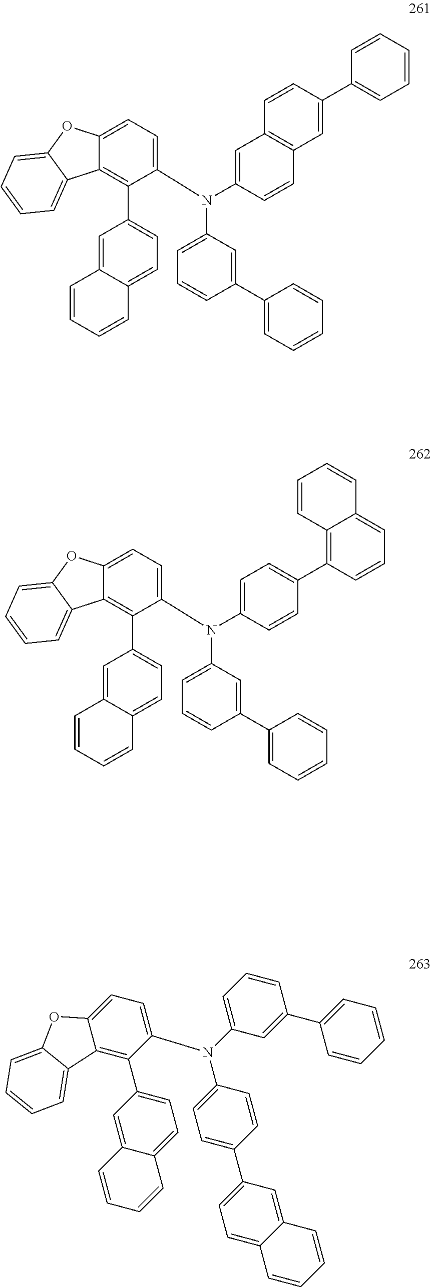 [SMILES: c1ccc(-c2cccc(N(c3ccc4cc(-c5ccccc5)ccc4c3)c3ccc4oc5ccccc5c4c3-c3ccc4ccccc4c3)c2)cc1 ; c1ccc(-c2cccc(N(c3ccc(-c4cccc5ccccc45)cc3)c3ccc4oc5ccccc5c4c3-c3ccc4ccccc4c3)c2)cc1 ; c1ccc(-c2cccc(N(c3ccc(-c4ccc5ccccc5c4)cc3)c3ccc4oc5ccccc5c4c3-c3ccc4ccccc4c3)c2)cc1]

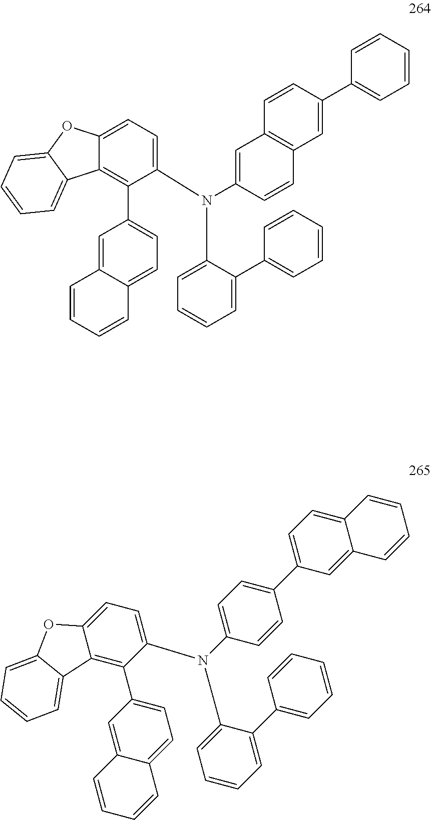 [SMILES: c1ccc(-c2ccc3cc(N(c4ccccc4-c4ccccc4)c4ccc5oc6ccccc6c5c4-c4ccc5ccccc5c4)ccc3c2)cc1 ; c1ccc(-c2ccccc2N(c2ccc(-c3ccc4ccccc4c3)cc2)c2ccc3oc4ccccc4c3c2-c2ccc3ccccc3c2)cc1]

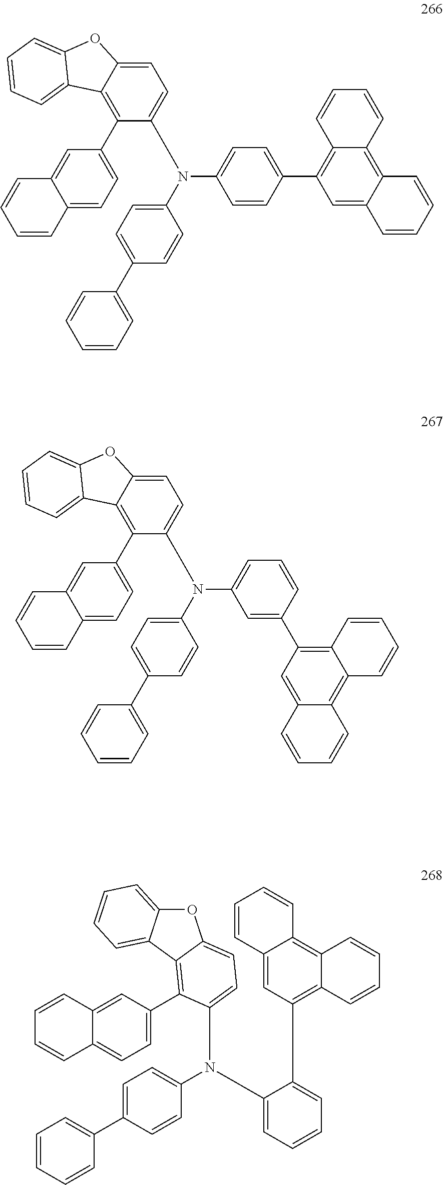 [SMILES: c1ccc(-c2ccc(N(c3ccc(-c4cc5ccccc5c5ccccc45)cc3)c3ccc4oc5ccccc5c4c3-c3ccc4ccccc4c3)cc2)cc1 ; c1ccc(-c2ccc(N(c3cccc(-c4cc5ccccc5c5ccccc45)c3)c3ccc4oc5ccccc5c4c3-c3ccc4ccccc4c3)cc2)cc1 ; c1ccc(-c2ccc(N(c3ccccc3-c3cc4ccccc4c4ccccc34)c3ccc4oc5ccccc5c4c3-c3ccc4ccccc4c3)cc2)cc1]

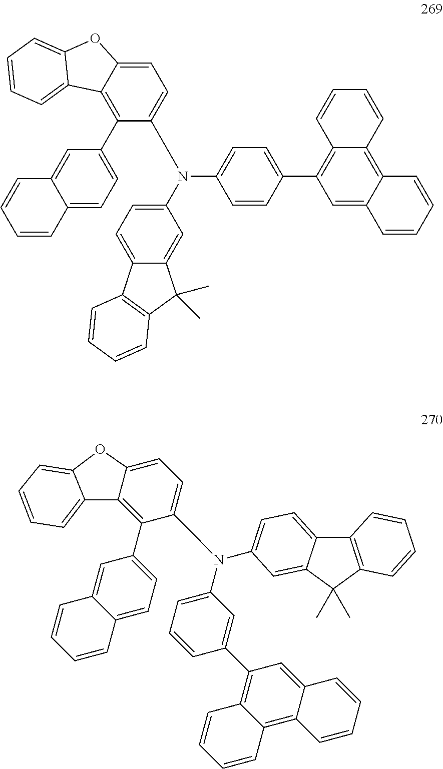 [SMILES: CC1(C)c2ccccc2-c2ccc(N(c3ccc(-c4cc5ccccc5c5ccccc45)cc3)c3ccc4oc5ccccc5c4c3-c3ccc4ccccc4c3)cc21 ; CC1(C)c2ccccc2-c2ccc(N(c3cccc(-c4cc5ccccc5c5ccccc45)c3)c3ccc4oc5ccccc5c4c3-c3ccc4ccccc4c3)cc21]

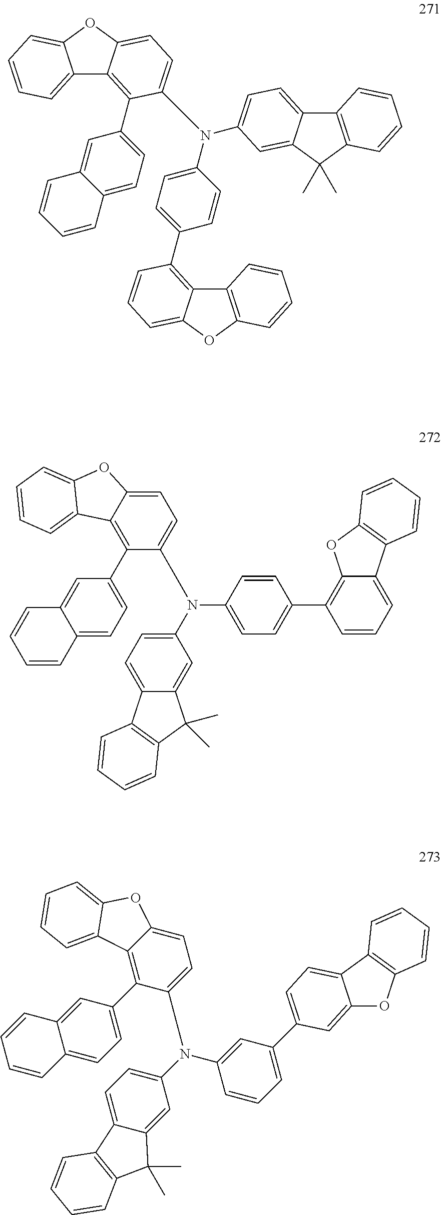 [SMILES: CC1(C)c2ccccc2-c2ccc(N(c3ccc(-c4cccc5oc6ccccc6c45)cc3)c3ccc4oc5ccccc5c4c3-c3ccc4ccccc4c3)cc21 ; CC1(C)c2ccccc2-c2ccc(N(c3ccc(-c4cccc5c4oc4ccccc45)cc3)c3ccc4oc5ccccc5c4c3-c3ccc4ccccc4c3)cc21 ; CC1(C)c2ccccc2-c2ccc(N(c3cccc(-c4ccc5c(c4)oc4ccccc45)c3)c3ccc4oc5ccccc5c4c3-c3ccc4ccccc4c3)cc21]

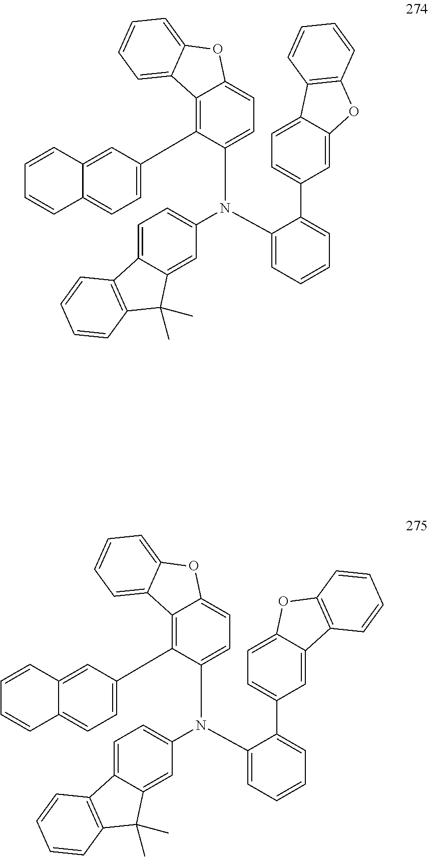 [SMILES: CC1(C)c2ccccc2-c2ccc(N(c3ccccc3-c3ccc4c(c3)oc3ccccc34)c3ccc4oc5ccccc5c4c3-c3ccc4ccccc4c3)cc21 ; CC1(C)c2ccccc2-c2ccc(N(c3ccccc3-c3ccc4oc5ccccc5c4c3)c3ccc4oc5ccccc5c4c3-c3ccc4ccccc4c3)cc21]

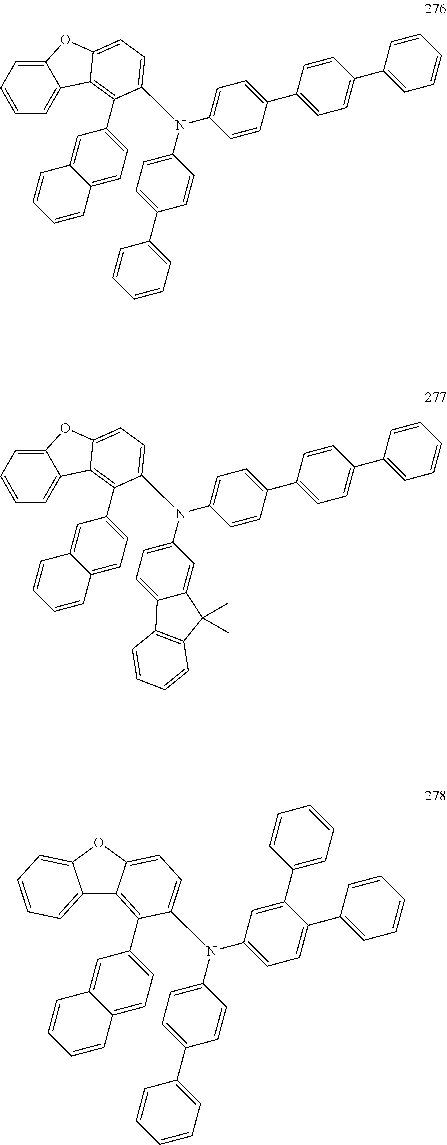 [SMILES: c1ccc(-c2ccc(-c3ccc(N(c4ccc(-c5ccccc5)cc4)c4ccc5oc6ccccc6c5c4-c4ccc5ccccc5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4ccccc4c3)cc21 ; c1ccc(-c2ccc(N(c3ccc(-c4ccccc4)c(-c4ccccc4)c3)c3ccc4oc5ccccc5c4c3-c3ccc4ccccc4c3)cc2)cc1]

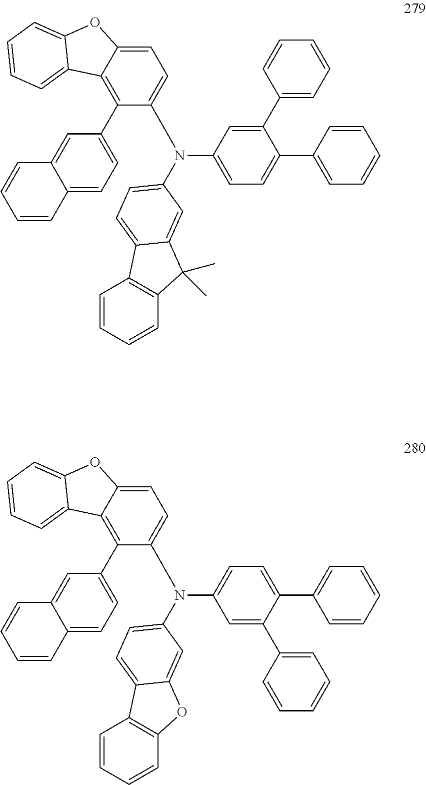 [SMILES: CC1(C)c2ccccc2-c2ccc(N(c3ccc(-c4ccccc4)c(-c4ccccc4)c3)c3ccc4oc5ccccc5c4c3-c3ccc4ccccc4c3)cc21 ; c1ccc(-c2ccc(N(c3ccc4c(c3)oc3ccccc34)c3ccc4oc5ccccc5c4c3-c3ccc4ccccc4c3)cc2-c2ccccc2)cc1]

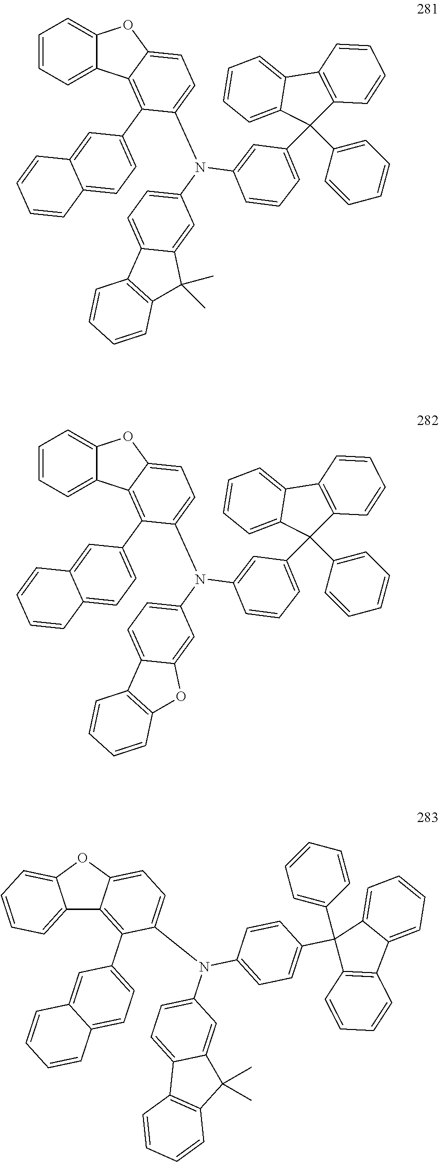 [SMILES: CC1(C)c2ccccc2-c2ccc(N(c3cccc(C4(c5ccccc5)c5ccccc5-c5ccccc54)c3)c3ccc4oc5ccccc5c4c3-c3ccc4ccccc4c3)cc21 ; c1ccc(C2(c3cccc(N(c4ccc5c(c4)oc4ccccc45)c4ccc5oc6ccccc6c5c4-c4ccc5ccccc5c4)c3)c3ccccc3-c3ccccc32)cc1 ; CC1(C)c2ccccc2-c2ccc(N(c3ccc(C4(c5ccccc5)c5ccccc5-c5ccccc54)cc3)c3ccc4oc5ccccc5c4c3-c3ccc4ccccc4c3)cc21]

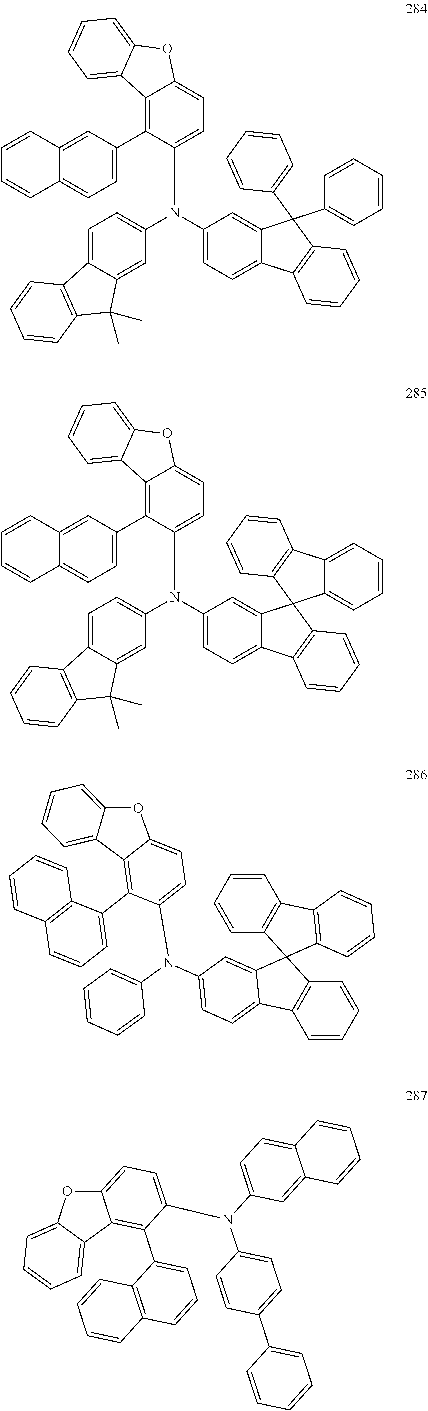 [SMILES: CC1(C)c2ccccc2-c2ccc(N(c3ccc4c(c3)C(c3ccccc3)(c3ccccc3)c3ccccc3-4)c3ccc4oc5ccccc5c4c3-c3ccc4ccccc4c3)cc21 ; CC1(C)c2ccccc2-c2ccc(N(c3ccc4c(c3)C3(c5ccccc5-c5ccccc53)c3ccccc3-4)c3ccc4oc5ccccc5c4c3-c3ccc4ccccc4c3)cc21 ; c1ccc(N(c2ccc3c(c2)C2(c4ccccc4-c4ccccc42)c2ccccc2-3)c2ccc3oc4ccccc4c3c2-c2cccc3ccccc23)cc1 ; c1ccc(-c2ccc(N(c3ccc4ccccc4c3)c3ccc4oc5ccccc5c4c3-c3cccc4ccccc34)cc2)cc1]

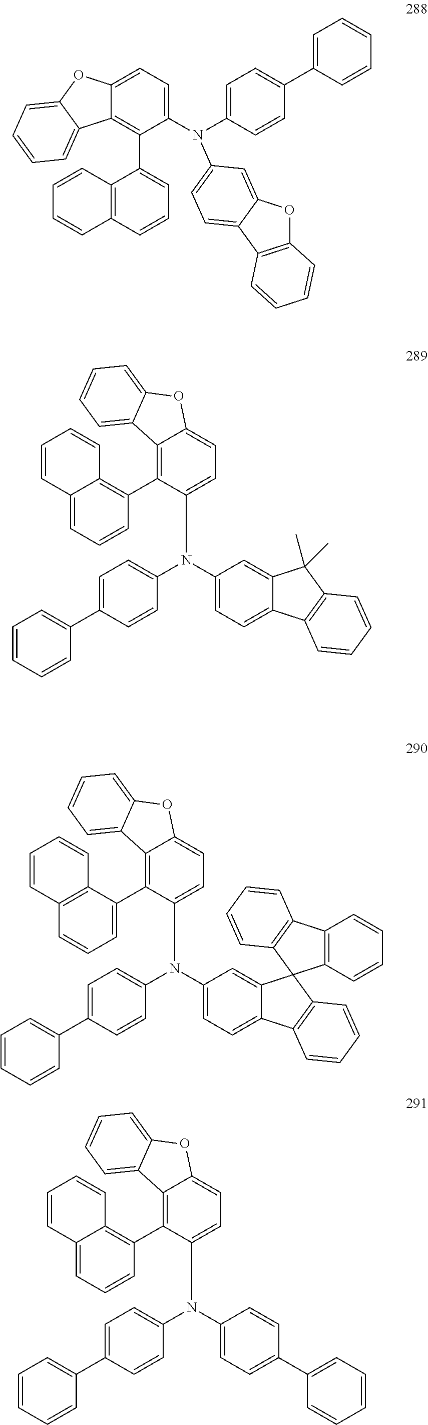 [SMILES: c1ccc(-c2ccc(N(c3ccc4c(c3)oc3ccccc34)c3ccc4oc5ccccc5c4c3-c3cccc4ccccc34)cc2)cc1 ; CC1(C)c2ccccc2-c2ccc(N(c3ccc(-c4ccccc4)cc3)c3ccc4oc5ccccc5c4c3-c3cccc4ccccc34)cc21 ; c1ccc(-c2ccc(N(c3ccc4c(c3)C3(c5ccccc5-c5ccccc53)c3ccccc3-4)c3ccc4oc5ccccc5c4c3-c3cccc4ccccc34)cc2)cc1 ; c1ccc(-c2ccc(N(c3ccc(-c4ccccc4)cc3)c3ccc4oc5ccccc5c4c3-c3cccc4ccccc34)cc2)cc1]

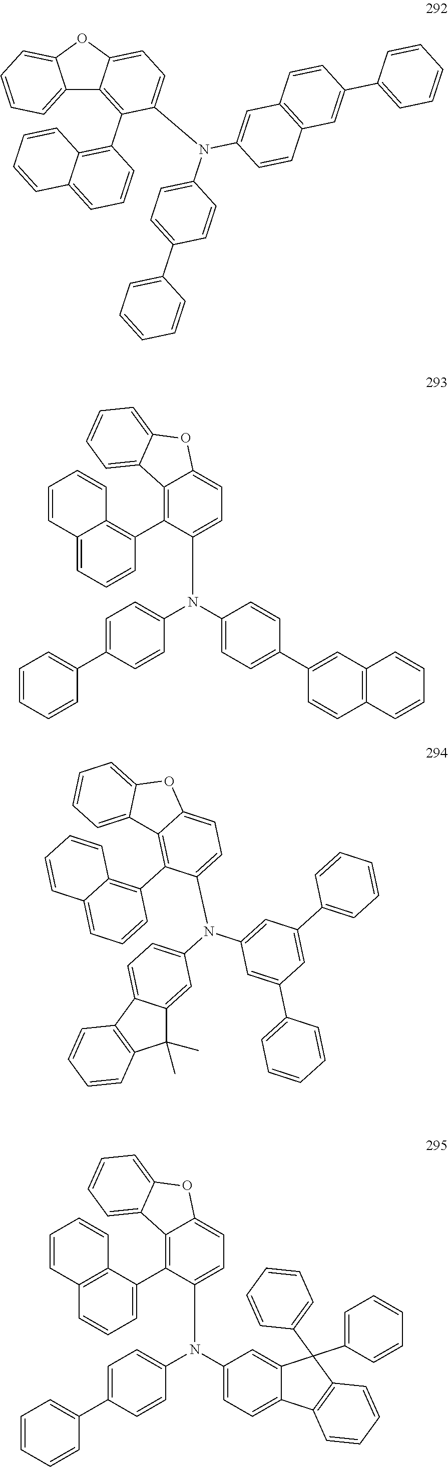 [SMILES: c1ccc(-c2ccc(N(c3ccc4cc(-c5ccccc5)ccc4c3)c3ccc4oc5ccccc5c4c3-c3cccc4ccccc34)cc2)cc1 ; c1ccc(-c2ccc(N(c3ccc(-c4ccc5ccccc5c4)cc3)c3ccc4oc5ccccc5c4c3-c3cccc4ccccc34)cc2)cc1 ; CC1(C)c2ccccc2-c2ccc(N(c3cc(-c4ccccc4)cc(-c4ccccc4)c3)c3ccc4oc5ccccc5c4c3-c3cccc4ccccc34)cc21 ; c1ccc(-c2ccc(N(c3ccc4c(c3)C(c3ccccc3)(c3ccccc3)c3ccccc3-4)c3ccc4oc5ccccc5c4c3-c3cccc4ccccc34)cc2)cc1]

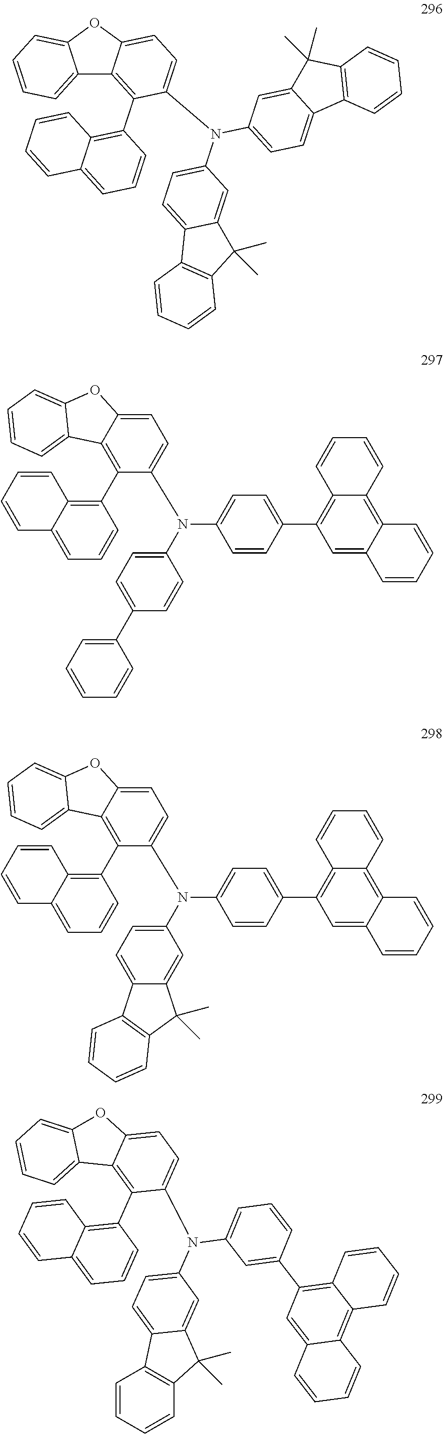

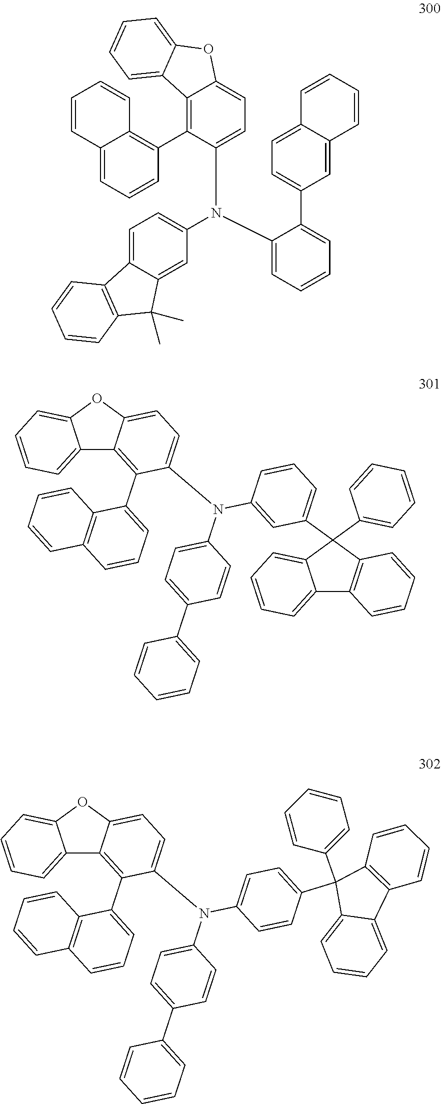 [SMILES: CC1(C)c2ccccc2-c2ccc(N(c3ccccc3-c3ccc4ccccc4c3)c3ccc4oc5ccccc5c4c3-c3cccc4ccccc34)cc21 ; c1ccc(-c2ccc(N(c3cccc(C4(c5ccccc5)c5ccccc5-c5ccccc54)c3)c3ccc4oc5ccccc5c4c3-c3cccc4ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cccc4ccccc34)cc2)cc1]

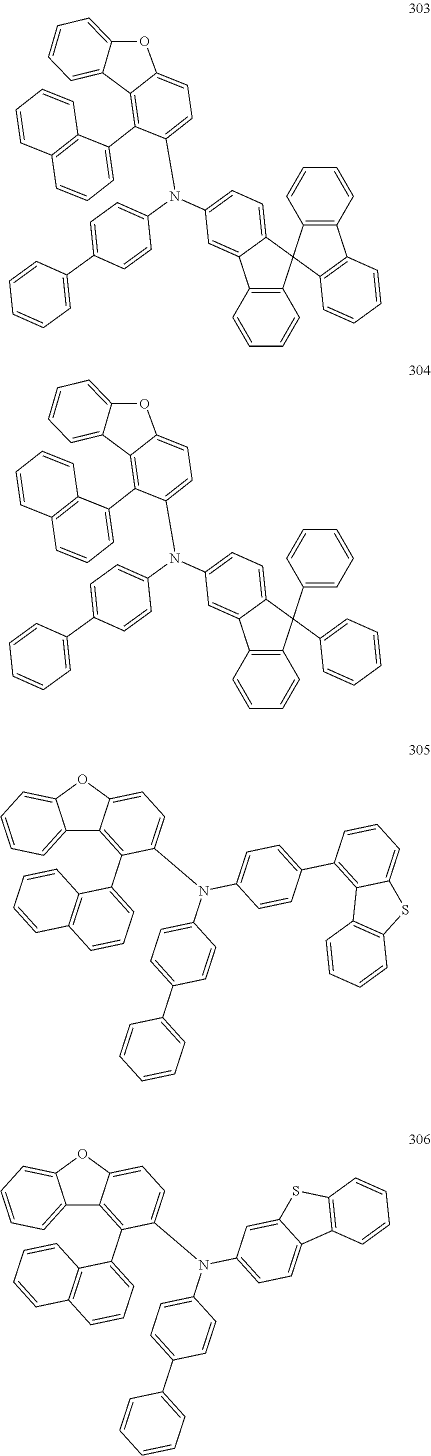 [SMILES: c1ccc(-c2ccc(N(c3ccc4c(c3)-c3ccccc3C43c4ccccc4-c4ccccc43)c3ccc4oc5ccccc5c4c3-c3cccc4ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3cccc4ccccc34)cc2)cc1 ; c1ccc(-c2ccc(N(c3ccc(-c4cccc5sc6ccccc6c45)cc3)c3ccc4oc5ccccc5c4c3-c3cccc4ccccc34)cc2)cc1 ; c1ccc(-c2ccc(N(c3ccc4c(c3)sc3ccccc34)c3ccc4oc5ccccc5c4c3-c3cccc4ccccc34)cc2)cc1]

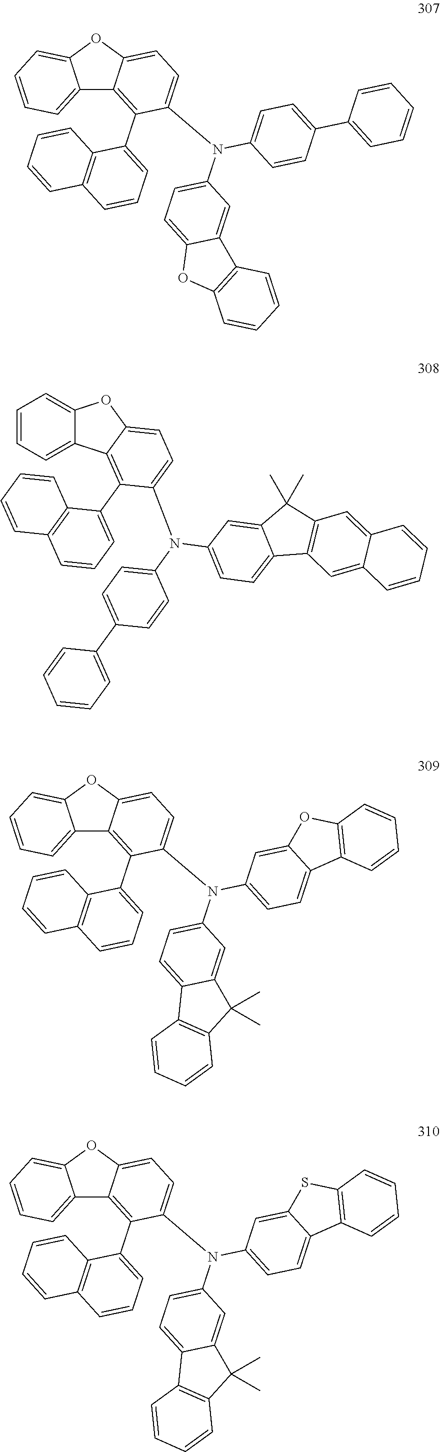 [SMILES: c1ccc(-c2ccc(N(c3ccc4oc5ccccc5c4c3)c3ccc4oc5ccccc5c4c3-c3cccc4ccccc34)cc2)cc1 ; CC1(C)c2cc(N(c3ccc(-c4ccccc4)cc3)c3ccc4oc5ccccc5c4c3-c3cccc4ccccc34)ccc2-c2cc3ccccc3cc21 ; CC1(C)c2ccccc2-c2ccc(N(c3ccc4c(c3)oc3ccccc34)c3ccc4oc5ccccc5c4c3-c3cccc4ccccc34)cc21 ; CC1(C)c2ccccc2-c2ccc(N(c3ccc4c(c3)sc3ccccc34)c3ccc4oc5ccccc5c4c3-c3cccc4ccccc34)cc21]

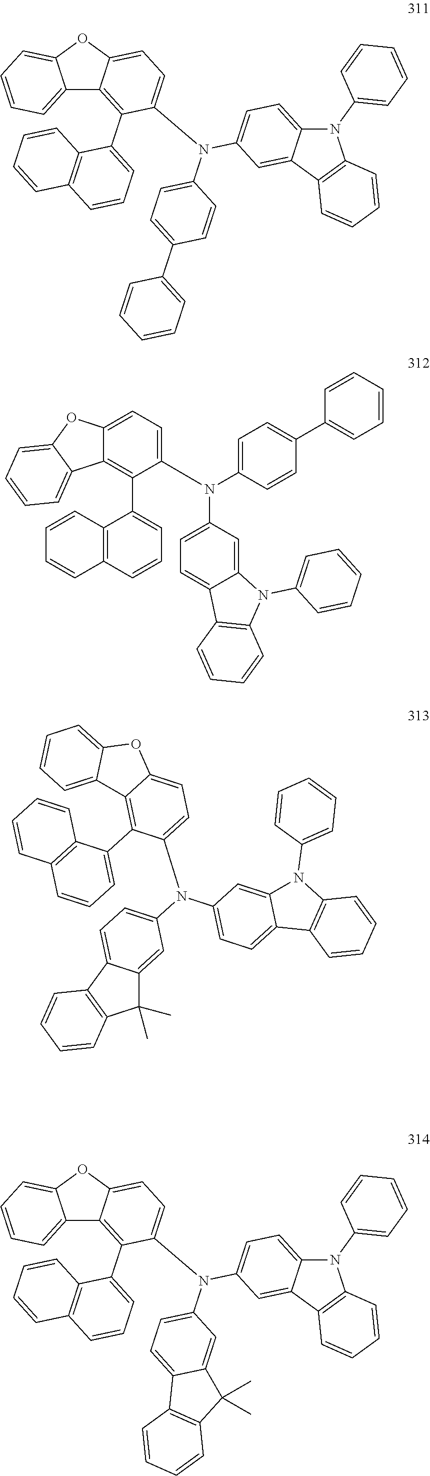 [SMILES: c1ccc(-c2ccc(N(c3ccc4c(c3)c3ccccc3n4-c3ccccc3)c3ccc4oc5ccccc5c4c3-c3cccc4ccccc34)cc2)cc1 ; c1ccc(-c2ccc(N(c3ccc4c5ccccc5n(-c5ccccc5)c4c3)c3ccc4oc5ccccc5c4c3-c3cccc4ccccc34)cc2)cc1 ; CC1(C)c2ccccc2-c2ccc(N(c3ccc4c5ccccc5n(-c5ccccc5)c4c3)c3ccc4oc5ccccc5c4c3-c3cccc4ccccc34)cc21 ; CC1(C)c2ccccc2-c2ccc(N(c3ccc4c(c3)c3ccccc3n4-c3ccccc3)c3ccc4oc5ccccc5c4c3-c3cccc4ccccc34)cc21]

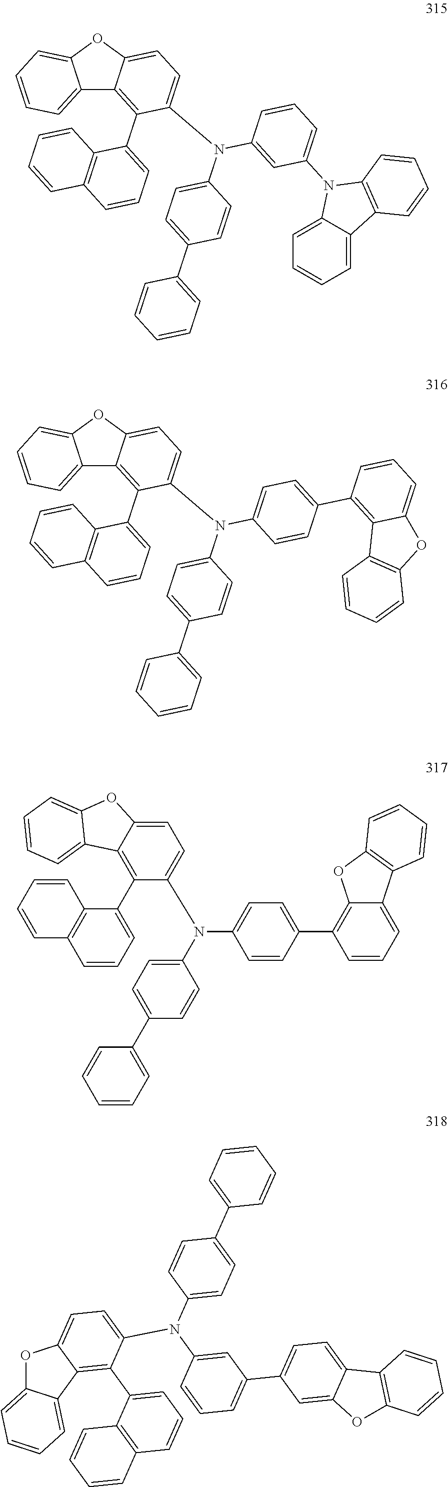 [SMILES: c1ccc(-c2ccc(N(c3cccc(-n4c5ccccc5c5ccccc54)c3)c3ccc4oc5ccccc5c4c3-c3cccc4ccccc34)cc2)cc1 ; c1ccc(-c2ccc(N(c3ccc(-c4cccc5oc6ccccc6c45)cc3)c3ccc4oc5ccccc5c4c3-c3cccc4ccccc34)cc2)cc1 ; c1ccc(-c2ccc(N(c3ccc(-c4cccc5c4oc4ccccc45)cc3)c3ccc4oc5ccccc5c4c3-c3cccc4ccccc34)cc2)cc1 ; c1ccc(-c2ccc(N(c3cccc(-c4ccc5c(c4)oc4ccccc45)c3)c3ccc4oc5ccccc5c4c3-c3cccc4ccccc34)cc2)cc1]

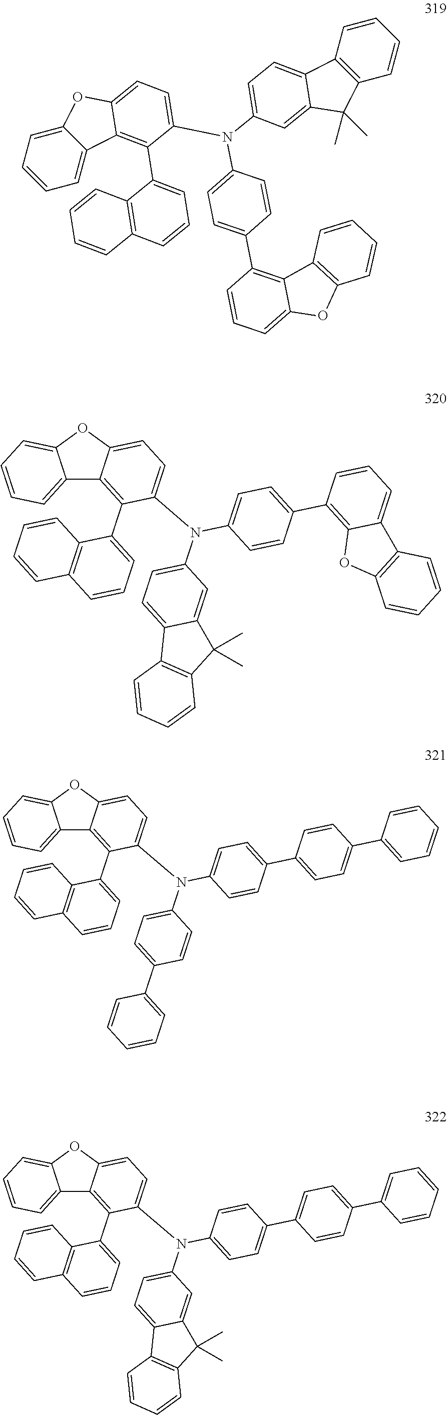 [SMILES: CC1(C)c2ccccc2-c2ccc(N(c3ccc(-c4cccc5oc6ccccc6c45)cc3)c3ccc4oc5ccccc5c4c3-c3cccc4ccccc34)cc21 ; CC1(C)c2ccccc2-c2ccc(N(c3ccc(-c4cccc5c4oc4ccccc45)cc3)c3ccc4oc5ccccc5c4c3-c3cccc4ccccc34)cc21 ; c1ccc(-c2ccc(-c3ccc(N(c4ccc(-c5ccccc5)cc4)c4ccc5oc6ccccc6c5c4-c4cccc5ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc4ccccc34)cc21]

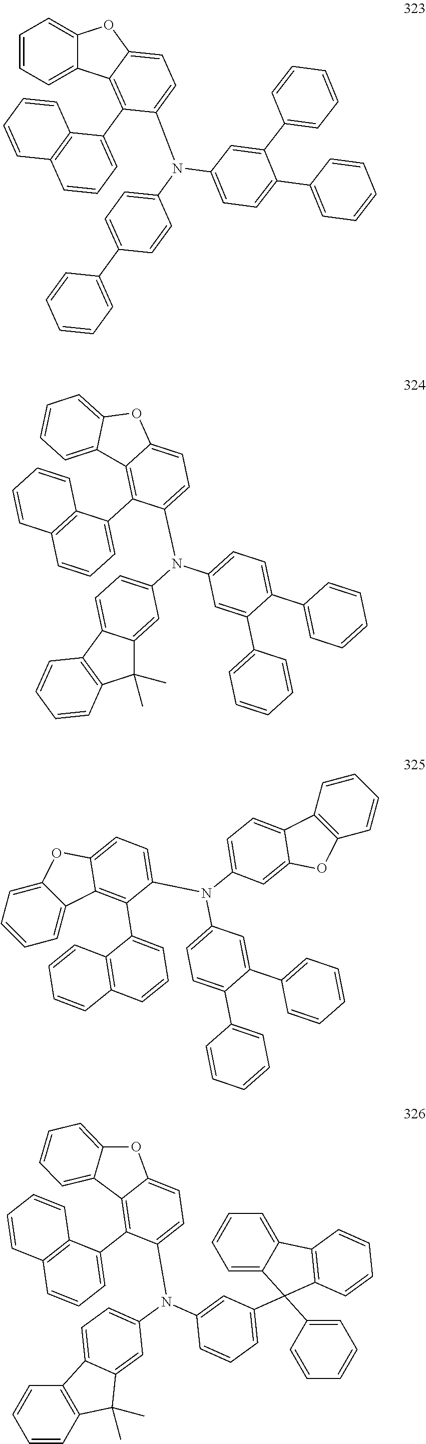 [SMILES: c1ccc(-c2ccc(N(c3ccc(-c4ccccc4)c(-c4ccccc4)c3)c3ccc4oc5ccccc5c4c3-c3cccc4ccccc34)cc2)cc1 ; CC1(C)c2ccccc2-c2ccc(N(c3ccc(-c4ccccc4)c(-c4ccccc4)c3)c3ccc4oc5ccccc5c4c3-c3cccc4ccccc34)cc21 ; c1ccc(-c2ccc(N(c3ccc4c(c3)oc3ccccc34)c3ccc4oc5ccccc5c4c3-c3cccc4ccccc34)cc2-c2ccccc2)cc1 ; CC1(C)c2ccccc2-c2ccc(N(c3cccc(C4(c5ccccc5)c5ccccc5-c5ccccc54)c3)c3ccc4oc5ccccc5c4c3-c3cccc4ccccc34)cc21]

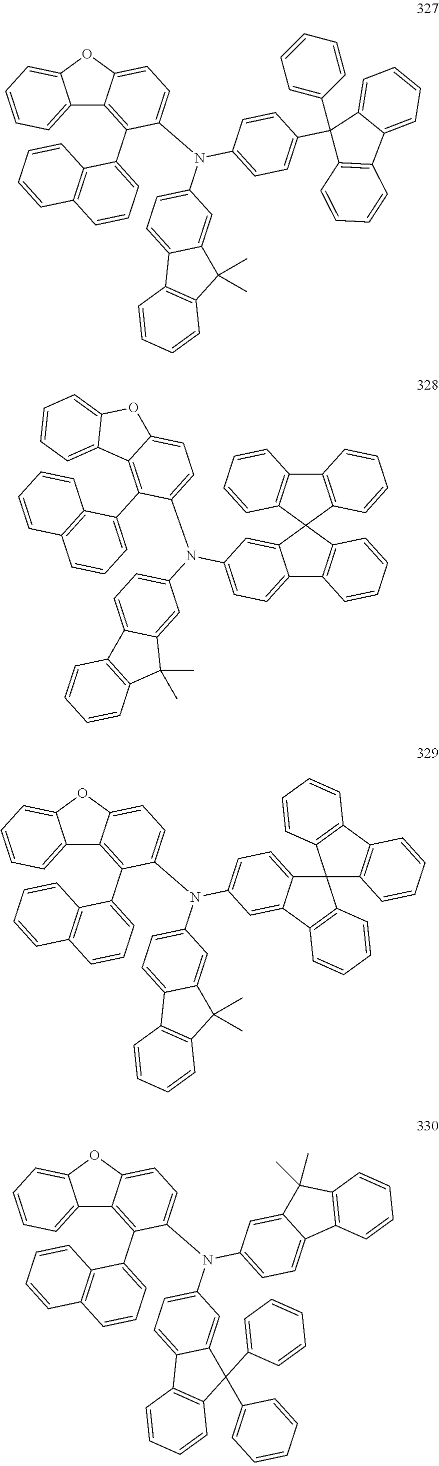

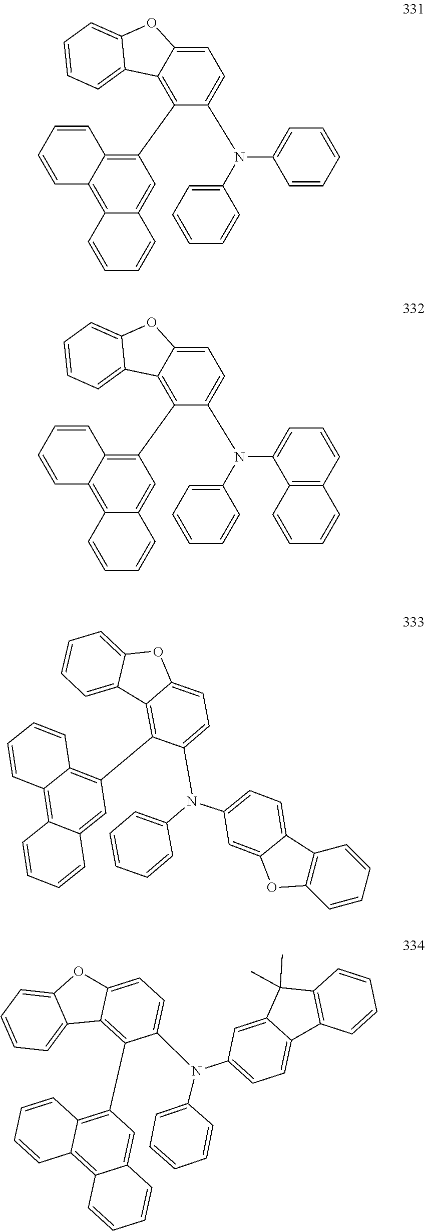 [SMILES: c1ccc(N(c2ccccc2)c2ccc3oc4ccccc4c3c2-c2cc3ccccc3c3ccccc23)cc1 ; c1ccc(N(c2ccc3oc4ccccc4c3c2-c2cc3ccccc3c3ccccc23)c2cccc3ccccc23)cc1 ; c1ccc(N(c2ccc3c(c2)oc2ccccc23)c2ccc3oc4ccccc4c3c2-c2cc3ccccc3c3ccccc23)cc1 ; CC1(C)c2ccccc2-c2ccc(N(c3ccccc3)c3ccc4oc5ccccc5c4c3-c3cc4ccccc4c4ccccc34)cc21]

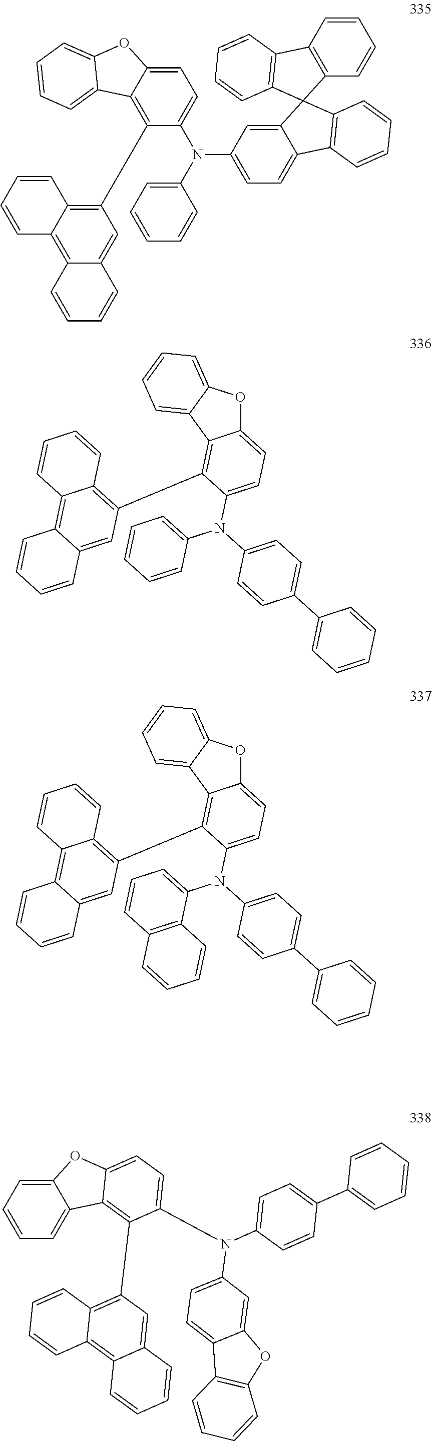 [SMILES: c1ccc(N(c2ccc3c(c2)C2(c4ccccc4-c4ccccc42)c2ccccc2-3)c2ccc3oc4ccccc4c3c2-c2cc3ccccc3c3ccccc23)cc1 ; c1ccc(-c2ccc(N(c3ccccc3)c3ccc4oc5ccccc5c4c3-c3cc4ccccc4c4ccccc34)cc2)cc1 ; c1ccc(-c2ccc(N(c3ccc4oc5ccccc5c4c3-c3cc4ccccc4c4ccccc34)c3cccc4ccccc34)cc2)cc1 ; c1ccc(-c2ccc(N(c3ccc4c(c3)oc3ccccc34)c3ccc4oc5ccccc5c4c3-c3cc4ccccc4c4ccccc34)cc2)cc1]

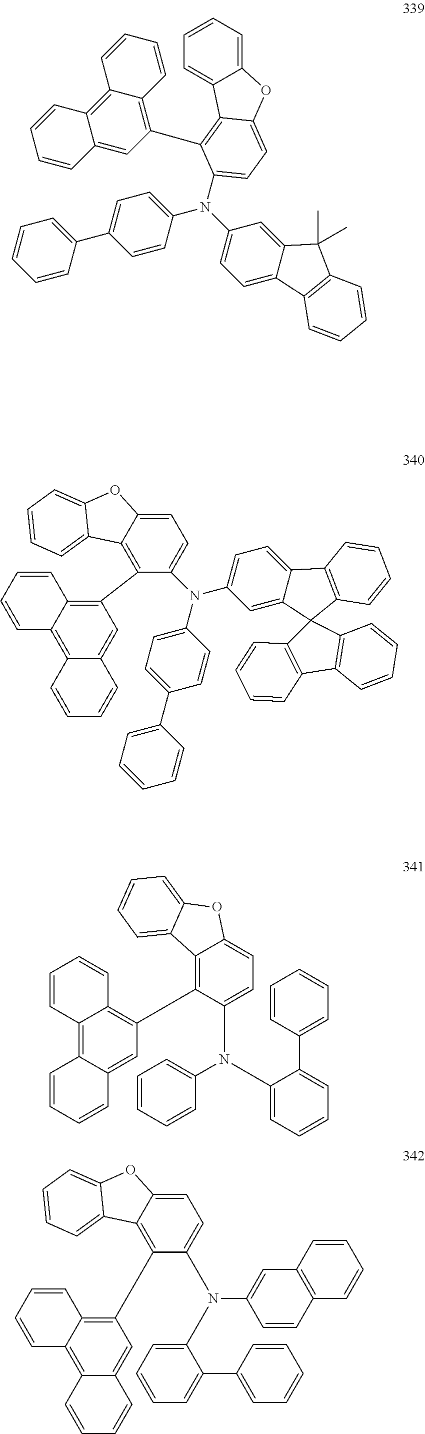 [SMILES: CC1(C)c2ccccc2-c2ccc(N(c3ccc(-c4ccccc4)cc3)c3ccc4oc5ccccc5c4c3-c3cc4ccccc4c4ccccc34)cc21 ; c1ccc(-c2ccc(N(c3ccc4c(c3)C3(c5ccccc5-c5ccccc53)c3ccccc3-4)c3ccc4oc5ccccc5c4c3-c3cc4ccccc4c4ccccc34)cc2)cc1 ; c1ccc(-c2ccccc2N(c2ccccc2)c2ccc3oc4ccccc4c3c2-c2cc3ccccc3c3ccccc23)cc1 ; c1ccc(-c2ccccc2N(c2ccc3ccccc3c2)c2ccc3oc4ccccc4c3c2-c2cc3ccccc3c3ccccc23)cc1]

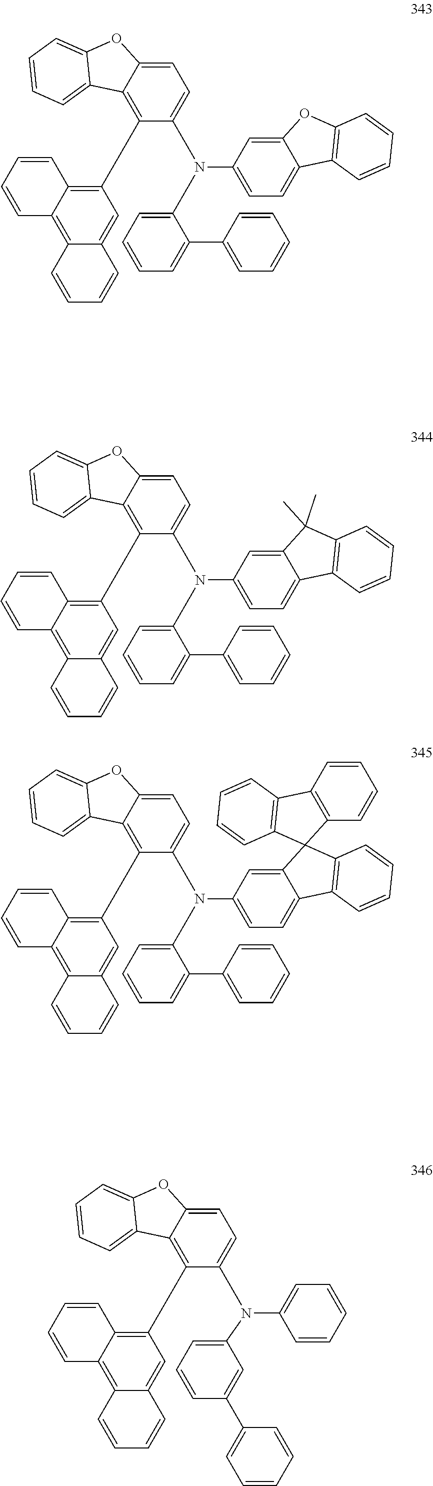 [SMILES: c1ccc(-c2ccccc2N(c2ccc3c(c2)oc2ccccc23)c2ccc3oc4ccccc4c3c2-c2cc3ccccc3c3ccccc23)cc1 ; CC1(C)c2ccccc2-c2ccc(N(c3ccccc3-c3ccccc3)c3ccc4oc5ccccc5c4c3-c3cc4ccccc4c4ccccc34)cc21 ; c1ccc(-c2ccccc2N(c2ccc3c(c2)C2(c4ccccc4-c4ccccc42)c2ccccc2-3)c2ccc3oc4ccccc4c3c2-c2cc3ccccc3c3ccccc23)cc1 ; c1ccc(-c2cccc(N(c3ccccc3)c3ccc4oc5ccccc5c4c3-c3cc4ccccc4c4ccccc34)c2)cc1]

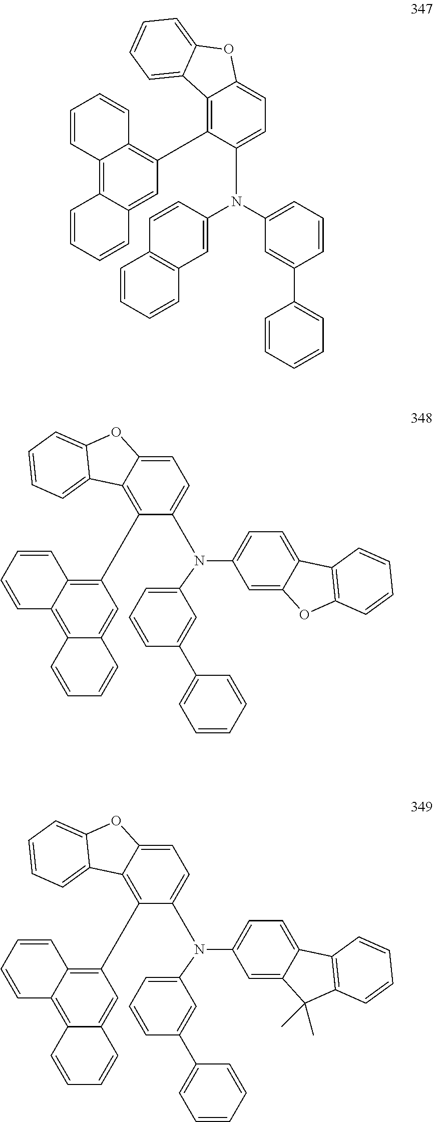 [SMILES: c1ccc(-c2cccc(N(c3ccc4ccccc4c3)c3ccc4oc5ccccc5c4c3-c3cc4ccccc4c4ccccc34)c2)cc1 ; c1ccc(-c2cccc(N(c3ccc4c(c3)oc3ccccc34)c3ccc4oc5ccccc5c4c3-c3cc4ccccc4c4ccccc34)c2)cc1 ; CC1(C)c2ccccc2-c2ccc(N(c3cccc(-c4ccccc4)c3)c3ccc4oc5ccccc5c4c3-c3cc4ccccc4c4ccccc34)cc21]

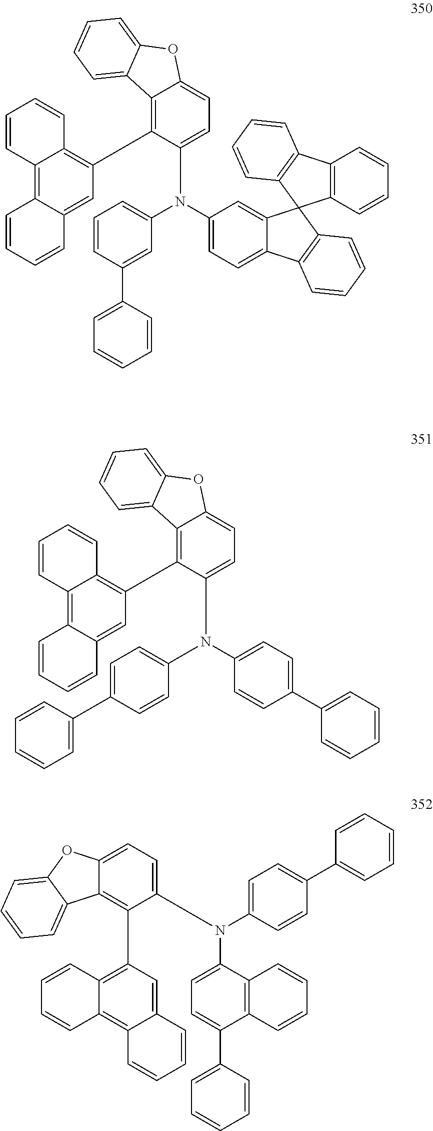 [SMILES: c1ccc(-c2cccc(N(c3ccc4c(c3)C3(c5ccccc5-c5ccccc53)c3ccccc3-4)c3ccc4oc5ccccc5c4c3-c3cc4ccccc4c4ccccc34)c2)cc1 ; c1ccc(-c2ccc(N(c3ccc(-c4ccccc4)cc3)c3ccc4oc5ccccc5c4c3-c3cc4ccccc4c4ccccc34)cc2)cc1 ; c1ccc(-c2ccc(N(c3ccc4oc5ccccc5c4c3-c3cc4ccccc4c4ccccc34)c3ccc(-c4ccccc4)c4ccccc34)cc2)cc1]

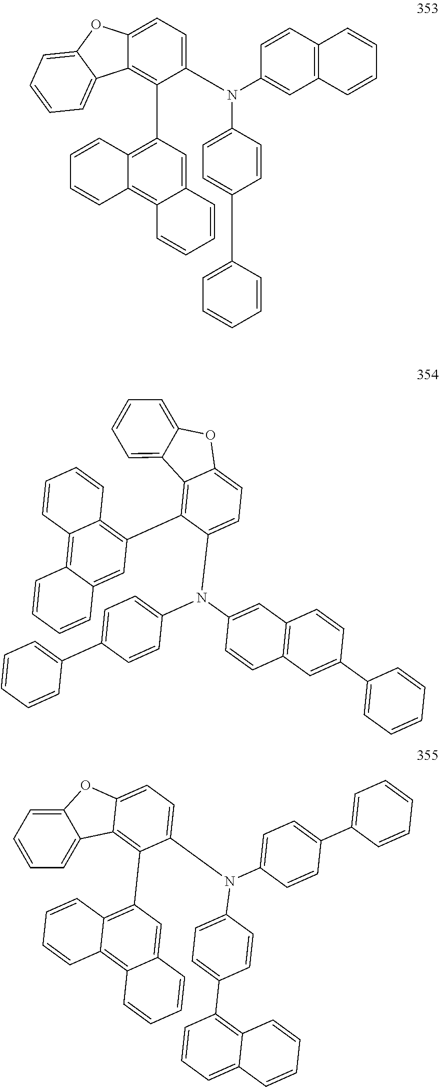

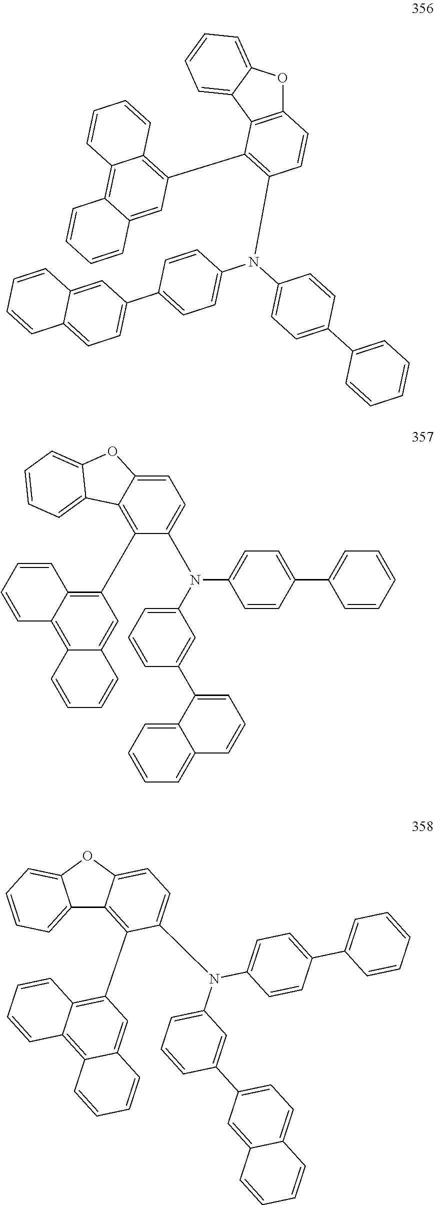 [SMILES: c1ccc(-c2ccc(N(c3ccc(-c4ccc5ccccc5c4)cc3)c3ccc4oc5ccccc5c4c3-c3cc4ccccc4c4ccccc34)cc2)cc1 ; c1ccc(-c2ccc(N(c3cccc(-c4cccc5ccccc45)c3)c3ccc4oc5ccccc5c4c3-c3cc4ccccc4c4ccccc34)cc2)cc1 ; c1ccc(-c2ccc(N(c3cccc(-c4ccc5ccccc5c4)c3)c3ccc4oc5ccccc5c4c3-c3cc4ccccc4c4ccccc34)cc2)cc1]

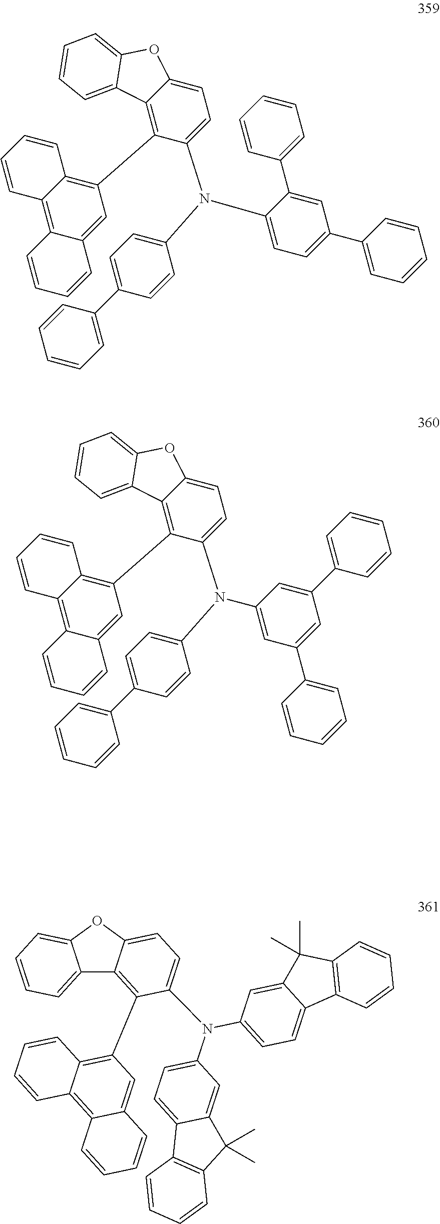 [SMILES: c1ccc(-c2ccc(N(c3ccc(-c4ccccc4)cc3-c3ccccc3)c3ccc4oc5ccccc5c4c3-c3cc4ccccc4c4ccccc34)cc2)cc1 ; c1ccc(-c2ccc(N(c3cc(-c4ccccc4)cc(-c4ccccc4)c3)c3ccc4oc5ccccc5c4c3-c3cc4ccccc4c4ccccc34)cc2)cc1 ; CC1(C)c2ccccc2-c2ccc(N(c3ccc4c(c3)C(C)(C)c3ccccc3-4)c3ccc4oc5ccccc5c4c3-c3cc4ccccc4c4ccccc34)cc21]

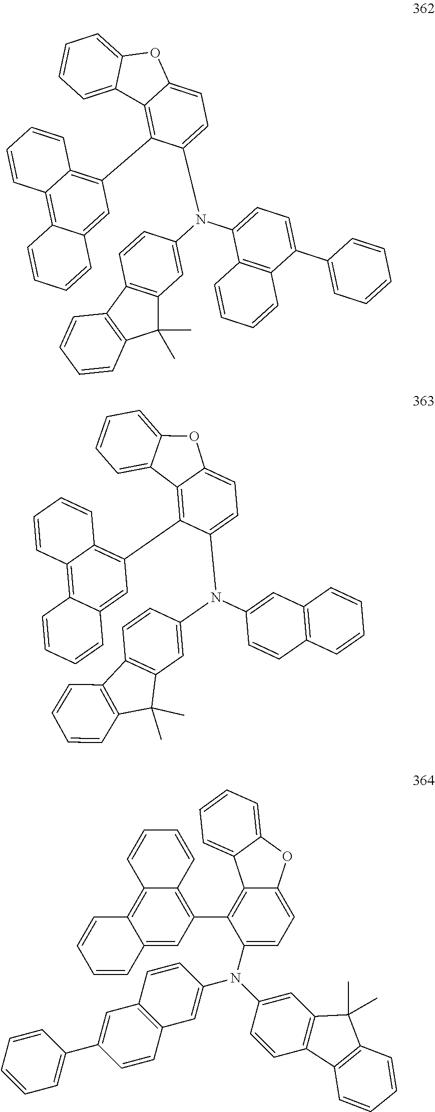

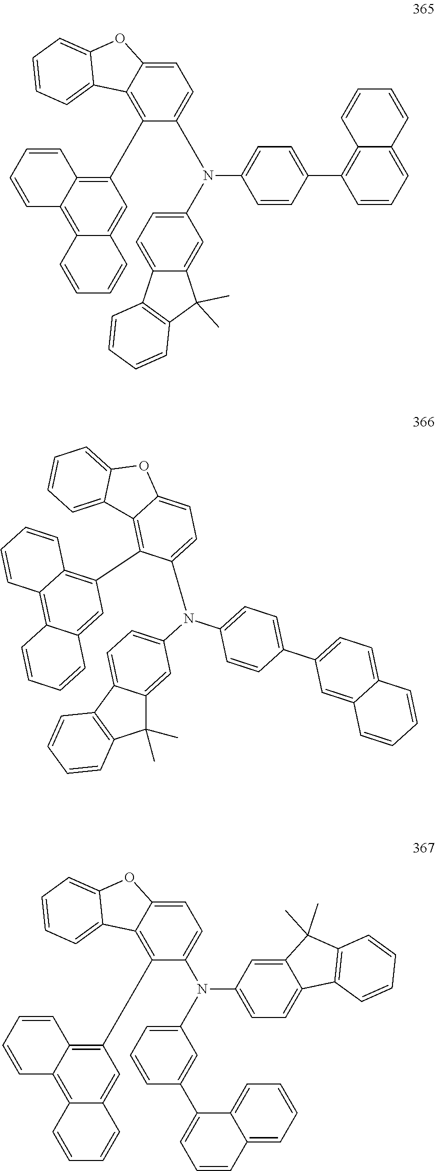 [SMILES: CC1(C)c2ccccc2-c2ccc(N(c3ccc(-c4cccc5ccccc45)cc3)c3ccc4oc5ccccc5c4c3-c3cc4ccccc4c4ccccc34)cc21 ; CC1(C)c2ccccc2-c2ccc(N(c3ccc(-c4ccc5ccccc5c4)cc3)c3ccc4oc5ccccc5c4c3-c3cc4ccccc4c4ccccc34)cc21 ; CC1(C)c2ccccc2-c2ccc(N(c3cccc(-c4cccc5ccccc45)c3)c3ccc4oc5ccccc5c4c3-c3cc4ccccc4c4ccccc34)cc21]

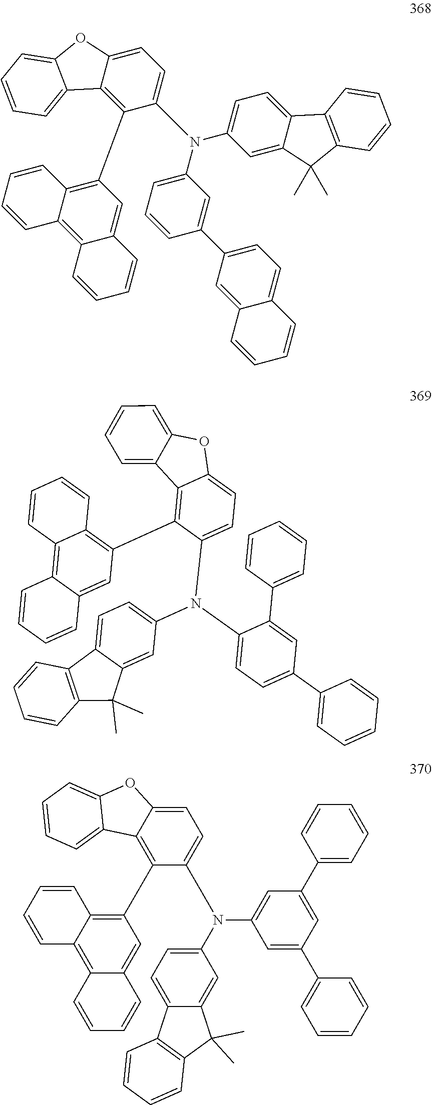 [SMILES: CC1(C)c2ccccc2-c2ccc(N(c3cccc(-c4ccc5ccccc5c4)c3)c3ccc4oc5ccccc5c4c3-c3cc4ccccc4c4ccccc34)cc21 ; CC1(C)c2ccccc2-c2ccc(N(c3ccc(-c4ccccc4)cc3-c3ccccc3)c3ccc4oc5ccccc5c4c3-c3cc4ccccc4c4ccccc34)cc21 ; CC1(C)c2ccccc2-c2ccc(N(c3cc(-c4ccccc4)cc(-c4ccccc4)c3)c3ccc4oc5ccccc5c4c3-c3cc4ccccc4c4ccccc34)cc21]

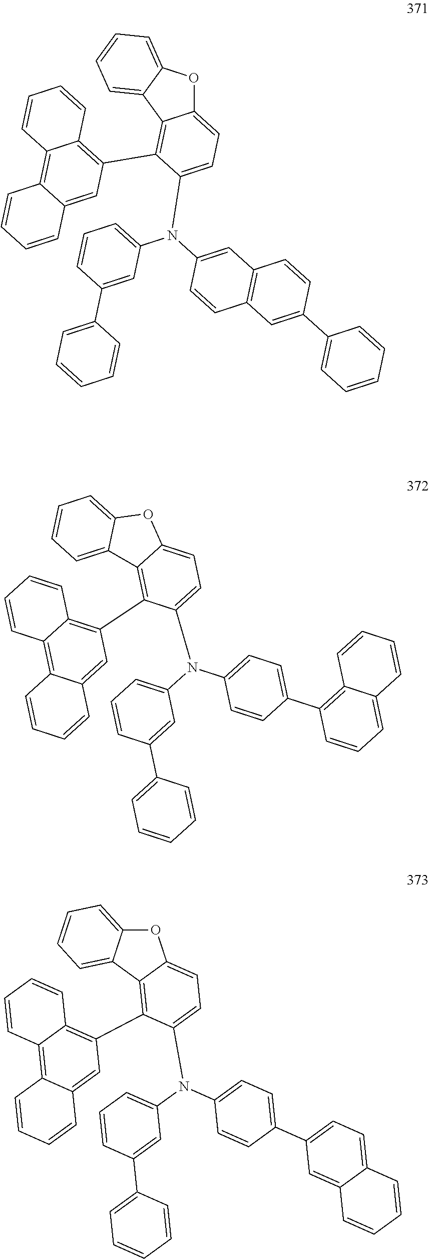 [SMILES: c1ccc(-c2cccc(N(c3ccc4cc(-c5ccccc5)ccc4c3)c3ccc4oc5ccccc5c4c3-c3cc4ccccc4c4ccccc34)c2)cc1 ; c1ccc(-c2cccc(N(c3ccc(-c4cccc5ccccc45)cc3)c3ccc4oc5ccccc5c4c3-c3cc4ccccc4c4ccccc34)c2)cc1 ; c1ccc(-c2cccc(N(c3ccc(-c4ccc5ccccc5c4)cc3)c3ccc4oc5ccccc5c4c3-c3cc4ccccc4c4ccccc34)c2)cc1]

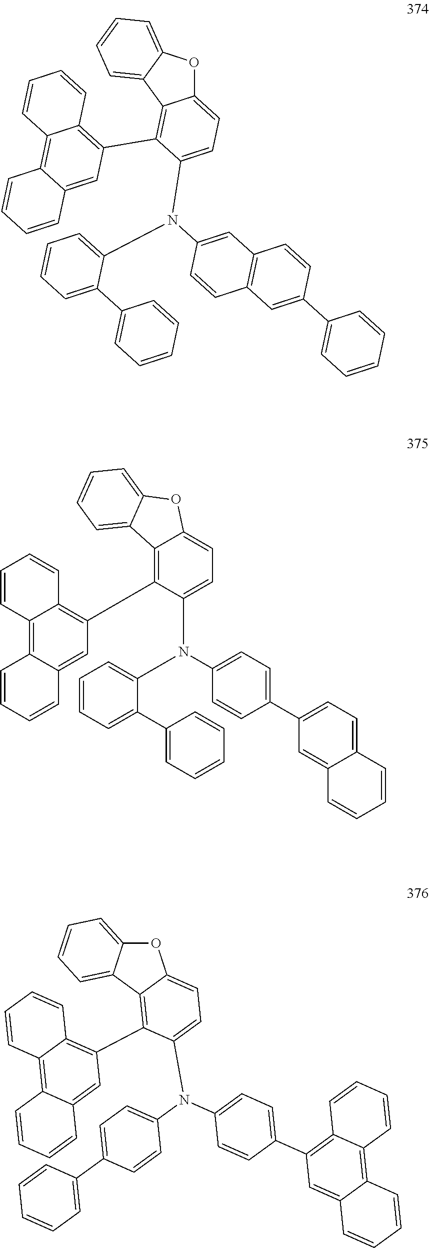 [SMILES: c1ccc(-c2ccc3cc(N(c4ccccc4-c4ccccc4)c4ccc5oc6ccccc6c5c4-c4cc5ccccc5c5ccccc45)ccc3c2)cc1 ; c1ccc(-c2ccccc2N(c2ccc(-c3ccc4ccccc4c3)cc2)c2ccc3oc4ccccc4c3c2-c2cc3ccccc3c3ccccc23)cc1 ; c1ccc(-c2ccc(N(c3ccc(-c4cc5ccccc5c5ccccc45)cc3)c3ccc4oc5ccccc5c4c3-c3cc4ccccc4c4ccccc34)cc2)cc1]

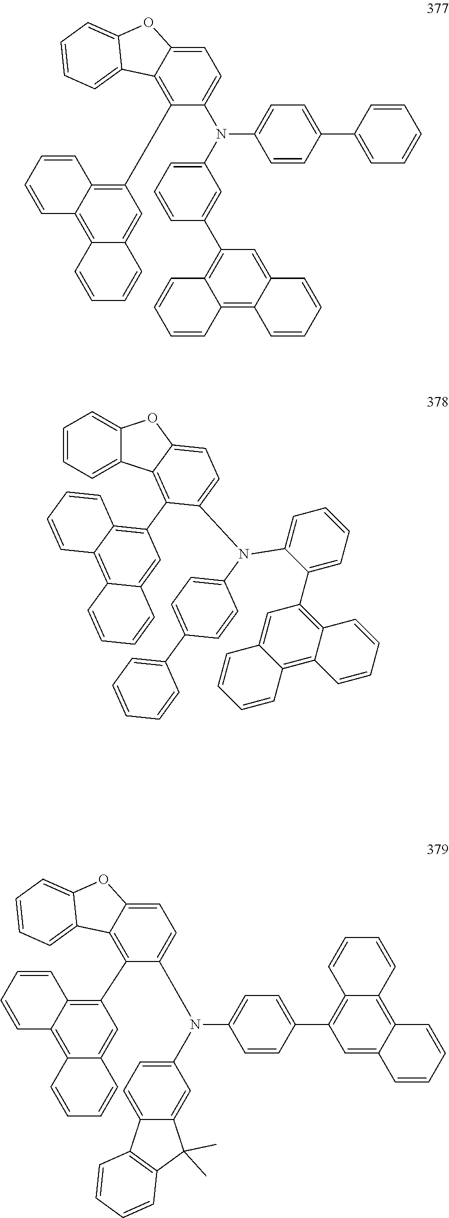 [SMILES: c1ccc(-c2ccc(N(c3cccc(-c4cc5ccccc5c5ccccc45)c3)c3ccc4oc5ccccc5c4c3-c3cc4ccccc4c4ccccc34)cc2)cc1 ; c1ccc(-c2ccc(N(c3ccccc3-c3cc4ccccc4c4ccccc34)c3ccc4oc5ccccc5c4c3-c3cc4ccccc4c4ccccc34)cc2)cc1 ; CC1(C)c2ccccc2-c2ccc(N(c3ccc(-c4cc5ccccc5c5ccccc45)cc3)c3ccc4oc5ccccc5c4c3-c3cc4ccccc4c4ccccc34)cc21]

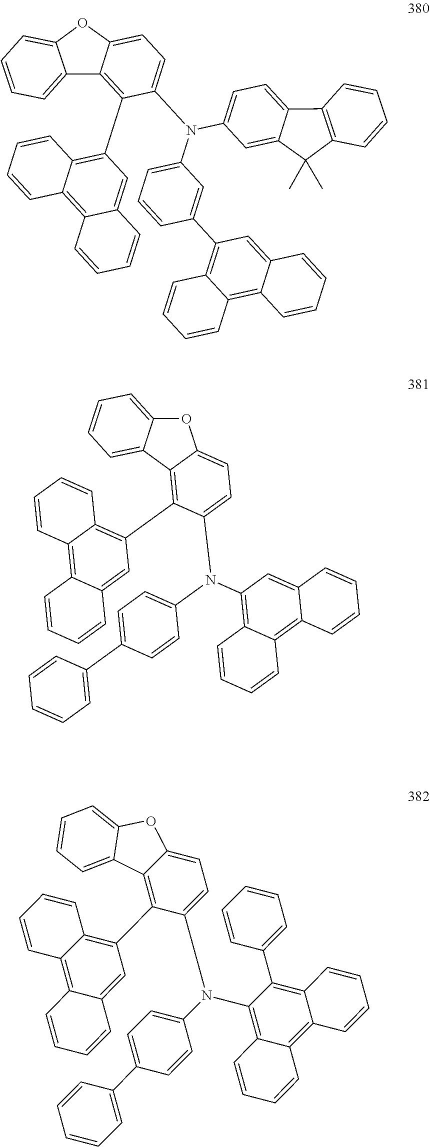 [SMILES: CC1(C)c2ccccc2-c2ccc(N(c3cccc(-c4cc5ccccc5c5ccccc45)c3)c3ccc4oc5ccccc5c4c3-c3cc4ccccc4c4ccccc34)cc21 ; c1ccc(-c2ccc(N(c3ccc4oc5ccccc5c4c3-c3cc4ccccc4c4ccccc34)c3cc4ccccc4c4ccccc34)cc2)cc1 ; c1ccc(-c2ccc(N(c3ccc4oc5ccccc5c4c3-c3cc4ccccc4c4ccccc34)c3c(-c4ccccc4)c4ccccc4c4ccccc34)cc2)cc1]

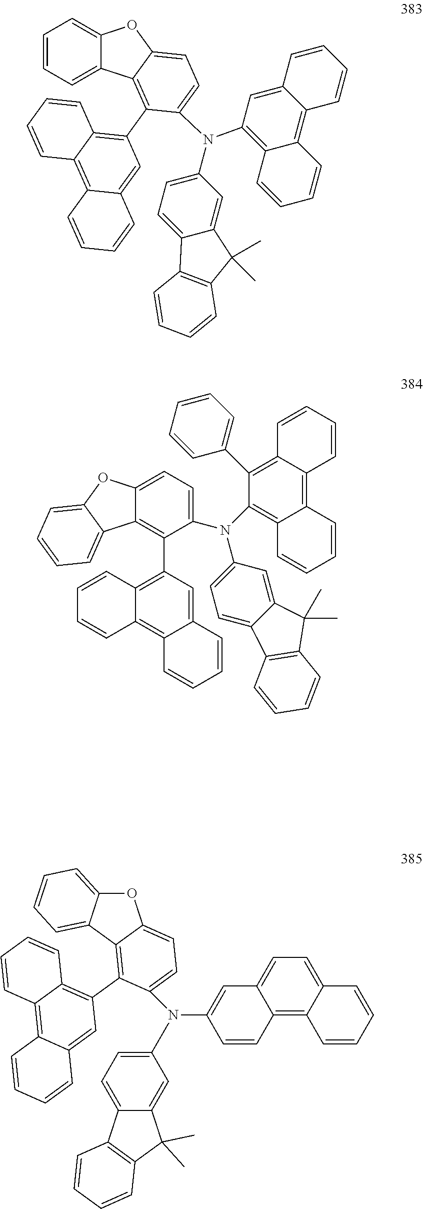 [SMILES: CC1(C)c2ccccc2-c2ccc(N(c3ccc4oc5ccccc5c4c3-c3cc4ccccc4c4ccccc34)c3cc4ccccc4c4ccccc34)cc21 ; CC1(C)c2ccccc2-c2ccc(N(c3ccc4oc5ccccc5c4c3-c3cc4ccccc4c4ccccc34)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3cc4ccccc4c4ccccc34)cc21]

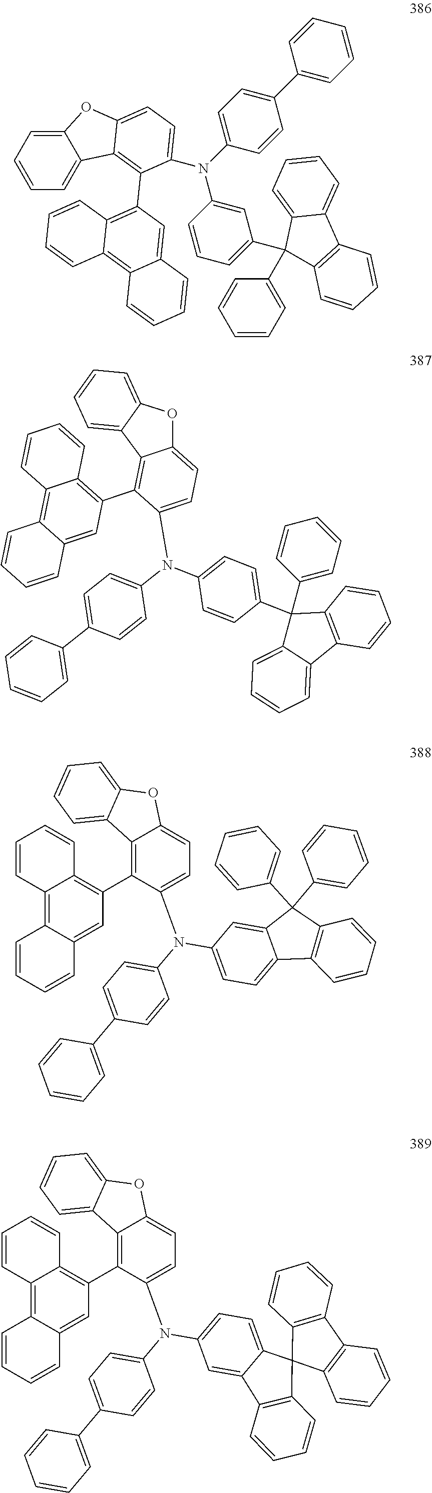 [SMILES: c1ccc(-c2ccc(N(c3cccc(C4(c5ccccc5)c5ccccc5-c5ccccc54)c3)c3ccc4oc5ccccc5c4c3-c3cc4ccccc4c4ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cc4ccccc4c4ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3cc4ccccc4c4ccccc34)cc2)cc1 ; c1ccc(-c2ccc(N(c3ccc4c(c3)-c3ccccc3C43c4ccccc4-c4ccccc43)c3ccc4oc5ccccc5c4c3-c3cc4ccccc4c4ccccc34)cc2)cc1]

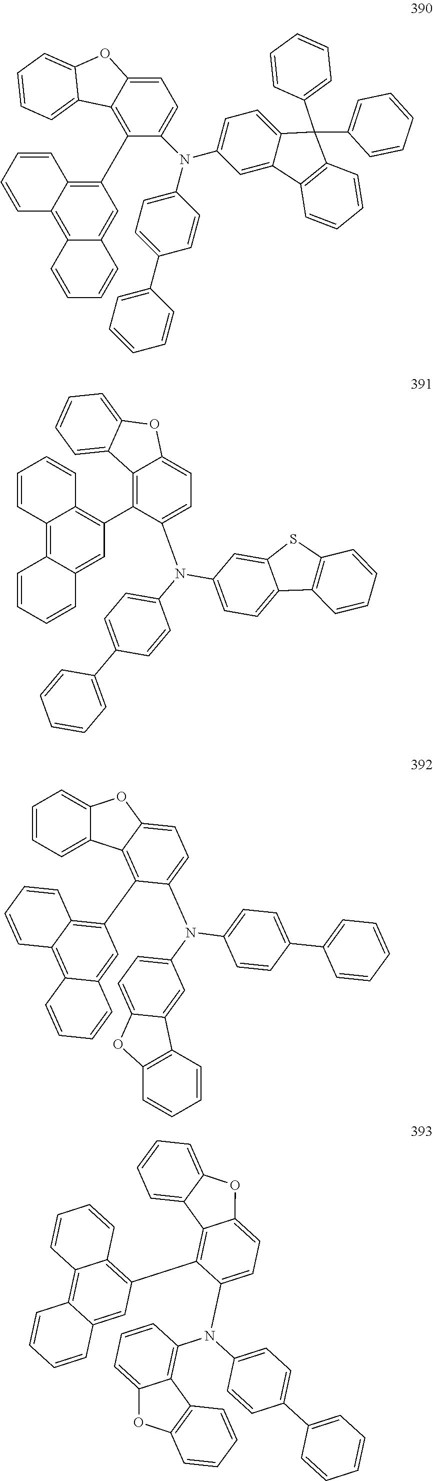

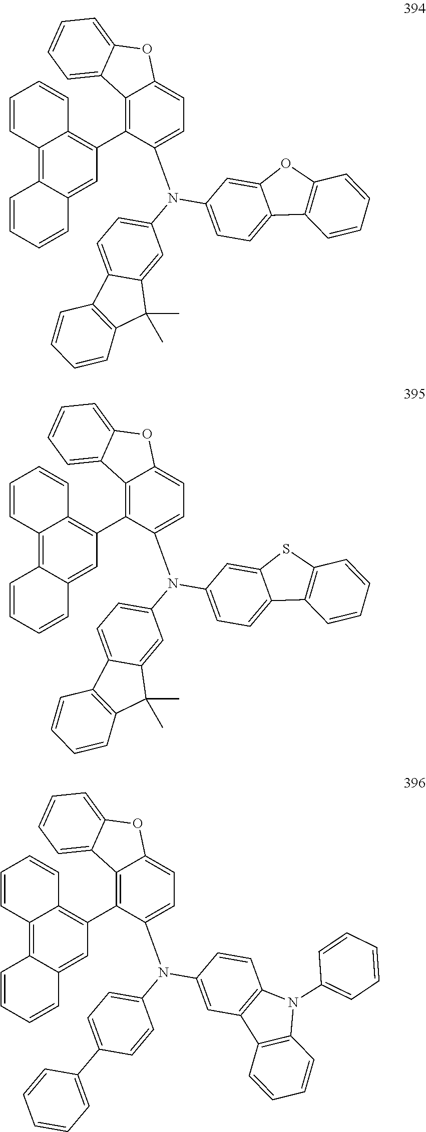

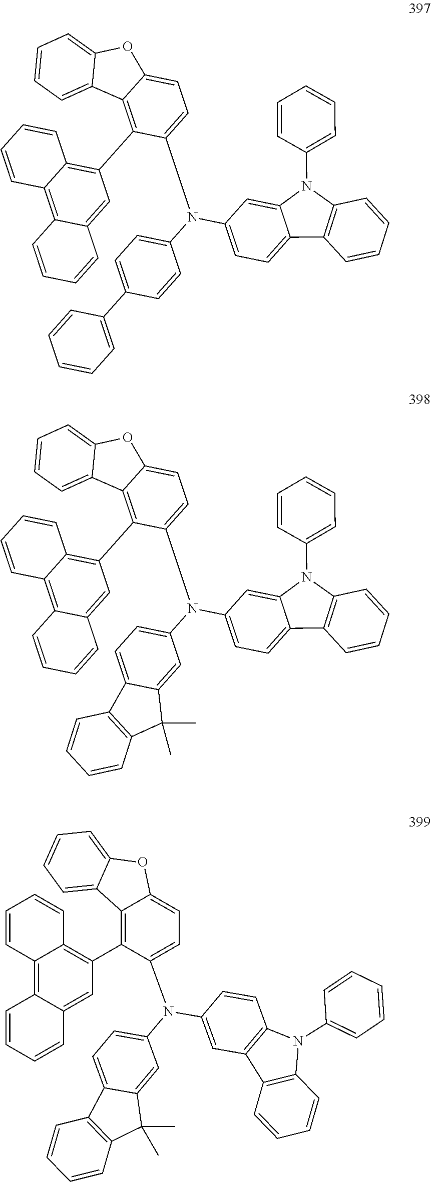 [SMILES: c1ccc(-c2ccc(N(c3ccc4c5ccccc5n(-c5ccccc5)c4c3)c3ccc4oc5ccccc5c4c3-c3cc4ccccc4c4ccccc34)cc2)cc1 ; CC1(C)c2ccccc2-c2ccc(N(c3ccc4c5ccccc5n(-c5ccccc5)c4c3)c3ccc4oc5ccccc5c4c3-c3cc4ccccc4c4ccccc34)cc21 ; CC1(C)c2ccccc2-c2ccc(N(c3ccc4c(c3)c3ccccc3n4-c3ccccc3)c3ccc4oc5ccccc5c4c3-c3cc4ccccc4c4ccccc34)cc21]

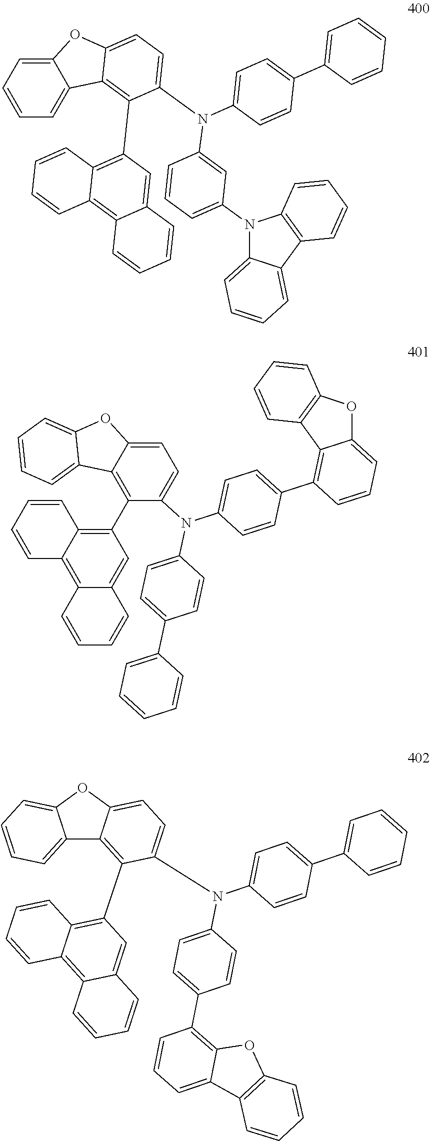

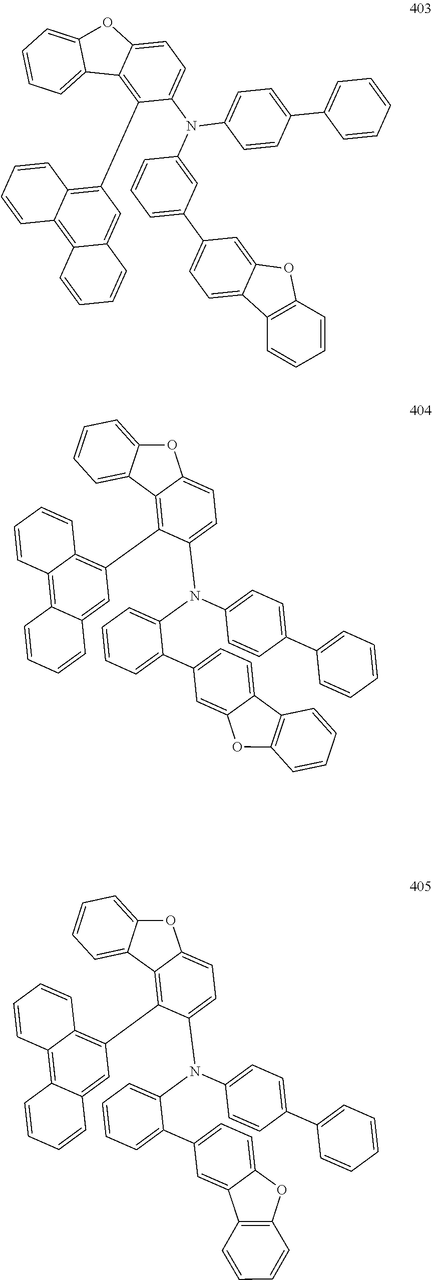 [SMILES: c1ccc(-c2ccc(N(c3cccc(-c4ccc5c(c4)oc4ccccc45)c3)c3ccc4oc5ccccc5c4c3-c3cc4ccccc4c4ccccc34)cc2)cc1 ; c1ccc(-c2ccc(N(c3ccccc3-c3ccc4c(c3)oc3ccccc34)c3ccc4oc5ccccc5c4c3-c3cc4ccccc4c4ccccc34)cc2)cc1 ; c1ccc(-c2ccc(N(c3ccccc3-c3ccc4oc5ccccc5c4c3)c3ccc4oc5ccccc5c4c3-c3cc4ccccc4c4ccccc34)cc2)cc1]

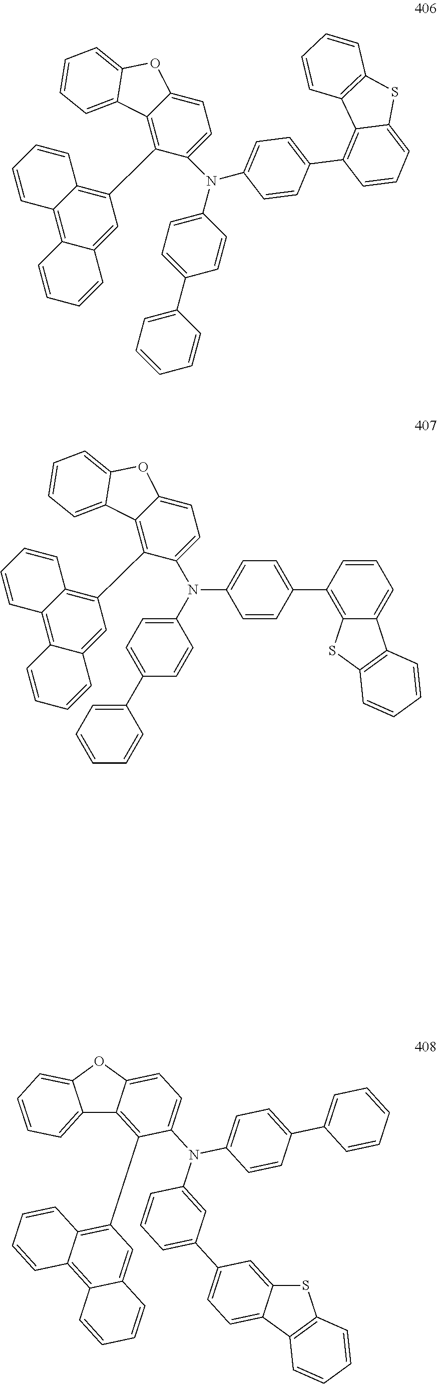 [SMILES: c1ccc(-c2ccc(N(c3ccc(-c4cccc5sc6ccccc6c45)cc3)c3ccc4oc5ccccc5c4c3-c3cc4ccccc4c4ccccc34)cc2)cc1 ; c1ccc(-c2ccc(N(c3ccc(-c4cccc5c4sc4ccccc45)cc3)c3ccc4oc5ccccc5c4c3-c3cc4ccccc4c4ccccc34)cc2)cc1 ; c1ccc(-c2ccc(N(c3cccc(-c4ccc5c(c4)sc4ccccc45)c3)c3ccc4oc5ccccc5c4c3-c3cc4ccccc4c4ccccc34)cc2)cc1]

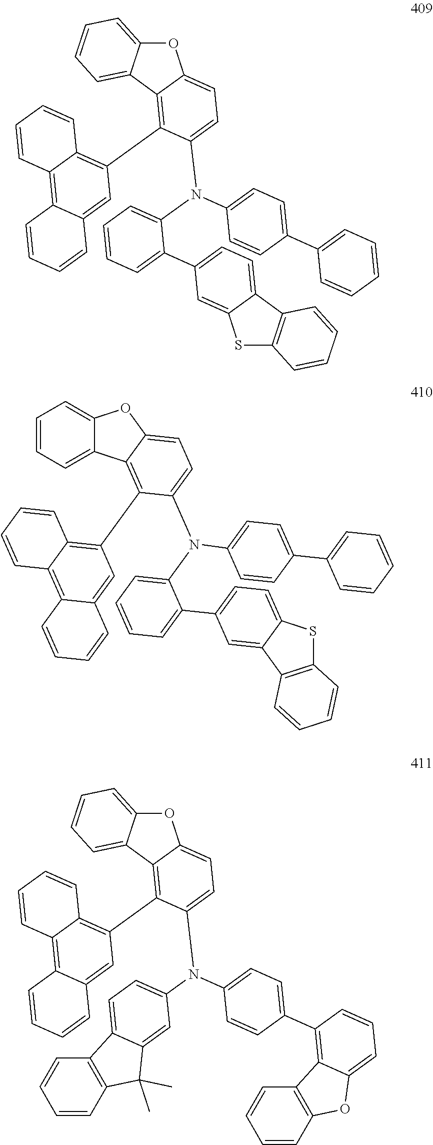 [SMILES: c1ccc(-c2ccc(N(c3ccccc3-c3ccc4c(c3)sc3ccccc34)c3ccc4oc5ccccc5c4c3-c3cc4ccccc4c4ccccc34)cc2)cc1 ; c1ccc(-c2ccc(N(c3ccccc3-c3ccc4sc5ccccc5c4c3)c3ccc4oc5ccccc5c4c3-c3cc4ccccc4c4ccccc34)cc2)cc1 ; CC1(C)c2ccccc2-c2ccc(N(c3ccc(-c4cccc5oc6ccccc6c45)cc3)c3ccc4oc5ccccc5c4c3-c3cc4ccccc4c4ccccc34)cc21]

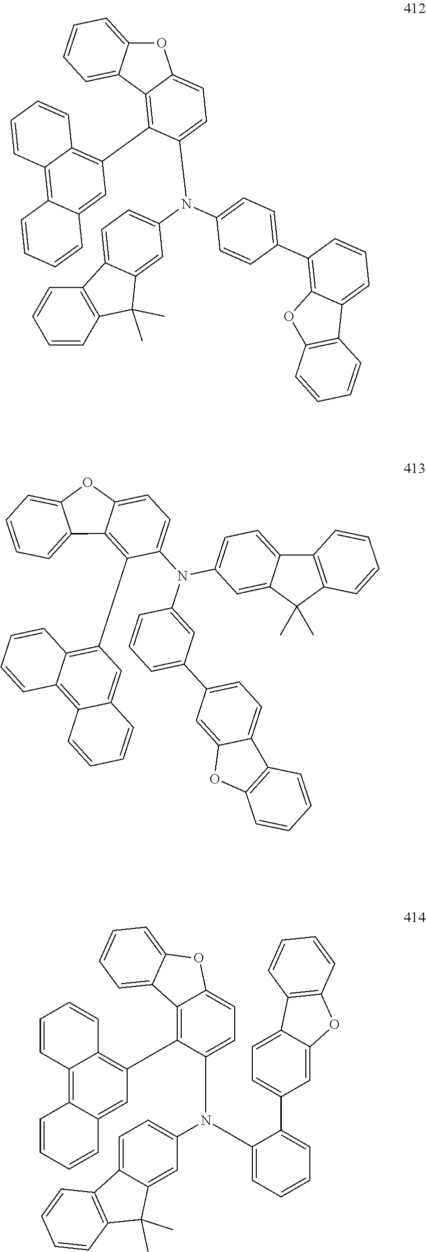 [SMILES: CC1(C)c2ccccc2-c2ccc(N(c3ccc(-c4cccc5c4oc4ccccc45)cc3)c3ccc4oc5ccccc5c4c3-c3cc4ccccc4c4ccccc34)cc21 ; CC1(C)c2ccccc2-c2ccc(N(c3cccc(-c4ccc5c(c4)oc4ccccc45)c3)c3ccc4oc5ccccc5c4c3-c3cc4ccccc4c4ccccc34)cc21 ; CC1(C)c2ccccc2-c2ccc(N(c3ccccc3-c3ccc4c(c3)oc3ccccc34)c3ccc4oc5ccccc5c4c3-c3cc4ccccc4c4ccccc34)cc21]

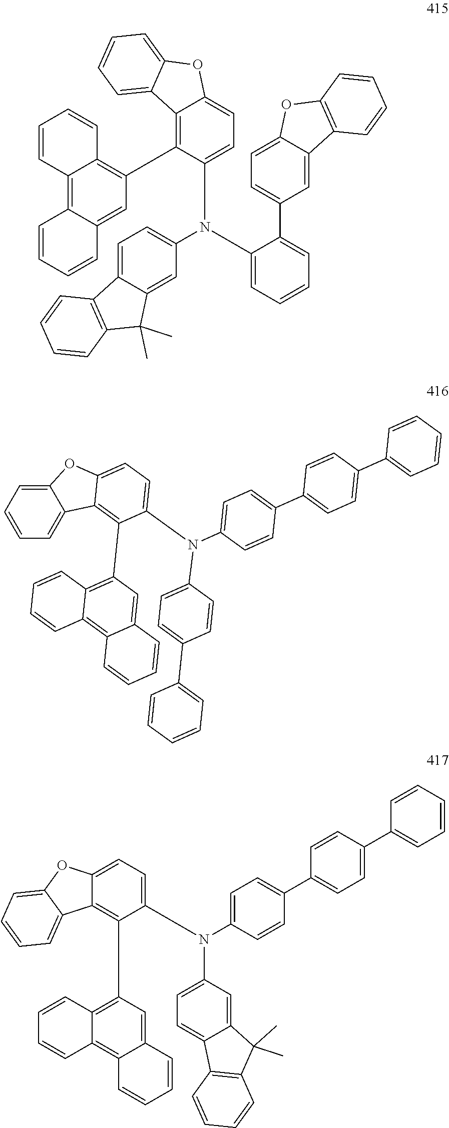 [SMILES: CC1(C)c2ccccc2-c2ccc(N(c3ccccc3-c3ccc4oc5ccccc5c4c3)c3ccc4oc5ccccc5c4c3-c3cc4ccccc4c4ccccc34)cc21 ; c1ccc(-c2ccc(-c3ccc(N(c4ccc(-c5ccccc5)cc4)c4ccc5oc6ccccc6c5c4-c4cc5ccccc5c5ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cc4ccccc4c4ccccc34)cc21]

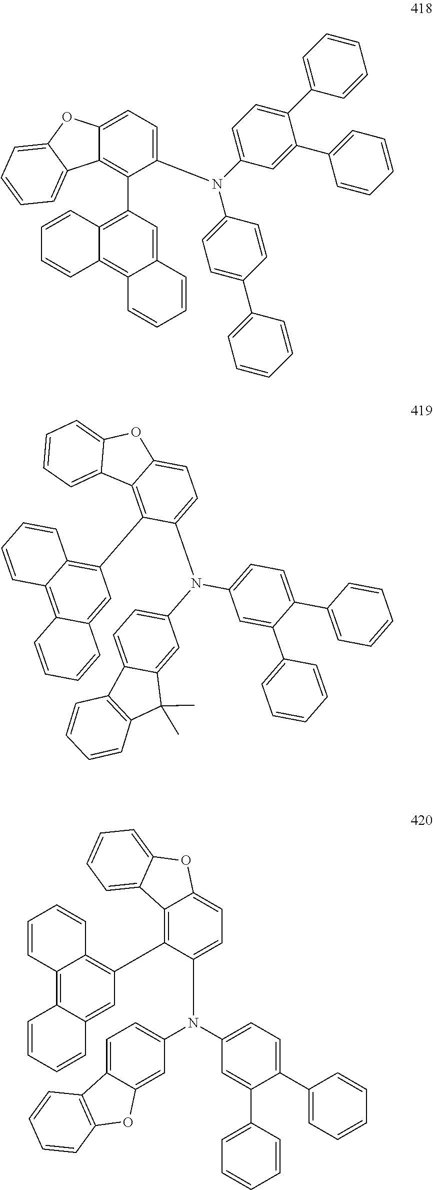 [SMILES: c1ccc(-c2ccc(N(c3ccc(-c4ccccc4)c(-c4ccccc4)c3)c3ccc4oc5ccccc5c4c3-c3cc4ccccc4c4ccccc34)cc2)cc1 ; CC1(C)c2ccccc2-c2ccc(N(c3ccc(-c4ccccc4)c(-c4ccccc4)c3)c3ccc4oc5ccccc5c4c3-c3cc4ccccc4c4ccccc34)cc21 ; c1ccc(-c2ccc(N(c3ccc4c(c3)oc3ccccc34)c3ccc4oc5ccccc5c4c3-c3cc4ccccc4c4ccccc34)cc2-c2ccccc2)cc1]

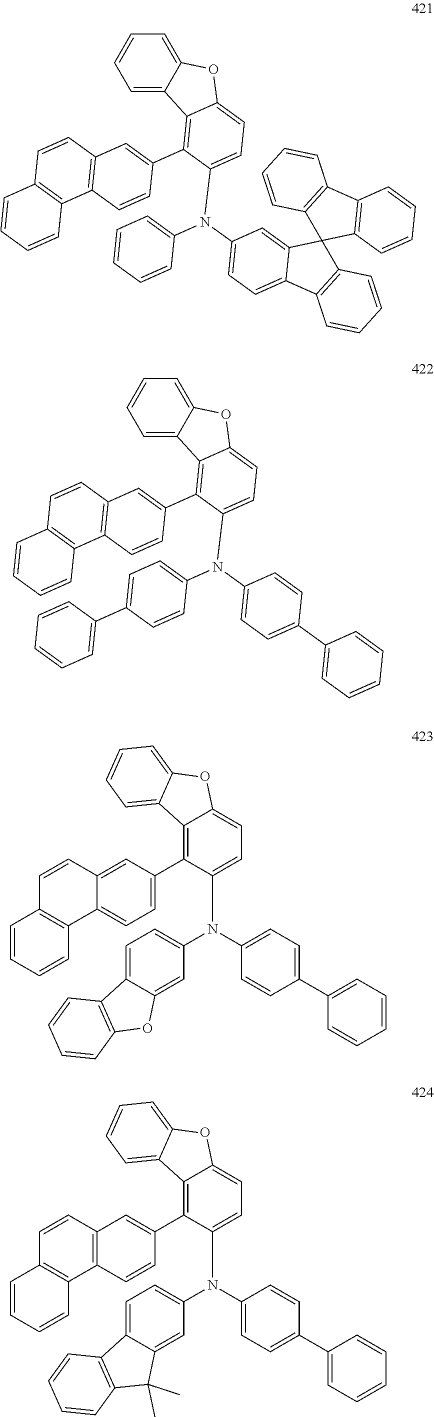 [SMILES: c1ccc(N(c2ccc3c(c2)C2(c4ccccc4-c4ccccc42)c2ccccc2-3)c2ccc3oc4ccccc4c3c2-c2ccc3c(ccc4ccccc43)c2)cc1 ; c1ccc(-c2ccc(N(c3ccc(-c4ccccc4)cc3)c3ccc4oc5ccccc5c4c3-c3ccc4c(ccc5ccccc54)c3)cc2)cc1 ; c1ccc(-c2ccc(N(c3ccc4c(c3)oc3ccccc34)c3ccc4oc5ccccc5c4c3-c3ccc4c(ccc5ccccc54)c3)cc2)cc1 ; CC1(C)c2ccccc2-c2ccc(N(c3ccc(-c4ccccc4)cc3)c3ccc4oc5ccccc5c4c3-c3ccc4c(ccc5ccccc54)c3)cc21]

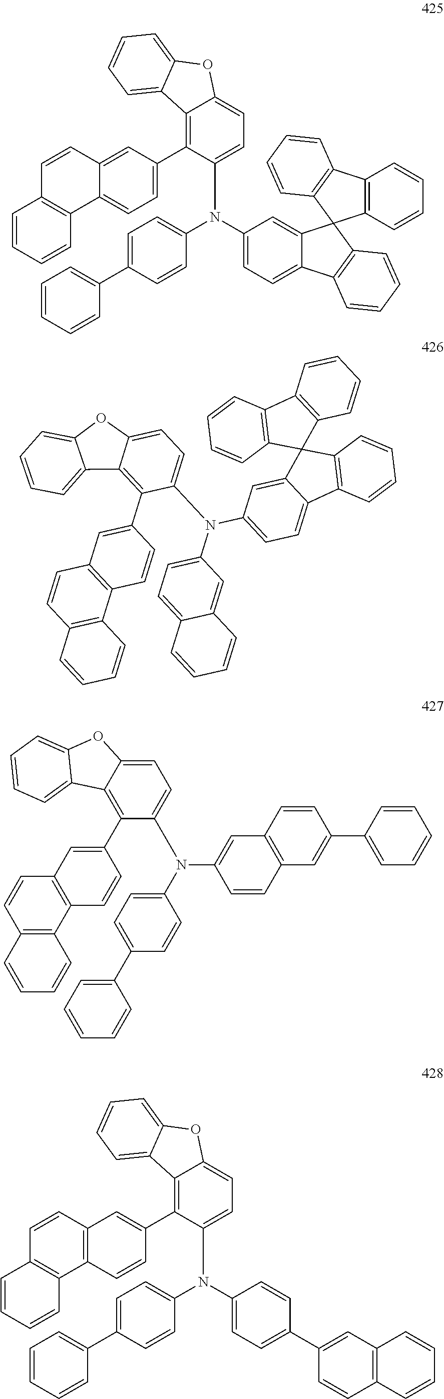 [SMILES: c1ccc(-c2ccc(N(c3ccc4c(c3)C3(c5ccccc5-c5ccccc53)c3ccccc3-4)c3ccc4oc5ccccc5c4c3-c3ccc4c(ccc5ccccc54)c3)cc2)cc1 ; c1ccc2c(c1)-c1ccccc1C21c2ccccc2-c2ccc(N(c3ccc4ccccc4c3)c3ccc4oc5ccccc5c4c3-c3ccc4c(ccc5ccccc54)c3)cc21 ; c1ccc(-c2ccc(N(c3ccc4cc(-c5ccccc5)ccc4c3)c3ccc4oc5ccccc5c4c3-c3ccc4c(ccc5ccccc54)c3)cc2)cc1 ; c1ccc(-c2ccc(N(c3ccc(-c4ccc5ccccc5c4)cc3)c3ccc4oc5ccccc5c4c3-c3ccc4c(ccc5ccccc54)c3)cc2)cc1]

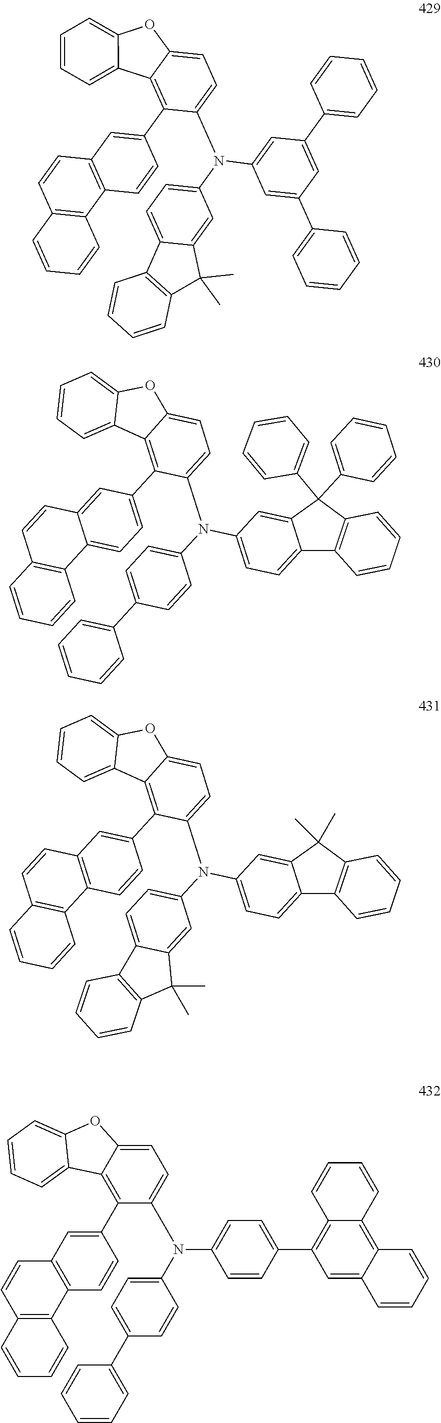

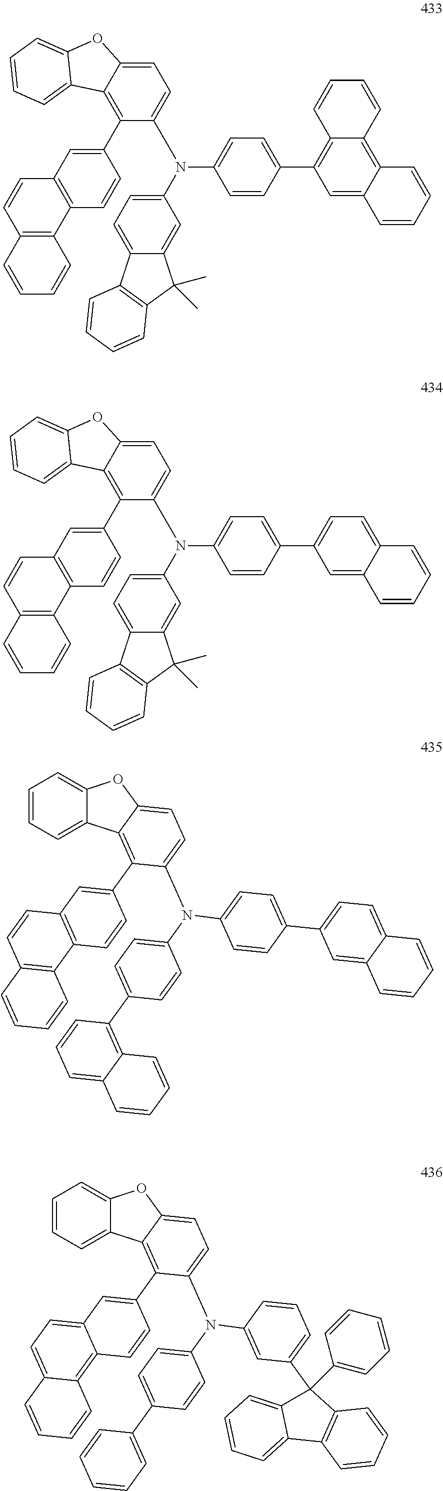 [SMILES: CC1(C)c2ccccc2-c2ccc(N(c3ccc(-c4cc5ccccc5c5ccccc45)cc3)c3ccc4oc5ccccc5c4c3-c3ccc4c(ccc5ccccc54)c3)cc21 ; CC1(C)c2ccccc2-c2ccc(N(c3ccc(-c4ccc5ccccc5c4)cc3)c3ccc4oc5ccccc5c4c3-c3ccc4c(ccc5ccccc54)c3)cc21 ; c1ccc2cc(-c3ccc(N(c4ccc(-c5cccc6ccccc56)cc4)c4ccc5oc6ccccc6c5c4-c4ccc5c(ccc6ccccc65)c4)cc3)ccc2c1 ; c1ccc(-c2ccc(N(c3cccc(C4(c5ccccc5)c5ccccc5-c5ccccc54)c3)c3ccc4oc5ccccc5c4c3-c3ccc4c(ccc5ccccc54)c3)cc2)cc1]

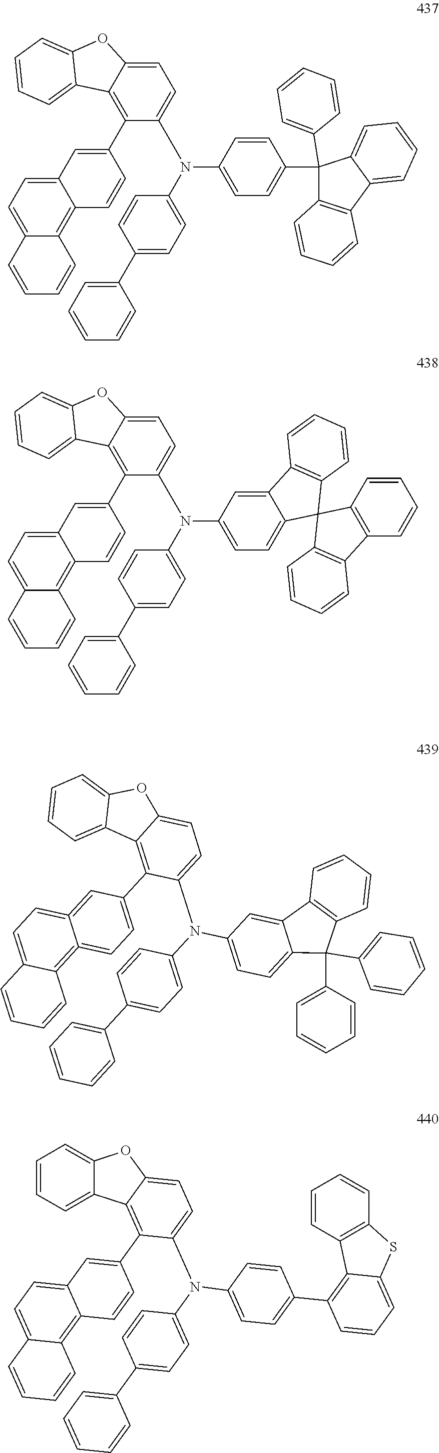

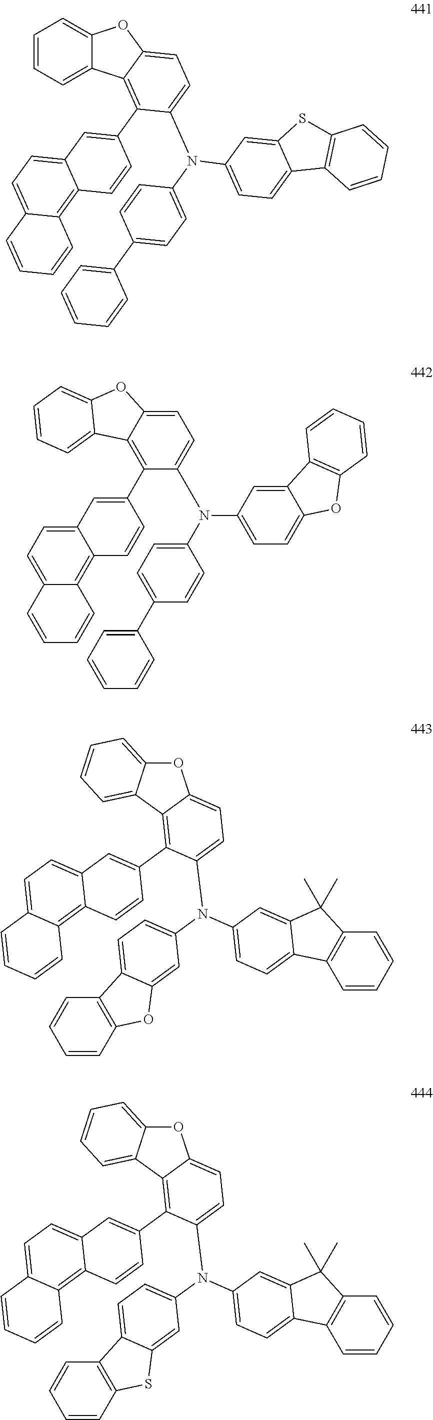 [SMILES: c1ccc(-c2ccc(N(c3ccc4c(c3)sc3ccccc34)c3ccc4oc5ccccc5c4c3-c3ccc4c(ccc5ccccc54)c3)cc2)cc1 ; c1ccc(-c2ccc(N(c3ccc4oc5ccccc5c4c3)c3ccc4oc5ccccc5c4c3-c3ccc4c(ccc5ccccc54)c3)cc2)cc1 ; CC1(C)c2ccccc2-c2ccc(N(c3ccc4c(c3)oc3ccccc34)c3ccc4oc5ccccc5c4c3-c3ccc4c(ccc5ccccc54)c3)cc21 ; CC1(C)c2ccccc2-c2ccc(N(c3ccc4c(c3)sc3ccccc34)c3ccc4oc5ccccc5c4c3-c3ccc4c(ccc5ccccc54)c3)cc21]

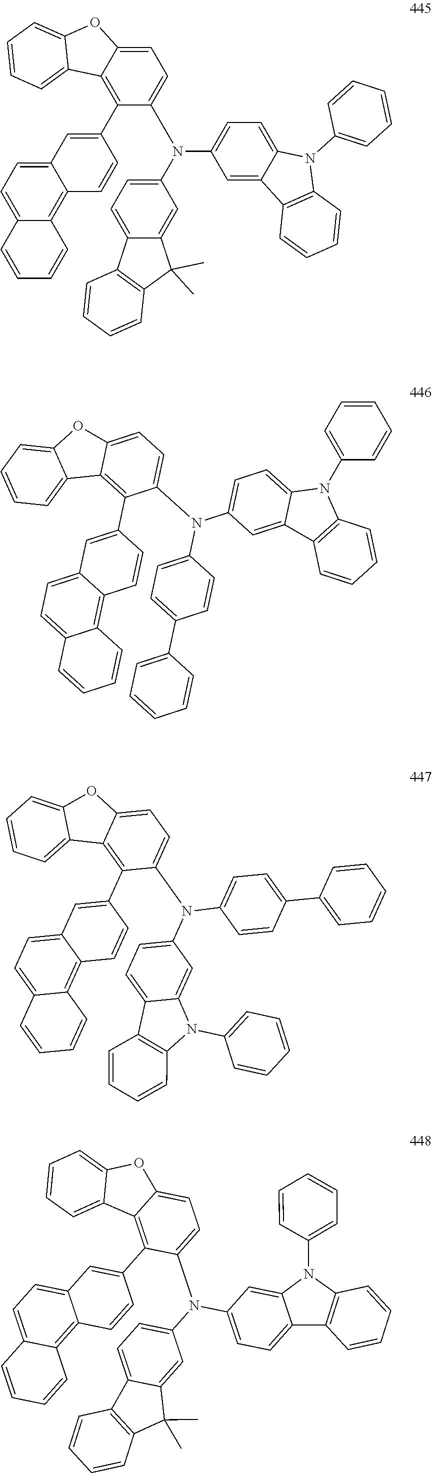 [SMILES: CC1(C)c2ccccc2-c2ccc(N(c3ccc4c(c3)c3ccccc3n4-c3ccccc3)c3ccc4oc5ccccc5c4c3-c3ccc4c(ccc5ccccc54)c3)cc21 ; c1ccc(-c2ccc(N(c3ccc4c(c3)c3ccccc3n4-c3ccccc3)c3ccc4oc5ccccc5c4c3-c3ccc4c(ccc5ccccc54)c3)cc2)cc1 ; c1ccc(-c2ccc(N(c3ccc4c5ccccc5n(-c5ccccc5)c4c3)c3ccc4oc5ccccc5c4c3-c3ccc4c(ccc5ccccc54)c3)cc2)cc1 ; CC1(C)c2ccccc2-c2ccc(N(c3ccc4c5ccccc5n(-c5ccccc5)c4c3)c3ccc4oc5ccccc5c4c3-c3ccc4c(ccc5ccccc54)c3)cc21]

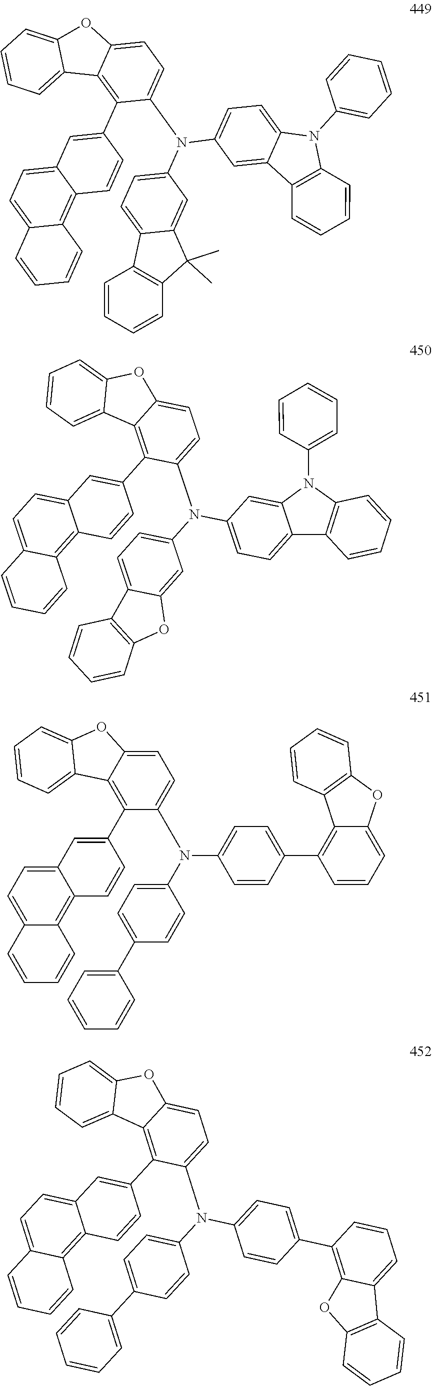

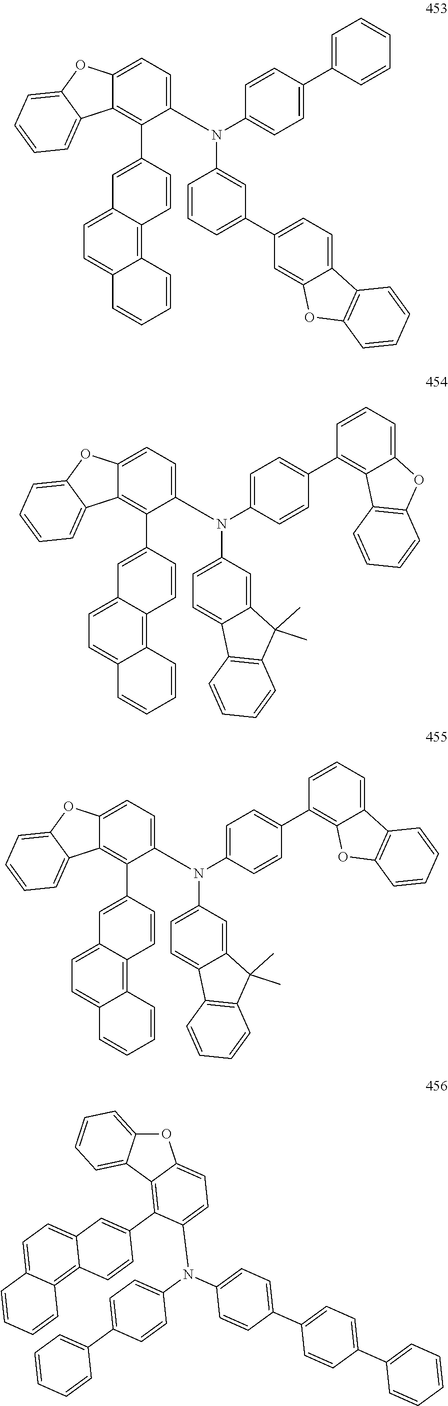 [SMILES: c1ccc(-c2ccc(N(c3cccc(-c4ccc5c(c4)oc4ccccc45)c3)c3ccc4oc5ccccc5c4c3-c3ccc4c(ccc5ccccc54)c3)cc2)cc1 ; CC1(C)c2ccccc2-c2ccc(N(c3ccc(-c4cccc5oc6ccccc6c45)cc3)c3ccc4oc5ccccc5c4c3-c3ccc4c(ccc5ccccc54)c3)cc21 ; CC1(C)c2ccccc2-c2ccc(N(c3ccc(-c4cccc5c4oc4ccccc45)cc3)c3ccc4oc5ccccc5c4c3-c3ccc4c(ccc5ccccc54)c3)cc21 ; c1ccc(-c2ccc(-c3ccc(N(c4ccc(-c5ccccc5)cc4)c4ccc5oc6ccccc6c5c4-c4ccc5c(ccc6ccccc65)c4)cc3)cc2)cc1]

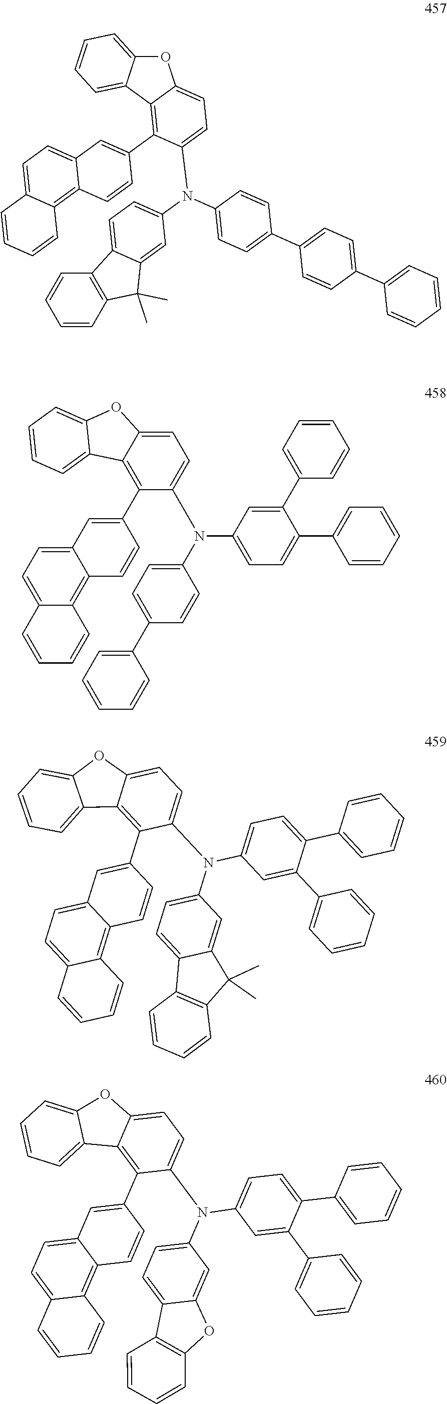

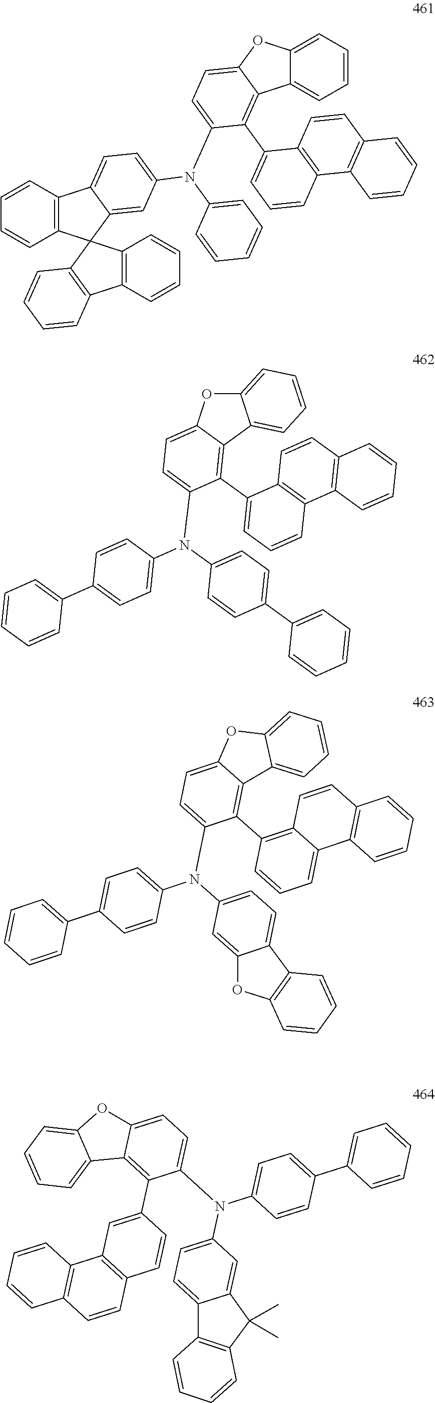

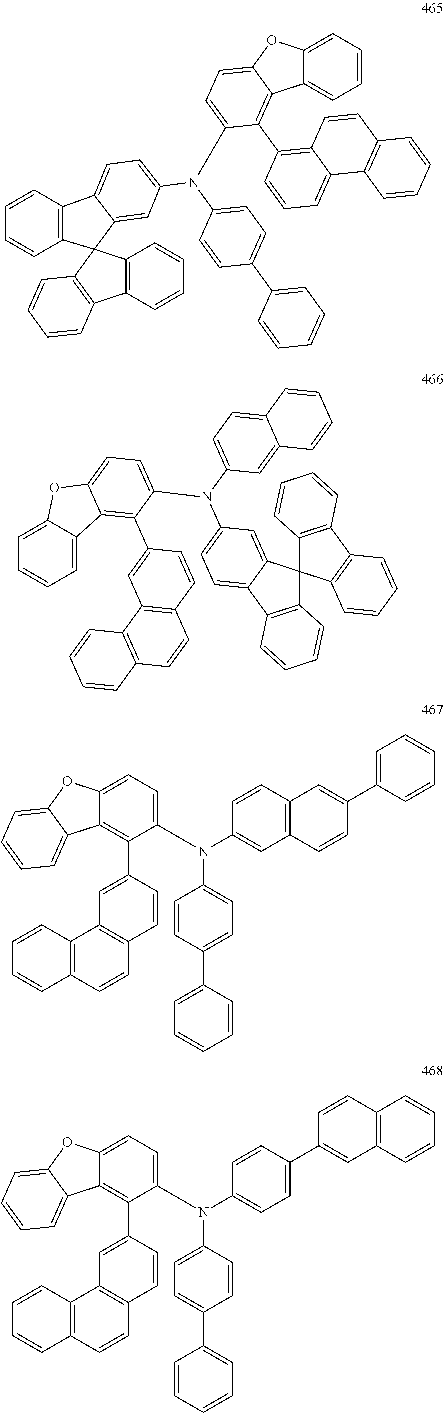

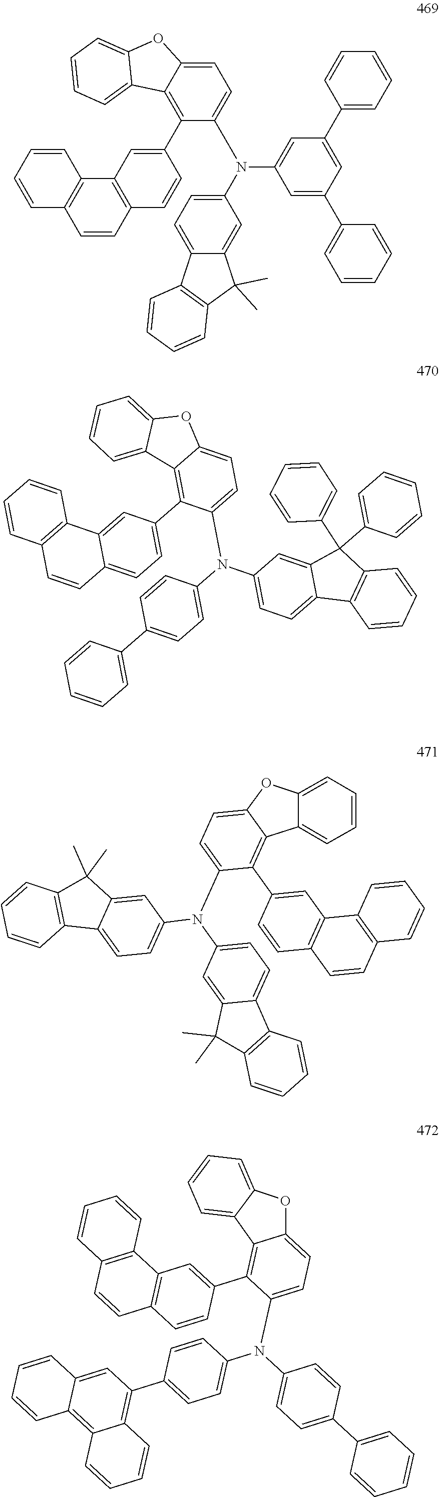 [SMILES: CC1(C)c2ccccc2-c2ccc(N(c3cc(-c4ccccc4)cc(-c4ccccc4)c3)c3ccc4oc5ccccc5c4c3-c3ccc4ccc5ccccc5c4c3)cc21 ; c1ccc(-c2ccc(N(c3ccc4c(c3)C(c3ccccc3)(c3ccccc3)c3ccccc3-4)c3ccc4oc5ccccc5c4c3-c3ccc4ccc5ccccc5c4c3)cc2)cc1 ; CC1(C)c2ccccc2-c2ccc(N(c3ccc4c(c3)C(C)(C)c3ccccc3-4)c3ccc4oc5ccccc5c4c3-c3ccc4ccc5ccccc5c4c3)cc21 ; c1ccc(-c2ccc(N(c3ccc(-c4cc5ccccc5c5ccccc45)cc3)c3ccc4oc5ccccc5c4c3-c3ccc4ccc5ccccc5c4c3)cc2)cc1]

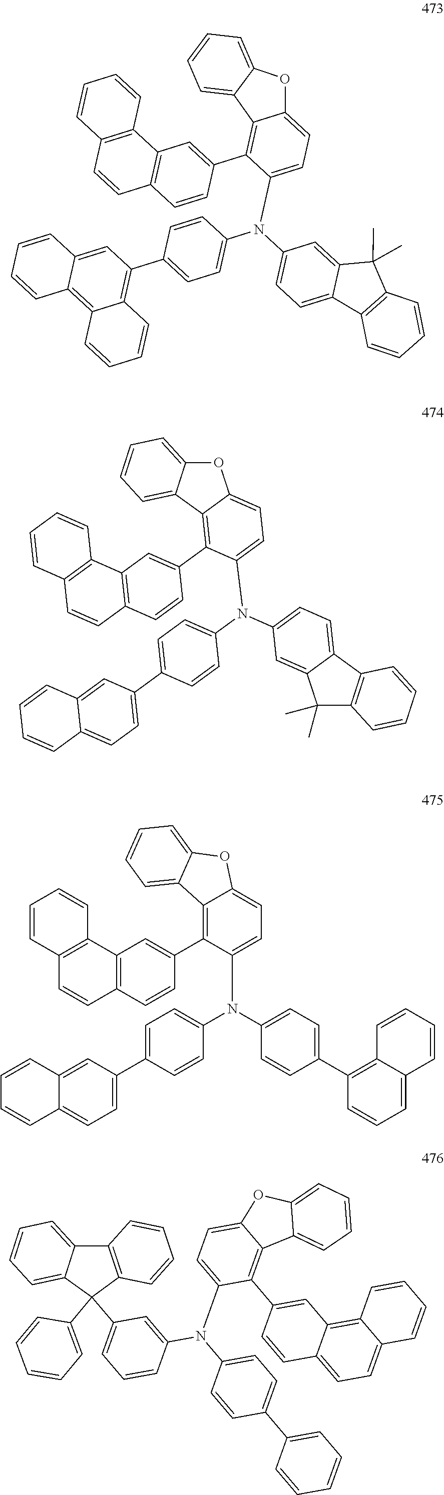 [SMILES: CC1(C)c2ccccc2-c2ccc(N(c3ccc(-c4cc5ccccc5c5ccccc45)cc3)c3ccc4oc5ccccc5c4c3-c3ccc4ccc5ccccc5c4c3)cc21 ; CC1(C)c2ccccc2-c2ccc(N(c3ccc(-c4ccc5ccccc5c4)cc3)c3ccc4oc5ccccc5c4c3-c3ccc4ccc5ccccc5c4c3)cc21 ; c1ccc2cc(-c3ccc(N(c4ccc(-c5cccc6ccccc56)cc4)c4ccc5oc6ccccc6c5c4-c4ccc5ccc6ccccc6c5c4)cc3)ccc2c1 ; c1ccc(-c2ccc(N(c3cccc(C4(c5ccccc5)c5ccccc5-c5ccccc54)c3)c3ccc4oc5ccccc5c4c3-c3ccc4ccc5ccccc5c4c3)cc2)cc1]

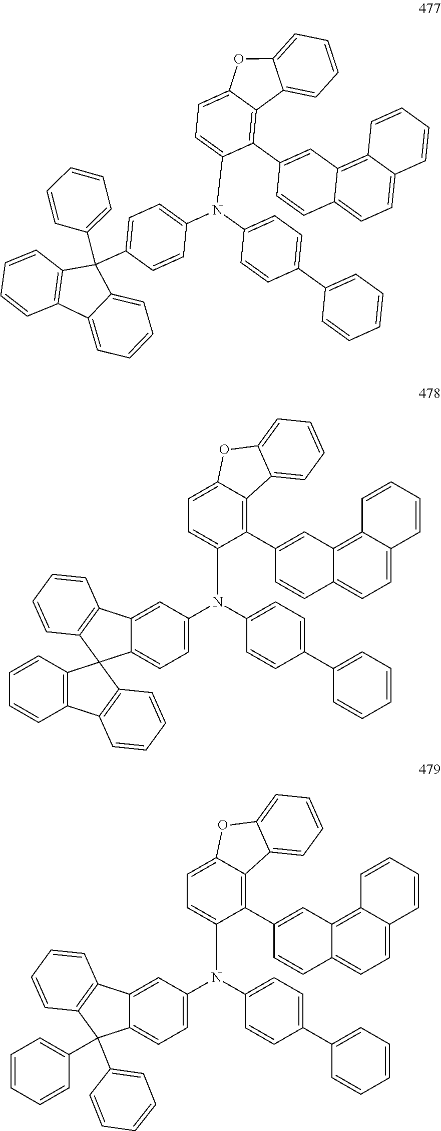 [SMILES: c1ccc(-c2ccc(N(c3ccc(C4(c5ccccc5)c5ccccc5-c5ccccc54)cc3)c3ccc4oc5ccccc5c4c3-c3ccc4ccc5ccccc5c4c3)cc2)cc1 ; c1ccc(-c2ccc(N(c3ccc4c(c3)-c3ccccc3C43c4ccccc4-c4ccccc43)c3ccc4oc5ccccc5c4c3-c3ccc4ccc5ccccc5c4c3)cc2)cc1 ; c1ccc(-c2ccc(N(c3ccc4c(c3)-c3ccccc3C4(c3ccccc3)c3ccccc3)c3ccc4oc5ccccc5c4c3-c3ccc4ccc5ccccc5c4c3)cc2)cc1]

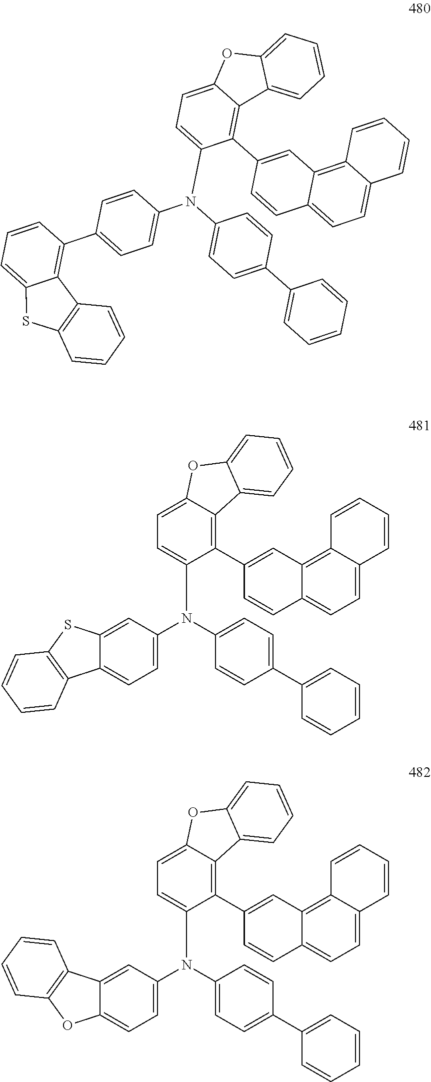 [SMILES: c1ccc(-c2ccc(N(c3ccc(-c4cccc5sc6ccccc6c45)cc3)c3ccc4oc5ccccc5c4c3-c3ccc4ccc5ccccc5c4c3)cc2)cc1 ; c1ccc(-c2ccc(N(c3ccc4c(c3)sc3ccccc34)c3ccc4oc5ccccc5c4c3-c3ccc4ccc5ccccc5c4c3)cc2)cc1 ; c1ccc(-c2ccc(N(c3ccc4oc5ccccc5c4c3)c3ccc4oc5ccccc5c4c3-c3ccc4ccc5ccccc5c4c3)cc2)cc1]

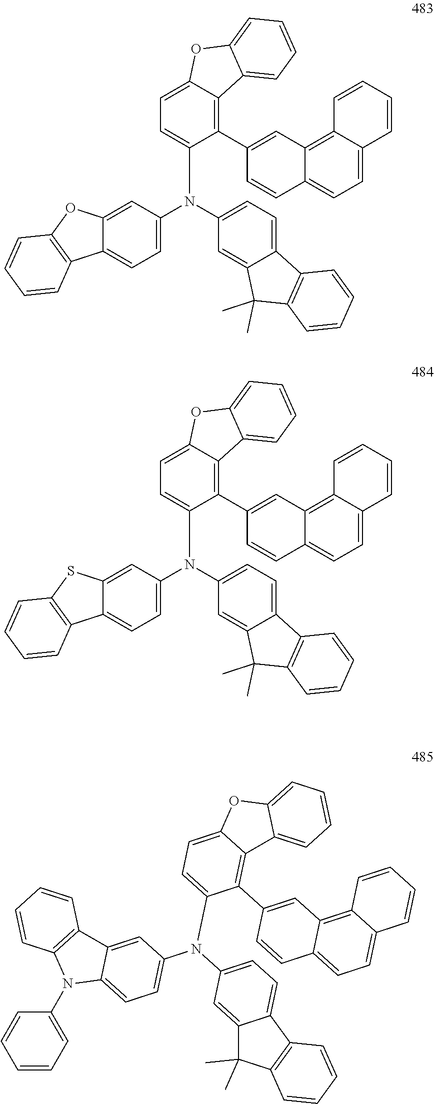 [SMILES: CC1(C)c2ccccc2-c2ccc(N(c3ccc4c(c3)oc3ccccc34)c3ccc4oc5ccccc5c4c3-c3ccc4ccc5ccccc5c4c3)cc21 ; CC1(C)c2ccccc2-c2ccc(N(c3ccc4c(c3)sc3ccccc34)c3ccc4oc5ccccc5c4c3-c3ccc4ccc5ccccc5c4c3)cc21 ; CC1(C)c2ccccc2-c2ccc(N(c3ccc4c(c3)c3ccccc3n4-c3ccccc3)c3ccc4oc5ccccc5c4c3-c3ccc4ccc5ccccc5c4c3)cc21]

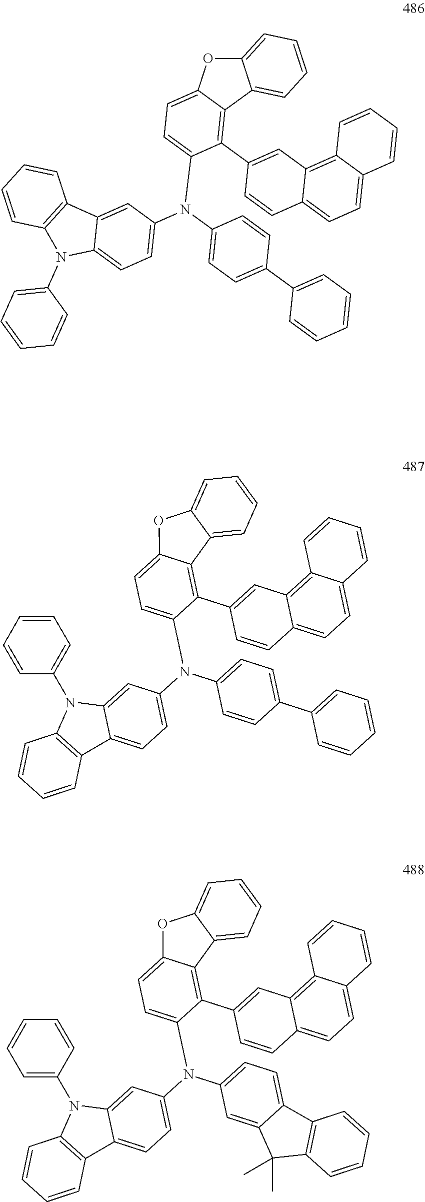 [SMILES: c1ccc(-c2ccc(N(c3ccc4c(c3)c3ccccc3n4-c3ccccc3)c3ccc4oc5ccccc5c4c3-c3ccc4ccc5ccccc5c4c3)cc2)cc1 ; c1ccc(-c2ccc(N(c3ccc4c5ccccc5n(-c5ccccc5)c4c3)c3ccc4oc5ccccc5c4c3-c3ccc4ccc5ccccc5c4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4ccc5ccccc5c4c3)cc21]

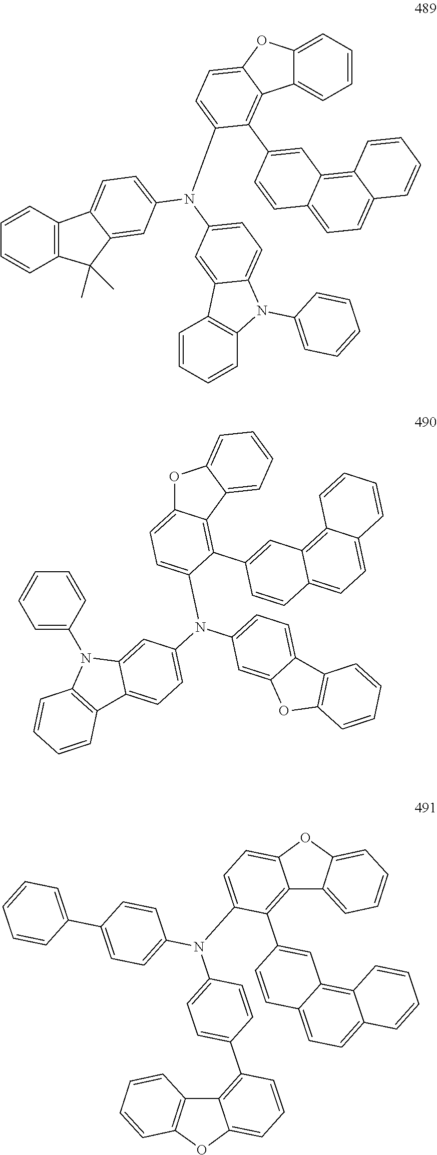 [SMILES: CC1(C)c2ccccc2-c2ccc(N(c3ccc4c(c3)c3ccccc3n4-c3ccccc3)c3ccc4oc5ccccc5c4c3-c3ccc4ccc5ccccc5c4c3)cc21 ; c1ccc(-n2c3ccccc3c3ccc(N(c4ccc5c(c4)oc4ccccc45)c4ccc5oc6ccccc6c5c4-c4ccc5ccc6ccccc6c5c4)cc32)cc1 ; c1ccc(-c2ccc(N(c3ccc(-c4cccc5oc6ccccc6c45)cc3)c3ccc4oc5ccccc5c4c3-c3ccc4ccc5ccccc5c4c3)cc2)cc1]

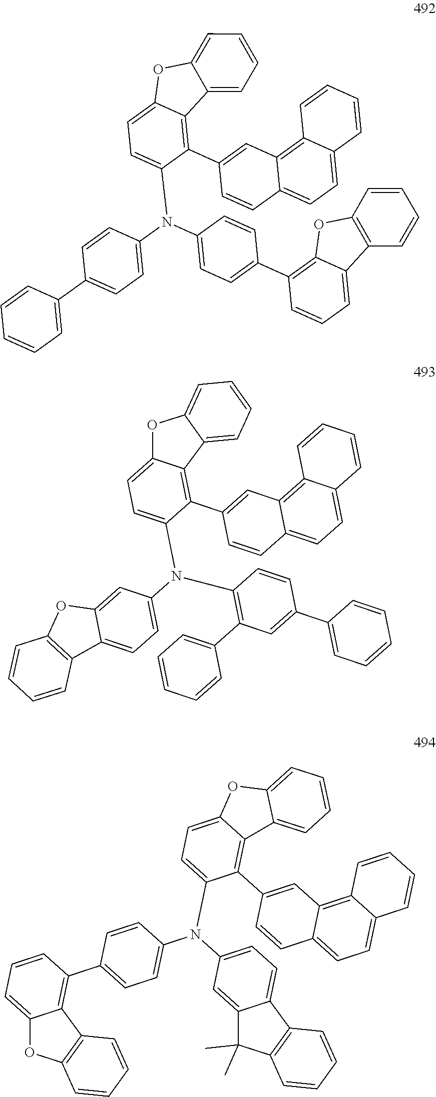 [SMILES: c1ccc(-c2ccc(N(c3ccc(-c4cccc5c4oc4ccccc45)cc3)c3ccc4oc5ccccc5c4c3-c3ccc4ccc5ccccc5c4c3)cc2)cc1 ; c1ccc(-c2ccc(N(c3ccc4c(c3)oc3ccccc34)c3ccc4oc5ccccc5c4c3-c3ccc4ccc5ccccc5c4c3)c(-c3ccccc3)c2)cc1 ; CC1(C)c2ccccc2-c2ccc(N(c3ccc(-c4cccc5oc6ccccc6c45)cc3)c3ccc4oc5ccccc5c4c3-c3ccc4ccc5ccccc5c4c3)cc21]

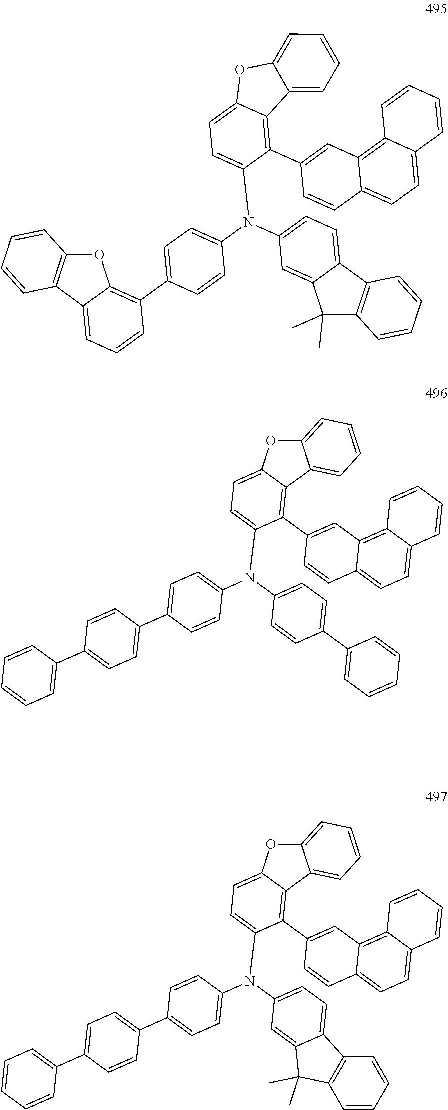 [SMILES: CC1(C)c2ccccc2-c2ccc(N(c3ccc(-c4cccc5c4oc4ccccc45)cc3)c3ccc4oc5ccccc5c4c3-c3ccc4ccc5ccccc5c4c3)cc21 ; c1ccc(-c2ccc(-c3ccc(N(c4ccc(-c5ccccc5)cc4)c4ccc5oc6ccccc6c5c4-c4ccc5ccc6ccccc6c5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4ccc5ccccc5c4c3)cc21]

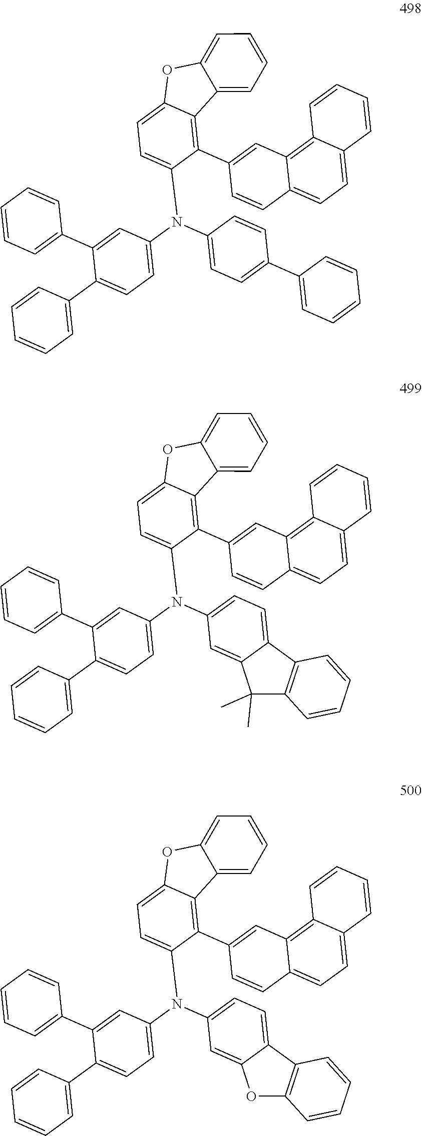 [SMILES: c1ccc(-c2ccc(N(c3ccc(-c4ccccc4)c(-c4ccccc4)c3)c3ccc4oc5ccccc5c4c3-c3ccc4ccc5ccccc5c4c3)cc2)cc1 ; CC1(C)c2ccccc2-c2ccc(N(c3ccc(-c4ccccc4)c(-c4ccccc4)c3)c3ccc4oc5ccccc5c4c3-c3ccc4ccc5ccccc5c4c3)cc21 ; c1ccc(-c2ccc(N(c3ccc4c(c3)oc3ccccc34)c3ccc4oc5ccccc5c4c3-c3ccc4ccc5ccccc5c4c3)cc2-c2ccccc2)cc1]

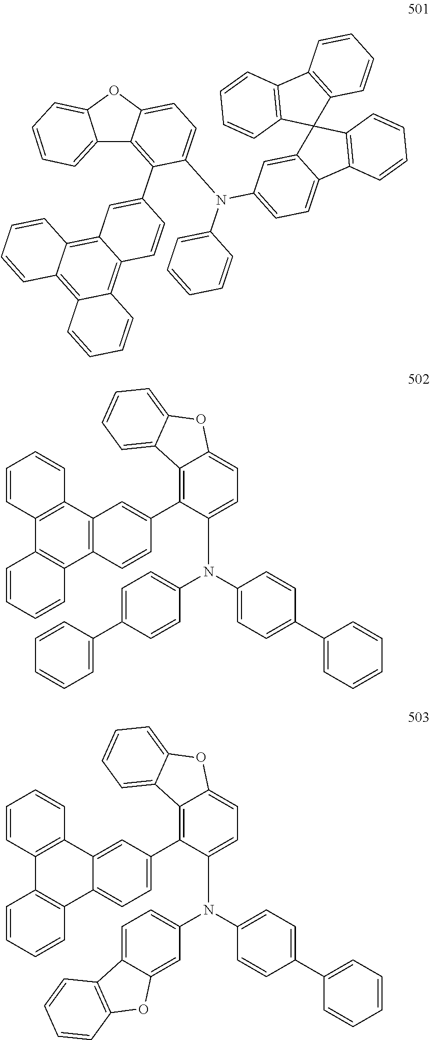

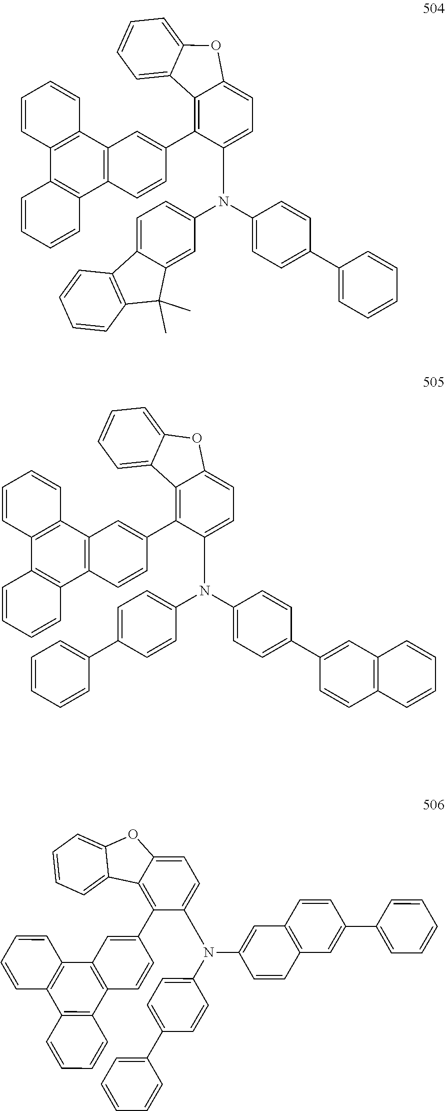 [SMILES: CC1(C)c2ccccc2-c2ccc(N(c3ccc(-c4ccccc4)cc3)c3ccc4oc5ccccc5c4c3-c3ccc4c5ccccc5c5ccccc5c4c3)cc21 ; c1ccc(-c2ccc(N(c3ccc(-c4ccc5ccccc5c4)cc3)c3ccc4oc5ccccc5c4c3-c3ccc4c5ccccc5c5ccccc5c4c3)cc2)cc1 ; c1ccc(-c2ccc(N(c3ccc4cc(-c5ccccc5)ccc4c3)c3ccc4oc5ccccc5c4c3-c3ccc4c5ccccc5c5ccccc5c4c3)cc2)cc1]

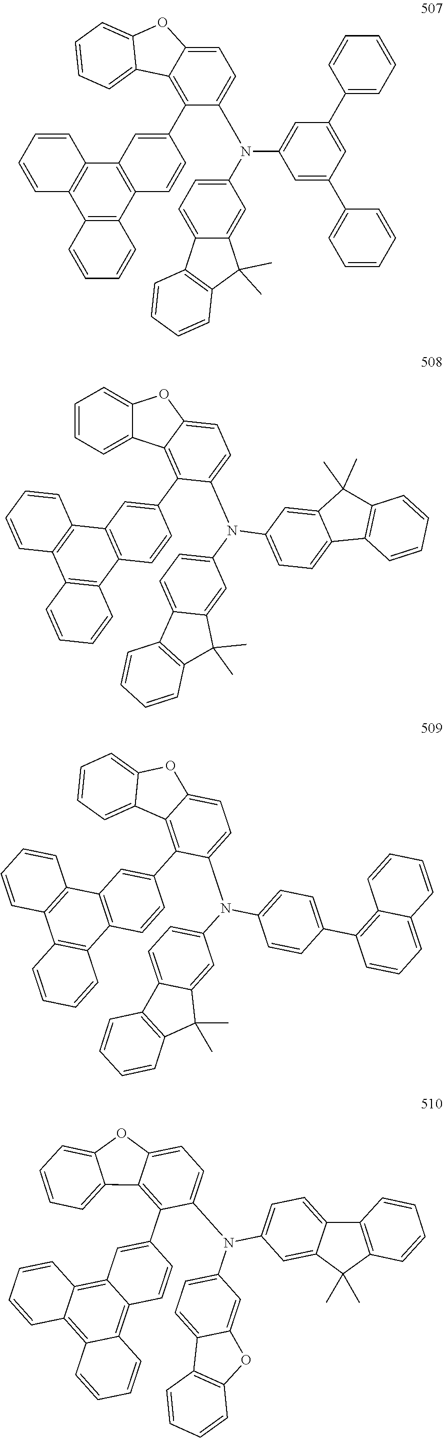 [SMILES: CC1(C)c2ccccc2-c2ccc(N(c3cc(-c4ccccc4)cc(-c4ccccc4)c3)c3ccc4oc5ccccc5c4c3-c3ccc4c5ccccc5c5ccccc5c4c3)cc21 ; CC1(C)c2ccccc2-c2ccc(N(c3ccc4c(c3)C(C)(C)c3ccccc3-4)c3ccc4oc5ccccc5c4c3-c3ccc4c5ccccc5c5ccccc5c4c3)cc21 ; CC1(C)c2ccccc2-c2ccc(N(c3ccc(-c4cccc5ccccc45)cc3)c3ccc4oc5ccccc5c4c3-c3ccc4c5ccccc5c5ccccc5c4c3)cc21 ; CC1(C)c2ccccc2-c2ccc(N(c3ccc4c(c3)oc3ccccc34)c3ccc4oc5ccccc5c4c3-c3ccc4c5ccccc5c5ccccc5c4c3)cc21]

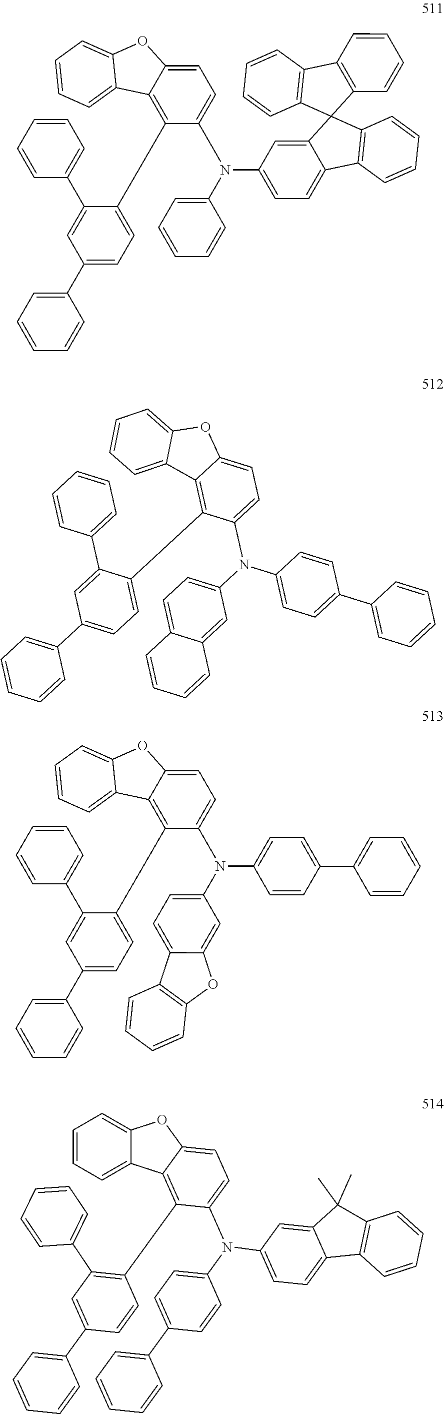 [SMILES: c1ccc(-c2ccc(-c3c(N(c4ccccc4)c4ccc5c(c4)C4(c6ccccc6-c6ccccc64)c4ccccc4-5)ccc4oc5ccccc5c34)c(-c3ccccc3)c2)cc1 ; c1ccc(-c2ccc(N(c3ccc4ccccc4c3)c3ccc4oc5ccccc5c4c3-c3ccc(-c4ccccc4)cc3-c3ccccc3)cc2)cc1 ; c1ccc(-c2ccc(N(c3ccc4c(c3)oc3ccccc34)c3ccc4oc5ccccc5c4c3-c3ccc(-c4ccccc4)cc3-c3ccccc3)cc2)cc1 ; CC1(C)c2ccccc2-c2ccc(N(c3ccc(-c4ccccc4)cc3)c3ccc4oc5ccccc5c4c3-c3ccc(-c4ccccc4)cc3-c3ccccc3)cc21]

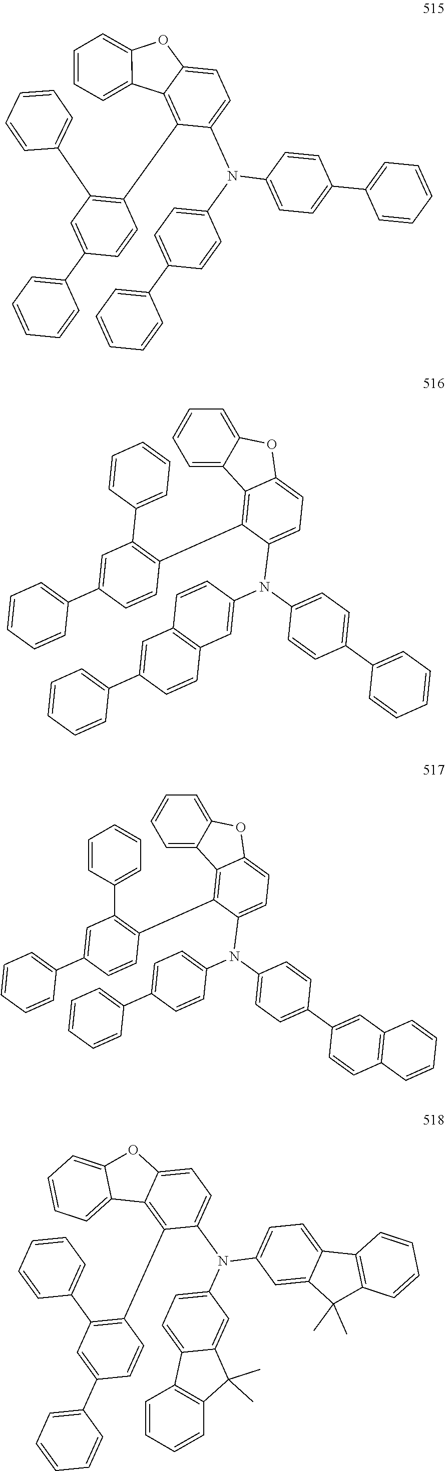 [SMILES: c1ccc(-c2ccc(N(c3ccc(-c4ccccc4)cc3)c3ccc4oc5ccccc5c4c3-c3ccc(-c4ccccc4)cc3-c3ccccc3)cc2)cc1 ; c1ccc(-c2ccc(N(c3ccc4cc(-c5ccccc5)ccc4c3)c3ccc4oc5ccccc5c4c3-c3ccc(-c4ccccc4)cc3-c3ccccc3)cc2)cc1 ; c1ccc(-c2ccc(N(c3ccc(-c4ccc5ccccc5c4)cc3)c3ccc4oc5ccccc5c4c3-c3ccc(-c4ccccc4)cc3-c3ccccc3)cc2)cc1 ; CC1(C)c2ccccc2-c2ccc(N(c3ccc4c(c3)C(C)(C)c3ccccc3-4)c3ccc4oc5ccccc5c4c3-c3ccc(-c4ccccc4)cc3-c3ccccc3)cc21]

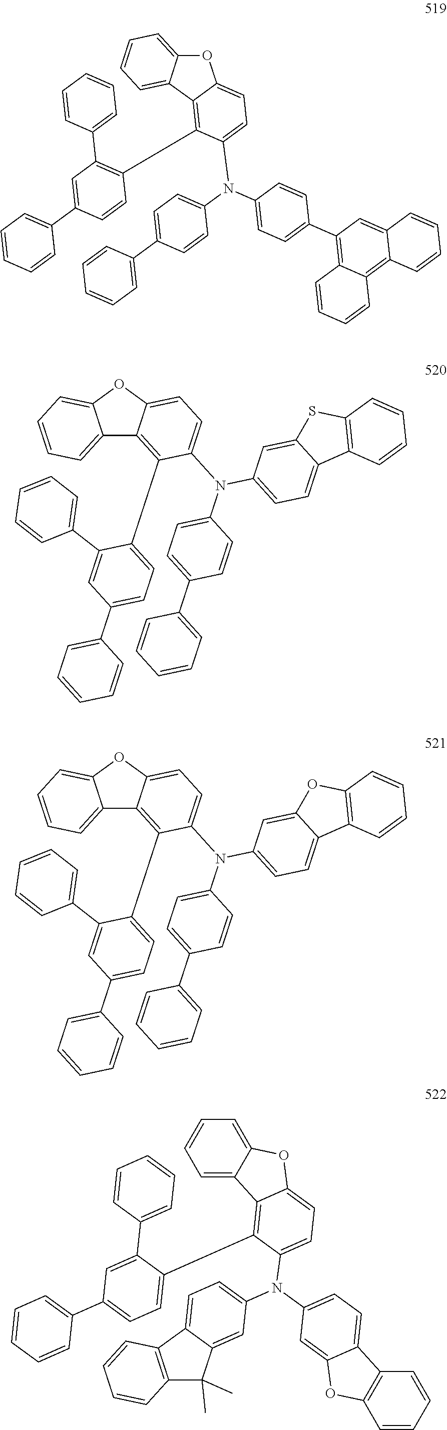 [SMILES: c1ccc(-c2ccc(N(c3ccc(-c4cc5ccccc5c5ccccc45)cc3)c3ccc4oc5ccccc5c4c3-c3ccc(-c4ccccc4)cc3-c3ccccc3)cc2)cc1 ; c1ccc(-c2ccc(N(c3ccc4c(c3)sc3ccccc34)c3ccc4oc5ccccc5c4c3-c3ccc(-c4ccccc4)cc3-c3ccccc3)cc2)cc1 ; c1ccc(-c2ccc(N(c3ccc4c(c3)oc3ccccc34)c3ccc4oc5ccccc5c4c3-c3ccc(-c4ccccc4)cc3-c3ccccc3)cc2)cc1 ; CC1(C)c2ccccc2-c2ccc(N(c3ccc4c(c3)oc3ccccc34)c3ccc4oc5ccccc5c4c3-c3ccc(-c4ccccc4)cc3-c3ccccc3)cc21]

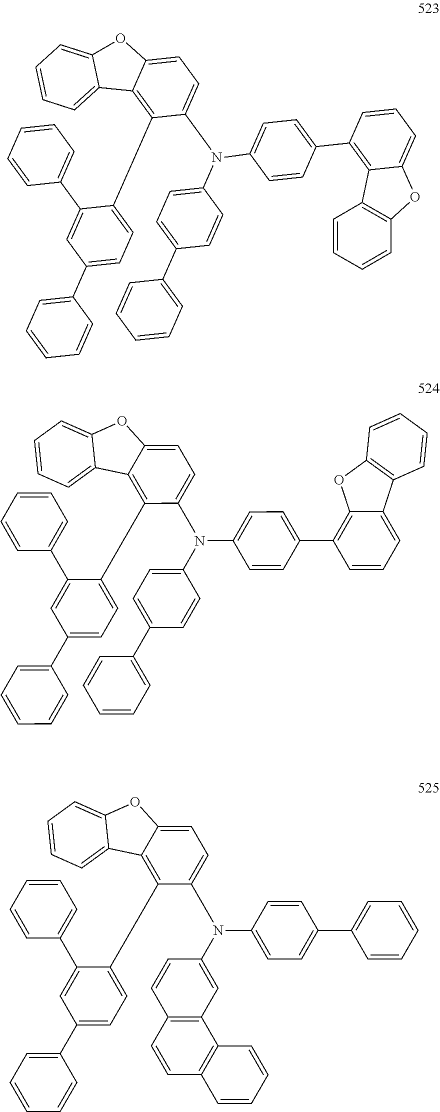 [SMILES: c1ccc(-c2ccc(N(c3ccc(-c4cccc5oc6ccccc6c45)cc3)c3ccc4oc5ccccc5c4c3-c3ccc(-c4ccccc4)cc3-c3ccccc3)cc2)cc1 ; c1ccc(-c2ccc(N(c3ccc(-c4cccc5c4oc4ccccc45)cc3)c3ccc4oc5ccccc5c4c3-c3ccc(-c4ccccc4)cc3-c3ccccc3)cc2)cc1 ; c1ccc(-c2ccc(N(c3ccc4ccc5ccccc5c4c3)c3ccc4oc5ccccc5c4c3-c3ccc(-c4ccccc4)cc3-c3ccccc3)cc2)cc1]

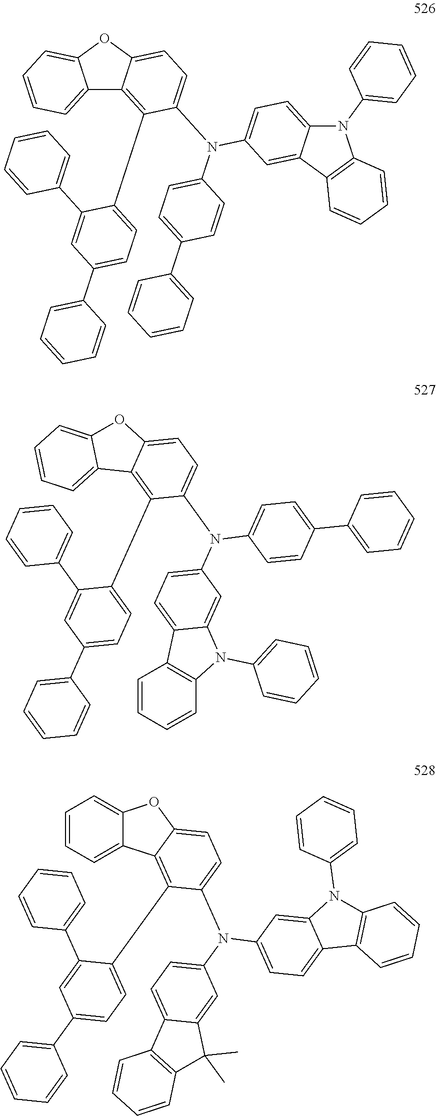 [SMILES: c1ccc(-c2ccc(N(c3ccc4c(c3)c3ccccc3n4-c3ccccc3)c3ccc4oc5ccccc5c4c3-c3ccc(-c4ccccc4)cc3-c3ccccc3)cc2)cc1 ; c1ccc(-c2ccc(N(c3ccc4c5ccccc5n(-c5ccccc5)c4c3)c3ccc4oc5ccccc5c4c3-c3ccc(-c4ccccc4)cc3-c3ccccc3)cc2)cc1 ; CC1(C)c2ccccc2-c2ccc(N(c3ccc4c5ccccc5n(-c5ccccc5)c4c3)c3ccc4oc5ccccc5c4c3-c3ccc(-c4ccccc4)cc3-c3ccccc3)cc21]

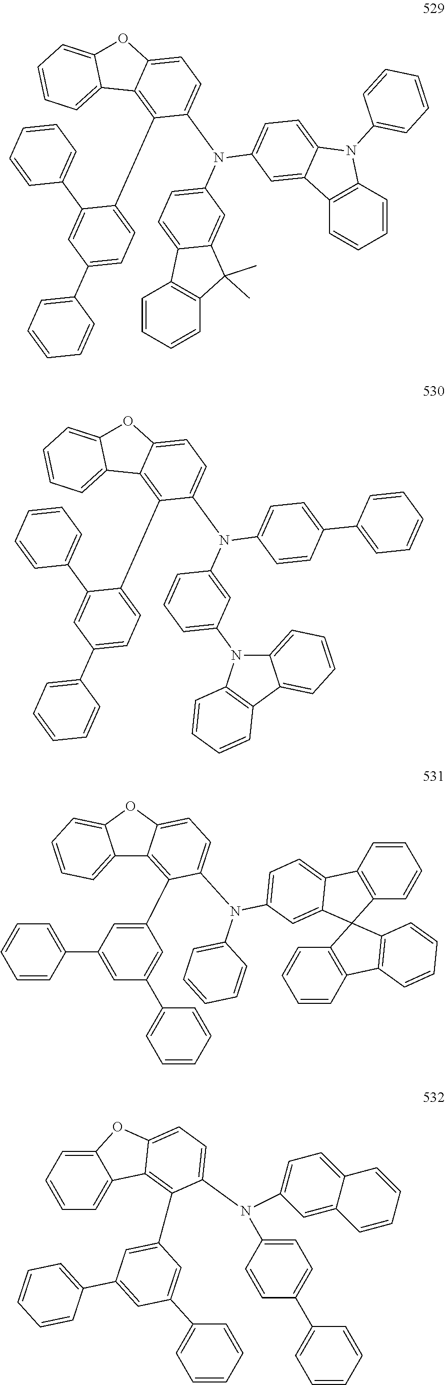 [SMILES: CC1(C)c2ccccc2-c2ccc(N(c3ccc4c(c3)c3ccccc3n4-c3ccccc3)c3ccc4oc5ccccc5c4c3-c3ccc(-c4ccccc4)cc3-c3ccccc3)cc21 ; c1ccc(-c2ccc(N(c3cccc(-n4c5ccccc5c5ccccc54)c3)c3ccc4oc5ccccc5c4c3-c3ccc(-c4ccccc4)cc3-c3ccccc3)cc2)cc1 ; c1ccc(-c2cc(-c3ccccc3)cc(-c3c(N(c4ccccc4)c4ccc5c(c4)C4(c6ccccc6-c6ccccc64)c4ccccc4-5)ccc4oc5ccccc5c34)c2)cc1 ; c1ccc(-c2ccc(N(c3ccc4ccccc4c3)c3ccc4oc5ccccc5c4c3-c3cc(-c4ccccc4)cc(-c4ccccc4)c3)cc2)cc1]

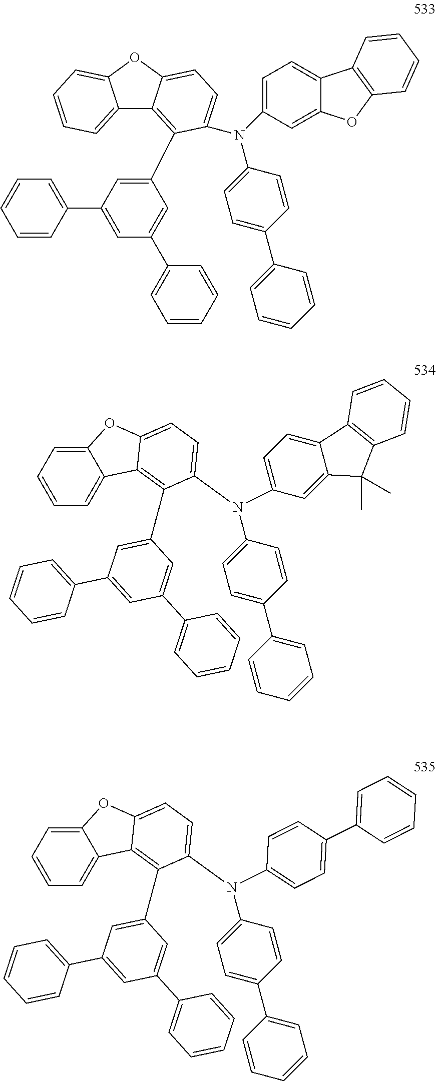 [SMILES: c1ccc(-c2ccc(N(c3ccc4c(c3)oc3ccccc34)c3ccc4oc5ccccc5c4c3-c3cc(-c4ccccc4)cc(-c4ccccc4)c3)cc2)cc1 ; CC1(C)c2ccccc2-c2ccc(N(c3ccc(-c4ccccc4)cc3)c3ccc4oc5ccccc5c4c3-c3cc(-c4ccccc4)cc(-c4ccccc4)c3)cc21 ; c1ccc(-c2ccc(N(c3ccc(-c4ccccc4)cc3)c3ccc4oc5ccccc5c4c3-c3cc(-c4ccccc4)cc(-c4ccccc4)c3)cc2)cc1]

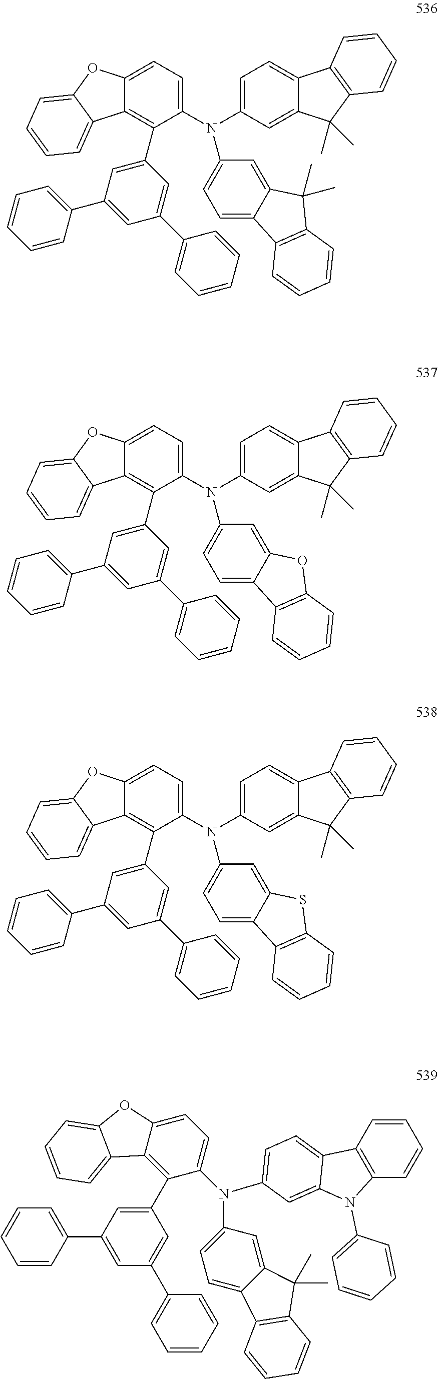 [SMILES: CC1(C)c2ccccc2-c2ccc(N(c3ccc4c(c3)C(C)(C)c3ccccc3-4)c3ccc4oc5ccccc5c4c3-c3cc(-c4ccccc4)cc(-c4ccccc4)c3)cc21 ; CC1(C)c2ccccc2-c2ccc(N(c3ccc4c(c3)oc3ccccc34)c3ccc4oc5ccccc5c4c3-c3cc(-c4ccccc4)cc(-c4ccccc4)c3)cc21 ; CC1(C)c2ccccc2-c2ccc(N(c3ccc4c(c3)sc3ccccc34)c3ccc4oc5ccccc5c4c3-c3cc(-c4ccccc4)cc(-c4ccccc4)c3)cc21 ; CC1(C)c2ccccc2-c2ccc(N(c3ccc4c5ccccc5n(-c5ccccc5)c4c3)c3ccc4oc5ccccc5c4c3-c3cc(-c4ccccc4)cc(-c4ccccc4)c3)cc21]

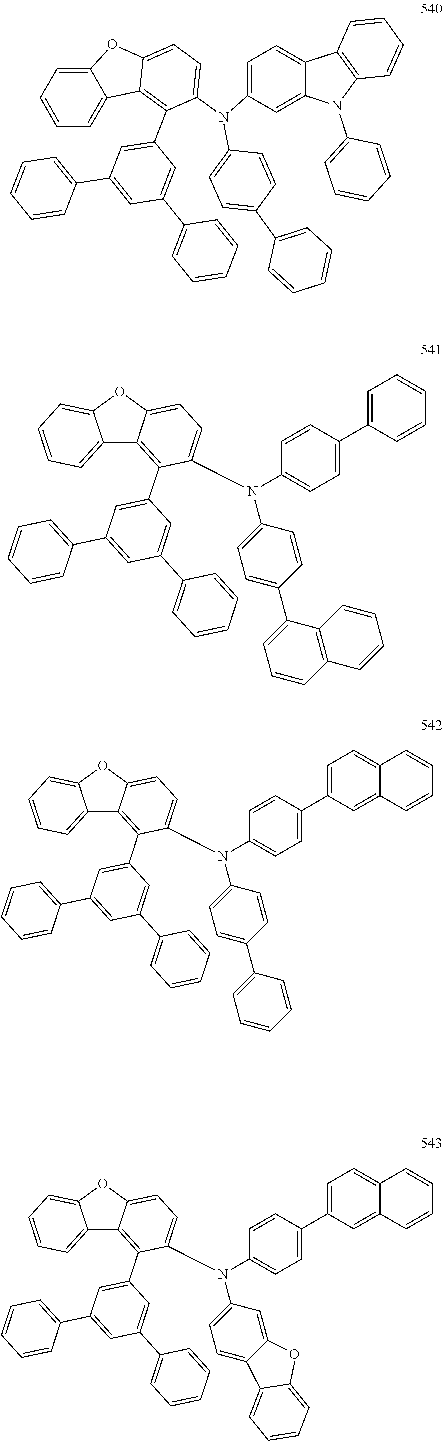 [SMILES: c1ccc(-c2ccc(N(c3ccc4c5ccccc5n(-c5ccccc5)c4c3)c3ccc4oc5ccccc5c4c3-c3cc(-c4ccccc4)cc(-c4ccccc4)c3)cc2)cc1 ; c1ccc(-c2ccc(N(c3ccc(-c4cccc5ccccc45)cc3)c3ccc4oc5ccccc5c4c3-c3cc(-c4ccccc4)cc(-c4ccccc4)c3)cc2)cc1 ; c1ccc(-c2ccc(N(c3ccc(-c4ccc5ccccc5c4)cc3)c3ccc4oc5ccccc5c4c3-c3cc(-c4ccccc4)cc(-c4ccccc4)c3)cc2)cc1 ; c1ccc(-c2cc(-c3ccccc3)cc(-c3c(N(c4ccc(-c5ccc6ccccc6c5)cc4)c4ccc5c(c4)oc4ccccc45)ccc4oc5ccccc5c34)c2)cc1]

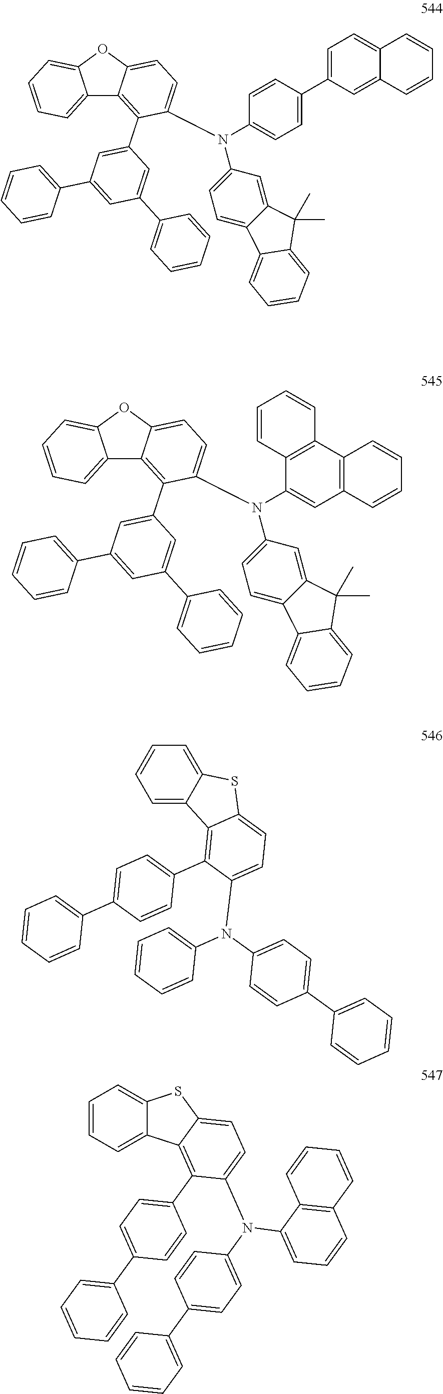 [SMILES: CC1(C)c2ccccc2-c2ccc(N(c3ccc(-c4ccc5ccccc5c4)cc3)c3ccc4oc5ccccc5c4c3-c3cc(-c4ccccc4)cc(-c4ccccc4)c3)cc21 ; CC1(C)c2ccccc2-c2ccc(N(c3ccc4oc5ccccc5c4c3-c3cc(-c4ccccc4)cc(-c4ccccc4)c3)c3cc4ccccc4c4ccccc34)cc21 ; c1ccc(-c2ccc(-c3c(N(c4ccccc4)c4ccc(-c5ccccc5)cc4)ccc4sc5ccccc5c34)cc2)cc1 ; c1ccc(-c2ccc(-c3c(N(c4ccc(-c5ccccc5)cc4)c4cccc5ccccc45)ccc4sc5ccccc5c34)cc2)cc1]

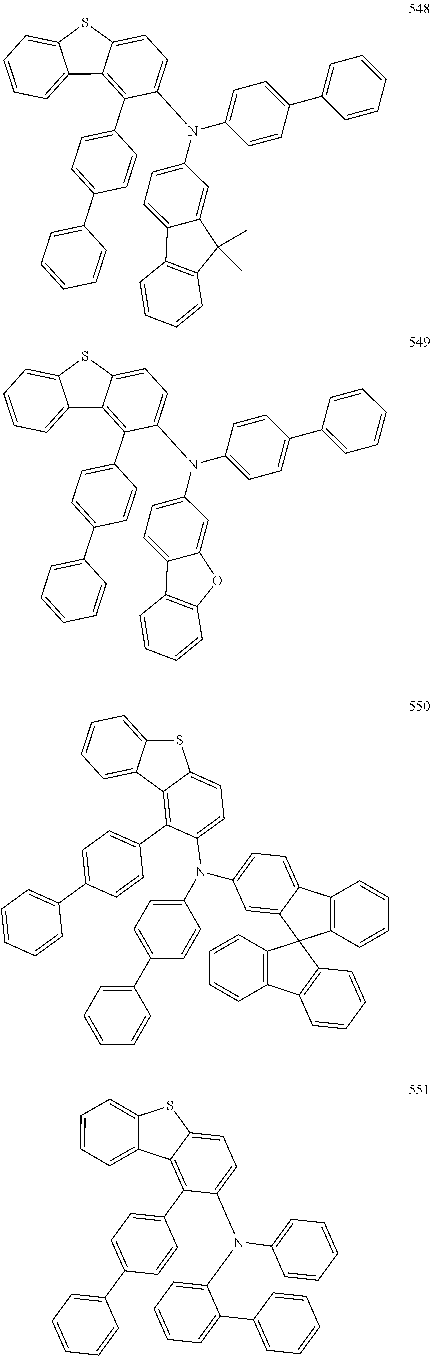 [SMILES: CC1(C)c2ccccc2-c2ccc(N(c3ccc(-c4ccccc4)cc3)c3ccc4sc5ccccc5c4c3-c3ccc(-c4ccccc4)cc3)cc21 ; c1ccc(-c2ccc(-c3c(N(c4ccc(-c5ccccc5)cc4)c4ccc5c(c4)oc4ccccc45)ccc4sc5ccccc5c34)cc2)cc1 ; c1ccc(-c2ccc(-c3c(N(c4ccc(-c5ccccc5)cc4)c4ccc5c(c4)C4(c6ccccc6-c6ccccc64)c4ccccc4-5)ccc4sc5ccccc5c34)cc2)cc1 ; c1ccc(-c2ccc(-c3c(N(c4ccccc4)c4ccccc4-c4ccccc4)ccc4sc5ccccc5c34)cc2)cc1]

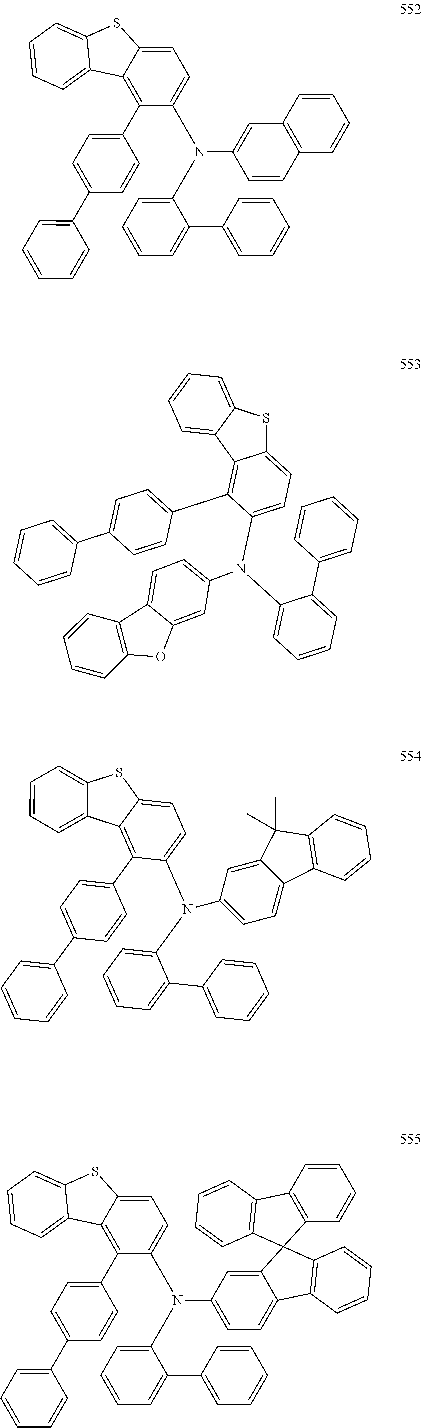 [SMILES: c1ccc(-c2ccc(-c3c(N(c4ccc5ccccc5c4)c4ccccc4-c4ccccc4)ccc4sc5ccccc5c34)cc2)cc1 ; c1ccc(-c2ccc(-c3c(N(c4ccc5c(c4)oc4ccccc45)c4ccccc4-c4ccccc4)ccc4sc5ccccc5c34)cc2)cc1 ; CC1(C)c2ccccc2-c2ccc(N(c3ccccc3-c3ccccc3)c3ccc4sc5ccccc5c4c3-c3ccc(-c4ccccc4)cc3)cc21 ; c1ccc(-c2ccc(-c3c(N(c4ccc5c(c4)C4(c6ccccc6-c6ccccc64)c4ccccc4-5)c4ccccc4-c4ccccc4)ccc4sc5ccccc5c34)cc2)cc1]

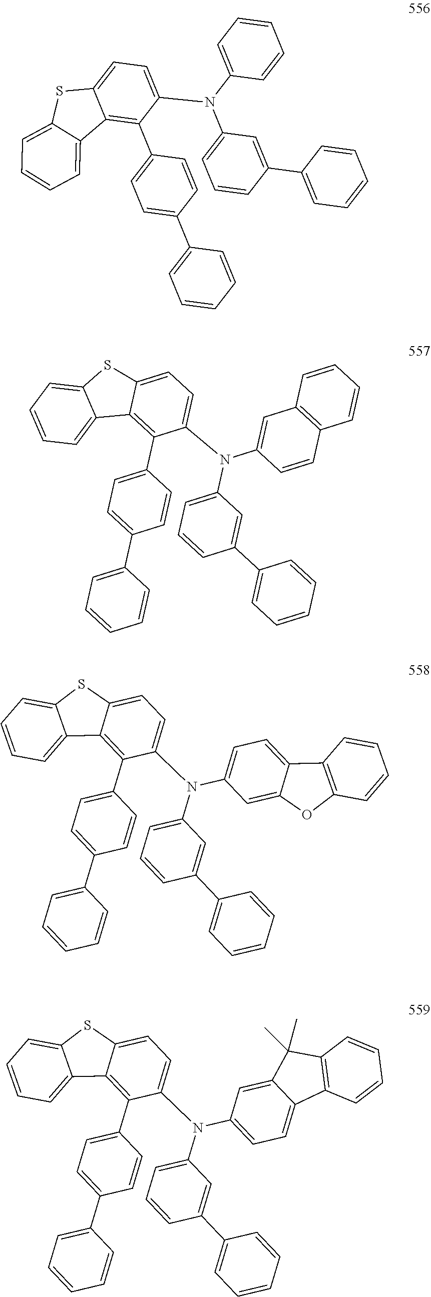 [SMILES: c1ccc(-c2ccc(-c3c(N(c4ccccc4)c4cccc(-c5ccccc5)c4)ccc4sc5ccccc5c34)cc2)cc1 ; c1ccc(-c2ccc(-c3c(N(c4cccc(-c5ccccc5)c4)c4ccc5ccccc5c4)ccc4sc5ccccc5c34)cc2)cc1 ; c1ccc(-c2ccc(-c3c(N(c4cccc(-c5ccccc5)c4)c4ccc5c(c4)oc4ccccc45)ccc4sc5ccccc5c34)cc2)cc1 ; CC1(C)c2ccccc2-c2ccc(N(c3cccc(-c4ccccc4)c3)c3ccc4sc5ccccc5c4c3-c3ccc(-c4ccccc4)cc3)cc21]

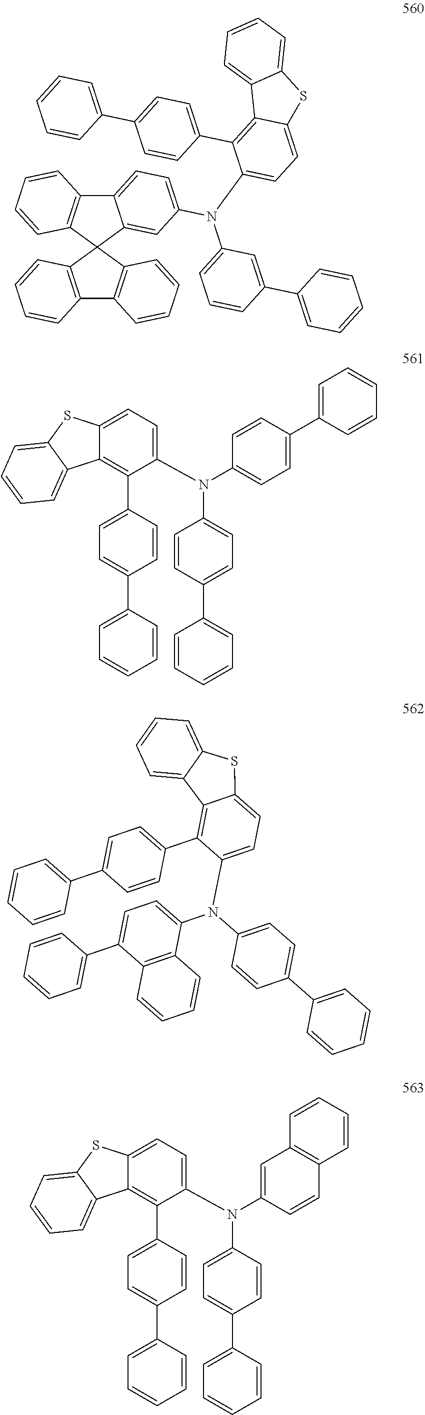

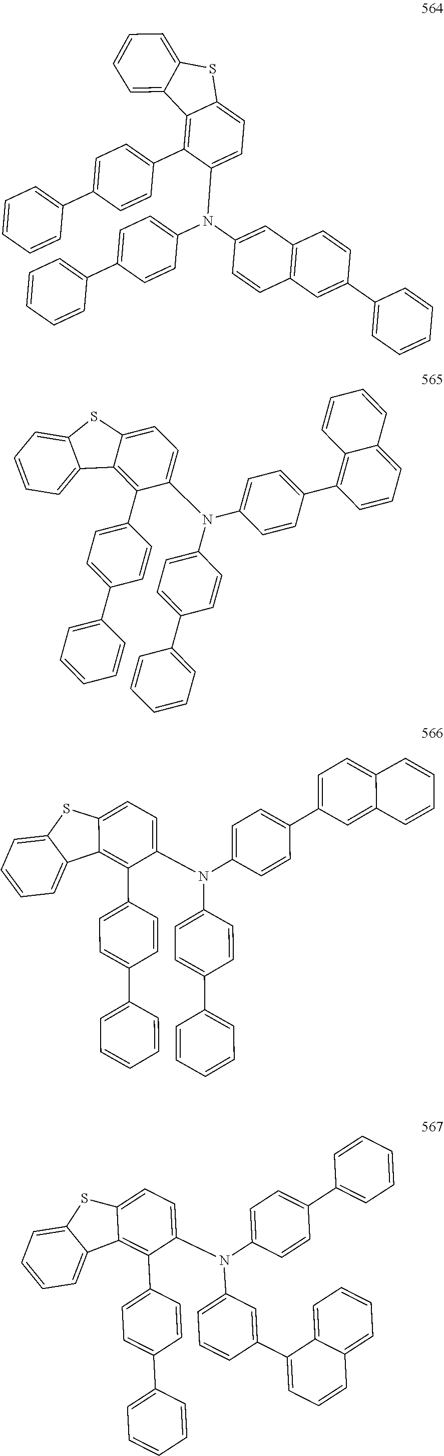 [SMILES: c1ccc(-c2ccc(-c3c(N(c4ccc(-c5ccccc5)cc4)c4ccc5cc(-c6ccccc6)ccc5c4)ccc4sc5ccccc5c34)cc2)cc1 ; c1ccc(-c2ccc(-c3c(N(c4ccc(-c5ccccc5)cc4)c4ccc(-c5cccc6ccccc56)cc4)ccc4sc5ccccc5c34)cc2)cc1 ; c1ccc(-c2ccc(-c3c(N(c4ccc(-c5ccccc5)cc4)c4ccc(-c5ccc6ccccc6c5)cc4)ccc4sc5ccccc5c34)cc2)cc1 ; c1ccc(-c2ccc(-c3c(N(c4ccc(-c5ccccc5)cc4)c4cccc(-c5cccc6ccccc56)c4)ccc4sc5ccccc5c34)cc2)cc1]

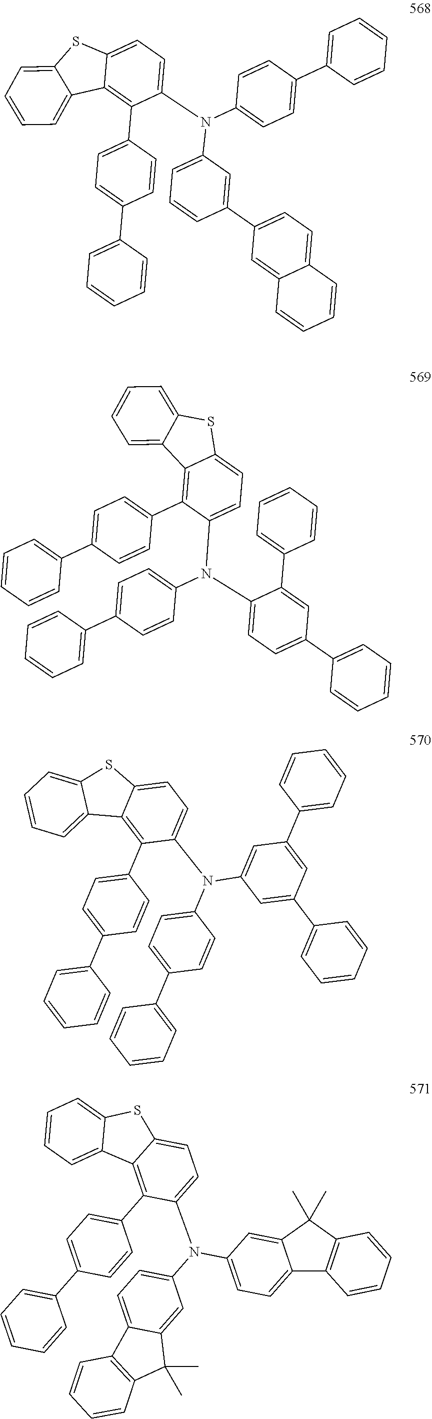

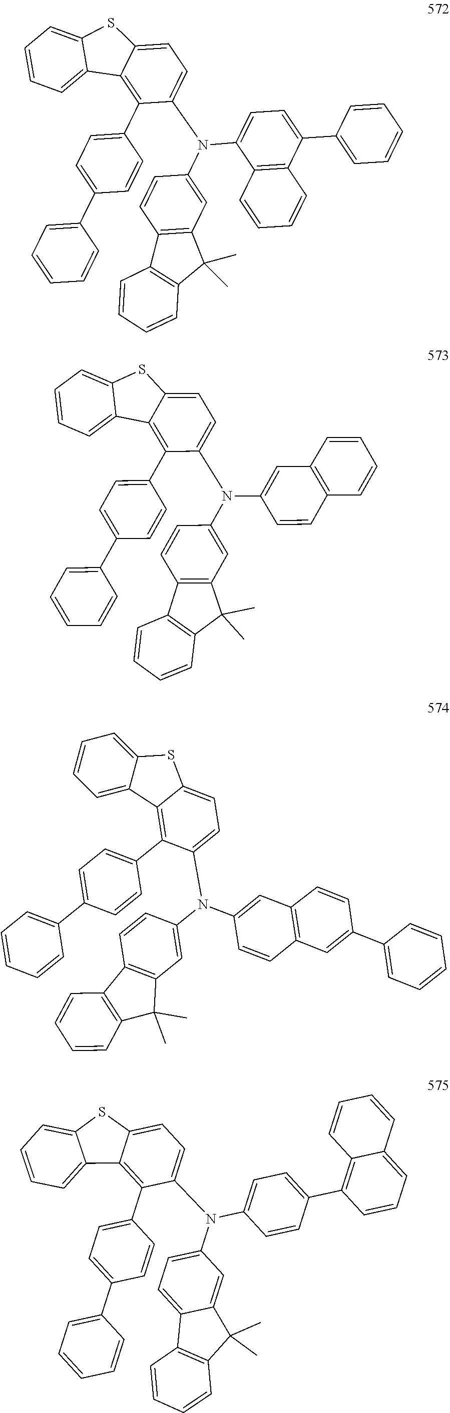

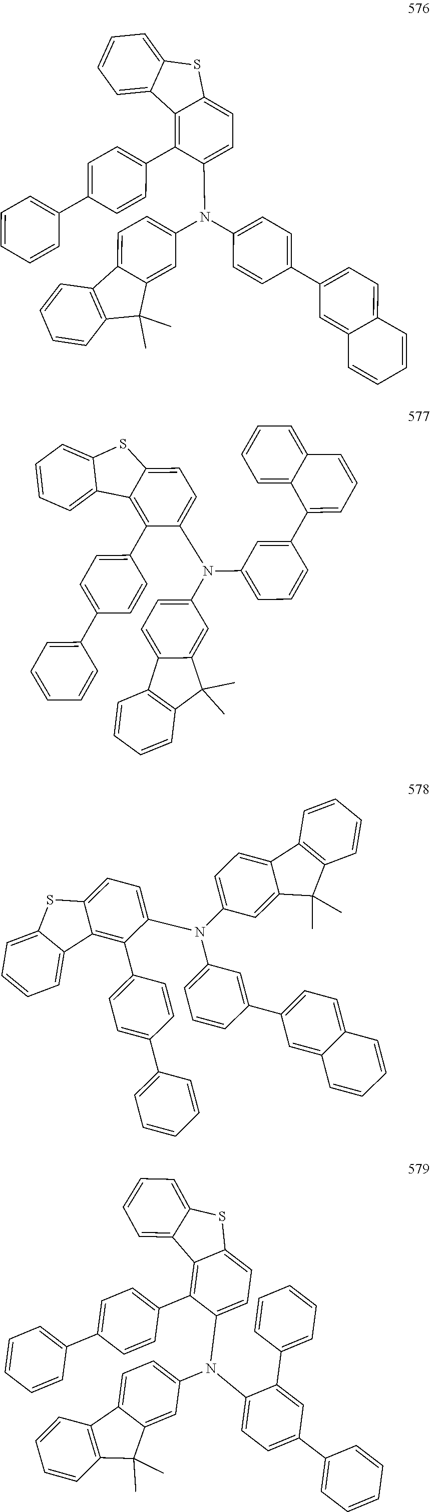

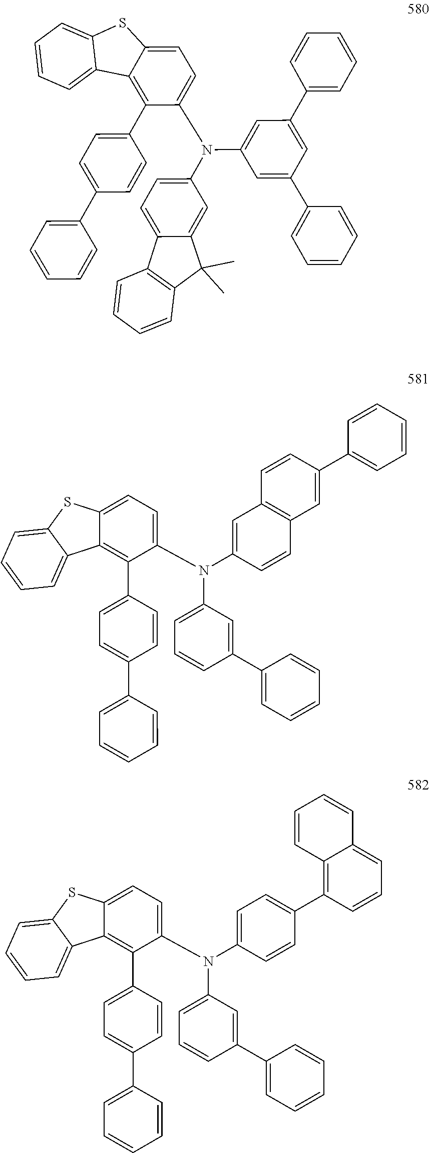 [SMILES: CC1(C)c2ccccc2-c2ccc(N(c3cc(-c4ccccc4)cc(-c4ccccc4)c3)c3ccc4sc5ccccc5c4c3-c3ccc(-c4ccccc4)cc3)cc21 ; c1ccc(-c2ccc(-c3c(N(c4cccc(-c5ccccc5)c4)c4ccc5cc(-c6ccccc6)ccc5c4)ccc4sc5ccccc5c34)cc2)cc1 ; c1ccc(-c2ccc(-c3c(N(c4ccc(-c5cccc6ccccc56)cc4)c4cccc(-c5ccccc5)c4)ccc4sc5ccccc5c34)cc2)cc1]

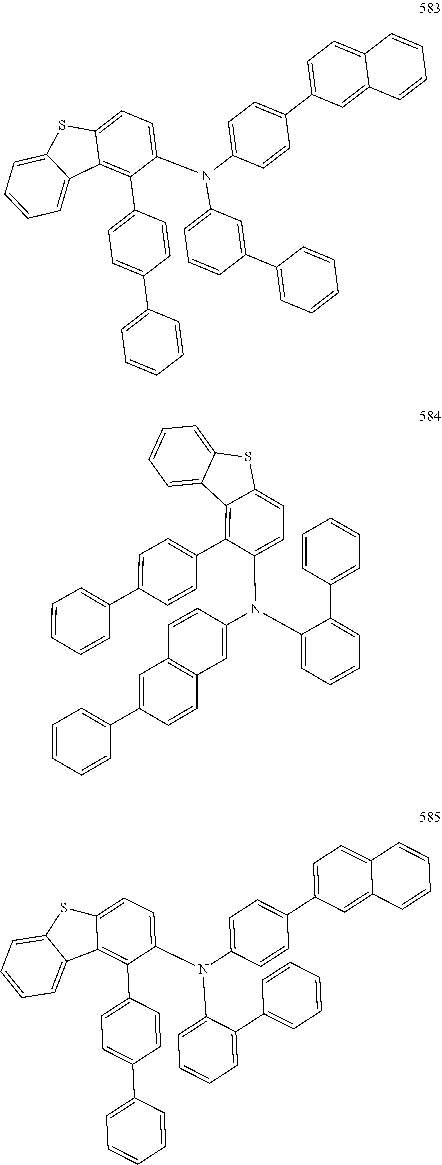 [SMILES: c1ccc(-c2ccc(-c3c(N(c4ccc(-c5ccc6ccccc6c5)cc4)c4cccc(-c5ccccc5)c4)ccc4sc5ccccc5c34)cc2)cc1 ; c1ccc(-c2ccc(-c3c(N(c4ccc5cc(-c6ccccc6)ccc5c4)c4ccccc4-c4ccccc4)ccc4sc5ccccc5c34)cc2)cc1 ; c1ccc(-c2ccc(-c3c(N(c4ccc(-c5ccc6ccccc6c5)cc4)c4ccccc4-c4ccccc4)ccc4sc5ccccc5c34)cc2)cc1]

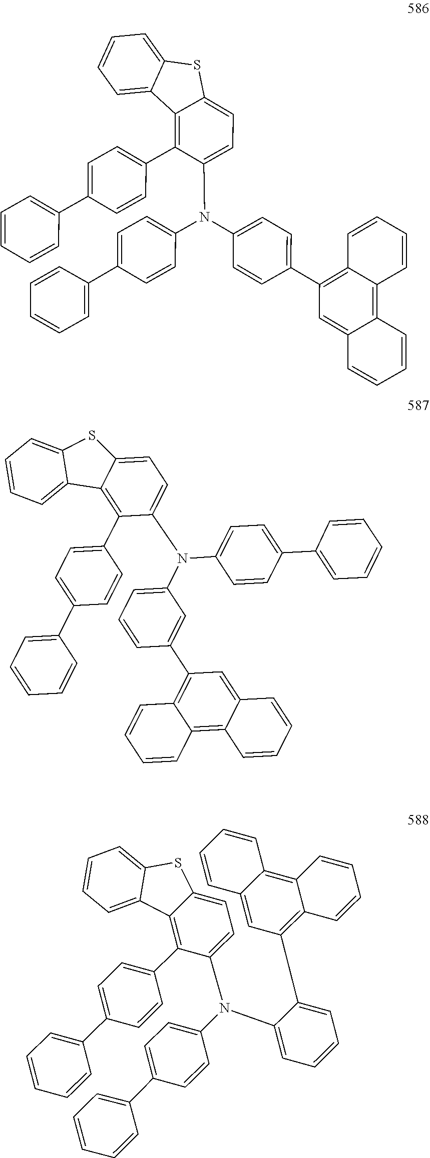 [SMILES: c1ccc(-c2ccc(-c3c(N(c4ccc(-c5ccccc5)cc4)c4ccc(-c5cc6ccccc6c6ccccc56)cc4)ccc4sc5ccccc5c34)cc2)cc1 ; c1ccc(-c2ccc(-c3c(N(c4ccc(-c5ccccc5)cc4)c4cccc(-c5cc6ccccc6c6ccccc56)c4)ccc4sc5ccccc5c34)cc2)cc1 ; c1ccc(-c2ccc(-c3c(N(c4ccc(-c5ccccc5)cc4)c4ccccc4-c4cc5ccccc5c5ccccc45)ccc4sc5ccccc5c34)cc2)cc1]

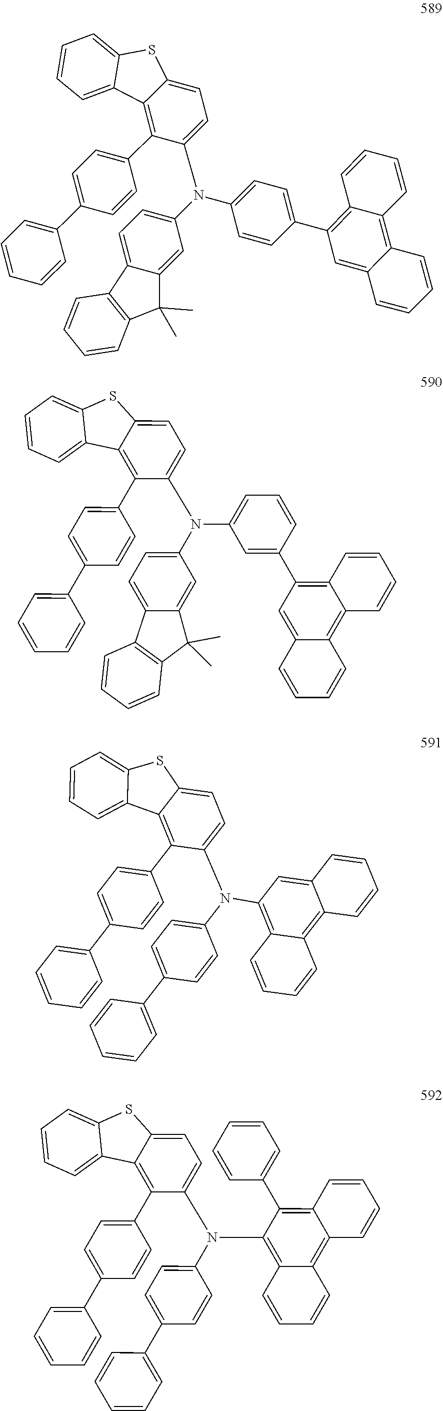

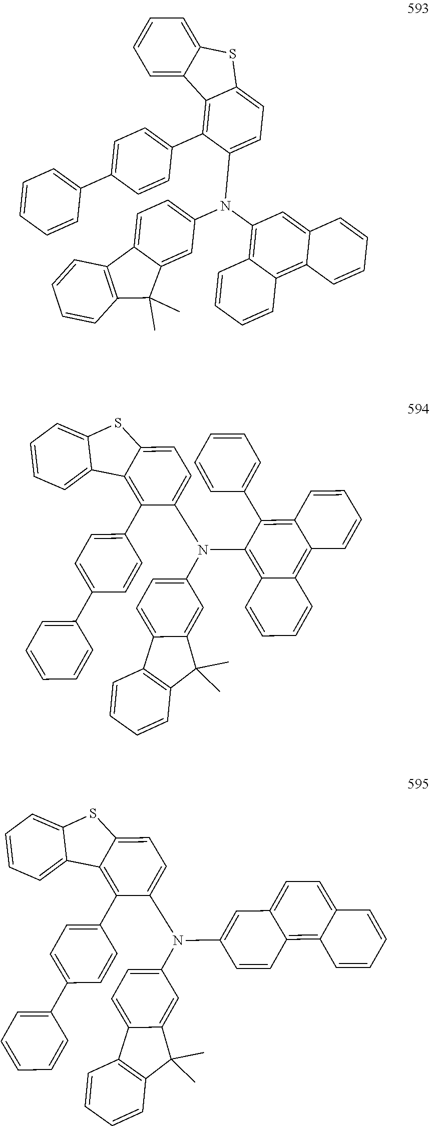 [SMILES: CC1(C)c2ccccc2-c2ccc(N(c3ccc4sc5ccccc5c4c3-c3ccc(-c4ccccc4)cc3)c3cc4ccccc4c4ccccc34)cc21 ; CC1(C)c2ccccc2-c2ccc(N(c3ccc4sc5ccccc5c4c3-c3ccc(-c4ccccc4)cc3)c3c(-c4ccccc4)c4ccccc4c4ccccc34)cc21 ; CC1(C)c2ccccc2-c2ccc(N(c3ccc4c(ccc5ccccc54)c3)c3ccc4sc5ccccc5c4c3-c3ccc(-c4ccccc4)cc3)cc21]

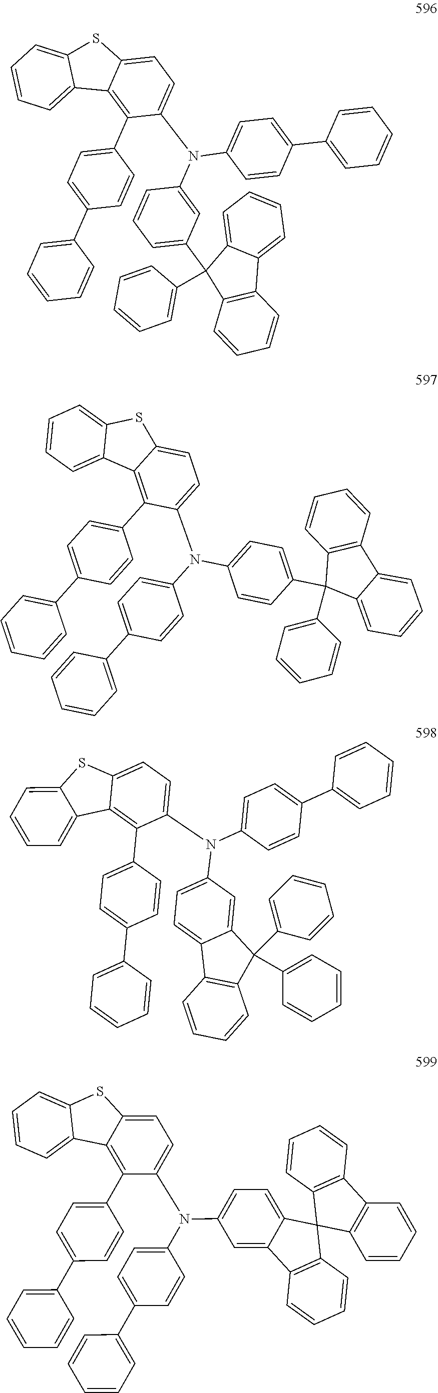 [SMILES: c1ccc(-c2ccc(-c3c(N(c4ccc(-c5ccccc5)cc4)c4cccc(C5(c6ccccc6)c6ccccc6-c6ccccc65)c4)ccc4sc5ccccc5c34)cc2)cc1 ; c1ccc(-c2ccc(-c3c(N(c4ccc(-c5ccccc5)cc4)c4ccc(C5(c6ccccc6)c6ccccc6-c6ccccc65)cc4)ccc4sc5ccccc5c34)cc2)cc1 ; c1ccc(-c2ccc(-c3c(N(c4ccc(-c5ccccc5)cc4)c4ccc5c(c4)C(c4ccccc4)(c4ccccc4)c4ccccc4-5)ccc4sc5ccccc5c34)cc2)cc1 ; c1ccc(-c2ccc(-c3c(N(c4ccc(-c5ccccc5)cc4)c4ccc5c(c4)-c4ccccc4C54c5ccccc5-c5ccccc54)ccc4sc5ccccc5c34)cc2)cc1]

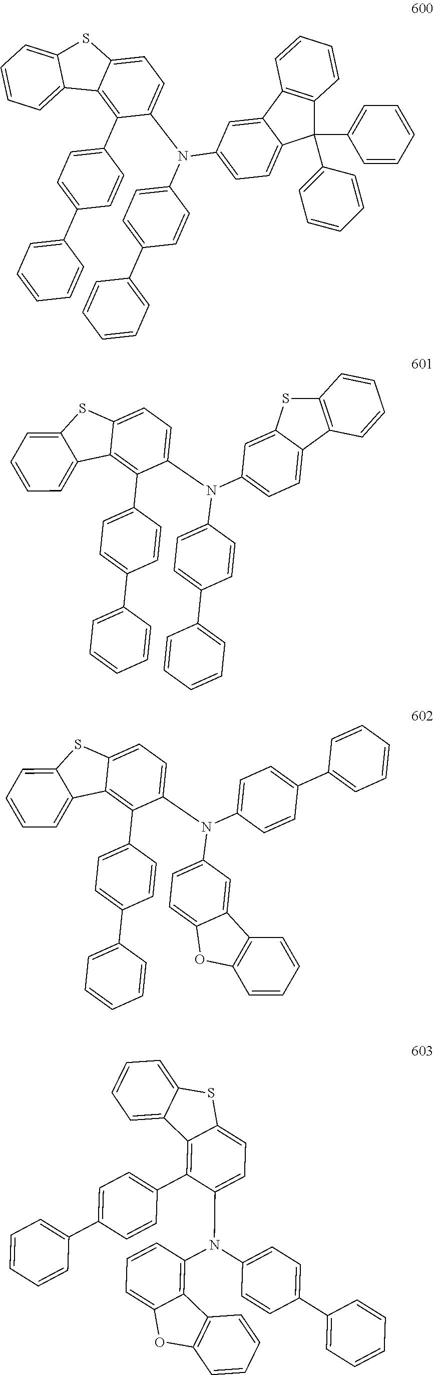 [SMILES: c1ccc(-c2ccc(-c3c(N(c4ccc(-c5ccccc5)cc4)c4ccc5c(c4)-c4ccccc4C5(c4ccccc4)c4ccccc4)ccc4sc5ccccc5c34)cc2)cc1 ; c1ccc(-c2ccc(-c3c(N(c4ccc(-c5ccccc5)cc4)c4ccc5c(c4)sc4ccccc45)ccc4sc5ccccc5c34)cc2)cc1 ; c1ccc(-c2ccc(-c3c(N(c4ccc(-c5ccccc5)cc4)c4ccc5oc6ccccc6c5c4)ccc4sc5ccccc5c34)cc2)cc1 ; c1ccc(-c2ccc(-c3c(N(c4ccc(-c5ccccc5)cc4)c4cccc5oc6ccccc6c45)ccc4sc5ccccc5c34)cc2)cc1]

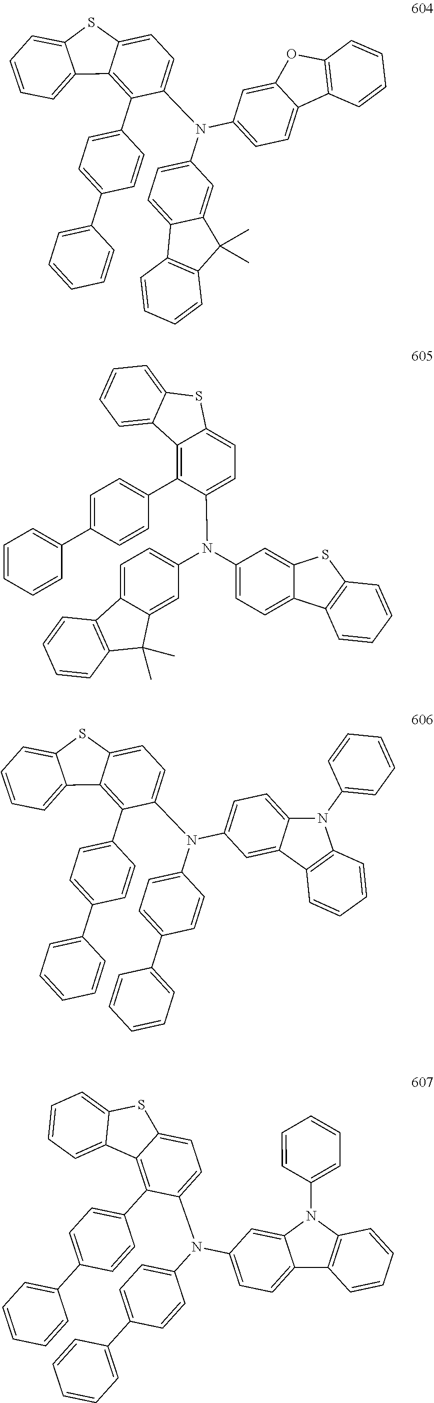 [SMILES: CC1(C)c2ccccc2-c2ccc(N(c3ccc4c(c3)oc3ccccc34)c3ccc4sc5ccccc5c4c3-c3ccc(-c4ccccc4)cc3)cc21 ; CC1(C)c2ccccc2-c2ccc(N(c3ccc4c(c3)sc3ccccc34)c3ccc4sc5ccccc5c4c3-c3ccc(-c4ccccc4)cc3)cc21 ; c1ccc(-c2ccc(-c3c(N(c4ccc(-c5ccccc5)cc4)c4ccc5c(c4)c4ccccc4n5-c4ccccc4)ccc4sc5ccccc5c34)cc2)cc1 ; c1ccc(-c2ccc(-c3c(N(c4ccc(-c5ccccc5)cc4)c4ccc5c6ccccc6n(-c6ccccc6)c5c4)ccc4sc5ccccc5c34)cc2)cc1]

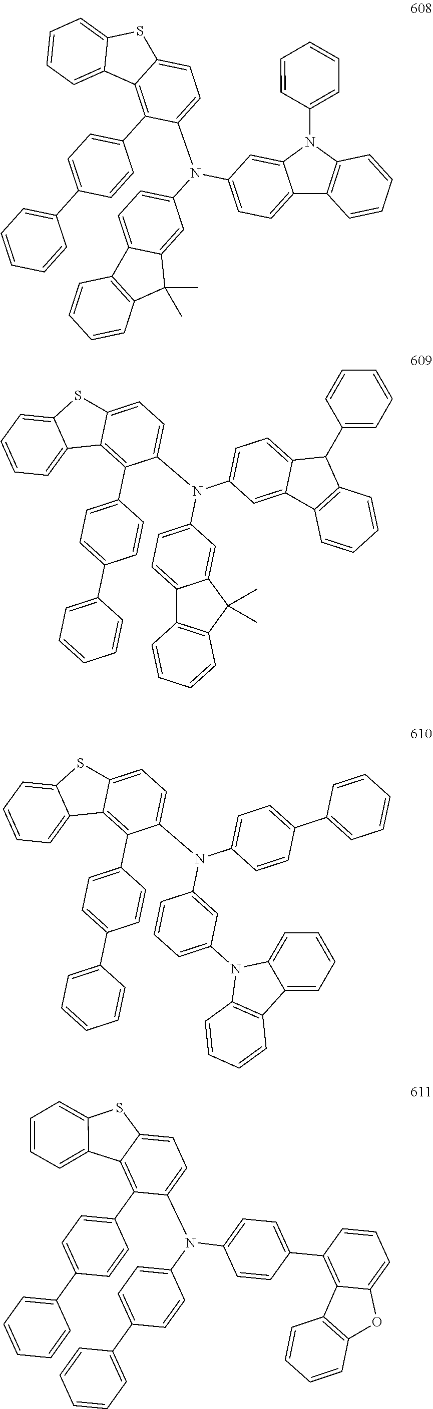 [SMILES: CC1(C)c2ccccc2-c2ccc(N(c3ccc4c5ccccc5n(-c5ccccc5)c4c3)c3ccc4sc5ccccc5c4c3-c3ccc(-c4ccccc4)cc3)cc21 ; CC1(C)c2ccccc2-c2ccc(N(c3ccc4c(c3)-c3ccccc3C4c3ccccc3)c3ccc4sc5ccccc5c4c3-c3ccc(-c4ccccc4)cc3)cc21 ; c1ccc(-c2ccc(-c3c(N(c4ccc(-c5ccccc5)cc4)c4cccc(-n5c6ccccc6c6ccccc65)c4)ccc4sc5ccccc5c34)cc2)cc1 ; c1ccc(-c2ccc(-c3c(N(c4ccc(-c5ccccc5)cc4)c4ccc(-c5cccc6oc7ccccc7c56)cc4)ccc4sc5ccccc5c34)cc2)cc1]

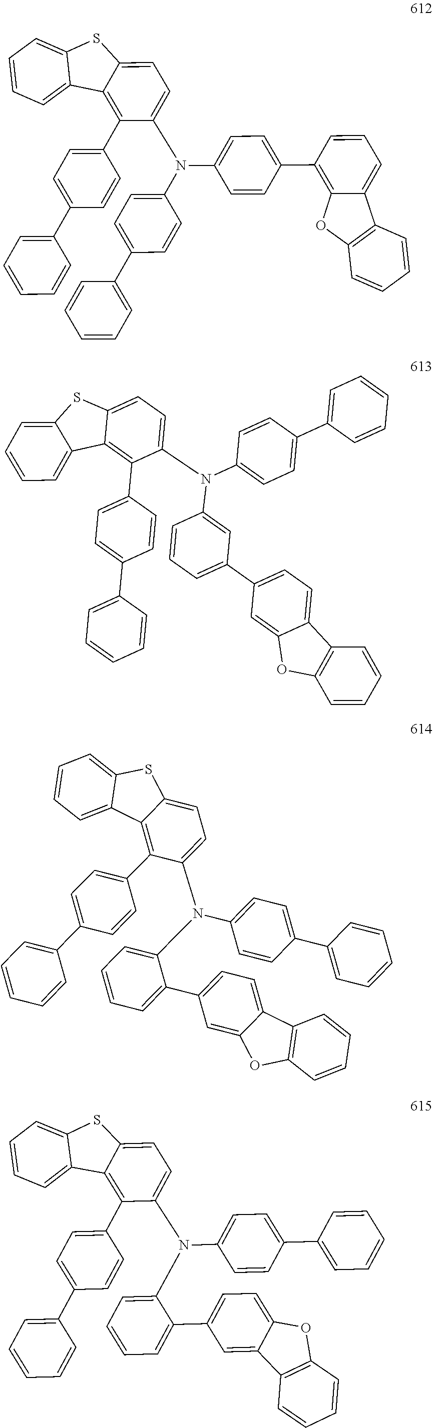 [SMILES: c1ccc(-c2ccc(-c3c(N(c4ccc(-c5ccccc5)cc4)c4ccc(-c5cccc6c5oc5ccccc56)cc4)ccc4sc5ccccc5c34)cc2)cc1 ; c1ccc(-c2ccc(-c3c(N(c4ccc(-c5ccccc5)cc4)c4cccc(-c5ccc6c(c5)oc5ccccc56)c4)ccc4sc5ccccc5c34)cc2)cc1 ; c1ccc(-c2ccc(-c3c(N(c4ccc(-c5ccccc5)cc4)c4ccccc4-c4ccc5c(c4)oc4ccccc45)ccc4sc5ccccc5c34)cc2)cc1 ; c1ccc(-c2ccc(-c3c(N(c4ccc(-c5ccccc5)cc4)c4ccccc4-c4ccc5oc6ccccc6c5c4)ccc4sc5ccccc5c34)cc2)cc1]

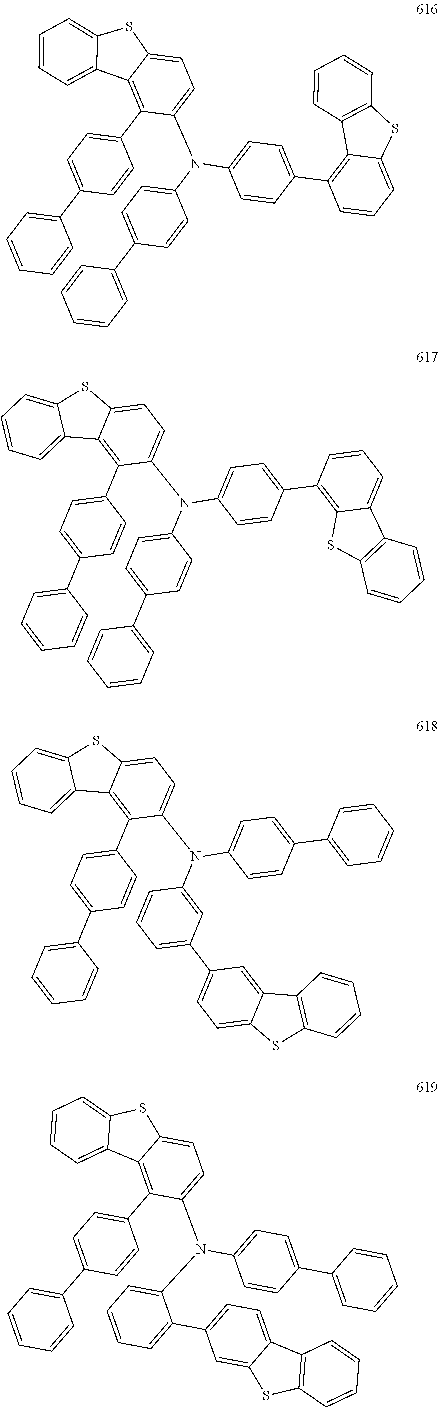 [SMILES: c1ccc(-c2ccc(-c3c(N(c4ccc(-c5ccccc5)cc4)c4ccc(-c5cccc6sc7ccccc7c56)cc4)ccc4sc5ccccc5c34)cc2)cc1 ; c1ccc(-c2ccc(-c3c(N(c4ccc(-c5ccccc5)cc4)c4ccc(-c5cccc6c5sc5ccccc56)cc4)ccc4sc5ccccc5c34)cc2)cc1 ; c1ccc(-c2ccc(-c3c(N(c4ccc(-c5ccccc5)cc4)c4cccc(-c5ccc6sc7ccccc7c6c5)c4)ccc4sc5ccccc5c34)cc2)cc1 ; c1ccc(-c2ccc(-c3c(N(c4ccc(-c5ccccc5)cc4)c4ccccc4-c4ccc5c(c4)sc4ccccc45)ccc4sc5ccccc5c34)cc2)cc1]

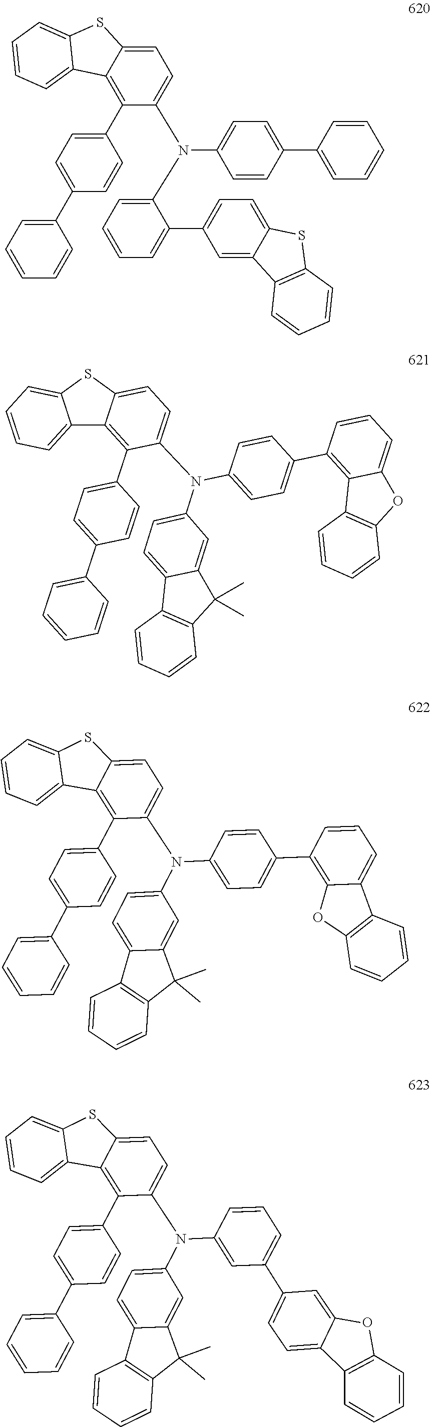 [SMILES: c1ccc(-c2ccc(-c3c(N(c4ccc(-c5ccccc5)cc4)c4ccccc4-c4ccc5sc6ccccc6c5c4)ccc4sc5ccccc5c34)cc2)cc1 ; CC1(C)c2ccccc2-c2ccc(N(c3ccc(-c4cccc5oc6ccccc6c45)cc3)c3ccc4sc5ccccc5c4c3-c3ccc(-c4ccccc4)cc3)cc21 ; CC1(C)c2ccccc2-c2ccc(N(c3ccc(-c4cccc5c4oc4ccccc45)cc3)c3ccc4sc5ccccc5c4c3-c3ccc(-c4ccccc4)cc3)cc21 ; CC1(C)c2ccccc2-c2ccc(N(c3cccc(-c4ccc5c(c4)oc4ccccc45)c3)c3ccc4sc5ccccc5c4c3-c3ccc(-c4ccccc4)cc3)cc21]

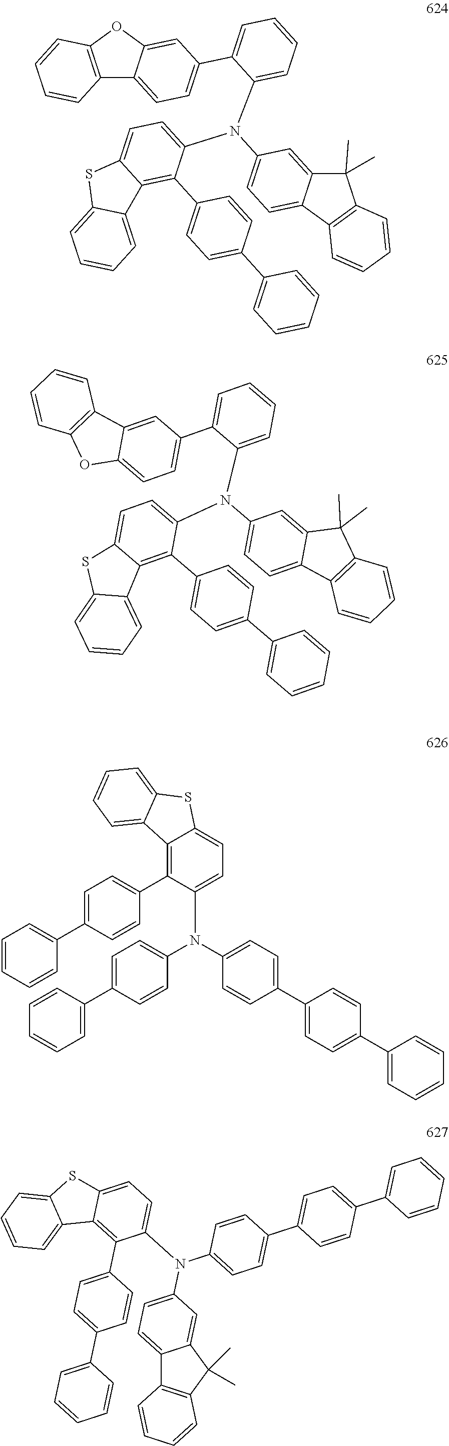 [SMILES: CC1(C)c2ccccc2-c2ccc(N(c3ccccc3-c3ccc4c(c3)oc3ccccc34)c3ccc4sc5ccccc5c4c3-c3ccc(-c4ccccc4)cc3)cc21 ; CC1(C)c2ccccc2-c2ccc(N(c3ccccc3-c3ccc4oc5ccccc5c4c3)c3ccc4sc5ccccc5c4c3-c3ccc(-c4ccccc4)cc3)cc21 ; c1ccc(-c2ccc(-c3ccc(N(c4ccc(-c5ccccc5)cc4)c4ccc5sc6ccccc6c5c4-c4ccc(-c5ccccc5)cc4)cc3)cc2)cc1 ; CC1(C)c2ccccc2-c2ccc(N(c3ccc(-c4ccc(-c5ccccc5)cc4)cc3)c3ccc4sc5ccccc5c4c3-c3ccc(-c4ccccc4)cc3)cc21]

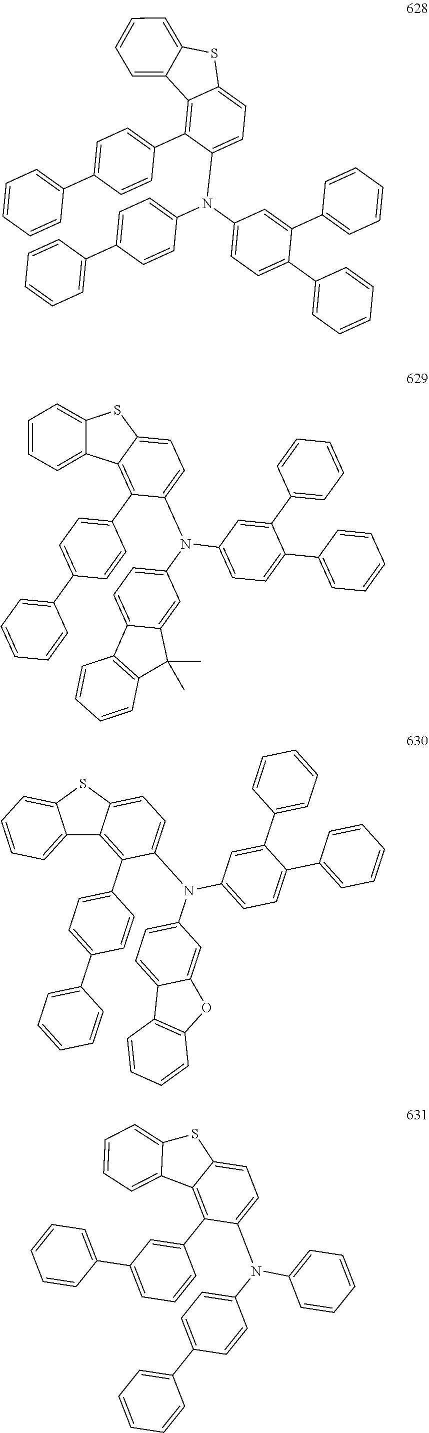 [SMILES: c1ccc(-c2ccc(-c3c(N(c4ccc(-c5ccccc5)cc4)c4ccc(-c5ccccc5)c(-c5ccccc5)c4)ccc4sc5ccccc5c34)cc2)cc1 ; CC1(C)c2ccccc2-c2ccc(N(c3ccc(-c4ccccc4)c(-c4ccccc4)c3)c3ccc4sc5ccccc5c4c3-c3ccc(-c4ccccc4)cc3)cc21 ; c1ccc(-c2ccc(-c3c(N(c4ccc(-c5ccccc5)c(-c5ccccc5)c4)c4ccc5c(c4)oc4ccccc45)ccc4sc5ccccc5c34)cc2)cc1 ; c1ccc(-c2ccc(N(c3ccccc3)c3ccc4sc5ccccc5c4c3-c3cccc(-c4ccccc4)c3)cc2)cc1]

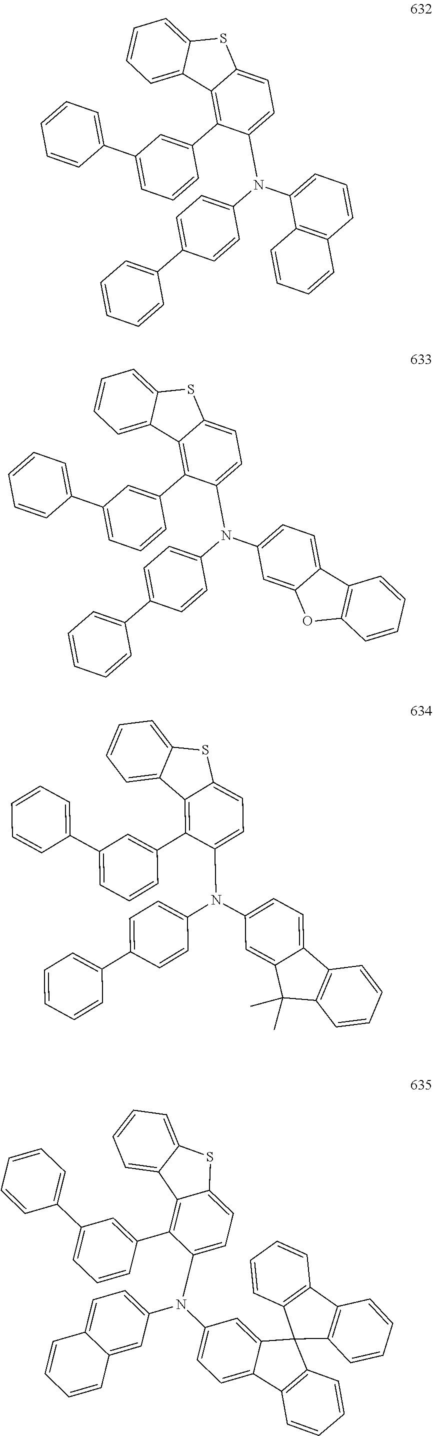

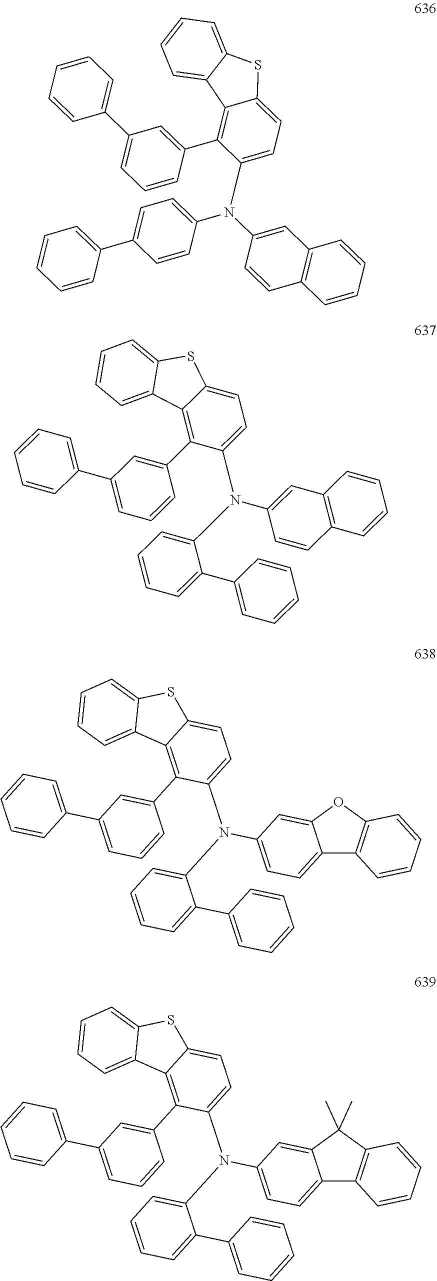 [SMILES: c1ccc(-c2ccc(N(c3ccc4ccccc4c3)c3ccc4sc5ccccc5c4c3-c3cccc(-c4ccccc4)c3)cc2)cc1 ; c1ccc(-c2cccc(-c3c(N(c4ccc5ccccc5c4)c4ccccc4-c4ccccc4)ccc4sc5ccccc5c34)c2)cc1 ; c1ccc(-c2cccc(-c3c(N(c4ccc5c(c4)oc4ccccc45)c4ccccc4-c4ccccc4)ccc4sc5ccccc5c34)c2)cc1 ; CC1(C)c2ccccc2-c2ccc(N(c3ccccc3-c3ccccc3)c3ccc4sc5ccccc5c4c3-c3cccc(-c4ccccc4)c3)cc21]

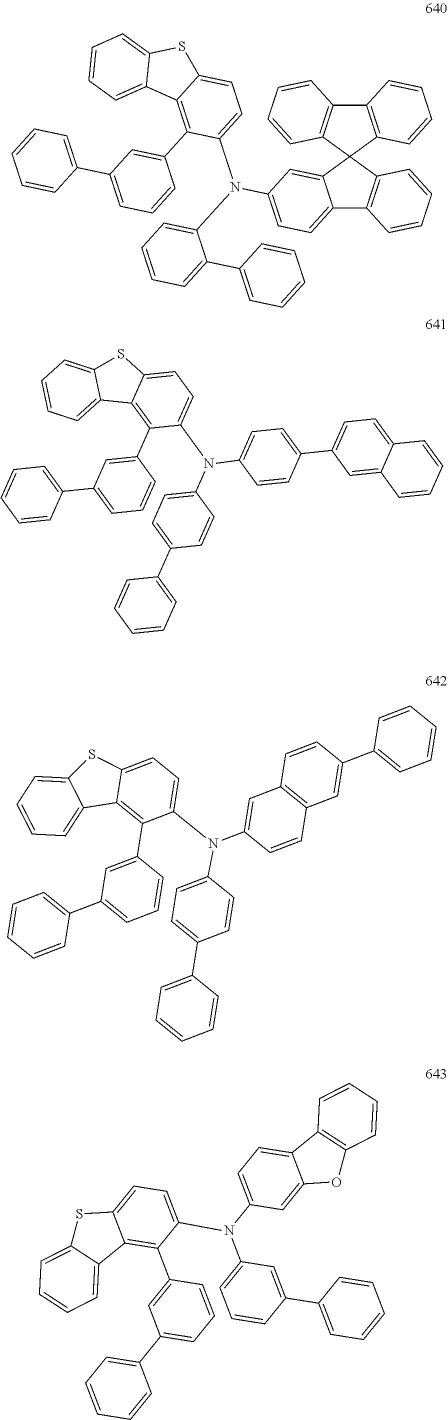 [SMILES: c1ccc(-c2cccc(-c3c(N(c4ccc5c(c4)C4(c6ccccc6-c6ccccc64)c4ccccc4-5)c4ccccc4-c4ccccc4)ccc4sc5ccccc5c34)c2)cc1 ; c1ccc(-c2ccc(N(c3ccc(-c4ccc5ccccc5c4)cc3)c3ccc4sc5ccccc5c4c3-c3cccc(-c4ccccc4)c3)cc2)cc1 ; c1ccc(-c2ccc(N(c3ccc4cc(-c5ccccc5)ccc4c3)c3ccc4sc5ccccc5c4c3-c3cccc(-c4ccccc4)c3)cc2)cc1 ; c1ccc(-c2cccc(-c3c(N(c4cccc(-c5ccccc5)c4)c4ccc5c(c4)oc4ccccc45)ccc4sc5ccccc5c34)c2)cc1]

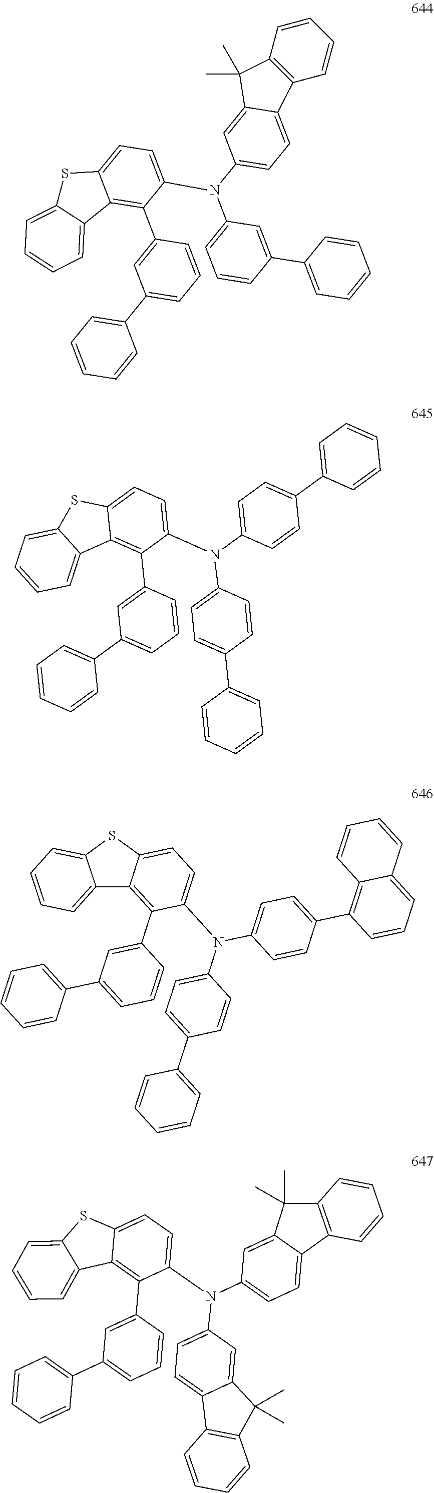 [SMILES: CC1(C)c2ccccc2-c2ccc(N(c3cccc(-c4ccccc4)c3)c3ccc4sc5ccccc5c4c3-c3cccc(-c4ccccc4)c3)cc21 ; c1ccc(-c2ccc(N(c3ccc(-c4ccccc4)cc3)c3ccc4sc5ccccc5c4c3-c3cccc(-c4ccccc4)c3)cc2)cc1 ; c1ccc(-c2ccc(N(c3ccc(-c4cccc5ccccc45)cc3)c3ccc4sc5ccccc5c4c3-c3cccc(-c4ccccc4)c3)cc2)cc1 ; CC1(C)c2ccccc2-c2ccc(N(c3ccc4c(c3)C(C)(C)c3ccccc3-4)c3ccc4sc5ccccc5c4c3-c3cccc(-c4ccccc4)c3)cc21]

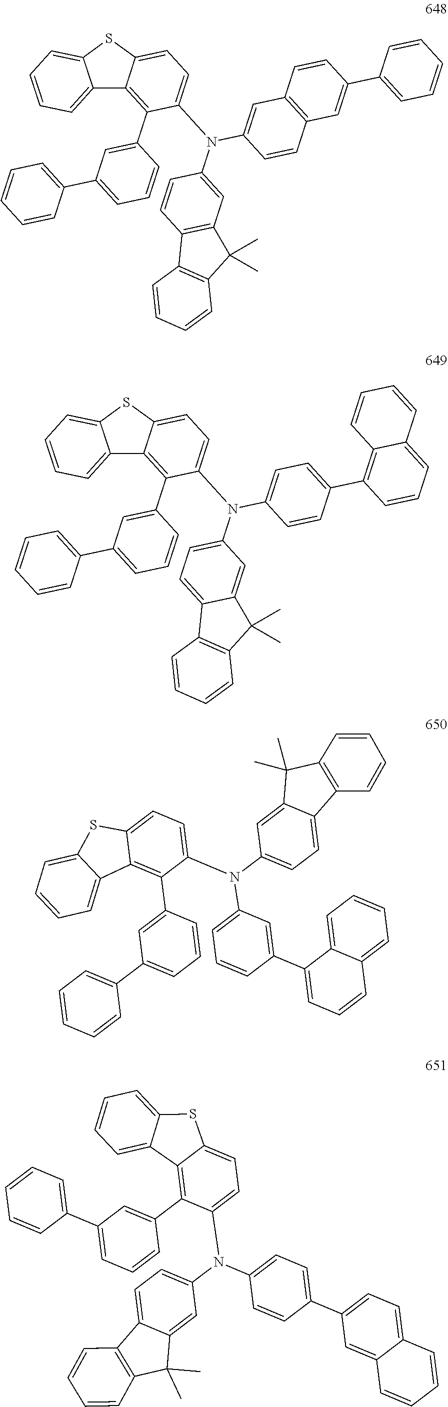 [SMILES: CC1(C)c2ccccc2-c2ccc(N(c3ccc4cc(-c5ccccc5)ccc4c3)c3ccc4sc5ccccc5c4c3-c3cccc(-c4ccccc4)c3)cc21 ; CC1(C)c2ccccc2-c2ccc(N(c3ccc(-c4cccc5ccccc45)cc3)c3ccc4sc5ccccc5c4c3-c3cccc(-c4ccccc4)c3)cc21 ; CC1(C)c2ccccc2-c2ccc(N(c3cccc(-c4cccc5ccccc45)c3)c3ccc4sc5ccccc5c4c3-c3cccc(-c4ccccc4)c3)cc21 ; CC1(C)c2ccccc2-c2ccc(N(c3ccc(-c4ccc5ccccc5c4)cc3)c3ccc4sc5ccccc5c4c3-c3cccc(-c4ccccc4)c3)cc21]

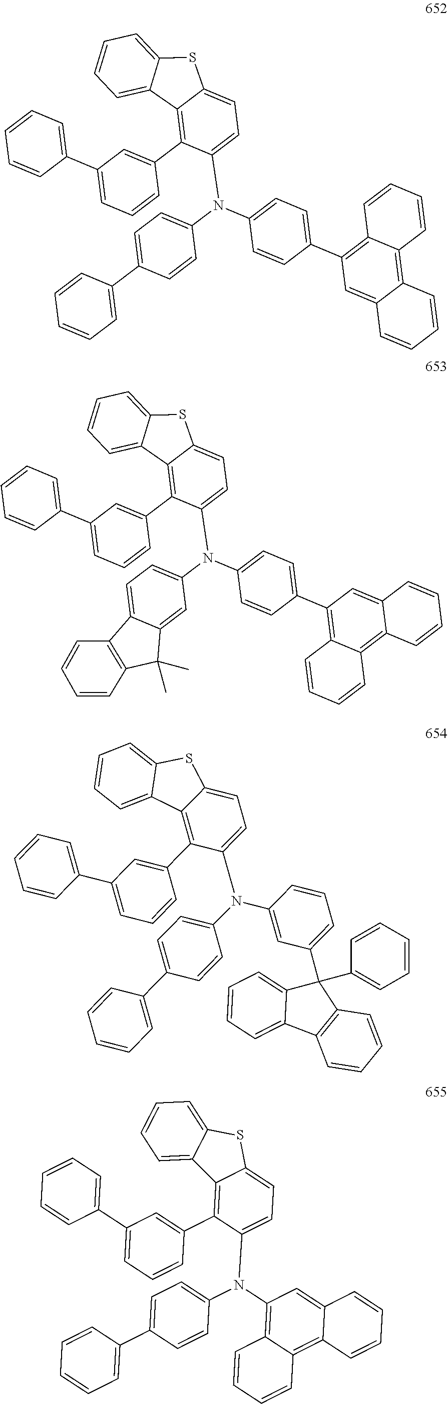 [SMILES: c1ccc(-c2ccc(N(c3ccc(-c4cc5ccccc5c5ccccc45)cc3)c3ccc4sc5ccccc5c4c3-c3cccc(-c4ccccc4)c3)cc2)cc1 ; CC1(C)c2ccccc2-c2ccc(N(c3ccc(-c4cc5ccccc5c5ccccc45)cc3)c3ccc4sc5ccccc5c4c3-c3cccc(-c4ccccc4)c3)cc21 ; c1ccc(-c2ccc(N(c3cccc(C4(c5ccccc5)c5ccccc5-c5ccccc54)c3)c3ccc4sc5ccccc5c4c3-c3cccc(-c4ccccc4)c3)cc2)cc1 ; c1ccc(-c2ccc(N(c3ccc4sc5ccccc5c4c3-c3cccc(-c4ccccc4)c3)c3cc4ccccc4c4ccccc34)cc2)cc1]

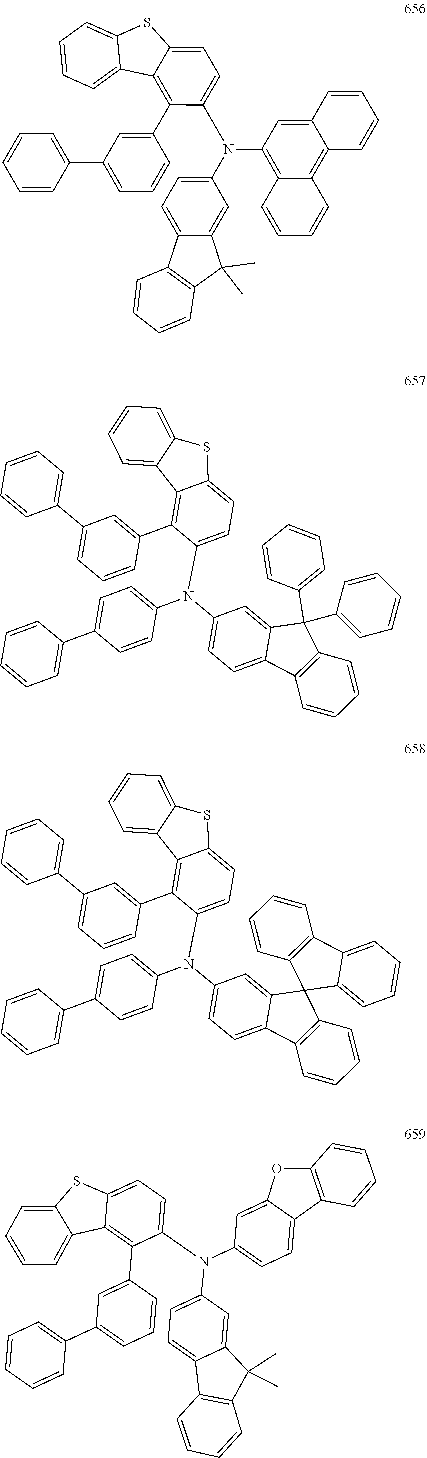 [SMILES: CC1(C)c2ccccc2-c2ccc(N(c3ccc4sc5ccccc5c4c3-c3cccc(-c4ccccc4)c3)c3cc4ccccc4c4ccccc34)cc21 ; c1ccc(-c2ccc(N(c3ccc4c(c3)C(c3ccccc3)(c3ccccc3)c3ccccc3-4)c3ccc4sc5ccccc5c4c3-c3cccc(-c4ccccc4)c3)cc2)cc1 ; c1ccc(-c2ccc(N(c3ccc4c(c3)C3(c5ccccc5-c5ccccc53)c3ccccc3-4)c3ccc4sc5ccccc5c4c3-c3cccc(-c4ccccc4)c3)cc2)cc1 ; CC1(C)c2ccccc2-c2ccc(N(c3ccc4c(c3)oc3ccccc34)c3ccc4sc5ccccc5c4c3-c3cccc(-c4ccccc4)c3)cc21]

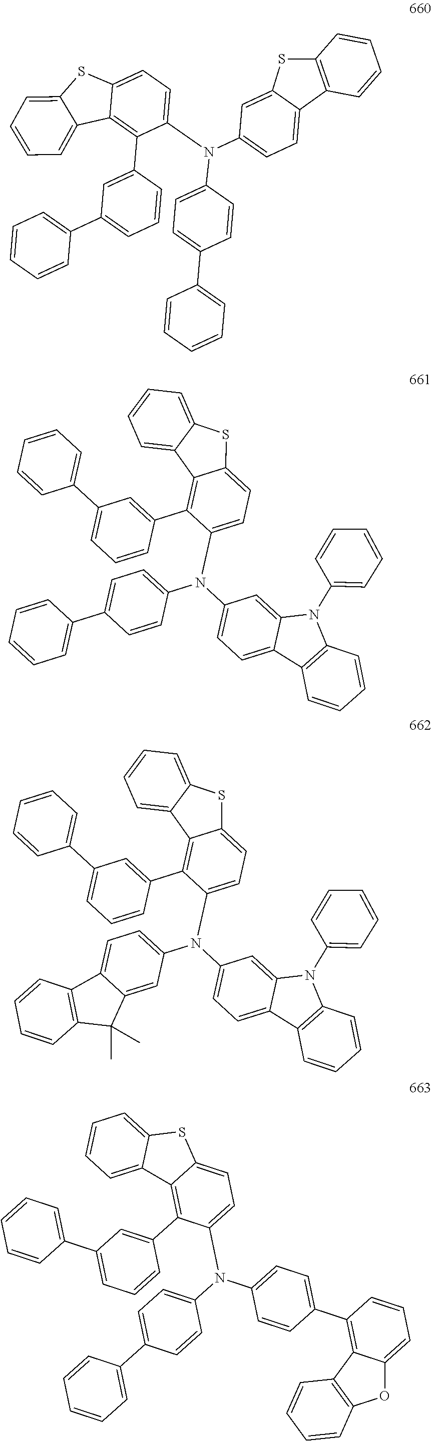

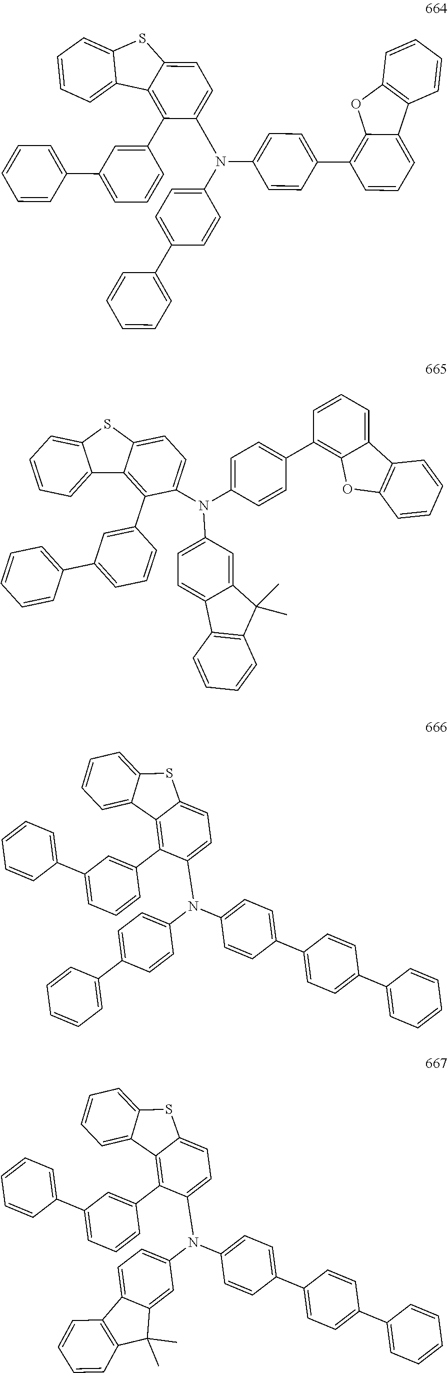 [SMILES: c1ccc(-c2ccc(N(c3ccc(-c4cccc5c4oc4ccccc45)cc3)c3ccc4sc5ccccc5c4c3-c3cccc(-c4ccccc4)c3)cc2)cc1 ; CC1(C)c2ccccc2-c2ccc(N(c3ccc(-c4cccc5c4oc4ccccc45)cc3)c3ccc4sc5ccccc5c4c3-c3cccc(-c4ccccc4)c3)cc21 ; c1ccc(-c2ccc(-c3ccc(N(c4ccc(-c5ccccc5)cc4)c4ccc5sc6ccccc6c5c4-c4cccc(-c5ccccc5)c4)cc3)cc2)cc1 ; CC1(C)c2ccccc2-c2ccc(N(c3ccc(-c4ccc(-c5ccccc5)cc4)cc3)c3ccc4sc5ccccc5c4c3-c3cccc(-c4ccccc4)c3)cc21]

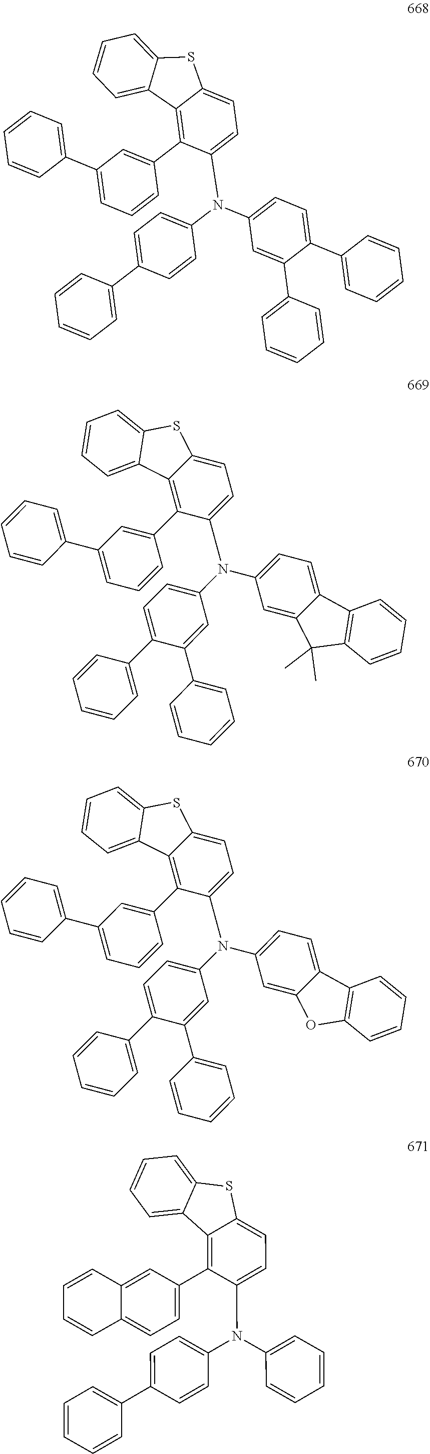

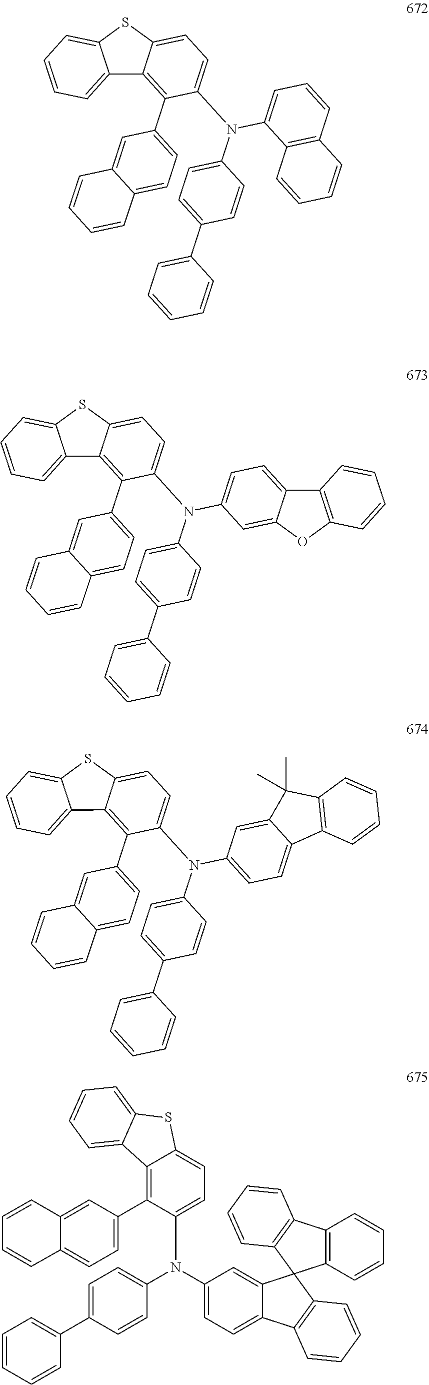

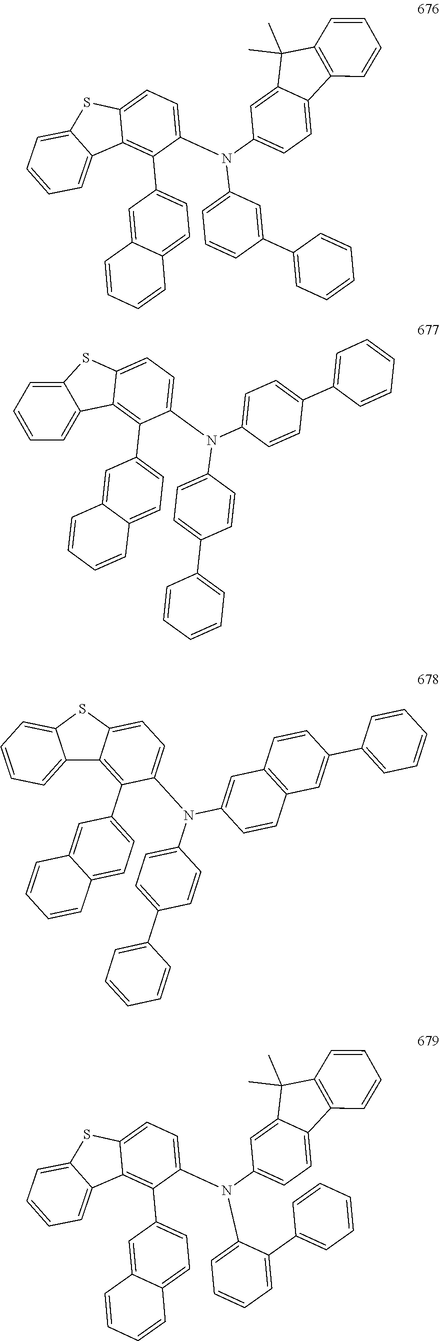 [SMILES: CC1(C)c2ccccc2-c2ccc(N(c3cccc(-c4ccccc4)c3)c3ccc4sc5ccccc5c4c3-c3ccc4ccccc4c3)cc21 ; c1ccc(-c2ccc(N(c3ccc(-c4ccccc4)cc3)c3ccc4sc5ccccc5c4c3-c3ccc4ccccc4c3)cc2)cc1 ; c1ccc(-c2ccc(N(c3ccc4cc(-c5ccccc5)ccc4c3)c3ccc4sc5ccccc5c4c3-c3ccc4ccccc4c3)cc2)cc1 ; CC1(C)c2ccccc2-c2ccc(N(c3ccccc3-c3ccccc3)c3ccc4sc5ccccc5c4c3-c3ccc4ccccc4c3)cc21]

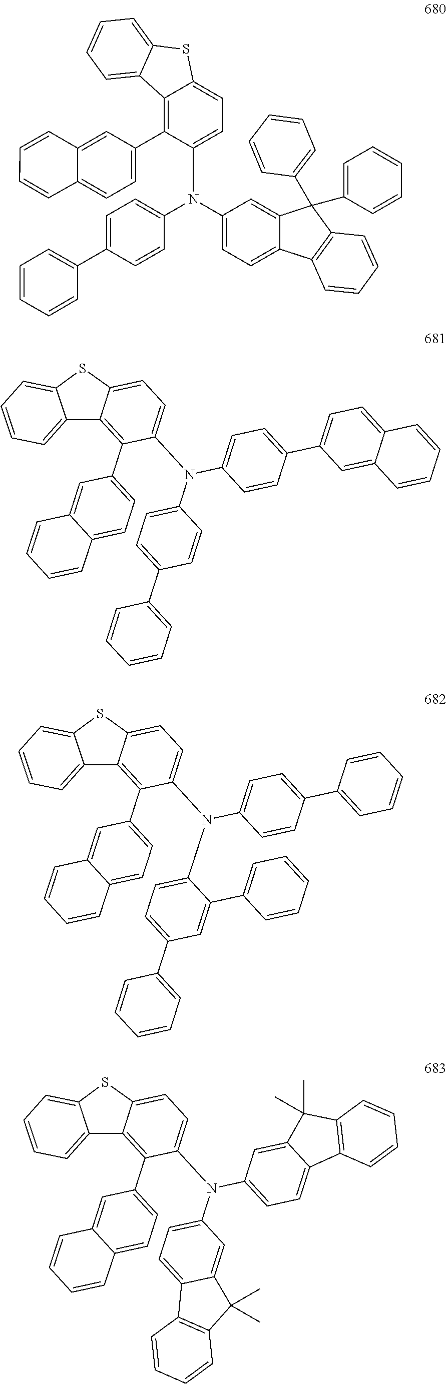 [SMILES: c1ccc(-c2ccc(N(c3ccc4c(c3)C(c3ccccc3)(c3ccccc3)c3ccccc3-4)c3ccc4sc5ccccc5c4c3-c3ccc4ccccc4c3)cc2)cc1 ; c1ccc(-c2ccc(N(c3ccc(-c4ccc5ccccc5c4)cc3)c3ccc4sc5ccccc5c4c3-c3ccc4ccccc4c3)cc2)cc1 ; c1ccc(-c2ccc(N(c3ccc(-c4ccccc4)cc3-c3ccccc3)c3ccc4sc5ccccc5c4c3-c3ccc4ccccc4c3)cc2)cc1 ; CC1(C)c2ccccc2-c2ccc(N(c3ccc4c(c3)C(C)(C)c3ccccc3-4)c3ccc4sc5ccccc5c4c3-c3ccc4ccccc4c3)cc21]

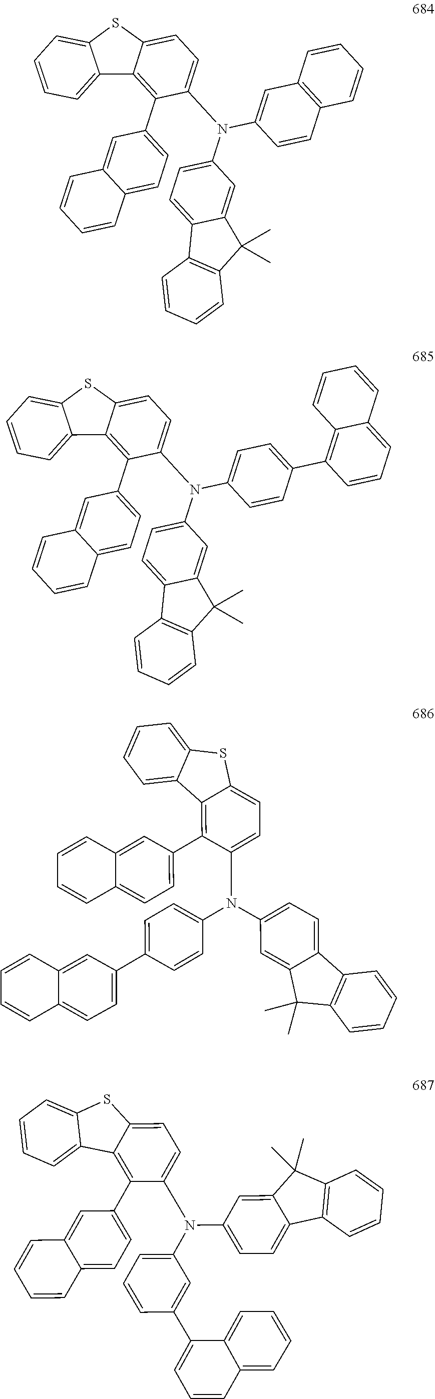

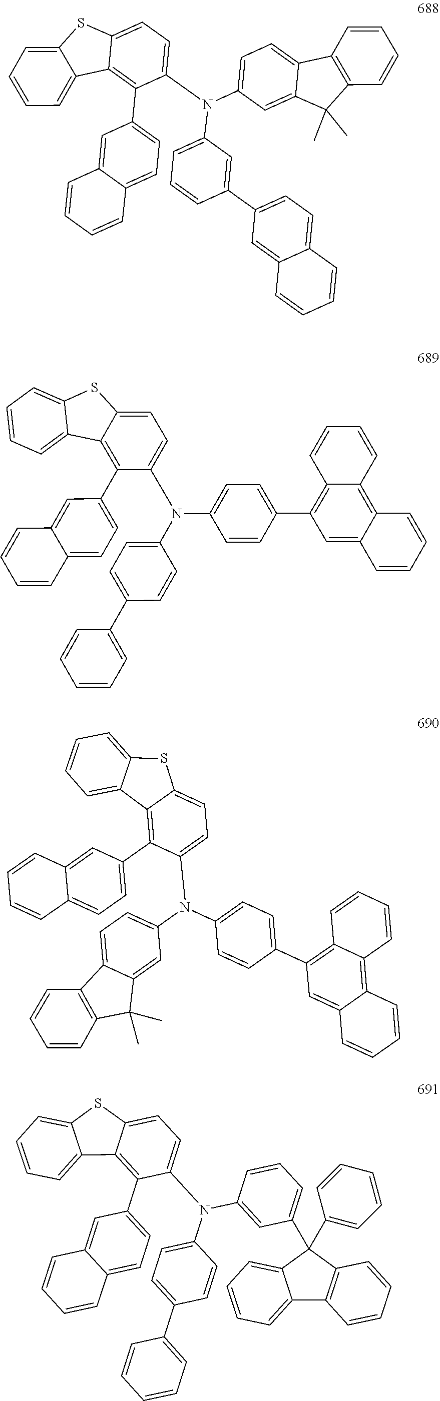

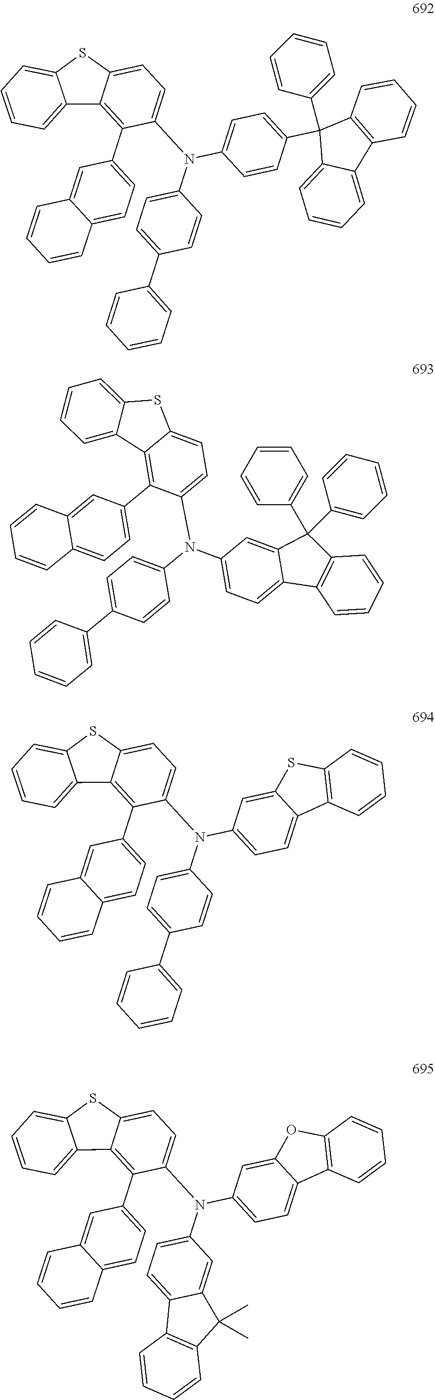

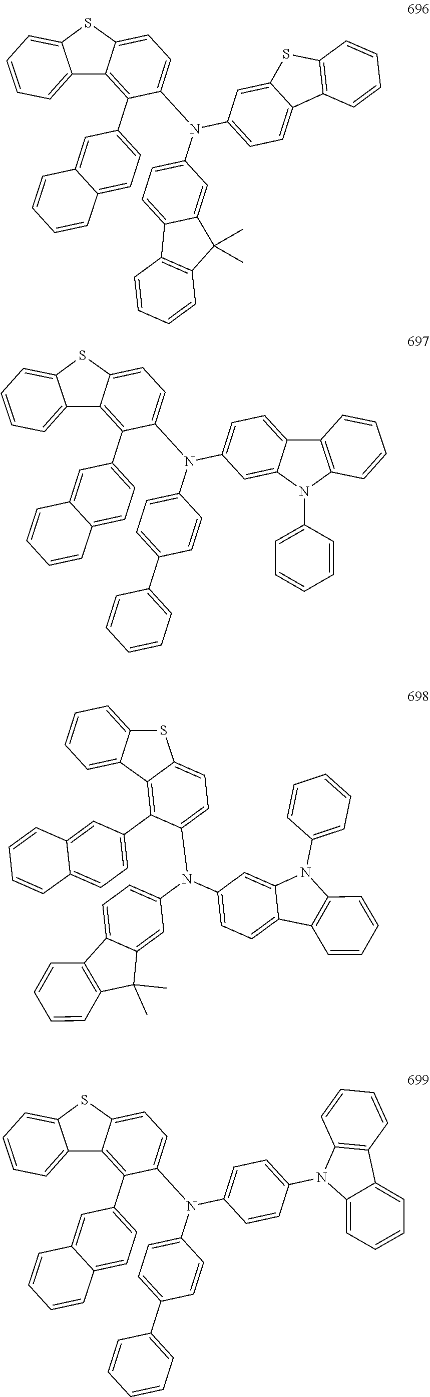 [SMILES: CC1(C)c2ccccc2-c2ccc(N(c3ccc4c(c3)sc3ccccc34)c3ccc4sc5ccccc5c4c3-c3ccc4ccccc4c3)cc21 ; c1ccc(-c2ccc(N(c3ccc4c5ccccc5n(-c5ccccc5)c4c3)c3ccc4sc5ccccc5c4c3-c3ccc4ccccc4c3)cc2)cc1 ; CC1(C)c2ccccc2-c2ccc(N(c3ccc4c5ccccc5n(-c5ccccc5)c4c3)c3ccc4sc5ccccc5c4c3-c3ccc4ccccc4c3)cc21 ; c1ccc(-c2ccc(N(c3ccc(-n4c5ccccc5c5ccccc54)cc3)c3ccc4sc5ccccc5c4c3-c3ccc4ccccc4c3)cc2)cc1]

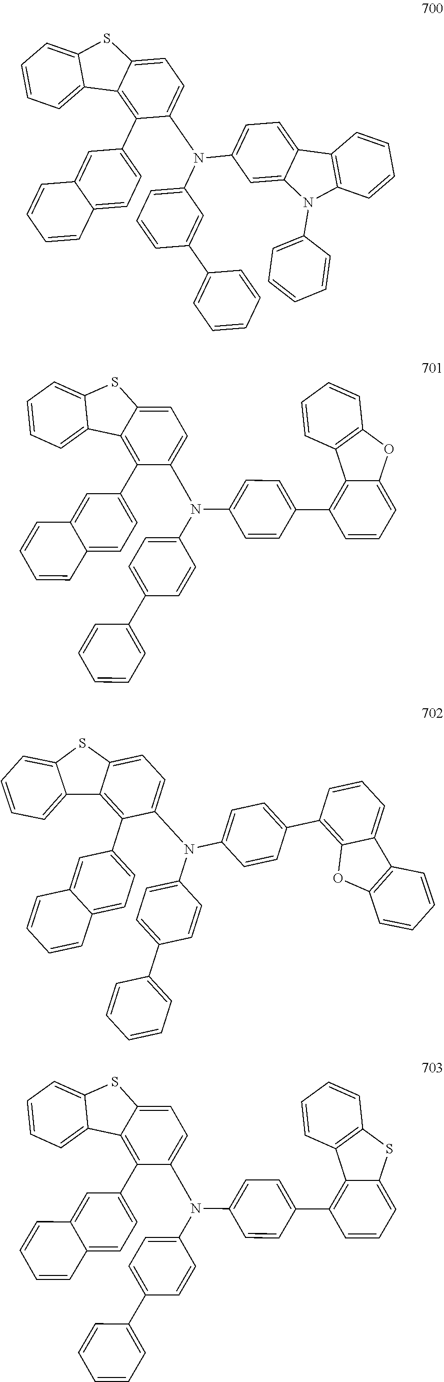

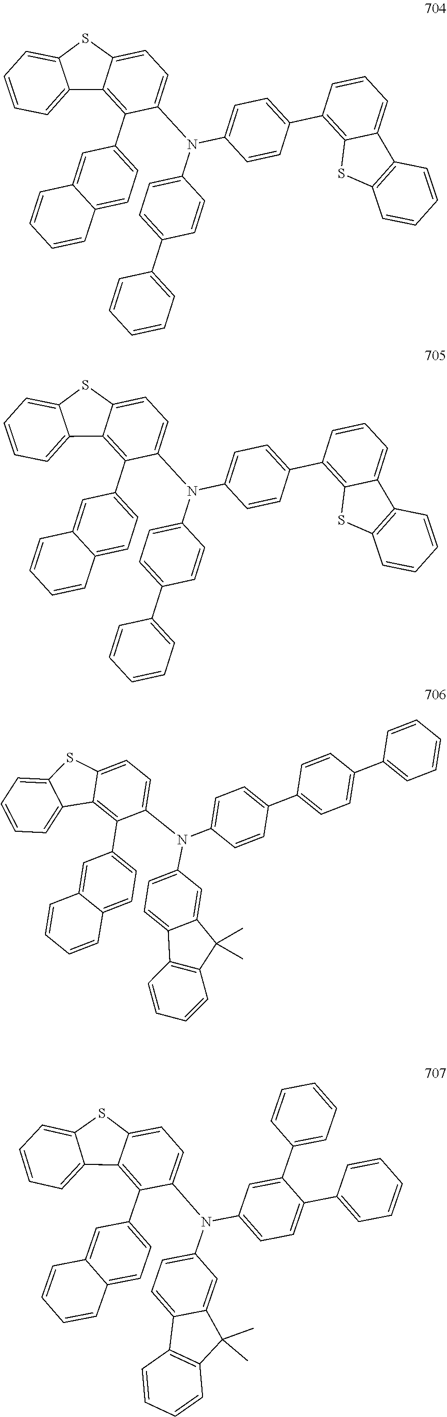 [SMILES: c1ccc(-c2ccc(N(c3ccc(-c4cccc5c4sc4ccccc45)cc3)c3ccc4sc5ccccc5c4c3-c3ccc4ccccc4c3)cc2)cc1 ; c1ccc(-c2ccc(N(c3ccc(-c4cccc5c4sc4ccccc45)cc3)c3ccc4sc5ccccc5c4c3-c3ccc4ccccc4c3)cc2)cc1 ; CC1(C)c2ccccc2-c2ccc(N(c3ccc(-c4ccc(-c5ccccc5)cc4)cc3)c3ccc4sc5ccccc5c4c3-c3ccc4ccccc4c3)cc21 ; CC1(C)c2ccccc2-c2ccc(N(c3ccc(-c4ccccc4)c(-c4ccccc4)c3)c3ccc4sc5ccccc5c4c3-c3ccc4ccccc4c3)cc21]

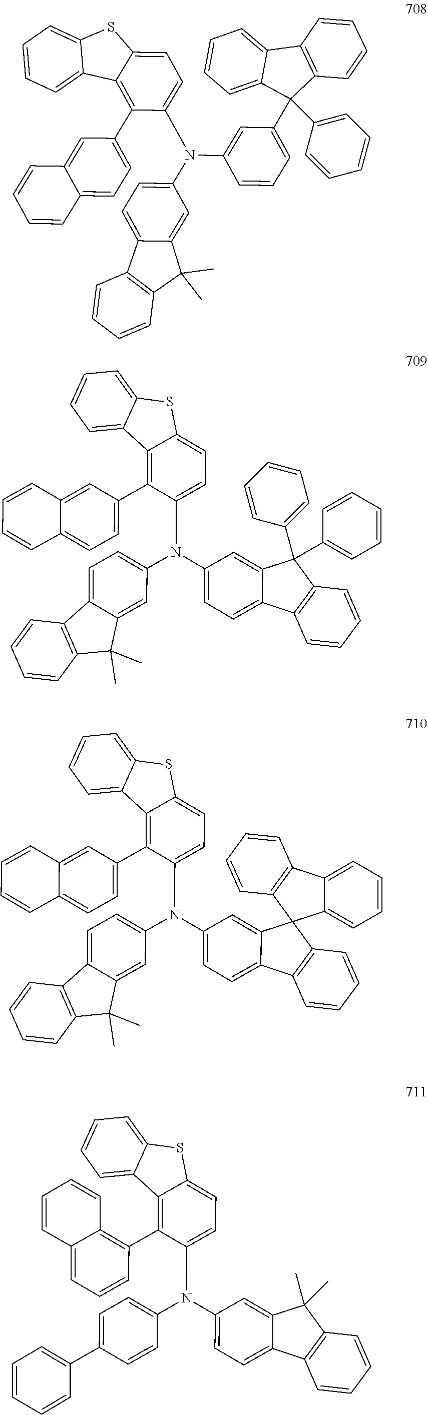

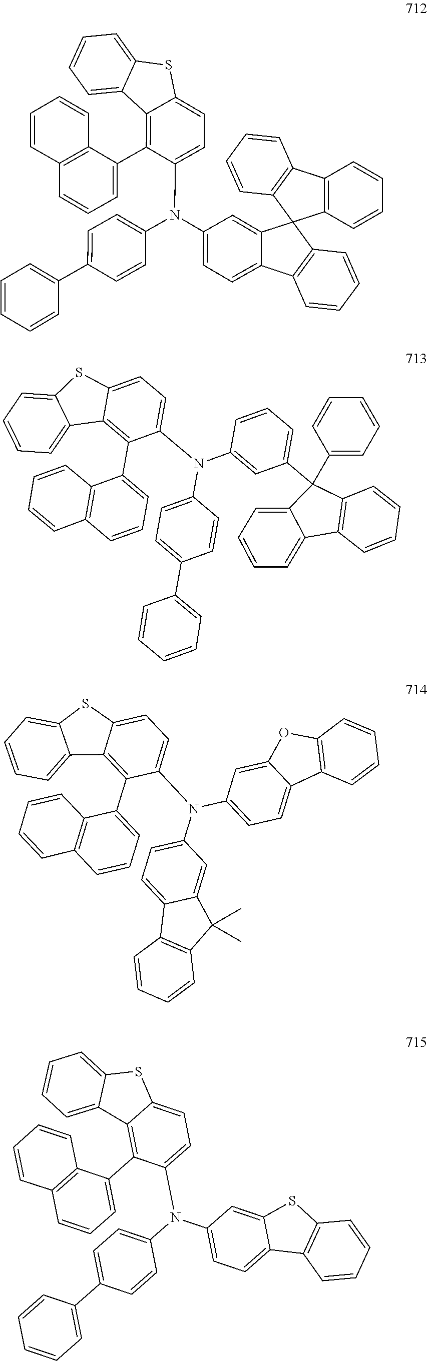 [SMILES: c1ccc(-c2ccc(N(c3ccc4c(c3)C3(c5ccccc5-c5ccccc53)c3ccccc3-4)c3ccc4sc5ccccc5c4c3-c3cccc4ccccc34)cc2)cc1 ; c1ccc(-c2ccc(N(c3cccc(C4(c5ccccc5)c5ccccc5-c5ccccc54)c3)c3ccc4sc5ccccc5c4c3-c3cccc4ccccc34)cc2)cc1 ; CC1(C)c2ccccc2-c2ccc(N(c3ccc4c(c3)oc3ccccc34)c3ccc4sc5ccccc5c4c3-c3cccc4ccccc34)cc21 ; c1ccc(-c2ccc(N(c3ccc4c(c3)sc3ccccc34)c3ccc4sc5ccccc5c4c3-c3cccc4ccccc34)cc2)cc1]

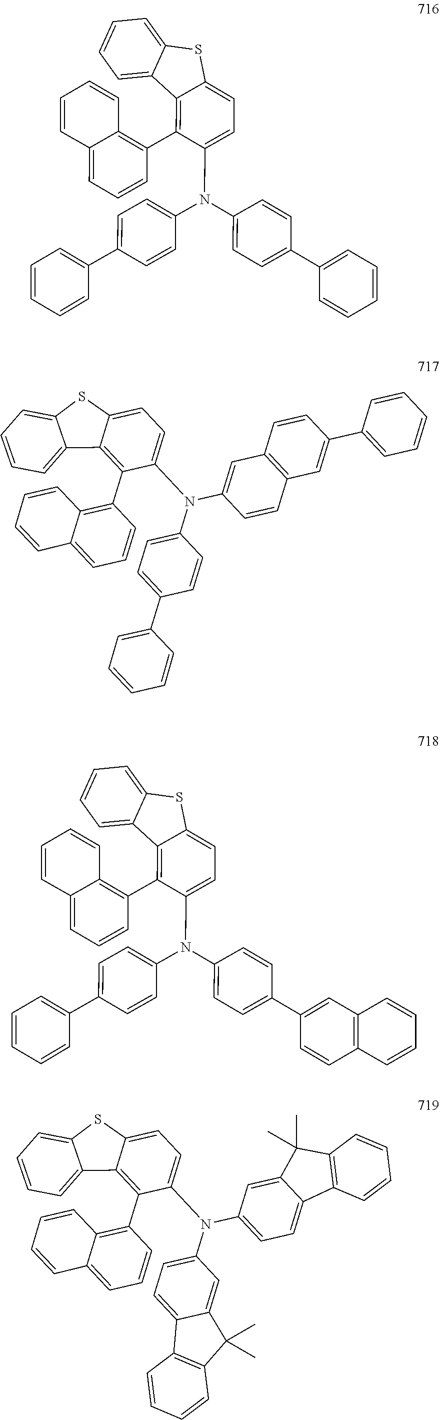 [SMILES: c1ccc(-c2ccc(N(c3ccc(-c4ccccc4)cc3)c3ccc4sc5ccccc5c4c3-c3cccc4ccccc34)cc2)cc1 ; c1ccc(-c2ccc(N(c3ccc4cc(-c5ccccc5)ccc4c3)c3ccc4sc5ccccc5c4c3-c3cccc4ccccc34)cc2)cc1 ; c1ccc(-c2ccc(N(c3ccc(-c4ccc5ccccc5c4)cc3)c3ccc4sc5ccccc5c4c3-c3cccc4ccccc34)cc2)cc1 ; CC1(C)c2ccccc2-c2ccc(N(c3ccc4c(c3)C(C)(C)c3ccccc3-4)c3ccc4sc5ccccc5c4c3-c3cccc4ccccc34)cc21]

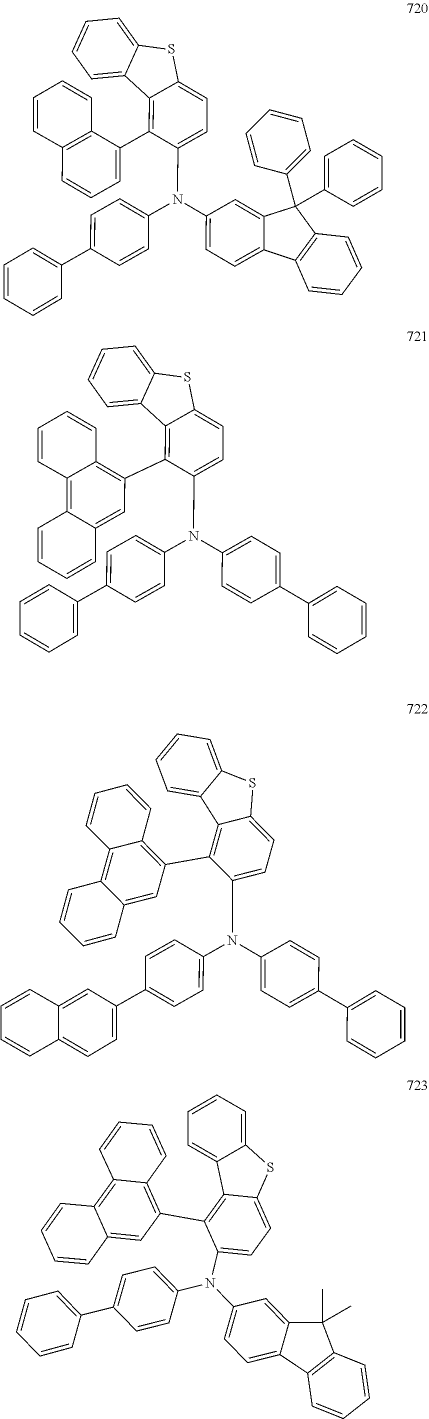 [SMILES: c1ccc(-c2ccc(N(c3ccc4c(c3)C(c3ccccc3)(c3ccccc3)c3ccccc3-4)c3ccc4sc5ccccc5c4c3-c3cccc4ccccc34)cc2)cc1 ; c1ccc(-c2ccc(N(c3ccc(-c4ccccc4)cc3)c3ccc4sc5ccccc5c4c3-c3cc4ccccc4c4ccccc34)cc2)cc1 ; c1ccc(-c2ccc(N(c3ccc(-c4ccc5ccccc5c4)cc3)c3ccc4sc5ccccc5c4c3-c3cc4ccccc4c4ccccc34)cc2)cc1 ; CC1(C)c2ccccc2-c2ccc(N(c3ccc(-c4ccccc4)cc3)c3ccc4sc5ccccc5c4c3-c3cc4ccccc4c4ccccc34)cc21]

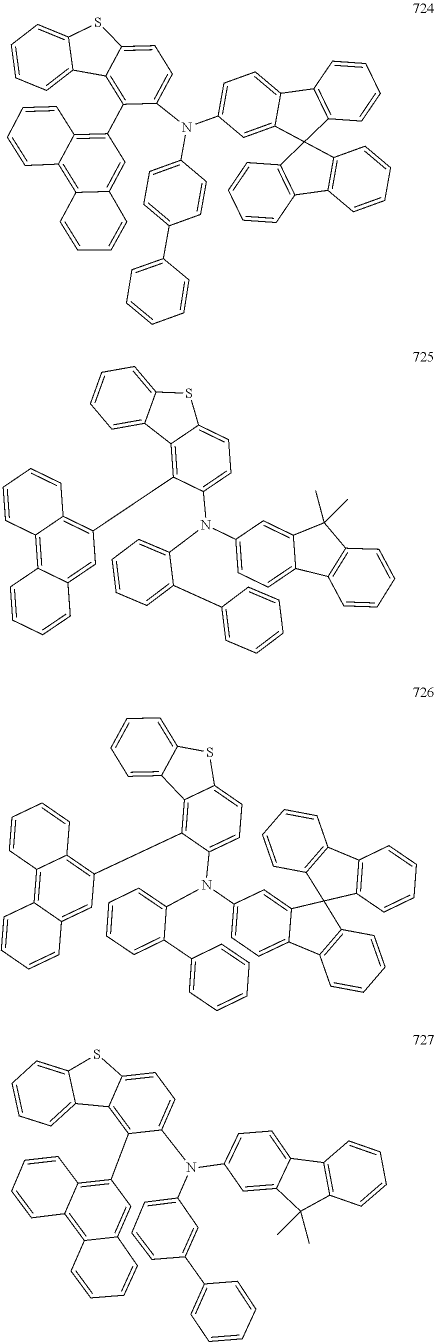

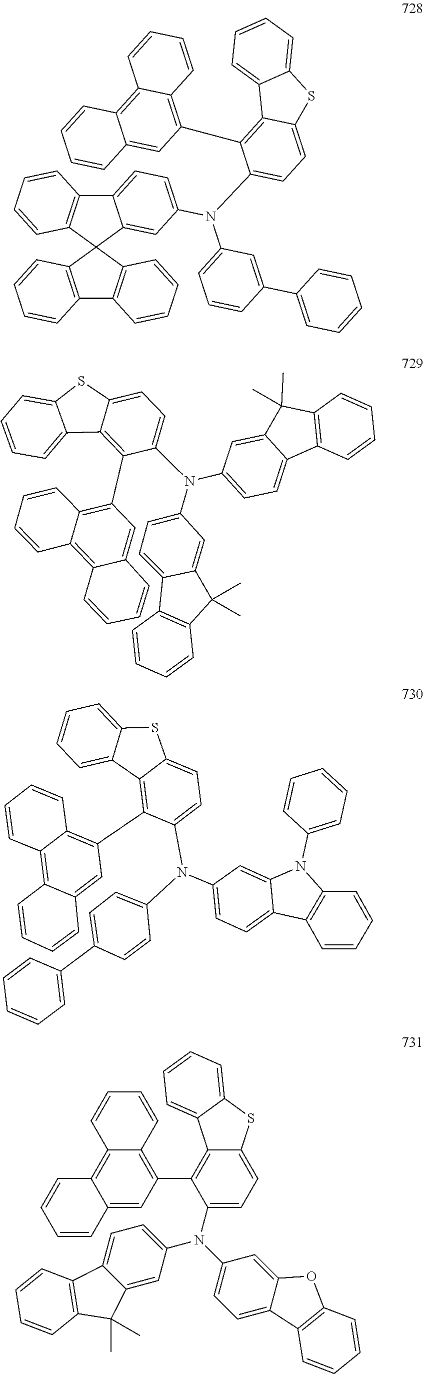 [SMILES: c1ccc(-c2cccc(N(c3ccc4c(c3)C3(c5ccccc5-c5ccccc53)c3ccccc3-4)c3ccc4sc5ccccc5c4c3-c3cc4ccccc4c4ccccc34)c2)cc1 ; CC1(C)c2ccccc2-c2ccc(N(c3ccc4c(c3)C(C)(C)c3ccccc3-4)c3ccc4sc5ccccc5c4c3-c3cc4ccccc4c4ccccc34)cc21 ; c1ccc(-c2ccc(N(c3ccc4c5ccccc5n(-c5ccccc5)c4c3)c3ccc4sc5ccccc5c4c3-c3cc4ccccc4c4ccccc34)cc2)cc1 ; CC1(C)c2ccccc2-c2ccc(N(c3ccc4c(c3)oc3ccccc34)c3ccc4sc5ccccc5c4c3-c3cc4ccccc4c4ccccc34)cc21]

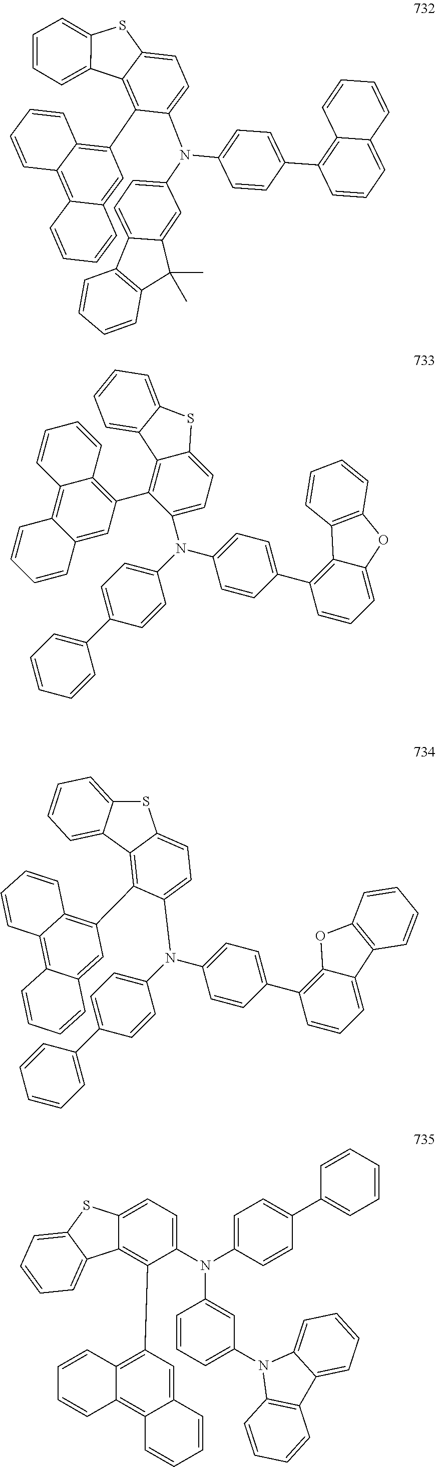

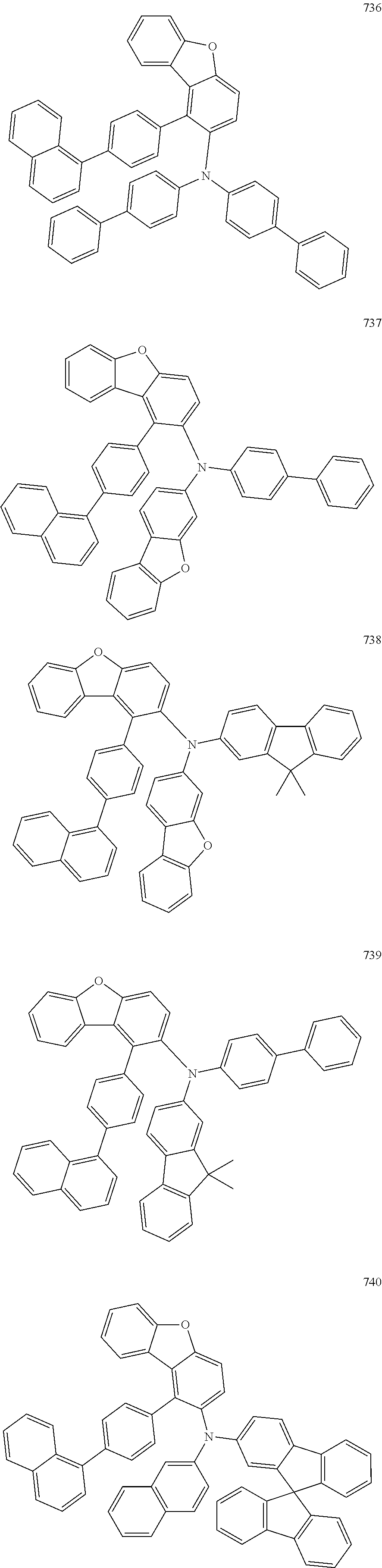 [SMILES: c1ccc(-c2ccc(N(c3ccc(-c4ccccc4)cc3)c3ccc4oc5ccccc5c4c3-c3ccc(-c4cccc5ccccc45)cc3)cc2)cc1 ; c1ccc(-c2ccc(N(c3ccc4c(c3)oc3ccccc34)c3ccc4oc5ccccc5c4c3-c3ccc(-c4cccc5ccccc45)cc3)cc2)cc1 ; CC1(C)c2ccccc2-c2ccc(N(c3ccc4c(c3)oc3ccccc34)c3ccc4oc5ccccc5c4c3-c3ccc(-c4cccc5ccccc45)cc3)cc21 ; CC1(C)c2ccccc2-c2ccc(N(c3ccc(-c4ccccc4)cc3)c3ccc4oc5ccccc5c4c3-c3ccc(-c4cccc5ccccc45)cc3)cc21 ; c1ccc2c(c1)-c1ccccc1C21c2ccccc2-c2ccc(N(c3ccc4ccccc4c3)c3ccc4oc5ccccc5c4c3-c3ccc(-c4cccc5ccccc45)cc3)cc21]

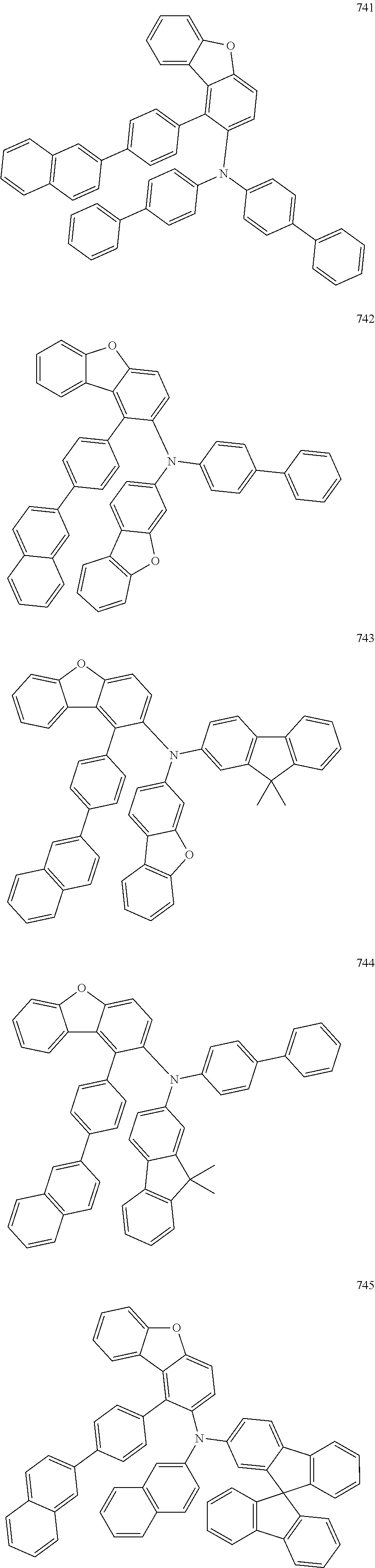 [SMILES: c1ccc(-c2ccc(N(c3ccc(-c4ccccc4)cc3)c3ccc4oc5ccccc5c4c3-c3ccc(-c4ccc5ccccc5c4)cc3)cc2)cc1 ; c1ccc(-c2ccc(N(c3ccc4c(c3)oc3ccccc34)c3ccc4oc5ccccc5c4c3-c3ccc(-c4ccc5ccccc5c4)cc3)cc2)cc1 ; CC1(C)c2ccccc2-c2ccc(N(c3ccc4c(c3)oc3ccccc34)c3ccc4oc5ccccc5c4c3-c3ccc(-c4ccc5ccccc5c4)cc3)cc21 ; CC1(C)c2ccccc2-c2ccc(N(c3ccc(-c4ccccc4)cc3)c3ccc4oc5ccccc5c4c3-c3ccc(-c4ccc5ccccc5c4)cc3)cc21 ; c1ccc2c(c1)-c1ccccc1C21c2ccccc2-c2ccc(N(c3ccc4ccccc4c3)c3ccc4oc5ccccc5c4c3-c3ccc(-c4ccc5ccccc5c4)cc3)cc21]

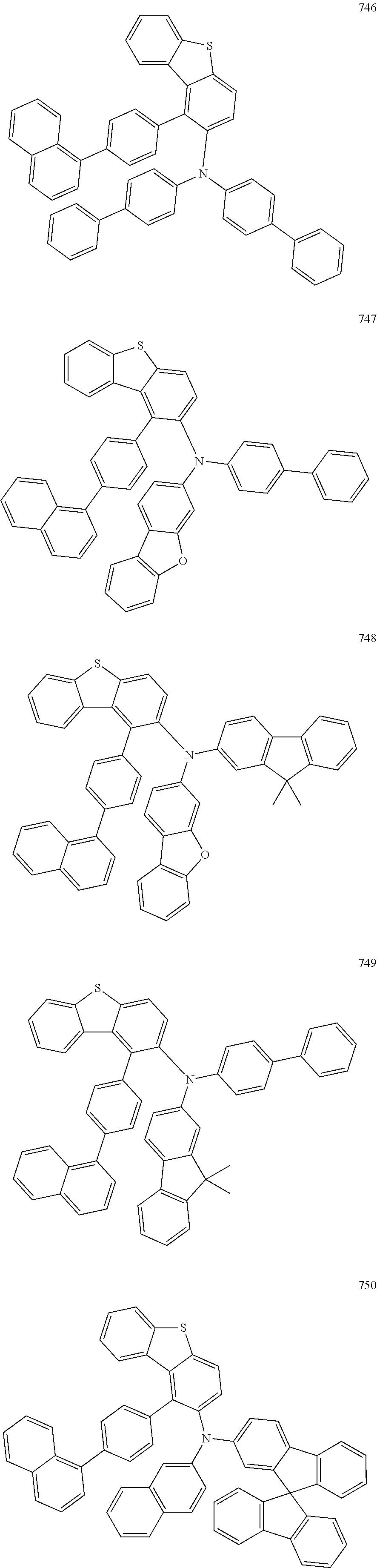 [SMILES: c1ccc(-c2ccc(N(c3ccc(-c4ccccc4)cc3)c3ccc4sc5ccccc5c4c3-c3ccc(-c4cccc5ccccc45)cc3)cc2)cc1 ; c1ccc(-c2ccc(N(c3ccc4c(c3)oc3ccccc34)c3ccc4sc5ccccc5c4c3-c3ccc(-c4cccc5ccccc45)cc3)cc2)cc1 ; CC1(C)c2ccccc2-c2ccc(N(c3ccc4c(c3)oc3ccccc34)c3ccc4sc5ccccc5c4c3-c3ccc(-c4cccc5ccccc45)cc3)cc21 ; CC1(C)c2ccccc2-c2ccc(N(c3ccc(-c4ccccc4)cc3)c3ccc4sc5ccccc5c4c3-c3ccc(-c4cccc5ccccc45)cc3)cc21 ; c1ccc2c(c1)-c1ccccc1C21c2ccccc2-c2ccc(N(c3ccc4ccccc4c3)c3ccc4sc5ccccc5c4c3-c3ccc(-c4cccc5ccccc45)cc3)cc21]

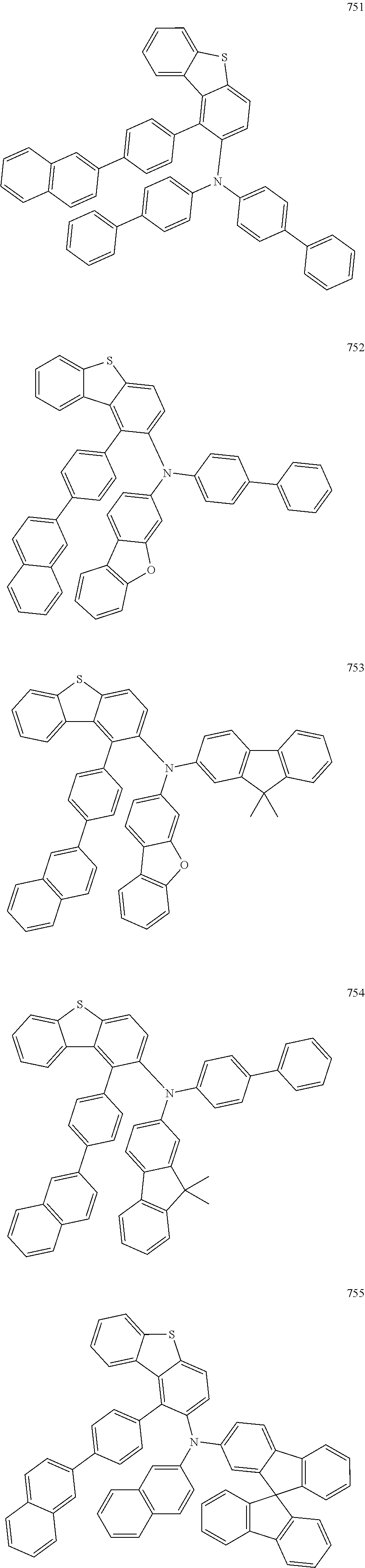 [SMILES: c1ccc(-c2ccc(N(c3ccc(-c4ccccc4)cc3)c3ccc4sc5ccccc5c4c3-c3ccc(-c4ccc5ccccc5c4)cc3)cc2)cc1 ; c1ccc(-c2ccc(N(c3ccc4c(c3)oc3ccccc34)c3ccc4sc5ccccc5c4c3-c3ccc(-c4ccc5ccccc5c4)cc3)cc2)cc1 ; CC1(C)c2ccccc2-c2ccc(N(c3ccc4c(c3)oc3ccccc34)c3ccc4sc5ccccc5c4c3-c3ccc(-c4ccc5ccccc5c4)cc3)cc21 ; CC1(C)c2ccccc2-c2ccc(N(c3ccc(-c4ccccc4)cc3)c3ccc4sc5ccccc5c4c3-c3ccc(-c4ccc5ccccc5c4)cc3)cc21 ; c1ccc2c(c1)-c1ccccc1C21c2ccccc2-c2ccc(N(c3ccc4ccccc4c3)c3ccc4sc5ccccc5c4c3-c3ccc(-c4ccc5ccccc5c4)cc3)cc21]

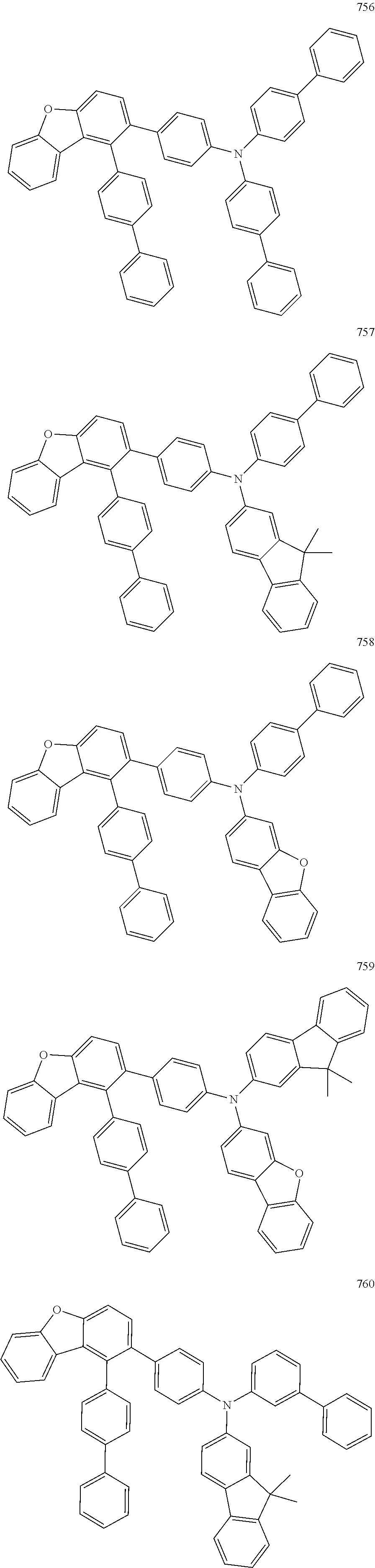 [SMILES: c1ccc(-c2ccc(-c3c(-c4ccc(N(c5ccc(-c6ccccc6)cc5)c5ccc(-c6ccccc6)cc5)cc4)ccc4oc5ccccc5c34)cc2)cc1 ; CC1(C)c2ccccc2-c2ccc(N(c3ccc(-c4ccccc4)cc3)c3ccc(-c4ccc5oc6ccccc6c5c4-c4ccc(-c5ccccc5)cc4)cc3)cc21 ; c1ccc(-c2ccc(-c3c(-c4ccc(N(c5ccc(-c6ccccc6)cc5)c5ccc6c(c5)oc5ccccc56)cc4)ccc4oc5ccccc5c34)cc2)cc1 ; CC1(C)c2ccccc2-c2ccc(N(c3ccc(-c4ccc5oc6ccccc6c5c4-c4ccc(-c5ccccc5)cc4)cc3)c3ccc4c(c3)oc3ccccc34)cc21 ; CC1(C)c2ccccc2-c2ccc(N(c3ccc(-c4ccc5oc6ccccc6c5c4-c4ccc(-c5ccccc5)cc4)cc3)c3cccc(-c4ccccc4)c3)cc21]

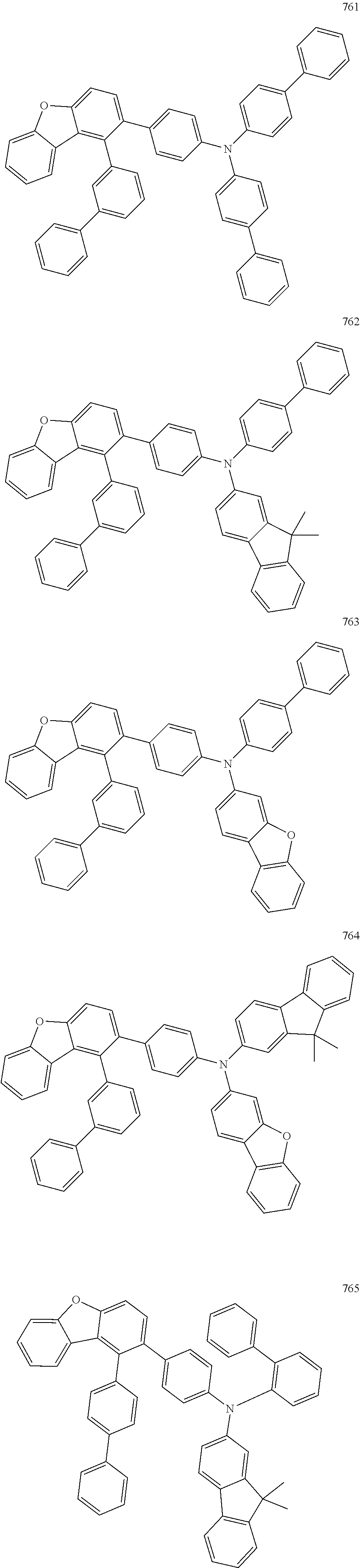 [SMILES: c1ccc(-c2ccc(N(c3ccc(-c4ccccc4)cc3)c3ccc(-c4ccc5oc6ccccc6c5c4-c4cccc(-c5ccccc5)c4)cc3)cc2)cc1 ; CC1(C)c2ccccc2-c2ccc(N(c3ccc(-c4ccccc4)cc3)c3ccc(-c4ccc5oc6ccccc6c5c4-c4cccc(-c5ccccc5)c4)cc3)cc21 ; c1ccc(-c2ccc(N(c3ccc(-c4ccc5oc6ccccc6c5c4-c4cccc(-c5ccccc5)c4)cc3)c3ccc4c(c3)oc3ccccc34)cc2)cc1 ; CC1(C)c2ccccc2-c2ccc(N(c3ccc(-c4ccc5oc6ccccc6c5c4-c4cccc(-c5ccccc5)c4)cc3)c3ccc4c(c3)oc3ccccc34)cc21 ; CC1(C)c2ccccc2-c2ccc(N(c3ccc(-c4ccc5oc6ccccc6c5c4-c4ccc(-c5ccccc5)cc4)cc3)c3ccccc3-c3ccccc3)cc21]

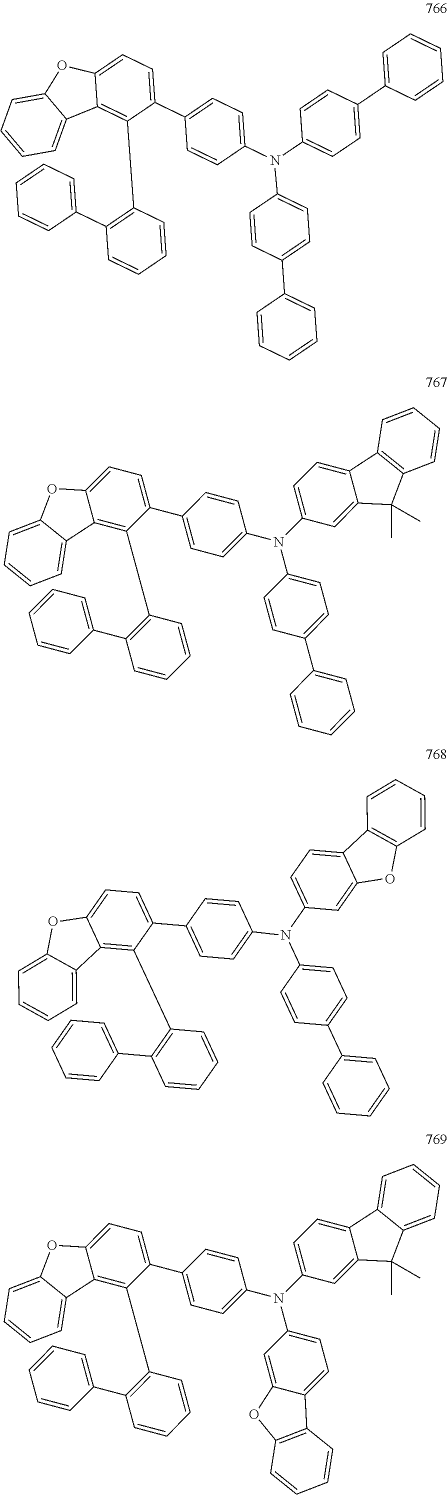 [SMILES: c1ccc(-c2ccc(N(c3ccc(-c4ccccc4)cc3)c3ccc(-c4ccc5oc6ccccc6c5c4-c4ccccc4-c4ccccc4)cc3)cc2)cc1 ; CC1(C)c2ccccc2-c2ccc(N(c3ccc(-c4ccccc4)cc3)c3ccc(-c4ccc5oc6ccccc6c5c4-c4ccccc4-c4ccccc4)cc3)cc21 ; c1ccc(-c2ccc(N(c3ccc(-c4ccc5oc6ccccc6c5c4-c4ccccc4-c4ccccc4)cc3)c3ccc4c(c3)oc3ccccc34)cc2)cc1 ; CC1(C)c2ccccc2-c2ccc(N(c3ccc(-c4ccc5oc6ccccc6c5c4-c4ccccc4-c4ccccc4)cc3)c3ccc4c(c3)oc3ccccc34)cc21]

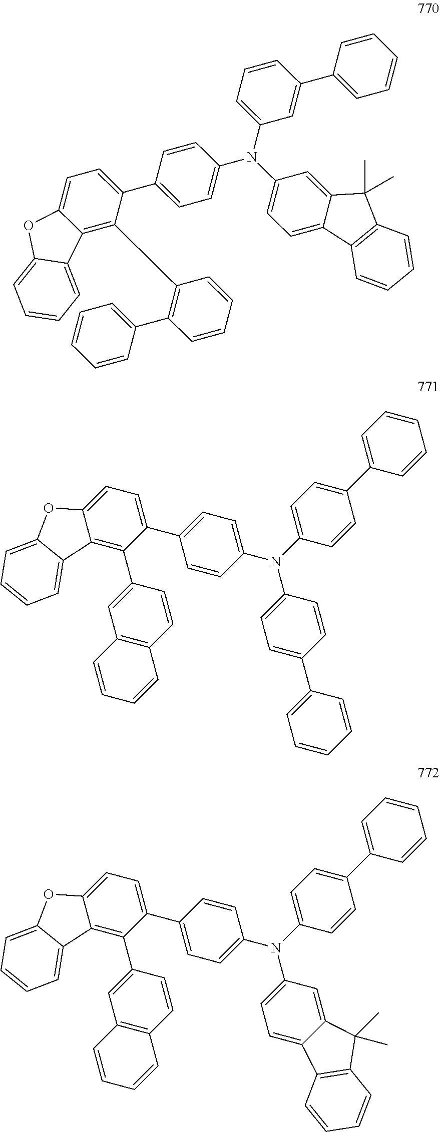 [SMILES: CC1(C)c2ccccc2-c2ccc(N(c3ccc(-c4ccc5oc6ccccc6c5c4-c4ccccc4-c4ccccc4)cc3)c3cccc(-c4ccccc4)c3)cc21 ; c1ccc(-c2ccc(N(c3ccc(-c4ccccc4)cc3)c3ccc(-c4ccc5oc6ccccc6c5c4-c4ccc5ccccc5c4)cc3)cc2)cc1 ; CC1(C)c2ccccc2-c2ccc(N(c3ccc(-c4ccccc4)cc3)c3ccc(-c4ccc5oc6ccccc6c5c4-c4ccc5ccccc5c4)cc3)cc21]

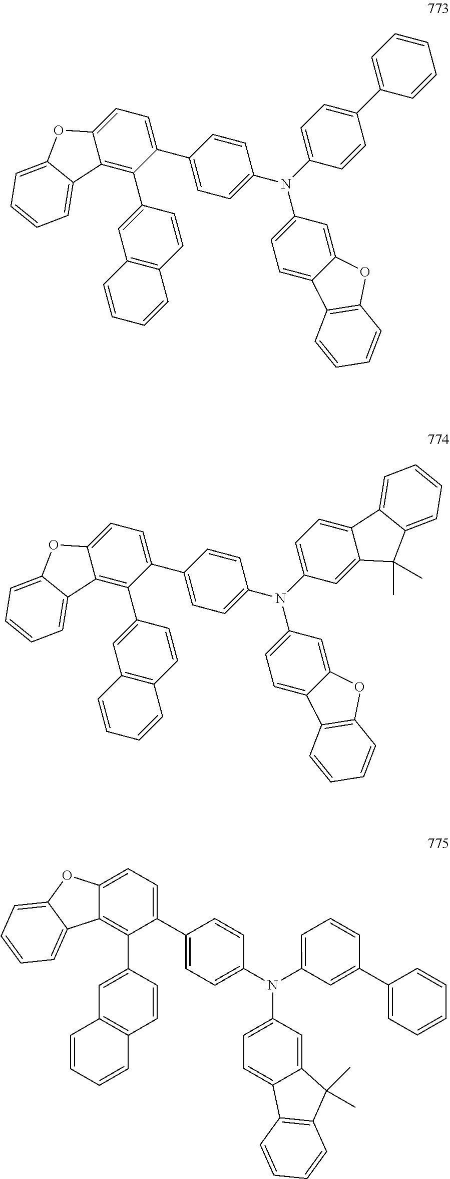 [SMILES: c1ccc(-c2ccc(N(c3ccc(-c4ccc5oc6ccccc6c5c4-c4ccc5ccccc5c4)cc3)c3ccc4c(c3)oc3ccccc34)cc2)cc1 ; CC1(C)c2ccccc2-c2ccc(N(c3ccc(-c4ccc5oc6ccccc6c5c4-c4ccc5ccccc5c4)cc3)c3ccc4c(c3)oc3ccccc34)cc21 ; CC1(C)c2ccccc2-c2ccc(N(c3ccc(-c4ccc5oc6ccccc6c5c4-c4ccc5ccccc5c4)cc3)c3cccc(-c4ccccc4)c3)cc21]

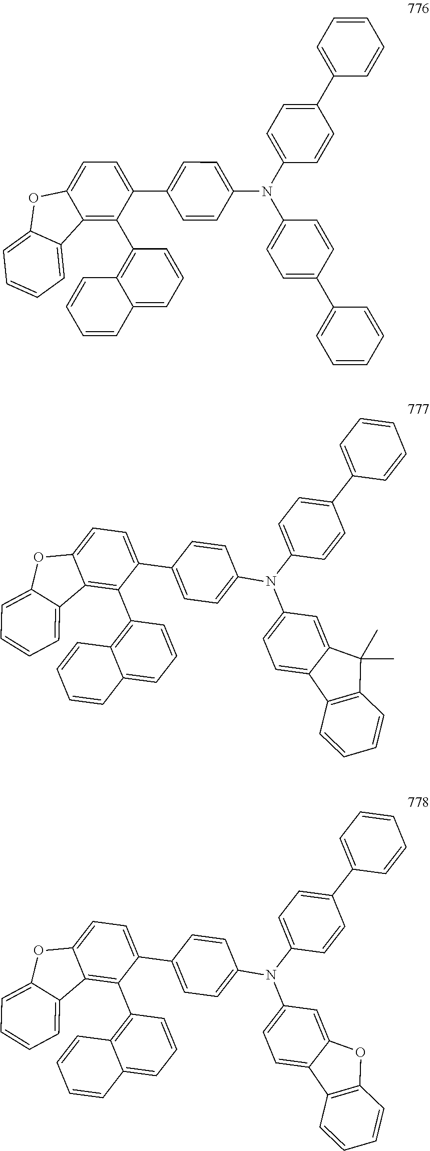 [SMILES: c1ccc(-c2ccc(N(c3ccc(-c4ccccc4)cc3)c3ccc(-c4ccc5oc6ccccc6c5c4-c4cccc5ccccc45)cc3)cc2)cc1 ; CC1(C)c2ccccc2-c2ccc(N(c3ccc(-c4ccccc4)cc3)c3ccc(-c4ccc5oc6ccccc6c5c4-c4cccc5ccccc45)cc3)cc21 ; c1ccc(-c2ccc(N(c3ccc(-c4ccc5oc6ccccc6c5c4-c4cccc5ccccc45)cc3)c3ccc4c(c3)oc3ccccc34)cc2)cc1]

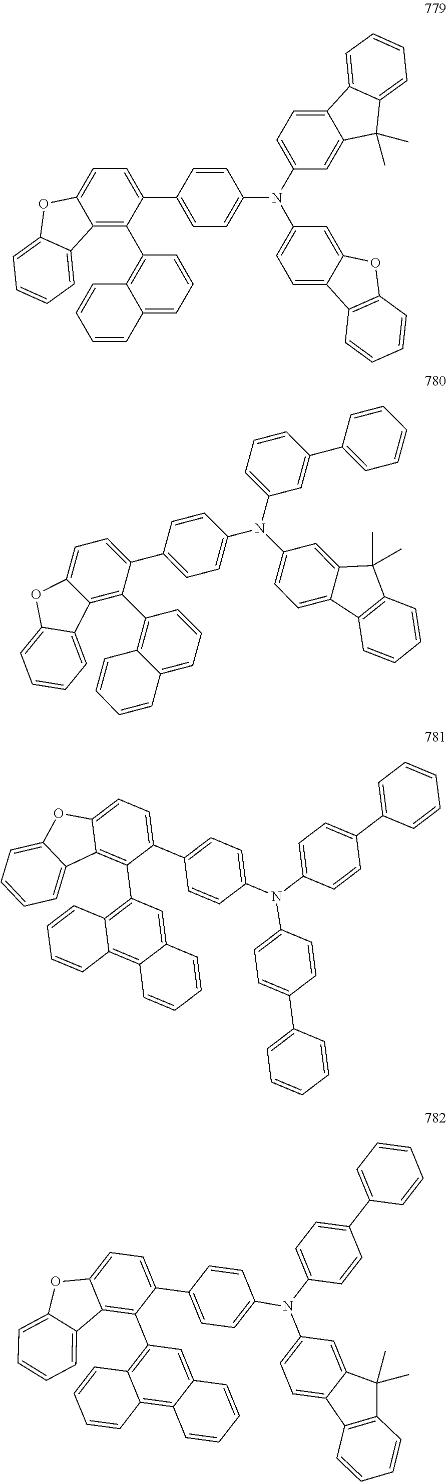

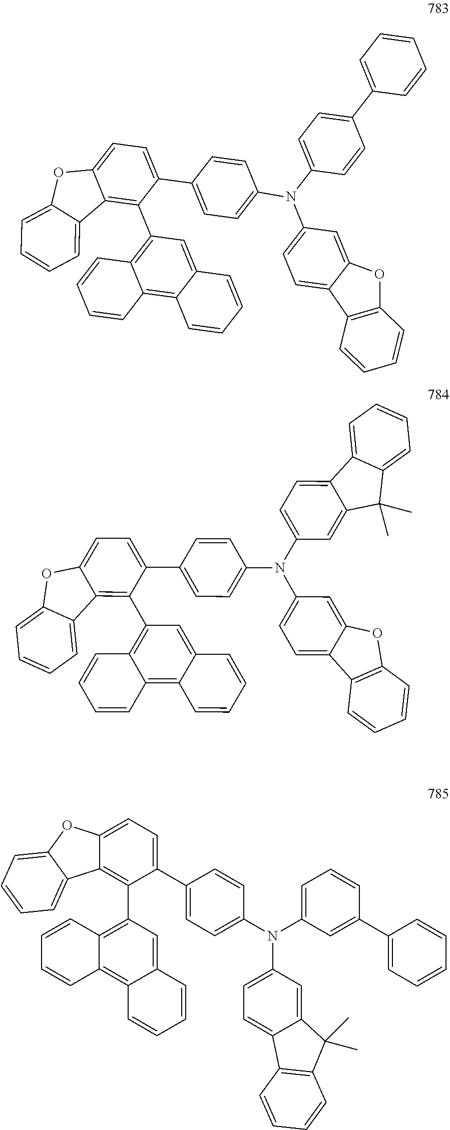

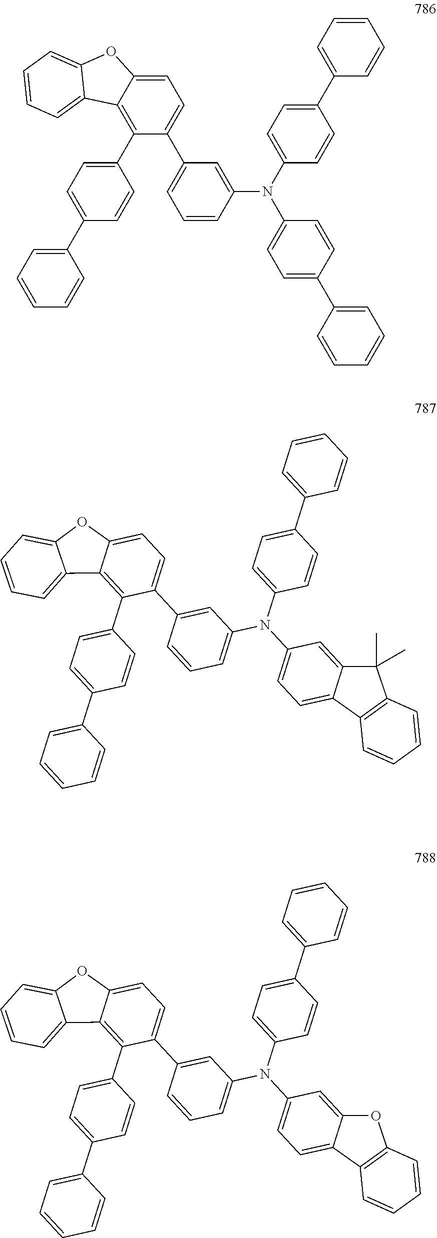 [SMILES: c1ccc(-c2ccc(-c3c(-c4cccc(N(c5ccc(-c6ccccc6)cc5)c5ccc(-c6ccccc6)cc5)c4)ccc4oc5ccccc5c34)cc2)cc1 ; CC1(C)c2ccccc2-c2ccc(N(c3ccc(-c4ccccc4)cc3)c3cccc(-c4ccc5oc6ccccc6c5c4-c4ccc(-c5ccccc5)cc4)c3)cc21 ; c1ccc(-c2ccc(-c3c(-c4cccc(N(c5ccc(-c6ccccc6)cc5)c5ccc6c(c5)oc5ccccc56)c4)ccc4oc5ccccc5c34)cc2)cc1]

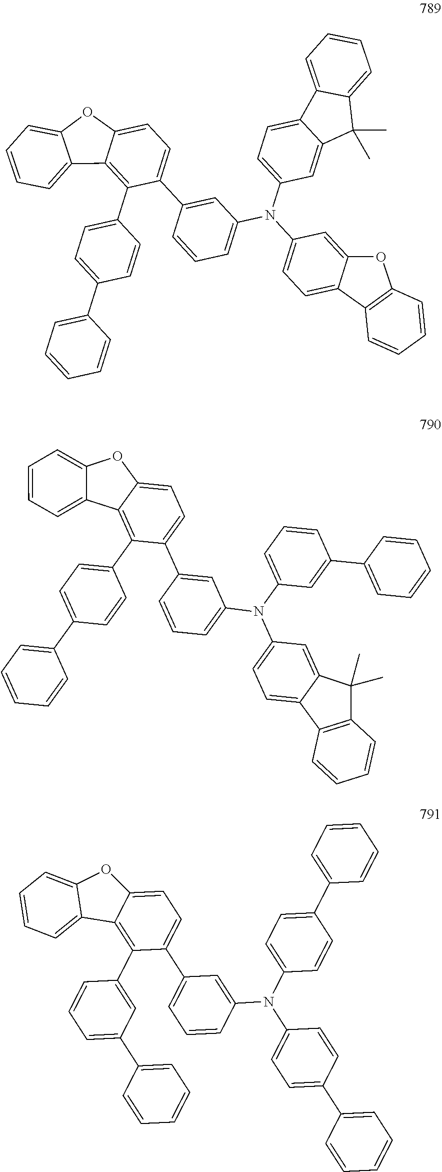

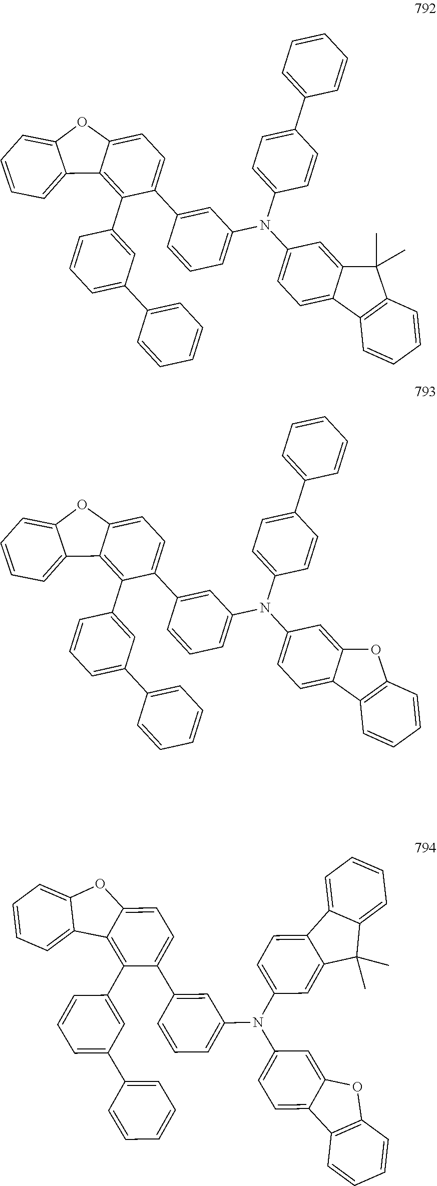 [SMILES: CC1(C)c2ccccc2-c2ccc(N(c3ccc(-c4ccccc4)cc3)c3cccc(-c4ccc5oc6ccccc6c5c4-c4cccc(-c5ccccc5)c4)c3)cc21 ; c1ccc(-c2ccc(N(c3cccc(-c4ccc5oc6ccccc6c5c4-c4cccc(-c5ccccc5)c4)c3)c3ccc4c(c3)oc3ccccc34)cc2)cc1 ; CC1(C)c2ccccc2-c2ccc(N(c3cccc(-c4ccc5oc6ccccc6c5c4-c4cccc(-c5ccccc5)c4)c3)c3ccc4c(c3)oc3ccccc34)cc21]

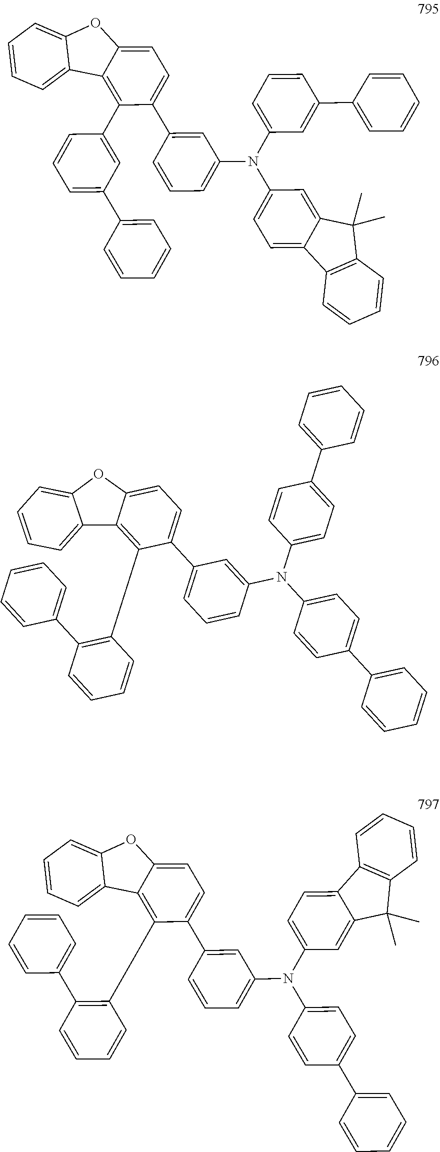 [SMILES: CC1(C)c2ccccc2-c2ccc(N(c3cccc(-c4ccccc4)c3)c3cccc(-c4ccc5oc6ccccc6c5c4-c4cccc(-c5ccccc5)c4)c3)cc21 ; c1ccc(-c2ccc(N(c3ccc(-c4ccccc4)cc3)c3cccc(-c4ccc5oc6ccccc6c5c4-c4ccccc4-c4ccccc4)c3)cc2)cc1 ; CC1(C)c2ccccc2-c2ccc(N(c3ccc(-c4ccccc4)cc3)c3cccc(-c4ccc5oc6ccccc6c5c4-c4ccccc4-c4ccccc4)c3)cc21]

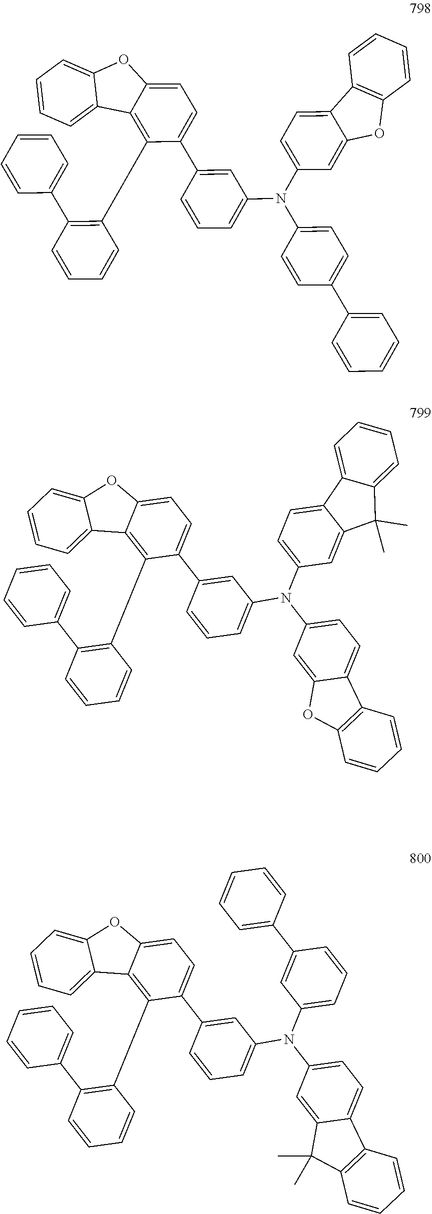 [SMILES: c1ccc(-c2ccc(N(c3cccc(-c4ccc5oc6ccccc6c5c4-c4ccccc4-c4ccccc4)c3)c3ccc4c(c3)oc3ccccc34)cc2)cc1 ; CC1(C)c2ccccc2-c2ccc(N(c3cccc(-c4ccc5oc6ccccc6c5c4-c4ccccc4-c4ccccc4)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4ccccc4-c4ccccc4)c3)cc21]

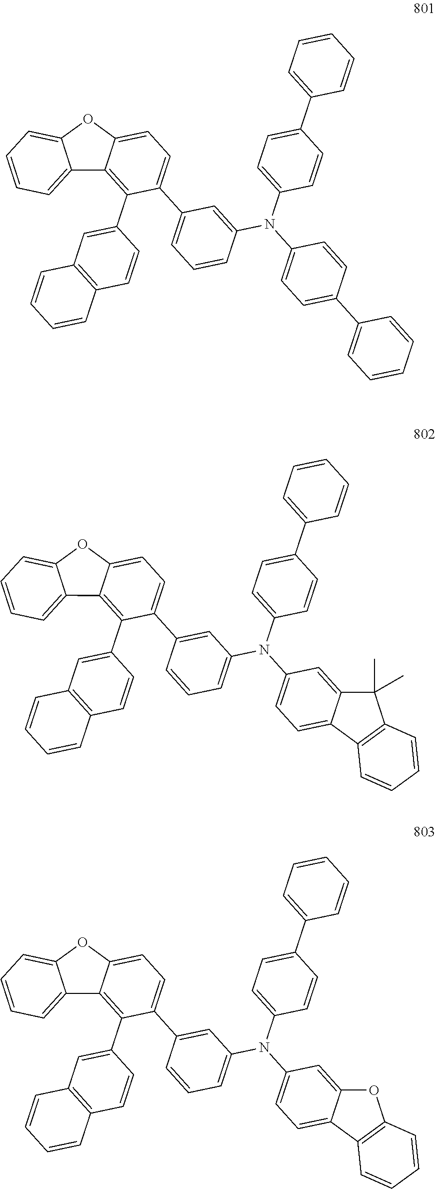 [SMILES: c1ccc(-c2ccc(N(c3ccc(-c4ccccc4)cc3)c3cccc(-c4ccc5oc6ccccc6c5c4-c4ccc5ccccc5c4)c3)cc2)cc1 ; CC1(C)c2ccccc2-c2ccc(N(c3ccc(-c4ccccc4)cc3)c3cccc(-c4ccc5oc6ccccc6c5c4-c4ccc5ccccc5c4)c3)cc21 ; c1ccc(-c2ccc(N(c3cccc(-c4ccc5oc6ccccc6c5c4-c4ccc5ccccc5c4)c3)c3ccc4c(c3)oc3ccccc34)cc2)cc1]

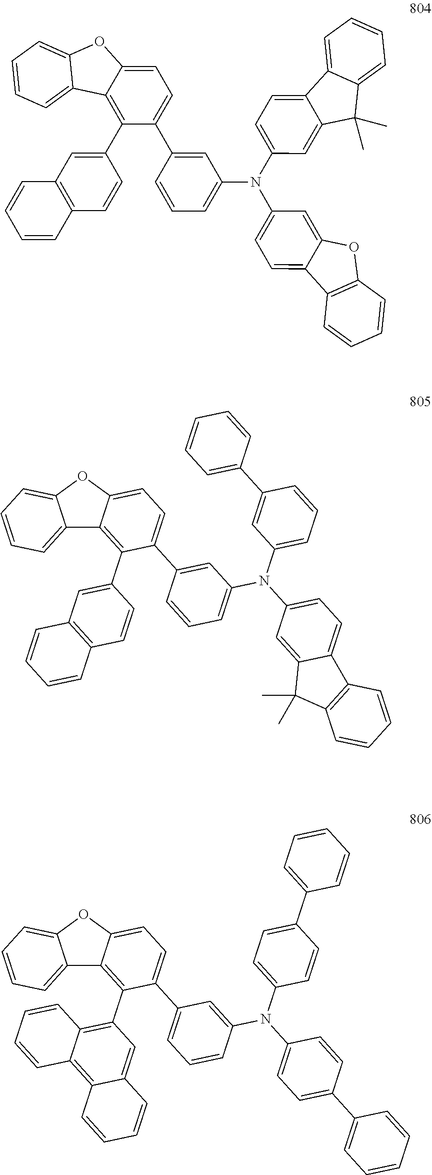 [SMILES: CC1(C)c2ccccc2-c2ccc(N(c3cccc(-c4ccc5oc6ccccc6c5c4-c4ccc5ccccc5c4)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4ccc5ccccc5c4)c3)cc21 ; c1ccc(-c2ccc(N(c3ccc(-c4ccccc4)cc3)c3cccc(-c4ccc5oc6ccccc6c5c4-c4cc5ccccc5c5ccccc45)c3)cc2)cc1]

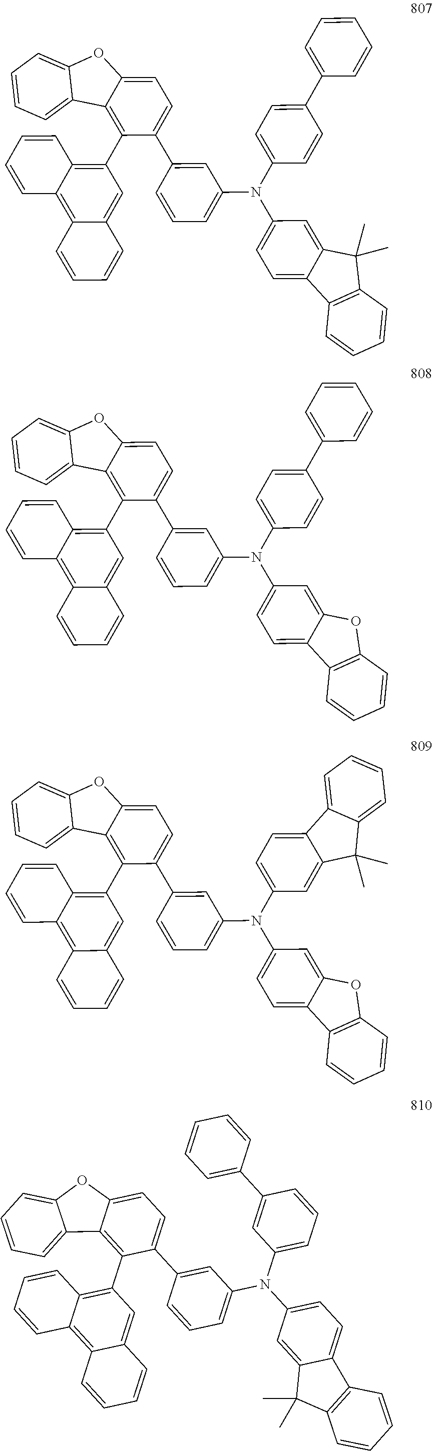

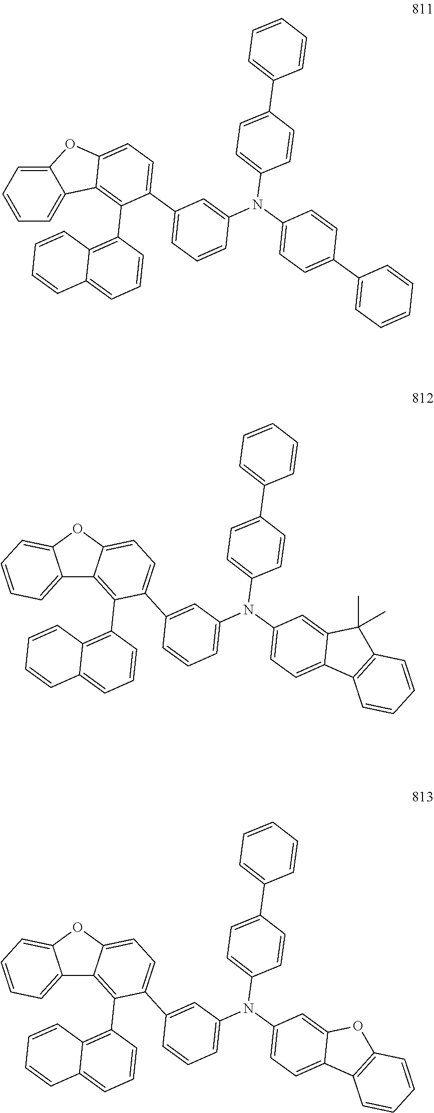 [SMILES: c1ccc(-c2ccc(N(c3ccc(-c4ccccc4)cc3)c3cccc(-c4ccc5oc6ccccc6c5c4-c4cccc5ccccc45)c3)cc2)cc1 ; CC1(C)c2ccccc2-c2ccc(N(c3ccc(-c4ccccc4)cc3)c3cccc(-c4ccc5oc6ccccc6c5c4-c4cccc5ccccc45)c3)cc21 ; c1ccc(-c2ccc(N(c3cccc(-c4ccc5oc6ccccc6c5c4-c4cccc5ccccc45)c3)c3ccc4c(c3)oc3ccccc34)cc2)cc1]

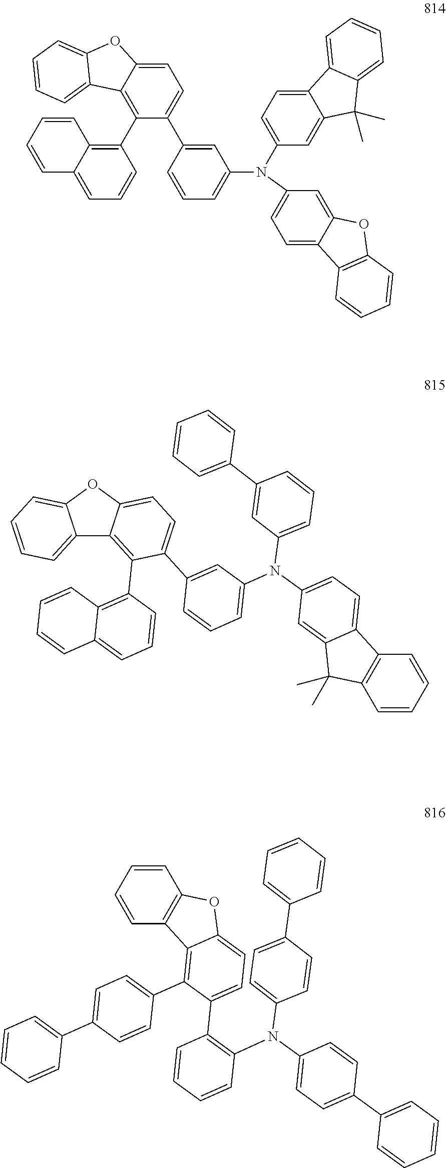 [SMILES: CC1(C)c2ccccc2-c2ccc(N(c3cccc(-c4ccc5oc6ccccc6c5c4-c4cccc5ccccc45)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4cccc5ccccc45)c3)cc21 ; c1ccc(-c2ccc(-c3c(-c4ccccc4N(c4ccc(-c5ccccc5)cc4)c4ccc(-c5ccccc5)cc4)ccc4oc5ccccc5c34)cc2)cc1]

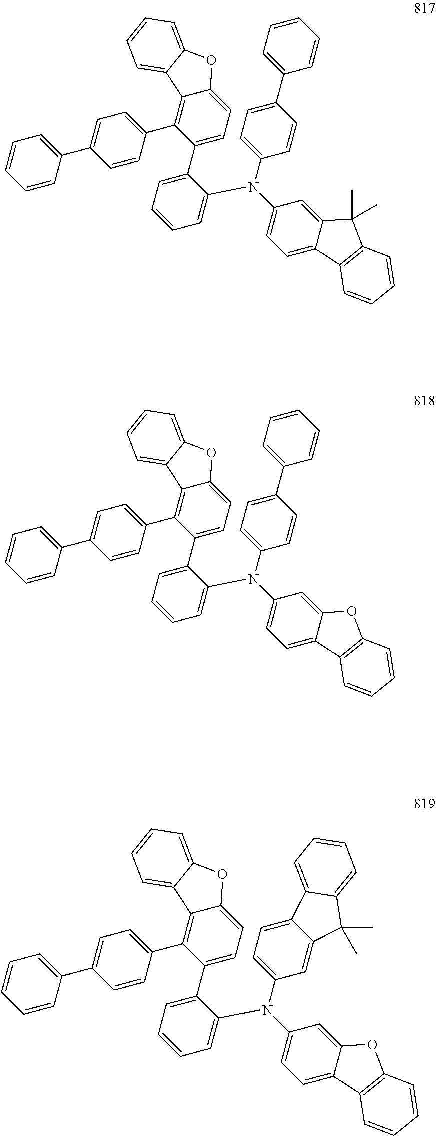 [SMILES: CC1(C)c2ccccc2-c2ccc(N(c3ccc(-c4ccccc4)cc3)c3ccccc3-c3ccc4oc5ccccc5c4c3-c3ccc(-c4ccccc4)cc3)cc21 ; c1ccc(-c2ccc(-c3c(-c4ccccc4N(c4ccc(-c5ccccc5)cc4)c4ccc5c(c4)oc4ccccc45)ccc4oc5ccccc5c34)cc2)cc1 ; CC1(C)c2ccccc2-c2ccc(N(c3ccc4c(c3)oc3ccccc34)c3ccccc3-c3ccc4oc5ccccc5c4c3-c3ccc(-c4ccccc4)cc3)cc21]

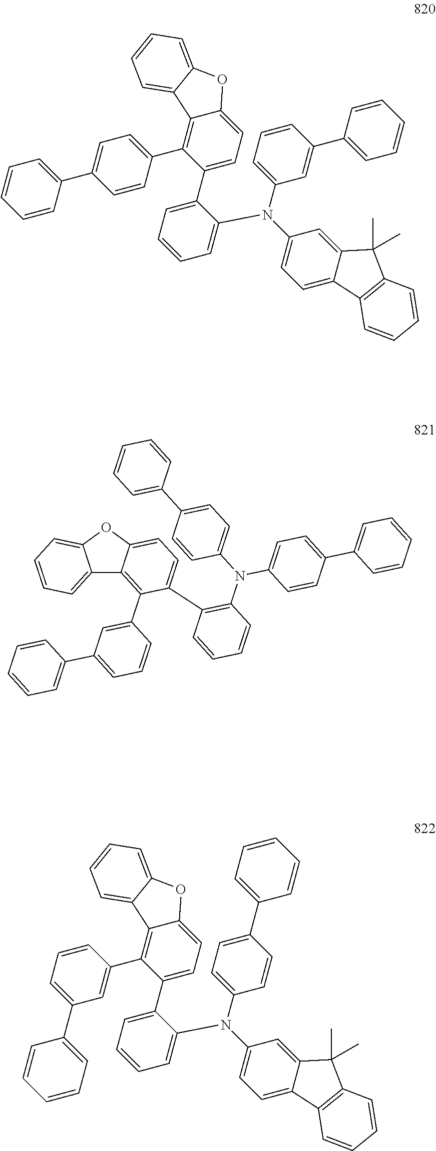 [SMILES: CC1(C)c2ccccc2-c2ccc(N(c3cccc(-c4ccccc4)c3)c3ccccc3-c3ccc4oc5ccccc5c4c3-c3ccc(-c4ccccc4)cc3)cc21 ; c1ccc(-c2ccc(N(c3ccc(-c4ccccc4)cc3)c3ccccc3-c3ccc4oc5ccccc5c4c3-c3cccc(-c4ccccc4)c3)cc2)cc1 ; CC1(C)c2ccccc2-c2ccc(N(c3ccc(-c4ccccc4)cc3)c3ccccc3-c3ccc4oc5ccccc5c4c3-c3cccc(-c4ccccc4)c3)cc21]

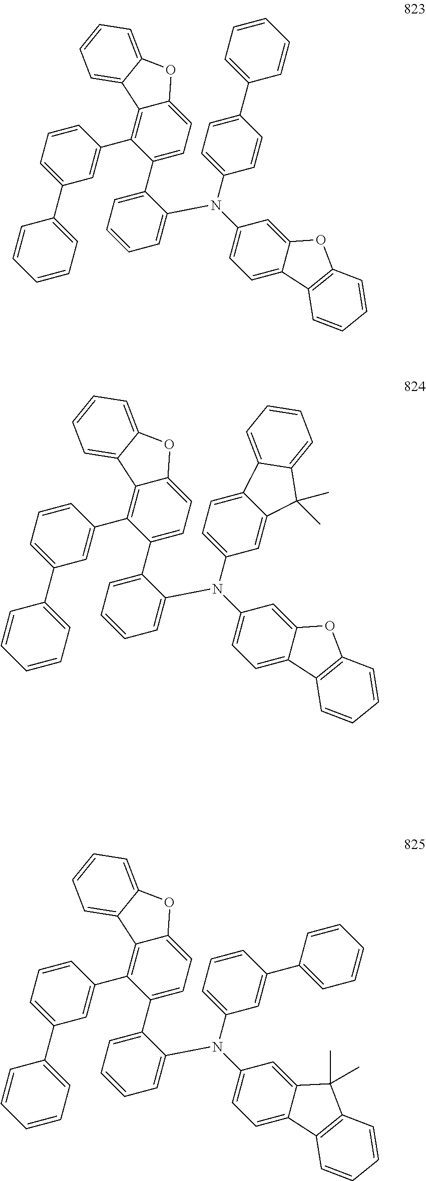 [SMILES: c1ccc(-c2ccc(N(c3ccc4c(c3)oc3ccccc34)c3ccccc3-c3ccc4oc5ccccc5c4c3-c3cccc(-c4ccccc4)c3)cc2)cc1 ; CC1(C)c2ccccc2-c2ccc(N(c3ccc4c(c3)oc3ccccc34)c3ccccc3-c3ccc4oc5ccccc5c4c3-c3cccc(-c4ccccc4)c3)cc21 ; CC1(C)c2ccccc2-c2ccc(N(c3cccc(-c4ccccc4)c3)c3ccccc3-c3ccc4oc5ccccc5c4c3-c3cccc(-c4ccccc4)c3)cc21]

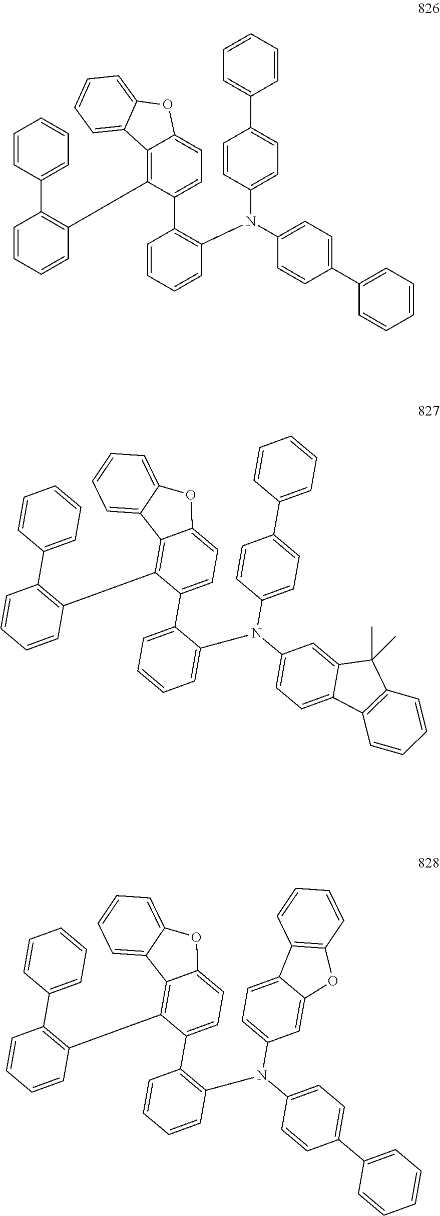 [SMILES: c1ccc(-c2ccc(N(c3ccc(-c4ccccc4)cc3)c3ccccc3-c3ccc4oc5ccccc5c4c3-c3ccccc3-c3ccccc3)cc2)cc1 ; CC1(C)c2ccccc2-c2ccc(N(c3ccc(-c4ccccc4)cc3)c3ccccc3-c3ccc4oc5ccccc5c4c3-c3ccccc3-c3ccccc3)cc21 ; c1ccc(-c2ccc(N(c3ccc4c(c3)oc3ccccc34)c3ccccc3-c3ccc4oc5ccccc5c4c3-c3ccccc3-c3ccccc3)cc2)cc1]

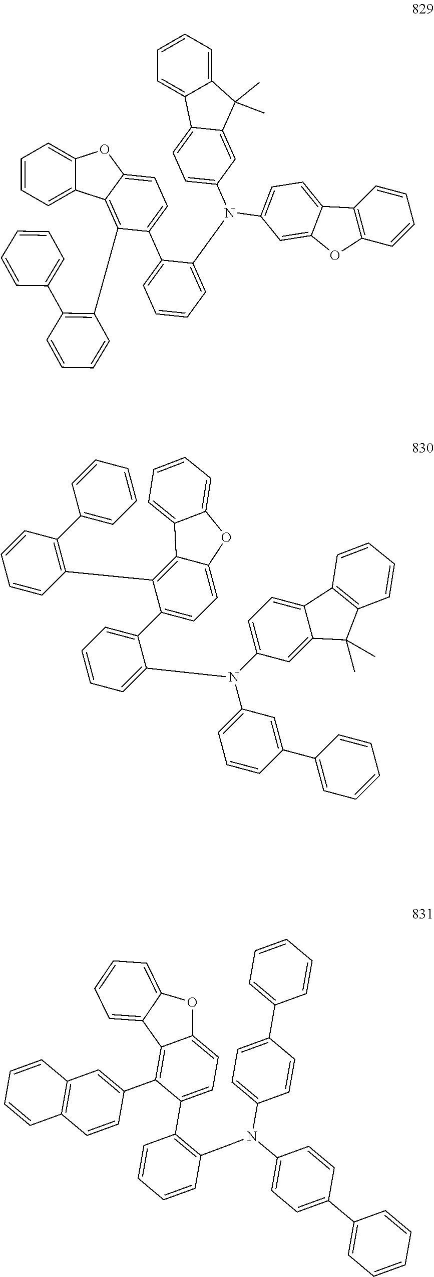 [SMILES: CC1(C)c2ccccc2-c2ccc(N(c3ccc4c(c3)oc3ccccc34)c3ccccc3-c3ccc4oc5ccccc5c4c3-c3ccccc3-c3ccccc3)cc21 ; CC1(C)c2ccccc2-c2ccc(N(c3cccc(-c4ccccc4)c3)c3ccccc3-c3ccc4oc5ccccc5c4c3-c3ccccc3-c3ccccc3)cc21 ; c1ccc(-c2ccc(N(c3ccc(-c4ccccc4)cc3)c3ccccc3-c3ccc4oc5ccccc5c4c3-c3ccc4ccccc4c3)cc2)cc1]

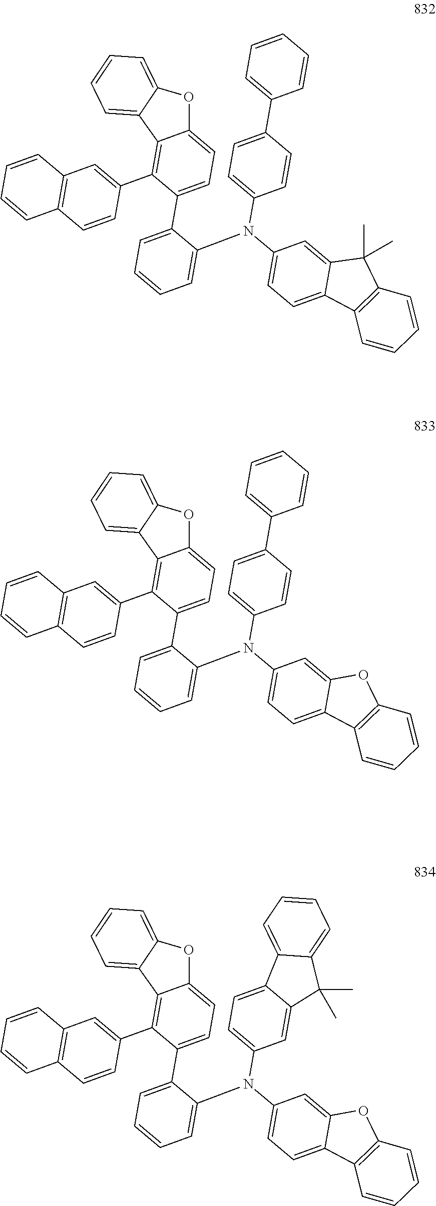 [SMILES: CC1(C)c2ccccc2-c2ccc(N(c3ccc(-c4ccccc4)cc3)c3ccccc3-c3ccc4oc5ccccc5c4c3-c3ccc4ccccc4c3)cc21 ; c1ccc(-c2ccc(N(c3ccc4c(c3)oc3ccccc34)c3ccccc3-c3ccc4oc5ccccc5c4c3-c3ccc4ccccc4c3)cc2)cc1 ; CC1(C)c2ccccc2-c2ccc(N(c3ccc4c(c3)oc3ccccc34)c3ccccc3-c3ccc4oc5ccccc5c4c3-c3ccc4ccccc4c3)cc21]

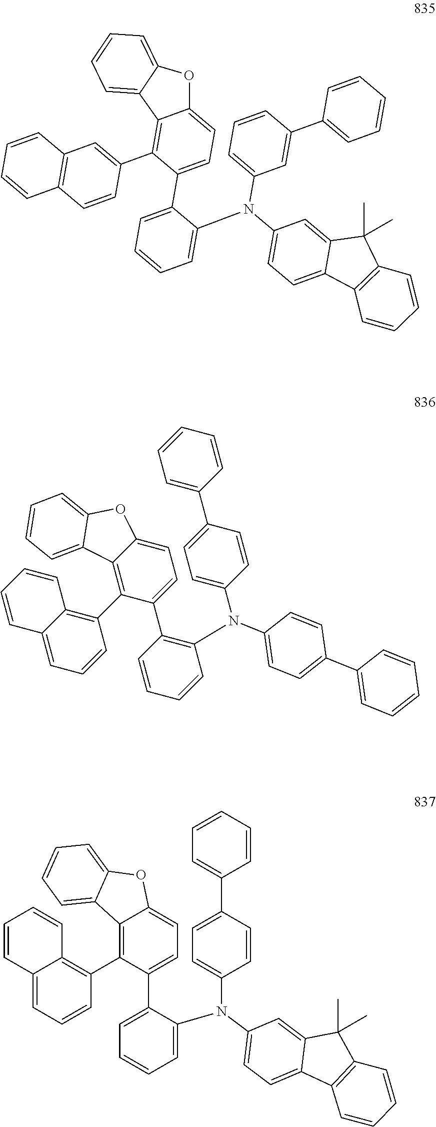 [SMILES: CC1(C)c2ccccc2-c2ccc(N(c3cccc(-c4ccccc4)c3)c3ccccc3-c3ccc4oc5ccccc5c4c3-c3ccc4ccccc4c3)cc21 ; c1ccc(-c2ccc(N(c3ccc(-c4ccccc4)cc3)c3ccccc3-c3ccc4oc5ccccc5c4c3-c3cccc4ccccc34)cc2)cc1 ; CC1(C)c2ccccc2-c2ccc(N(c3ccc(-c4ccccc4)cc3)c3ccccc3-c3ccc4oc5ccccc5c4c3-c3cccc4ccccc34)cc21]

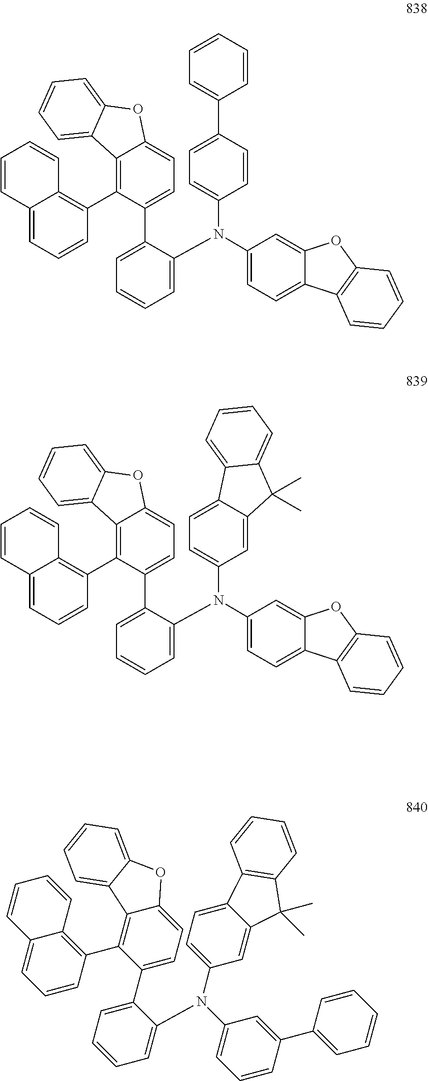 [SMILES: c1ccc(-c2ccc(N(c3ccc4c(c3)oc3ccccc34)c3ccccc3-c3ccc4oc5ccccc5c4c3-c3cccc4ccccc34)cc2)cc1 ; CC1(C)c2ccccc2-c2ccc(N(c3ccc4c(c3)oc3ccccc34)c3ccccc3-c3ccc4oc5ccccc5c4c3-c3cccc4ccccc34)cc21 ; CC1(C)c2ccccc2-c2ccc(N(c3cccc(-c4ccccc4)c3)c3ccccc3-c3ccc4oc5ccccc5c4c3-c3cccc4ccccc34)cc21]

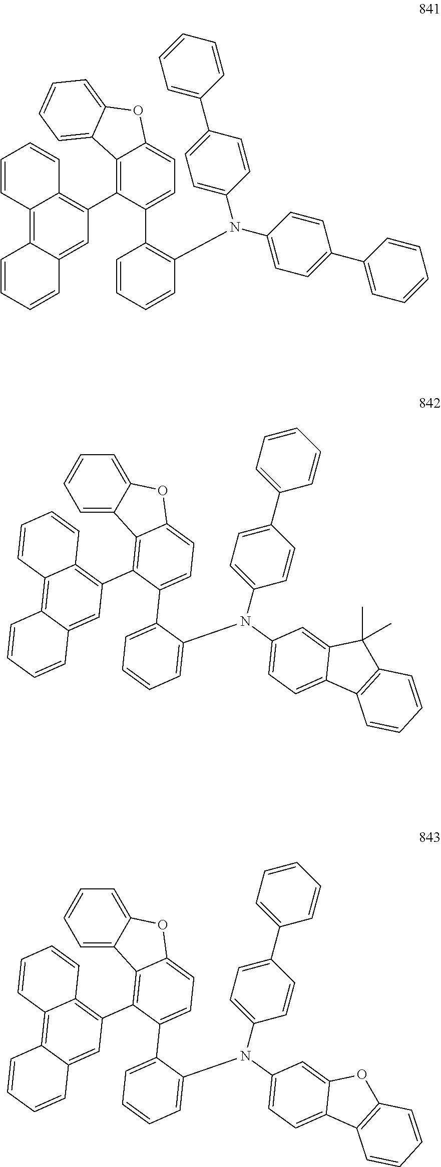 [SMILES: c1ccc(-c2ccc(N(c3ccc(-c4ccccc4)cc3)c3ccccc3-c3ccc4oc5ccccc5c4c3-c3cc4ccccc4c4ccccc34)cc2)cc1 ; CC1(C)c2ccccc2-c2ccc(N(c3ccc(-c4ccccc4)cc3)c3ccccc3-c3ccc4oc5ccccc5c4c3-c3cc4ccccc4c4ccccc34)cc21 ; c1ccc(-c2ccc(N(c3ccc4c(c3)oc3ccccc34)c3ccccc3-c3ccc4oc5ccccc5c4c3-c3cc4ccccc4c4ccccc34)cc2)cc1]

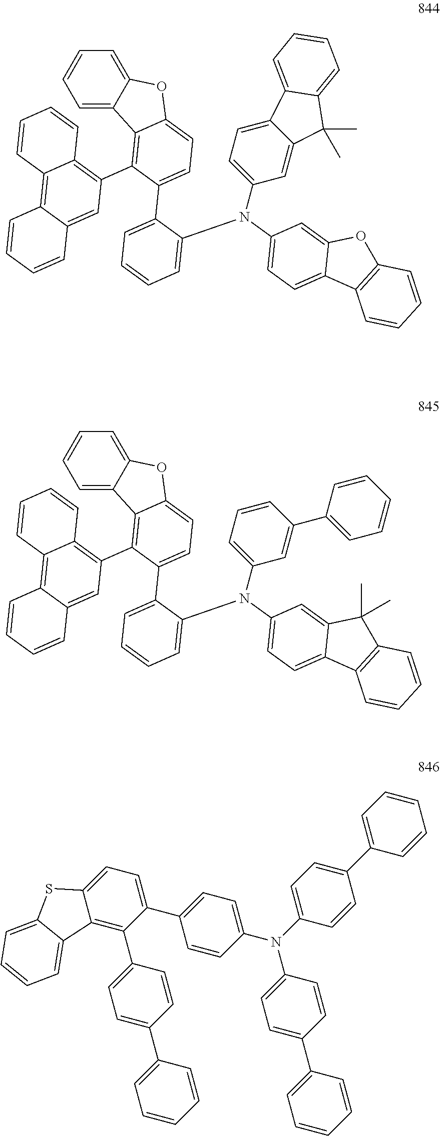 [SMILES: CC1(C)c2ccccc2-c2ccc(N(c3ccc4c(c3)oc3ccccc34)c3ccccc3-c3ccc4oc5ccccc5c4c3-c3cc4ccccc4c4ccccc34)cc21 ; CC1(C)c2ccccc2-c2ccc(N(c3cccc(-c4ccccc4)c3)c3ccccc3-c3ccc4oc5ccccc5c4c3-c3cc4ccccc4c4ccccc34)cc21 ; c1ccc(-c2ccc(-c3c(-c4ccc(N(c5ccc(-c6ccccc6)cc5)c5ccc(-c6ccccc6)cc5)cc4)ccc4sc5ccccc5c34)cc2)cc1]

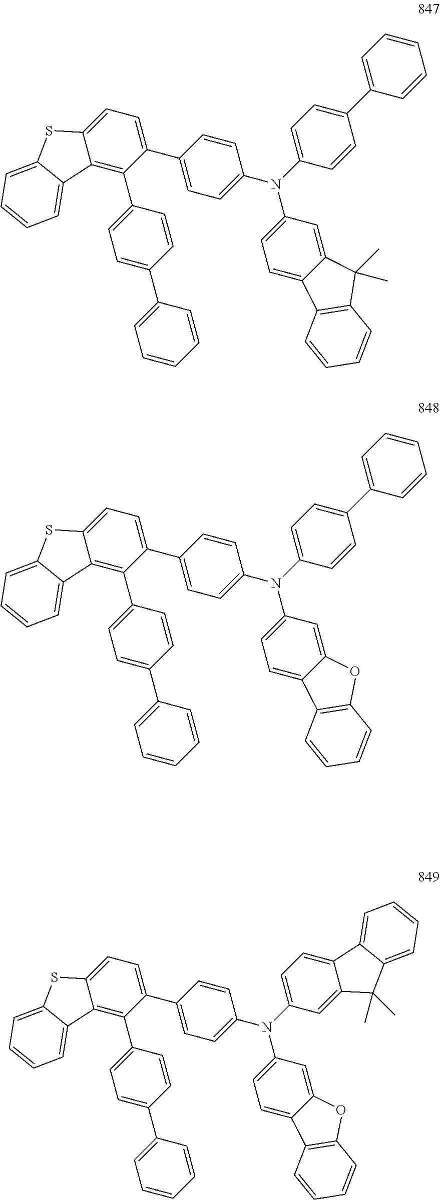 [SMILES: CC1(C)c2ccccc2-c2ccc(N(c3ccc(-c4ccccc4)cc3)c3ccc(-c4ccc5sc6ccccc6c5c4-c4ccc(-c5ccccc5)cc4)cc3)cc21 ; c1ccc(-c2ccc(-c3c(-c4ccc(N(c5ccc(-c6ccccc6)cc5)c5ccc6c(c5)oc5ccccc56)cc4)ccc4sc5ccccc5c34)cc2)cc1 ; CC1(C)c2ccccc2-c2ccc(N(c3ccc(-c4ccc5sc6ccccc6c5c4-c4ccc(-c5ccccc5)cc4)cc3)c3ccc4c(c3)oc3ccccc34)cc21]

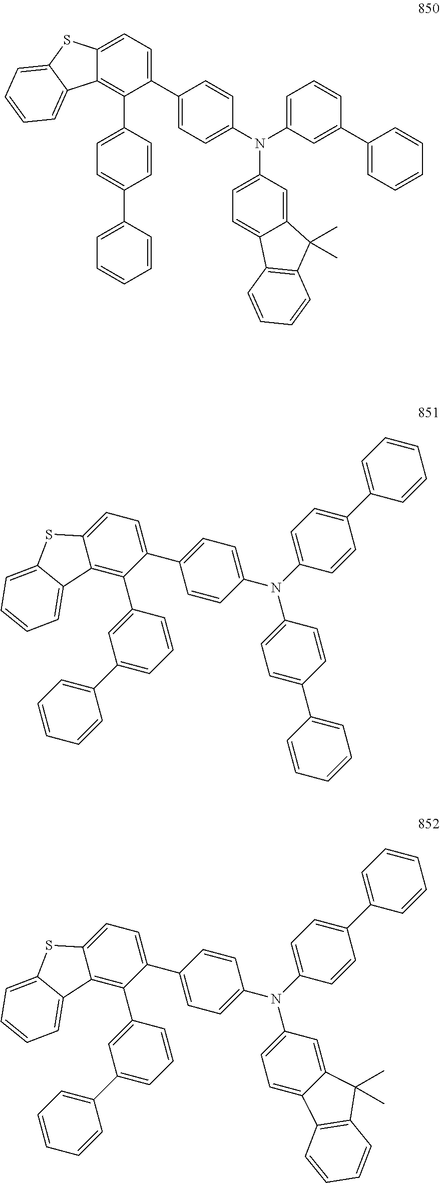 [SMILES: CC1(C)c2ccccc2-c2ccc(N(c3ccc(-c4ccc5sc6ccccc6c5c4-c4ccc(-c5ccccc5)cc4)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4cccc(-c5ccccc5)c4)cc3)cc2)cc1 ; CC1(C)c2ccccc2-c2ccc(N(c3ccc(-c4ccccc4)cc3)c3ccc(-c4ccc5sc6ccccc6c5c4-c4cccc(-c5ccccc5)c4)cc3)cc21]

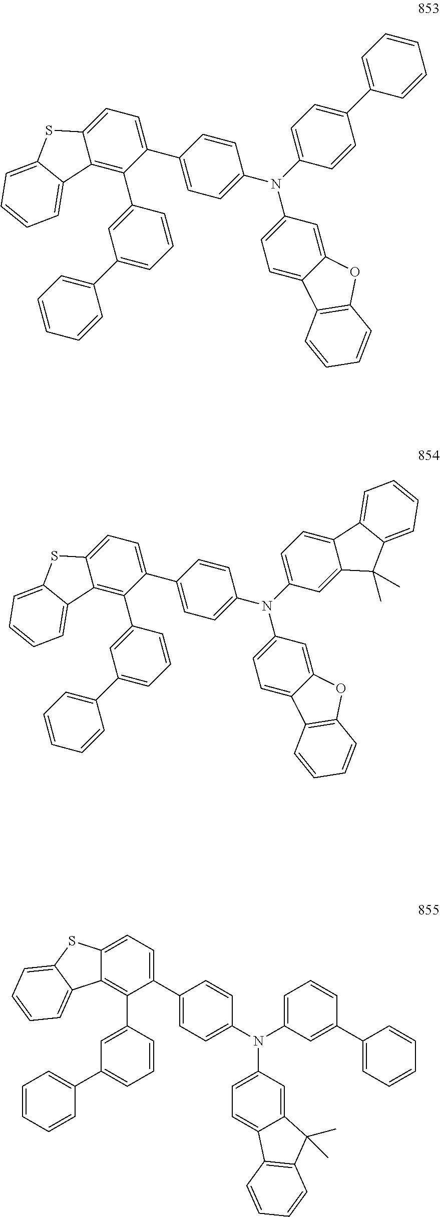 [SMILES: c1ccc(-c2ccc(N(c3ccc(-c4ccc5sc6ccccc6c5c4-c4cccc(-c5ccccc5)c4)cc3)c3ccc4c(c3)oc3ccccc34)cc2)cc1 ; CC1(C)c2ccccc2-c2ccc(N(c3ccc(-c4ccc5sc6ccccc6c5c4-c4cccc(-c5ccccc5)c4)cc3)c3ccc4c(c3)oc3ccccc34)cc21 ; CC1(C)c2ccccc2-c2ccc(N(c3ccc(-c4ccc5sc6ccccc6c5c4-c4cccc(-c5ccccc5)c4)cc3)c3cccc(-c4ccccc4)c3)cc21]

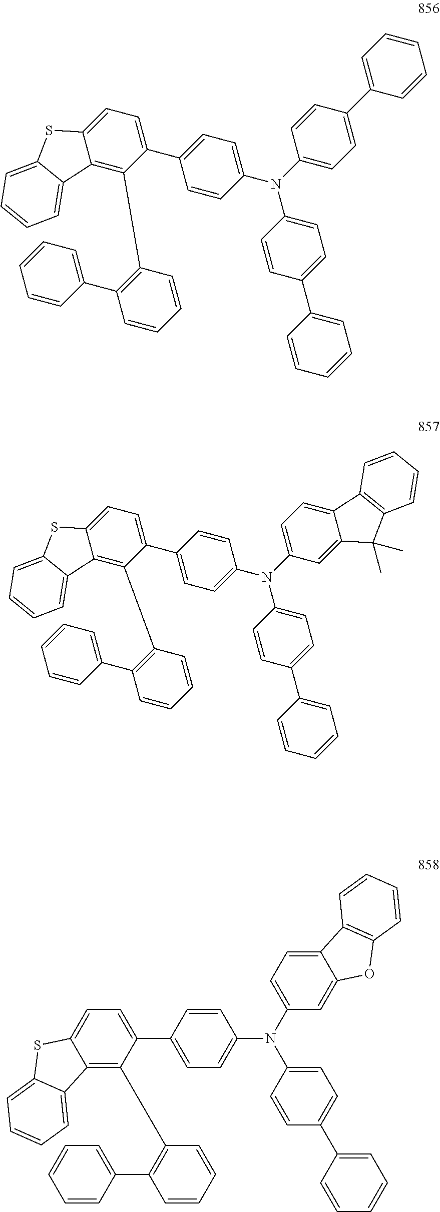 [SMILES: c1ccc(-c2ccc(N(c3ccc(-c4ccccc4)cc3)c3ccc(-c4ccc5sc6ccccc6c5c4-c4ccccc4-c4ccccc4)cc3)cc2)cc1 ; CC1(C)c2ccccc2-c2ccc(N(c3ccc(-c4ccccc4)cc3)c3ccc(-c4ccc5sc6ccccc6c5c4-c4ccccc4-c4ccccc4)cc3)cc21 ; c1ccc(-c2ccc(N(c3ccc(-c4ccc5sc6ccccc6c5c4-c4ccccc4-c4ccccc4)cc3)c3ccc4c(c3)oc3ccccc34)cc2)cc1]

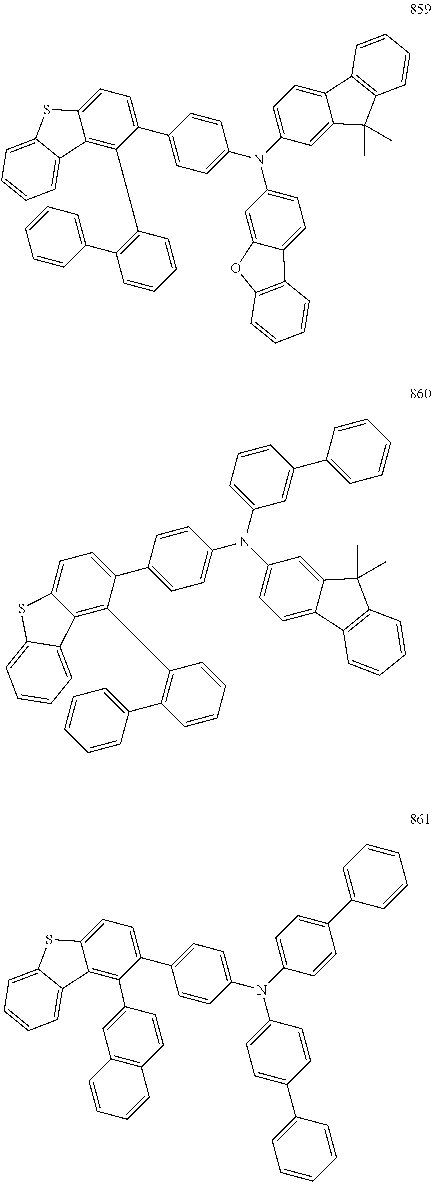 [SMILES: CC1(C)c2ccccc2-c2ccc(N(c3ccc(-c4ccc5sc6ccccc6c5c4-c4ccccc4-c4ccccc4)cc3)c3ccc4c(c3)oc3ccccc34)cc21 ; CC1(C)c2ccccc2-c2ccc(N(c3ccc(-c4ccc5sc6ccccc6c5c4-c4ccccc4-c4ccccc4)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4ccc5ccccc5c4)cc3)cc2)cc1]

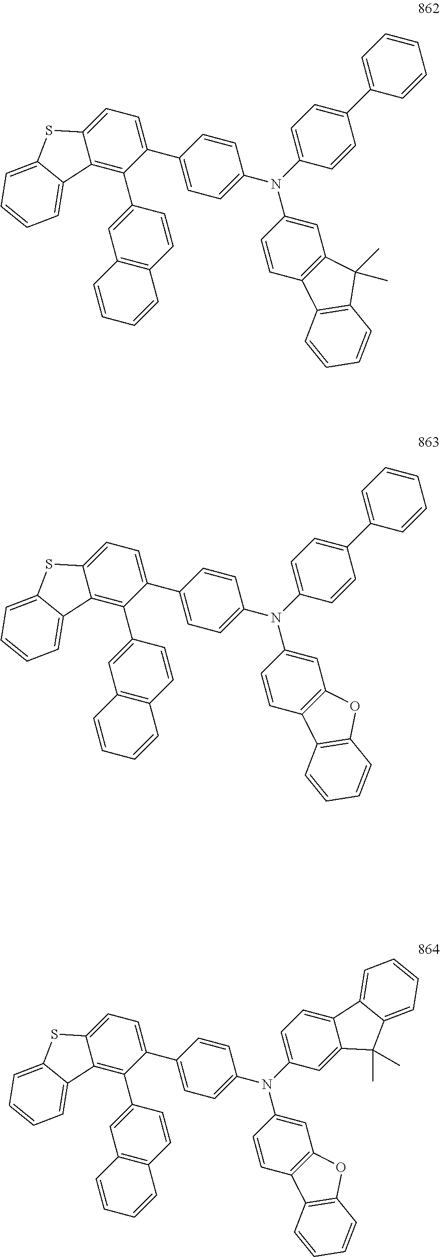 [SMILES: CC1(C)c2ccccc2-c2ccc(N(c3ccc(-c4ccccc4)cc3)c3ccc(-c4ccc5sc6ccccc6c5c4-c4ccc5ccccc5c4)cc3)cc21 ; c1ccc(-c2ccc(N(c3ccc(-c4ccc5sc6ccccc6c5c4-c4ccc5ccccc5c4)cc3)c3ccc4c(c3)oc3ccccc34)cc2)cc1 ; CC1(C)c2ccccc2-c2ccc(N(c3ccc(-c4ccc5sc6ccccc6c5c4-c4ccc5ccccc5c4)cc3)c3ccc4c(c3)oc3ccccc34)cc21]

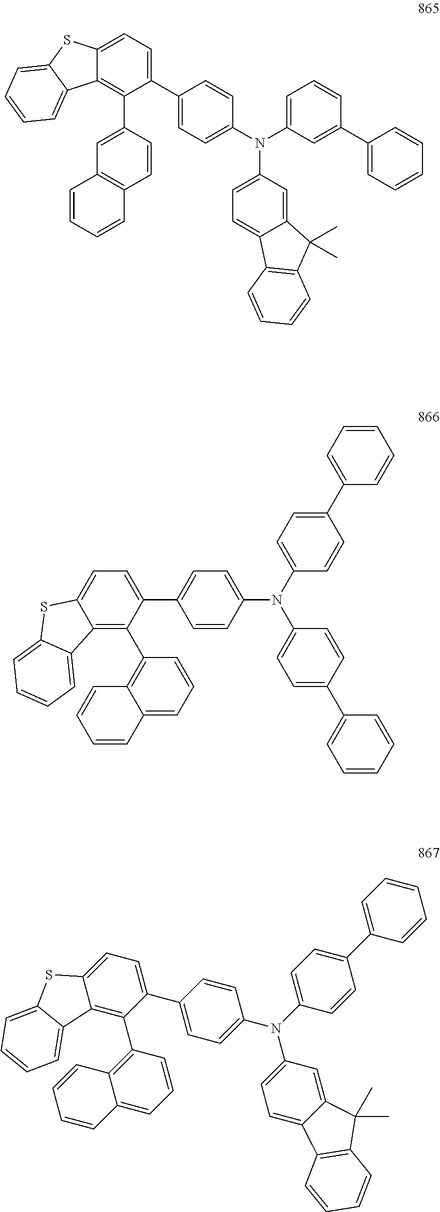 [SMILES: CC1(C)c2ccccc2-c2ccc(N(c3ccc(-c4ccc5sc6ccccc6c5c4-c4ccc5ccccc5c4)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4cccc5ccccc45)cc3)cc2)cc1 ; CC1(C)c2ccccc2-c2ccc(N(c3ccc(-c4ccccc4)cc3)c3ccc(-c4ccc5sc6ccccc6c5c4-c4cccc5ccccc45)cc3)cc21]

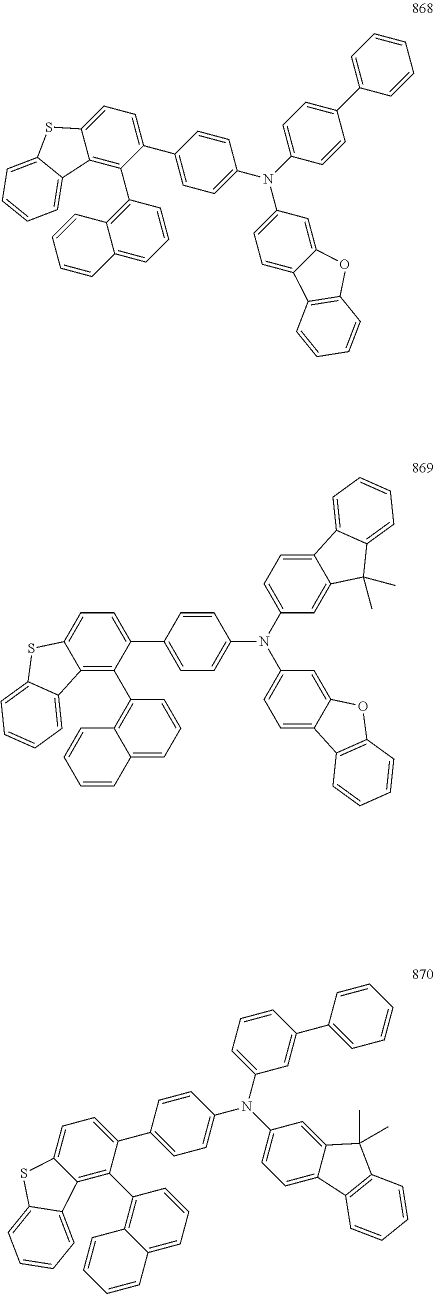 [SMILES: c1ccc(-c2ccc(N(c3ccc(-c4ccc5sc6ccccc6c5c4-c4cccc5ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4cccc5ccccc45)cc3)c3ccc4c(c3)oc3ccccc34)cc21 ; CC1(C)c2ccccc2-c2ccc(N(c3ccc(-c4ccc5sc6ccccc6c5c4-c4cccc5ccccc45)cc3)c3cccc(-c4ccccc4)c3)cc21]

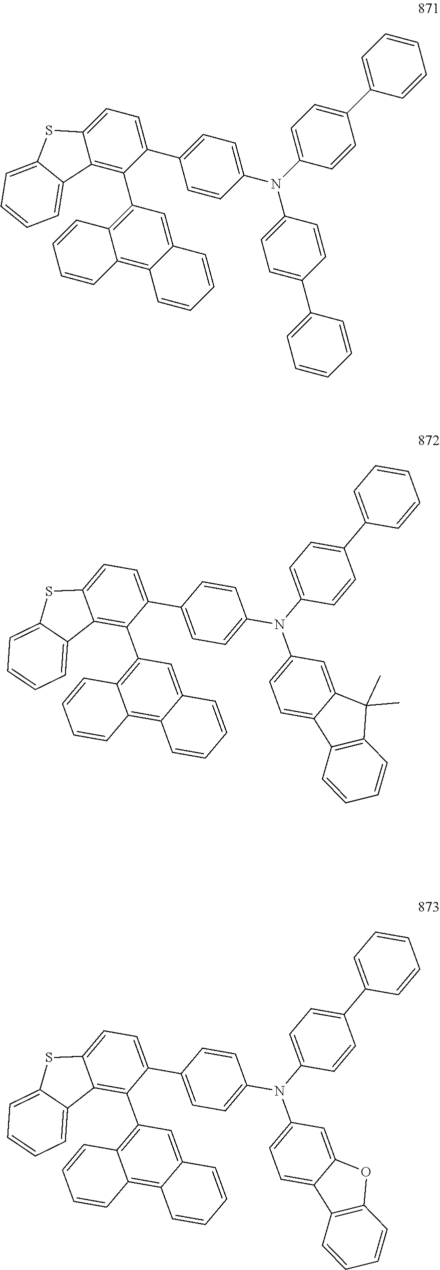 [SMILES: c1ccc(-c2ccc(N(c3ccc(-c4ccccc4)cc3)c3ccc(-c4ccc5sc6ccccc6c5c4-c4cc5ccccc5c5ccccc45)cc3)cc2)cc1 ; CC1(C)c2ccccc2-c2ccc(N(c3ccc(-c4ccccc4)cc3)c3ccc(-c4ccc5sc6ccccc6c5c4-c4cc5ccccc5c5ccccc45)cc3)cc21 ; c1ccc(-c2ccc(N(c3ccc(-c4ccc5sc6ccccc6c5c4-c4cc5ccccc5c5ccccc45)cc3)c3ccc4c(c3)oc3ccccc34)cc2)cc1]

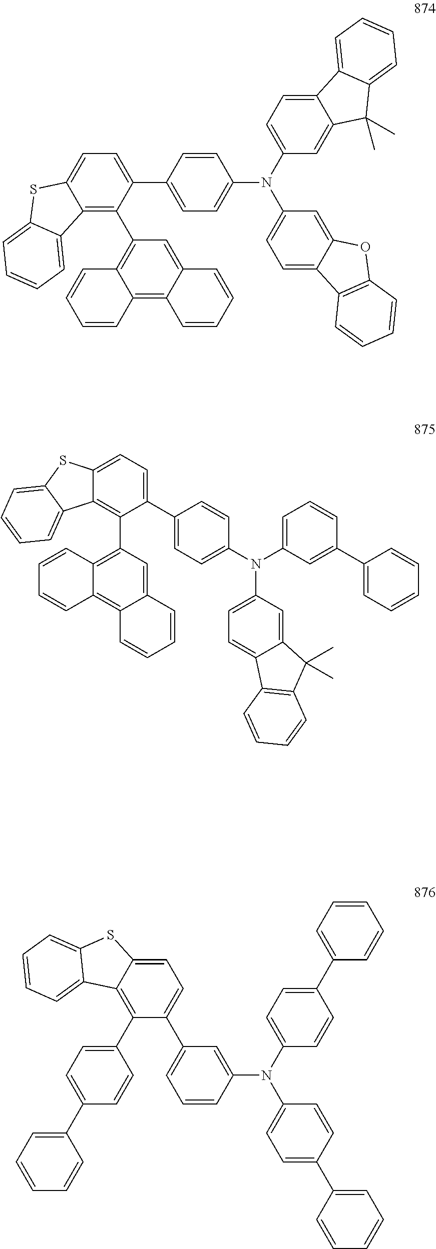 [SMILES: CC1(C)c2ccccc2-c2ccc(N(c3ccc(-c4ccc5sc6ccccc6c5c4-c4cc5ccccc5c5ccccc45)cc3)c3ccc4c(c3)oc3ccccc34)cc21 ; CC1(C)c2ccccc2-c2ccc(N(c3ccc(-c4ccc5sc6ccccc6c5c4-c4cc5ccccc5c5ccccc45)cc3)c3cccc(-c4ccccc4)c3)cc21 ; c1ccc(-c2ccc(-c3c(-c4cccc(N(c5ccc(-c6ccccc6)cc5)c5ccc(-c6ccccc6)cc5)c4)ccc4sc5ccccc5c34)cc2)cc1]

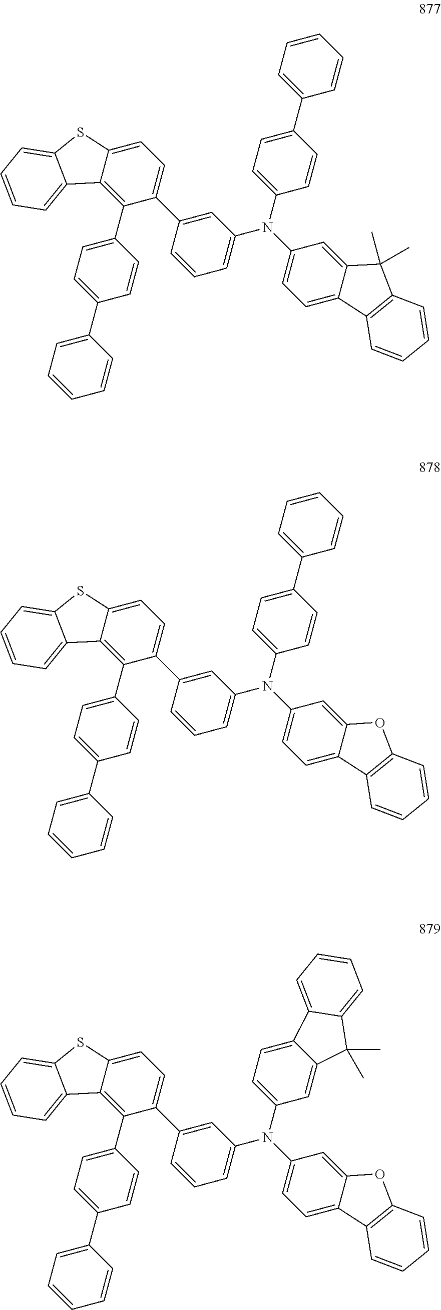 [SMILES: CC1(C)c2ccccc2-c2ccc(N(c3ccc(-c4ccccc4)cc3)c3cccc(-c4ccc5sc6ccccc6c5c4-c4ccc(-c5ccccc5)cc4)c3)cc21 ; c1ccc(-c2ccc(-c3c(-c4cccc(N(c5ccc(-c6ccccc6)cc5)c5ccc6c(c5)oc5ccccc56)c4)ccc4sc5ccccc5c34)cc2)cc1 ; CC1(C)c2ccccc2-c2ccc(N(c3cccc(-c4ccc5sc6ccccc6c5c4-c4ccc(-c5ccccc5)cc4)c3)c3ccc4c(c3)oc3ccccc34)cc21]

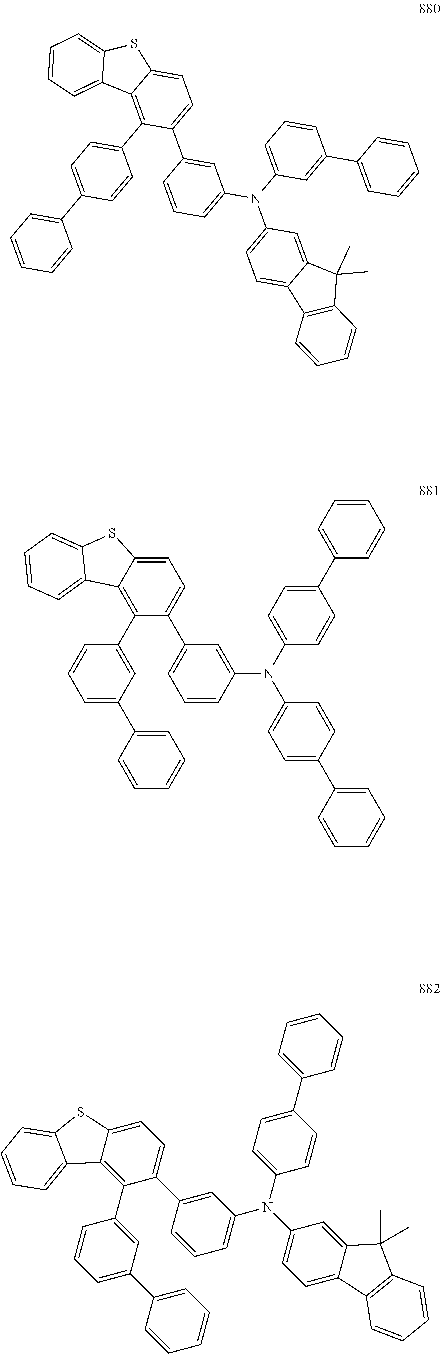 [SMILES: CC1(C)c2ccccc2-c2ccc(N(c3cccc(-c4ccccc4)c3)c3cccc(-c4ccc5sc6ccccc6c5c4-c4ccc(-c5ccccc5)cc4)c3)cc21 ; c1ccc(-c2ccc(N(c3ccc(-c4ccccc4)cc3)c3cccc(-c4ccc5sc6ccccc6c5c4-c4cccc(-c5ccccc5)c4)c3)cc2)cc1 ; CC1(C)c2ccccc2-c2ccc(N(c3ccc(-c4ccccc4)cc3)c3cccc(-c4ccc5sc6ccccc6c5c4-c4cccc(-c5ccccc5)c4)c3)cc21]

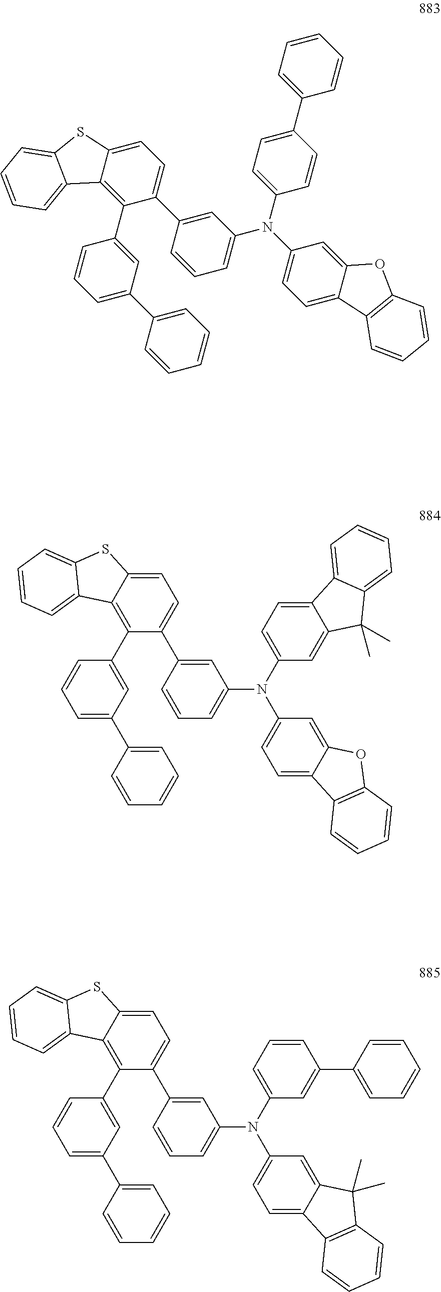 [SMILES: c1ccc(-c2ccc(N(c3cccc(-c4ccc5sc6ccccc6c5c4-c4cccc(-c5ccccc5)c4)c3)c3ccc4c(c3)oc3ccccc34)cc2)cc1 ; CC1(C)c2ccccc2-c2ccc(N(c3cccc(-c4ccc5sc6ccccc6c5c4-c4cccc(-c5ccccc5)c4)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4cccc(-c5ccccc5)c4)c3)cc21]

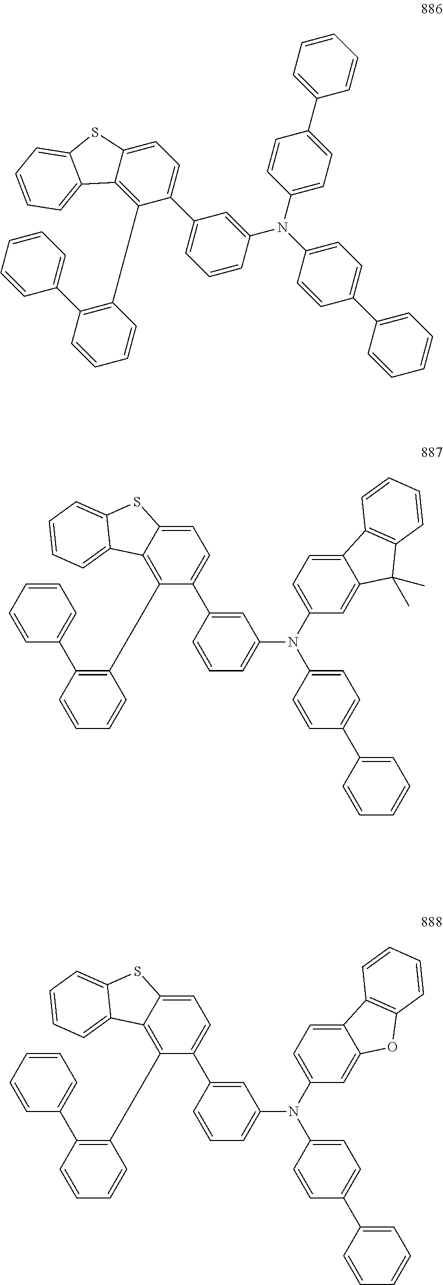 [SMILES: c1ccc(-c2ccc(N(c3ccc(-c4ccccc4)cc3)c3cccc(-c4ccc5sc6ccccc6c5c4-c4ccccc4-c4ccccc4)c3)cc2)cc1 ; CC1(C)c2ccccc2-c2ccc(N(c3ccc(-c4ccccc4)cc3)c3cccc(-c4ccc5sc6ccccc6c5c4-c4ccccc4-c4ccccc4)c3)cc21 ; c1ccc(-c2ccc(N(c3cccc(-c4ccc5sc6ccccc6c5c4-c4ccccc4-c4ccccc4)c3)c3ccc4c(c3)oc3ccccc34)cc2)cc1]

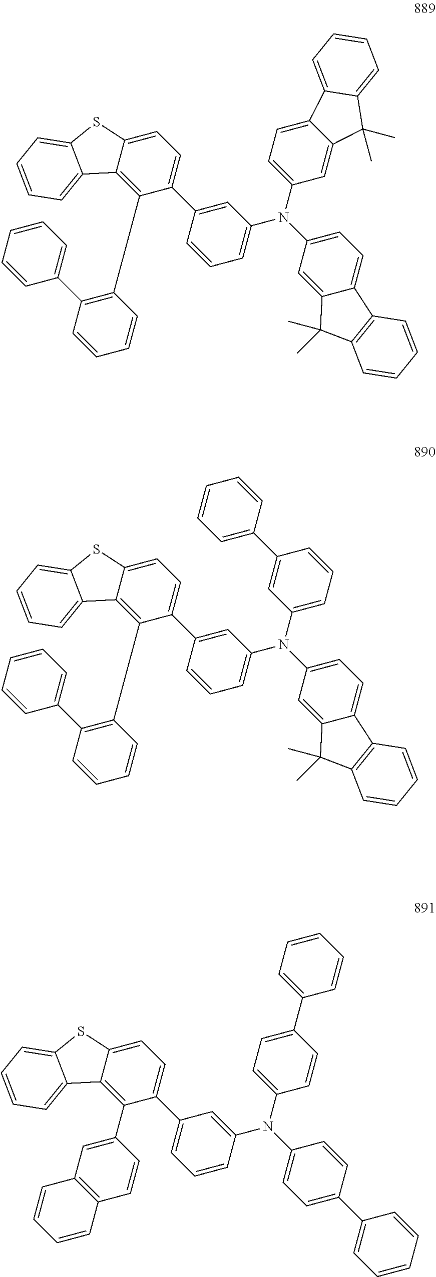 [SMILES: CC1(C)c2ccccc2-c2ccc(N(c3cccc(-c4ccc5sc6ccccc6c5c4-c4ccccc4-c4ccccc4)c3)c3ccc4c(c3)C(C)(C)c3ccccc3-4)cc21 ; CC1(C)c2ccccc2-c2ccc(N(c3cccc(-c4ccccc4)c3)c3cccc(-c4ccc5sc6ccccc6c5c4-c4ccccc4-c4ccccc4)c3)cc21 ; c1ccc(-c2ccc(N(c3ccc(-c4ccccc4)cc3)c3cccc(-c4ccc5sc6ccccc6c5c4-c4ccc5ccccc5c4)c3)cc2)cc1]

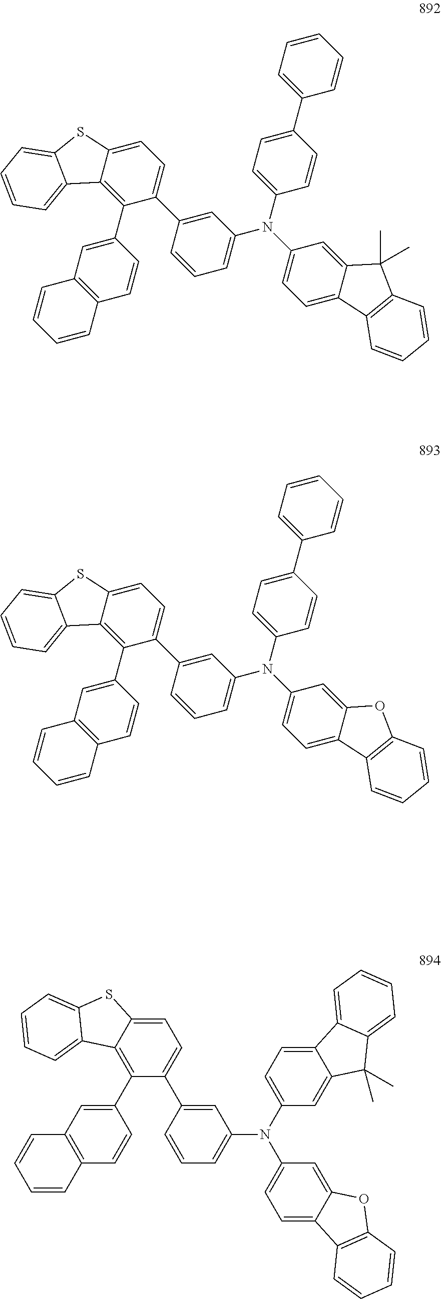 [SMILES: CC1(C)c2ccccc2-c2ccc(N(c3ccc(-c4ccccc4)cc3)c3cccc(-c4ccc5sc6ccccc6c5c4-c4ccc5ccccc5c4)c3)cc21 ; c1ccc(-c2ccc(N(c3cccc(-c4ccc5sc6ccccc6c5c4-c4ccc5ccccc5c4)c3)c3ccc4c(c3)oc3ccccc34)cc2)cc1 ; CC1(C)c2ccccc2-c2ccc(N(c3cccc(-c4ccc5sc6ccccc6c5c4-c4ccc5ccccc5c4)c3)c3ccc4c(c3)oc3ccccc34)cc21]

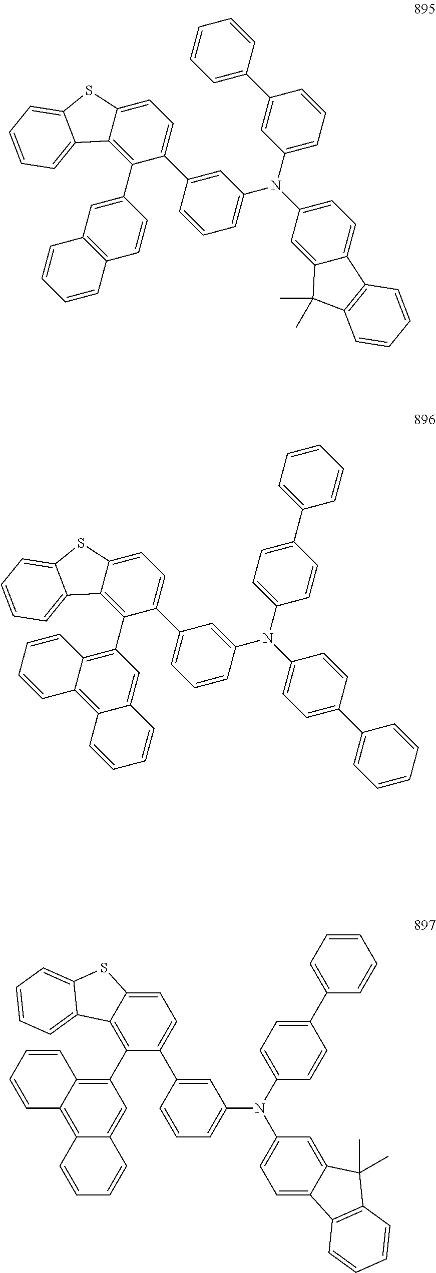 [SMILES: CC1(C)c2ccccc2-c2ccc(N(c3cccc(-c4ccccc4)c3)c3cccc(-c4ccc5sc6ccccc6c5c4-c4ccc5ccccc5c4)c3)cc21 ; c1ccc(-c2ccc(N(c3ccc(-c4ccccc4)cc3)c3cccc(-c4ccc5sc6ccccc6c5c4-c4cc5ccccc5c5ccccc45)c3)cc2)cc1 ; CC1(C)c2ccccc2-c2ccc(N(c3ccc(-c4ccccc4)cc3)c3cccc(-c4ccc5sc6ccccc6c5c4-c4cc5ccccc5c5ccccc45)c3)cc21]

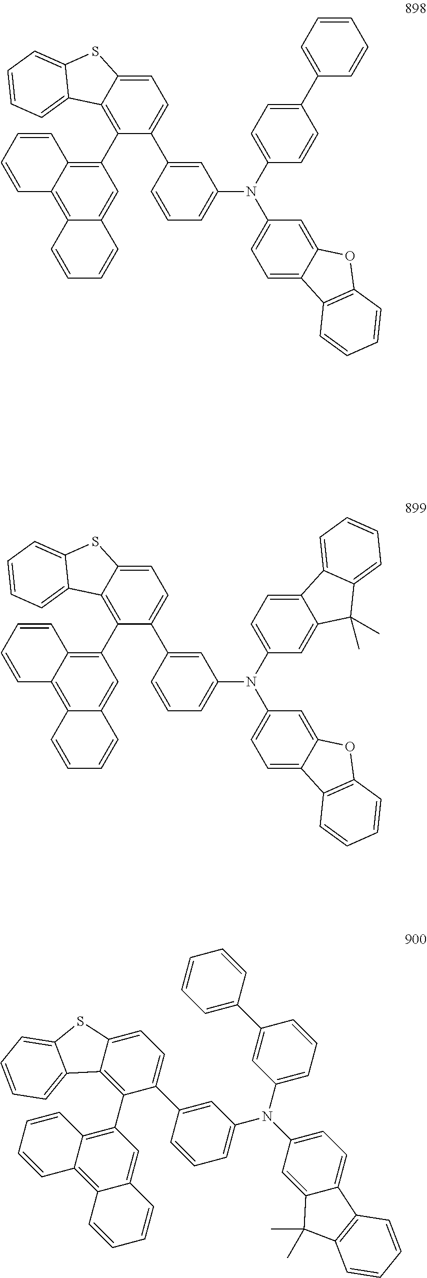 [SMILES: c1ccc(-c2ccc(N(c3cccc(-c4ccc5sc6ccccc6c5c4-c4cc5ccccc5c5ccccc45)c3)c3ccc4c(c3)oc3ccccc34)cc2)cc1 ; CC1(C)c2ccccc2-c2ccc(N(c3cccc(-c4ccc5sc6ccccc6c5c4-c4cc5ccccc5c5ccccc45)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4cc5ccccc5c5ccccc45)c3)cc21]

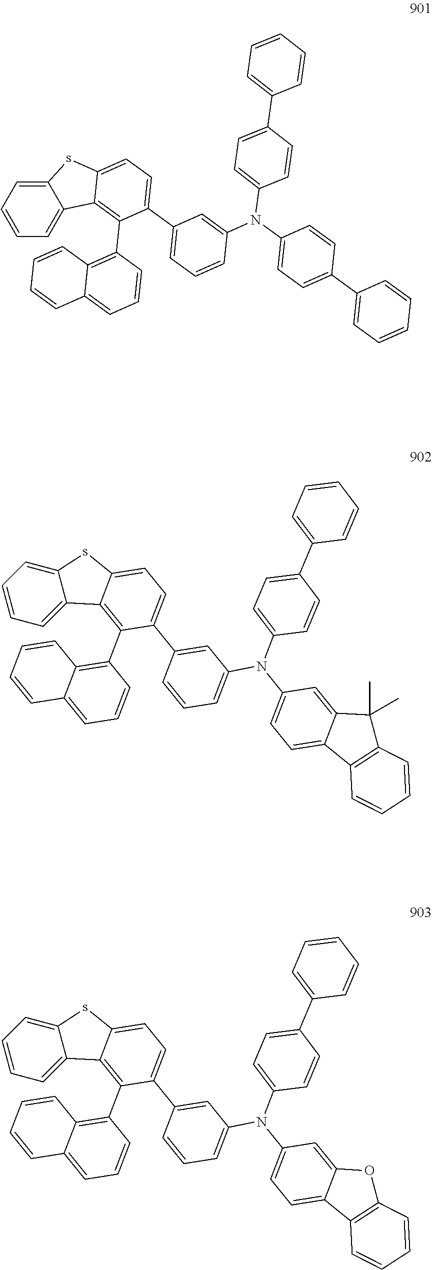

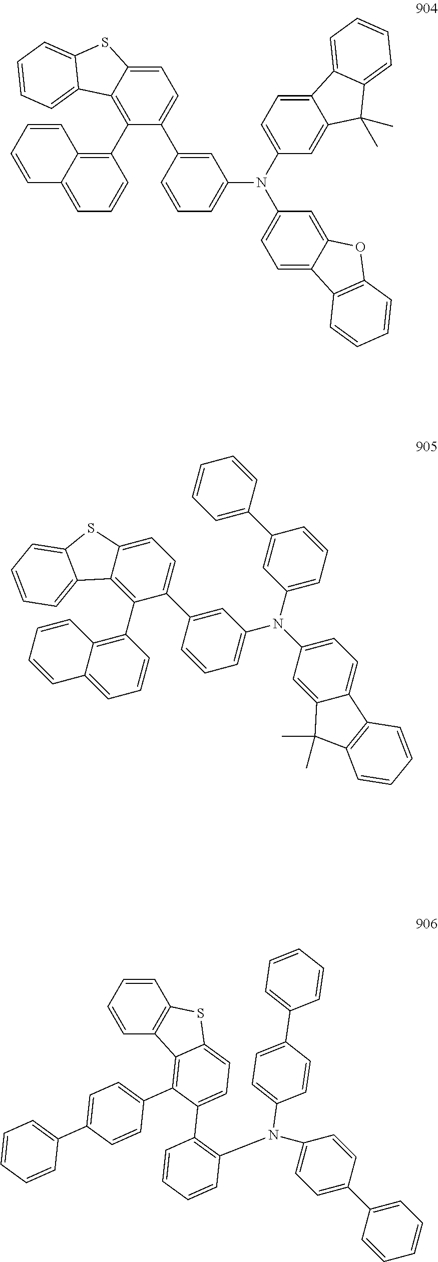 [SMILES: CC1(C)c2ccccc2-c2ccc(N(c3cccc(-c4ccc5sc6ccccc6c5c4-c4cccc5ccccc45)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4cccc5ccccc45)c3)cc21 ; c1ccc(-c2ccc(-c3c(-c4ccccc4N(c4ccc(-c5ccccc5)cc4)c4ccc(-c5ccccc5)cc4)ccc4sc5ccccc5c34)cc2)cc1]

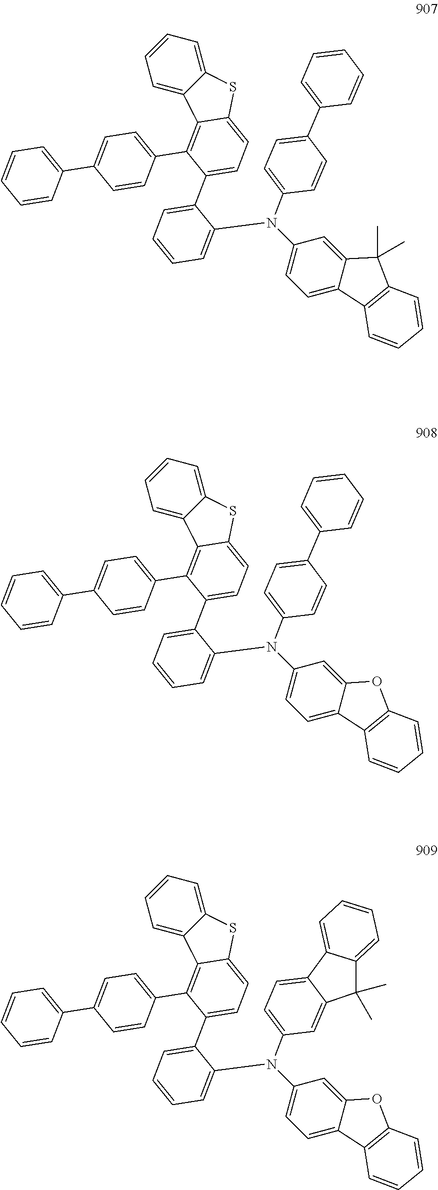 [SMILES: CC1(C)c2ccccc2-c2ccc(N(c3ccc(-c4ccccc4)cc3)c3ccccc3-c3ccc4sc5ccccc5c4c3-c3ccc(-c4ccccc4)cc3)cc21 ; c1ccc(-c2ccc(-c3c(-c4ccccc4N(c4ccc(-c5ccccc5)cc4)c4ccc5c(c4)oc4ccccc45)ccc4sc5ccccc5c34)cc2)cc1 ; CC1(C)c2ccccc2-c2ccc(N(c3ccc4c(c3)oc3ccccc34)c3ccccc3-c3ccc4sc5ccccc5c4c3-c3ccc(-c4ccccc4)cc3)cc21]

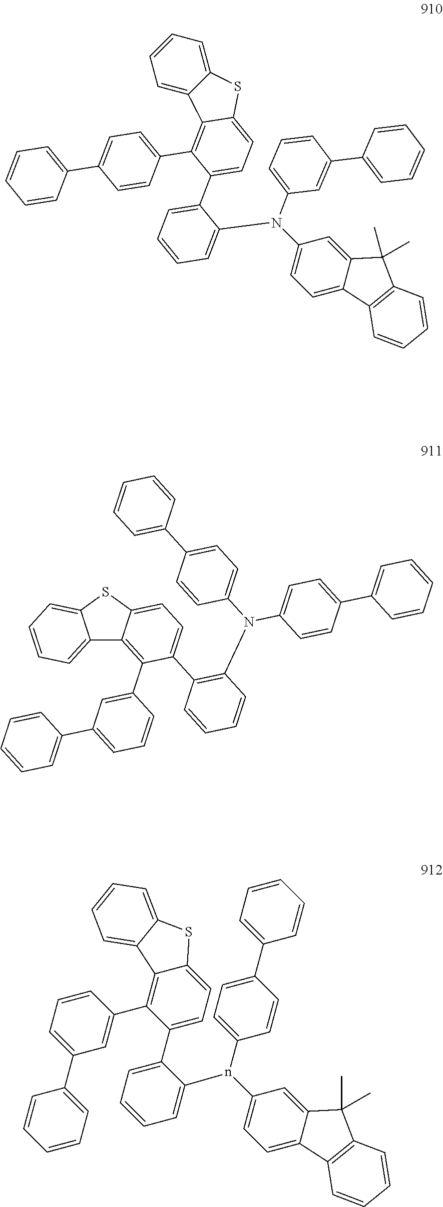

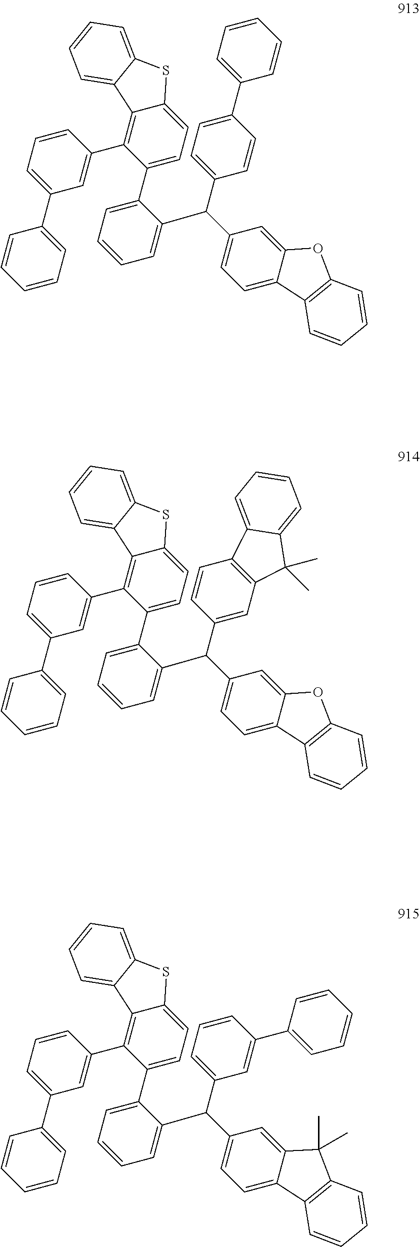 [SMILES: c1ccc(-c2ccc(C(c3ccc4c(c3)oc3ccccc34)c3ccccc3-c3ccc4sc5ccccc5c4c3-c3cccc(-c4ccccc4)c3)cc2)cc1 ; CC1(C)c2ccccc2-c2ccc(C(c3ccc4c(c3)oc3ccccc34)c3ccccc3-c3ccc4sc5ccccc5c4c3-c3cccc(-c4ccccc4)c3)cc21 ; CC1(C)c2ccccc2-c2ccc(C(c3cccc(-c4ccccc4)c3)c3ccccc3-c3ccc4sc5ccccc5c4c3-c3cccc(-c4ccccc4)c3)cc21]

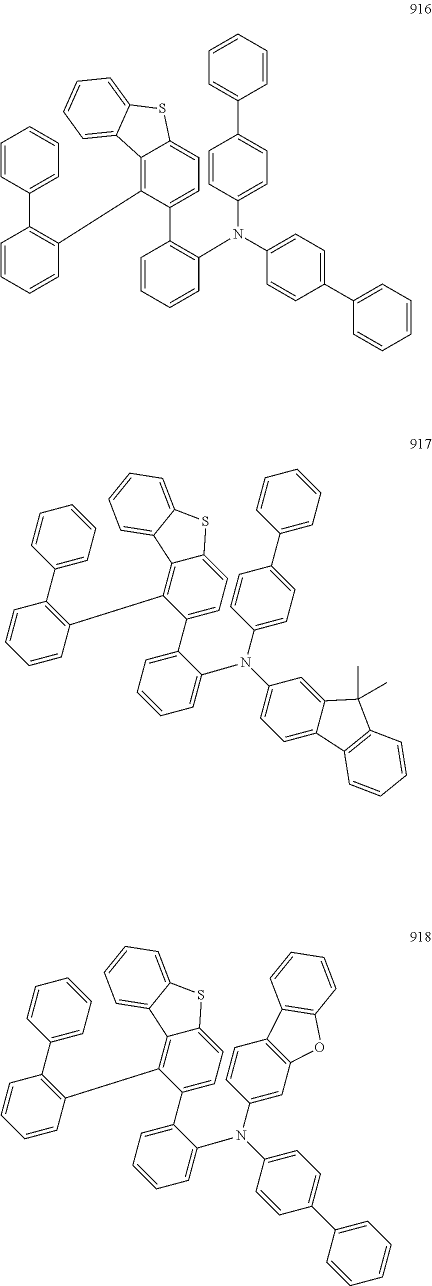 [SMILES: c1ccc(-c2ccc(N(c3ccc(-c4ccccc4)cc3)c3ccccc3-c3ccc4sc5ccccc5c4c3-c3ccccc3-c3ccccc3)cc2)cc1 ; CC1(C)c2ccccc2-c2ccc(N(c3ccc(-c4ccccc4)cc3)c3ccccc3-c3ccc4sc5ccccc5c4c3-c3ccccc3-c3ccccc3)cc21 ; c1ccc(-c2ccc(N(c3ccc4c(c3)oc3ccccc34)c3ccccc3-c3ccc4sc5ccccc5c4c3-c3ccccc3-c3ccccc3)cc2)cc1]

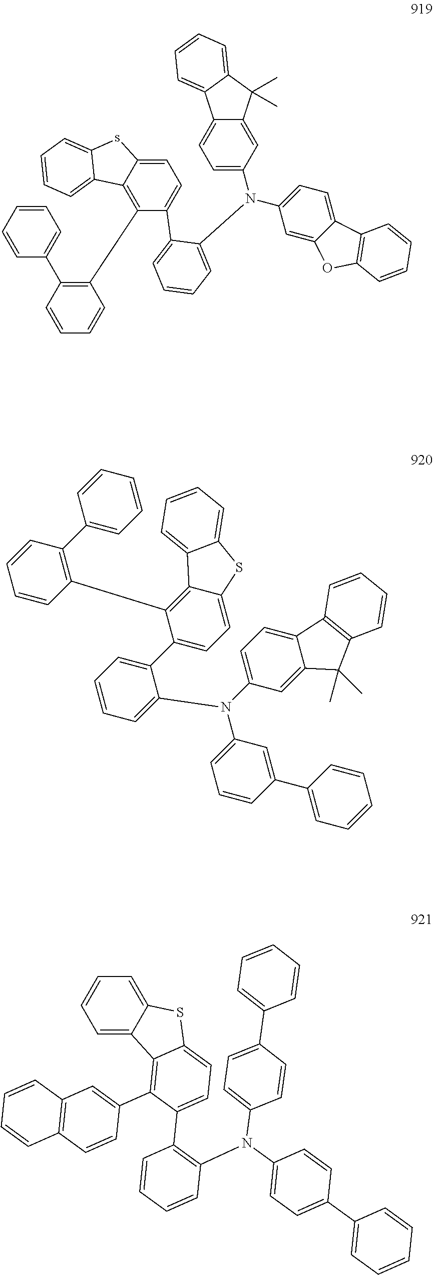

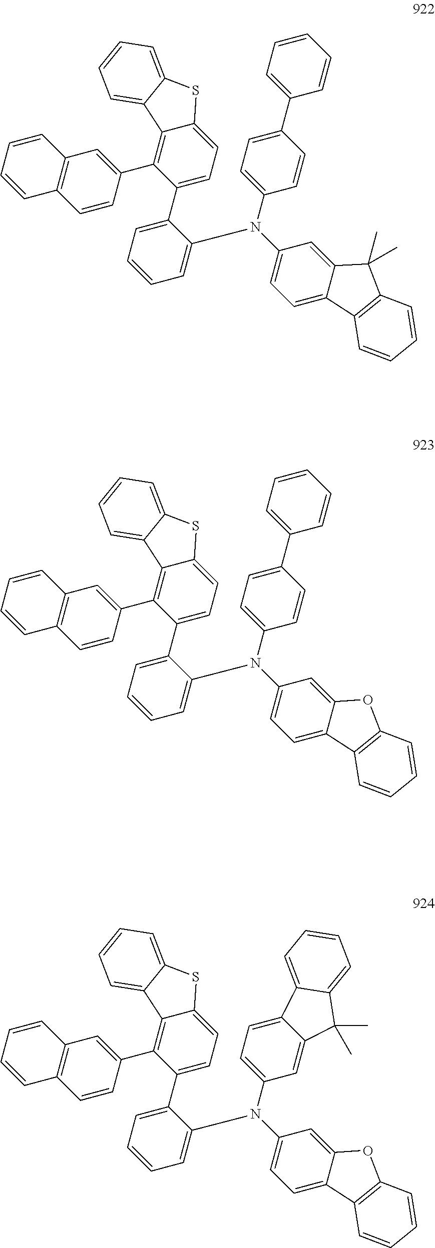 [SMILES: CC1(C)c2ccccc2-c2ccc(N(c3ccc(-c4ccccc4)cc3)c3ccccc3-c3ccc4sc5ccccc5c4c3-c3ccc4ccccc4c3)cc21 ; c1ccc(-c2ccc(N(c3ccc4c(c3)oc3ccccc34)c3ccccc3-c3ccc4sc5ccccc5c4c3-c3ccc4ccccc4c3)cc2)cc1 ; CC1(C)c2ccccc2-c2ccc(N(c3ccc4c(c3)oc3ccccc34)c3ccccc3-c3ccc4sc5ccccc5c4c3-c3ccc4ccccc4c3)cc21]

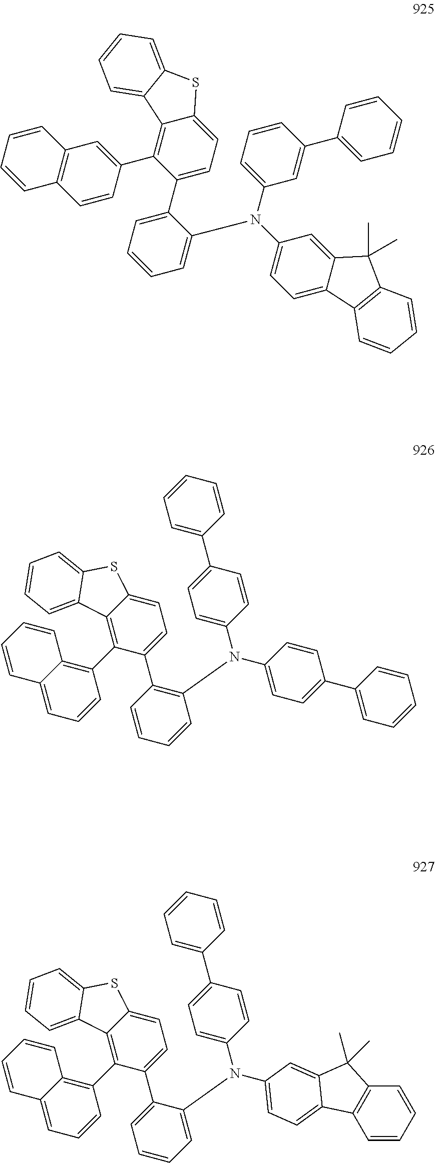 [SMILES: CC1(C)c2ccccc2-c2ccc(N(c3cccc(-c4ccccc4)c3)c3ccccc3-c3ccc4sc5ccccc5c4c3-c3ccc4ccccc4c3)cc21 ; c1ccc(-c2ccc(N(c3ccc(-c4ccccc4)cc3)c3ccccc3-c3ccc4sc5ccccc5c4c3-c3cccc4ccccc34)cc2)cc1 ; CC1(C)c2ccccc2-c2ccc(N(c3ccc(-c4ccccc4)cc3)c3ccccc3-c3ccc4sc5ccccc5c4c3-c3cccc4ccccc34)cc21]

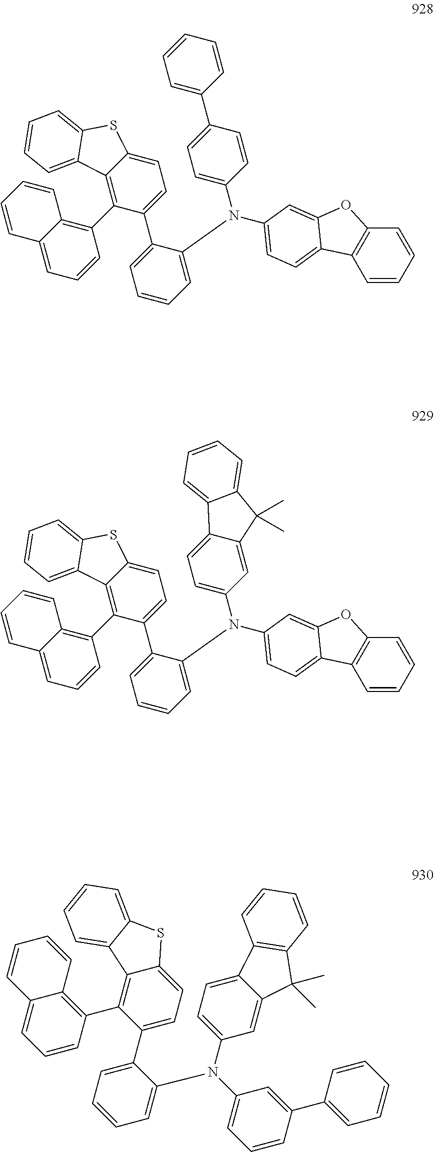 [SMILES: c1ccc(-c2ccc(N(c3ccc4c(c3)oc3ccccc34)c3ccccc3-c3ccc4sc5ccccc5c4c3-c3cccc4ccccc34)cc2)cc1 ; CC1(C)c2ccccc2-c2ccc(N(c3ccc4c(c3)oc3ccccc34)c3ccccc3-c3ccc4sc5ccccc5c4c3-c3cccc4ccccc34)cc21 ; CC1(C)c2ccccc2-c2ccc(N(c3cccc(-c4ccccc4)c3)c3ccccc3-c3ccc4sc5ccccc5c4c3-c3cccc4ccccc34)cc21]

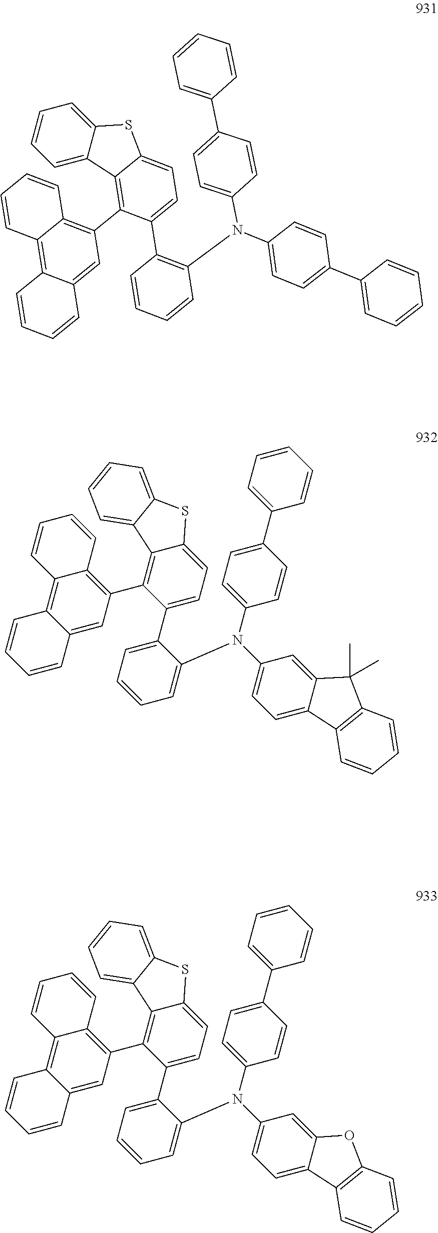 [SMILES: c1ccc(-c2ccc(N(c3ccc(-c4ccccc4)cc3)c3ccccc3-c3ccc4sc5ccccc5c4c3-c3cc4ccccc4c4ccccc34)cc2)cc1 ; CC1(C)c2ccccc2-c2ccc(N(c3ccc(-c4ccccc4)cc3)c3ccccc3-c3ccc4sc5ccccc5c4c3-c3cc4ccccc4c4ccccc34)cc21 ; c1ccc(-c2ccc(N(c3ccc4c(c3)oc3ccccc34)c3ccccc3-c3ccc4sc5ccccc5c4c3-c3cc4ccccc4c4ccccc34)cc2)cc1]

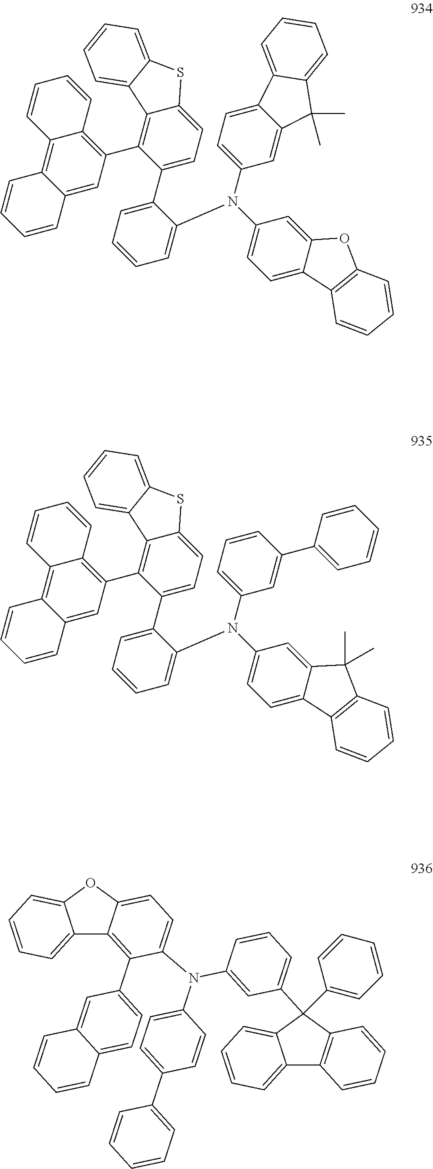 [SMILES: CC1(C)c2ccccc2-c2ccc(N(c3ccc4c(c3)oc3ccccc34)c3ccccc3-c3ccc4sc5ccccc5c4c3-c3cc4ccccc4c4ccccc34)cc21 ; CC1(C)c2ccccc2-c2ccc(N(c3cccc(-c4ccccc4)c3)c3ccccc3-c3ccc4sc5ccccc5c4c3-c3cc4ccccc4c4ccccc34)cc21 ; c1ccc(-c2ccc(N(c3cccc(C4(c5ccccc5)c5ccccc5-c5ccccc54)c3)c3ccc4oc5ccccc5c4c3-c3ccc4ccccc4c3)cc2)cc1]

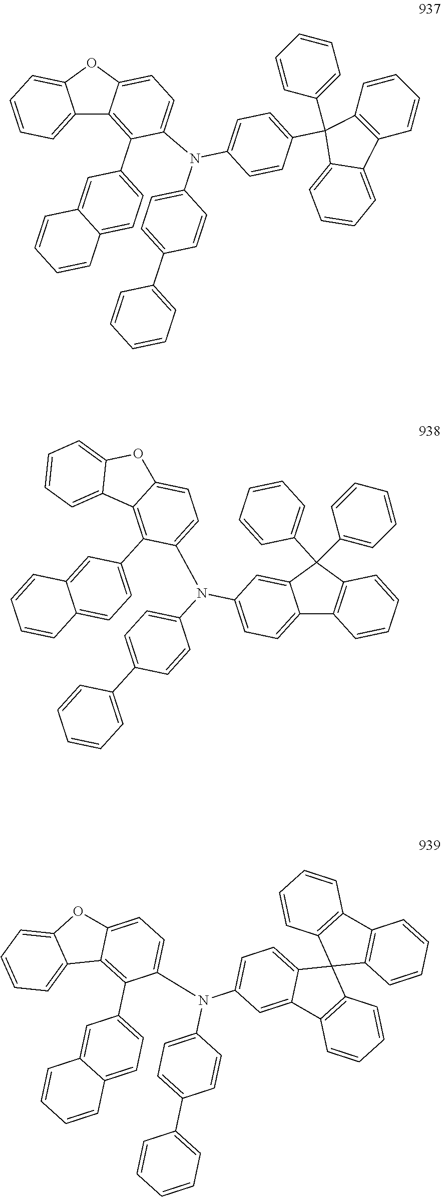 [SMILES: c1ccc(-c2ccc(N(c3ccc(C4(c5ccccc5)c5ccccc5-c5ccccc54)cc3)c3ccc4oc5ccccc5c4c3-c3ccc4ccccc4c3)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4ccccc4c3)cc2)cc1 ; c1ccc(-c2ccc(N(c3ccc4c(c3)-c3ccccc3C43c4ccccc4-c4ccccc43)c3ccc4oc5ccccc5c4c3-c3ccc4ccccc4c3)cc2)cc1]

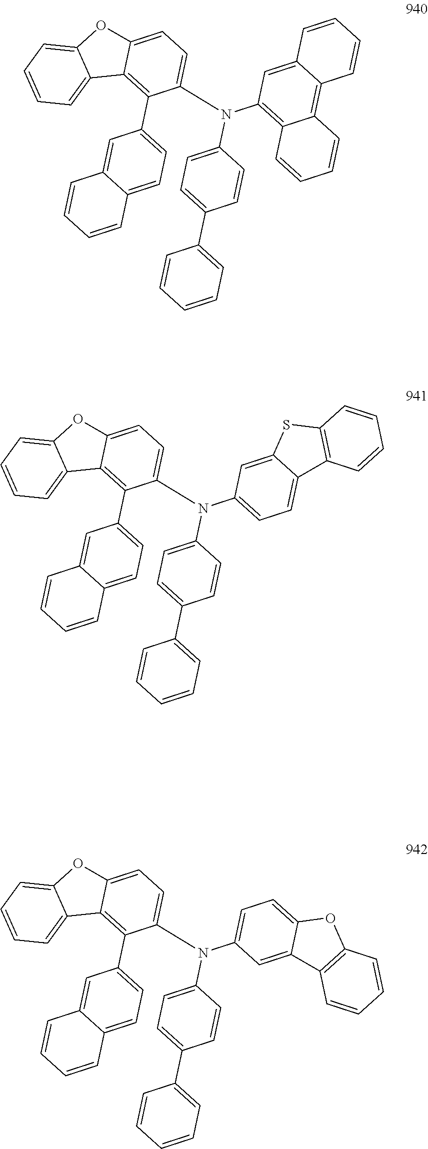 [SMILES: c1ccc(-c2ccc(N(c3ccc4oc5ccccc5c4c3-c3ccc4ccccc4c3)c3cc4ccccc4c4ccccc34)cc2)cc1 ; c1ccc(-c2ccc(N(c3ccc4c(c3)sc3ccccc34)c3ccc4oc5ccccc5c4c3-c3ccc4ccccc4c3)cc2)cc1 ; c1ccc(-c2ccc(N(c3ccc4oc5ccccc5c4c3)c3ccc4oc5ccccc5c4c3-c3ccc4ccccc4c3)cc2)cc1]

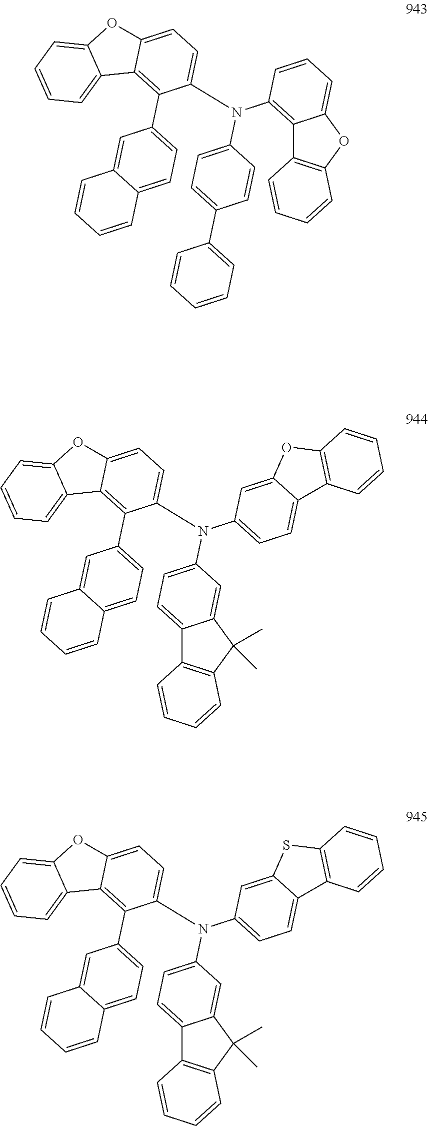 [SMILES: c1ccc(-c2ccc(N(c3ccc4oc5ccccc5c4c3-c3ccc4ccccc4c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4ccccc4c3)cc21 ; CC1(C)c2ccccc2-c2ccc(N(c3ccc4c(c3)sc3ccccc34)c3ccc4oc5ccccc5c4c3-c3ccc4ccccc4c3)cc21]

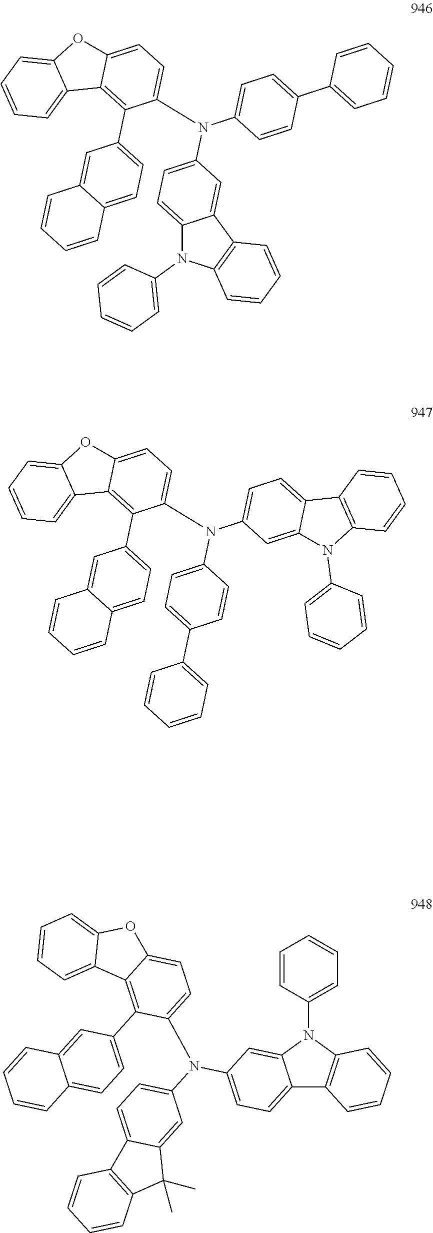 [SMILES: c1ccc(-c2ccc(N(c3ccc4c(c3)c3ccccc3n4-c3ccccc3)c3ccc4oc5ccccc5c4c3-c3ccc4ccccc4c3)cc2)cc1 ; c1ccc(-c2ccc(N(c3ccc4c5ccccc5n(-c5ccccc5)c4c3)c3ccc4oc5ccccc5c4c3-c3ccc4ccccc4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4ccccc4c3)cc21]

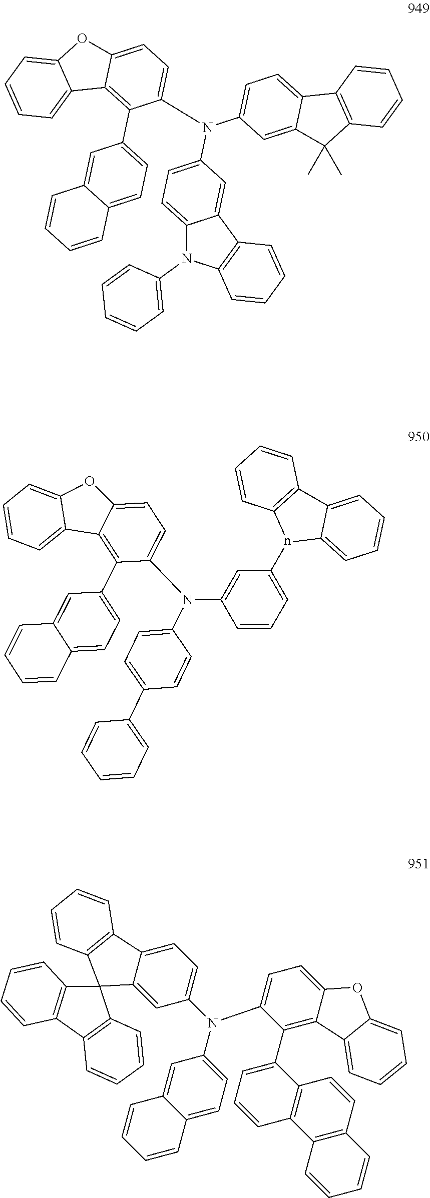

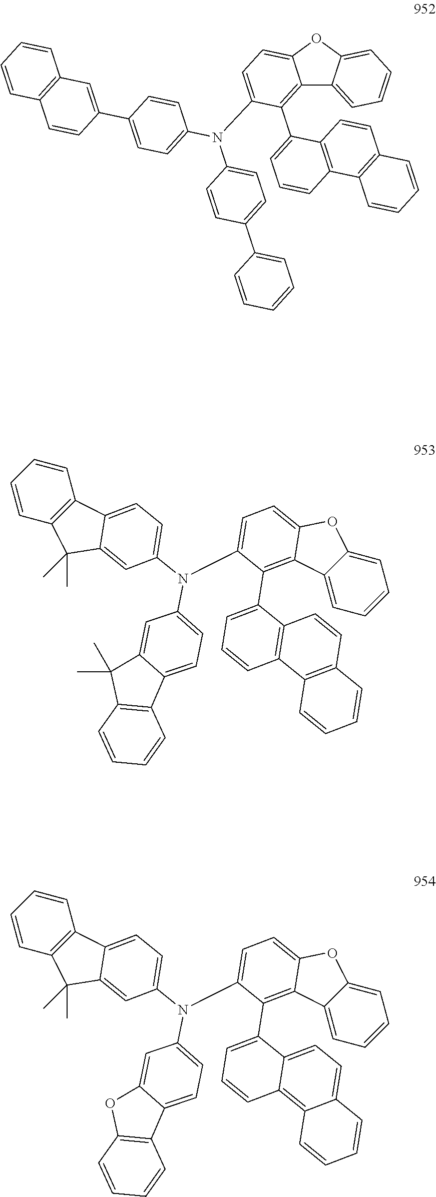 [SMILES: c1ccc(-c2ccc(N(c3ccc(-c4ccc5ccccc5c4)cc3)c3ccc4oc5ccccc5c4c3-c3cccc4c3ccc3ccccc34)cc2)cc1 ; CC1(C)c2ccccc2-c2ccc(N(c3ccc4c(c3)C(C)(C)c3ccccc3-4)c3ccc4oc5ccccc5c4c3-c3cccc4c3ccc3ccccc34)cc21 ; CC1(C)c2ccccc2-c2ccc(N(c3ccc4c(c3)oc3ccccc34)c3ccc4oc5ccccc5c4c3-c3cccc4c3ccc3ccccc34)cc21]

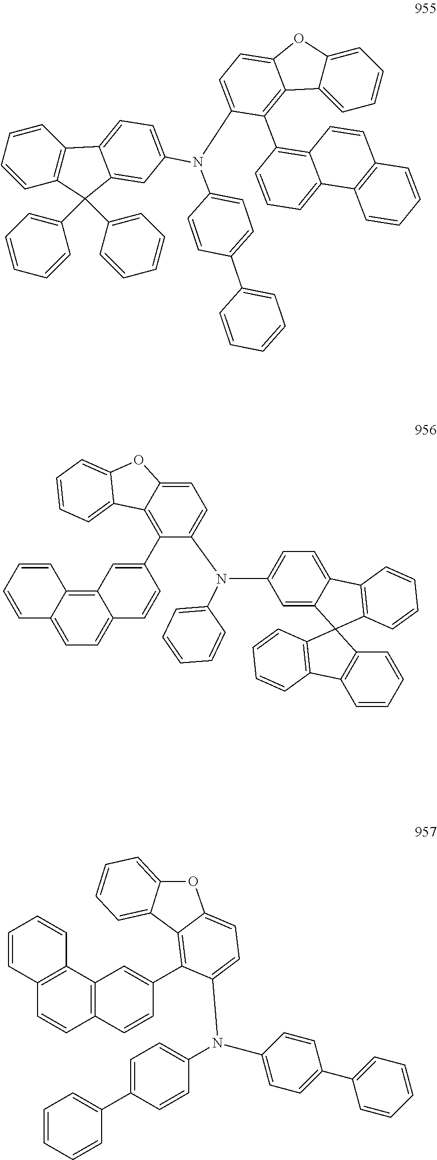 [SMILES: c1ccc(-c2ccc(N(c3ccc4c(c3)C(c3ccccc3)(c3ccccc3)c3ccccc3-4)c3ccc4oc5ccccc5c4c3-c3cccc4c3ccc3ccccc34)cc2)cc1 ; c1ccc(N(c2ccc3c(c2)C2(c4ccccc4-c4ccccc42)c2ccccc2-3)c2ccc3oc4ccccc4c3c2-c2ccc3ccc4ccccc4c3c2)cc1 ; c1ccc(-c2ccc(N(c3ccc(-c4ccccc4)cc3)c3ccc4oc5ccccc5c4c3-c3ccc4ccc5ccccc5c4c3)cc2)cc1]

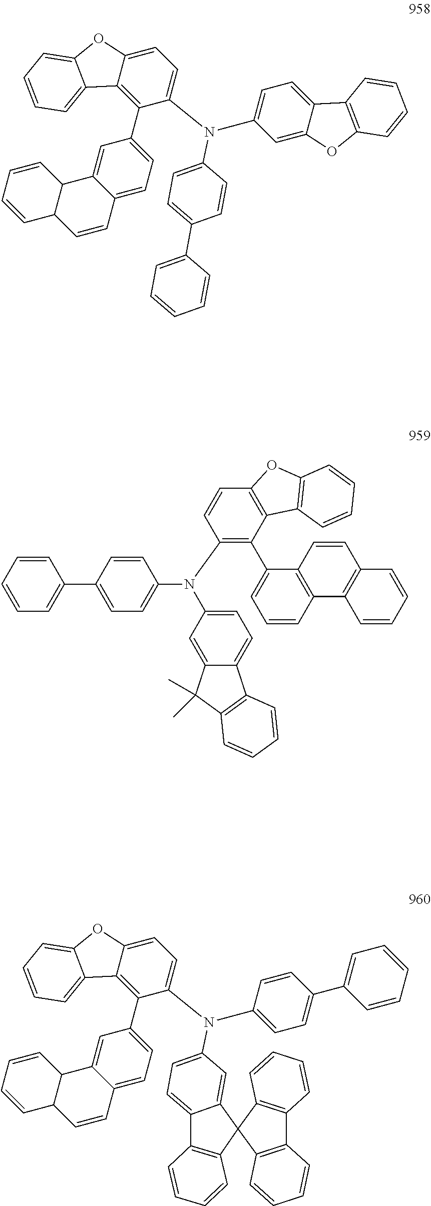 [SMILES: C1=CC2C=Cc3ccc(-c4c(N(c5ccc(-c6ccccc6)cc5)c5ccc6c(c5)oc5ccccc56)ccc5oc6ccccc6c45)cc3C2C=C1 ; CC1(C)c2ccccc2-c2ccc(N(c3ccc(-c4ccccc4)cc3)c3ccc4oc5ccccc5c4c3-c3cccc4c3ccc3ccccc34)cc21 ; C1=CC2C=Cc3ccc(-c4c(N(c5ccc(-c6ccccc6)cc5)c5ccc6c(c5)C5(c7ccccc7-c7ccccc75)c5ccccc5-6)ccc5oc6ccccc6c45)cc3C2C=C1]

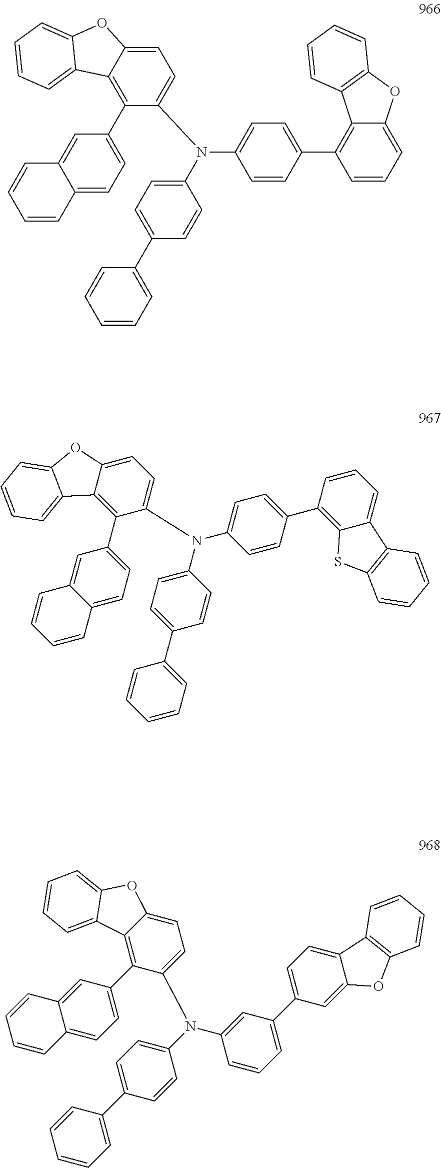 [SMILES: c1ccc(-c2ccc(N(c3ccc(-c4cccc5oc6ccccc6c45)cc3)c3ccc4oc5ccccc5c4c3-c3ccc4ccccc4c3)cc2)cc1 ; c1ccc(-c2ccc(N(c3ccc(-c4cccc5c4sc4ccccc45)cc3)c3ccc4oc5ccccc5c4c3-c3ccc4ccccc4c3)cc2)cc1 ; c1ccc(-c2ccc(N(c3cccc(-c4ccc5c(c4)oc4ccccc45)c3)c3ccc4oc5ccccc5c4c3-c3ccc4ccccc4c3)cc2)cc1]

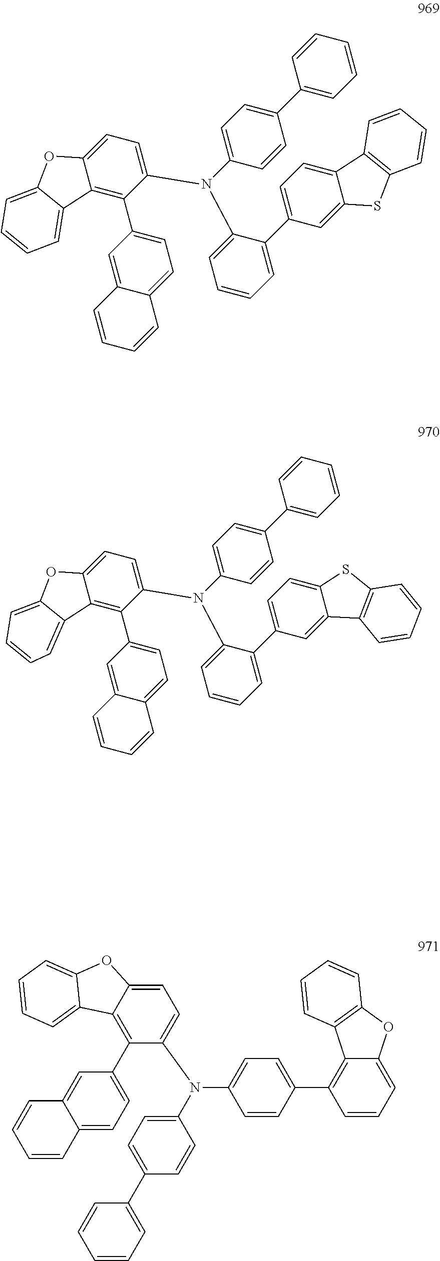 [SMILES: c1ccc(-c2ccc(N(c3ccccc3-c3ccc4c(c3)sc3ccccc34)c3ccc4oc5ccccc5c4c3-c3ccc4ccccc4c3)cc2)cc1 ; c1ccc(-c2ccc(N(c3ccccc3-c3ccc4sc5ccccc5c4c3)c3ccc4oc5ccccc5c4c3-c3ccc4ccccc4c3)cc2)cc1 ; c1ccc(-c2ccc(N(c3ccc(-c4cccc5oc6ccccc6c45)cc3)c3ccc4oc5ccccc5c4c3-c3ccc4ccccc4c3)cc2)cc1]

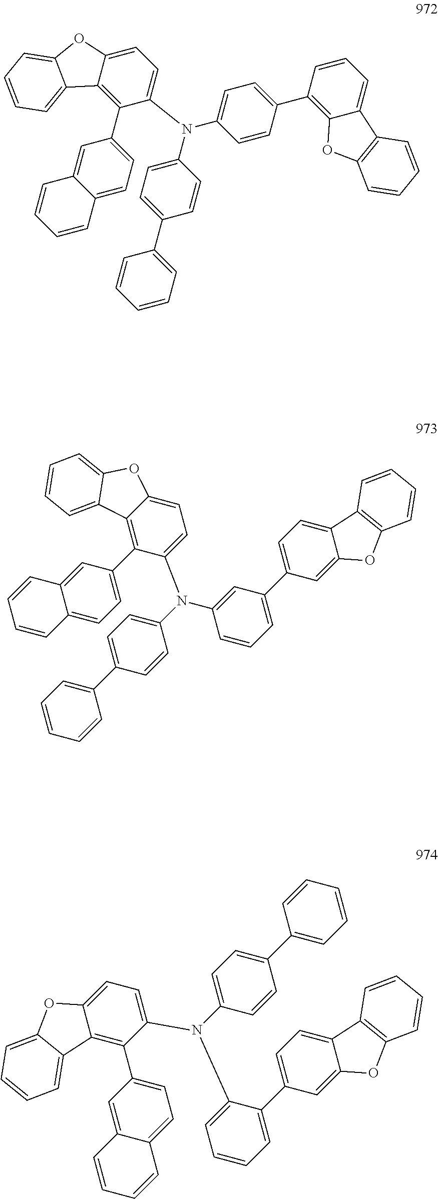 [SMILES: c1ccc(-c2ccc(N(c3ccc(-c4cccc5c4oc4ccccc45)cc3)c3ccc4oc5ccccc5c4c3-c3ccc4ccccc4c3)cc2)cc1 ; c1ccc(-c2ccc(N(c3cccc(-c4ccc5c(c4)oc4ccccc45)c3)c3ccc4oc5ccccc5c4c3-c3ccc4ccccc4c3)cc2)cc1 ; c1ccc(-c2ccc(N(c3ccccc3-c3ccc4c(c3)oc3ccccc34)c3ccc4oc5ccccc5c4c3-c3ccc4ccccc4c3)cc2)cc1]

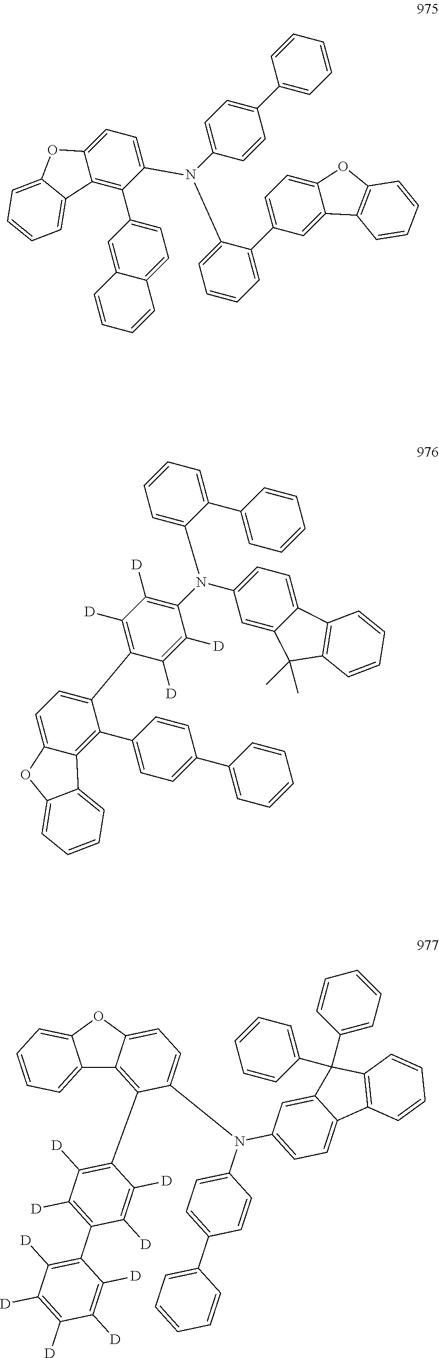 [SMILES: c1ccc(-c2ccc(N(c3ccccc3-c3ccc4oc5ccccc5c4c3)c3ccc4oc5ccccc5c4c3-c3ccc4ccccc4c3)cc2)cc1 ; [2H]c1c([2H])c(N(c2ccc3c(c2)C(C)(C)c2ccccc2-3)c2ccccc2-c2ccccc2)c([2H])c([2H])c1-c1ccc2oc3ccccc3c2c1-c1ccc(-c2ccccc2)cc1 ; [2H]c1c([2H])c([2H])c(-c2c([2H])c([2H])c(-c3c(N(c4ccc(-c5ccccc5)cc4)c4ccc5c(c4)C(c4ccccc4)(c4ccccc4)c4ccccc4-5)ccc4oc5ccccc5c34)c([2H])c2[2H])c([2H])c1[2H]]

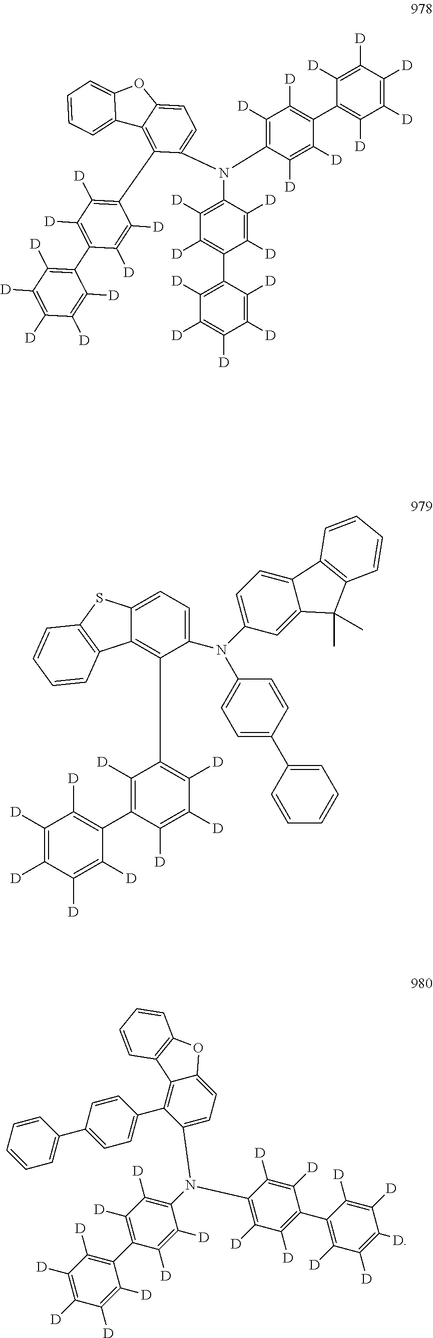

In a second aspect, the present disclosure provides an organic electroluminescent device, including an anode, a cathode, and a functional layer arranged between the anode and the cathode, where the functional layer contains the aromatic amine compound described in the first aspect of the present disclosure.

The aromatic amine compound provided in the present disclosure can be used for forming at least one organic film layer in the functional layer to improve the properties of the organic electroluminescent device, such as luminous efficiency and lifetime.

Optionally, the functional layer includes a hole auxiliary layer, which includes the aromatic amine compound. The hole auxiliary layer may be composed of either the aromatic amine compound provided in the present disclosure or a combination of the aromatic amine compound provided in the present disclosure and other materials.

Optionally, the functional layer further includes a hole transport area, the hole transport area includes a hole transport layer (also known as a first hole transport layer) and a hole auxiliary layer (also known as a second hole transport layer or hole adjustment layer), the hole transport layer is located between the anode and an organic luminescent layer, and the hole adjustment layer is located between the hole transport layer and the organic luminescent layer. In some embodiments, the hole adjustment layer is composed of either the aromatic amine compound provided in the present disclosure or a combination of the aromatic amine compound provided in the present disclosure and other materials.

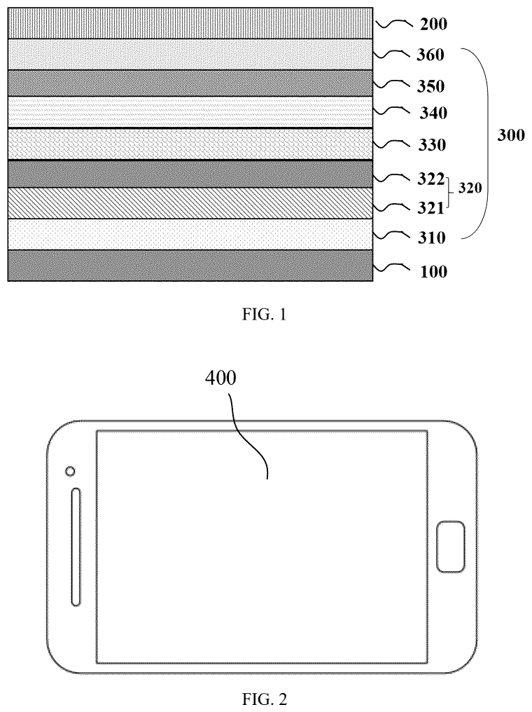

According to a specific embodiment, the organic electroluminescent device, as shown in FIG. 1, includes an anode 100, a hole injection layer 310, a hole transport layer 321, a hole adjustment layer 322, an organic luminescent layer 330, a hole blocking layer 340, an electron transport layer 350, an electron injection layer 360, and a cathode 200 set in sequence.

In the present disclosure, the anode 100 includes an anode material, which is preferably a material with a large work function that facilitates hole injection into the functional layer. Specific examples of the anode material include, but are not limited to, metals such as nickel, platinum, vanadium, chromium, copper, zinc, and gold, or alloys thereof; metal oxides such as zinc oxide, indium oxide, indium tin oxide (ITO), and indium zinc oxide (IZO); combined metals and oxides, such as ZnO:Al or SnO₂:Sb; or conducting polymers, such as poly(3-methylthiophene), poly[3,4-(ethylidene-1,2-dioxy)thiophene] (PEDT), polypyrrole, and polyaniline. A transparent electrode containing indium tin oxide (ITO) is preferably included as the anode.

In the present disclosure, the hole transport layer and the hole adjustment layer may each include one or more hole transport materials, and the hole transport materials may be selected from carbazole polymers, carbazole linked triarylamine compounds, or other types of compounds, and specifically can be selected from the following compounds or any combination thereof:

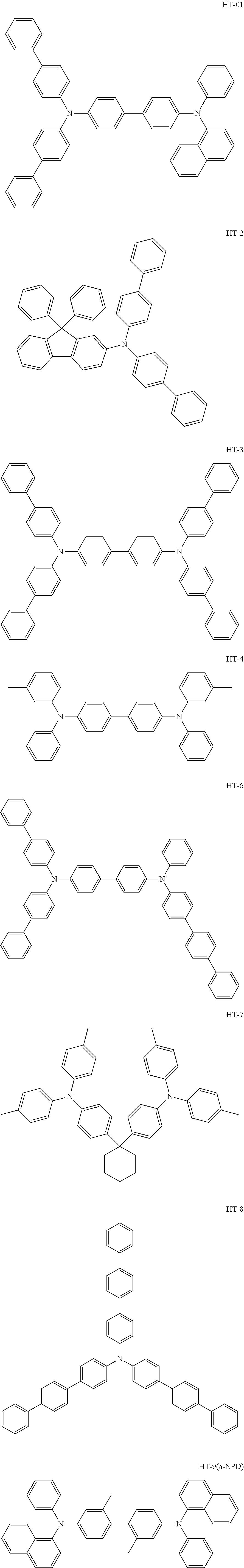

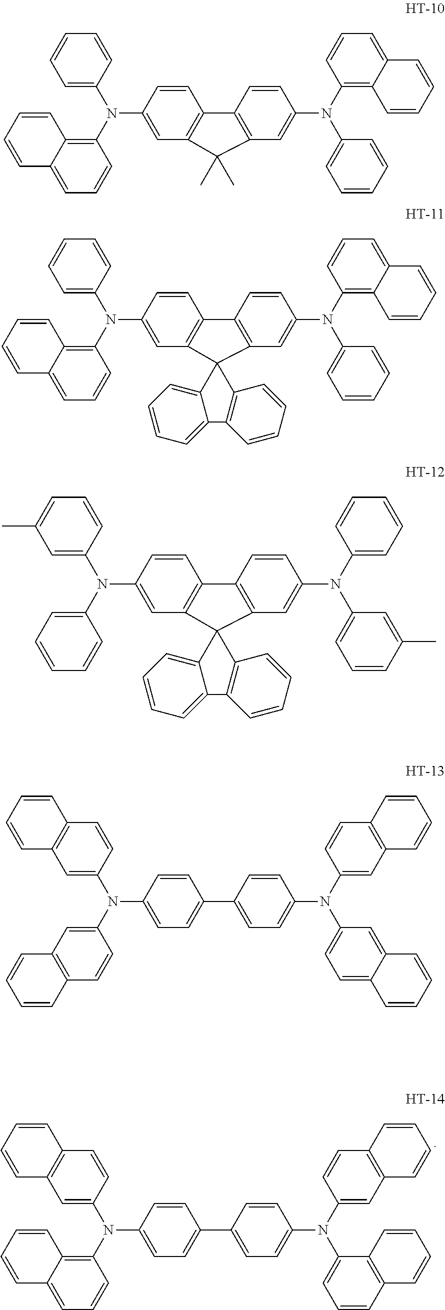

In one embodiment, the hole transport layer 321 is composed of HT-01.

In one embodiment, the hole adjustment layer 322 is composed of the compound of the present disclosure.

Optionally, the hole injection layer 310 is further arranged between the anode 100 and the hole transport layer 321 to enhance the capability of injecting holes into the hole transport layer 321. The hole injection layer 310 may be selected from benzidine derivatives, starburst arylamine compounds, phthalocyanine derivatives, or other materials, which are not particularly limited in the present disclosure. The material of the hole injection layer 310 is, for example, selected from the following compounds or any combination thereof:

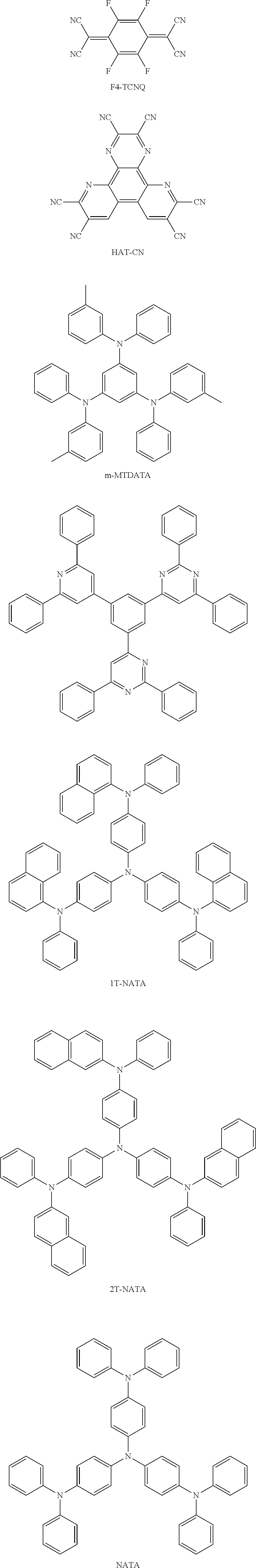

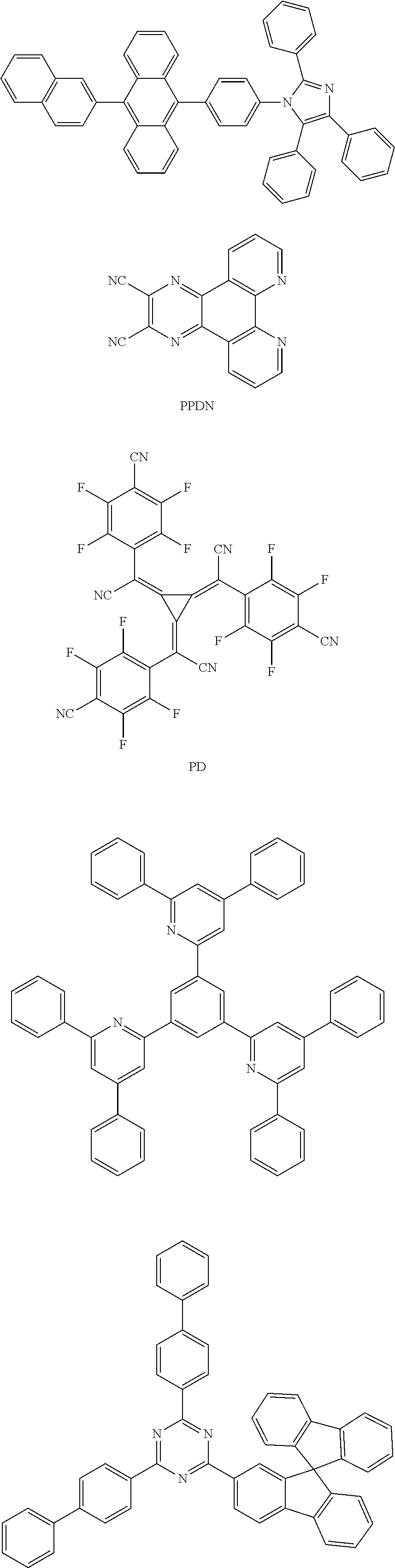

In one embodiment of the present disclosure, the hole injection layer 310 is composed of HAT-CN.

Optionally, the organic luminescent layer 330 may be composed of a single luminescent material, or include a host material and a guest material. Optionally, the organic luminescent layer 330 is composed of a host material and a guest material, holes injected into the organic luminescent layer 330 and electrons injected into the organic luminescent layer 330 can recombine in the organic luminescent layer 330 to form excitons, the excitons transfer energy to the host material, and the host material transfers energy to the guest material, so that the guest material can emit light.

The host material of the organic luminescent layer 330 may include metal chelating compounds, diphenyl vinyl derivatives, aromatic amine derivatives, dibenzofuran derivatives, or other types of materials. The host material of the organic luminescent layer 330 may be a compound or a combination of two or more compounds. Optionally, the host material includes the aromatic amine compound of the present disclosure.

The guest material of the organic luminescent layer 330 may be a compound having a condensed aryl ring or derivatives thereof, a compound having a heteroaryl ring or derivatives thereof, aromatic amine derivatives, or other materials, which are not particularly limited in the present disclosure. The guest material is also known as a doping material or dopant. According to the type of luminescence, the dopant may include fluorescent dopants and phosphorescent dopants. Specific examples of the phosphorescent dopants include, but are not limited to:

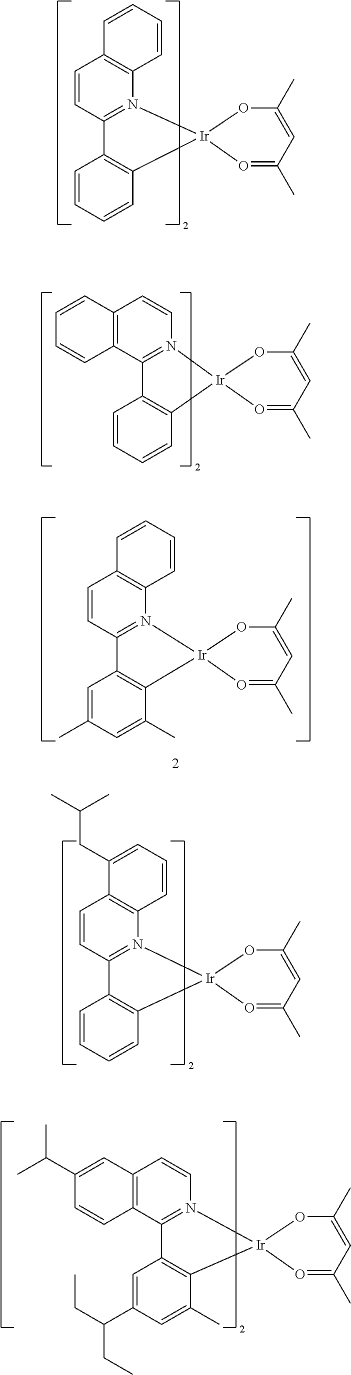

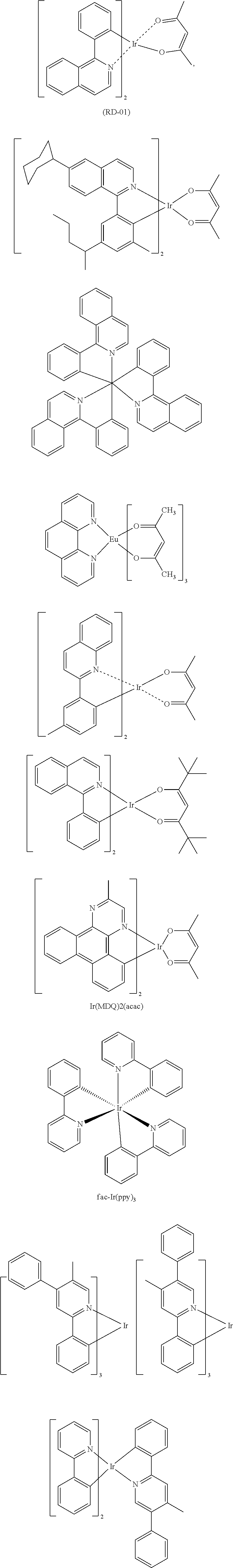

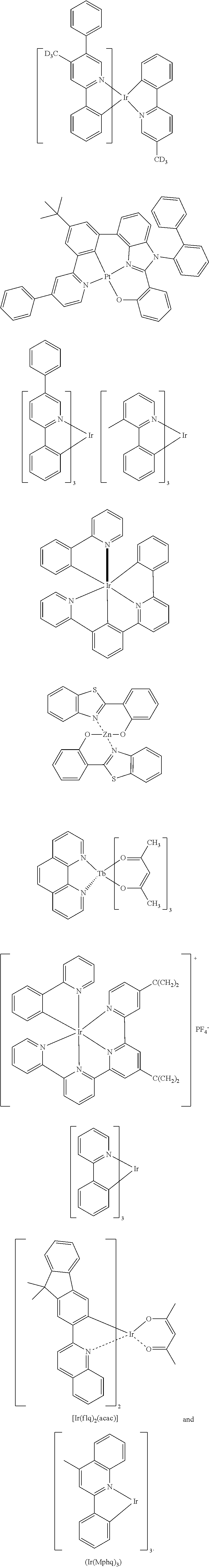

In one embodiment of the present disclosure, the organic electroluminescent device is a red organic electroluminescent device. In a more specific embodiment, the host material of the organic luminescent layer 330 includes RH-01

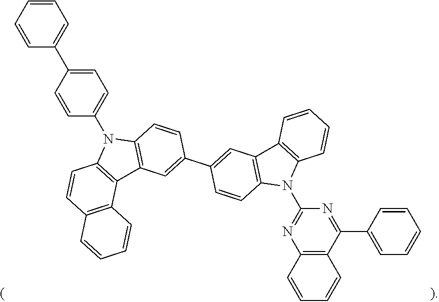 [SMILES: c1ccc(-c2ccc(-n3c4ccc(-c5ccc6c(c5)c5ccccc5n6-c5nc(-c6ccccc6)c6ccccc6n5)cc4c4c5ccccc5ccc43)cc2)cc1]

The guest material may be, for example, RD-01.

The hole blocking layer 340 may be of a single-layer structure or a multi-layer structure, which may include one or more hole blocking materials.

In a more specific embodiment, the material of the hole blocking layer 340 is HB-01

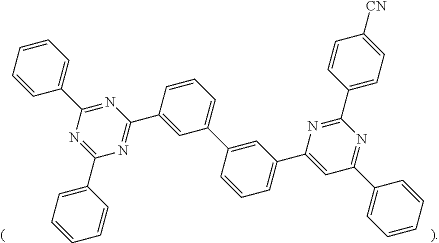 [SMILES: N#Cc1ccc(-c2nc(-c3ccccc3)cc(-c3cccc(-c4cccc(-c5nc(-c6ccccc6)nc(-c6ccccc6)n5)c4)c3)n2)cc1]

The electron transport layer 350 may be of a single-layer structure or a multi-layer structure, which may include one or more electron transport materials, and the electron transport materials may be selected from but are not limited to LiQ, benzimidazole derivatives, oxadiazole derivatives, quinoxaline derivatives, or other electron transport materials, which are not particularly limited in the present disclosure. The material of the electronic transport layer 350 includes but is not limited to the following compounds:

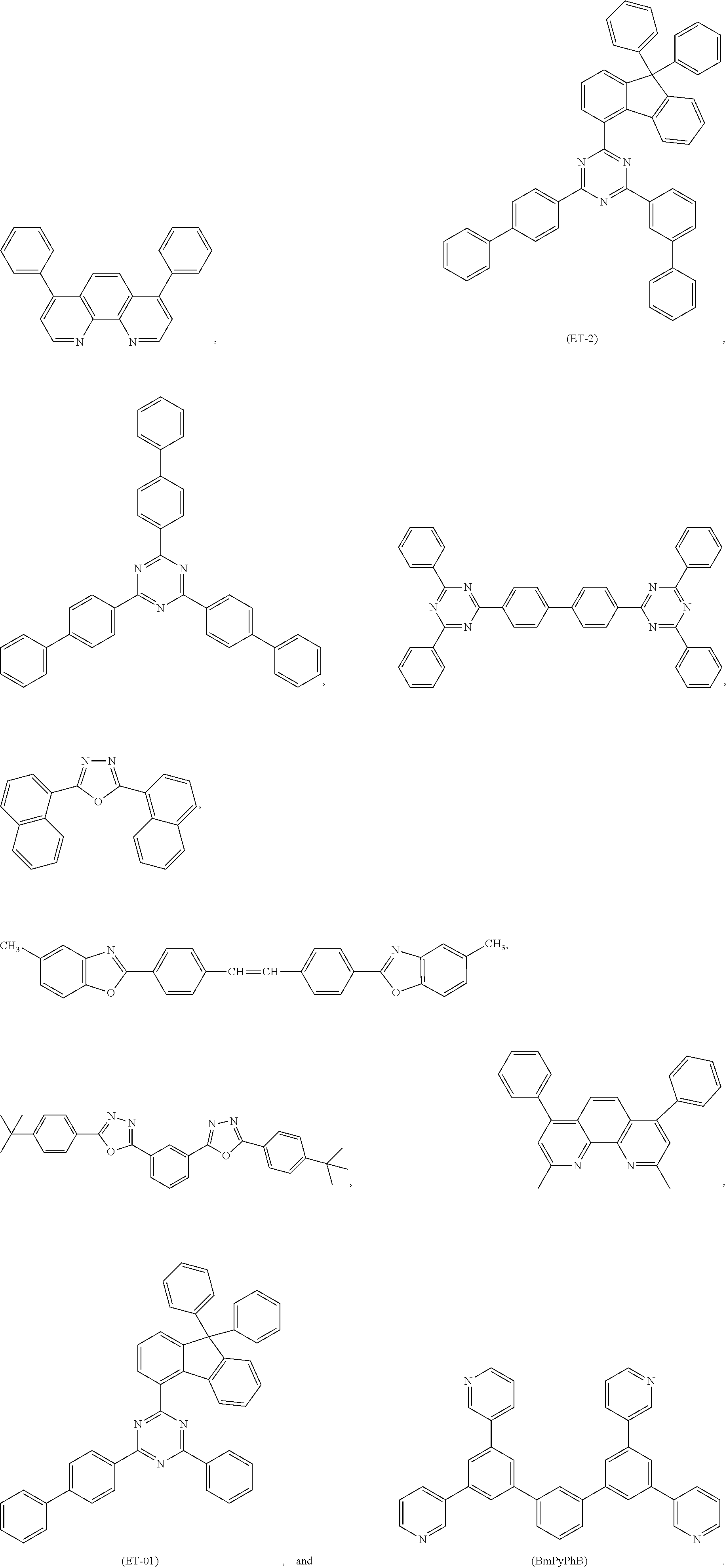

In one embodiment of the present disclosure, the electron transport layer 350 is composed of ET-01 and LiQ.

In the present disclosure, the cathode 200 includes a cathode material that facilitates electron injection into the functional layer and has a small work function. Specific examples of the cathode material include, but are not limited to, metals such as magnesium, calcium, sodium, potassium, titanium, indium, yttrium, lithium, gadolinium, aluminum, silver, tin, and lead, or alloys thereof; or multi-layer materials such as LiF/Al, Liq/Al, LiO₂/Al, LiF/Ca, LiF/Al, and BaF₂/Ca. A metal electrode containing magnesium and silver is preferably included as the cathode.

Optionally, the electron injection layer 360 is further arranged between the cathode 200 and the electron transport layer 350 to enhance the capability of injecting electrons into the electron transport layer 350. The electron injection layer 360 may include an inorganic material such as an alkali metal sulfide and an alkali metal halide, or may include a complex compound of an alkali metal and an organic matter. In one embodiment of the present disclosure, the electron injection layer 360 includes ytterbium (Yb).

A third aspect of the present disclosure provides an electronic apparatus, including the organic electroluminescent device described in the second aspect of the present disclosure.

According to one embodiment, as shown in FIG. 2, the provided electronic apparatus is an electronic apparatus 400, which includes the aforementioned organic electroluminescent device. The electronic apparatus 400 may be, for example, a display apparatus, a lighting apparatus, an optical communication apparatus, or other types of electronic apparatuses, such as but not limited to a computer screen, a mobile phone screen, a television, electronic paper, an emergency lamp, or an optical module.

Synthesis methods for the aromatic amine compound of the present disclosure will be described in detail below in conjunction with synthesis examples, but the present disclosure is not limited in any way.

### Examples of Synthesis

Those skilled in the art should recognize that the chemical reactions described in the present disclosure can be used for suitably preparing many aromatic amine compounds of the present disclosure, and other methods for preparing the compounds of the present disclosure are considered to fall within the scope of the present disclosure. For example, the synthesis of those non-exemplified compounds according to the present disclosure can be successfully accomplished by those skilled in the art by modifying methods, such as appropriately protecting interfering groups, using other known reagents in addition to those described in the present disclosure, or making some routine modifications on the reaction conditions. The compounds for the synthesis methods, not mentioned in the present disclosure, are all raw materials obtained through commercial channels.

### 1. Synthesis of Intermediate IM-A1

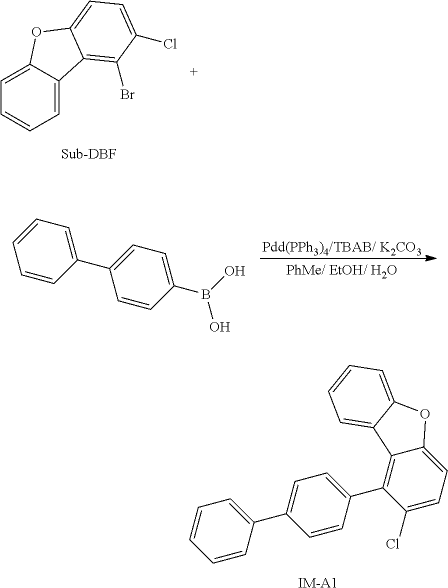

Under nitrogen atmosphere, Sub-DBF (70.0 g, 248.64 mmol), 4-biphenylboronic acid (51.7 g, 261.07 mmol), potassium carbonate (75.6 g, 547.01 mmol), toluene (600 mL), ethanol (300 mL), and water (150 mL) were added to a reaction flask, stirred, and heated to 50° C. to 60° C., tetrakis(triphenylphosphine)palladium (5.75 g, 4.97 mmol) and tetrabutylammonium bromide (TBAB) (16.0 g, 49.73 mmol) were quickly added, then the heating was continued to 70° C. to 75° C., and a reaction was carried out for 12 h with reflux reaction. The reaction solution was cooled and extracted with dichloromethane, the separated organic phase was washed with water until neutral, the organic phase was dried, filtrated, and the filtrate was concentrated in vacuum to obtain a crude product. The crude product was recrystallized with a mixed solvent of ethyl acetate and n-hexane until LC>98%, and a white solid IM-A1 was obtained after drying (63.8 g, yield 72.3%).

### 2. Synthesis of Intermediate IM-A2

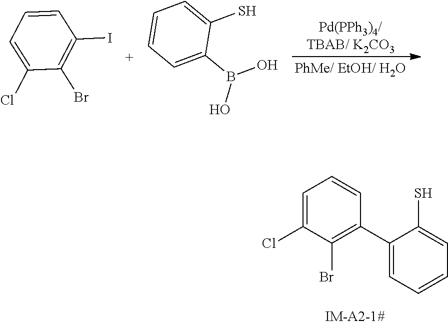

(1) Under nitrogen atmosphere, 2-bromo-1-chloro-3-iodobenzene (110.0 g, 346.62 mmol), o-mercaptophenylboronic acid (53.4 g, 346.62 mmol), potassium carbonate (105.4 g, 762.57 mmol), toluene (1000 mL), ethanol (400 mL), and water (200 mL) were added to a reaction flask, stirred, and heated to 50° C. to 60° C., tetrakis(triphenylphosphine)palladium (8.0 g, 6.93 mmol) and tetrabutylammonium bromide (TBAB) (22.35 g, 69.32 mmol) were quickly added, then the heating was continued to 70° C. to 75° C., and a reaction was carried out for 12 h with reflux reaction. The reaction solution was cooled and extracted with dichloromethane, the separated organic phase was washed with water until neutral, the organic phase was dried, filtrated, and the filtrate was concentrated in vacuum to obtain a crude product. The crude product was recrystallized with a mixed solvent of ethyl acetate and petroleum ether (volume ratio 1:3) until LC>98%, and a white solid IM-A2-1 # was obtained after drying (68.0 g, yield 65.5%).

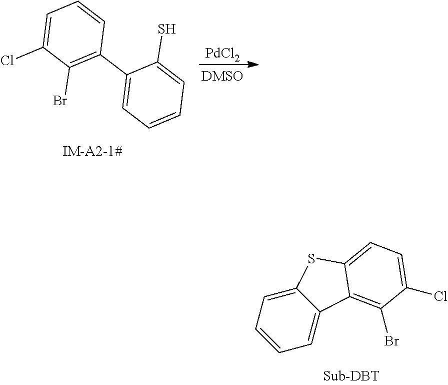

(2) Under nitrogen atmosphere, the IM-A2-1 #(67.0 g, mmol), palladium dichloride (1.98 g, 11.18 mmol), and dimethyl sulfoxide (600 mL) were added to a reaction flask, stirred, and heated to 140° C., and a reaction was carried out for 12 hours with reflux reaction, the reaction was stopped, and the reaction solution was cooled to 80° C., poured into 1200 mL of deionized water, stirred for 15 minutes, and stood for 1 hour. A large amount of solid was precipitated and filtered, the filter cake was washed 3 times with 300 mL of deionized water, and the obtained crude product was recrystallized with ethyl acetate and n-hexane to obtain a white solid Sub-DBT (59.4 g, yield 89.4%).

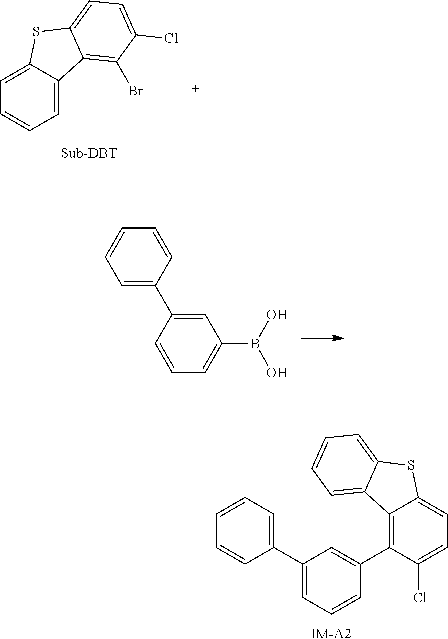

(3) Intermediate IM-A2 was synthesized using the same method as intermediate IM-A1, except that Sub-DBT was used instead of Sub-DBF and 3-biphenylboronic acid was used instead of 4-biphenylboronic acid, while other conditions were unchanged. Intermediate IM-A2 was obtained (20.2 g, yield 66.4%).

### 3. Synthesis of Intermediates IM-A3 to IM-A20, IM-A32, and IM-A33

Intermediates IM-A3 to IM-A20, IM-A32, and IM-A33 listed in Table 1 were synthesized with reference to the method for IM-A1. The differences were that raw material 1 was used instead of Sub-DBF, and raw material 2 was used instead of 4-biphenylboronic acid. The main raw materials used, the synthesized intermediates and the yields thereof were shown in Table 1.

TABLE 1

| Inter- | | | | |
|---|---|---|---|---|
| medi- | | | | Yield/ |
| ate | Raw material 1 | Raw material 2 | Product | % |
| IM- A3 | 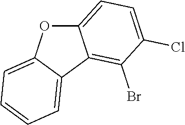 [SMILES: Clc1ccc2oc3ccccc3c2c1Br] | 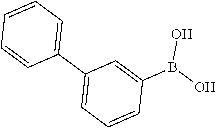 [SMILES: OB(O)c1cccc(-c2ccccc2)c1] | 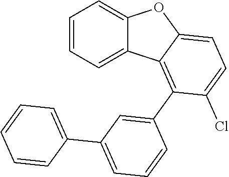 [SMILES: Clc1ccc2oc3ccccc3c2c1-c1cccc(-c2ccccc2)c1] | 69.2 |
| IM- A4 | 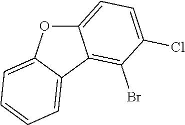 [SMILES: Clc1ccc2oc3ccccc3c2c1Br] | 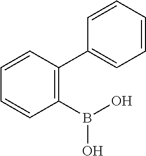 [SMILES: OB(O)c1ccccc1-c1ccccc1] | 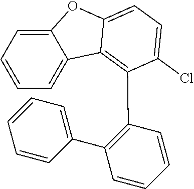 [SMILES: Clc1ccc2oc3ccccc3c2c1-c1ccccc1-c1ccccc1] | 66.0 |
| IM- A5 | 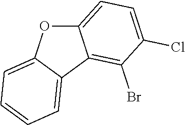 [SMILES: Clc1ccc2oc3ccccc3c2c1Br] | 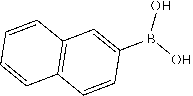 [SMILES: OB(O)c1ccc2ccccc2c1] | 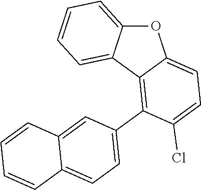 [SMILES: Clc1ccc2oc3ccccc3c2c1-c1ccc2ccccc2c1] | 72.5 |
| IM- A6 | 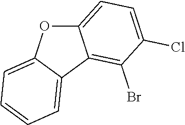 [SMILES: Clc1ccc2oc3ccccc3c2c1Br] | 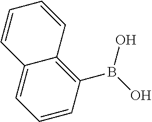 [SMILES: OB(O)c1cccc2ccccc12] | 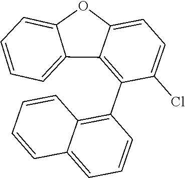 [SMILES: Clc1ccc2oc3ccccc3c2c1-c1cccc2ccccc12] | 65.4 |
| IM- A7 | 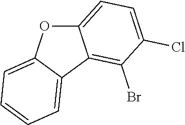 [SMILES: Clc1ccc2oc3ccccc3c2c1Br] | 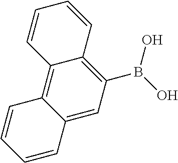 [SMILES: OB(O)c1cc2ccccc2c2ccccc12] | 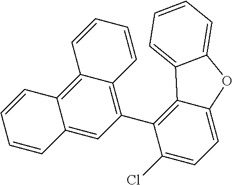 [SMILES: Clc1ccc2oc3ccccc3c2c1-c1cc2ccccc2c2ccccc12] | 71.7 |
| IM- A8 | 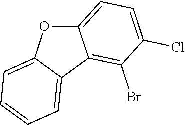 [SMILES: Clc1ccc2oc3ccccc3c2c1Br] | 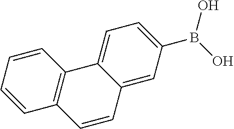 [SMILES: OB(O)c1ccc2c(ccc3ccccc32)c1] | 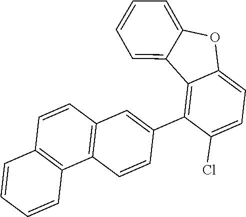 [SMILES: Clc1ccc2oc3ccccc3c2c1-c1ccc2c(ccc3ccccc32)c1] | 70.2 |
| IM- A9 | 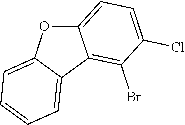 [SMILES: Clc1ccc2oc3ccccc3c2c1Br] | 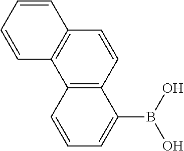 [SMILES: OB(O)c1cccc2c1ccc1ccccc12] | 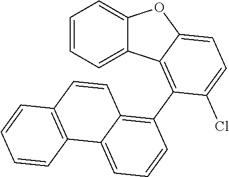 [SMILES: Clc1ccc2oc3ccccc3c2c1-c1cccc2c1ccc1ccccc12] | 63.0 |
| IM- A10 | 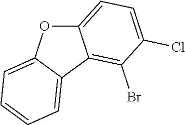 [SMILES: Clc1ccc2oc3ccccc3c2c1Br] | 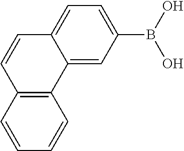 [SMILES: OB(O)c1ccc2ccc3ccccc3c2c1] | 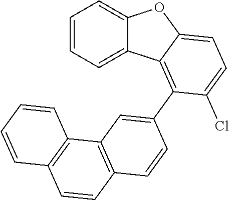 [SMILES: Clc1ccc2oc3ccccc3c2c1-c1ccc2ccc3ccccc3c2c1] | 71.2 |
| IM- A11 | 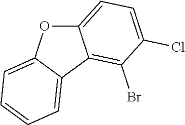 [SMILES: Clc1ccc2oc3ccccc3c2c1Br] | 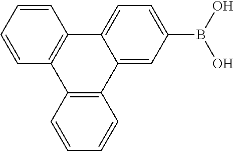 [SMILES: OB(O)c1ccc2c3ccccc3c3ccccc3c2c1] | 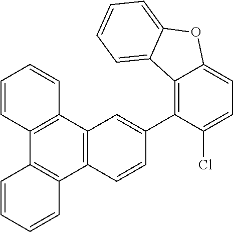 [SMILES: Clc1ccc2oc3ccccc3c2c1-c1ccc2c3ccccc3c3ccccc3c2c1] | 66.2 |
| IM- A12 | 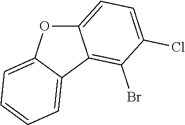 [SMILES: Clc1ccc2oc3ccccc3c2c1Br] | 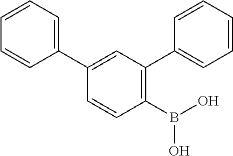 [SMILES: OB(O)c1ccc(-c2ccccc2)cc1-c1ccccc1] CAS:364050-45-3 | 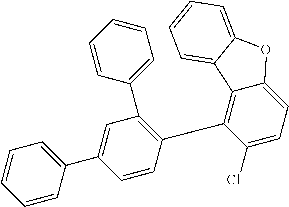 [SMILES: Clc1ccc2oc3ccccc3c2c1-c1ccc(-c2ccccc2)cc1-c1ccccc1] | 65.4 |
| IM- A13 | 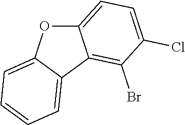 [SMILES: Clc1ccc2oc3ccccc3c2c1Br] | 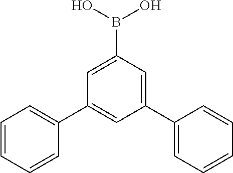 [SMILES: OB(O)c1cc(-c2ccccc2)cc(-c2ccccc2)c1] | 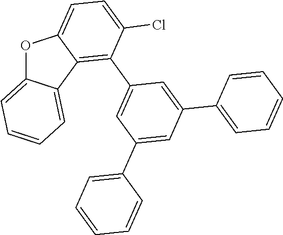 [SMILES: Clc1ccc2oc3ccccc3c2c1-c1cc(-c2ccccc2)cc(-c2ccccc2)c1] | 70.1 |
| IM- A14 | 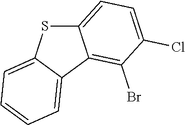 [SMILES: Clc1ccc2sc3ccccc3c2c1Br] | 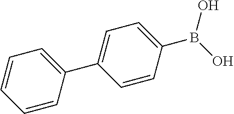 [SMILES: OB(O)c1ccc(-c2ccccc2)cc1] | 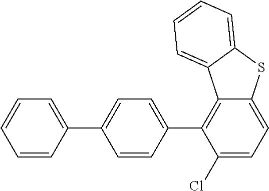 [SMILES: Clc1ccc2sc3ccccc3c2c1-c1ccc(-c2ccccc2)cc1] | 71.6 |
| IM- A15 | 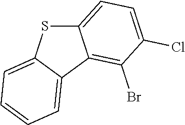 [SMILES: Clc1ccc2sc3ccccc3c2c1Br] | 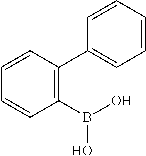 [SMILES: OB(O)c1ccccc1-c1ccccc1] | 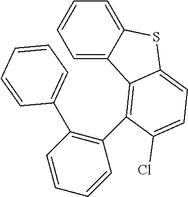 [SMILES: Clc1ccc2sc3ccccc3c2c1-c1ccccc1-c1ccccc1] | 67.0 |
| IM- A16 | 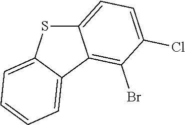 [SMILES: Clc1ccc2sc3ccccc3c2c1Br] | 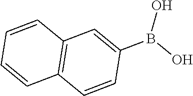 [SMILES: OB(O)c1ccc2ccccc2c1] | 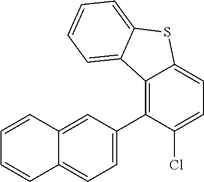 [SMILES: Clc1ccc2sc3ccccc3c2c1-c1ccc2ccccc2c1] | 71.1 |
| IM- A17 | 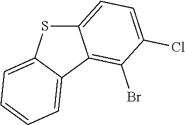 [SMILES: Clc1ccc2sc3ccccc3c2c1Br] | 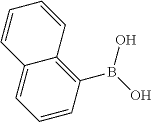 [SMILES: OB(O)c1cccc2ccccc12] | 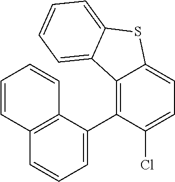 [SMILES: Clc1ccc2sc3ccccc3c2c1-c1cccc2ccccc12] | 66.3 |
| IM- A18 | 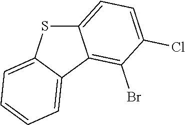 [SMILES: Clc1ccc2sc3ccccc3c2c1Br] | 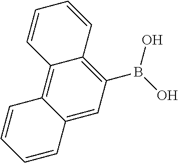 [SMILES: OB(O)c1cc2ccccc2c2ccccc12] | 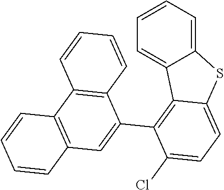 [SMILES: Clc1ccc2sc3ccccc3c2c1-c1cc2ccccc2c2ccccc12] | 64.5 |
| IM- A19 | 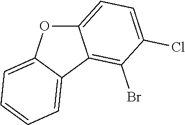 [SMILES: Clc1ccc2oc3ccccc3c2c1Br] | 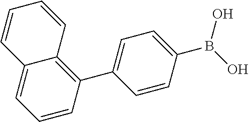 [SMILES: OB(O)c1ccc(-c2cccc3ccccc23)cc1] | 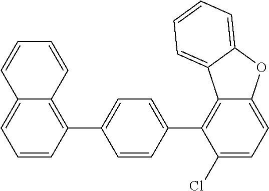 [SMILES: Clc1ccc2oc3ccccc3c2c1-c1ccc(-c2cccc3ccccc23)cc1] | 72.1 |
| IM- A20 | 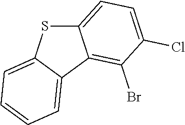 [SMILES: Clc1ccc2sc3ccccc3c2c1Br] | 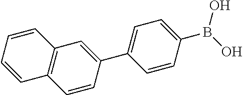 [SMILES: OB(O)c1ccc(-c2ccc3ccccc3c2)cc1] | 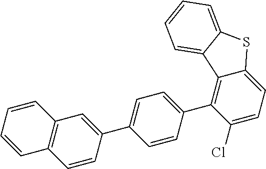 [SMILES: Clc1ccc2sc3ccccc3c2c1-c1ccc(-c2ccc3ccccc3c2)cc1] | 70.9 |
| IM- A32 | 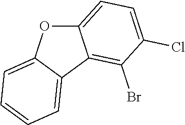 [SMILES: Clc1ccc2oc3ccccc3c2c1Br] | 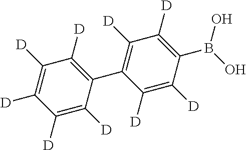 [SMILES: [2H]c1c([2H])c([2H])c(-c2c([2H])c([2H])c(B(O)O)c([2H])c2[2H])c([2H])c1[2H]] | 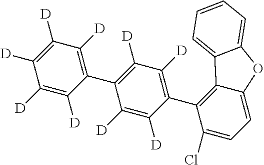 [SMILES: [2H]c1c([2H])c([2H])c(-c2c([2H])c([2H])c(-c3c(Cl)ccc4oc5ccccc5c34)c([2H])c2[2H])c([2H])c1[2H]] | 67.5 |
| IM- A33 | 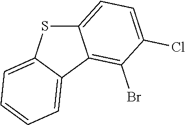 [SMILES: Clc1ccc2sc3ccccc3c2c1Br] | 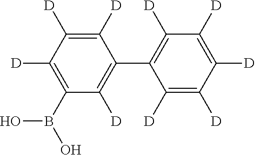 [SMILES: [2H]c1c([2H])c([2H])c(-c2c([2H])c([2H])c([2H])c(B(O)O)c2[2H])c([2H])c1[2H]] | 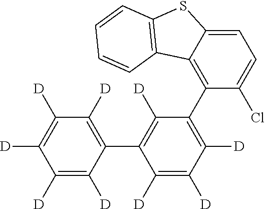 [SMILES: [2H]c1c([2H])c([2H])c(-c2c([2H])c([2H])c([2H])c(-c3c(Cl)ccc4sc5ccccc5c34)c2[2H])c([2H])c1[2H]] | 65.3 |

### 4. Synthesis of Intermediate IM-A21

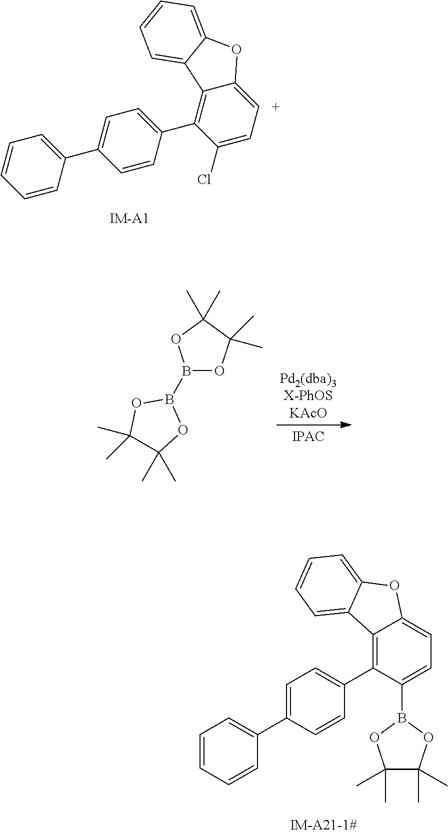

(1) Under nitrogen atmosphere, the intermediate IM-A1 (20.0 g, 56.37 mmol), bisdiboron (15.7 g, 62.00 mmol), tris(dibenzylideneacetone)dipalladium (1.03 g, 1.13 mmol), 2-dicyclohexylphosphine-2′,4′,6′-triisopropylbiphenyl (1.07 g, 2.25 mmol), and potassium acetate (8.3 g, 84.55 mmol) were added to a reaction flask, isopropyl acetate (IPAC, 180 mL) was added, and the solution was heated to 85° C. to 90° C. under nitrogen atmosphere with stirring for 24 hours; then the solution was cooled to room temperature, a product was precipitated, the product was filtered and washed until neutral, the obtained product was dissolved in toluene and separated by a silica gel chromatography column, the solvent was then evaporated in vacuum, and the product was purified by recrystallization with toluene to obtain intermediate IM-A21-1 #(16.9 g, yield 67.2%).

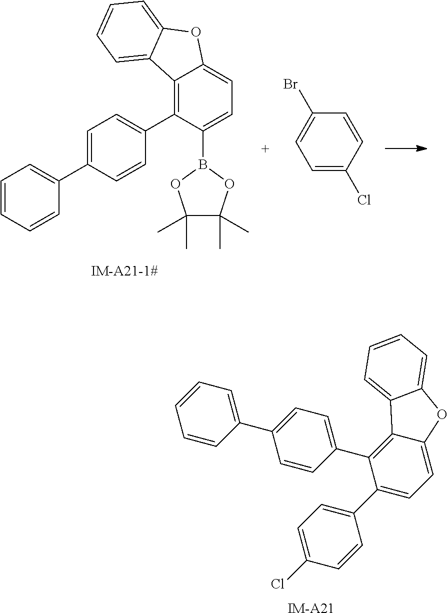

(2) The intermediate IM-A21-1 #(16.0 g, 35.85 mmol), p-chlorobromobenzene (6.9 g, 35.85 mmol), tetrabutylammonium bromide (TBAB, 0.46 g, 1.43 mmol), potassium carbonate (10.9 g, 78.86 mmol), toluene (120 mL), ethanol (45 mL), and water (30 mL) were added into a 250 mL three-necked flask, stirred, charged with nitrogen for protection, and heated to 50° C. to 60° C., and tetrakis(triphenylphosphine) palladium (0.83 g, 0.72 mmol) was quickly added. After addition, the heating was continued to 70° C. to 75° C., and a reaction was carried out for 12 h with reflux reaction, the reaction solution was cooled to room temperature, an organic phase was extracted with dichloromethane, and the obtained organic phase was washed with water until neutral, the organic phase was dried, filtrated, and the filtrate was concentrated in vacuum to obtain a crude product. The product was recrystallized with a mixed solvent of ethyl acetate and n-hexane (volume ratio of 1:2). Finally, white solid intermediate IM-A21 was obtained after drying (11.0 g, yield 71.2%).

### 5. Synthesis of Intermediates IM-A22 to IM-A31

Intermediates IM-Ay listed in Table 2 were synthesized with reference to the method for IM-A21. The differences were that IM-Ax was used instead of IM-A1 to synthesize a corresponding product IM-Ay-1 #firstly; and then raw material 3 was used instead of p-chlorobromobenzene and reacted with the IM-Ay-1 # to obtain a product IM-Ay. The main raw materials used, the synthesized intermediates and the yields thereof were shown in Table 2.

TABLE 2

| | Raw | | | Raw | | |
|---|---|---|---|---|---|---|
| Intermediate | material IM- | Product IM-Ay- | Yield/ | material | Product IM- | Yield/ |
| number | Ax | 1# | % | 3 | Ay | % |
| IM-A22 | 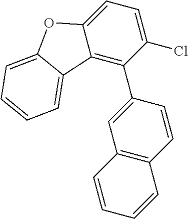 [SMILES: Clc1ccc2oc3ccccc3c2c1-c1ccc2ccccc2c1] | 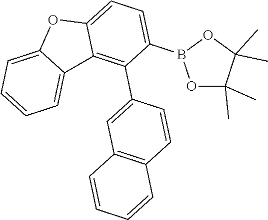 [SMILES: CC1(C)OB(c2ccc3oc4ccccc4c3c2-c2ccc3ccccc3c2)OC1(C)C] | 68.4 | 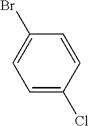 | 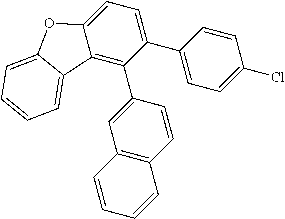 [SMILES: Clc1ccc(-c2ccc3oc4ccccc4c3c2-c2ccc3ccccc3c2)cc1] | 72.5 |
| IM-A23 | 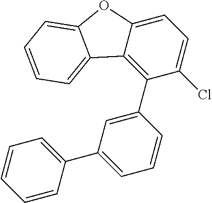 [SMILES: Clc1ccc2oc3ccccc3c2c1-c1cccc(-c2ccccc2)c1] | 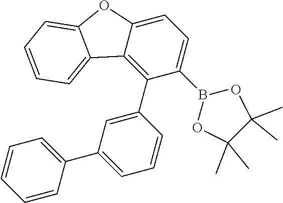 [SMILES: CC1(C)OB(c2ccc3oc4ccccc4c3c2-c2cccc(-c3ccccc3)c2)OC1(C)C] | 68.5 | 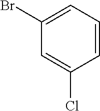 | 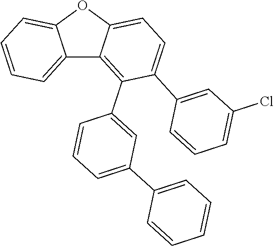 [SMILES: Clc1cccc(-c2ccc3oc4ccccc4c3c2-c2cccc(-c3ccccc3)c2)c1] | 74.1 |
| IM-A24 | 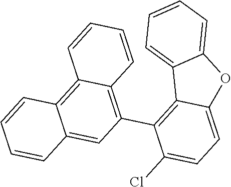 [SMILES: Clc1ccc2oc3ccccc3c2c1-c1cc2ccccc2c2ccccc12] | 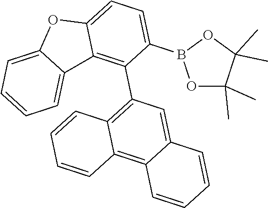 [SMILES: CC1(C)OB(c2ccc3oc4ccccc4c3c2-c2cc3ccccc3c3ccccc23)OC1(C)C] | 60.9 | 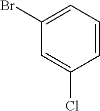 | 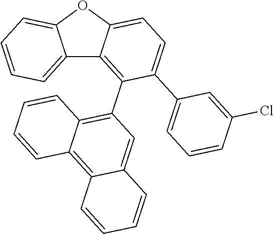 [SMILES: Clc1cccc(-c2ccc3oc4ccccc4c3c2-c2cc3ccccc3c3ccccc23)c1] | 68.9 |
| IM-A25 | 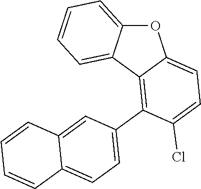 [SMILES: Clc1ccc2oc3ccccc3c2c1-c1ccc2ccccc2c1] | 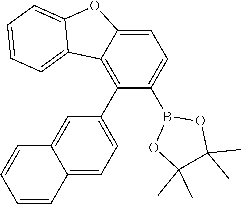 [SMILES: CC1(C)OB(c2ccc3oc4ccccc4c3c2-c2ccc3ccccc3c2)OC1(C)C] | 68.4 | 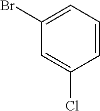 | 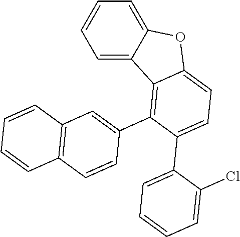 [SMILES: Clc1ccccc1-c1ccc2oc3ccccc3c2c1-c1ccc2ccccc2c1] | 67.7 |
| IM-A26 | 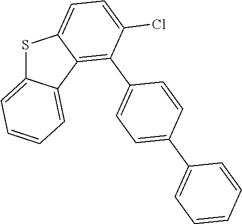 [SMILES: Clc1ccc2sc3ccccc3c2c1-c1ccc(-c2ccccc2)cc1] | 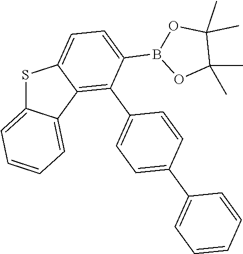 [SMILES: CC1(C)OB(c2ccc3sc4ccccc4c3c2-c2ccc(-c3ccccc3)cc2)OC1(C)C] | 68.5 | 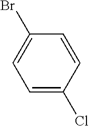 | 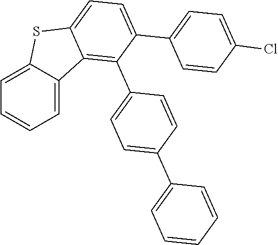 [SMILES: Clc1ccc(-c2ccc3sc4ccccc4c3c2-c2ccc(-c3ccccc3)cc2)cc1] | 72.9 |
| IM-A27 | 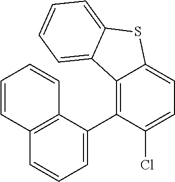 [SMILES: Clc1ccc2sc3ccccc3c2c1-c1cccc2ccccc12] | 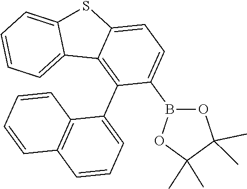 [SMILES: CC1(C)OB(c2ccc3sc4ccccc4c3c2-c2cccc3ccccc23)OC1(C)C] | 61.9 | 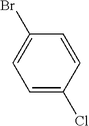 | 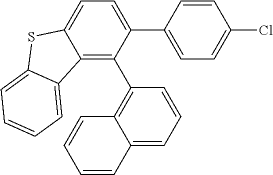 [SMILES: Clc1ccc(-c2ccc3sc4ccccc4c3c2-c2cccc3ccccc23)cc1] | 70.5 |
| IM-A28 | 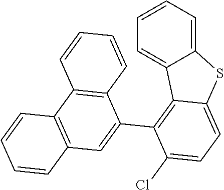 [SMILES: Clc1ccc2sc3ccccc3c2c1-c1cc2ccccc2c2ccccc12] | 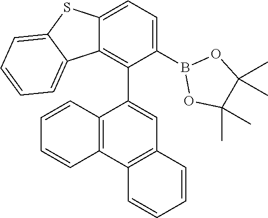 [SMILES: CC1(C)OB(c2ccc3sc4ccccc4c3c2-c2cc3ccccc3c3ccccc23)OC1(C)C] | 60.3 | 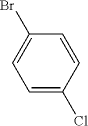 | 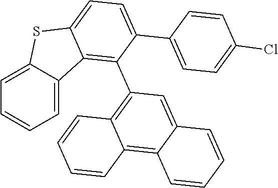 [SMILES: Clc1ccc(-c2ccc3sc4ccccc4c3c2-c2cc3ccccc3c3ccccc23)cc1] | 66.1 |
| IM-A29 | 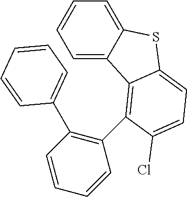 [SMILES: Clc1ccc2sc3ccccc3c2c1-c1ccccc1-c1ccccc1] | 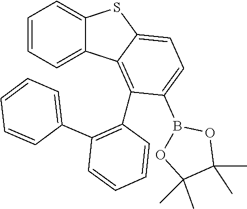 [SMILES: CC1(C)OB(c2ccc3sc4ccccc4c3c2-c2ccccc2-c2ccccc2)OC1(C)C] | 58.7 | 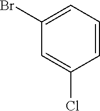 | 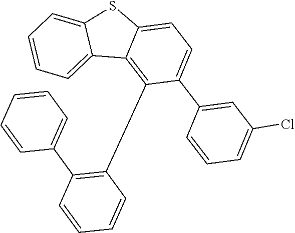 [SMILES: Clc1cccc(-c2ccc3sc4ccccc4c3c2-c2ccccc2-c2ccccc2)c1] | 64.6 |
| IM-A30 | 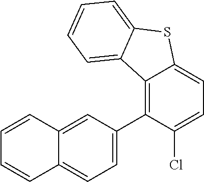 [SMILES: Clc1ccc2sc3ccccc3c2c1-c1ccc2ccccc2c1] | 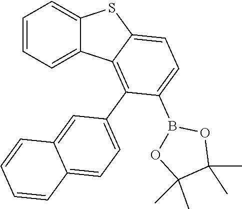 [SMILES: CC1(C)OB(c2ccc3sc4ccccc4c3c2-c2ccc3ccccc3c2)OC1(C)C] | 65.9 | 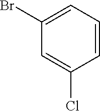 | 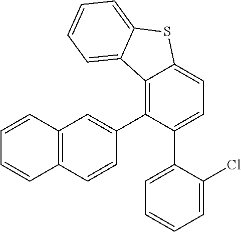 [SMILES: Clc1ccccc1-c1ccc2sc3ccccc3c2c1-c1ccc2ccccc2c1] | 62.3 |
| IM-A31 | 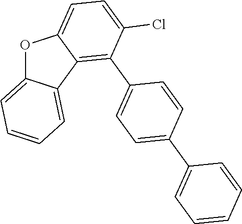 [SMILES: Clc1ccc2oc3ccccc3c2c1-c1ccc(-c2ccccc2)cc1] | 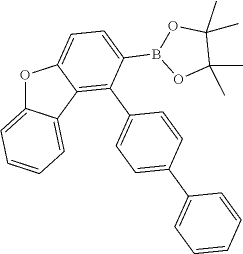 [SMILES: CC1(C)OB(c2ccc3oc4ccccc4c3c2-c2ccc(-c3ccccc3)cc2)OC1(C)C] | 68.5 | 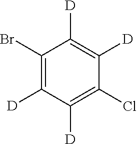 [SMILES: [2H]c1c([2H])c(Br)c([2H])c([2H])c1Cl] | 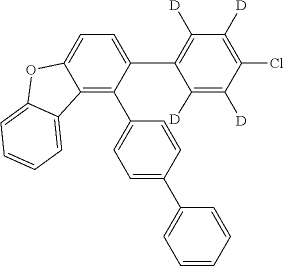 [SMILES: [2H]c1c([2H])c(-c2ccc3oc4ccccc4c3c2-c2ccc(-c3ccccc3)cc2)c([2H])c([2H])c1Cl] | 67.0 |

### 6. Synthesis of Intermediate IM-B1

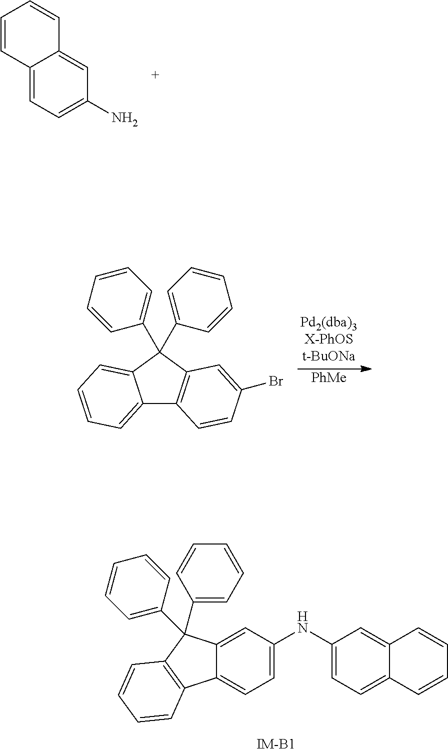 [SMILES: Nc1ccc2ccccc2c1 ; Brc1ccc2c(c1)C(c1ccccc1)(c1ccccc1)c1ccccc1-2 ; "PhMe" ; c1ccc(C2(c3ccccc3)c3ccccc3-c3ccc(Nc4ccc5ccccc5c4)cc32)cc1]

Under nitrogen atmosphere, 2-bromo-9,9-diphenylfluorene (13.88 g, 34.92 mmol), 2-naphthylamine (5.0 g, 34.92 mmol), tris(dibenzylideneacetone)dipalladium (0.32 g, 0.35 mmol), 2-dicyclohexylphosphine-2′,6′-dimethoxybiphenyl (0.33 g, 0.70 mmol), sodium tert-butoxide (5.03 g, 52.38 mmol), and toluene (110 mL) were added to a reaction flask and heated to 108° C. under nitrogen atmosphere, and a reaction was carried out for 12 h with reflux reaction and stirring. Then the reaction solution was cooled to room temperature and washed with water, anhydrous magnesium sulfate was added for drying, the solution was filtered, and the solvent was removed from the filtrate in vacuum to obtain a yellow crude solid. The crude solid was purified by recrystallization with a toluene system to obtain IM-B1 (12.87 g, yield 80.2%).

### 7. Synthesis of Intermediates IM-B2 to IM-B40

IM-Bx was synthesized with reference to the method for IM-B1. The differences were that raw material 4 was used instead of 2-naphthylamine and raw material 5 was used instead of 2-bromo-9,9-diphenylfluorene. The main raw materials used, the synthesized intermediates and the yields thereof were shown in Table 3.

TABLE 3

| Intermediate IM-Bx | Raw material 4 | Raw material 5 | Product | Yield/% |
|---|---|---|---|---|
| IM-B2 | 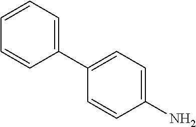 [SMILES: Nc1ccc(-c2ccccc2)cc1] | 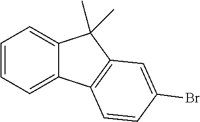 [SMILES: CC1(C)c2ccccc2-c2ccc(Br)cc21] | 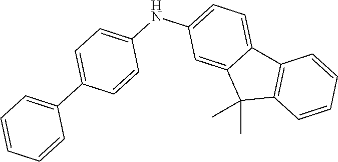 [SMILES: CC1(C)c2ccccc2-c2ccc(Nc3ccc(-c4ccccc4)cc3)cc21] | 83.1 |
| IM-B3 | 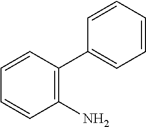 [SMILES: Nc1ccccc1-c1ccccc1] | 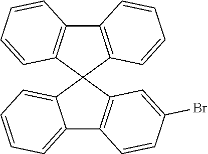 [SMILES: Brc1ccc2c(c1)C1(c3ccccc3-c3ccccc31)c1ccccc1-2] | 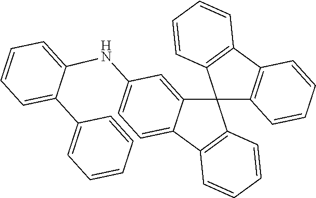 [SMILES: c1ccc(-c2ccccc2Nc2ccc3c(c2)C2(c4ccccc4-c4ccccc42)c2ccccc2-3)cc1] | 73.2 |
| IM-B4 | 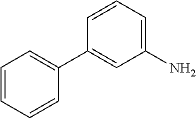 [SMILES: Nc1cccc(-c2ccccc2)c1] | 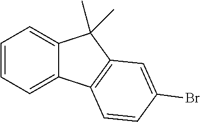 [SMILES: CC1(C)c2ccccc2-c2ccc(Br)cc21] | 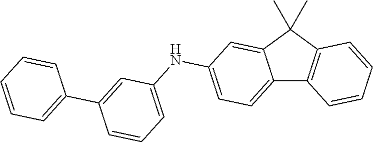 [SMILES: CC1(C)c2ccccc2-c2ccc(Nc3cccc(-c4ccccc4)c3)cc21] | 78.0 |
| IM-B5 | 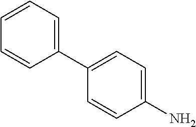 [SMILES: Nc1ccc(-c2ccccc2)cc1] | 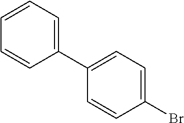 [SMILES: Brc1ccc(-c2ccccc2)cc1] | 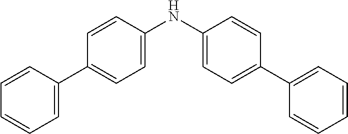 [SMILES: c1ccc(-c2ccc(Nc3ccc(-c4ccccc4)cc3)cc2)cc1] | 84.7 |
| IM-B6 | 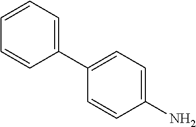 [SMILES: Nc1ccc(-c2ccccc2)cc1] | 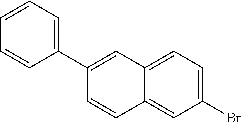 [SMILES: Brc1ccc2cc(-c3ccccc3)ccc2c1] | 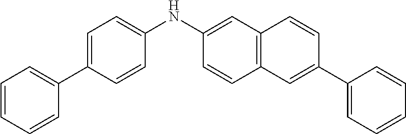 [SMILES: c1ccc(-c2ccc(Nc3ccc4cc(-c5ccccc5)ccc4c3)cc2)cc1] | 76.5 |
| IM-B7 | 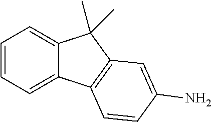 [SMILES: CC1(C)c2ccccc2-c2ccc(N)cc21] | 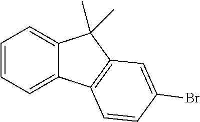 [SMILES: CC1(C)c2ccccc2-c2ccc(Br)cc21] | 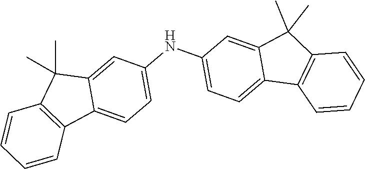 [SMILES: CC1(C)c2ccccc2-c2ccc(Nc3ccc4c(c3)C(C)(C)c3ccccc3-4)cc21] | 82.0 |
| IM-B8 | 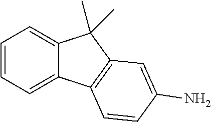 [SMILES: CC1(C)c2ccccc2-c2ccc(N)cc21] | 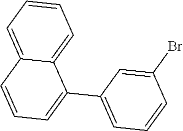 [SMILES: Brc1cccc(-c2cccc3ccccc23)c1] | 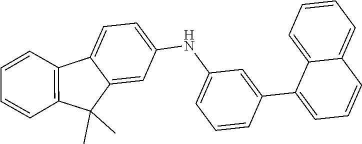 [SMILES: CC1(C)c2ccccc2-c2ccc(Nc3cccc(-c4cccc5ccccc45)c3)cc21] | 70.4 |
| IM-B9 | 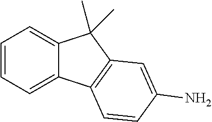 [SMILES: CC1(C)c2ccccc2-c2ccc(N)cc21] | 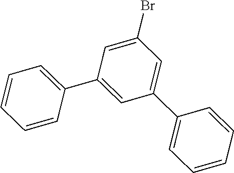 [SMILES: Brc1cc(-c2ccccc2)cc(-c2ccccc2)c1] | 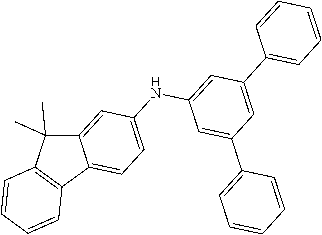 [SMILES: CC1(C)c2ccccc2-c2ccc(Nc3cc(-c4ccccc4)cc(-c4ccccc4)c3)cc21] | 69.2 |
| IM-B10 | 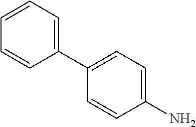 [SMILES: Nc1ccc(-c2ccccc2)cc1] | 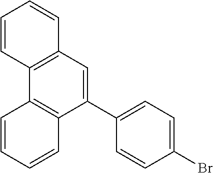 [SMILES: Brc1ccc(-c2cc3ccccc3c3ccccc23)cc1] | 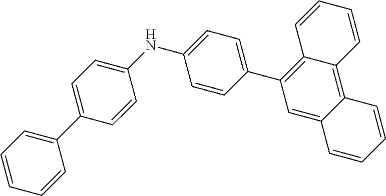 [SMILES: c1ccc(-c2ccc(Nc3ccc(-c4cc5ccccc5c5ccccc45)cc3)cc2)cc1] | 70.0 |
| IM-B11 | 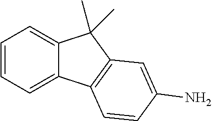 [SMILES: CC1(C)c2ccccc2-c2ccc(N)cc21] | 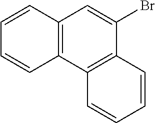 [SMILES: Brc1cc2ccccc2c2ccccc12] | 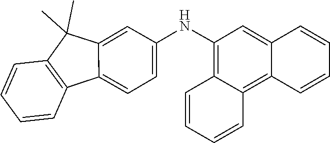 [SMILES: CC1(C)c2ccccc2-c2ccc(Nc3cc4ccccc4c4ccccc34)cc21] | 68.0 |
| IM-B12 | 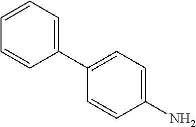 [SMILES: Nc1ccc(-c2ccccc2)cc1] | 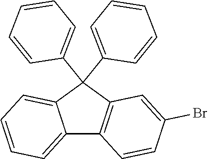 [SMILES: Brc1ccc2c(c1)C(c1ccccc1)(c1ccccc1)c1ccccc1-2] | 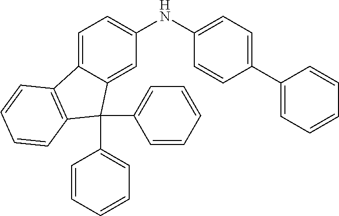 [SMILES: c1ccc(-c2ccc(Nc3ccc4c(c3)C(c3ccccc3)(c3ccccc3)c3ccccc3-4)cc2)cc1] | 71.7 |
| IM-B13 | 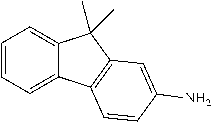 [SMILES: CC1(C)c2ccccc2-c2ccc(N)cc21] | 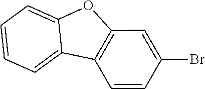 [SMILES: Brc1ccc2c(c1)oc1ccccc12] | 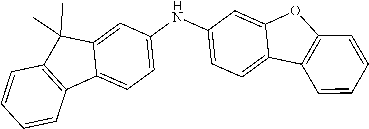 [SMILES: CC1(C)c2ccccc2-c2ccc(Nc3ccc4c(c3)oc3ccccc34)cc21] | 75.2 |
| IM-B14 | 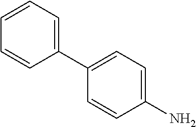 [SMILES: Nc1ccc(-c2ccccc2)cc1] | 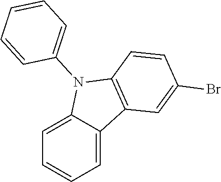 [SMILES: Brc1ccc2c(c1)c1ccccc1n2-c1ccccc1] | 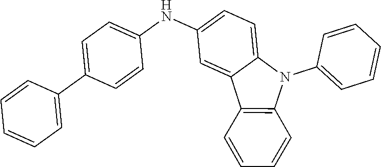 [SMILES: c1ccc(-c2ccc(Nc3ccc4c(c3)c3ccccc3n4-c3ccccc3)cc2)cc1] | 78.2 |
| IM-B15 | 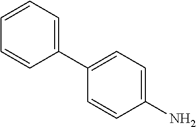 [SMILES: Nc1ccc(-c2ccccc2)cc1] | 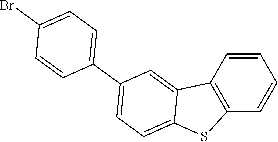 [SMILES: Brc1ccc(-c2ccc3sc4ccccc4c3c2)cc1] | 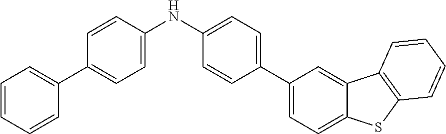 [SMILES: c1ccc(-c2ccc(Nc3ccc(-c4ccc5sc6ccccc6c5c4)cc3)cc2)cc1] | 75.8 |
| IM-B16 | 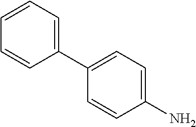 [SMILES: Nc1ccc(-c2ccccc2)cc1] | 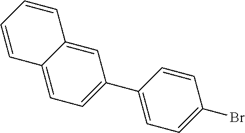 [SMILES: Brc1ccc(-c2ccc3ccccc3c2)cc1] | 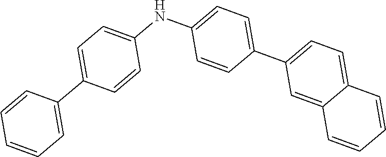 [SMILES: c1ccc(-c2ccc(Nc3ccc(-c4ccc5ccccc5c4)cc3)cc2)cc1] | 74.5 |
| IM-B17 | 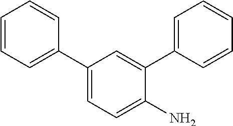 [SMILES: Nc1ccc(-c2ccccc2)cc1-c1ccccc1] | 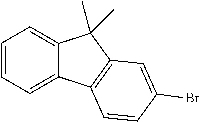 [SMILES: CC1(C)c2ccccc2-c2ccc(Br)cc21] | 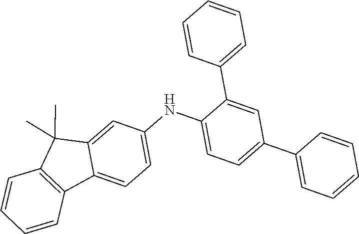 [SMILES: CC1(C)c2ccccc2-c2ccc(Nc3ccc(-c4ccccc4)cc3-c3ccccc3)cc21] | 70.5 |
| IM-B18 | 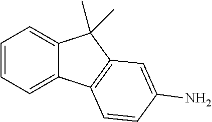 [SMILES: CC1(C)c2ccccc2-c2ccc(N)cc21] | 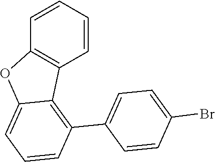 [SMILES: Brc1ccc(-c2cccc3oc4ccccc4c23)cc1] | 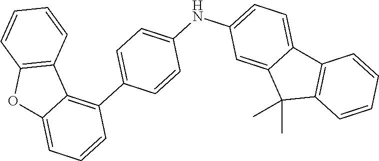 [SMILES: CC1(C)c2ccccc2-c2ccc(Nc3ccc(-c4cccc5oc6ccccc6c45)cc3)cc21] | 71.3 |
| IM-B19 | 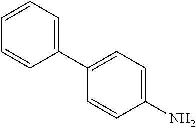 [SMILES: Nc1ccc(-c2ccccc2)cc1] | 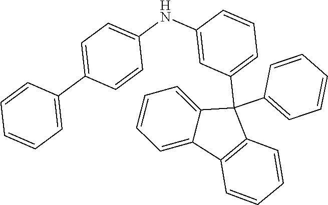 [SMILES: c1ccc(-c2ccc(Nc3cccc(C4(c5ccccc5)c5ccccc5-c5ccccc54)c3)cc2)cc1] | 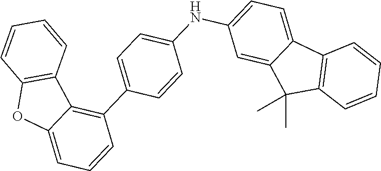 [SMILES: CC1(C)c2ccccc2-c2ccc(Nc3ccc(-c4cccc5oc6ccccc6c45)cc3)cc21] | 70.1 |
| IM-B20 | 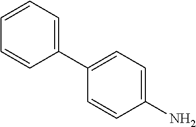 [SMILES: Nc1ccc(-c2ccccc2)cc1] | 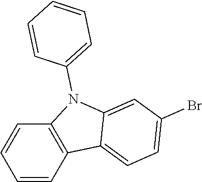 [SMILES: Brc1ccc2c3ccccc3n(-c3ccccc3)c2c1] | 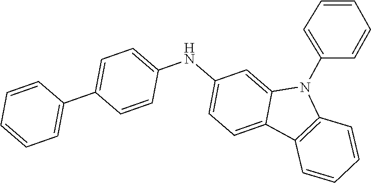 [SMILES: c1ccc(-c2ccc(Nc3ccc4c5ccccc5n(-c5ccccc5)c4c3)cc2)cc1] | 79.4 |
| IM-B21 | 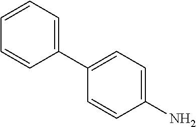 [SMILES: Nc1ccc(-c2ccccc2)cc1] | 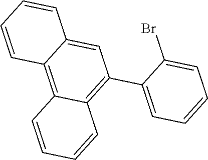 [SMILES: Brc1ccccc1-c1cc2ccccc2c2ccccc12] | 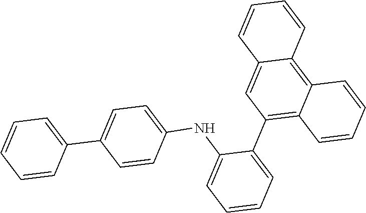 [SMILES: c1ccc(-c2ccc(Nc3ccccc3-c3cc4ccccc4c4ccccc34)cc2)cc1] | 68.5 |
| IM-B22 | 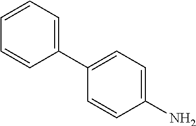 [SMILES: Nc1ccc(-c2ccccc2)cc1] | 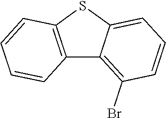 [SMILES: Brc1cccc2sc3ccccc3c12] | 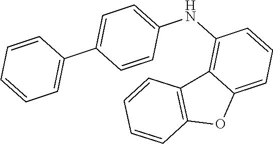 [SMILES: c1ccc(-c2ccc(Nc3cccc4oc5ccccc5c34)cc2)cc1] | 64.2 |
| IM-B23 | 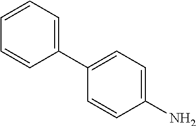 [SMILES: Nc1ccc(-c2ccccc2)cc1] | 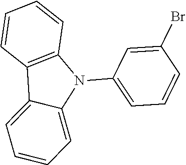 [SMILES: Brc1cccc(-n2c3ccccc3c3ccccc32)c1] | 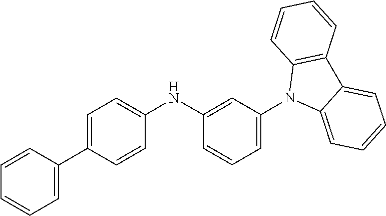 [SMILES: c1ccc(-c2ccc(Nc3cccc(-n4c5ccccc5c5ccccc54)c3)cc2)cc1] | 70.3 |
| IM-B24 | 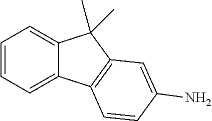 [SMILES: CC1(C)c2ccccc2-c2ccc(N)cc21] | 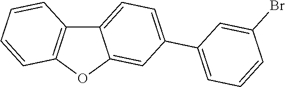 [SMILES: Brc1cccc(-c2ccc3c(c2)oc2ccccc23)c1] | 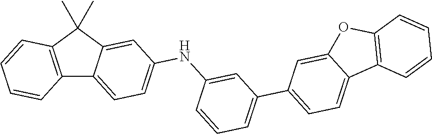 [SMILES: CC1(C)c2ccccc2-c2ccc(Nc3cccc(-c4ccc5c(c4)oc4ccccc45)c3)cc21] | 66.2 |
| IM-B25 | 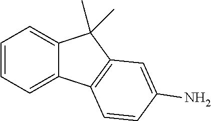 [SMILES: CC1(C)c2ccccc2-c2ccc(N)cc21] | 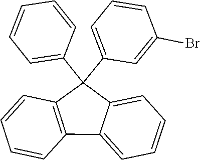 [SMILES: Brc1cccc(C2(c3ccccc3)c3ccccc3-c3ccccc32)c1] | 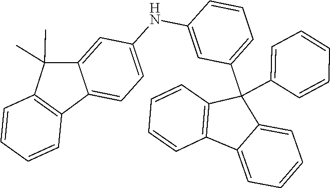 [SMILES: CC1(C)c2ccccc2-c2ccc(Nc3cccc(C4(c5ccccc5)c5ccccc5-c5ccccc54)c3)cc21] | 70.9 |
| IM-B26 | 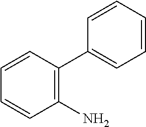 [SMILES: Nc1ccccc1-c1ccccc1] | 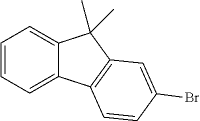 [SMILES: CC1(C)c2ccccc2-c2ccc(Br)cc21] | 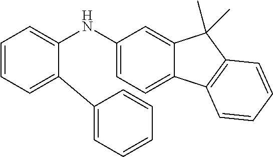 [SMILES: CC1(C)c2ccccc2-c2ccc(Nc3ccccc3-c3ccccc3)cc21] | 76.0 |
| IM-B27 | 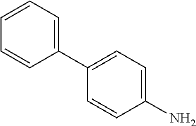 [SMILES: Nc1ccc(-c2ccccc2)cc1] | 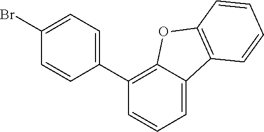 [SMILES: Brc1ccc(-c2cccc3c2oc2ccccc23)cc1] | 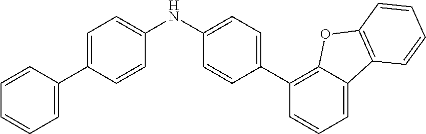 [SMILES: c1ccc(-c2ccc(Nc3ccc(-c4cccc5c4oc4ccccc45)cc3)cc2)cc1] | 74.7 |
| IM-B28 | 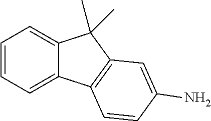 [SMILES: CC1(C)c2ccccc2-c2ccc(N)cc21] | 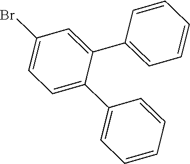 [SMILES: Brc1ccc(-c2ccccc2)c(-c2ccccc2)c1] | 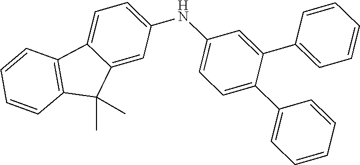 [SMILES: CC1(C)c2ccccc2-c2ccc(Nc3ccc(-c4ccccc4)c(-c4ccccc4)c3)cc21] | 69.3 |
| IM-B29 | 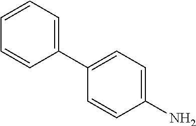 [SMILES: Nc1ccc(-c2ccccc2)cc1] | 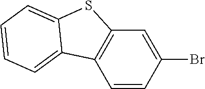 [SMILES: Brc1ccc2c(c1)sc1ccccc12] | 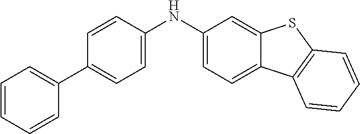 [SMILES: c1ccc(-c2ccc(Nc3ccc4c(c3)sc3ccccc34)cc2)cc1] | 78.9 |
| IM-B30 | 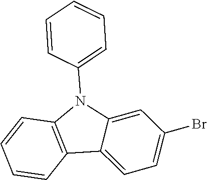 [SMILES: Brc1ccc2c3ccccc3n(-c3ccccc3)c2c1] | 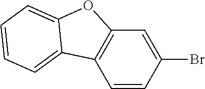 [SMILES: Brc1ccc2c(c1)oc1ccccc12] | 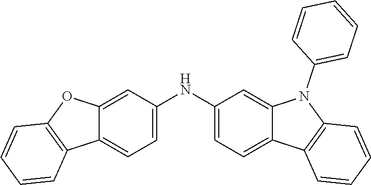 [SMILES: c1ccc(-n2c3ccccc3c3ccc(Nc4ccc5c(c4)oc4ccccc45)cc32)cc1] | 77.2 |
| IM-B31 | 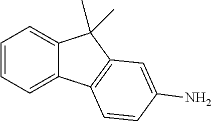 [SMILES: CC1(C)c2ccccc2-c2ccc(N)cc21] | 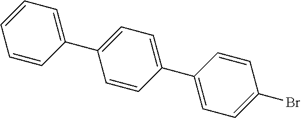 [SMILES: Brc1ccc(-c2ccc(-c3ccccc3)cc2)cc1] | 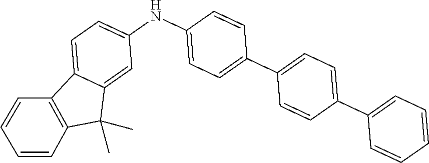 [SMILES: CC1(C)c2ccccc2-c2ccc(Nc3ccc(-c4ccc(-c5ccccc5)cc4)cc3)cc21] | 80.8 |
| IM-B32 | 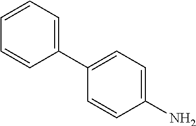 [SMILES: Nc1ccc(-c2ccccc2)cc1] | 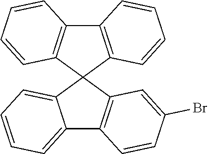 [SMILES: Brc1ccc2c(c1)C1(c3ccccc3-c3ccccc31)c1ccccc1-2] | 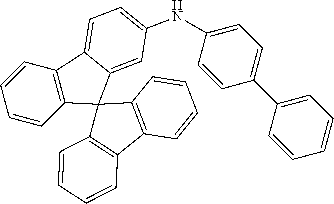 [SMILES: c1ccc(-c2ccc(Nc3ccc4c(c3)C3(c5ccccc5-c5ccccc53)c3ccccc3-4)cc2)cc1] | 79.4 |
| IM-B33 | 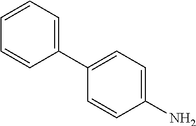 [SMILES: Nc1ccc(-c2ccccc2)cc1] | 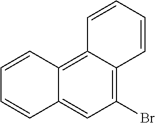 [SMILES: Brc1cc2ccccc2c2ccccc12] | 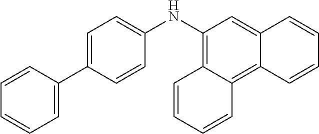 [SMILES: c1ccc(-c2ccc(Nc3cc4ccccc4c4ccccc34)cc2)cc1] | 74.0 |
| IM-B34 | 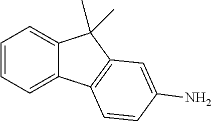 [SMILES: CC1(C)c2ccccc2-c2ccc(N)cc21] | 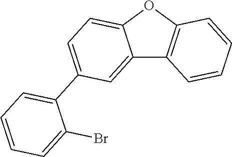 [SMILES: Brc1ccccc1-c1ccc2oc3ccccc3c2c1] | 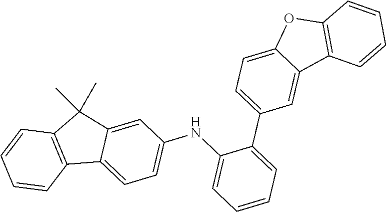 [SMILES: CC1(C)c2ccccc2-c2ccc(Nc3ccccc3-c3ccc4oc5ccccc5c4c3)cc21] | 63.5 |
| IM-B35 | 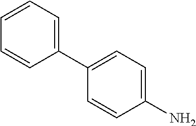 [SMILES: Nc1ccc(-c2ccccc2)cc1] | 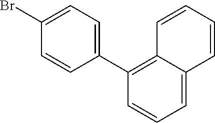 [SMILES: Brc1ccc(-c2cccc3ccccc23)cc1] | 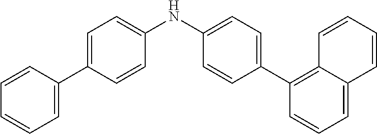 [SMILES: c1ccc(-c2ccc(Nc3ccc(-c4cccc5ccccc45)cc3)cc2)cc1] | 83.7 |
| IM-B36 | 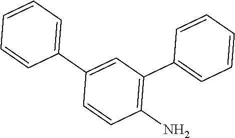 [SMILES: Nc1ccc(-c2ccccc2)cc1-c1ccccc1] | 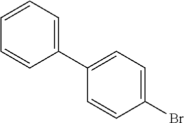 [SMILES: Brc1ccc(-c2ccccc2)cc1] | 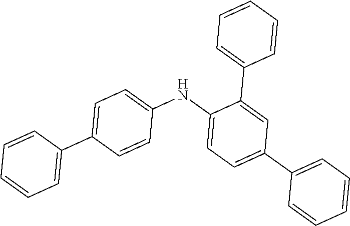 [SMILES: c1ccc(-c2ccc(Nc3ccc(-c4ccccc4)cc3-c3ccccc3)cc2)cc1] | 69.3 |
| IM-B37 | 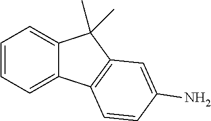 [SMILES: CC1(C)c2ccccc2-c2ccc(N)cc21] | 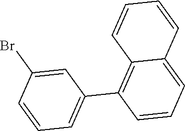 [SMILES: Brc1cccc(-c2cccc3ccccc23)c1] | 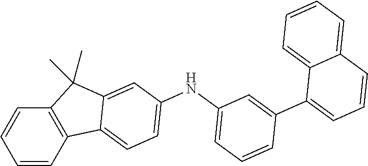 [SMILES: CC1(C)c2ccccc2-c2ccc(Nc3cccc(-c4cccc5ccccc45)c3)cc21] | 72.8 |
| IM-B38 | 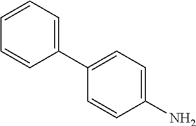 [SMILES: Nc1ccc(-c2ccccc2)cc1] | 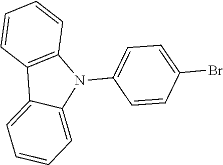 [SMILES: Brc1ccc(-n2c3ccccc3c3ccccc32)cc1] | 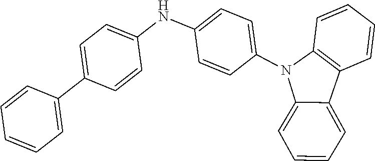 [SMILES: c1ccc(-c2ccc(Nc3ccc(-n4c5ccccc5c5ccccc54)cc3)cc2)cc1] | 76.6 |
| IM-B39 | 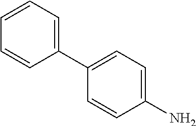 [SMILES: Nc1ccc(-c2ccccc2)cc1] | 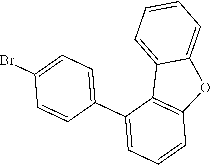 [SMILES: Brc1ccc(-c2cccc3oc4ccccc4c23)cc1] | 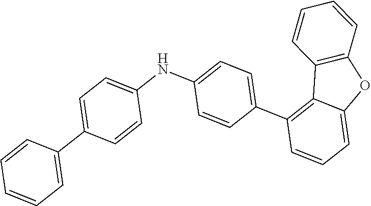 [SMILES: c1ccc(-c2ccc(Nc3ccc(-c4cccc5oc6ccccc6c45)cc3)cc2)cc1] | 73.5 |
| IM-B40 | 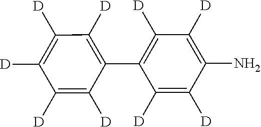 [SMILES: [2H]c1c([2H])c([2H])c(-c2c([2H])c([2H])c(N)c([2H])c2[2H])c([2H])c1[2H]] | 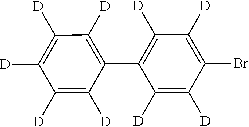 [SMILES: [2H]c1c([2H])c([2H])c(-c2c([2H])c([2H])c(Br)c([2H])c2[2H])c([2H])c1[2H]] | 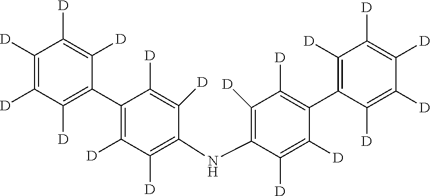 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] | 72.4 |

### 8. Synthesis Example 1: Synthesis of Compound 9

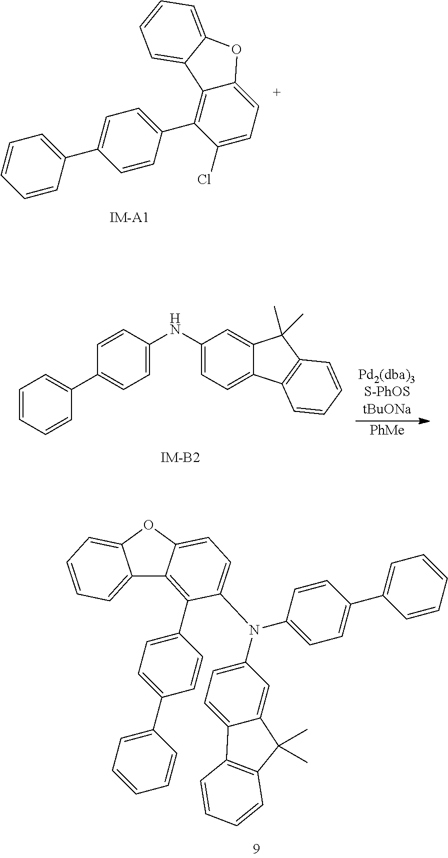

Under nitrogen atmosphere, the IM-A1 (5.0 g, 14.09 mmol), IM-B2 (5.1 g, 14.09 mmol), tris(dibenzylideneacetone)dipalladium (0.13 g, 0.14 mmol), 2-dicyclohexylphosphine-2′,6′-dimethoxybiphenyl (0.12 g, 0.28 mmol), sodium tert-butoxide (2.03 g, 21.14 mmol), and toluene (40 mL) were sequentially added to a round bottom flask, heated to 108° C., and stirred for 4 hours; then the reaction solution was cooled to room temperature and washed with water, magnesium sulfate was added for drying, the solution was filtered, the solvent was removed from the filtrate in vacuum to obtain a crude product, and the crude product was purified by recrystallization with toluene to obtain a white solid compound 9 (5.0 g, yield 52.2%). Mass spectrometry (m/z): m/z=680.3 [M+H]⁺.

Compounds listed in Table 4 were synthesized with reference to the method for compound 9. The differences were that raw material 6 was used instead of intermediate IM-A1 and raw material 7 was used instead of intermediate IM-B2. The main raw materials used, the synthesized compounds and the yields and mass spectra thereof were shown in Table 4.

TABLE 4

| Syn- | | | | | |
|---|---|---|---|---|---|
| the- | | | | | |
| sis | | | | | |
| ex- | | | | | |
| am- | | | | | |
| ple | | | | Yi- | Mass |
| num- | | | | eld/ | spec- |
| ber | Raw material 6 | Raw material 7 | Product | % | tra |
| 2 | 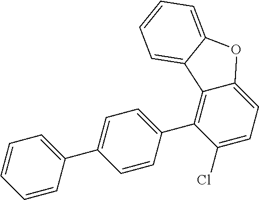 [SMILES: Clc1ccc2oc3ccccc3c2c1-c1ccc(-c2ccccc2)cc1] IM-A1 | 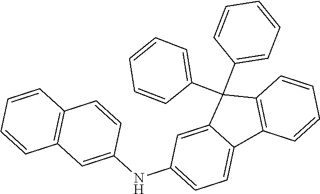 [SMILES: c1ccc(C2(c3ccccc3)c3ccccc3-c3ccc(Nc4ccc5ccccc5c4)cc32)cc1] IM-B1 | 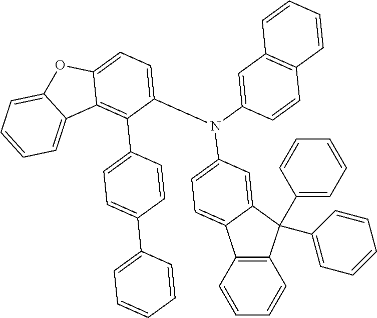 [SMILES: c1ccc(-c2ccc(-c3c(CN(c4ccc5c(c4)C(c4ccccc4)(c4ccccc4)c4ccccc4-5)c4ccc5ccccc5c4)ccc4oc5ccccc5c34)cc2)cc1] 5 | 39.3 | 778.3 |
| 3 | 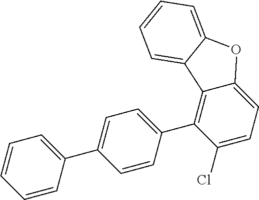 [SMILES: Clc1ccc2oc3ccccc3c2c1-c1ccc(-c2ccccc2)cc1] IM-A1 | 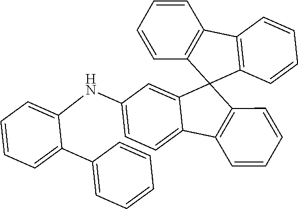 [SMILES: c1ccc(-c2ccccc2Nc2ccc3c(c2)C2(c4ccccc4-c4ccccc42)c2ccccc2-3)cc1] IM-B3 | 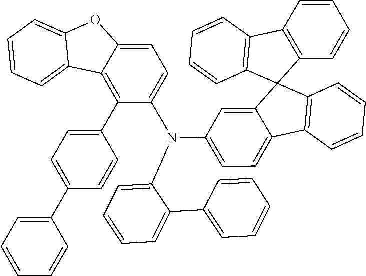 [SMILES: c1ccc(-c2ccc(-c3c(N(c4ccc5c(c4)C4(c6ccccc6-c6ccccc64)c4ccccc4-5)c4ccccc4-c4ccccc4)ccc4oc5ccccc5c34)cc2)cc1] 15 | 41.6 | 802.3 |
| 4 | 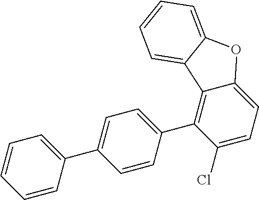 [SMILES: Clc1ccc2oc3ccccc3c2c1-c1ccc(-c2ccccc2)cc1] IM-A1 | 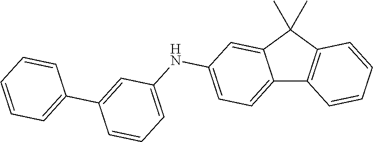 [SMILES: CC1(C)c2ccccc2-c2ccc(Nc3cccc(-c4ccccc4)c3)cc21] IM-B4 | 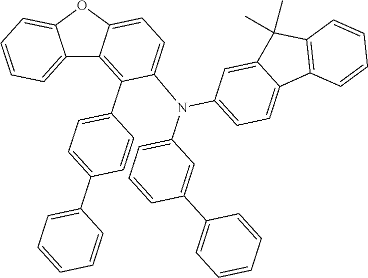 [SMILES: CC1(C)c2ccccc2-c2ccc(N(c3cccc(-c4ccccc4)c3)c3ccc4oc5ccccc5c4c3-c3ccc(-c4ccccc4)cc3)cc21] 19 | 48.6 | 680.3 |
| 5 | 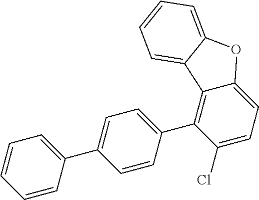 [SMILES: Clc1ccc2oc3ccccc3c2c1-c1ccc(-c2ccccc2)cc1] IM-A1 | 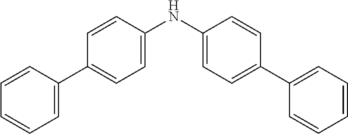 [SMILES: c1ccc(-c2ccc(Nc3ccc(-c4ccccc4)cc3)cc2)cc1] IM-B5 | 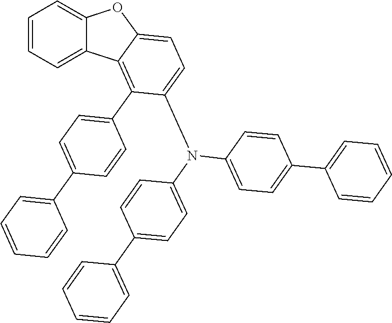 [SMILES: c1ccc(-c2ccc(-c3c(CN(c4ccc(-c5ccccc5)cc4)c4ccc(-c5ccccc5)cc4)ccc4oc5ccccc5c34)cc2)cc1] 21 | 38.3 | 640.2 |
| 6 | 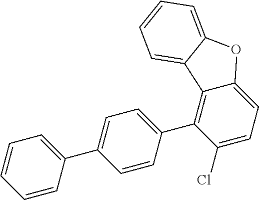 [SMILES: Clc1ccc2oc3ccccc3c2c1-c1ccc(-c2ccccc2)cc1] IM-A1 | 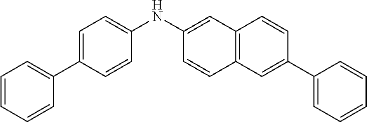 [SMILES: c1ccc(-c2ccc(Nc3ccc4cc(-c5ccccc5)ccc4c3)cc2)cc1] IM-B6 | 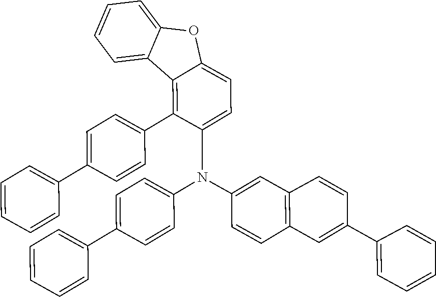 [SMILES: c1ccc(-c2ccc(-c3c(N(c4ccc(-c5ccccc5)cc4)c4ccc5cc(-c6ccccc6)ccc5c4)ccc4oc5ccccc5c34)cc2)cc1] 24 | 49.9 | 690.3 |
| 7 | 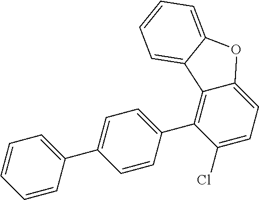 [SMILES: Clc1ccc2oc3ccccc3c2c1-c1ccc(-c2ccccc2)cc1] IM-A1 | 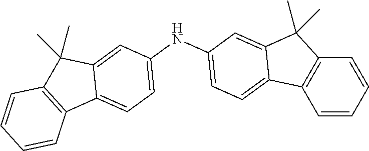 [SMILES: CC1(C)c2ccccc2-c2ccc(Nc3ccc4c(c3)C(C)(C)c3ccccc3-4)cc21] IM-B7 | 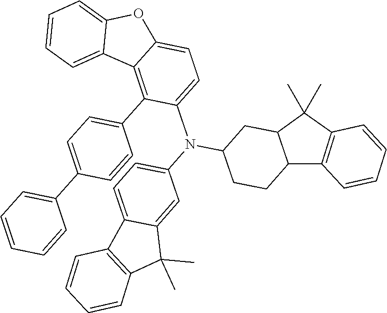 [SMILES: CC1(C)c2ccccc2-c2ccc(N(c3ccc4oc5ccccc5c4c3-c3ccc(-c4ccccc4)cc3)C3CCC4c5ccccc5C(C)(C)C4C3)cc21] 31 | 40.5 | 720.3 |
| 8 | 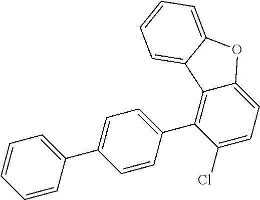 [SMILES: Clc1ccc2oc3ccccc3c2c1-c1ccc(-c2ccccc2)cc1] IM-A1 | 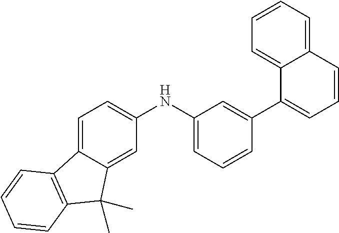 [SMILES: CC1(C)c2ccccc2-c2ccc(Nc3cccc(-c4cccc5ccccc45)c3)cc21] IM-B8 | 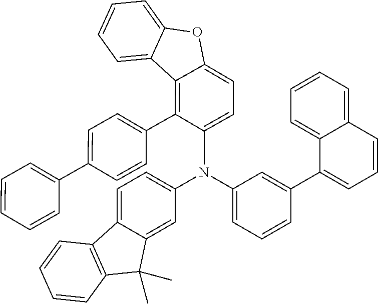 [SMILES: CC1(C)c2ccccc2-c2ccc(N(c3cccc(-c4cccc5ccccc45)c3)c3ccc4oc5ccccc5c4c3-c3ccc(-c4ccccc4)cc3)cc21] 37 | 39.9 | 730.3 |
| 9 | 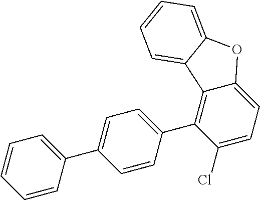 [SMILES: Clc1ccc2oc3ccccc3c2c1-c1ccc(-c2ccccc2)cc1] IM-A1 | 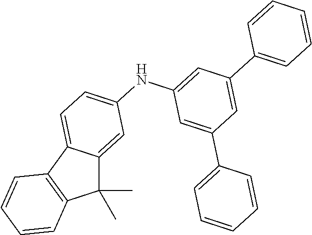 [SMILES: CC1(C)c2ccccc2-c2ccc(Nc3cc(-c4ccccc4)cc(-c4ccccc4)c3)cc21] IM-B9 | 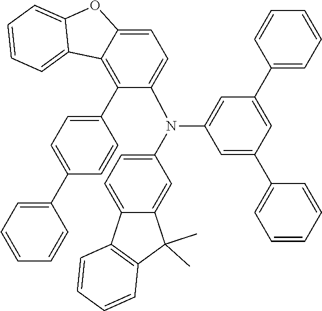 [SMILES: CC1(C)c2ccccc2-c2ccc(N(c3cc(-c4ccccc4)cc(-c4ccccc4)c3)c3ccc4oc5ccccc5c4c3-c3ccc(-c4ccccc4)cc3)cc21] 40 | 37.3 | 756.3 |
| 10 | 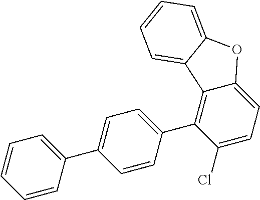 [SMILES: Clc1ccc2oc3ccccc3c2c1-c1ccc(-c2ccccc2)cc1] IM-A1 | 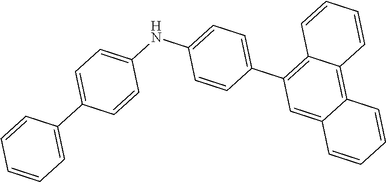 [SMILES: c1ccc(-c2ccc(Nc3ccc(-c4cc5ccccc5c5ccccc45)cc3)cc2)cc1] IM-B10 | 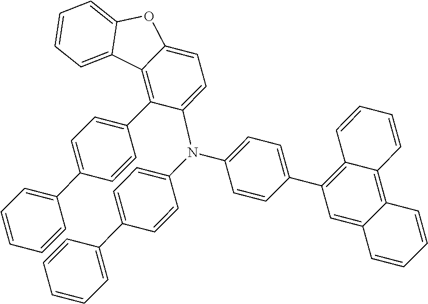 [SMILES: c1ccc(-c2ccc(-c3c(N(c4ccc(-c5ccccc5)cc4)c4ccc(-c5cc6ccccc6c6ccccc56)cc4)ccc4oc5ccccc5c34)cc2)cc1] 46 | 40.2 | 740.3 |
| 11 | 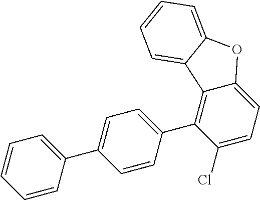 [SMILES: Clc1ccc2oc3ccccc3c2c1-c1ccc(-c2ccccc2)cc1] IM-A1 | 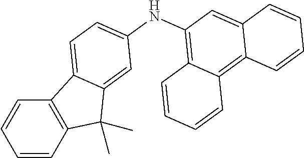 [SMILES: CC1(C)c2ccccc2-c2ccc(Nc3cc4ccccc4c4ccccc34)cc21] IM-B11 | 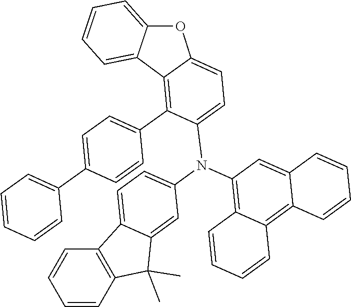 [SMILES: CC1(C)c2ccccc2-c2ccc(N(c3ccc4oc5ccccc5c4c3-c3ccc(-c4ccccc4)cc3)c3cc4ccccc4c4ccccc34)cc21] 53 | 42.5 | 704.3 |
| 12 | 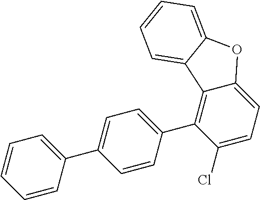 [SMILES: Clc1ccc2oc3ccccc3c2c1-c1ccc(-c2ccccc2)cc1] IM-A1 | 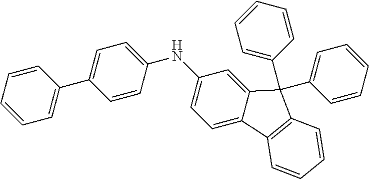 [SMILES: c1ccc(-c2ccc(Nc3ccc4c(c3)C(c3ccccc3)(c3ccccc3)c3ccccc3-4)cc2)cc1] IM-B12 | 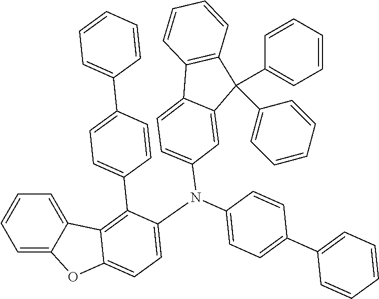 [SMILES: c1ccc(-c2ccc(-c3c(N(c4ccc(-c5ccccc5)cc4)c4ccc5c(c4)C(c4ccccc4)(c4ccccc4)c4ccccc4-5)ccc4oc5ccccc5c34)cc2)cc1] 58 | 49.0 | 804.3 |
| 13 | 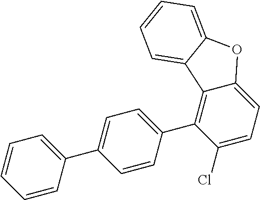 [SMILES: Clc1ccc2oc3ccccc3c2c1-c1ccc(-c2ccccc2)cc1] IM-A1 | 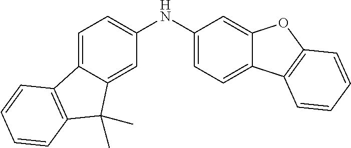 [SMILES: CC1(C)c2ccccc2-c2ccc(Nc3ccc4c(c3)oc3ccccc34)cc21] IM-B13 | 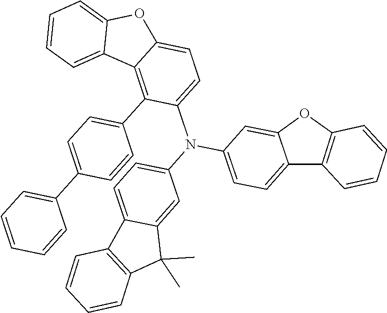 [SMILES: CC1(C)c2ccccc2-c2ccc(N(c3ccc4c(c3)oc3ccccc34)c3ccc4oc5ccccc5c4c3-c3ccc(-c4ccccc4)cc3)cc21] 64 | 40.0 | 694.3 |
| 14 | 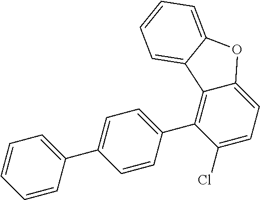 [SMILES: Clc1ccc2oc3ccccc3c2c1-c1ccc(-c2ccccc2)cc1] IM-A1 | 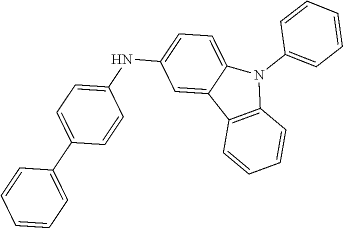 [SMILES: c1ccc(-c2ccc(Nc3ccc4c(c3)c3ccccc3n4-c3ccccc3)cc2)cc1] IM-B14 | 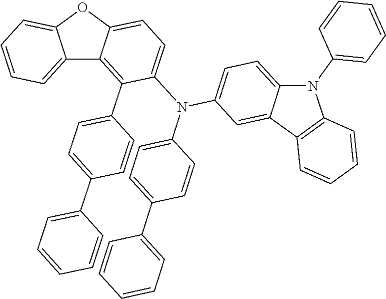 [SMILES: c1ccc(-c2ccc(-c3c(N(c4ccc(-c5ccccc5)cc4)c4ccc5c(c4)c4ccccc4n5-c4ccccc4)ccc4oc5ccccc5c34)cc2)cc1] 66 | 41.4 | 729.3 |
| 15 | 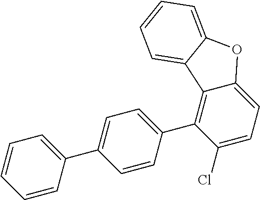 [SMILES: Clc1ccc2oc3ccccc3c2c1-c1ccc(-c2ccccc2)cc1] IM-A1 | 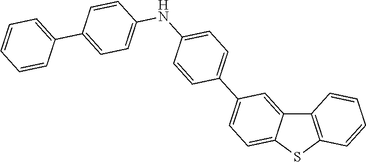 [SMILES: c1ccc(-c2ccc(Nc3ccc(-c4ccc5sc6ccccc6c5c4)cc3)cc2)cc1] IM-B15 | 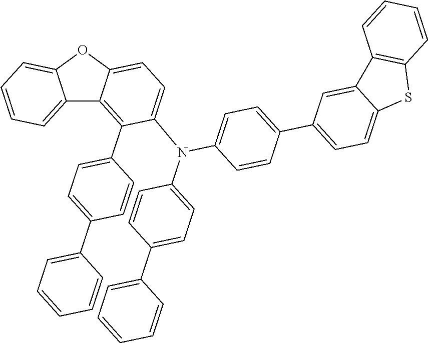 [SMILES: c1ccc(-c2ccc(-c3c(N(c4ccc(-c5ccccc5)cc4)c4ccc(-c5ccc6sc7ccccc7c6c5)cc4)ccc4oc5ccccc5c34)cc2)cc1] 78 | 43.9 | 746.2 |
| 16 | 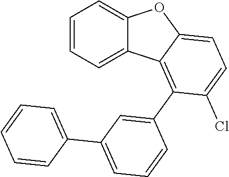 [SMILES: Clc1ccc2oc3ccccc3c2c1-c1cccc(-c2ccccc2)c1] IM-A3 | 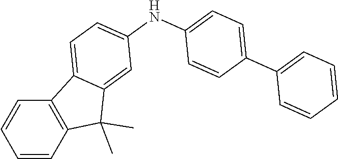 [SMILES: CC1(C)c2ccccc2-c2ccc(Nc3ccc(-c4ccccc4)cc3)cc21] IM-B2 | 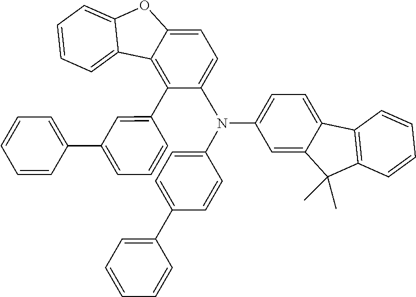 [SMILES: CC1(C)c2ccccc2-c2ccc(N(c3ccc(-c4ccccc4)cc3)c3ccc4oc5ccccc5c4c3-c3cccc(-c4ccccc4)c3)cc21] 99 | 49.3 | 680.3 |
| 17 | 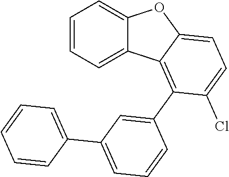 [SMILES: Clc1ccc2oc3ccccc3c2c1-c1cccc(-c2ccccc2)c1] IM-A3 | 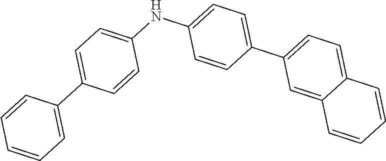 [SMILES: c1ccc(-c2ccc(Nc3ccc(-c4ccc5ccccc5c4)cc3)cc2)cc1] IM-B16 | 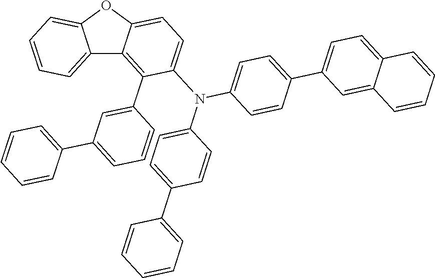 [SMILES: c1ccc(-c2ccc(N(c3ccc(-c4ccc5ccccc5c4)cc3)c3ccc4oc5ccccc5c4c3-c3cccc(-c4ccccc4)c3)cc2)cc1] 116 | 48.8 | 690.3 |
| 18 | 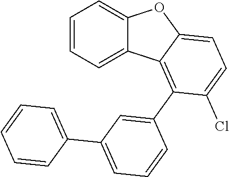 [SMILES: Clc1ccc2oc3ccccc3c2c1-c1cccc(-c2ccccc2)c1] IM-A3 | 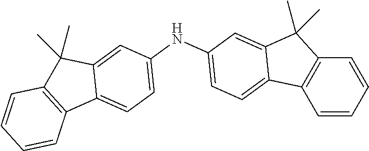 [SMILES: CC1(C)c2ccccc2-c2ccc(Nc3ccc4c(c3)C(C)(C)c3ccccc3-4)cc21] IM-B7 | 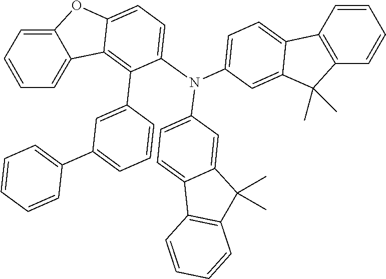 [SMILES: CC1(C)c2ccccc2-c2ccc(N(c3ccc4c(c3)C(C)(C)c3ccccc3-4)c3ccc4oc5ccccc5c4c3-c3cccc(-c4ccccc4)c3)cc21] 121 | 37.2 | 720.3 |
| 19 | 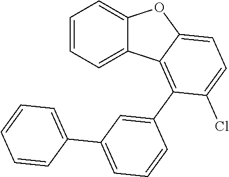 [SMILES: Clc1ccc2oc3ccccc3c2c1-c1cccc(-c2ccccc2)c1] IM-A3 | 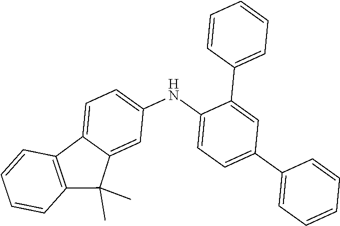 [SMILES: CC1(C)c2ccccc2-c2ccc(Nc3ccc(-c4ccccc4)cc3-c3ccccc3)cc21] IM-B17 | 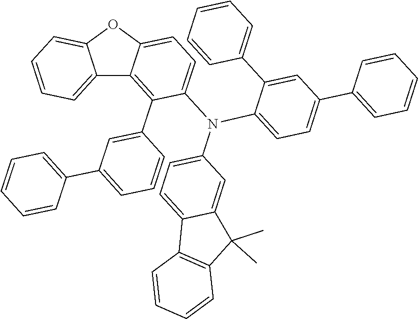 [SMILES: CC1(C)c2ccccc2-c2ccc(N(c3ccc(-c4ccccc4)cc3-c3ccccc3)c3ccc4oc5ccccc5c4c3-c3cccc(-c4ccccc4)c3)cc21] 129 | 37.7 | 756.3 |
| 20 | 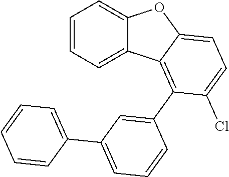 [SMILES: Clc1ccc2oc3ccccc3c2c1-c1cccc(-c2ccccc2)c1] IM-A3 | 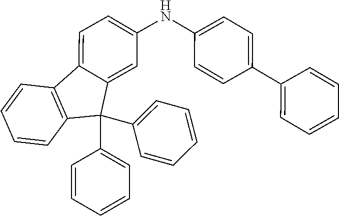 [SMILES: c1ccc(-c2ccc(Nc3ccc4c(c3)C(c3ccccc3)(c3ccccc3)c3ccccc3-4)cc2)cc1] IM-B12 | 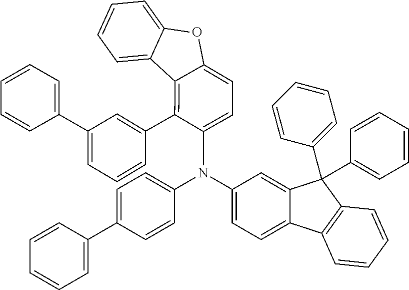 [SMILES: c1ccc(-c2ccc(N(c3ccc4c(c3)C(c3ccccc3)(c3ccccc3)c3ccccc3-4)c3ccc4oc5ccccc5c4c3-c3cccc(-c4ccccc4)c3)cc2)cc1] 148 | 45.6 | 804.3 |
| 21 | 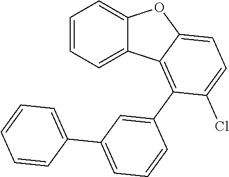 [SMILES: Clc1ccc2oc3ccccc3c2c1-c1cccc(-c2ccccc2)c1] IM-A3 | 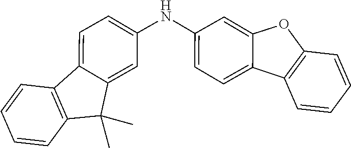 [SMILES: CC1(C)c2ccccc2-c2ccc(Nc3ccc4c(c3)oc3ccccc34)cc21] IM-B13 | 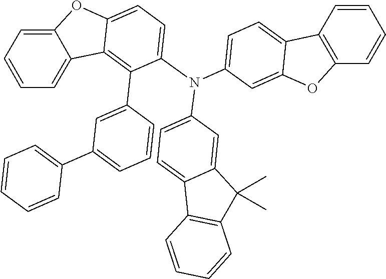 [SMILES: CC1(C)c2ccccc2-c2ccc(N(c3ccc4c(c3)oc3ccccc34)c3ccc4oc5ccccc5c4c3-c3cccc(-c4ccccc4)c3)cc21] 154 | 49.5 | 694.3 |
| 22 | 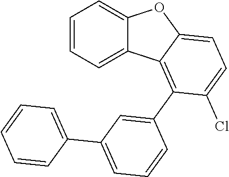 [SMILES: Clc1ccc2oc3ccccc3c2c1-c1cccc(-c2ccccc2)c1] IM-A3 | 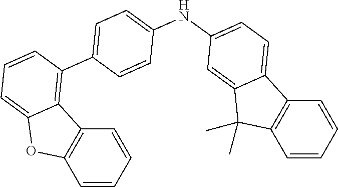 [SMILES: CC1(C)c2ccccc2-c2ccc(Nc3ccc(-c4cccc5oc6ccccc6c45)cc3)cc21] IM-B18 | 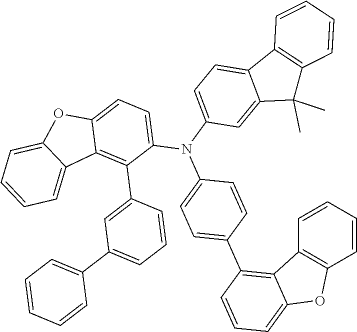 [SMILES: CC1(C)c2ccccc2-c2ccc(N(c3ccc(-c4cccc5oc6ccccc6c45)cc3)c3ccc4oc5ccccc5c4c3-c3cccc(-c4ccccc4)c3)cc21] 171 | 37.2 | 770.3 |
| 23 | 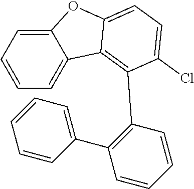 [SMILES: Clc1ccc2oc3ccccc3c2c1-c1ccccc1-c1ccccc1] IM-A4 | 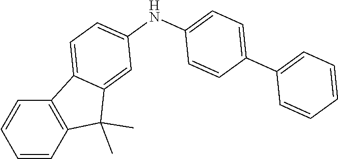 [SMILES: CC1(C)c2ccccc2-c2ccc(Nc3ccc(-c4ccccc4)cc3)cc21] IM-B2 | 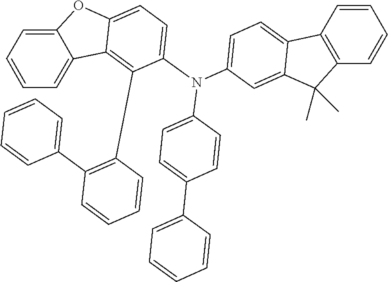 [SMILES: CC1(C)c2ccccc2-c2ccc(N(c3ccc(-c4ccccc4)cc3)c3ccc4oc5ccccc5c4c3-c3ccccc3-c3ccccc3)cc21] 184 | 40.0 | 680.3 |
| 24 | 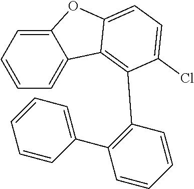 [SMILES: Clc1ccc2oc3ccccc3c2c1-c1ccccc1-c1ccccc1] IM-A4 | 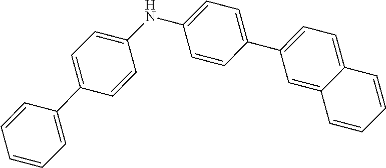 [SMILES: c1ccc(-c2ccc(Nc3ccc(-c4ccc5ccccc5c4)cc3)cc2)cc1] IM-B16 | 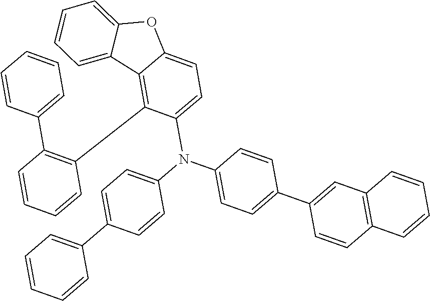 [SMILES: c1ccc(-c2ccc(N(c3ccc(-c4ccc5ccccc5c4)cc3)c3ccc4oc5ccccc5c4c3-c3ccccc3-c3ccccc3)cc2)cc1] 188 | 41.9 | 690.3 |
| 25 | 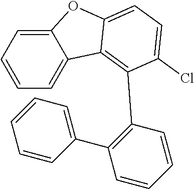 [SMILES: Clc1ccc2oc3ccccc3c2c1-c1ccccc1-c1ccccc1] IM-A4 | 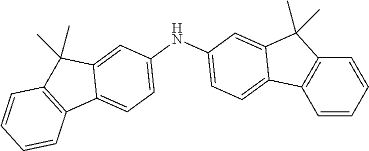 [SMILES: CC1(C)c2ccccc2-c2ccc(Nc3ccc4c(c3)C(C)(C)c3ccccc3-4)cc21] IM-B7 | 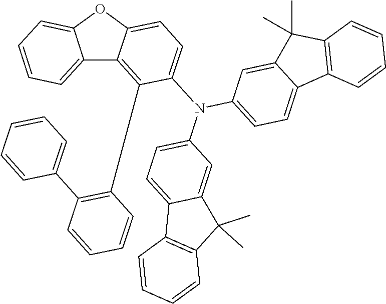 [SMILES: CC1(C)c2ccccc2-c2ccc(N(c3ccc4c(c3)C(C)(C)c3ccccc3-4)c3ccc4oc5ccccc5c4c3-c3ccccc3-c3ccccc3)cc21] 191 | 39.5 | 720.3 |
| 26 | 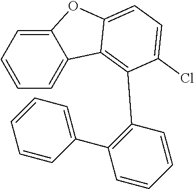 [SMILES: Clc1ccc2oc3ccccc3c2c1-c1ccccc1-c1ccccc1] IM-A4 | 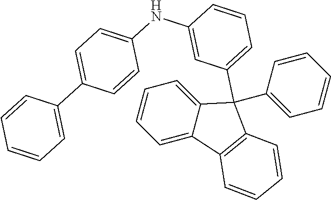 [SMILES: c1ccc(-c2ccc(Nc3cccc(C4(c5ccccc5)c5ccccc5-c5ccccc54)c3)cc2)cc1] IM-B19 | 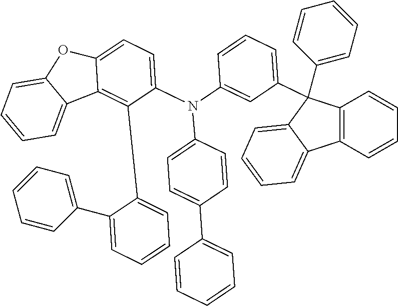 [SMILES: c1ccc(-c2ccc(N(c3cccc(C4(c5ccccc5)c5ccccc5-c5ccccc54)c3)c3ccc4oc5ccccc5c4c3-c3ccccc3-c3ccccc3)cc2)cc1] 196 | 40.8 | 804.3 |
| 27 | 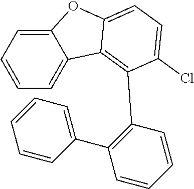 [SMILES: Clc1ccc2oc3ccccc3c2c1-c1ccccc1-c1ccccc1] IM-A4 | 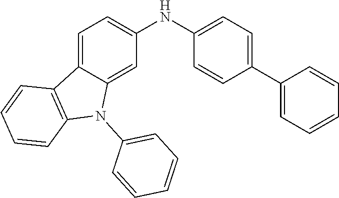 [SMILES: c1ccc(-c2ccc(Nc3ccc4c5ccccc5n(-c5ccccc5)c4c3)cc2)cc1] IM-B20 | 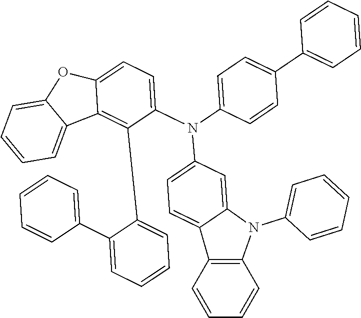 [SMILES: c1ccc(-c2ccc(N(c3ccc4c5ccccc5n(-c5ccccc5)c4c3)c3ccc4oc5ccccc5c4c3-c3ccccc3-c3ccccc3)cc2)cc1] 207 | 40.1 | 729.3 |
| 28 | 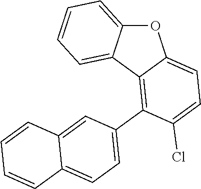 [SMILES: Clc1ccc2oc3ccccc3c2c1-c1ccc2ccccc2c1] IM-A5 | 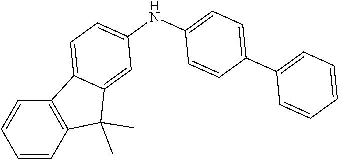 [SMILES: CC1(C)c2ccccc2-c2ccc(Nc3ccc(-c4ccccc4)cc3)cc21] IM-B2 | 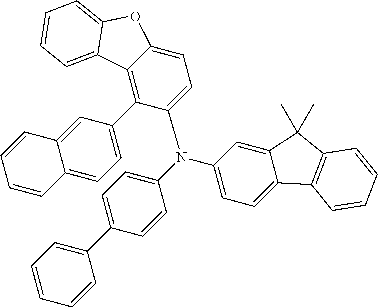 [SMILES: CC1(C)c2ccccc2-c2ccc(N(c3ccc(-c4ccccc4)cc3)c3ccc4oc5ccccc5c4c3-c3ccc4ccccc4c3)cc21] 229 | 43.8 | 654.3 |
| 29 | 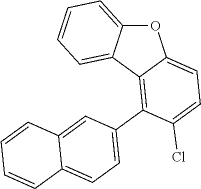 [SMILES: Clc1ccc2oc3ccccc3c2c1-c1ccc2ccccc2c1] IM-A5 | 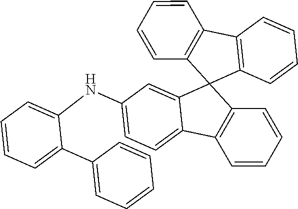 [SMILES: c1ccc(-c2ccccc2Nc2ccc3c(c2)C2(c4ccccc4-c4ccccc42)c2ccccc2-3)cc1] IM-B3 | 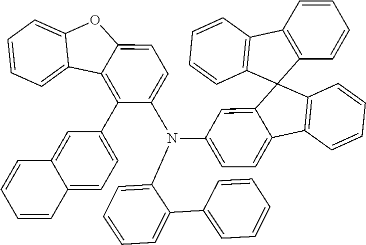 [SMILES: c1ccc(-c2ccccc2N(c2ccc3c(c2)C2(c4ccccc4-c4ccccc42)c2ccccc2-3)c2ccc3oc4ccccc4c3c2-c2ccc3ccccc3c2)cc1] 235 | 40.0 | 776.3 |
| 30 | 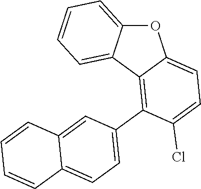 [SMILES: Clc1ccc2oc3ccccc3c2c1-c1ccc2ccccc2c1] IM-A5 | 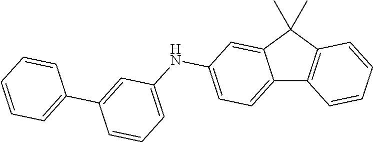 [SMILES: CC1(C)c2ccccc2-c2ccc(Nc3cccc(-c4ccccc4)c3)cc21] IM-B4 | 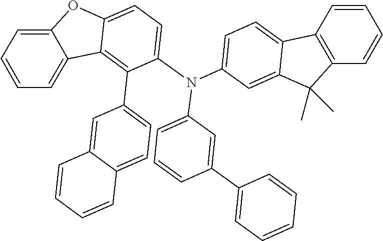 [SMILES: CC1(C)c2ccccc2-c2ccc(N(c3cccc(-c4ccccc4)c3)c3ccc4oc5ccccc5c4c3-c3ccc4ccccc4c3)cc21] 239 | 41.8 | 654.3 |
| 31 | 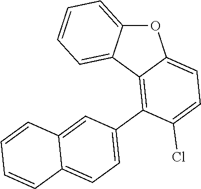 [SMILES: Clc1ccc2oc3ccccc3c2c1-c1ccc2ccccc2c1] IM-A5 | 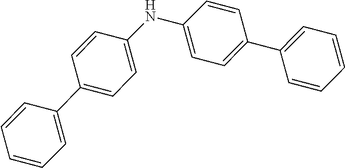 [SMILES: c1ccc(-c2ccc(Nc3ccc(-c4ccccc4)cc3)cc2)cc1] IM-B5 | 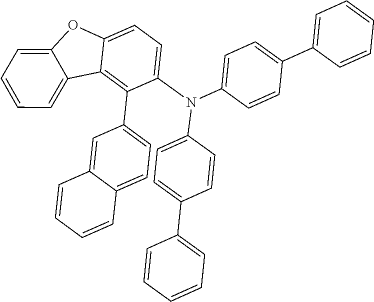 [SMILES: c1ccc(-c2ccc(N(c3ccc(-c4ccccc4)cc3)c3ccc4oc5ccccc5c4c3-c3ccc4ccccc4c3)cc2)cc1] 241 | 45.9 | 614.2 |
| 32 | 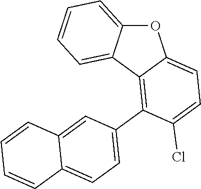 [SMILES: Clc1ccc2oc3ccccc3c2c1-c1ccc2ccccc2c1] IM-A5 | 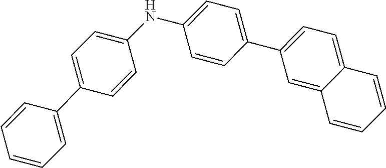 [SMILES: c1ccc(-c2ccc(Nc3ccc(-c4ccc5ccccc5c4)cc3)cc2)cc1] IM-B16 | 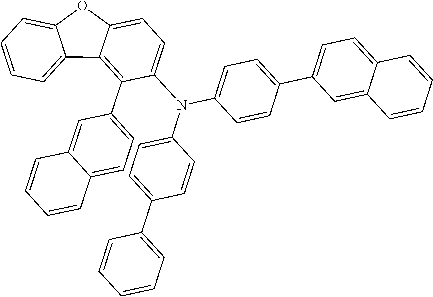 [SMILES: c1ccc(-c2ccc(N(c3ccc(-c4ccc5ccccc5c4)cc3)c3ccc4oc5ccccc5c4c3-c3ccc4ccccc4c3)cc2)cc1] 246 | 42.4 | 664.2 |
| 33 | 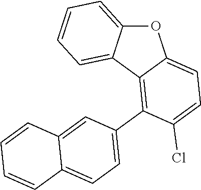 [SMILES: Clc1ccc2oc3ccccc3c2c1-c1ccc2ccccc2c1] IM-A5 | 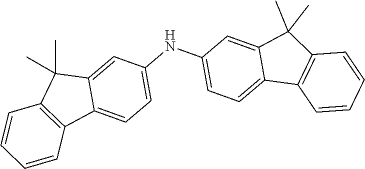 [SMILES: CC1(C)c2ccccc2-c2ccc(Nc3ccc4c(c3)C(C)(C)c3ccccc3-4)cc21] IM-B7 | 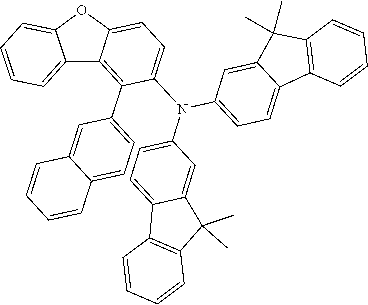 [SMILES: CC1(C)c2ccccc2-c2ccc(N(c3ccc4c(c3)C(C)(C)c3ccccc3-4)c3ccc4oc5ccccc5c4c3-c3ccc4ccccc4c3)cc21] 251 | 47.1 | 694.3 |
| 34 | 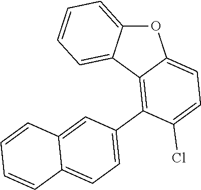 [SMILES: Clc1ccc2oc3ccccc3c2c1-c1ccc2ccccc2c1] IM-A5 | 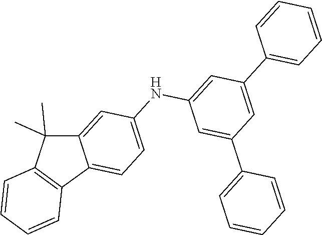 [SMILES: CC1(C)c2ccccc2-c2ccc(Nc3cc(-c4ccccc4)cc(-c4ccccc4)c3)cc21] IM-B9 | 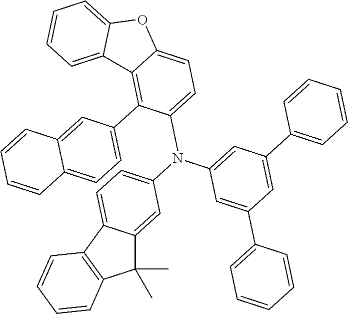 [SMILES: CC1(C)c2ccccc2-c2ccc(N(c3cc(-c4ccccc4)cc(-c4ccccc4)c3)c3ccc4oc5ccccc5c4c3-c3ccc4ccccc4c3)cc21] 260 | 40.2 | 730.3 |
| 35 | 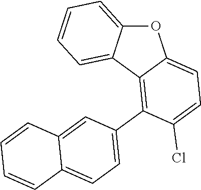 [SMILES: Clc1ccc2oc3ccccc3c2c1-c1ccc2ccccc2c1] IM-A5 | 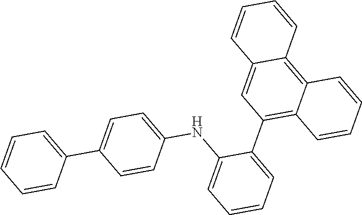 [SMILES: c1ccc(-c2ccc(Nc3ccccc3-c3cc4ccccc4c4ccccc34)cc2)cc1] IM-B21 | 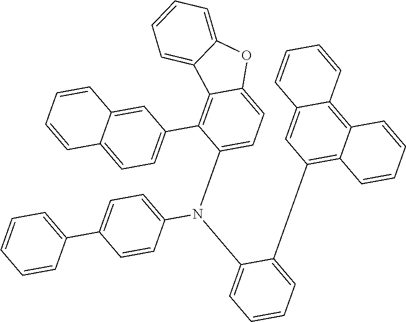 [SMILES: c1ccc(-c2ccc(N(Cc3ccccc3CCc3cc4ccccc4c4ccccc34)Cc3ccc4oc5ccccc5c4c3-c3ccc4ccccc4c3)cc2)cc1] 268 | 36.6 | 714.3 |
| 36 | 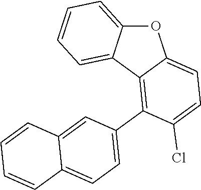 [SMILES: Clc1ccc2oc3ccccc3c2c1-c1ccc2ccccc2c1] IM-A5 | 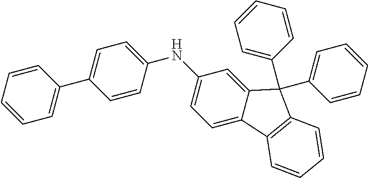 [SMILES: c1ccc(-c2ccc(Nc3ccc4c(c3)C(c3ccccc3)(c3ccccc3)c3ccccc3-4)cc2)cc1] IM-B12 | 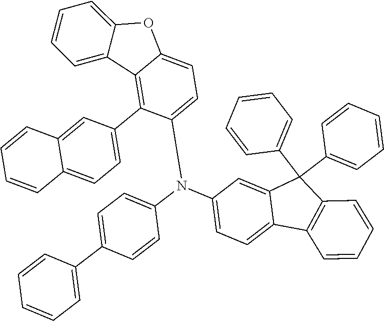 [SMILES: c1ccc(-c2ccc(N(Cc3ccc4oc5ccccc5c4c3-c3ccc4ccccc4c3)c3ccc4c(c3)C(c3ccccc3)(c3ccccc3)c3ccccc3-4)cc2)cc1] 938 | 48.9 | 778.3 |
| 37 | 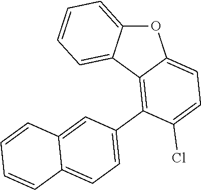 [SMILES: Clc1ccc2oc3ccccc3c2c1-c1ccc2ccccc2c1] IM-A5 | 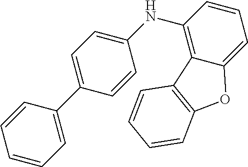 [SMILES: c1ccc(-c2ccc(Nc3cccc4oc5ccccc5c34)cc2)cc1] IM-B22 | 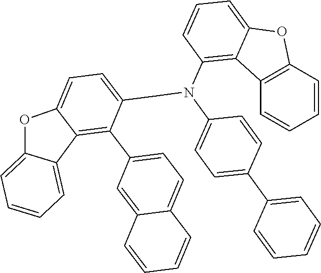 [SMILES: c1ccc(-c2ccc(N(Cc3ccc4oc5ccccc5c4c3-c3ccc4ccccc4c3)c3cccc4oc5ccccc5c34)cc2)cc1] 943 | 41.0 | 628.2 |
| 38 | 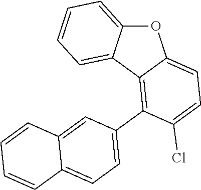 [SMILES: Clc1ccc2oc3ccccc3c2c1-c1ccc2ccccc2c1] IM-A5 | 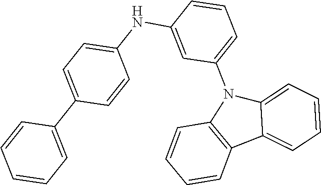 [SMILES: c1ccc(-c2ccc(Nc3cccc(-n4c5ccccc5c5ccccc54)c3)cc2)cc1] IM-B23 | 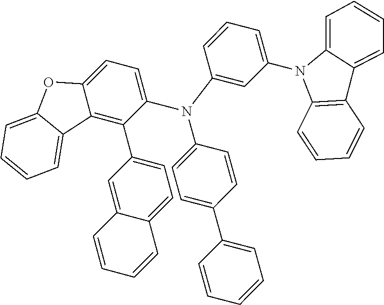 [SMILES: c1ccc(-c2ccc(N(c3cccc(-n4c5ccccc5c5ccccc54)c3)c3ccc4oc5ccccc5c4c3-c3ccc4ccccc4c3)cc2)cc1] 950 | 46.1 | 703.3 |
| 39 | 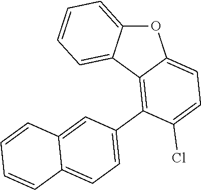 [SMILES: Clc1ccc2oc3ccccc3c2c1-c1ccc2ccccc2c1] IM-A5 | 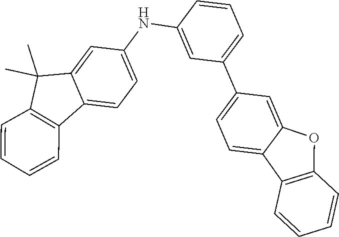 [SMILES: CC1(C)c2ccccc2-c2ccc(Nc3cccc(-c4ccc5c(c4)oc4ccccc45)c3)cc21] IM-B24 | 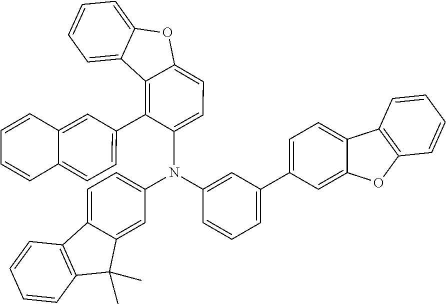 [SMILES: CC1(C)c2ccccc2-c2ccc(N(c3cccc(-c4ccc5c(c4)oc4ccccc45)c3)c3ccc4oc5ccccc5c4c3-c3ccc4ccccc4c3)cc21] 273 | 46.6 | 744.3 |
| 40 | 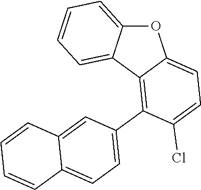 [SMILES: Clc1ccc2oc3ccccc3c2c1-c1ccc2ccccc2c1] IM-A5 | 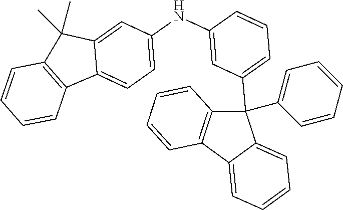 [SMILES: CC1(C)c2ccccc2-c2ccc(Nc3cccc(C4(c5ccccc5)c5ccccc5-c5ccccc54)c3)cc21] IM-B25 | 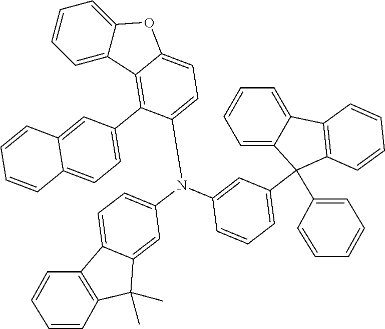 [SMILES: CC1(C)c2ccccc2-c2ccc(N(Cc3ccc4oc5ccccc5c4c3-c3ccc4ccccc4c3)c3cccc(C4(c5ccccc5)c5ccccc5-c5ccccc54)c3)cc21] 281 | 46.8 | 818.3 |
| 41 | 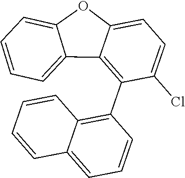 [SMILES: Clc1ccc2oc3ccccc3c2c1-c1cccc2ccccc12] IM-A6 | 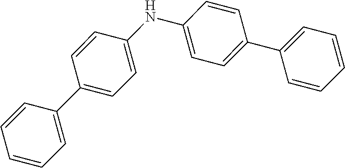 [SMILES: c1ccc(-c2ccc(Nc3ccc(-c4ccccc4)cc3)cc2)cc1] IM-B5 | 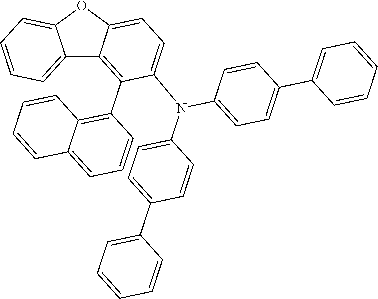 [SMILES: c1ccc(-c2ccc(N(c3ccc(-c4ccccc4)cc3)c3ccc4oc5ccccc5c4c3-c3cccc4ccccc34)cc2)cc1] 291 | 47.8 | 614.2 |
| 42 | 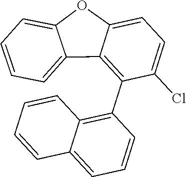 [SMILES: Clc1ccc2oc3ccccc3c2c1-c1cccc2ccccc12] IM-A6 | 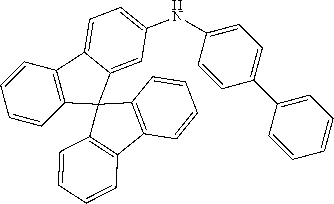 [SMILES: c1ccc(-c2ccc(Nc3ccc4c(c3)C3(c5ccccc5-c5ccccc53)c3ccccc3-4)cc2)cc1] IM-B32 | 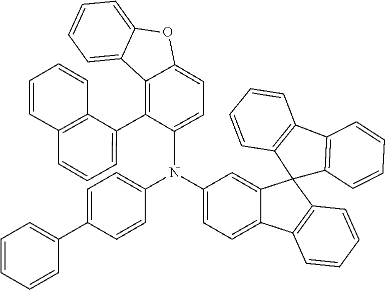 [SMILES: c1ccc(-c2ccc(N(c3ccc4c(c3)C3(c5ccccc5-c5ccccc53)c3ccccc3-4)c3ccc4oc5ccccc5c4c3-c3cccc4ccccc34)cc2)cc1] 290 | 43.4 | 776.3 |
| 43 | 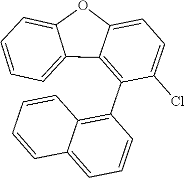 [SMILES: Clc1ccc2oc3ccccc3c2c1-c1cccc2ccccc12] IM-A6 | 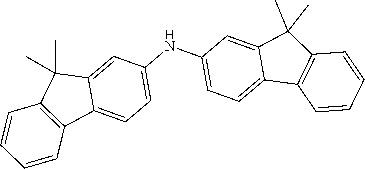 [SMILES: CC1(C)c2ccccc2-c2ccc(Nc3ccc4c(c3)C(C)(C)c3ccccc3-4)cc21] IM-B7 | 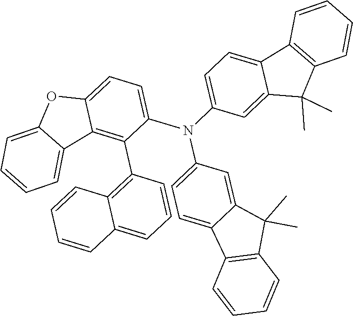 [SMILES: CC1(C)c2ccccc2-c2ccc(N(c3ccc4c(c3)C(C)(C)c3ccccc3-4)c3ccc4oc5ccccc5c4c3-c3cccc4ccccc34)cc21] 296 | 48.5 | 694.3 |
| 44 | 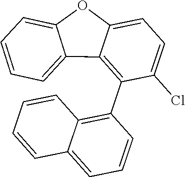 [SMILES: Clc1ccc2oc3ccccc3c2c1-c1cccc2ccccc12] IM-A6 | 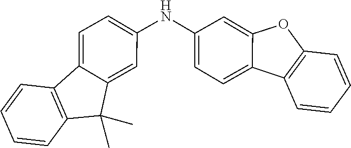 [SMILES: CC1(C)c2ccccc2-c2ccc(Nc3ccc4c(c3)oc3ccccc34)cc21] IM-B13 | 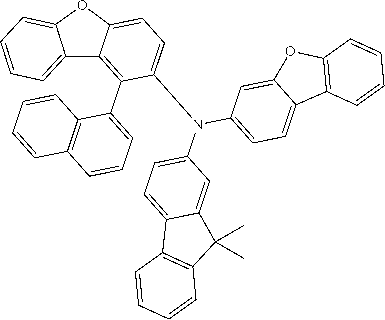 [SMILES: CC1(C)c2ccccc2-c2ccc(N(Cc3ccc4oc5ccccc5c4c3-c3cccc4ccccc34)c3ccc4c(c3)oc3ccccc34)cc21] 309 | 40.2 | 668.2 |
| 45 | 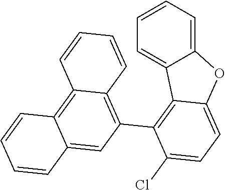 [SMILES: Clc1ccc2oc3ccccc3c2c1-c1cc2ccccc2c2ccccc12] IM-A7 | 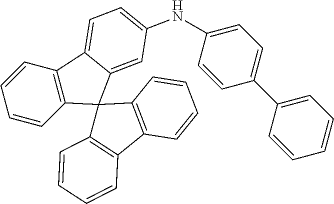 [SMILES: c1ccc(-c2ccc(Nc3ccc4c(c3)C3(c5ccccc5-c5ccccc53)c3ccccc3-4)cc2)cc1] IM-B32 | 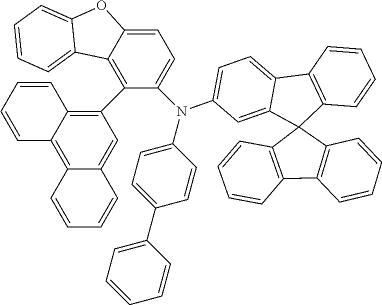 [SMILES: c1ccc(-c2ccc(N(c3ccc4c(c3)C3(c5ccccc5-c5ccccc53)c3ccccc3-4)c3ccc4oc5ccccc5c4c3-c3cc4ccccc4c4ccccc34)cc2)cc1] 340 | 37.6 | 626.3 |
| 46 | 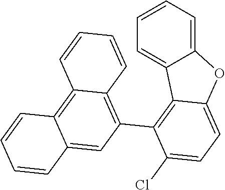 [SMILES: Clc1ccc2oc3ccccc3c2c1-c1cc2ccccc2c2ccccc12] IM-A7 | 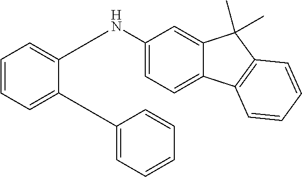 [SMILES: CC1(C)c2ccccc2-c2ccc(NCc3ccccc3Cc3ccccc3)cc21] IM-B26 | 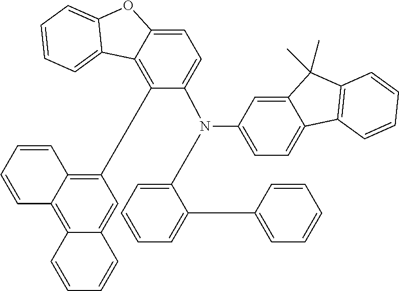 [SMILES: CC1(C)c2ccccc2-c2ccc(N(Cc3ccccc3Cc3ccccc3)c3ccc4oc5ccccc5c4c3Cc3cc4ccccc4c4ccccc34)cc21] 344 | 42.1 | 704.3 |
| 47 | 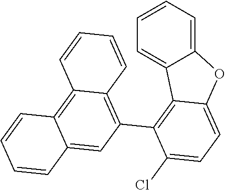 [SMILES: Clc1ccc2oc3ccccc3c2c1-c1cc2ccccc2c2ccccc12] IM-A7 | 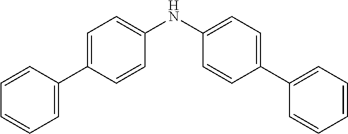 [SMILES: c1ccc(-c2ccc(Nc3ccc(-c4ccccc4)cc3)cc2)cc1] IM-B5 | 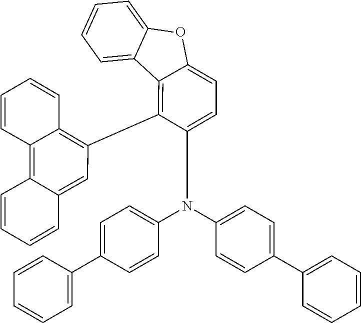 [SMILES: c1ccc(-c2ccc(N(c3ccc(-c4ccccc4)cc3)c3ccc4oc5ccccc5c4c3-c3cc4ccccc4c4ccccc34)cc2)cc1] 351 | 40.9 | 664.2 |
| 48 | 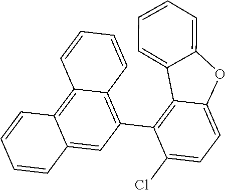 [SMILES: Clc1ccc2oc3ccccc3c2c1-c1cc2ccccc2c2ccccc12] IM-A7 | 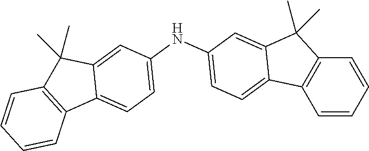 [SMILES: CC1(C)c2ccccc2-c2ccc(Nc3ccc4c(c3)C(C)(C)c3ccccc3-4)cc21] IM-B7 | 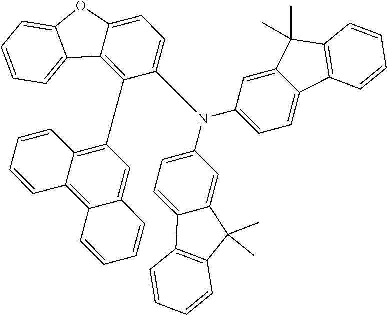 [SMILES: CC1(C)c2ccccc2-c2ccc(N(Cc3ccc4oc5ccccc5c4c3Cc3cc4ccccc4c4ccccc34)c3ccc4c(c3)C(C)(C)c3ccccc3-4)cc21] 361 | 50.5 | 744.3 |
| 49 | 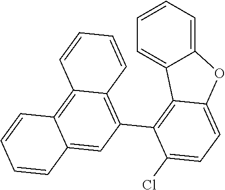 [SMILES: Clc1ccc2oc3ccccc3c2c1-c1cc2ccccc2c2ccccc12] IM-A7 | 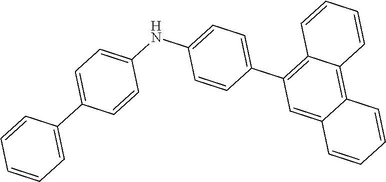 [SMILES: c1ccc(-c2ccc(Nc3ccc(-c4cc5ccccc5c5ccccc45)cc3)cc2)cc1] IM-B10 | 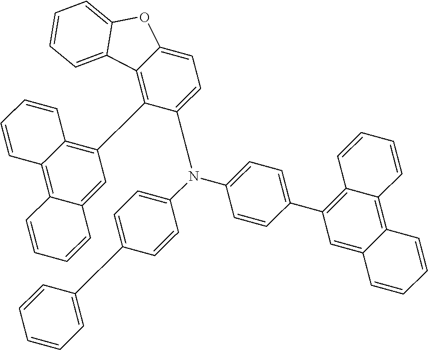 [SMILES: c1ccc(Cc2ccc(N(Cc3ccc4oc5ccccc5c4c3Cc3cc4ccccc4c4ccccc34)c3ccc(-c4cc5ccccc5c5ccccc45)cc3)cc2)cc1] 376 | 50.2 | 764.3 |
| 50 | 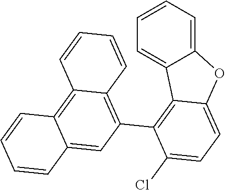 [SMILES: Clc1ccc2oc3ccccc3c2c1-c1cc2ccccc2c2ccccc12] IM-A7 | 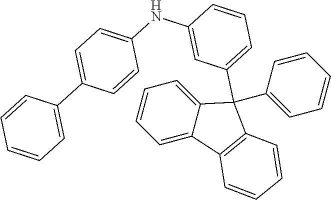 [SMILES: c1ccc(-c2ccc(Nc3cccc(C4(c5ccccc5)c5ccccc5-c5ccccc54)c3)cc2)cc1] IM-B19 | 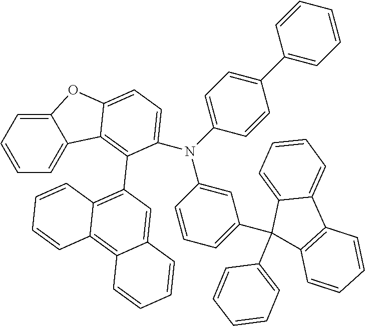 [SMILES: c1ccc(-c2ccc(N(c3cccc(C4(c5ccccc5)c5ccccc5-c5ccccc54)c3)c3ccc4oc5ccccc5c4c3-c3cc4ccccc4c4ccccc34)cc2)cc1] 386 | 48.9 | 828.3 |
| 51 | 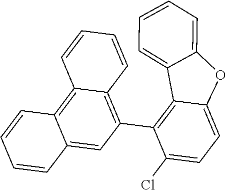 [SMILES: Clc1ccc2oc3ccccc3c2c1-c1cc2ccccc2c2ccccc12] IM-A7 | 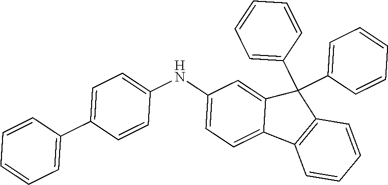 [SMILES: c1ccc(-c2ccc(Nc3ccc4c(c3)C(c3ccccc3)(c3ccccc3)c3ccccc3-4)cc2)cc1] IM-B12 | 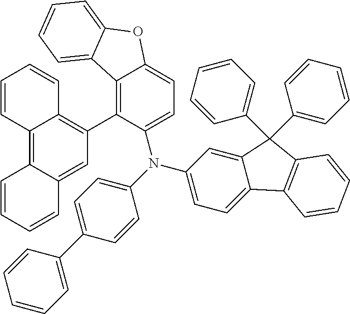 [SMILES: c1ccc(-c2ccc(N(c3ccc4c(c3)C(c3ccccc3)(c3ccccc3)c3ccccc3-4)c3ccc4oc5ccccc5c4c3-c3cc4ccccc4c4ccccc34)cc2)cc1] 388 | 37.9 | 828.3 |
| 52 | 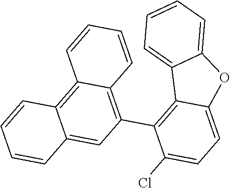 [SMILES: Clc1ccc2oc3ccccc3c2c1-c1cc2ccccc2c2ccccc12] IM-A7 | 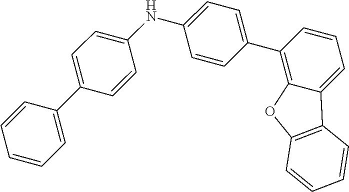 [SMILES: c1ccc(-c2ccc(Nc3ccc(-c4cccc5c4oc4ccccc45)cc3)cc2)cc1] IM-B27 | 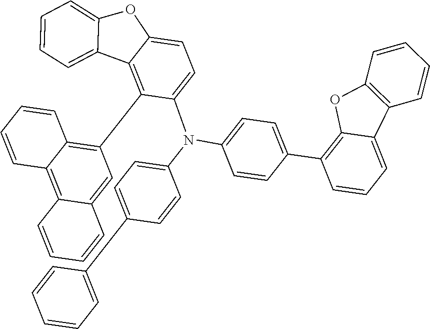 [SMILES: c1ccc(Cc2ccc(N(c3ccc(-c4cccc5c4oc4ccccc45)cc3)c3ccc4oc5ccccc5c4c3Cc3cc4ccccc4c4ccccc34)cc2)cc1] 402 | 39.3 | 754.3 |
| 53 | 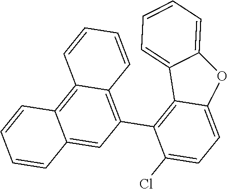 [SMILES: Clc1ccc2oc3ccccc3c2c1-c1cc2ccccc2c2ccccc12] IM-A7 | 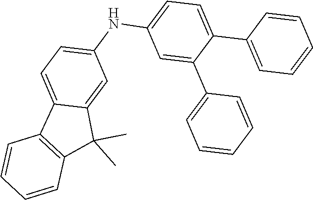 [SMILES: CC1(C)c2ccccc2-c2ccc(Nc3ccc(-c4ccccc4)c(-c4ccccc4)c3)cc21] IM-B28 | 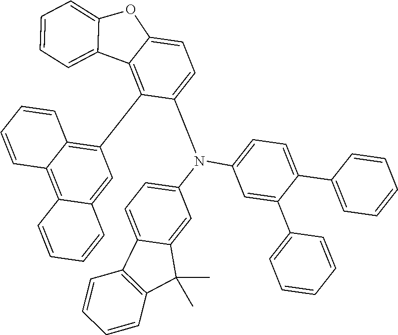 [SMILES: CC1(C)c2ccccc2-c2ccc(N(Cc3ccc4oc5ccccc5c4c3Cc3cc4ccccc4c4ccccc34)c3ccc(-c4ccccc4)c(-c4ccccc4)c3)cc21] 419 | 41.6 | 780.3 |
| 54 | 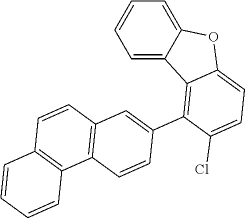 [SMILES: Clc1ccc2oc3ccccc3c2c1-c1ccc2c(ccc3ccccc32)c1] IM-A8 | 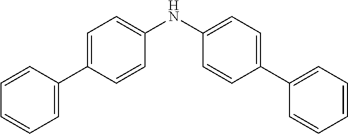 [SMILES: c1ccc(-c2ccc(Nc3ccc(-c4ccccc4)cc3)cc2)cc1] IM-B5 | 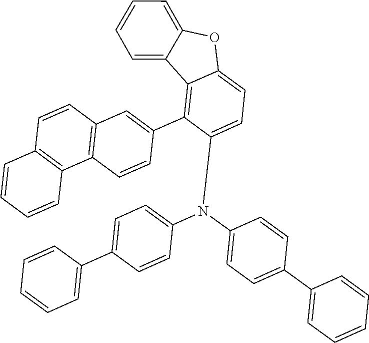 [SMILES: c1ccc(-c2ccc(N(c3ccc(-c4ccccc4)cc3)c3ccc4oc5ccccc5c4c3-c3ccc4c(ccc5ccccc54)c3)cc2)cc1] 422 | 48.6 | 664.2 |
| 55 | 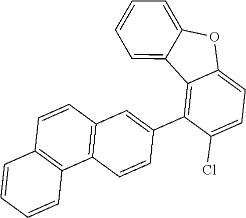 [SMILES: Clc1ccc2oc3ccccc3c2c1-c1ccc2c(ccc3ccccc32)c1] IM-A8 | 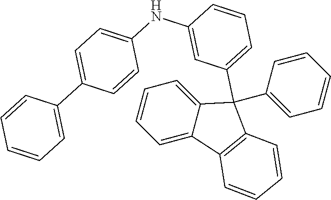 [SMILES: c1ccc(-c2ccc(Nc3cccc(C4(c5ccccc5)c5ccccc5-c5ccccc54)c3)cc2)cc1] IM-B19 | 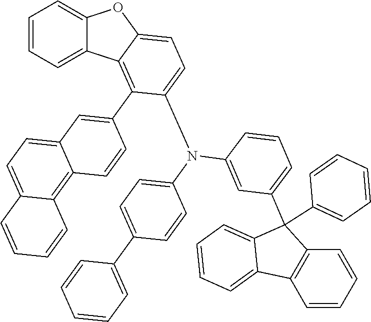 [SMILES: c1ccc(-c2ccc(N(Cc3ccc4oc5ccccc5c4c3-c3ccc4c(ccc5ccccc54)c3)c3cccc(C4(c5ccccc5)c5ccccc5-c5ccccc54)c3)cc2)cc1] 436 | 38.3 | 828.3 |
| 56 | 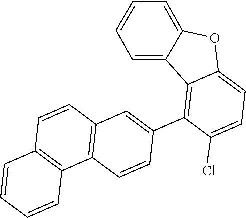 [SMILES: Clc1ccc2oc3ccccc3c2c1-c1ccc2c(ccc3ccccc32)c1] IM-A8 | 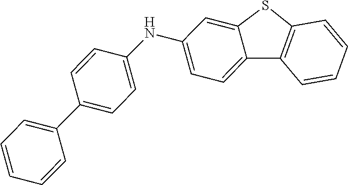 [SMILES: c1ccc(-c2ccc(Nc3ccc4c(c3)sc3ccccc34)cc2)cc1] IM-B29 | 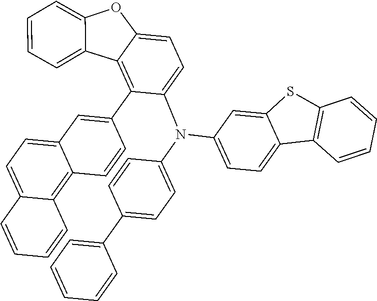 [SMILES: c1ccc(-c2ccc(N(c3ccc4c(c3)sc3ccccc34)c3ccc4oc5ccccc5c4c3-c3ccc4c(ccc5ccccc54)c3)cc2)cc1] 441 | 49.9 | 694.2 |
| 57 | 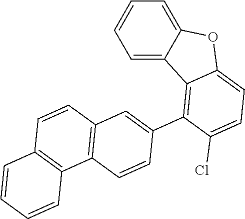 [SMILES: Clc1ccc2oc3ccccc3c2c1-c1ccc2c(ccc3ccccc32)c1] IM-A8 | 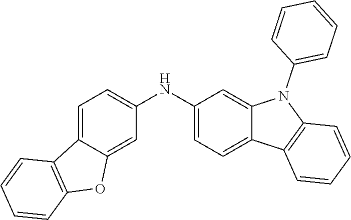 [SMILES: c1ccc(-n2c3ccccc3c3ccc(Nc4ccc5c(c4)oc4ccccc45)cc32)cc1] IM-B30 | 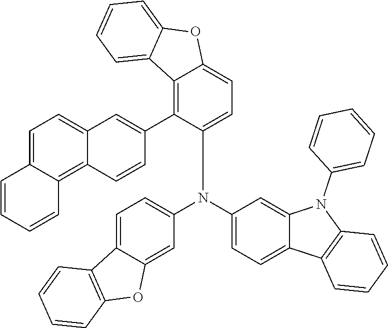 [SMILES: c1ccc(-n2c3ccccc3c3ccc(N(Cc4ccc5oc6ccccc6c5c4-c4ccc5c(ccc6ccccc65)c4)c4ccc5c(c4)oc4ccccc45)cc32)cc1] 450 | 40.5 | 767.3 |
| 58 | 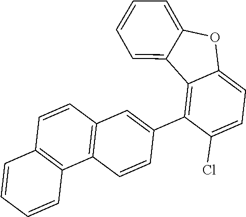 [SMILES: Clc1ccc2oc3ccccc3c2c1-c1ccc2c(ccc3ccccc32)c1] IM-A8 | 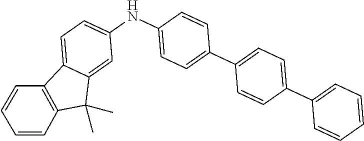 [SMILES: CC1(C)c2ccccc2-c2ccc(Nc3ccc(-c4ccc(-c5ccccc5)cc4)cc3)cc21] IM-B31 | 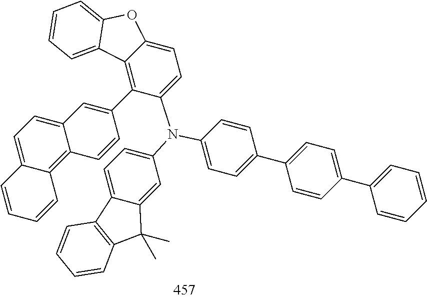 [SMILES: CC1(C)c2ccccc2-c2ccc(N(c3ccc(-c4ccc(-c5ccccc5)cc4)cc3)c3ccc4oc5ccccc5c4c3-c3ccc4c(ccc5ccccc54)c3)cc21] | 39.9 | 780.3 |
| 59 | 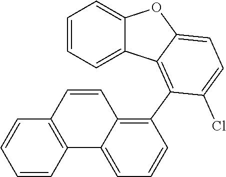 [SMILES: Clc1ccc2oc3ccccc3c2c1-c1cccc2c1ccc1ccccc12] IM-A9 | 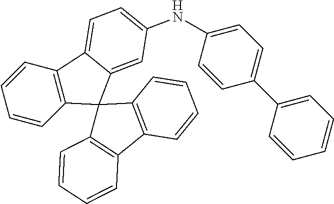 [SMILES: c1ccc(-c2ccc(Nc3ccc4c(c3)C3(c5ccccc5-c5ccccc53)c3ccccc3-4)cc2)cc1] IM-B32 | 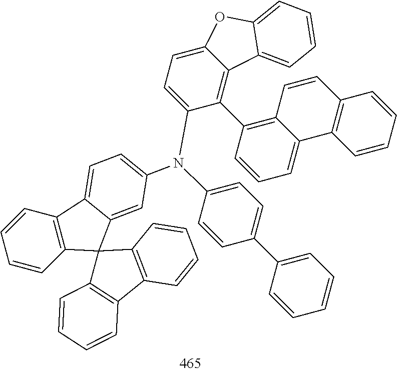 [SMILES: c1ccc(-c2ccc(N(c3ccc4c(c3)C3(c5ccccc5-c5ccccc53)c3ccccc3-4)c3ccc4oc5ccccc5c4c3-c3cccc4c3ccc3ccccc34)cc2)cc1] | 37.3 | 826.3 |
| 60 | 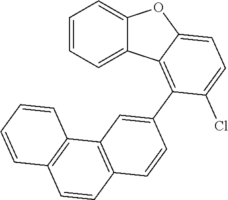 [SMILES: Clc1ccc2oc3ccccc3c2c1-c1ccc2ccc3ccccc3c2c1] IM-A10 | 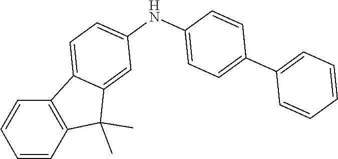 [SMILES: CC1(C)c2ccccc2-c2ccc(Nc3ccc(-c4ccccc4)cc3)cc21] IM-B2 | 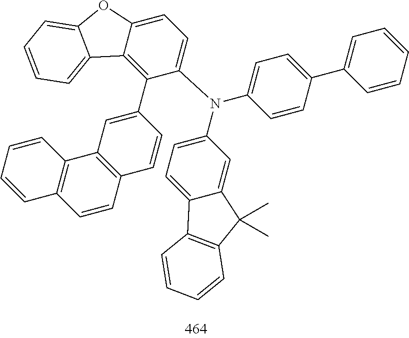 [SMILES: CC1(C)c2ccccc2-c2ccc(N(c3ccc(-c4ccccc4)cc3)c3ccc4oc5ccccc5c4c3-c3ccc4ccc5ccccc5c4c3)cc21] | 40.2 | 704.3 |
| 61 | 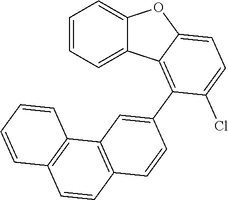 [SMILES: Clc1ccc2oc3ccccc3c2c1-c1ccc2ccc3ccccc3c2c1] IM-A10 | 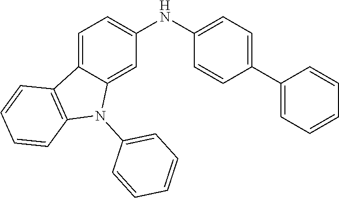 [SMILES: c1ccc(-c2ccc(Nc3ccc4c5ccccc5n(-c5ccccc5)c4c3)cc2)cc1] IM-B20 | 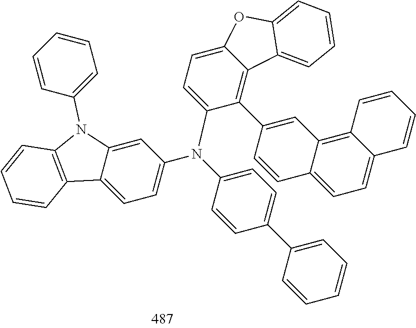 [SMILES: c1ccc(-c2ccc(N(c3ccc4c5ccccc5n(-c5ccccc5)c4c3)c3ccc4oc5ccccc5c4c3-c3ccc4ccc5ccccc5c4c3)cc2)cc1] | 42.5 | 753.3 |
| 62 | 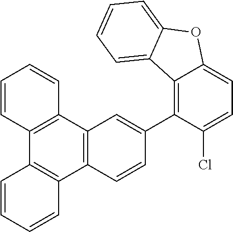 [SMILES: Clc1ccc2oc3ccccc3c2c1-c1ccc2c3ccccc3c3ccccc3c2c1] IM-A11 | 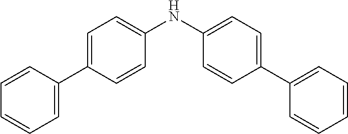 [SMILES: c1ccc(-c2ccc(Nc3ccc(-c4ccccc4)cc3)cc2)cc1] IM-B5 | 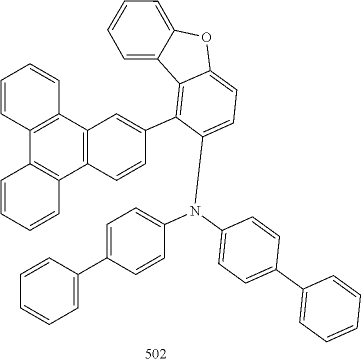 [SMILES: c1ccc(-c2ccc(N(c3ccc(-c4ccccc4)cc3)c3ccc4oc5ccccc5c4c3-c3ccc4c5ccccc5c5ccccc5c4c3)cc2)cc1] | 49.0 | 714.3 |
| 63 | 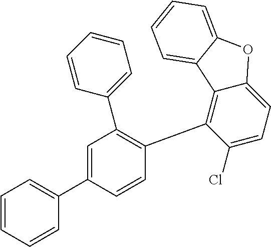 [SMILES: Clc1ccc2oc3ccccc3c2c1-c1ccc(-c2ccccc2)cc1-c1ccccc1] IM-A12 | 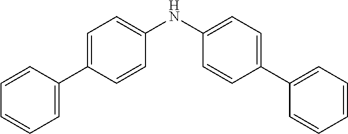 [SMILES: c1ccc(-c2ccc(Nc3ccc(-c4ccccc4)cc3)cc2)cc1] IM-B5 | 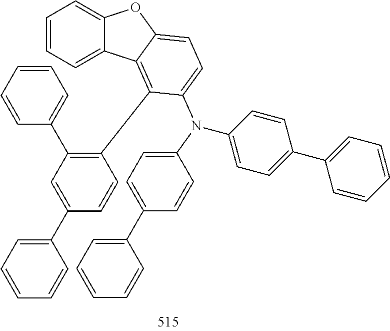 [SMILES: c1ccc(-c2ccc(N(c3ccc(-c4ccccc4)cc3)c3ccc4oc5ccccc5c4c3-c3ccc(-c4ccccc4)cc3-c3ccccc3)cc2)cc1] | 40.0 | 716.3 |
| 64 | 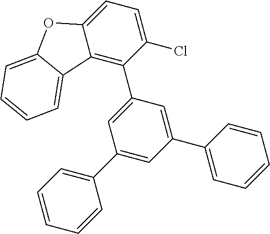 [SMILES: Clc1ccc2oc3ccccc3c2c1-c1cc(-c2ccccc2)cc(-c2ccccc2)c1] IM-A13 | 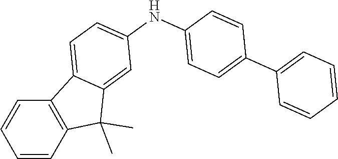 [SMILES: CC1(C)c2ccccc2-c2ccc(Nc3ccc(-c4ccccc4)cc3)cc21] IM-B2 | 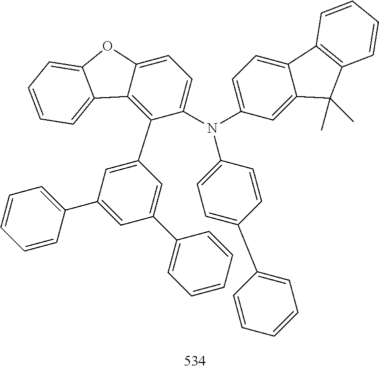 [SMILES: CC1(C)c2ccccc2-c2ccc(N(c3ccc(-c4ccccc4)cc3)c3ccc4oc5ccccc5c4c3-c3cc(-c4ccccc4)cc(-c4ccccc4)c3)cc21] | 41.4 | 756.3 |
| 65 | 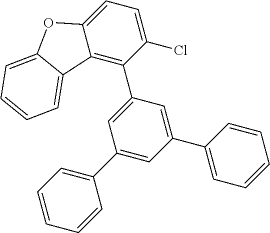 [SMILES: Clc1ccc2oc3ccccc3c2c1-c1cc(-c2ccccc2)cc(-c2ccccc2)c1] IM-A13 | 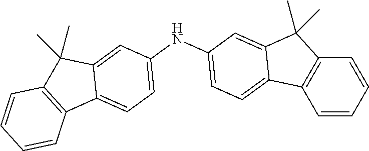 [SMILES: CC1(C)c2ccccc2-c2ccc(Nc3ccc4c(c3)C(C)(C)c3ccccc3-4)cc21] IM-B7 | 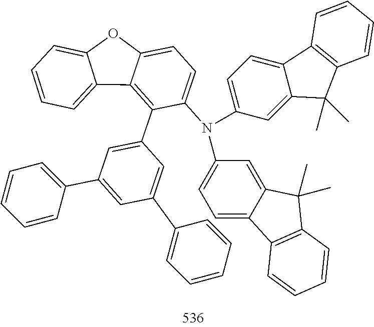 [SMILES: CC1(C)c2ccccc2-c2ccc(N(c3ccc4c(c3)C(C)(C)c3ccccc3-4)c3ccc4oc5ccccc5c4c3-c3cc(-c4ccccc4)cc(-c4ccccc4)c3)cc21] | 43.9 | 796.4 |
| 66 | 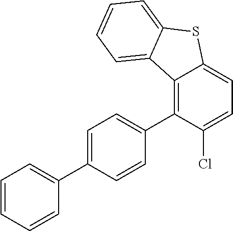 [SMILES: Clc1ccc2sc3ccccc3c2c1-c1ccc(-c2ccccc2)cc1] IM-A14 | 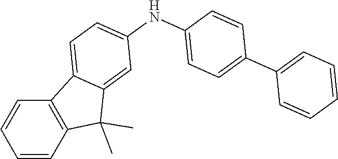 [SMILES: CC1(C)c2ccccc2-c2ccc(Nc3ccc(-c4ccccc4)cc3)cc21] IM-B2 | 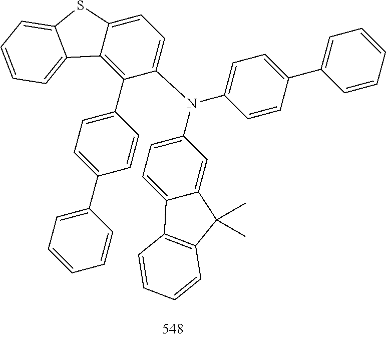 [SMILES: CC1(C)c2ccccc2-c2ccc(N(c3ccc(-c4ccccc4)cc3)c3ccc4sc5ccccc5c4c3-c3ccc(-c4ccccc4)cc3)cc21] | 49.3 | 696.3 |
| 67 | 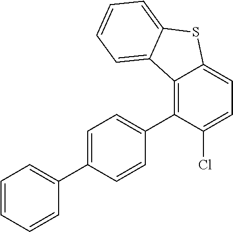 [SMILES: Clc1ccc2sc3ccccc3c2c1-c1ccc(-c2ccccc2)cc1] IM-A14 | 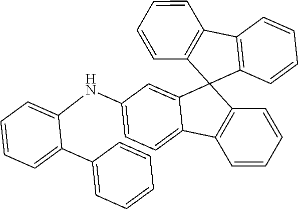 [SMILES: c1ccc(-c2ccccc2Nc2ccc3c(c2)C2(c4ccccc4-c4ccccc42)c2ccccc2-3)cc1] IM-B3 | 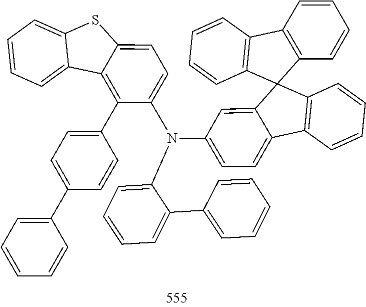 [SMILES: c1ccc(-c2ccc(-c3c(N(c4ccc5c(c4)C4(c6ccccc6-c6ccccc64)c4ccccc4-5)c4ccccc4-c4ccccc4)ccc4sc5ccccc5c34)cc2)cc1] | 48.8 | 818.3 |
| 68 | 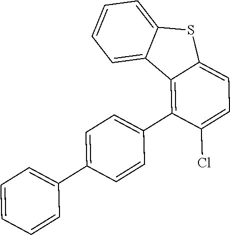 [SMILES: Clc1ccc2sc3ccccc3c2c1-c1ccc(-c2ccccc2)cc1] IM-A14 | 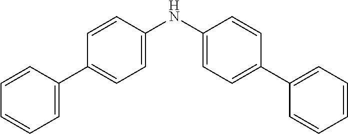 [SMILES: c1ccc(-c2ccc(Nc3ccc(-c4ccccc4)cc3)cc2)cc1] IM-B5 | 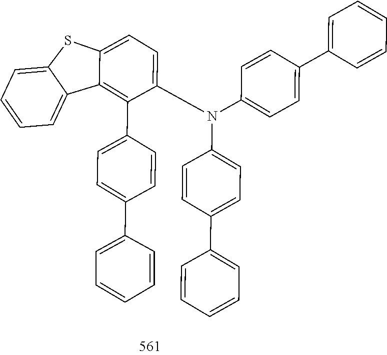 [SMILES: c1ccc(-c2ccc(-c3c(N(c4ccc(-c5ccccc5)cc4)c4ccc(-c5ccccc5)cc4)ccc4sc5ccccc5c34)cc2)cc1] | 37.2 | 656.2 |
| 69 | 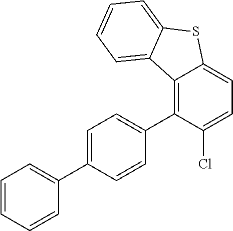 [SMILES: Clc1ccc2sc3ccccc3c2c1-c1ccc(-c2ccccc2)cc1] IM-A14 | 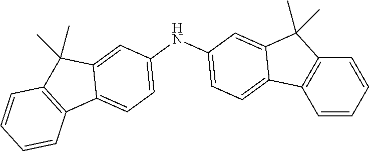 [SMILES: CC1(C)c2ccccc2-c2ccc(Nc3ccc4c(c3)C(C)(C)c3ccccc3-4)cc21] IM-B7 | 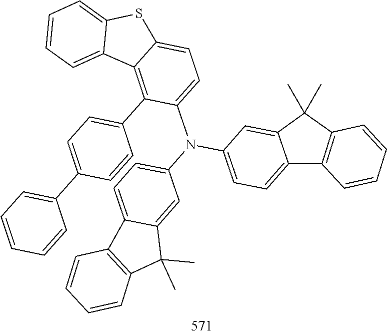 [SMILES: CC1(C)c2ccccc2-c2ccc(N(c3ccc4c(c3)C(C)(C)c3ccccc3-4)c3ccc4sc5ccccc5c4c3-c3ccc(-c4ccccc4)cc3)cc21] | 37.7 | 736.3 |
| 70 | 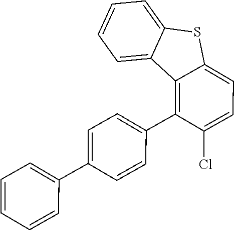 [SMILES: Clc1ccc2sc3ccccc3c2c1-c1ccc(-c2ccccc2)cc1] IM-A14 | 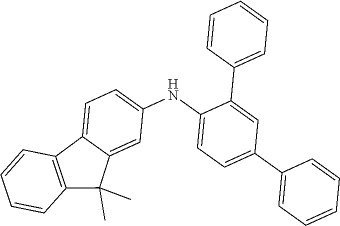 [SMILES: CC1(C)c2ccccc2-c2ccc(Nc3ccc(-c4ccccc4)cc3-c3ccccc3)cc21] IM-B17 | 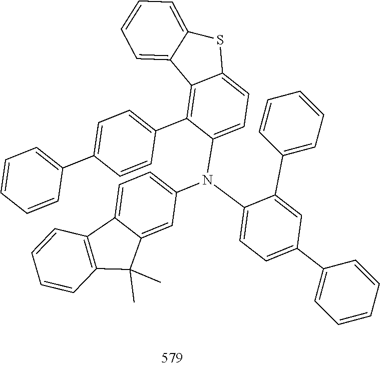 [SMILES: CC1(C)c2ccccc2-c2ccc(N(c3ccc(-c4ccccc4)cc3-c3ccccc3)c3ccc4sc5ccccc5c4c3-c3ccc(-c4ccccc4)cc3)cc21] | 45.6 | 772.3 |
| 71 | 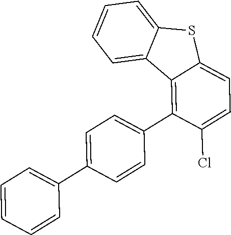 [SMILES: Clc1ccc2sc3ccccc3c2c1-c1ccc(-c2ccccc2)cc1] IM-A14 | 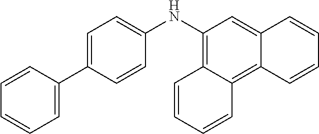 [SMILES: c1ccc(-c2ccc(Nc3cc4ccccc4c4ccccc34)cc2)cc1] IM-B33 | 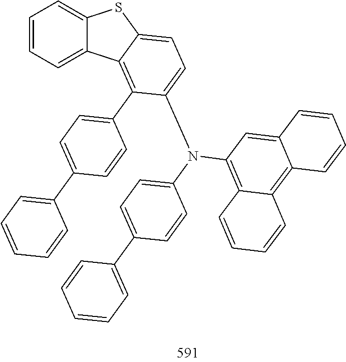 [SMILES: c1ccc(-c2ccc(-c3c(CN(c4ccc(-c5ccccc5)cc4)c4cc5ccccc5c5ccccc45)ccc4sc5ccccc5c34)cc2)cc1] | 49.5 | 680.2 |
| 72 | 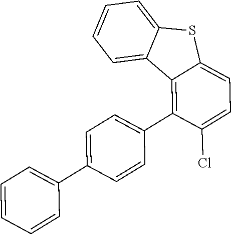 [SMILES: Clc1ccc2sc3ccccc3c2c1-c1ccc(-c2ccccc2)cc1] IM-A14 | 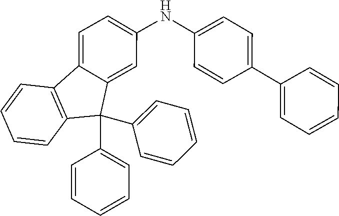 [SMILES: c1ccc(-c2ccc(Nc3ccc4c(c3)C(c3ccccc3)(c3ccccc3)c3ccccc3-4)cc2)cc1] IM-B12 | 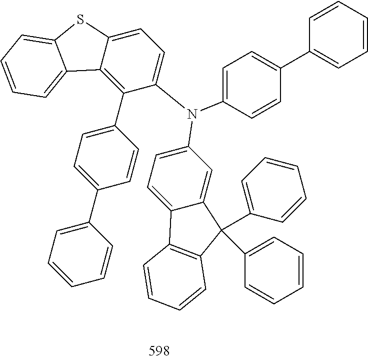 [SMILES: c1ccc(-c2ccc(-c3c(N(c4ccc(-c5ccccc5)cc4)c4ccc5c(c4)C(c4ccccc4)(c4ccccc4)c4ccccc4-5)ccc4sc5ccccc5c34)cc2)cc1] | 37.2 | 820.3 |
| 73 | 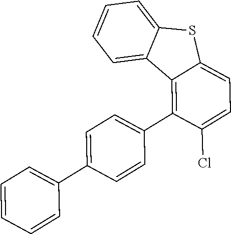 [SMILES: Clc1ccc2sc3ccccc3c2c1-c1ccc(-c2ccccc2)cc1] IM-A14 | 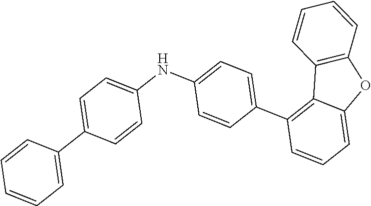 [SMILES: c1ccc(-c2ccc(Nc3ccc(-c4cccc5oc6ccccc6c45)cc3)cc2)cc1] IM-B39 | 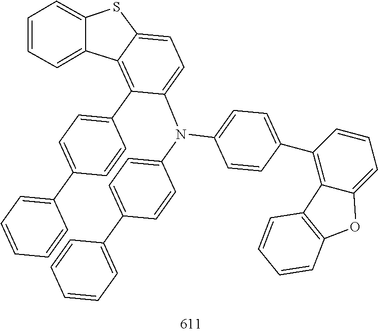 [SMILES: c1ccc(-c2ccc(-c3c(N(c4ccc(-c5ccccc5)cc4)c4ccc(-c5cccc6oc7ccccc7c56)cc4)ccc4sc5ccccc5c34)cc2)cc1] | 40.0 | 746.2 |
| 74 | 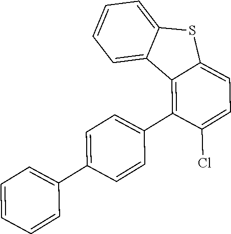 [SMILES: Clc1ccc2sc3ccccc3c2c1-c1ccc(-c2ccccc2)cc1] IM-A14 | 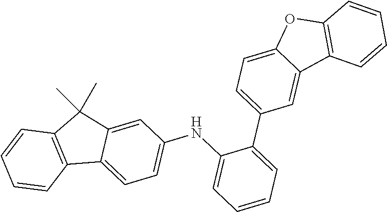 [SMILES: CC1(C)c2ccccc2-c2ccc(Nc3ccccc3-c3ccc4oc5ccccc5c4c3)cc21] IM-B34 | 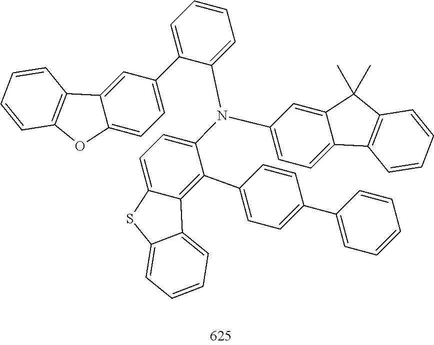 [SMILES: CC1(C)c2ccccc2-c2ccc(N(c3ccccc3-c3ccc4oc5ccccc5c4c3)c3ccc4sc5ccccc5c4c3-c3ccc(-c4ccccc4)cc3)cc21] | 41.9 | 786.3 |
| 75 | 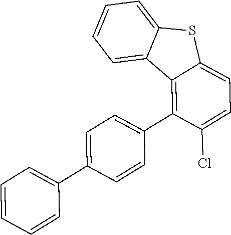 [SMILES: Clc1ccc2sc3ccccc3c2c1-c1ccc(-c2ccccc2)cc1] IM-A14 | 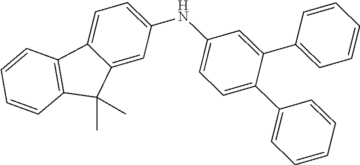 [SMILES: CC1(C)c2ccccc2-c2ccc(Nc3ccc(-c4ccccc4)c(-c4ccccc4)c3)cc21] IM-B28 | 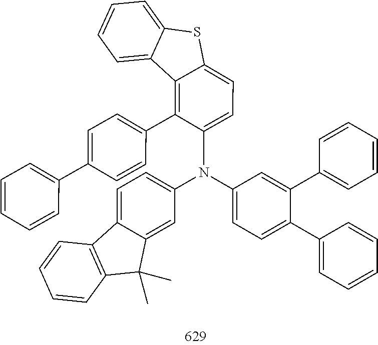 [SMILES: CC1(C)c2ccccc2-c2ccc(N(c3ccc(-c4ccccc4)c(-c4ccccc4)c3)c3ccc4sc5ccccc5c4c3-c3ccc(-c4ccccc4)cc3)cc21] | 39.5 | 772.3 |
| 76 | 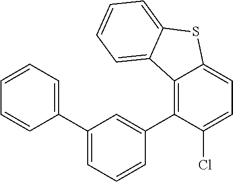 [SMILES: Clc1ccc2sc3ccccc3c2c1-c1cccc(-c2ccccc2)c1] IM-A2 | 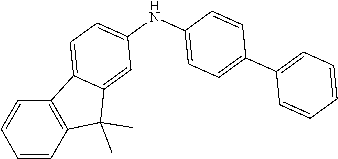 [SMILES: CC1(C)c2ccccc2-c2ccc(Nc3ccc(-c4ccccc4)cc3)cc21] IM-B2 | 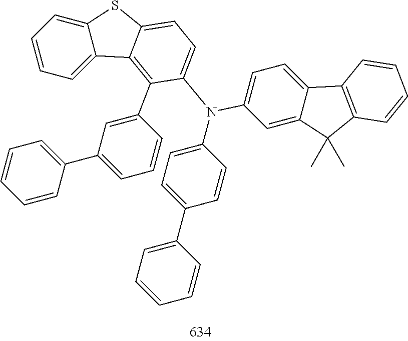 [SMILES: CC1(C)c2ccccc2-c2ccc(N(c3ccc(-c4ccccc4)cc3)c3ccc4sc5ccccc5c4c3-c3cccc(-c4ccccc4)c3)cc21] | 40.8 | 696.3 |
| 77 | 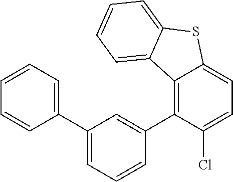 [SMILES: Clc1ccc2sc3ccccc3c2c1-c1cccc(-c2ccccc2)c1] IM-A2 | 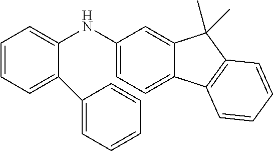 [SMILES: CC1(C)c2ccccc2-c2ccc(Nc3ccccc3-c3ccccc3)cc21] IM-B26 | 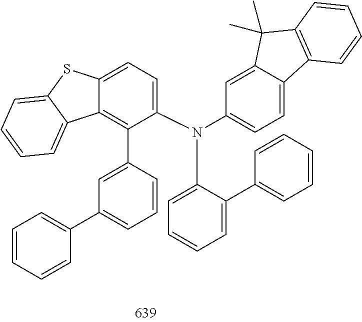 [SMILES: CC1(C)c2ccccc2-c2ccc(N(c3ccccc3-c3ccccc3)c3ccc4sc5ccccc5c4c3-c3cccc(-c4ccccc4)c3)cc21] | 40.1 | 696.3 |
| 78 | 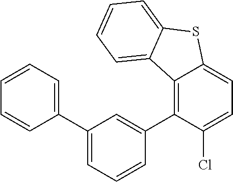 [SMILES: Clc1ccc2sc3ccccc3c2c1-c1cccc(-c2ccccc2)c1] IM-A2 | 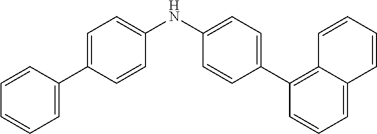 [SMILES: c1ccc(-c2ccc(Nc3ccc(-c4cccc5ccccc45)cc3)cc2)cc1] IM-B35 | 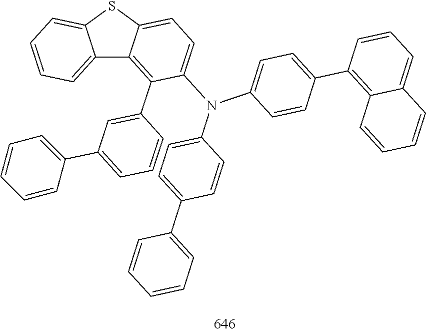 [SMILES: c1ccc(-c2ccc(N(c3ccc(-c4cccc5ccccc45)cc3)c3ccc4sc5ccccc5c4c3-c3cccc(-c4ccccc4)c3)cc2)cc1] | 43.8 | 706.2 |
| 79 | 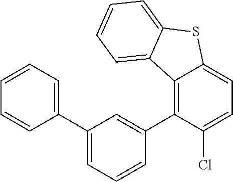 [SMILES: Clc1ccc2sc3ccccc3c2c1-c1cccc(-c2ccccc2)c1] IM-A2 | 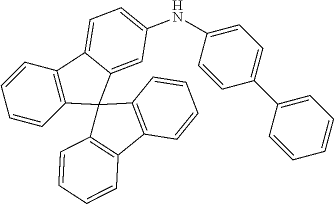 [SMILES: c1ccc(-c2ccc(Nc3ccc4c(c3)C3(c5ccccc5-c5ccccc53)c3ccccc3-4)cc2)cc1] IM-B32 | 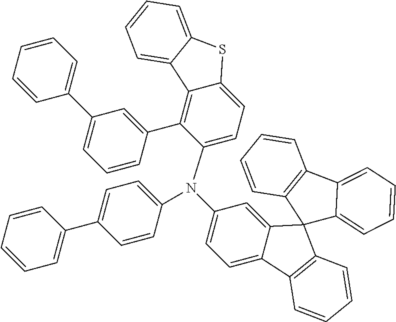 [SMILES: c1ccc(-c2ccc(N(c3ccc4c(c3)C3(c5ccccc5-c5ccccc53)c3ccccc3-4)c3ccc4sc5ccccc5c4c3-c3cccc(-c4ccccc4)c3)cc2)cc1] 658 | 40.0 | 818.3 |
| 80 | 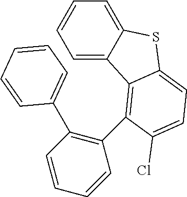 [SMILES: Clc1ccc2sc3ccccc3c2c1-c1ccccc1-c1ccccc1] IM-A15 | 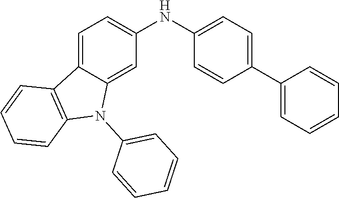 [SMILES: c1ccc(-c2ccc(Nc3ccc4c5ccccc5n(-c5ccccc5)c4c3)cc2)cc1] IM-B20 | 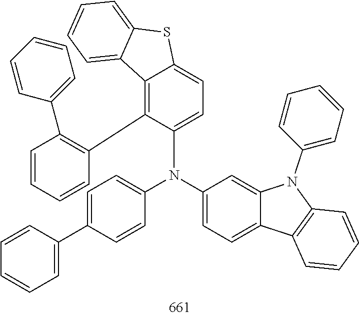 [SMILES: c1ccc(-c2ccc(N(c3ccc4c5ccccc5n(-c5ccccc5)c4c3)c3ccc4sc5ccccc5c4c3-c3ccccc3-c3ccccc3)cc2)cc1] | 41.8 | 745.2 |
| 81 | 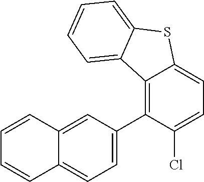 [SMILES: Clc1ccc2sc3ccccc3c2c1-c1ccc2ccccc2c1] IM-A16 | 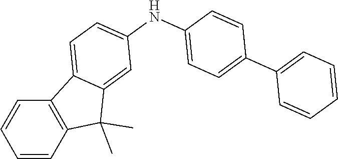 [SMILES: CC1(C)c2ccccc2-c2ccc(Nc3ccc(-c4ccccc4)cc3)cc21] IM-B2 | 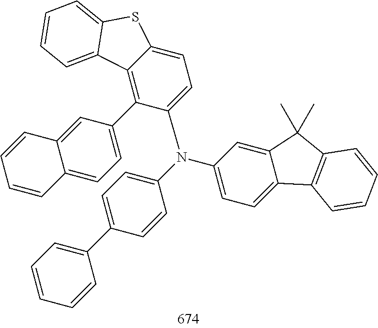 [SMILES: CC1(C)c2ccccc2-c2ccc(N(c3ccc(-c4ccccc4)cc3)c3ccc4sc5ccccc5c4c3-c3ccc4ccccc4c3)cc21] | 45.9 | 670.2 |
| 82 | 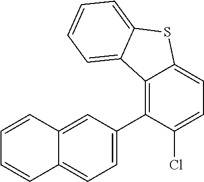 [SMILES: Clc1ccc2sc3ccccc3c2c1-c1ccc2ccccc2c1] IM-A16 | 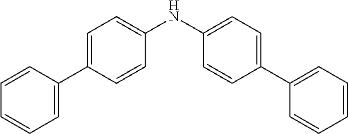 [SMILES: c1ccc(-c2ccc(Nc3ccc(-c4ccccc4)cc3)cc2)cc1] IM-B5 | 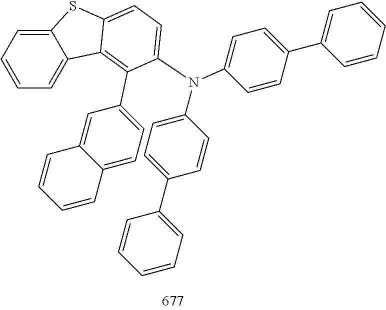 [SMILES: c1ccc(-c2ccc(N(c3ccc(-c4ccccc4)cc3)c3ccc4sc5ccccc5c4c3-c3ccc4ccccc4c3)cc2)cc1] | 48.4 | 630.2 |
| 83 | 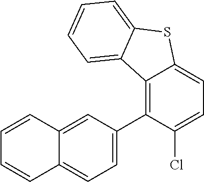 [SMILES: Clc1ccc2sc3ccccc3c2c1-c1ccc2ccccc2c1] IM-A16 | 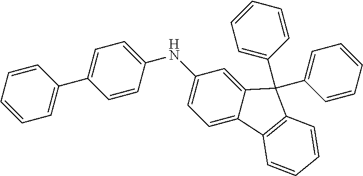 [SMILES: c1ccc(-c2ccc(Nc3ccc4c(c3)C(c3ccccc3)(c3ccccc3)c3ccccc3-4)cc2)cc1] IM-B12 | 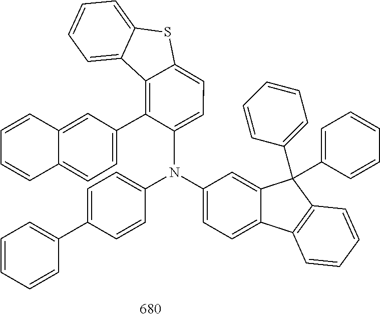 [SMILES: c1ccc(-c2ccc(N(c3ccc4c(c3)C(c3ccccc3)(c3ccccc3)c3ccccc3-4)c3ccc4sc5ccccc5c4c3-c3ccc4ccccc4c3)cc2)cc1] | 47.1 | 794.3 |
| 84 | 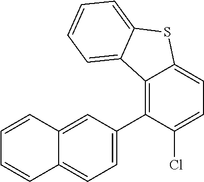 [SMILES: Clc1ccc2sc3ccccc3c2c1-c1ccc2ccccc2c1] IM-A16 | 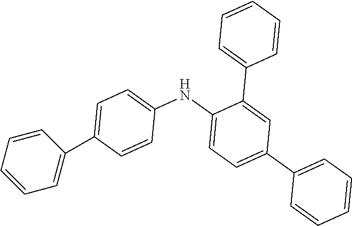 [SMILES: c1ccc(-c2ccc(Nc3ccc(-c4ccccc4)cc3-c3ccccc3)cc2)cc1] IM-B36 | 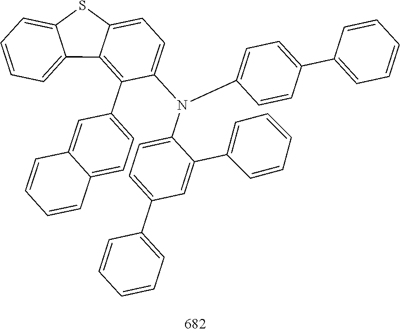 [SMILES: c1ccc(-c2ccc(N(c3ccc(-c4ccccc4)cc3-c3ccccc3)c3ccc4sc5ccccc5c4c3-c3ccc4ccccc4c3)cc2)cc1] | 35.2 | 706.2 |
| 85 | 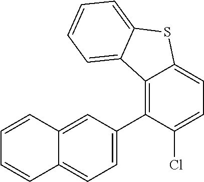 [SMILES: Clc1ccc2sc3ccccc3c2c1-c1ccc2ccccc2c1] IM-A16 | 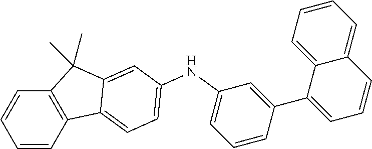 [SMILES: CC1(C)c2ccccc2-c2ccc(Nc3cccc(-c4cccc5ccccc45)c3)cc21] IM-B37 | 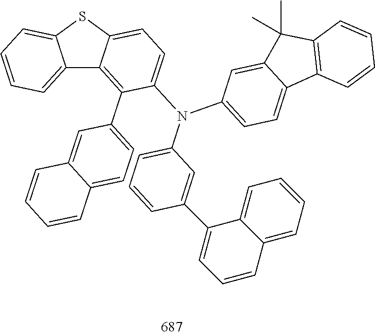 [SMILES: CC1(C)c2ccccc2-c2ccc(N(c3cccc(-c4cccc5ccccc45)c3)c3ccc4sc5ccccc5c4c3-c3ccc4ccccc4c3)cc21] | 41.6 | 720.3 |
| 86 | 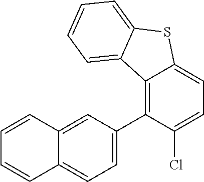 [SMILES: Clc1ccc2sc3ccccc3c2c1-c1ccc2ccccc2c1] IM-A16 | 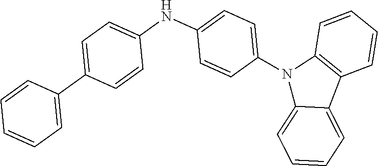 [SMILES: c1ccc(-c2ccc(Nc3ccc(-n4c5ccccc5c5ccccc54)cc3)cc2)cc1] IM-B38 | 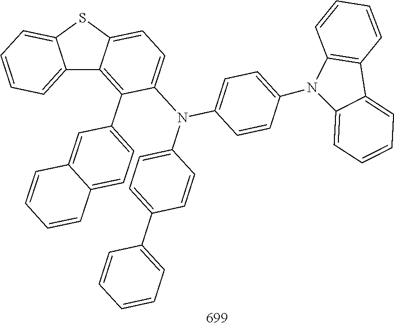 [SMILES: c1ccc(-c2ccc(N(c3ccc(-n4c5ccccc5c5ccccc54)cc3)c3ccc4sc5ccccc5c4c3-c3ccc4ccccc4c3)cc2)cc1] | 47.9 | 719.2 |
| 87 | 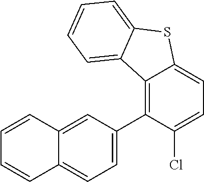 [SMILES: Clc1ccc2sc3ccccc3c2c1-c1ccc2ccccc2c1] IM-A16 | 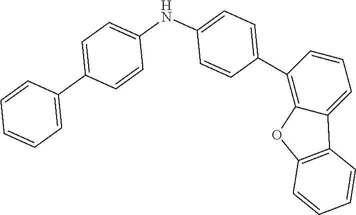 [SMILES: c1ccc(-c2ccc(Nc3ccc(-c4cccc5c4oc4ccccc45)cc3)cc2)cc1] IM-B27 | 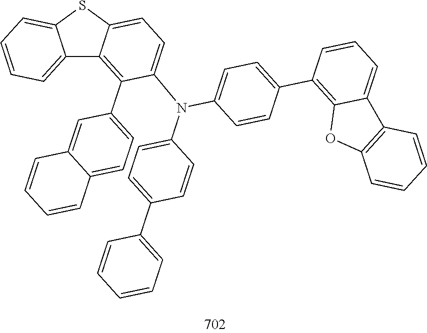 [SMILES: c1ccc(-c2ccc(N(c3ccc(-c4cccc5c4oc4ccccc45)cc3)c3ccc4sc5ccccc5c4c3-c3ccc4ccccc4c3)cc2)cc1] | 42.0 | 720.2 |
| 88 | 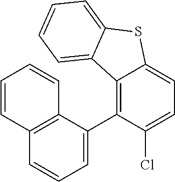 [SMILES: Clc1ccc2sc3ccccc3c2c1-c1cccc2ccccc12] IM-A17 | 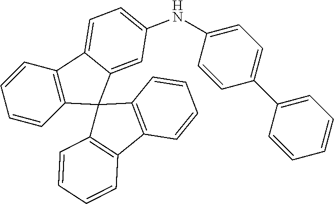 [SMILES: c1ccc(-c2ccc(Nc3ccc4c(c3)C3(c5ccccc5-c5ccccc53)c3ccccc3-4)cc2)cc1] IM-B32 | 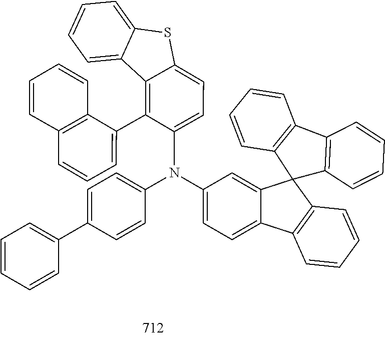 [SMILES: c1ccc(-c2ccc(N(c3ccc4c(c3)C3(c5ccccc5-c5ccccc53)c3ccccc3-4)c3ccc4sc5ccccc5c4c3-c3cccc4ccccc34)cc2)cc1] | 47.1 | 792.2 |
| 89 | 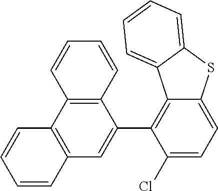 [SMILES: Clc1ccc2sc3ccccc3c2c1-c1cc2ccccc2c2ccccc12] IM-A18 | 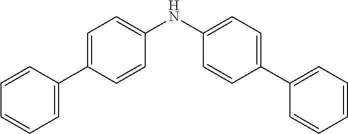 [SMILES: c1ccc(-c2ccc(Nc3ccc(-c4ccccc4)cc3)cc2)cc1] IM-B5 | 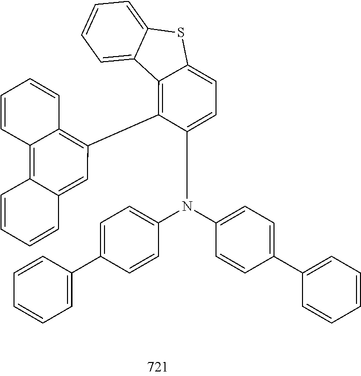 [SMILES: c1ccc(-c2ccc(N(c3ccc(-c4ccccc4)cc3)c3ccc4sc5ccccc5c4c3-c3cc4ccccc4c4ccccc34)cc2)cc1] | 48.6 | 680.2 |
| 90 | 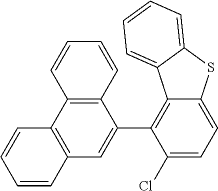 [SMILES: Clc1ccc2sc3ccccc3c2c1-c1cc2ccccc2c2ccccc12] IM-A18 | 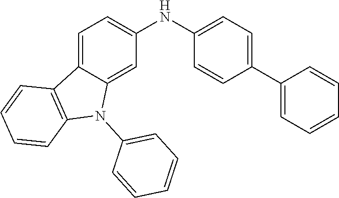 [SMILES: c1ccc(-c2ccc(Nc3ccc4c5ccccc5n(-c5ccccc5)c4c3)cc2)cc1] IM-B20 | 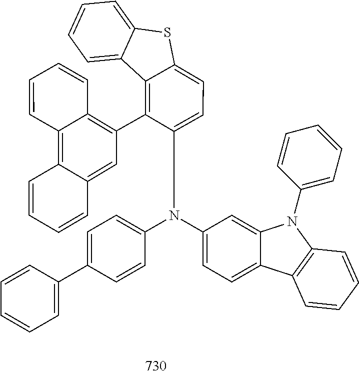 [SMILES: c1ccc(-c2ccc(N(c3ccc4c5ccccc5n(-c5ccccc5)c4c3)c3ccc4sc5ccccc5c4c3-c3cc4ccccc4c4ccccc34)cc2)cc1] | 46.8 | 769.2 |
| 91 | 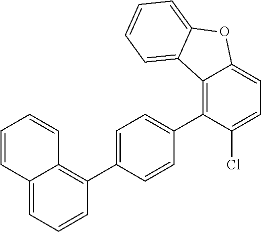 [SMILES: Clc1ccc2oc3ccccc3c2c1-c1ccc(-c2cccc3ccccc23)cc1] IM-A19 | 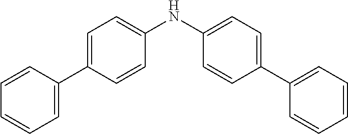 [SMILES: c1ccc(-c2ccc(Nc3ccc(-c4ccccc4)cc3)cc2)cc1] IM-B5 | 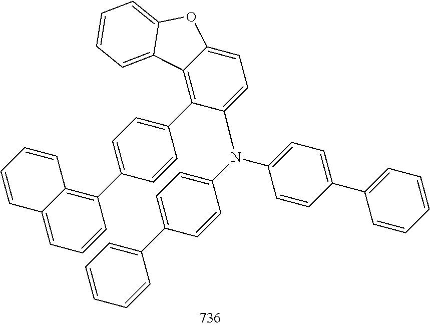 [SMILES: c1ccc(-c2ccc(N(c3ccc(-c4ccccc4)cc3)c3ccc4oc5ccccc5c4c3-c3ccc(-c4cccc5ccccc45)cc3)cc2)cc1] | 47.8 | 690.3 |
| 92 | 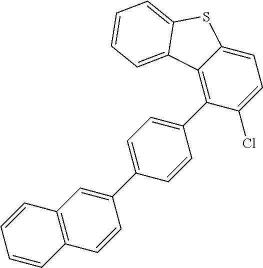 [SMILES: Clc1ccc2sc3ccccc3c2c1-c1ccc(-c2ccc3ccccc3c2)cc1] IM-A20 | 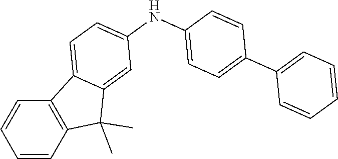 [SMILES: CC1(C)c2ccccc2-c2ccc(Nc3ccc(-c4ccccc4)cc3)cc21] IM-B2 | 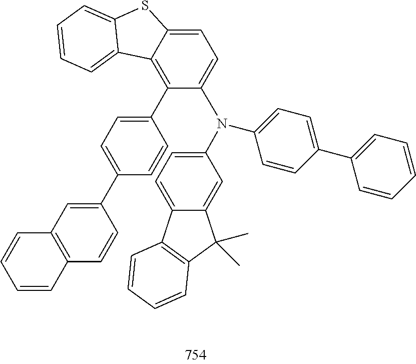 [SMILES: CC1(C)c2ccccc2-c2ccc(N(c3ccc(-c4ccccc4)cc3)c3ccc4sc5ccccc5c4c3-c3ccc(-c4ccc5ccccc5c4)cc3)cc21] | 48.4 | 746.3 |
| 93 | 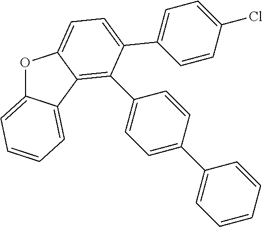 [SMILES: Clc1ccc(-c2ccc3oc4ccccc4c3c2-c2ccc(-c3ccccc3)cc2)cc1] IM-A21 | 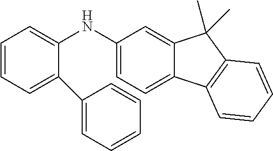 [SMILES: CC1(C)c2ccccc2-c2ccc(Nc3ccccc3-c3ccccc3)cc21] IM-B26 | 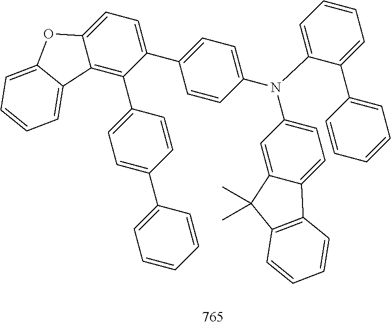 [SMILES: CC1(C)c2ccccc2-c2ccc(N(c3ccc(-c4ccc5oc6ccccc6c5c4-c4ccc(-c5ccccc5)cc4)cc3)c3ccccc3-c3ccccc3)cc21] | 43.5 | 756.3 |
| 94 | 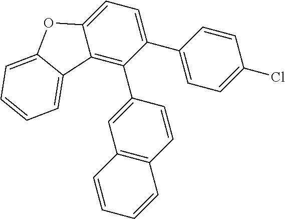 [SMILES: Clc1ccc(-c2ccc3oc4ccccc4c3c2-c2ccc3ccccc3c2)cc1] IM-A22 | 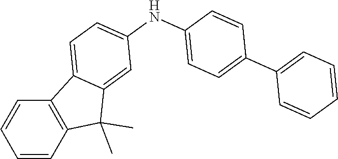 [SMILES: CC1(C)c2ccccc2-c2ccc(Nc3ccc(-c4ccccc4)cc3)cc21] IM-B2 | 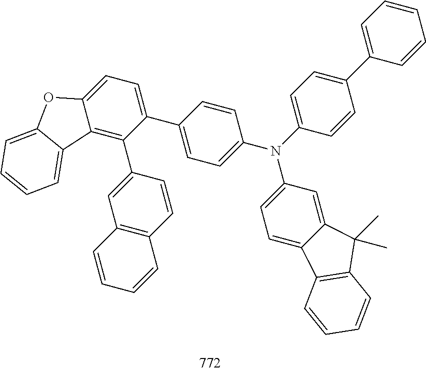 [SMILES: CC1(C)c2ccccc2-c2ccc(N(c3ccc(-c4ccccc4)cc3)c3ccc(-c4ccc5oc6ccccc6c5c4-c4ccc5ccccc5c4)cc3)cc21] | 40.2 | 730.3 |
| 95 | 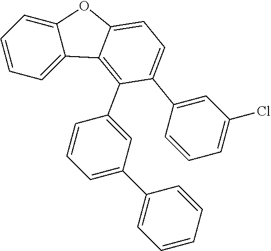 [SMILES: Clc1cccc(-c2ccc3oc4ccccc4c3c2-c2cccc(-c3ccccc3)c2)c1] IM-A23 | 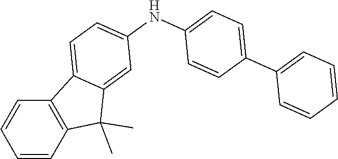 [SMILES: CC1(C)c2ccccc2-c2ccc(Nc3ccc(-c4ccccc4)cc3)cc21] IM-B2 | 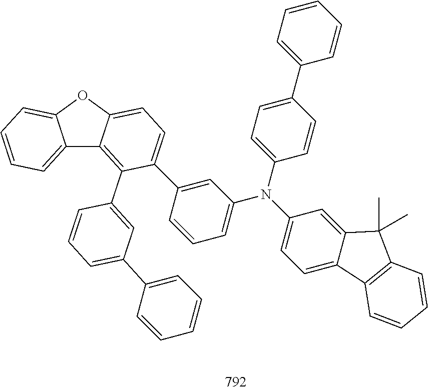 [SMILES: CC1(C)c2ccccc2-c2ccc(N(c3ccc(-c4ccccc4)cc3)c3cccc(-c4ccc5oc6ccccc6c5c4-c4cccc(-c5ccccc5)c4)c3)cc21] | 41.6 | 756.3 |
| 96 | 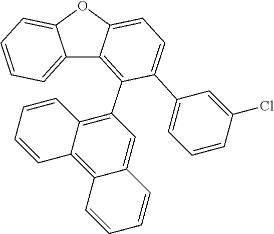 [SMILES: Clc1cccc(-c2ccc3oc4ccccc4c3c2-c2cc3ccccc3c3ccccc23)c1] IM-A24 | 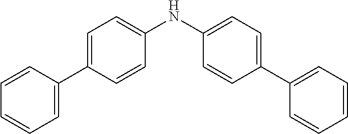 [SMILES: c1ccc(-c2ccc(Nc3ccc(-c4ccccc4)cc3)cc2)cc1] IM-B5 | 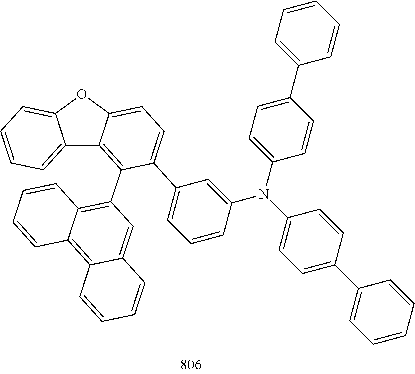 [SMILES: c1ccc(-c2ccc(N(c3ccc(-c4ccccc4)cc3)c3cccc(-c4ccc5oc6ccccc6c5c4-c4cc5ccccc5c5ccccc45)c3)cc2)cc1] | 42.1 | 740.3 |
| 97 | 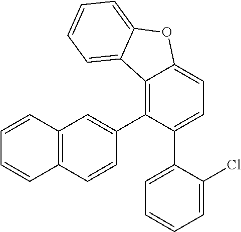 [SMILES: Clc1ccccc1-c1ccc2oc3ccccc3c2c1-c1ccc2ccccc2c1] IM-A25 | 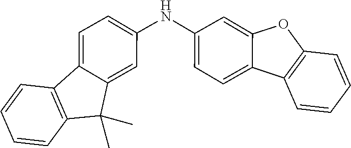 [SMILES: CC1(C)c2ccccc2-c2ccc(Nc3ccc4c(c3)oc3ccccc34)cc21] IM-B13 | 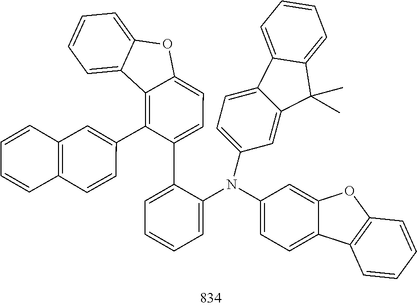 [SMILES: CC1(C)c2ccccc2-c2ccc(N(c3ccc4c(c3)oc3ccccc34)c3ccccc3-c3ccc4oc5ccccc5c4c3-c3ccc4ccccc4c3)cc21] | 34.9 | 744.3 |
| 98 | 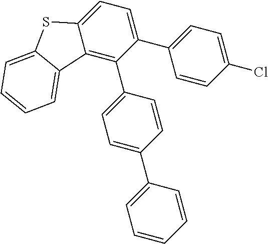 [SMILES: Clc1ccc(-c2ccc3sc4ccccc4c3c2-c2ccc(-c3ccccc3)cc2)cc1] IM-A26 | 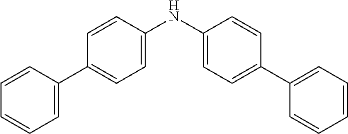 [SMILES: c1ccc(-c2ccc(Nc3ccc(-c4ccccc4)cc3)cc2)cc1] IM-B5 | 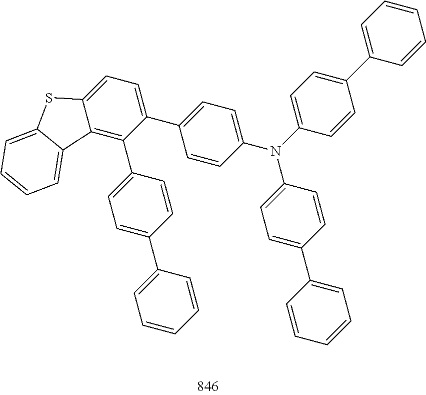 [SMILES: c1ccc(-c2ccc(-c3c(-c4ccc(N(c5ccc(-c6ccccc6)cc5)c5ccc(-c6ccccc6)cc5)cc4)ccc4sc5ccccc5c34)cc2)cc1] | 50.5 | 732.3 |
| 99 | 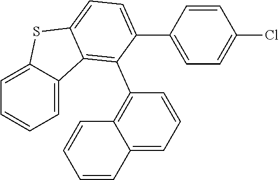 [SMILES: Clc1ccc(-c2ccc3sc4ccccc4c3c2-c2cccc3ccccc23)cc1] IM-A27 | 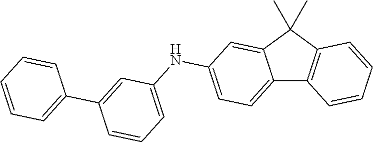 [SMILES: CC1(C)c2ccccc2-c2ccc(Nc3cccc(-c4ccccc4)c3)cc21] IM-B4 | 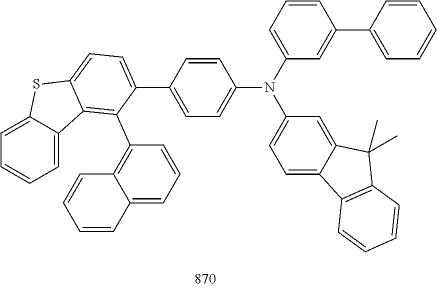 [SMILES: CC1(C)c2ccccc2-c2ccc(N(c3ccc(-c4ccc5sc6ccccc6c5c4-c4cccc5ccccc45)cc3)c3cccc(-c4ccccc4)c3)cc21] | 47.2 | 746.3 |
| 100 | 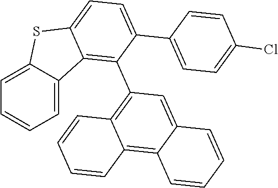 [SMILES: Clc1ccc(-c2ccc3sc4ccccc4c3c2-c2cc3ccccc3c3ccccc23)cc1] IM-A28 | 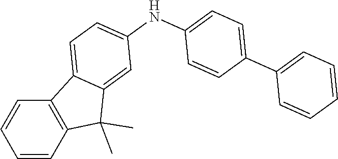 [SMILES: CC1(C)c2ccccc2-c2ccc(Nc3ccc(-c4ccccc4)cc3)cc21] IM-B2 | 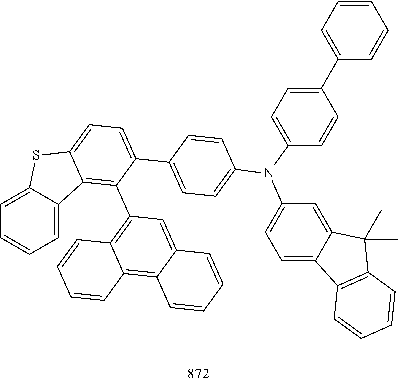 [SMILES: CC1(C)c2ccccc2-c2ccc(N(c3ccc(-c4ccccc4)cc3)c3ccc(-c4ccc5sc6ccccc6c5c4-c4cc5ccccc5c5ccccc45)cc3)cc21] | 48.9 | 796.3 |
| 101 | 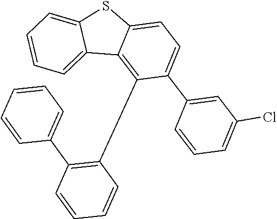 [SMILES: Clc1cccc(-c2ccc3sc4ccccc4c3c2-c2ccccc2-c2ccccc2)c1] IM-A29 | 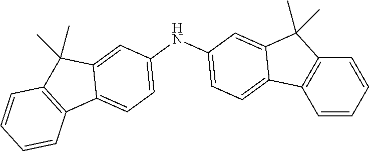 [SMILES: CC1(C)c2ccccc2-c2ccc(Nc3ccc4c(c3)C(C)(C)c3ccccc3-4)cc21] IM-B7 | 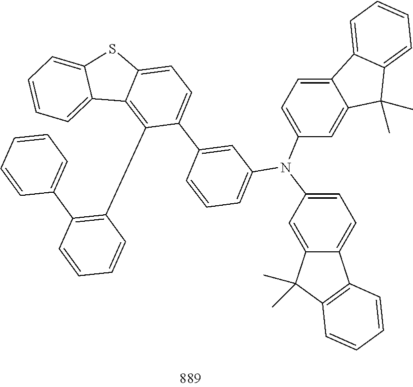 [SMILES: CC1(C)c2ccccc2-c2ccc(N(c3cccc(-c4ccc5sc6ccccc6c5c4-c4ccccc4-c4ccccc4)c3)c3ccc4c(c3)C(C)(C)c3ccccc3-4)cc21] | 47.9 | 812.3 |
| 102 | 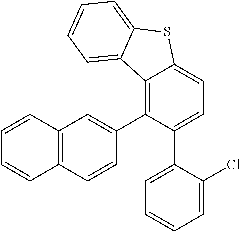 [SMILES: Clc1ccccc1-c1ccc2sc3ccccc3c2c1-c1ccc2ccccc2c1] IM-A30 | 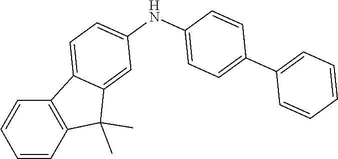 [SMILES: CC1(C)c2ccccc2-c2ccc(Nc3ccc(-c4ccccc4)cc3)cc21] IM-B2 | 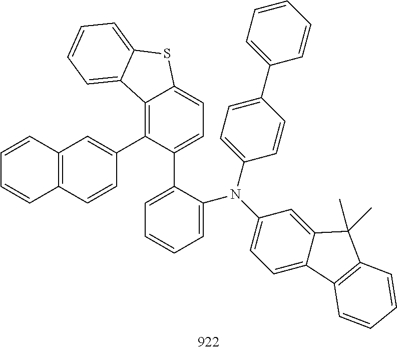 [SMILES: CC1(C)c2ccccc2-c2ccc(N(c3ccc(-c4ccccc4)cc3)c3ccccc3-c3ccc4sc5ccccc5c4c3-c3ccc4ccccc4c3)cc21] | 39.3 | 746.3 |
| 103 | 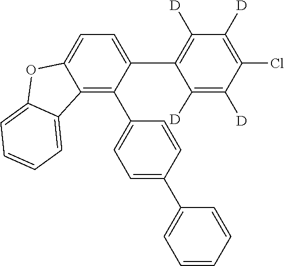 [SMILES: [2H]c1c([2H])c(-c2ccc3oc4ccccc4c3c2-c2ccc(-c3ccccc3)cc2)c([2H])c([2H])c1Cl] IM-A31 | 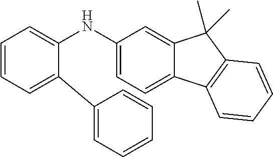 [SMILES: CC1(C)c2ccccc2-c2ccc(Nc3ccccc3-c3ccccc3)cc21] IM-B26 | 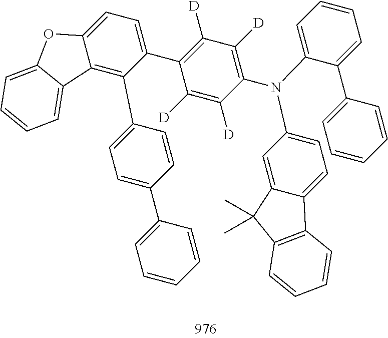 [SMILES: [2H]c1c([2H])c(N(c2ccc3c(c2)C(C)(C)c2ccccc2-3)c2ccccc2-c2ccccc2)c([2H])c([2H])c1-c1ccc2oc3ccccc3c2c1-c1ccc(-c2ccccc2)cc1] | 42.0 | 760.4 |
| 104 | 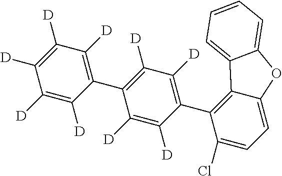 [SMILES: [2H]c1c([2H])c([2H])c(-c2c([2H])c([2H])c(-c3c(Cl)ccc4oc5ccccc5c34)c([2H])c2[2H])c([2H])c1[2H]] IM-A32 | 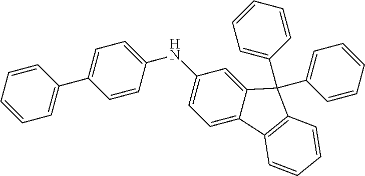 [SMILES: c1ccc(-c2ccc(Nc3ccc4c(c3)C(c3ccccc3)(c3ccccc3)c3ccccc3-4)cc2)cc1] IM-B12 | 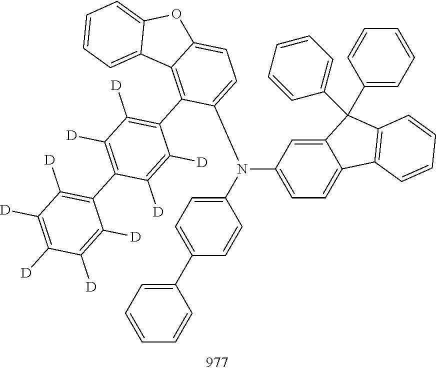 [SMILES: [2H]c1c([2H])c([2H])c(-c2c([2H])c([2H])c(-c3c(N(c4ccc(-c5ccccc5)cc4)c4ccc5c(c4)C(c4ccccc4)(c4ccccc4)c4ccccc4-5)ccc4oc5ccccc5c34)c([2H])c2[2H])c([2H])c1[2H]] | 37.5 | 813.4 |
| 105 | 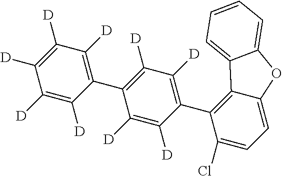 [SMILES: [2H]c1c([2H])c([2H])c(-c2c([2H])c([2H])c(-c3c(Cl)ccc4oc5ccccc5c34)c([2H])c2[2H])c([2H])c1[2H]] IM-A32 | 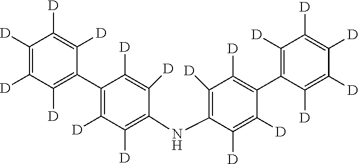 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] IM-B40 | 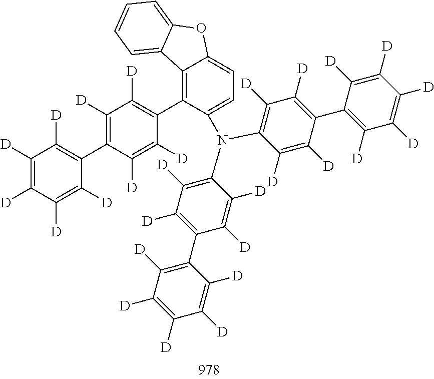 [SMILES: [2H]c1c([2H])c([2H])c(-c2c([2H])c([2H])c(-c3c(N(c4c([2H])c([2H])c(-c5c([2H])c([2H])c([2H])c([2H])c5[2H])c([2H])c4[2H])c4c([2H])c([2H])c(-c5c([2H])c([2H])c([2H])c([2H])c5[2H])c([2H])c4[2H])ccc4oc5ccccc5c34)c([2H])c2[2H])c([2H])c1[2H]] | 44.8 | 667.4 |
| 106 | 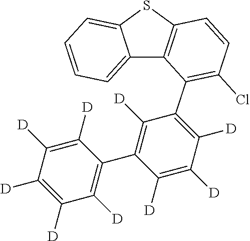 [SMILES: [2H]c1c([2H])c([2H])c(-c2c([2H])c([2H])c([2H])c(-c3c(Cl)ccc4sc5ccccc5c34)c2[2H])c([2H])c1[2H]] IM-A33 | 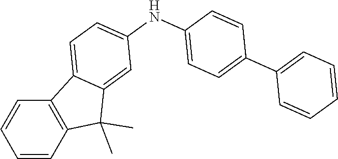 [SMILES: CC1(C)c2ccccc2-c2ccc(Nc3ccc(-c4ccccc4)cc3)cc21] IM-B2 | 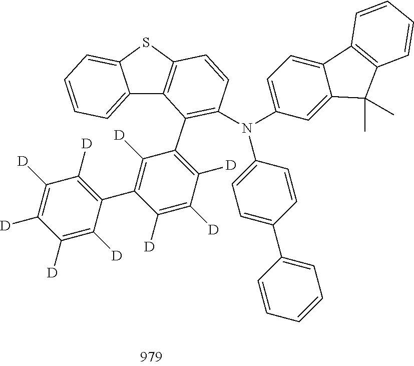 [SMILES: [2H]c1c([2H])c([2H])c(-c2c([2H])c([2H])c([2H])c(-c3c(N(c4ccc(-c5ccccc5)cc4)c4ccc5c(c4)C(C)(C)c4ccccc4-5)ccc4sc5ccccc5c34)c2[2H])c([2H])c1[2H]] | 40.3 | 705.3 |
| 107 | 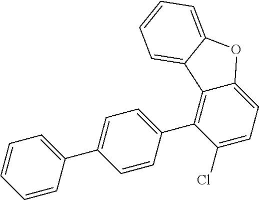 [SMILES: Clc1ccc2oc3ccccc3c2c1-c1ccc(-c2ccccc2)cc1] IM-A1 | 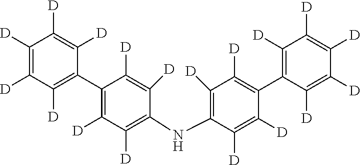 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] IM-B40 | 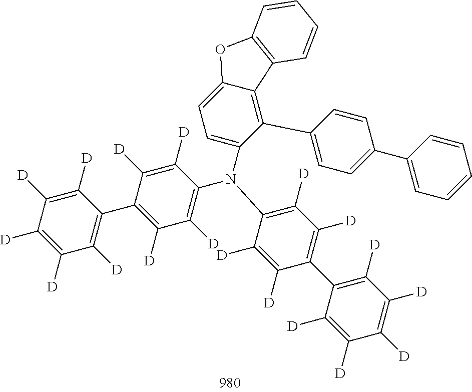 [SMILES: [2H]c1c([2H])c([2H])c(-c2c([2H])c([2H])c(N(c3ccc4oc5ccccc5c4c3-c3ccc(-c4ccccc4)cc3)c3c([2H])c([2H])c(-c4c([2H])c([2H])c([2H])c([2H])c4[2H])c([2H])c3[2H])c([2H])c2[2H])c([2H])c1[2H]] | 43.2 | 758.4 |

### NMR Data of Some Compounds:

Compound 21 NMR: ¹H-NMR (400 MHZ, CD₂Cl₂) δ ppm: 8.07 (d, 1H), 7.72 (d, 2H), 7.64-7.51 (m, 14H), 7.49 (d, 2H), 7.45-7.39 (m, 7H), 7.35 (t, 1H), 7.28 (d, 1H), 6.86 (d, 1H), 6.62 (d, 4H).

Compound 229 NMR: ¹H-NMR (400 MHZ, CD₂Cl₂) δ ppm: 8.06 (d, 1H), 7.95-7.82 (m, 5H), 7.74 (d, 1H), 7.57-7.47 (m, 9H), 7.45-7.39 (m, 4H), 7.34 (t, 1H), 7.31 (d, 1H), 7.15-7.08 (m, 2H), 6.82 (d, 2H), 6.77 (d, 1H), 6.72 (d, 1H), 6.58 (s, 1H), 1.46 (s, 6H).

Compound 870 NMR: ¹H-NMR (400 MHZ, CD₂Cl₂) δ ppm: 8.21 (d, 1H), 8.04 (d, 1H), 7.99-7.93 (m, 3H), 7.85 (d, 1H), 7.82 (d, 1H), 7.65 (t, 1H), 7.57-7.51 (m, 6H), 7.49-7.43 (m, 4H), 7.39 (d, 2H), 7.33-7.29 (m, 2H), 7.17-7.12 (m, 3H), 7.07 (d, 2H), 7.05 (s, 1H), 6.85-6.82 (m, 3H), 6.72 (d, 1H), 6.59 (s, 1H), 1.48 (s, 6H).

### Manufacture and Performance Evaluation of Organic Electroluminescent Devices:

### Example 1: Manufacture of Red Organic Electroluminescent Device

First, an anode was pre-treated through the following process: surface treatment was performed on ITO/Ag/ITO substrates with thicknesses of 100 Å, 1000 Å, and 100 Å in sequence using ultraviolet ozone and O₂:N₂ plasma to increase the work function of the anode, or the surface of an ITO substrate was cleaned with an organic solvent to remove impurities and oil stains therefrom.

The compound HAT-CN was evaporated in vacuum on the experimental substrate (anode) to form a hole injection layer (HIL) with a thickness of 105 Å, and then the compound HT-01 was evaporated in vacuum on the hole injection layer to form a hole transport layer (HTL) with a thickness of 1135 Å.

The compound 9 was evaporated in vacuum on the hole transport layer to form a hole adjustment layer (also known as a hole auxiliary layer) with a thickness of 80 Å. Next, on the hole adjustment layer, the compound RH-01 and the compound RD-01 were co-evaporated at a rate ratio of 95%:5% to form a red electroluminescent layer (EML) with a thickness of 300 Å.

The compound HB-01 was evaporated in vacuum on the electroluminescent layer to form a hole blocking layer (HBL) with a thickness of 500 Å. Then, on the hole blocking layer, the compound ET-01 and LiQ were mixed in a weight ratio of 1:1 and evaporated to form an electron transport layer (ETL) with a thickness of 300 Å, Yb was evaporated on the electron transport layer to form an electron injection layer (EIL) with a thickness of 15 Å, and magnesium (Mg) and silver (Ag) were evaporated together on the electron injection layer at a rate of 1:9 to form a cathode with a thickness of 10 Å.

In addition, the compound CP-01 with a thickness of 600 Å was evaporated in vacuum on the cathode as a cathode protective layer (CPL), thereby completing the manufacture of an organic electroluminescent device.

### Examples 2 to 107

Organic electroluminescent devices were manufactured using the same method as Example 1, except that the compounds in Table 5 were used instead of compound 9 in Example 1 when the hole assist layer was manufactured.

### Comparative Examples 1 to 5

Organic electroluminescent devices were manufactured using the same method as Example 1, except that compounds A to E were used instead of compound 9 in Example 1 when the hole assist layer was manufactured.

When the organic electroluminescent devices were fabricated, the structures of various materials used in the comparative examples and examples were as follows:

The main material structures used in the above examples and comparative examples were as follows.

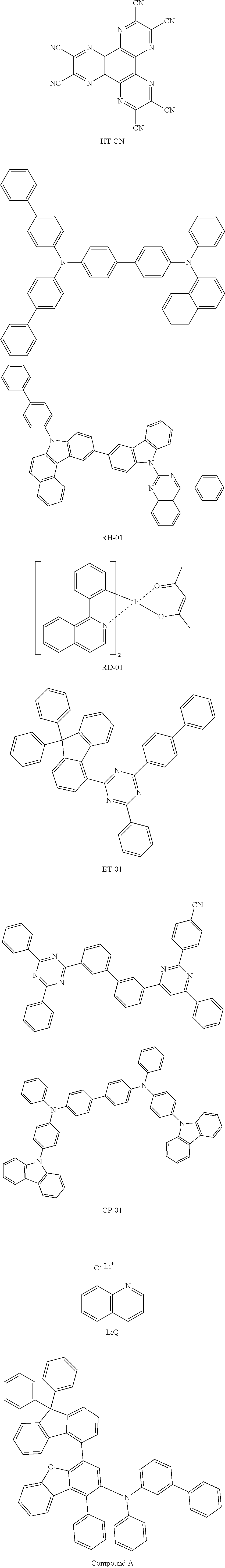

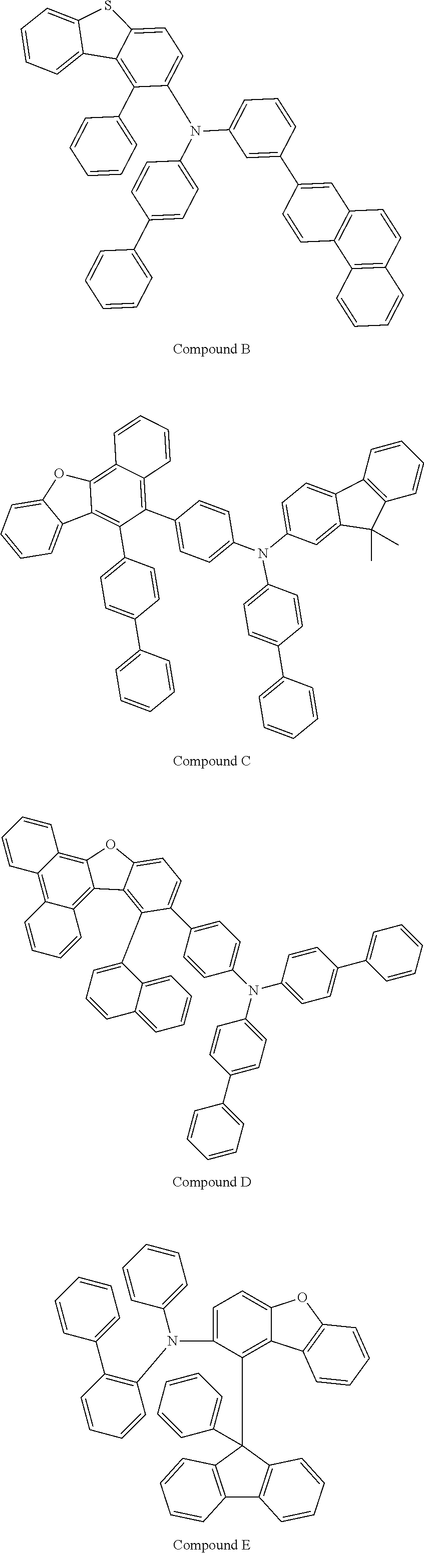 [SMILES: c1ccc(-c2ccc(N(c3cccc(-c4ccc5c(ccc6ccccc65)c4)c3)c3ccc4sc5ccccc5c4c3-c3ccccc3)cc2)cc1 ; CC1(C)c2ccccc2-c2ccc(N(c3ccc(-c4ccccc4)cc3)c3ccc(-c4c(-c5ccc(-c6ccccc6)cc5)c5c6ccccc6oc5c5ccccc45)cc3)cc21 ; c1ccc(-c2ccc(N(c3ccc(-c4ccccc4)cc3)c3ccc(-c4ccc5oc6c7ccccc7c7ccccc7c6c5c4-c4cccc5ccccc45)cc3)cc2)cc1 ; c1ccc(-c2ccccc2N(c2ccccc2)c2ccc3oc4ccccc4c3c2C2(c3ccccc3)c3ccccc3-c3ccccc32)cc1]

Performances of the organic electroluminescent devices fabricated in Examples 1 to 107 and Comparative Examples 1 to 5 were tested. Specifically, IVL performances of the devices were tested under the condition of 10 mA/cm², and T95 lifetimes of the devices were tested under the condition of 15 mA/cm². The test results were shown in Table 5 below:

TABLE 5

| | | | | | | T95 |
|---|---|---|---|---|---|---|
| | Hole | Working | | | | (15 |
| | auxiliary | voltage | | | | mA/ |
| Example | layer | (V) | Cd/A | CIEx | CIEy | cm²) |
| Example 1 | Compound 9 | 3.97 | 59.6 | 0.68 | 0.32 | 455.1 |
| Example 2 | Compound 5 | 4.04 | 61.9 | 0.68 | 0.32 | 448.3 |
| Example 3 | Compound 15 | 3.99 | 62.1 | 0.68 | 0.32 | 446.2 |
| Example 4 | Compound 19 | 3.94 | 60.8 | 0.68 | 0.32 | 449.3 |
| Example 5 | Compound 21 | 3.99 | 61.0 | 0.68 | 0.32 | 468.7 |
| Example 6 | Compound 24 | 4.07 | 58.1 | 0.68 | 0.32 | 456.5 |
| Example 7 | Compound 31 | 4.03 | 58.5 | 0.68 | 0.32 | 454.2 |
| Example 8 | Compound 37 | 4.01 | 62.2 | 0.68 | 0.32 | 450.2 |
| Example 9 | Compound 40 | 3.98 | 59.2 | 0.68 | 0.32 | 451.5 |
| Example 10 | Compound 46 | 4.07 | 60.4 | 0.68 | 0.32 | 453.1 |
| Example 11 | Compound 53 | 4.01 | 60.7 | 0.68 | 0.32 | 454.9 |
| Example 12 | Compound 58 | 4.05 | 60.6 | 0.68 | 0.32 | 460.7 |
| Example 13 | Compound 64 | 3.98 | 58.8 | 0.68 | 0.32 | 467.2 |
| Example 14 | Compound 66 | 4.03 | 59.5 | 0.68 | 0.32 | 465.1 |
| Example 15 | Compound 78 | 3.95 | 61.0 | 0.68 | 0.32 | 455.9 |
| Example 16 | Compound 99 | 4.05 | 61.5 | 0.68 | 0.32 | 452.7 |
| Example 17 | Compound 116 | 4.01 | 60.1 | 0.68 | 0.32 | 457.3 |
| Example 18 | Compound 121 | 4.03 | 59.2 | 0.68 | 0.32 | 462.2 |
| Example 19 | Compound 129 | 3.92 | 58.5 | 0.68 | 0.32 | 463.9 |
| Example 20 | Compound 148 | 3.97 | 58.8 | 0.68 | 0.32 | 464.8 |
| Example 21 | Compound 154 | 4.01 | 59.7 | 0.68 | 0.32 | 457.6 |
| Example 22 | Compound 171 | 3.98 | 59.8 | 0.68 | 0.32 | 454.5 |
| Example 23 | Compound 184 | 4.03 | 61.1 | 0.68 | 0.32 | 428.2 |
| Example 24 | Compound 188 | 3.92 | 60.4 | 0.68 | 0.32 | 436.4 |
| Example 25 | Compound 191 | 4.01 | 59.6 | 0.68 | 0.32 | 430.4 |
| Example 26 | Compound 196 | 4.00 | 61.3 | 0.68 | 0.32 | 433.5 |
| Example 27 | Compound 207 | 3.94 | 61.8 | 0.68 | 0.32 | 436.2 |
| Example 28 | Compound 229 | 4.02 | 61.4 | 0.68 | 0.32 | 462.6 |
| Example 29 | Compound 235 | 3.98 | 59.3 | 0.68 | 0.32 | 460.1 |
| Example 30 | Compound 239 | 4.04 | 59.6 | 0.68 | 0.32 | 463.5 |
| Example 31 | Compound 241 | 4.00 | 59.0 | 0.68 | 0.32 | 463.6 |
| Example 32 | Compound 246 | 3.96 | 60.7 | 0.68 | 0.32 | 447.4 |
| Example 33 | Compound 251 | 4.05 | 60.0 | 0.68 | 0.32 | 448.6 |
| Example 34 | Compound 260 | 4.04 | 60.6 | 0.68 | 0.32 | 459.7 |
| Example 35 | Compound 268 | 3.99 | 60.2 | 0.68 | 0.32 | 458.4 |
| Example 36 | Compound 938 | 4.01 | 61.7 | 0.68 | 0.32 | 462.9 |
| Example 37 | Compound 943 | 3.95 | 58.2 | 0.68 | 0.32 | 456.7 |
| Example 38 | Compound 950 | 4.01 | 60.9 | 0.68 | 0.32 | 460.4 |
| Example 39 | Compound 273 | 4.00 | 58.5 | 0.68 | 0.32 | 458.5 |
| Example 40 | Compound 281 | 3.96 | 61.2 | 0.68 | 0.32 | 456.7 |
| Example 41 | Compound 291 | 4.03 | 60.0 | 0.68 | 0.32 | 439.1 |
| Example 42 | Compound 290 | 3.96 | 60.0 | 0.68 | 0.32 | 435.8 |
| Example 43 | Compound 296 | 3.97 | 59.0 | 0.68 | 0.32 | 433.3 |
| Example 44 | Compound 309 | 3.96 | 58.7 | 0.68 | 0.32 | 430.5 |
| Example 45 | Compound 340 | 4.07 | 65.1 | 0.68 | 0.32 | 375.3 |
| Example 46 | Compound 344 | 4.00 | 65.3 | 0.68 | 0.32 | 374.5 |
| Example 47 | Compound 351 | 3.94 | 65.5 | 0.68 | 0.32 | 380.7 |
| Example 48 | Compound 361 | 3.94 | 66.2 | 0.68 | 0.32 | 376.6 |
| Example 49 | Compound 376 | 3.94 | 65.7 | 0.68 | 0.32 | 373.5 |
| Example 50 | Compound 386 | 3.92 | 65.8 | 0.68 | 0.32 | 379.4 |
| Example 51 | Compound 388 | 4.09 | 65.6 | 0.68 | 0.32 | 382.3 |
| Example 52 | Compound 402 | 3.92 | 66.9 | 0.68 | 0.32 | 379.7 |
| Example 53 | Compound 419 | 4.07 | 65.0 | 0.68 | 0.32 | 381.4 |
| Example 54 | Compound 422 | 3.95 | 65.9 | 0.68 | 0.32 | 379.6 |
| Example 55 | Compound 436 | 3.96 | 66.7 | 0.68 | 0.32 | 377.3 |
| Example 56 | Compound 441 | 4.02 | 66.4 | 0.68 | 0.32 | 383.9 |
| Example 57 | Compound 450 | 3.94 | 67.4 | 0.68 | 0.32 | 378.9 |
| Example 58 | Compound 457 | 4.00 | 67.5 | 0.68 | 0.32 | 379.0 |
| Example 59 | Compound 465 | 4.02 | 66.3 | 0.68 | 0.32 | 374.2 |
| Example 60 | Compound 464 | 4.00 | 65.1 | 0.68 | 0.32 | 376.1 |
| Example 61 | Compound 487 | 4.02 | 67.2 | 0.68 | 0.32 | 370.5 |
| Example 62 | Compound 502 | 4.03 | 66.8 | 0.68 | 0.32 | 386.2 |
| Example 63 | Compound 515 | 4.09 | 60.3 | 0.68 | 0.32 | 429.5 |
| Example 64 | Compound 534 | 4.03 | 61.1 | 0.68 | 0.32 | 432.5 |
| Example 65 | Compound 536 | 4.08 | 58.7 | 0.68 | 0.32 | 430.8 |
| Example 66 | Compound 548 | 4.09 | 58.9 | 0.68 | 0.32 | 435.3 |
| Example 67 | Compound 555 | 4.03 | 59.5 | 0.68 | 0.32 | 433.2 |
| Example 68 | Compound 561 | 4.04 | 60.9 | 0.68 | 0.32 | 431.5 |
| Example 69 | Compound 571 | 4.00 | 60.3 | 0.68 | 0.32 | 429.7 |
| Example 70 | Compound 579 | 3.92 | 60.6 | 0.68 | 0.32 | 422.5 |
| Example 71 | Compound 591 | 4.02 | 59.2 | 0.68 | 0.32 | 429.1 |
| Example 72 | Compound 598 | 4.07 | 60.0 | 0.68 | 0.32 | 445.7 |
| Example 73 | Compound 611 | 3.96 | 58.7 | 0.68 | 0.32 | 429.6 |
| Example 74 | Compound 625 | 4.06 | 59.9 | 0.68 | 0.32 | 437.8 |
| Example 75 | Compound 629 | 3.97 | 61.3 | 0.68 | 0.32 | 432.5 |
| Example 76 | Compound 634 | 4.06 | 61.6 | 0.68 | 0.32 | 428.3 |
| Example 77 | Compound 639 | 3.95 | 61.3 | 0.68 | 0.32 | 428.9 |
| Example 78 | Compound 646 | 3.98 | 61.7 | 0.68 | 0.32 | 419.7 |
| Example 79 | Compound 658 | 3.93 | 62.3 | 0.68 | 0.32 | 427.5 |
| Example 80 | Compound 661 | 4.06 | 59.1 | 0.68 | 0.32 | 426.8 |
| Example 81 | Compound 674 | 3.92 | 60.2 | 0.68 | 0.32 | 423.9 |
| Example 82 | Compound 677 | 3.92 | 62.3 | 0.68 | 0.32 | 419.5 |
| Example 83 | Compound 680 | 4.03 | 58.6 | 0.68 | 0.32 | 430.9 |
| Example 84 | Compound 682 | 4.03 | 59.5 | 0.68 | 0.32 | 432.8 |
| Example 85 | Compound 687 | 4.07 | 60.7 | 0.68 | 0.32 | 427.5 |
| Example 86 | Compound 699 | 3.92 | 58.6 | 0.68 | 0.32 | 422.3 |
| Example 87 | Compound 702 | 3.98 | 59.6 | 0.68 | 0.32 | 424.9 |
| Example 88 | Compound 712 | 4.07 | 59.7 | 0.68 | 0.32 | 427.8 |
| Example 89 | Compound 721 | 4.05 | 58.1 | 0.68 | 0.32 | 425.7 |
| Example 90 | Compound 730 | 4.08 | 59.2 | 0.68 | 0.32 | 426.2 |
| Example 91 | Compound 736 | 4.05 | 59.5 | 0.68 | 0.32 | 418.1 |
| Example 92 | Compound 754 | 3.93 | 61.9 | 0.68 | 0.32 | 421.9 |
| Example 93 | Compound 765 | 3.99 | 61.2 | 0.68 | 0.32 | 449.4 |
| Example 94 | Compound 772 | 3.92 | 58.3 | 0.68 | 0.32 | 461.7 |
| Example 95 | Compound 792 | 3.92 | 62.3 | 0.68 | 0.32 | 455.6 |
| Example 96 | Compound 806 | 3.93 | 67.0 | 0.68 | 0.32 | 378.5 |
| Example 97 | Compound 834 | 4.06 | 61.5 | 0.68 | 0.32 | 456.7 |
| Example 98 | Compound 846 | 3.96 | 58.9 | 0.68 | 0.32 | 423.4 |
| Example 99 | Compound 870 | 4.08 | 58.6 | 0.68 | 0.32 | 430.3 |
| Example 100 | Compound 872 | 4.08 | 66.7 | 0.68 | 0.32 | 373.6 |
| Example 101 | Compound 889 | 4.07 | 61.3 | 0.68 | 0.32 | 420.5 |
| Example 102 | Compound 922 | 4.02 | 59.1 | 0.68 | 0.32 | 418.2 |
| Example 103 | Compound 976 | 4.01 | 61.3 | 0.68 | 0.32 | 453.0 |
| Example 104 | Compound 977 | 4.05 | 60.2 | 0.68 | 0.32 | 462.9 |
| Example 105 | Compound 978 | 4.01 | 61.1 | 0.68 | 0.32 | 475.7 |
| Example 106 | Compound 979 | 4.06 | 61.4 | 0.68 | 0.32 | 442.3 |
| Example 107 | Compound 980 | 4.0 | 61.2 | 0.68 | 0.32 | 470.7 |
| Comparative | Compound A | 4.35 | 46.3 | 0.68 | 0.32 | 303.1 |
| Example 1 | | | | | | |
| Comparative | Compound B | 4.38 | 48.2 | 0.68 | 0.32 | 316.5 |
| Example 2 | | | | | | |
| Comparative | Compound C | 4.15 | 49.6 | 0.68 | 0.32 | 327.5 |
| Example 3 | | | | | | |
| Comparative | Compound D | 4.21 | 47.2 | 0.68 | 0.32 | 324.2 |
| Example 4 | | | | | | |
| Comparative | Compound E | 4.13 | 50.3 | 0.68 | 0.32 | 320.8 |
| Example 5 | | | | | | |

From Table 5 above, it can be seen that compared to the organic electroluminescent devices in Comparative Examples 1 to 5, the performances of the organic electroluminescent devices in Examples 1 to 107 were significantly improved, mainly manifested in an increase of at least 15.5% in the luminous efficiency (Cd/A) of the devices and an increase of at least 13.13% in T95 lifetime.

The preferred embodiments of the present invention are described in detail above with reference to the accompanying drawings. However, the present invention is not limited to the specific details in the above embodiments. Various simple modifications can be made to the technical solutions of the present invention within the technical idea scope of the present invention, and these simple modifications fall within the protection scope of the present invention.

1. An aromatic amine compound, wherein the aromatic amine compound has a structure represented by formula 1: 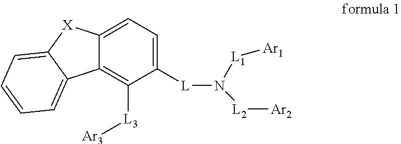
wherein X is selected from O or S;
L is selected from a single bond and substituted or unsubstituted phenylene;
L₁ and L₂ are the same or different, and are each independently selected from a single bond, substituted or unsubstituted arylene with 6 to 15 carbon atoms, and substituted or unsubstituted heteroarylene with 12 to 18 carbon atoms;
L₃ is selected from a single bond, substituted or unsubstituted phenylene, and substituted or unsubstituted naphthylene;
the substituent(s) in L, L₁, L₂, and L₃ are the same or different, and are each independently selected from deuterium, cyano, a halogen group, alkyl with 1 to 4 carbon atoms, haloalkyl with 1 to 4 carbon atoms, deuterated alkyl with 1 to 4 carbon atoms and phenyl;
Ar₁ and Ar₂ are the same or different, and are each independently selected from substituted or unsubstituted aryl with 6 to 40 carbon atoms, and substituted or unsubstituted heteroaryl with 5 to 40 carbon atoms;
Ar₃ is selected from substituted or unsubstituted biphenyl, substituted or unsubstituted terphenyl, substituted or unsubstituted naphthyl, substituted or unsubstituted phenanthryl, and substituted or unsubstituted triphenylene;
the substituent(s) in Ar₃ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms and phenyl; and
the substituent(s) in Ar₁ and Ar₂ are the same or different, and are each independently selected from deuterium, cyano, a halogen group, alkyl with 1 to 10 carbon atoms, haloalkyl with 1 to 10 carbon atoms, deuterated alkyl with 1 to 10 carbon atoms, alkoxyl with 1 to 10 carbon atoms, trialkylsilyl with 3 to 12 carbon atoms, triphenylsilyl, aryl with 6 to 20 carbon atoms, deuterated aryl with 6 to 20 carbon atoms, heteroaryl with 3 to 20 carbon atoms, cycloalkyl with 3 to 10 carbon atoms, alkylthiol with 1 to 10 carbon atoms, aryloxyl with 6 to 20 carbon atoms, and arylthiol with 6 to 20 carbon atoms; optionally, any two adjacent substituents form a 5- to 15-membered ring.

2. The aromatic amine compound according to claim 1, wherein L₁ and L₂ are the same or different, and are each independently selected from a single bond, substituted or unsubstituted phenylene, substituted or unsubstituted naphthylene, substituted or unsubstituted biphenylene, substituted or unsubstituted dibenzothienylene, substituted or unsubstituted dibenzofuranylene, and substituted or unsubstituted carbazolylene.

3. The aromatic amine compound according to claim 1, wherein L is selected from a single bond and the following groups:

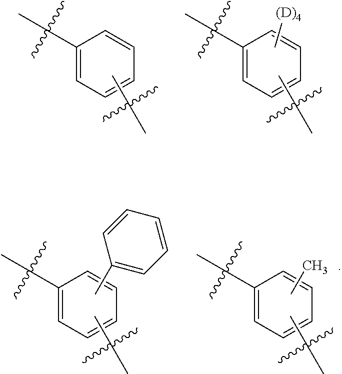

4. The aromatic amine compound according to claim 1, wherein L₃ is selected from a single bond and the following groups:

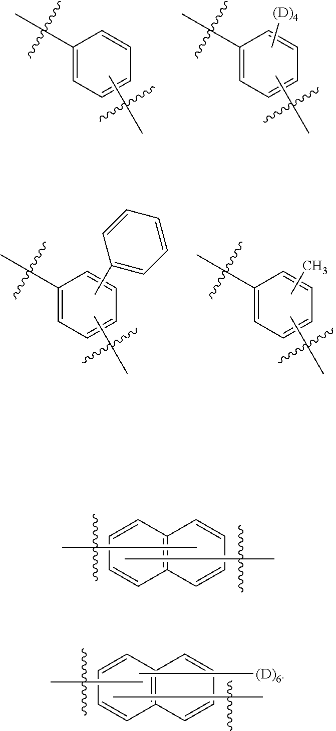

5. The aromatic amine compound according to claim 1, wherein Ar₃ is selected from the following groups:

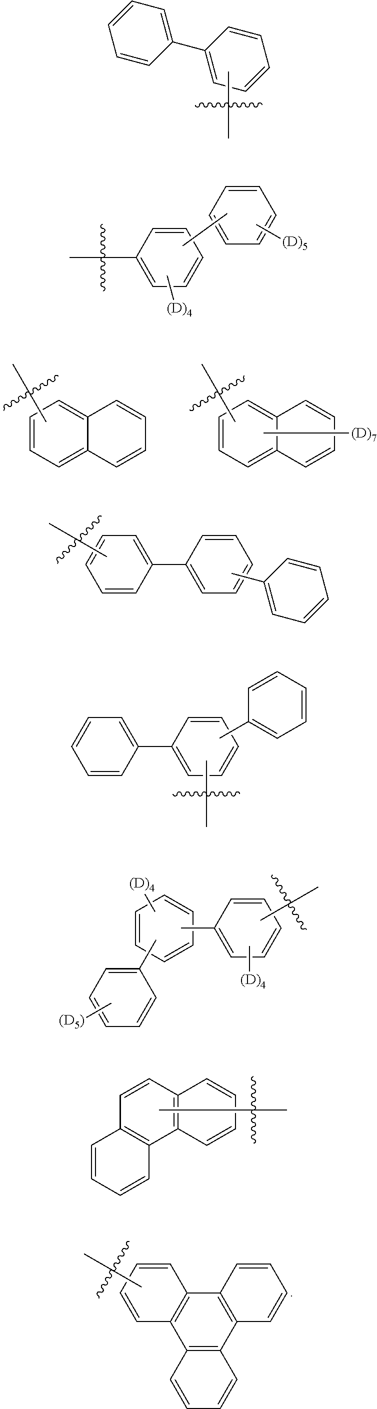

6. The aromatic amine compound according to claim 1, wherein Ar₁ and Ar₂ are the same or different, and are each independently selected from substituted or unsubstituted phenyl, substituted or unsubstituted biphenyl, substituted or unsubstituted terphenyl, substituted or unsubstituted naphthyl, substituted or unsubstituted anthryl, substituted or unsubstituted phenanthryl, substituted or unsubstituted fluorenyl, substituted or unsubstituted benzofluorenyl, substituted or unsubstituted spirobifluorenyl, substituted or unsubstituted triphenylene, substituted or unsubstituted pyrenyl, substituted or unsubstituted perylenyl, substituted or unsubstituted dibenzothienyl, substituted or unsubstituted dibenzofuranyl, substituted or unsubstituted carbazolyl, substituted or unsubstituted benzothiazolyl, substituted or unsubstituted benzoxazolyl, and substituted or unsubstituted benzimidazolyl.

7. The aromatic amine compound according to claim 1, wherein 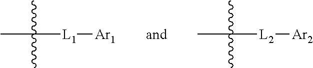
are the same or different, and are each independently selected from the following groups:

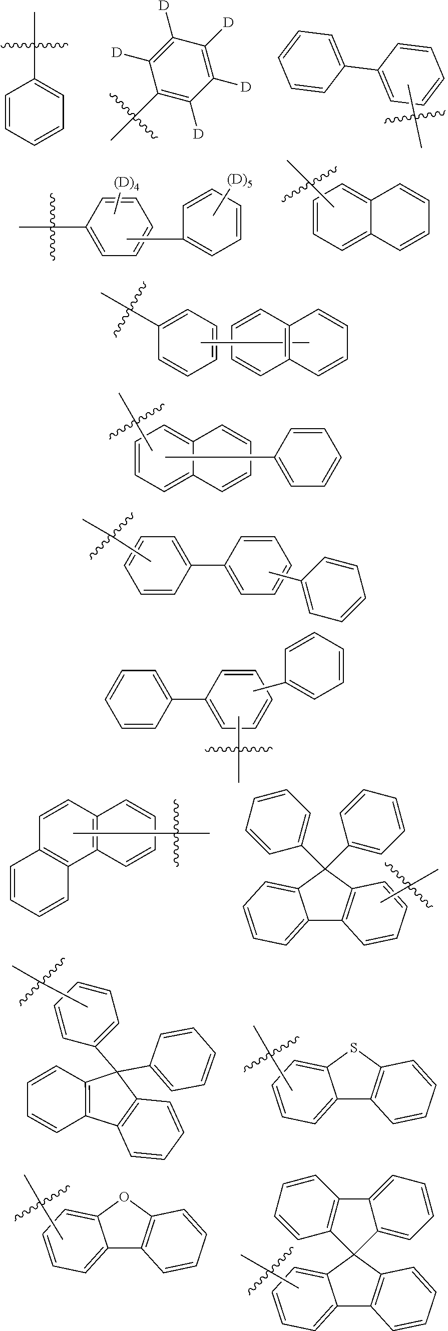

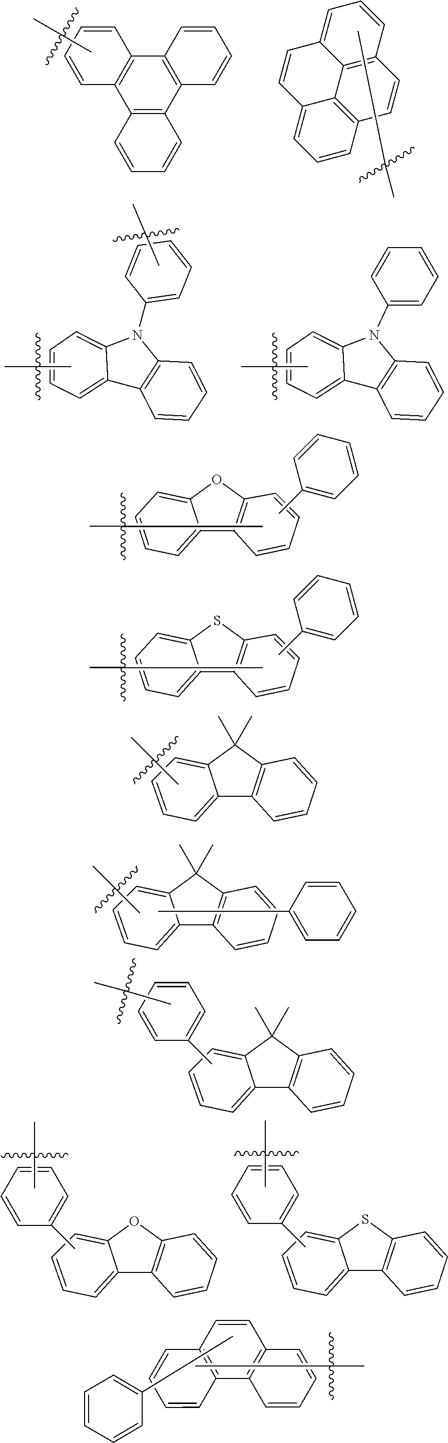

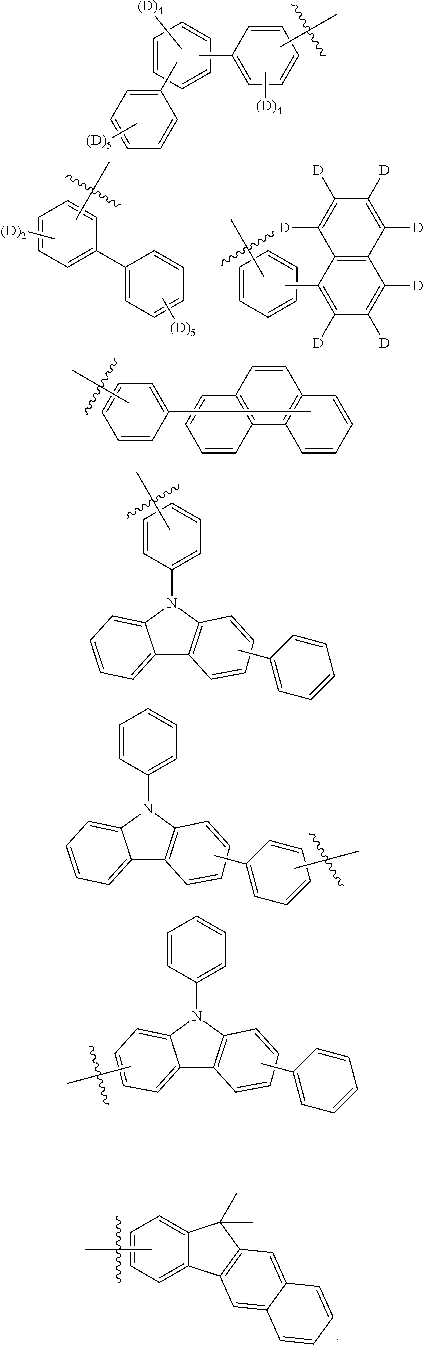

8. The aromatic amine compound according to claim 1, wherein 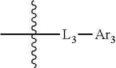
is selected from the group consisting of the following groups:

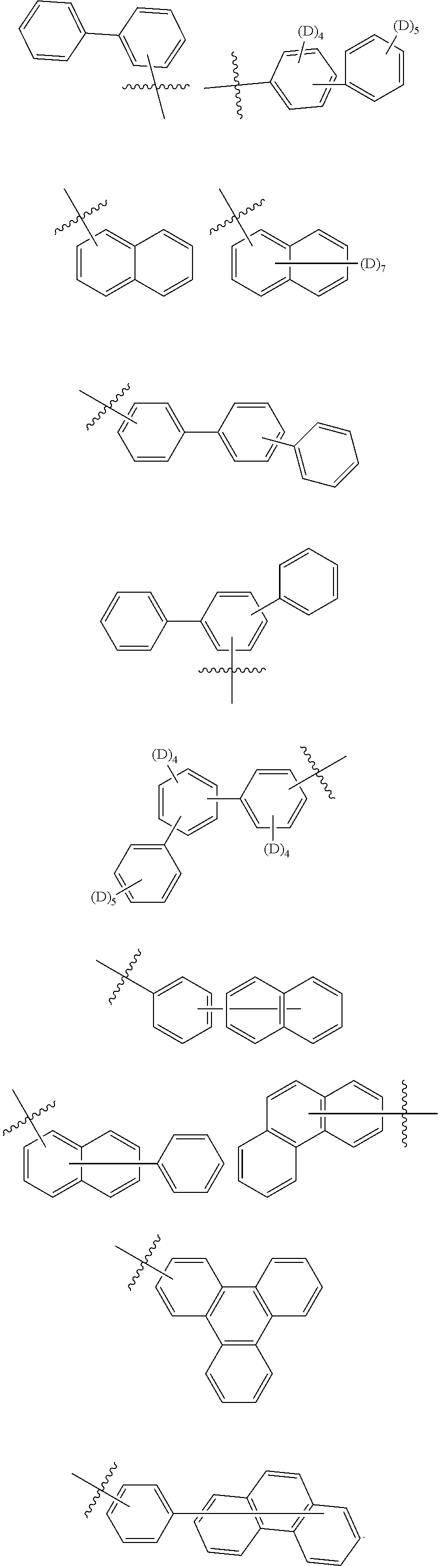

9. The aromatic amine compound according to claim 1, wherein 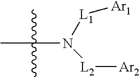
is selected from the following groups:

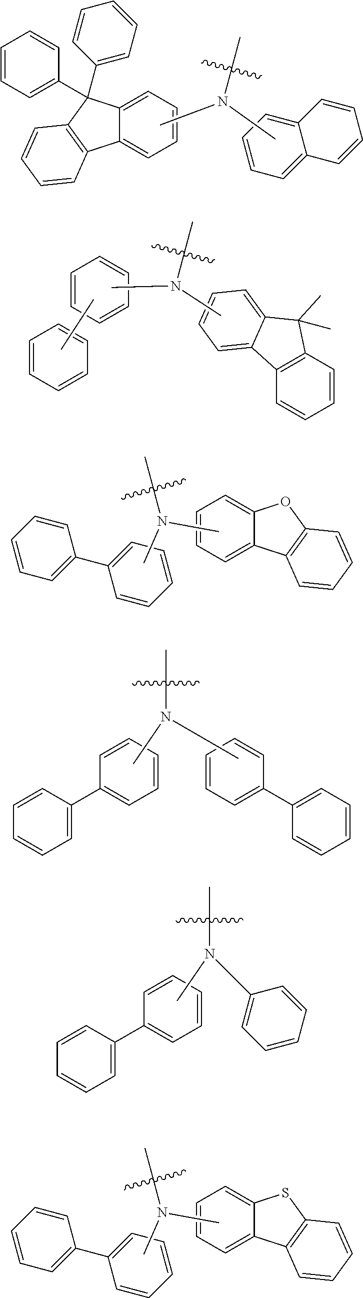

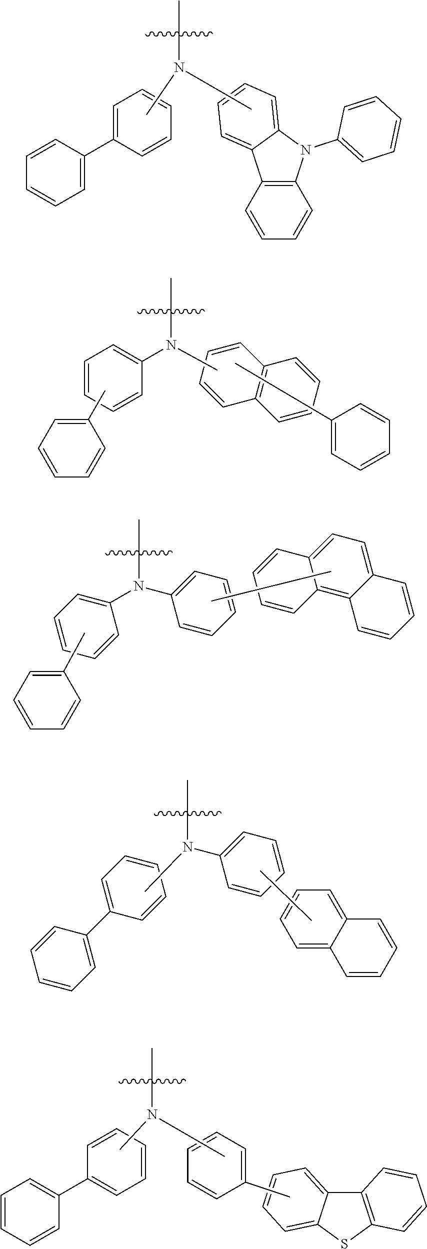

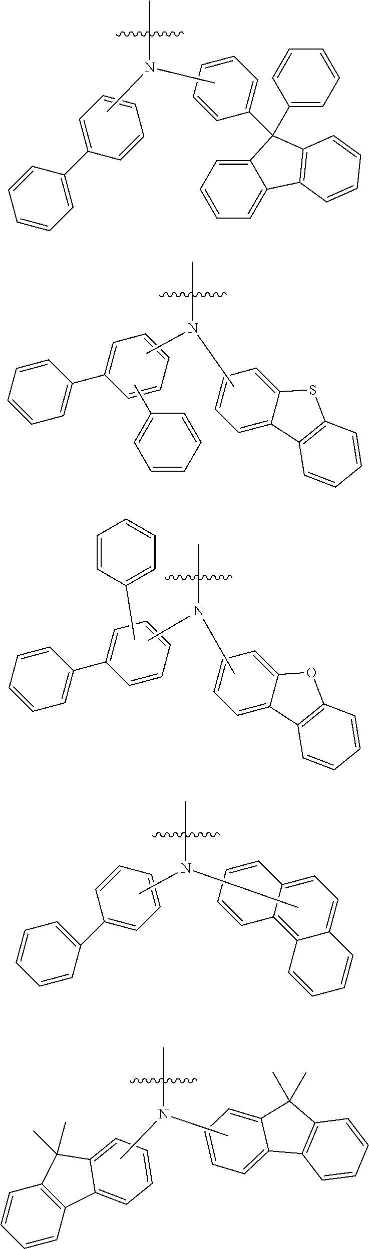

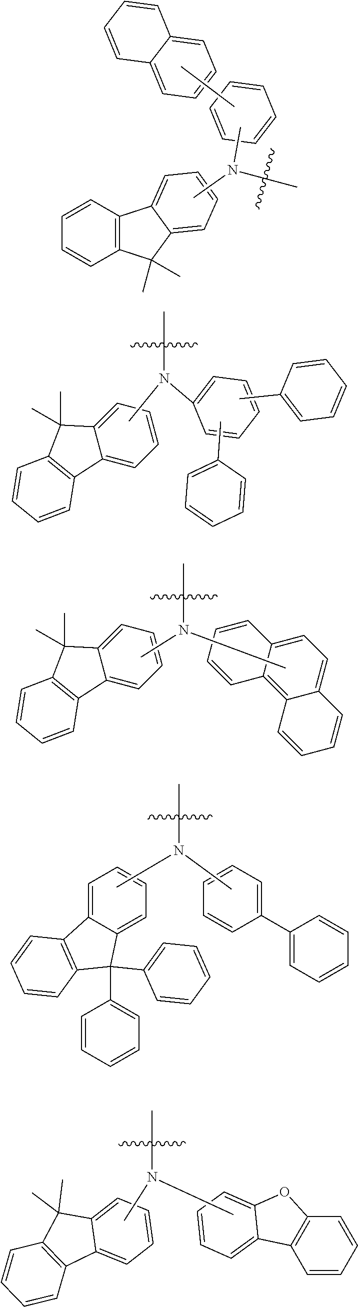

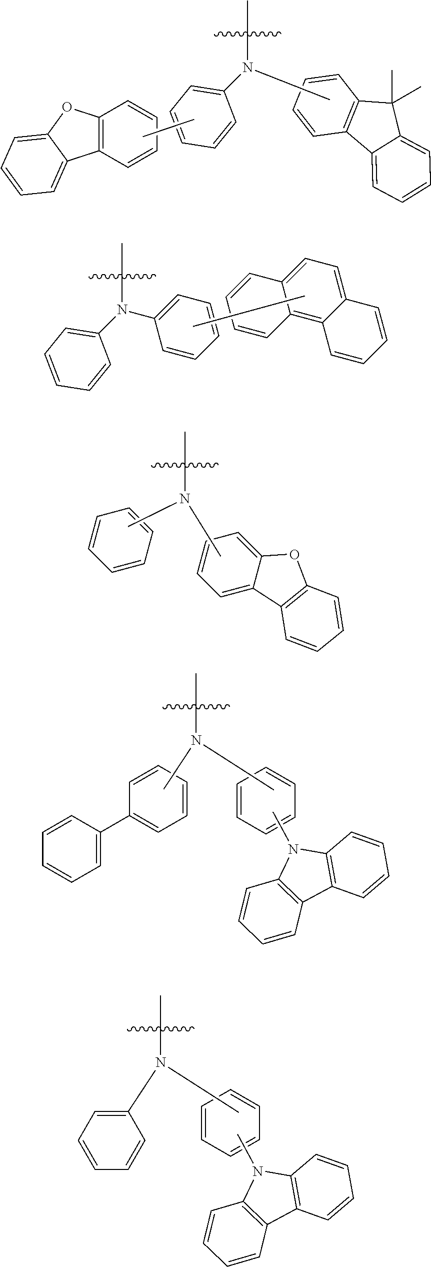

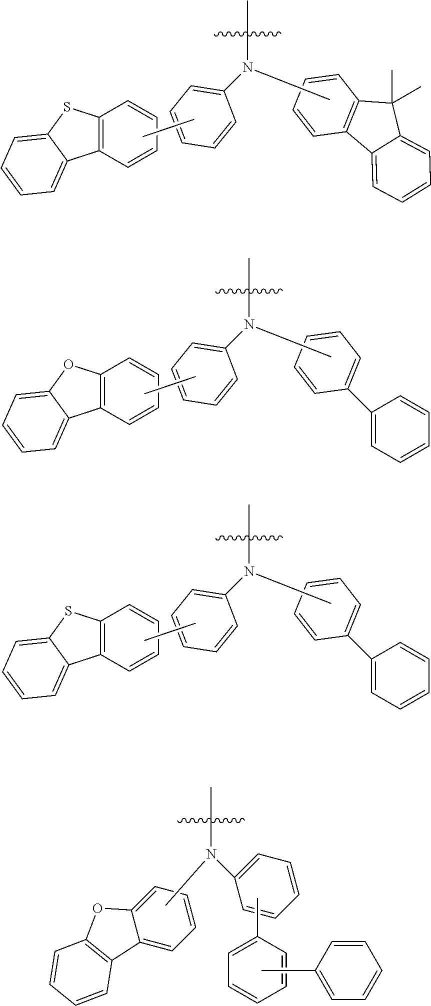

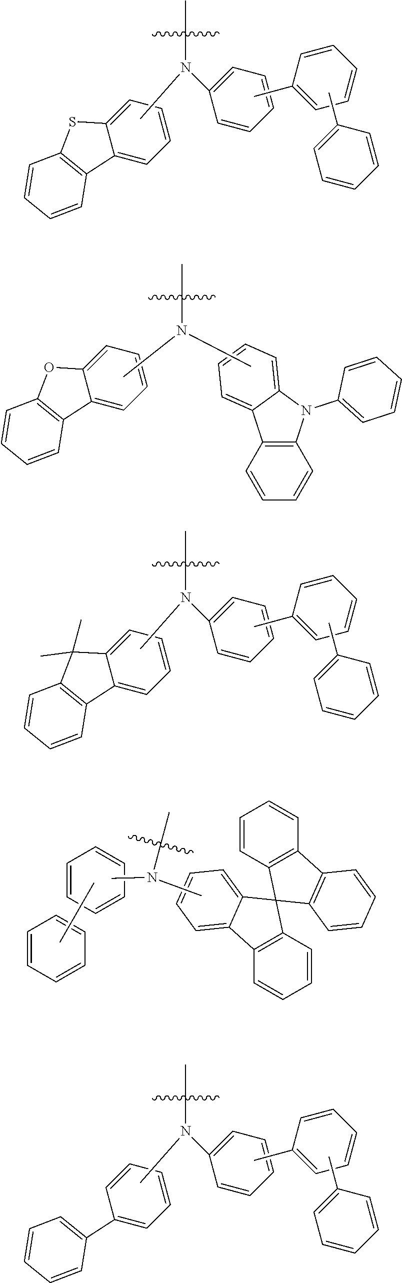

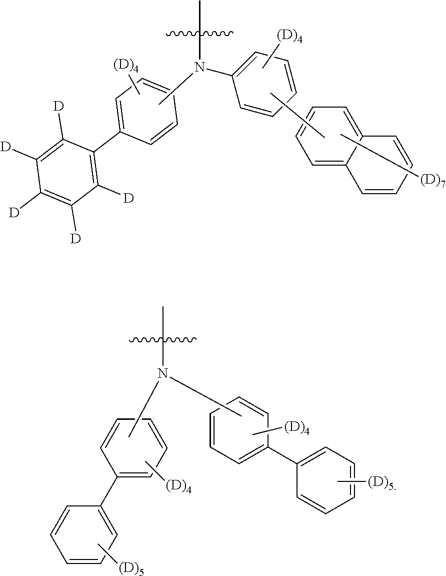

10. The aromatic amine compound according to claim 1, wherein the aromatic amine compound is selected from the group consisting of the following compounds:

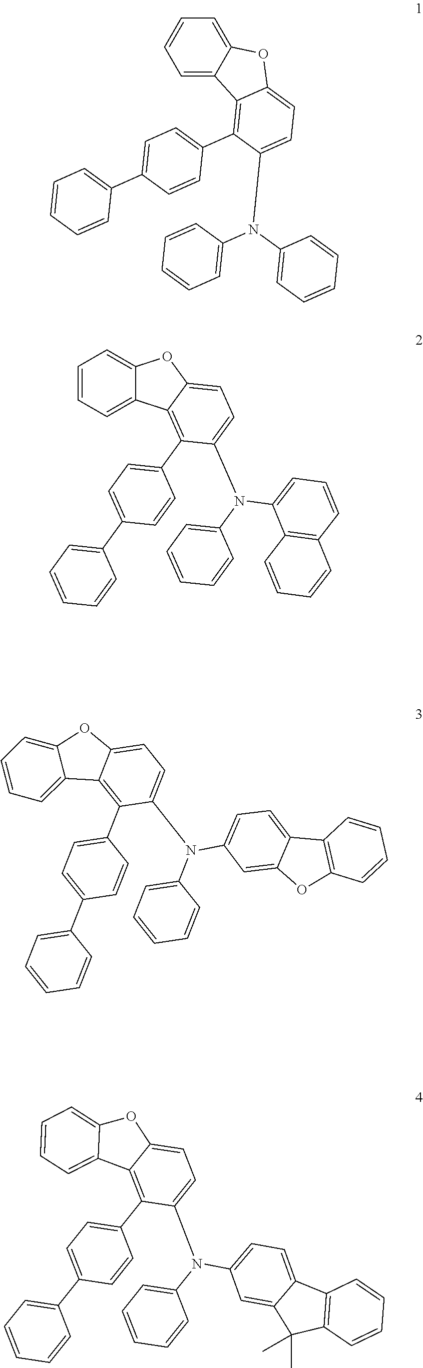 [SMILES: c1ccc(-c2ccc(-c3c(N(c4ccccc4)c4ccccc4)ccc4oc5ccccc5c34)cc2)cc1 ; c1ccc(-c2ccc(-c3c(N(c4ccccc4)c4cccc5ccccc45)ccc4oc5ccccc5c34)cc2)cc1 ; c1ccc(-c2ccc(-c3c(N(c4ccccc4)c4ccc5c(c4)oc4ccccc45)ccc4oc5ccccc5c34)cc2)cc1 ; CC1(C)c2ccccc2-c2ccc(N(c3ccccc3)c3ccc4oc5ccccc5c4c3-c3ccc(-c4ccccc4)cc3)cc21]

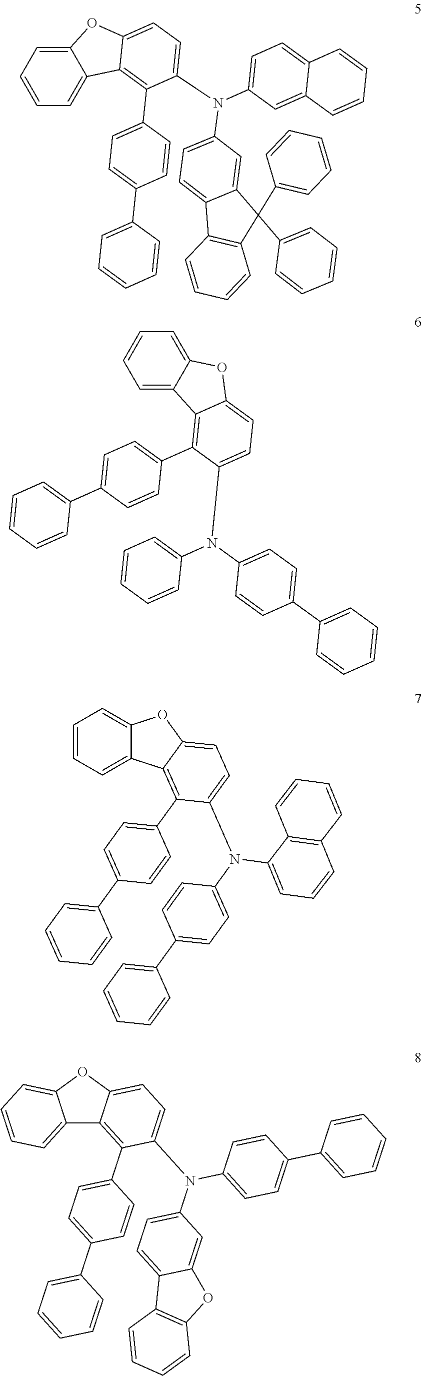

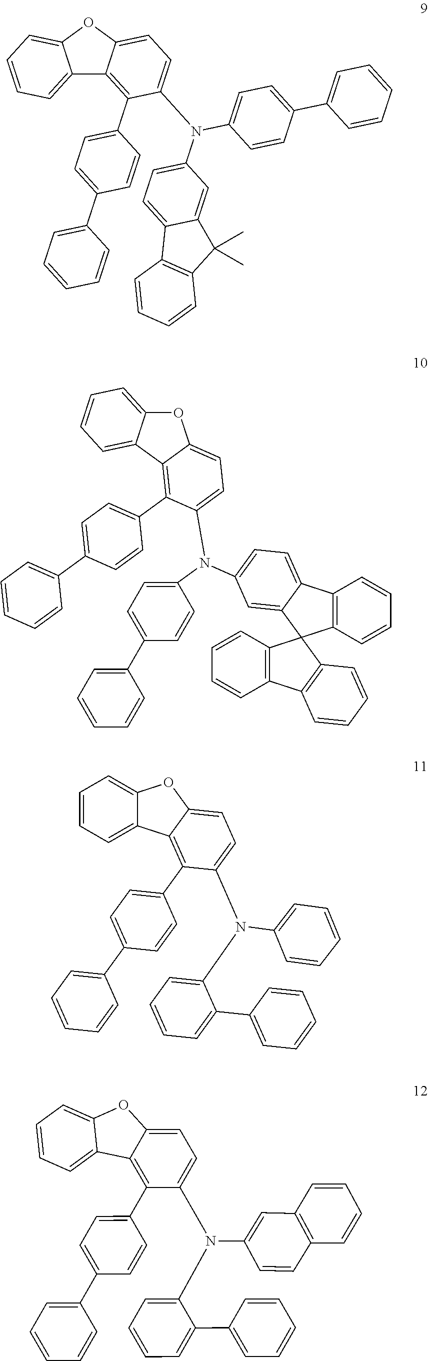 [SMILES: CC1(C)c2ccccc2-c2ccc(N(c3ccc(-c4ccccc4)cc3)c3ccc4oc5ccccc5c4c3-c3ccc(-c4ccccc4)cc3)cc21 ; c1ccc(-c2ccc(-c3c(N(c4ccc(-c5ccccc5)cc4)c4ccc5c(c4)C4(c6ccccc6-c6ccccc64)c4ccccc4-5)ccc4oc5ccccc5c34)cc2)cc1 ; c1ccc(-c2ccc(-c3c(N(c4ccccc4)c4ccccc4-c4ccccc4)ccc4oc5ccccc5c34)cc2)cc1 ; c1ccc(-c2ccc(-c3c(N(c4ccc5ccccc5c4)c4ccccc4-c4ccccc4)ccc4oc5ccccc5c34)cc2)cc1]

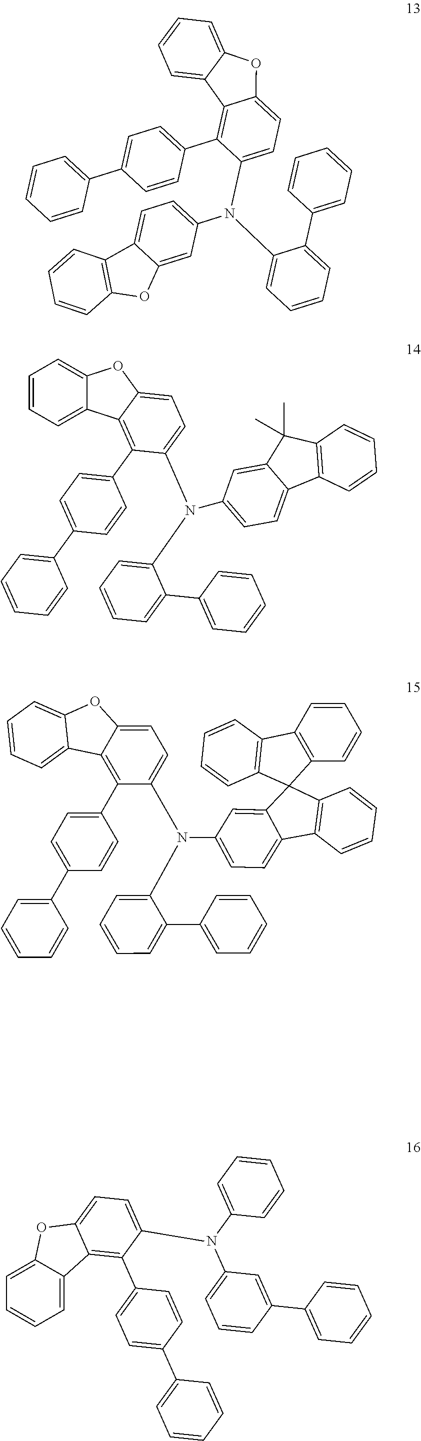 [SMILES: c1ccc(-c2ccc(-c3c(N(c4ccc5c(c4)oc4ccccc45)c4ccccc4-c4ccccc4)ccc4oc5ccccc5c34)cc2)cc1 ; CC1(C)c2ccccc2-c2ccc(N(c3ccccc3-c3ccccc3)c3ccc4oc5ccccc5c4c3-c3ccc(-c4ccccc4)cc3)cc21 ; c1ccc(-c2ccc(-c3c(N(c4ccc5c(c4)C4(c6ccccc6-c6ccccc64)c4ccccc4-5)c4ccccc4-c4ccccc4)ccc4oc5ccccc5c34)cc2)cc1 ; c1ccc(-c2ccc(-c3c(N(c4ccccc4)c4cccc(-c5ccccc5)c4)ccc4oc5ccccc5c34)cc2)cc1]

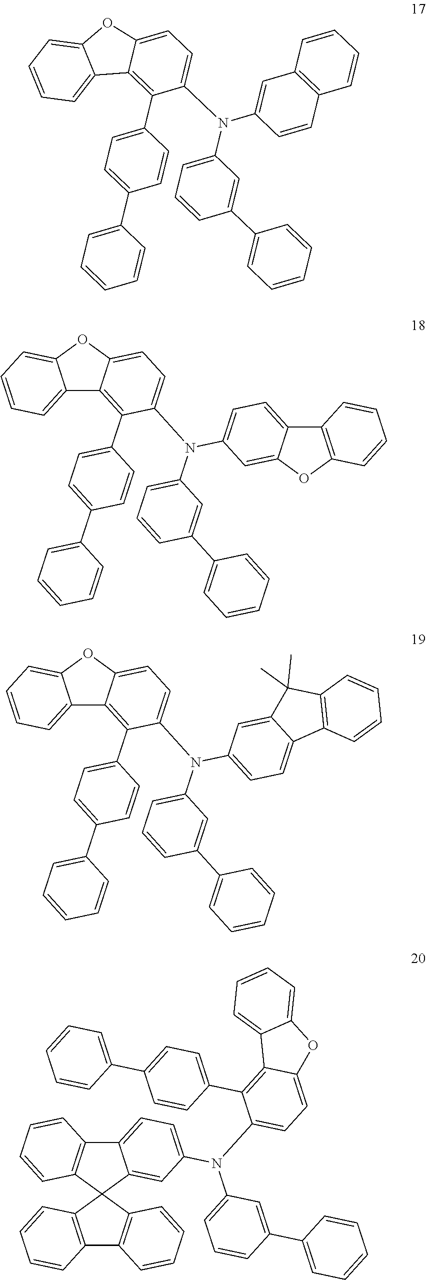

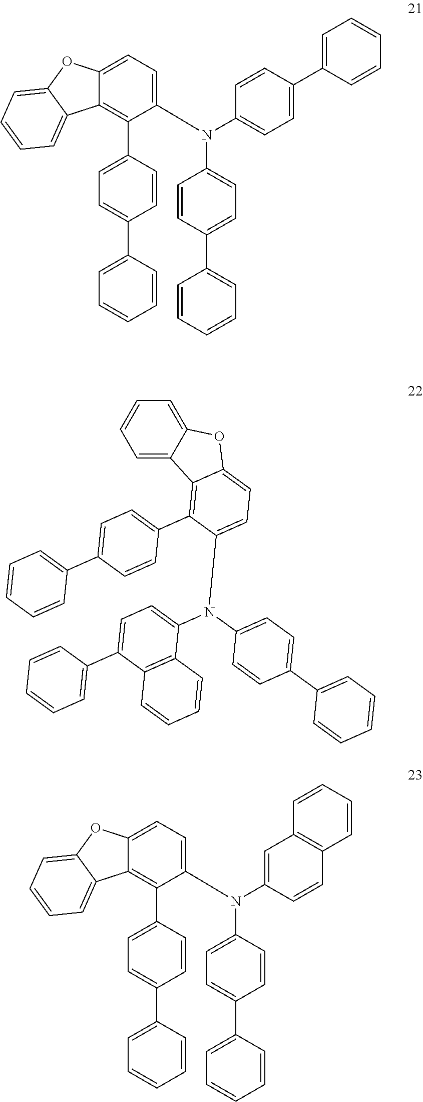

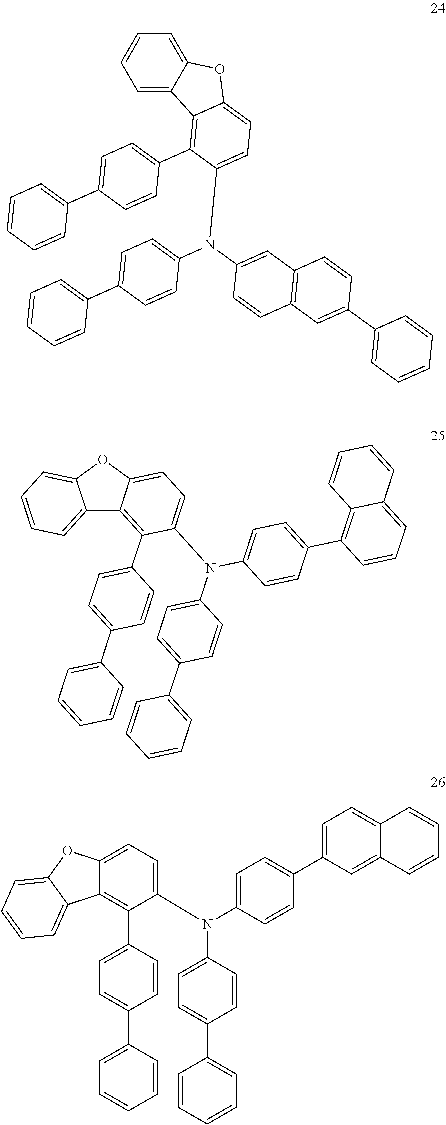 [SMILES: c1ccc(-c2ccc(-c3c(N(c4ccc(-c5ccccc5)cc4)c4ccc5cc(-c6ccccc6)ccc5c4)ccc4oc5ccccc5c34)cc2)cc1 ; c1ccc(-c2ccc(-c3c(N(c4ccc(-c5ccccc5)cc4)c4ccc(-c5cccc6ccccc56)cc4)ccc4oc5ccccc5c34)cc2)cc1 ; c1ccc(-c2ccc(-c3c(N(c4ccc(-c5ccccc5)cc4)c4ccc(-c5ccc6ccccc6c5)cc4)ccc4oc5ccccc5c34)cc2)cc1]

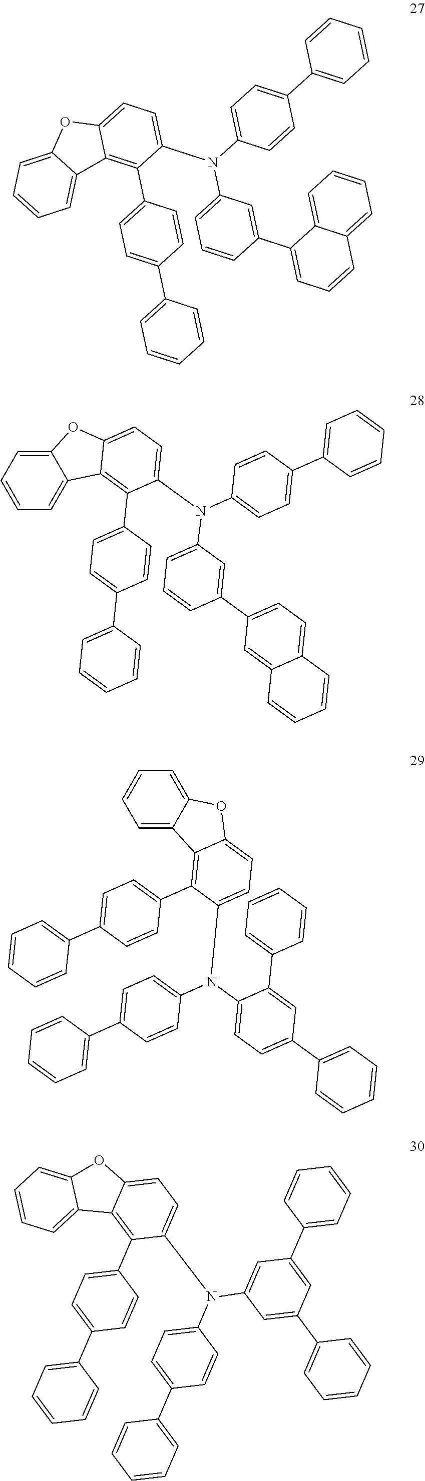 [SMILES: c1ccc(-c2ccc(-c3c(N(c4ccc(-c5ccccc5)cc4)c4cccc(-c5cccc6ccccc56)c4)ccc4oc5ccccc5c34)cc2)cc1 ; c1ccc(-c2ccc(-c3c(N(c4ccc(-c5ccccc5)cc4)c4cccc(-c5ccc6ccccc6c5)c4)ccc4oc5ccccc5c34)cc2)cc1 ; c1ccc(-c2ccc(-c3c(N(c4ccc(-c5ccccc5)cc4)c4ccc(-c5ccccc5)cc4-c4ccccc4)ccc4oc5ccccc5c34)cc2)cc1 ; c1ccc(-c2ccc(-c3c(N(c4ccc(-c5ccccc5)cc4)c4cc(-c5ccccc5)cc(-c5ccccc5)c4)ccc4oc5ccccc5c34)cc2)cc1]

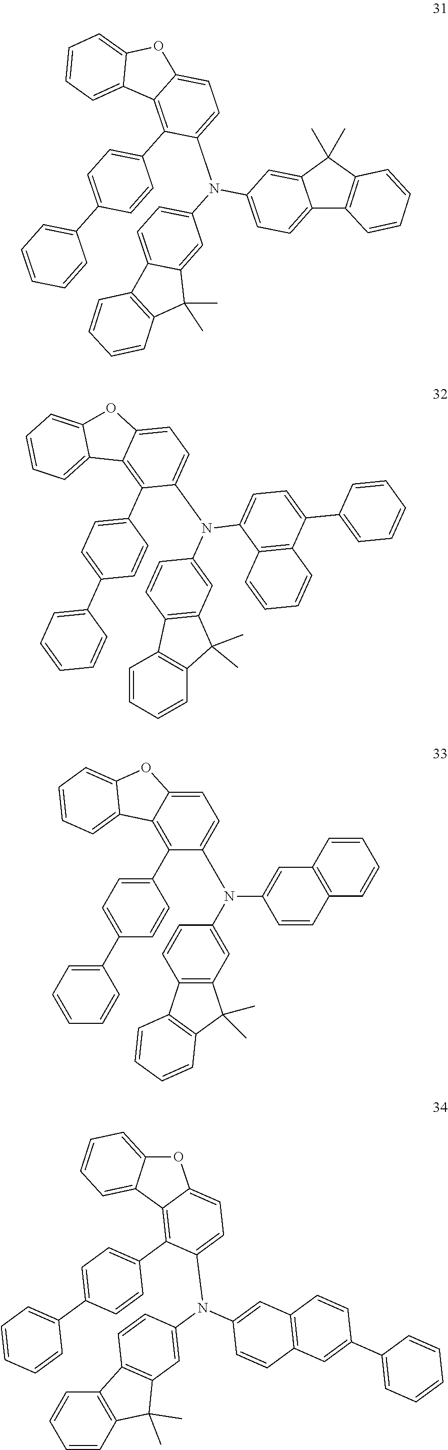 [SMILES: CC1(C)c2ccccc2-c2ccc(N(c3ccc4c(c3)C(C)(C)c3ccccc3-4)c3ccc4oc5ccccc5c4c3-c3ccc(-c4ccccc4)cc3)cc21 ; CC1(C)c2ccccc2-c2ccc(N(c3ccc4oc5ccccc5c4c3-c3ccc(-c4ccccc4)cc3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc(-c4ccccc4)cc3)cc21 ; CC1(C)c2ccccc2-c2ccc(N(c3ccc4cc(-c5ccccc5)ccc4c3)c3ccc4oc5ccccc5c4c3-c3ccc(-c4ccccc4)cc3)cc21]

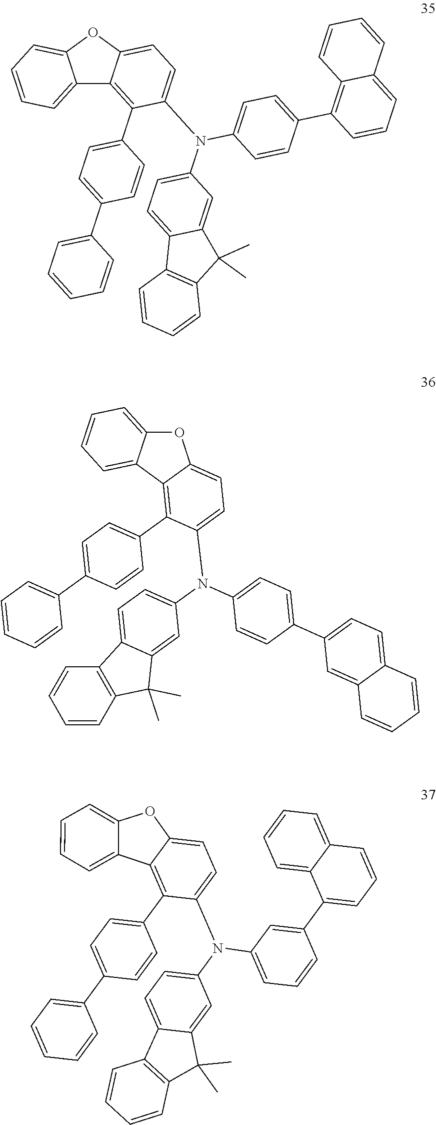 [SMILES: CC1(C)c2ccccc2-c2ccc(N(c3ccc(-c4cccc5ccccc45)cc3)c3ccc4oc5ccccc5c4c3-c3ccc(-c4ccccc4)cc3)cc21 ; CC1(C)c2ccccc2-c2ccc(N(c3ccc(-c4ccc5ccccc5c4)cc3)c3ccc4oc5ccccc5c4c3-c3ccc(-c4ccccc4)cc3)cc21 ; CC1(C)c2ccccc2-c2ccc(N(c3cccc(-c4cccc5ccccc45)c3)c3ccc4oc5ccccc5c4c3-c3ccc(-c4ccccc4)cc3)cc21]

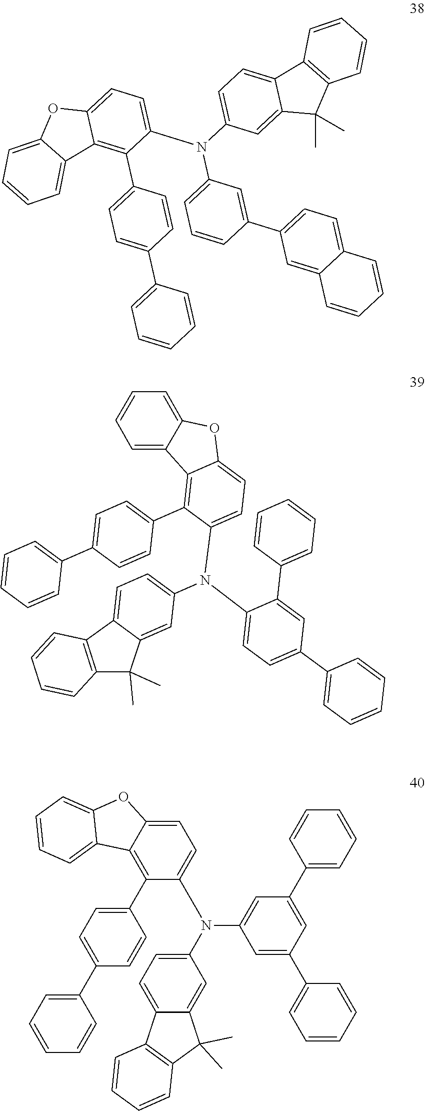

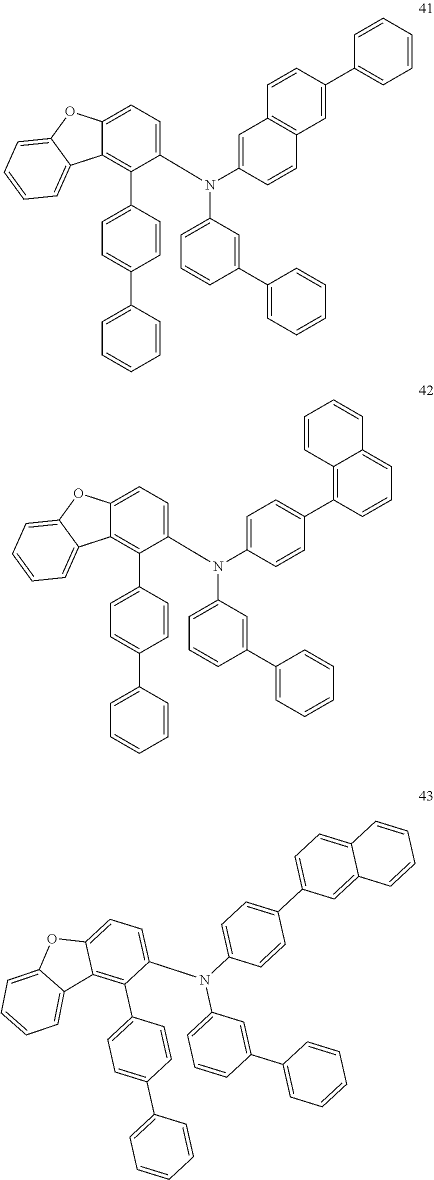 [SMILES: c1ccc(-c2ccc(-c3c(N(c4cccc(-c5ccccc5)c4)c4ccc5cc(-c6ccccc6)ccc5c4)ccc4oc5ccccc5c34)cc2)cc1 ; c1ccc(-c2ccc(-c3c(N(c4ccc(-c5cccc6ccccc56)cc4)c4cccc(-c5ccccc5)c4)ccc4oc5ccccc5c34)cc2)cc1 ; c1ccc(-c2ccc(-c3c(N(c4ccc(-c5ccc6ccccc6c5)cc4)c4cccc(-c5ccccc5)c4)ccc4oc5ccccc5c34)cc2)cc1]

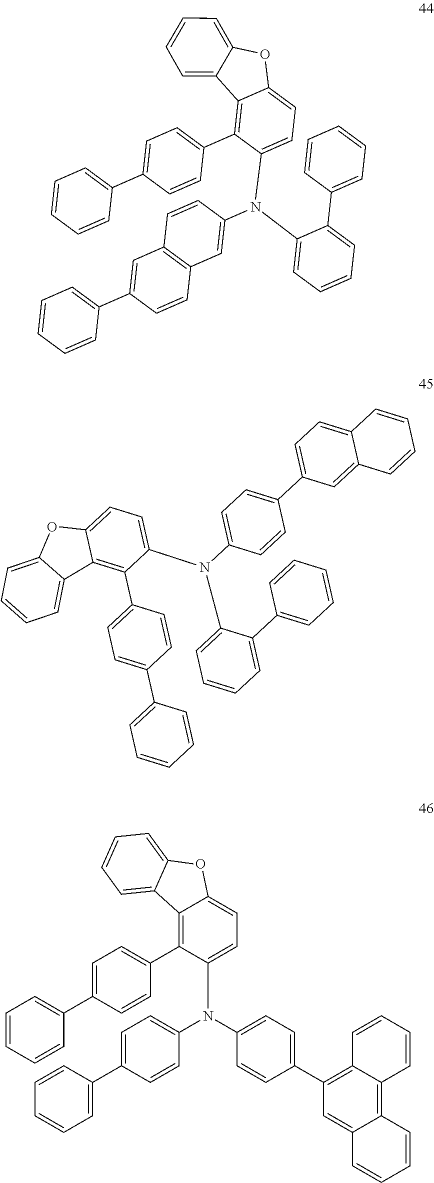 [SMILES: c1ccc(-c2ccc(-c3c(N(c4ccc5cc(-c6ccccc6)ccc5c4)c4ccccc4-c4ccccc4)ccc4oc5ccccc5c34)cc2)cc1 ; c1ccc(-c2ccc(-c3c(N(c4ccc(-c5ccc6ccccc6c5)cc4)c4ccccc4-c4ccccc4)ccc4oc5ccccc5c34)cc2)cc1 ; c1ccc(-c2ccc(-c3c(N(c4ccc(-c5ccccc5)cc4)c4ccc(-c5cc6ccccc6c6ccccc56)cc4)ccc4oc5ccccc5c34)cc2)cc1]

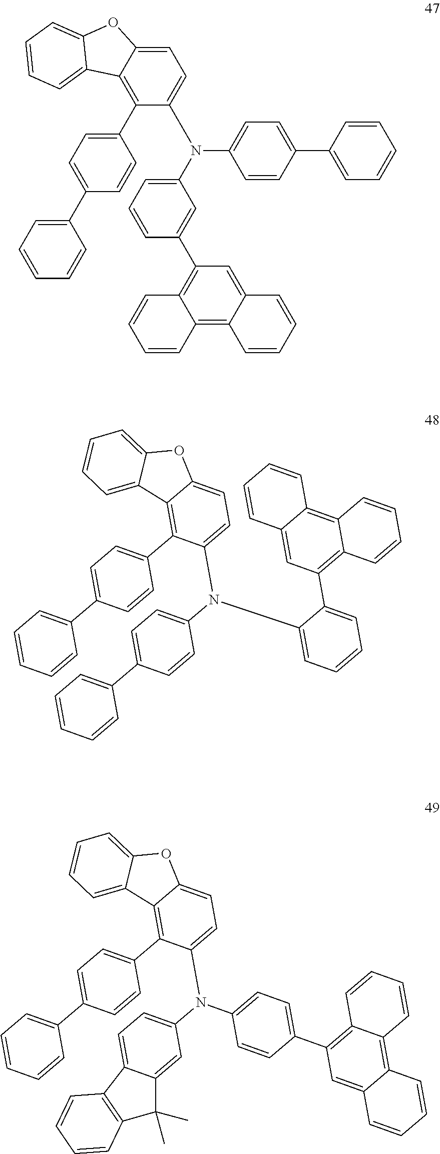 [SMILES: c1ccc(-c2ccc(-c3c(N(c4ccc(-c5ccccc5)cc4)c4cccc(-c5cc6ccccc6c6ccccc56)c4)ccc4oc5ccccc5c34)cc2)cc1 ; c1ccc(-c2ccc(-c3c(N(c4ccc(-c5ccccc5)cc4)c4ccccc4-c4cc5ccccc5c5ccccc45)ccc4oc5ccccc5c34)cc2)cc1 ; CC1(C)c2ccccc2-c2ccc(N(c3ccc(-c4cc5ccccc5c5ccccc45)cc3)c3ccc4oc5ccccc5c4c3-c3ccc(-c4ccccc4)cc3)cc21]

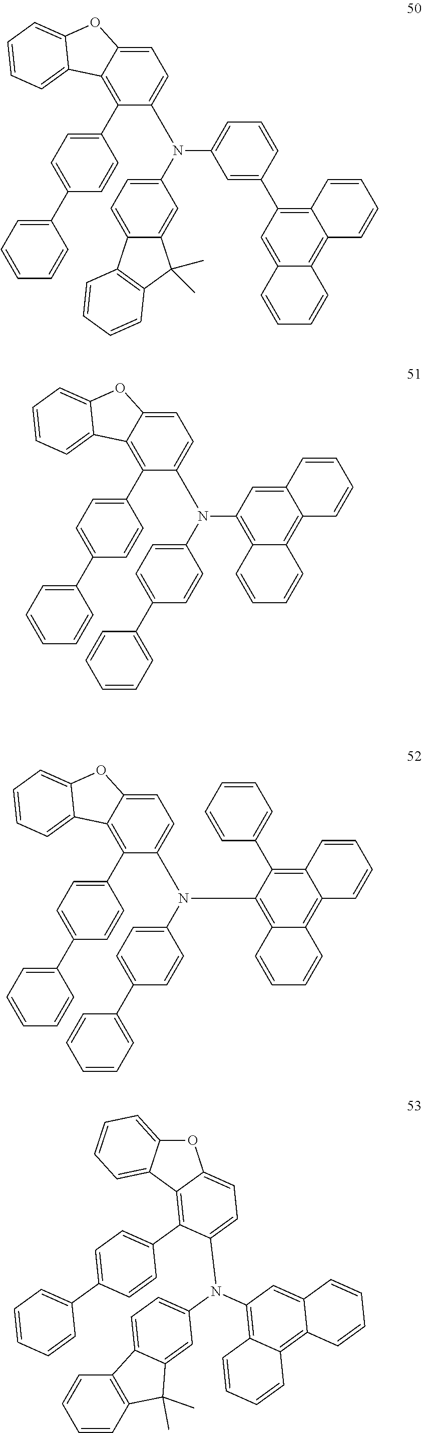 [SMILES: CC1(C)c2ccccc2-c2ccc(N(c3cccc(-c4cc5ccccc5c5ccccc45)c3)c3ccc4oc5ccccc5c4c3-c3ccc(-c4ccccc4)cc3)cc21 ; c1ccc(-c2ccc(-c3c(N(c4ccc(-c5ccccc5)cc4)c4cc5ccccc5c5ccccc45)ccc4oc5ccccc5c34)cc2)cc1 ; c1ccc(-c2ccc(-c3c(N(c4ccc(-c5ccccc5)cc4)c4c(-c5ccccc5)c5ccccc5c5ccccc45)ccc4oc5ccccc5c34)cc2)cc1 ; CC1(C)c2ccccc2-c2ccc(N(c3ccc4oc5ccccc5c4c3-c3ccc(-c4ccccc4)cc3)c3cc4ccccc4c4ccccc34)cc21]

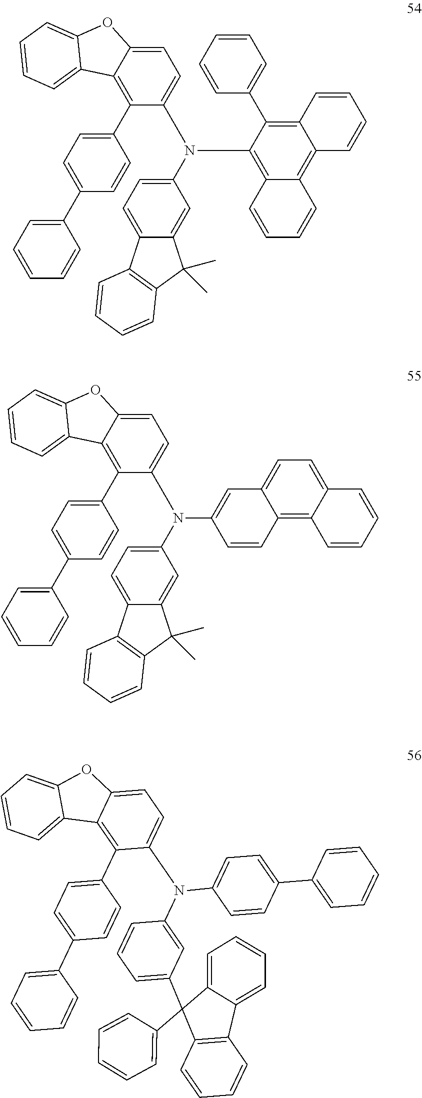 [SMILES: CC1(C)c2ccccc2-c2ccc(N(c3ccc4oc5ccccc5c4c3-c3ccc(-c4ccccc4)cc3)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3ccc(-c4ccccc4)cc3)cc21 ; c1ccc(-c2ccc(-c3c(N(c4ccc(-c5ccccc5)cc4)c4cccc(C5(c6ccccc6)c6ccccc6-c6ccccc65)c4)ccc4oc5ccccc5c34)cc2)cc1]

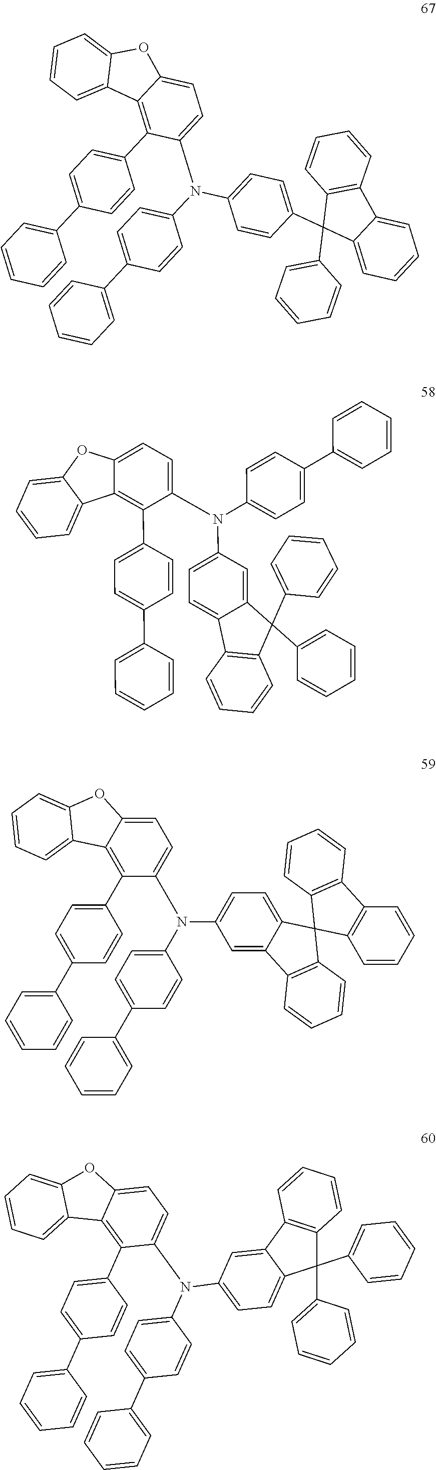 [SMILES: c1ccc(-c2ccc(-c3c(N(c4ccc(-c5ccccc5)cc4)c4ccc(C5(c6ccccc6)c6ccccc6-c6ccccc65)cc4)ccc4oc5ccccc5c34)cc2)cc1 ; c1ccc(-c2ccc(-c3c(N(c4ccc(-c5ccccc5)cc4)c4ccc5c(c4)C(c4ccccc4)(c4ccccc4)c4ccccc4-5)ccc4oc5ccccc5c34)cc2)cc1 ; c1ccc(-c2ccc(-c3c(N(c4ccc(-c5ccccc5)cc4)c4ccc5c(c4)-c4ccccc4C54c5ccccc5-c5ccccc54)ccc4oc5ccccc5c34)cc2)cc1 ; c1ccc(-c2ccc(-c3c(N(c4ccc(-c5ccccc5)cc4)c4ccc5c(c4)-c4ccccc4C5(c4ccccc4)c4ccccc4)ccc4oc5ccccc5c34)cc2)cc1]

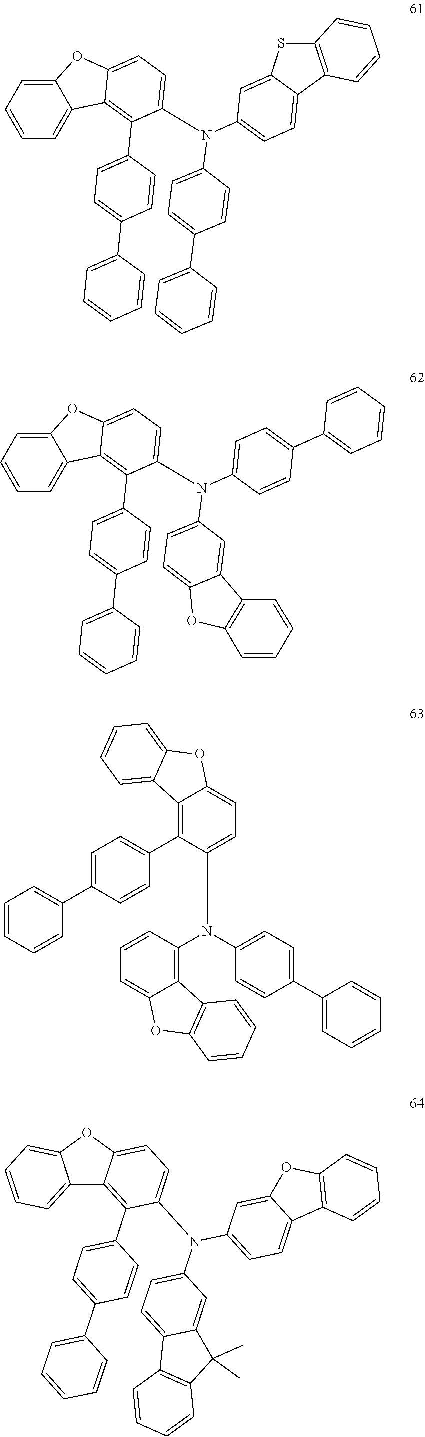 [SMILES: c1ccc(-c2ccc(-c3c(N(c4ccc(-c5ccccc5)cc4)c4ccc5c(c4)sc4ccccc45)ccc4oc5ccccc5c34)cc2)cc1 ; c1ccc(-c2ccc(-c3c(N(c4ccc(-c5ccccc5)cc4)c4ccc5oc6ccccc6c5c4)ccc4oc5ccccc5c34)cc2)cc1 ; c1ccc(-c2ccc(-c3c(N(c4ccc(-c5ccccc5)cc4)c4cccc5oc6ccccc6c45)ccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc(-c4ccccc4)cc3)cc21]

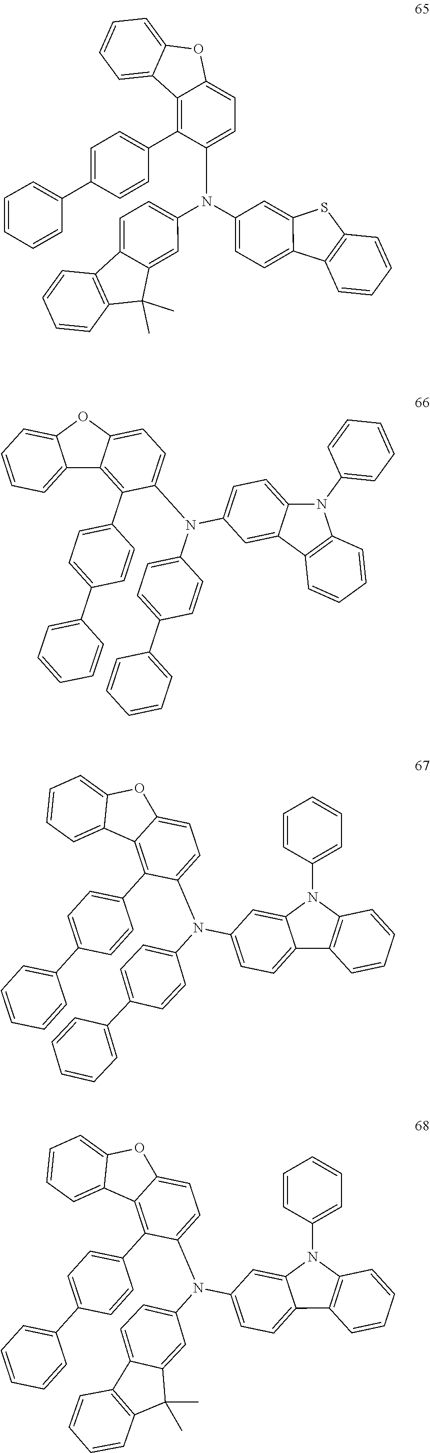 [SMILES: CC1(C)c2ccccc2-c2ccc(N(c3ccc4c(c3)sc3ccccc34)c3ccc4oc5ccccc5c4c3-c3ccc(-c4ccccc4)cc3)cc21 ; c1ccc(-c2ccc(-c3c(N(c4ccc(-c5ccccc5)cc4)c4ccc5c(c4)c4ccccc4n5-c4ccccc4)ccc4oc5ccccc5c34)cc2)cc1 ; c1ccc(-c2ccc(-c3c(N(c4ccc(-c5ccccc5)cc4)c4ccc5c6ccccc6n(-c6ccccc6)c5c4)ccc4oc5ccccc5c34)cc2)cc1 ; CC1(C)c2ccccc2-c2ccc(N(c3ccc4c5ccccc5n(-c5ccccc5)c4c3)c3ccc4oc5ccccc5c4c3-c3ccc(-c4ccccc4)cc3)cc21]

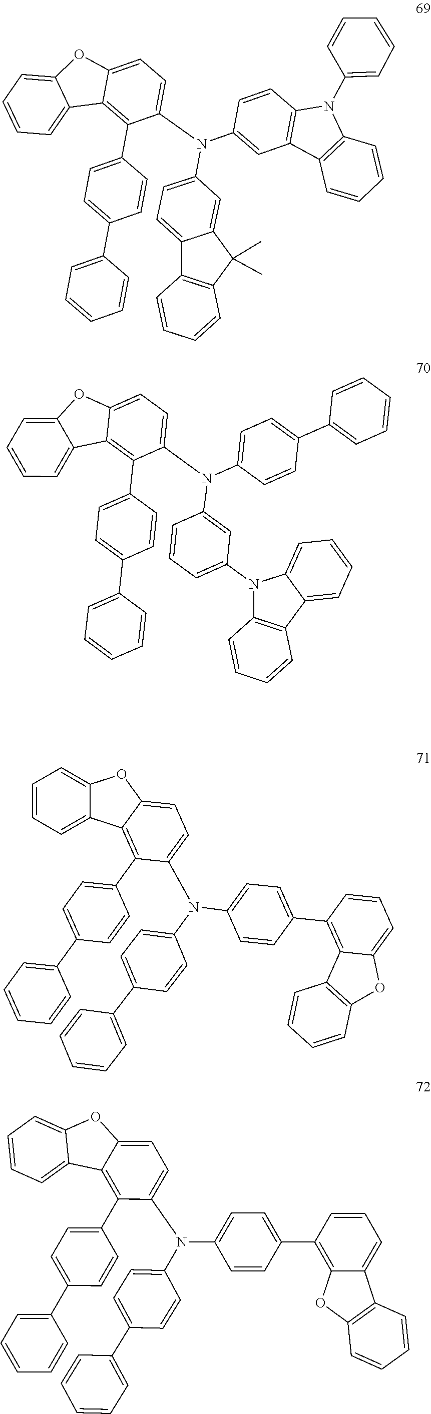

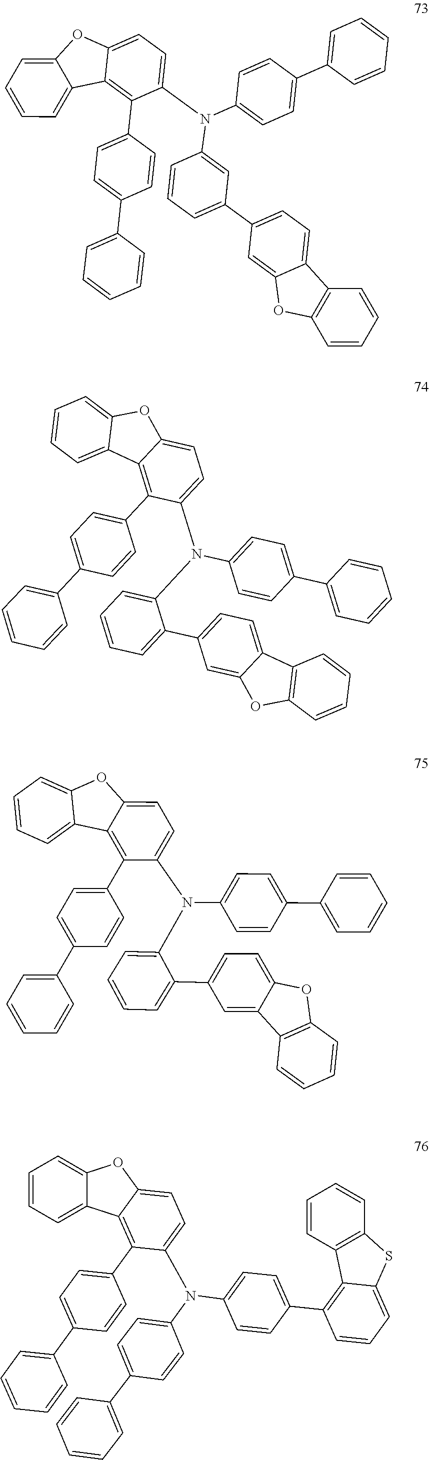 [SMILES: c1ccc(-c2ccc(-c3c(N(c4ccc(-c5ccccc5)cc4)c4cccc(-c5ccc6c(c5)oc5ccccc56)c4)ccc4oc5ccccc5c34)cc2)cc1 ; c1ccc(-c2ccc(-c3c(N(c4ccc(-c5ccccc5)cc4)c4ccccc4-c4ccc5c(c4)oc4ccccc45)ccc4oc5ccccc5c34)cc2)cc1 ; c1ccc(-c2ccc(-c3c(N(c4ccc(-c5ccccc5)cc4)c4ccccc4-c4ccc5oc6ccccc6c5c4)ccc4oc5ccccc5c34)cc2)cc1 ; c1ccc(-c2ccc(-c3c(N(c4ccc(-c5ccccc5)cc4)c4ccc(-c5cccc6sc7ccccc7c56)cc4)ccc4oc5ccccc5c34)cc2)cc1]

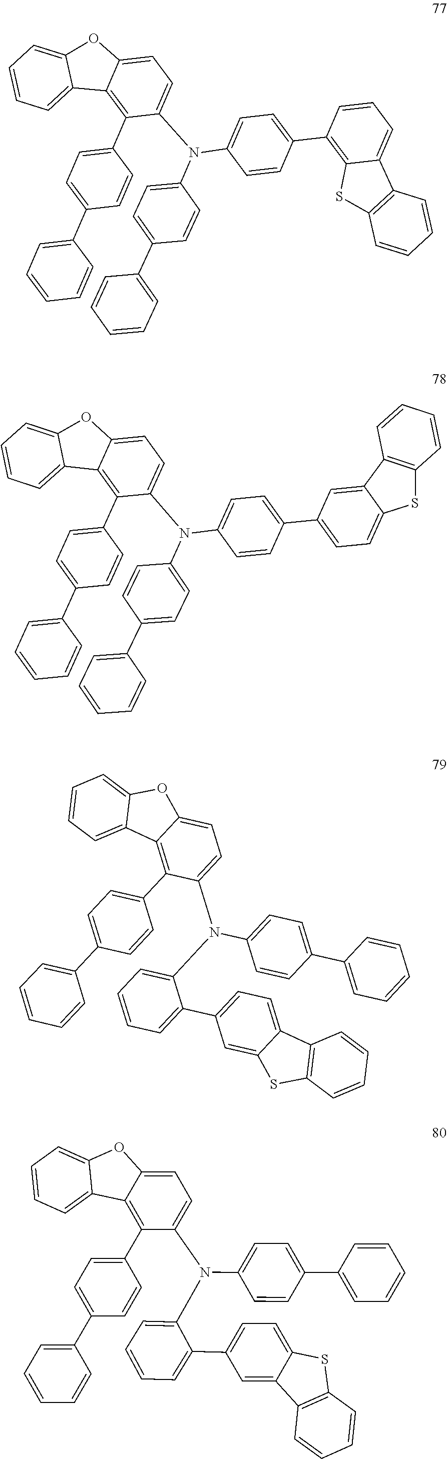 [SMILES: c1ccc(-c2ccc(-c3c(N(c4ccc(-c5ccccc5)cc4)c4ccc(-c5cccc6c5sc5ccccc56)cc4)ccc4oc5ccccc5c34)cc2)cc1 ; c1ccc(-c2ccc(-c3c(N(c4ccc(-c5ccccc5)cc4)c4ccc(-c5ccc6sc7ccccc7c6c5)cc4)ccc4oc5ccccc5c34)cc2)cc1 ; c1ccc(-c2ccc(-c3c(N(c4ccc(-c5ccccc5)cc4)c4ccccc4-c4ccc5c(c4)sc4ccccc45)ccc4oc5ccccc5c34)cc2)cc1 ; c1ccc(-c2ccc(-c3c(N(c4ccc(-c5ccccc5)cc4)c4ccccc4-c4ccc5sc6ccccc6c5c4)ccc4oc5ccccc5c34)cc2)cc1]

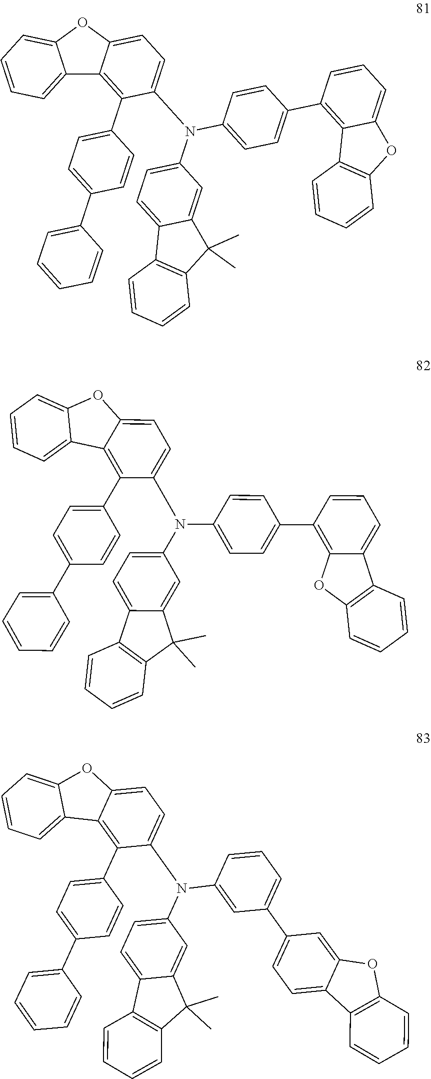 [SMILES: CC1(C)c2ccccc2-c2ccc(N(c3ccc(-c4cccc5oc6ccccc6c45)cc3)c3ccc4oc5ccccc5c4c3-c3ccc(-c4ccccc4)cc3)cc21 ; CC1(C)c2ccccc2-c2ccc(N(c3ccc(-c4cccc5c4oc4ccccc45)cc3)c3ccc4oc5ccccc5c4c3-c3ccc(-c4ccccc4)cc3)cc21 ; CC1(C)c2ccccc2-c2ccc(N(c3cccc(-c4ccc5c(c4)oc4ccccc45)c3)c3ccc4oc5ccccc5c4c3-c3ccc(-c4ccccc4)cc3)cc21]

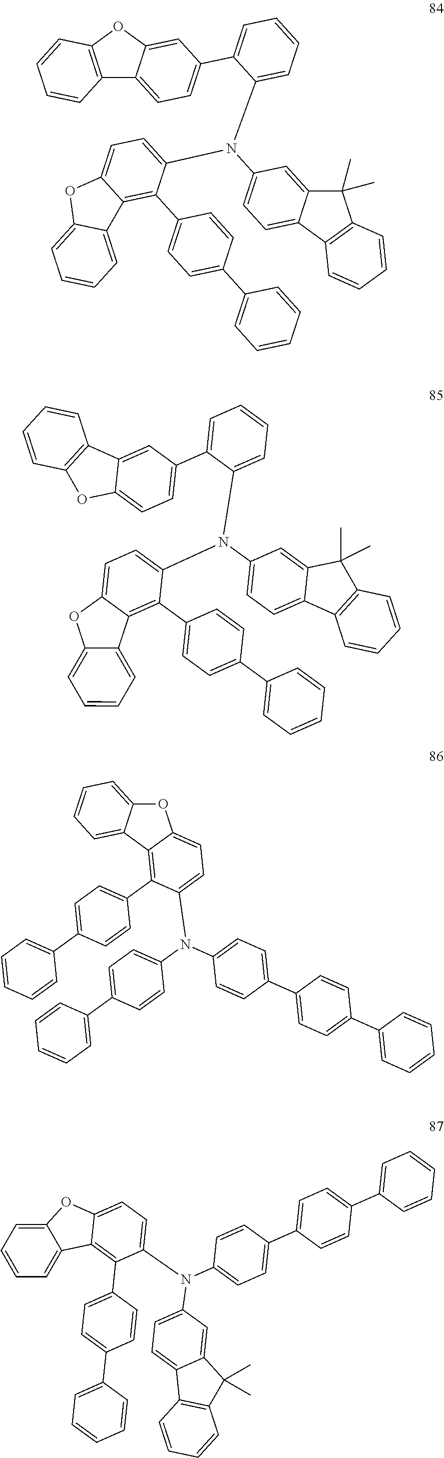 [SMILES: CC1(C)c2ccccc2-c2ccc(N(c3ccccc3-c3ccc4c(c3)oc3ccccc34)c3ccc4oc5ccccc5c4c3-c3ccc(-c4ccccc4)cc3)cc21 ; CC1(C)c2ccccc2-c2ccc(N(c3ccccc3-c3ccc4oc5ccccc5c4c3)c3ccc4oc5ccccc5c4c3-c3ccc(-c4ccccc4)cc3)cc21 ; c1ccc(-c2ccc(-c3ccc(N(c4ccc(-c5ccccc5)cc4)c4ccc5oc6ccccc6c5c4-c4ccc(-c5ccccc5)cc4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc(-c4ccccc4)cc3)cc21]

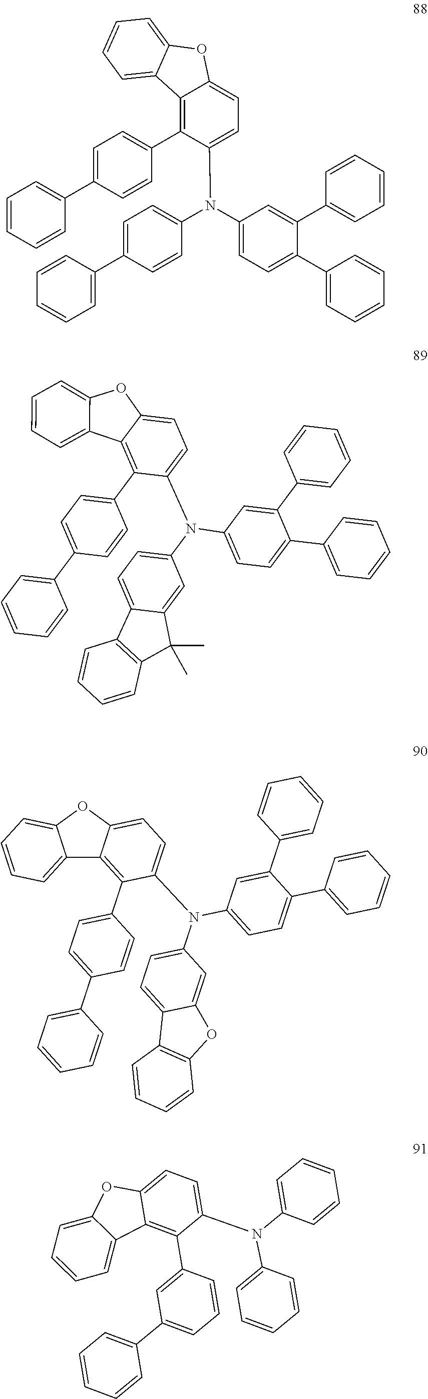 [SMILES: c1ccc(-c2ccc(-c3c(N(c4ccc(-c5ccccc5)cc4)c4ccc(-c5ccccc5)c(-c5ccccc5)c4)ccc4oc5ccccc5c34)cc2)cc1 ; CC1(C)c2ccccc2-c2ccc(N(c3ccc(-c4ccccc4)c(-c4ccccc4)c3)c3ccc4oc5ccccc5c4c3-c3ccc(-c4ccccc4)cc3)cc21 ; c1ccc(-c2ccc(-c3c(N(c4ccc(-c5ccccc5)c(-c5ccccc5)c4)c4ccc5c(c4)oc4ccccc45)ccc4oc5ccccc5c34)cc2)cc1 ; c1ccc(-c2cccc(-c3c(N(c4ccccc4)c4ccccc4)ccc4oc5ccccc5c34)c2)cc1]

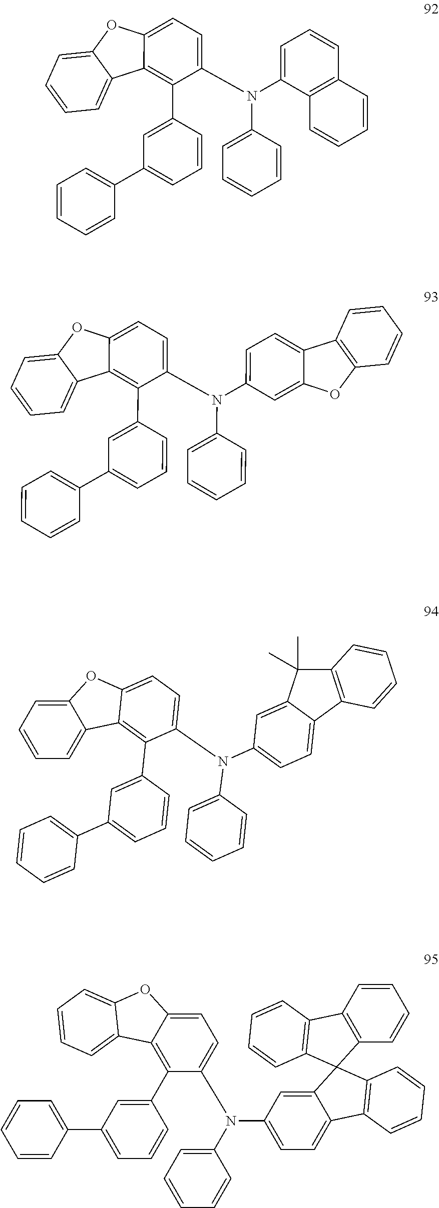 [SMILES: c1ccc(-c2cccc(-c3c(N(c4ccccc4)c4cccc5ccccc45)ccc4oc5ccccc5c34)c2)cc1 ; c1ccc(-c2cccc(-c3c(N(c4ccccc4)c4ccc5c(c4)oc4ccccc45)ccc4oc5ccccc5c34)c2)cc1 ; CC1(C)c2ccccc2-c2ccc(N(c3ccccc3)c3ccc4oc5ccccc5c4c3-c3cccc(-c4ccccc4)c3)cc21 ; c1ccc(-c2cccc(-c3c(N(c4ccccc4)c4ccc5c(c4)C4(c6ccccc6-c6ccccc64)c4ccccc4-5)ccc4oc5ccccc5c34)c2)cc1]

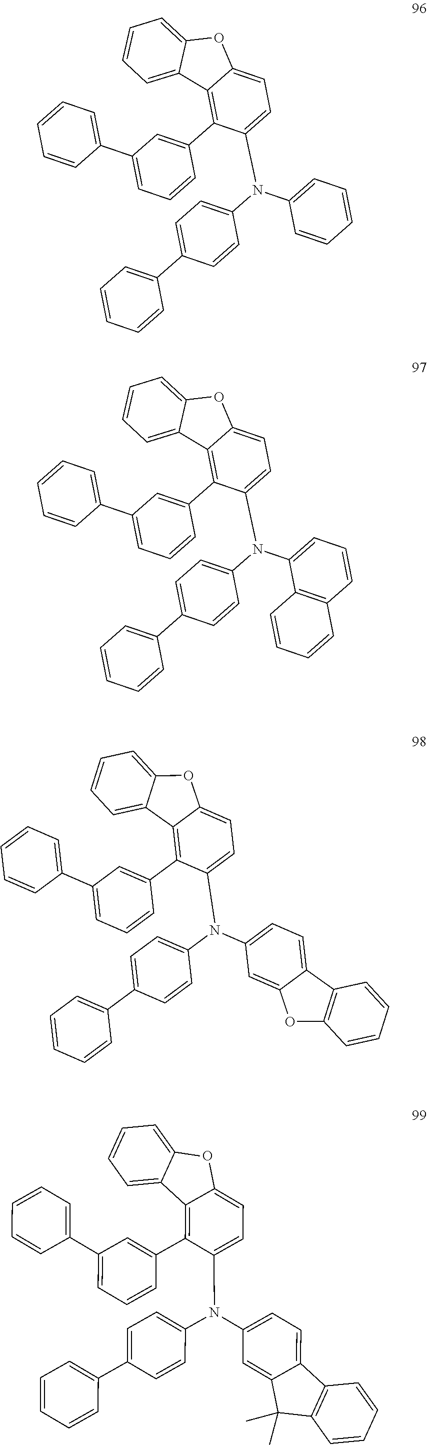 [SMILES: c1ccc(-c2ccc(N(c3ccccc3)c3ccc4oc5ccccc5c4c3-c3cccc(-c4ccccc4)c3)cc2)cc1 ; c1ccc(-c2ccc(N(c3ccc4oc5ccccc5c4c3-c3cccc(-c4ccccc4)c3)c3cccc4ccccc34)cc2)cc1 ; c1ccc(-c2ccc(N(c3ccc4c(c3)oc3ccccc34)c3ccc4oc5ccccc5c4c3-c3cccc(-c4ccccc4)c3)cc2)cc1 ; CC1(C)c2ccccc2-c2ccc(N(c3ccc(-c4ccccc4)cc3)c3ccc4oc5ccccc5c4c3-c3cccc(-c4ccccc4)c3)cc21]

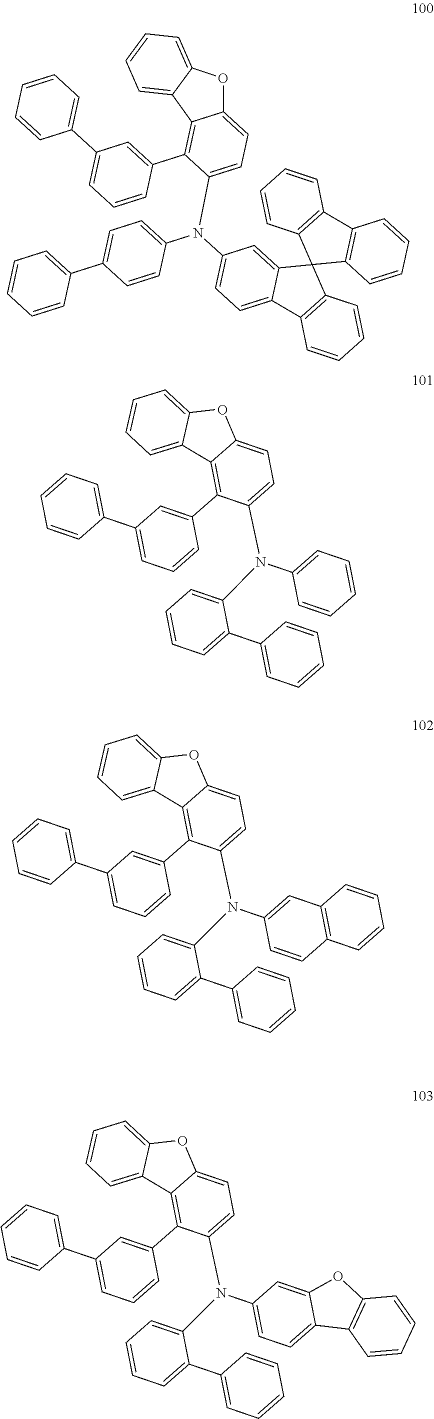 [SMILES: c1ccc(-c2ccc(N(c3ccc4c(c3)C3(c5ccccc5-c5ccccc53)c3ccccc3-4)c3ccc4oc5ccccc5c4c3-c3cccc(-c4ccccc4)c3)cc2)cc1 ; c1ccc(-c2cccc(-c3c(N(c4ccccc4)c4ccccc4-c4ccccc4)ccc4oc5ccccc5c34)c2)cc1 ; c1ccc(-c2cccc(-c3c(N(c4ccc5ccccc5c4)c4ccccc4-c4ccccc4)ccc4oc5ccccc5c34)c2)cc1 ; c1ccc(-c2cccc(-c3c(N(c4ccc5c(c4)oc4ccccc45)c4ccccc4-c4ccccc4)ccc4oc5ccccc5c34)c2)cc1]

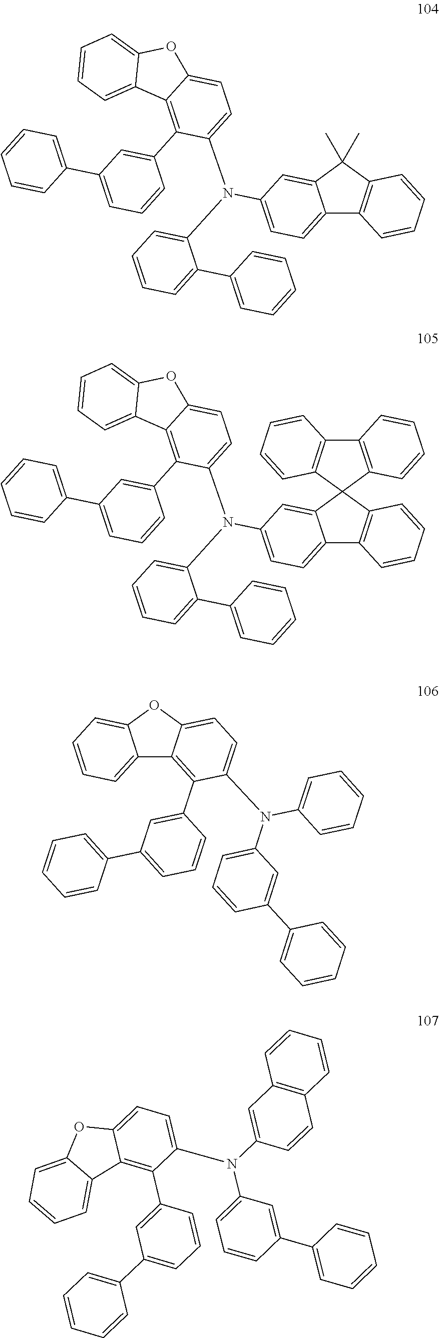

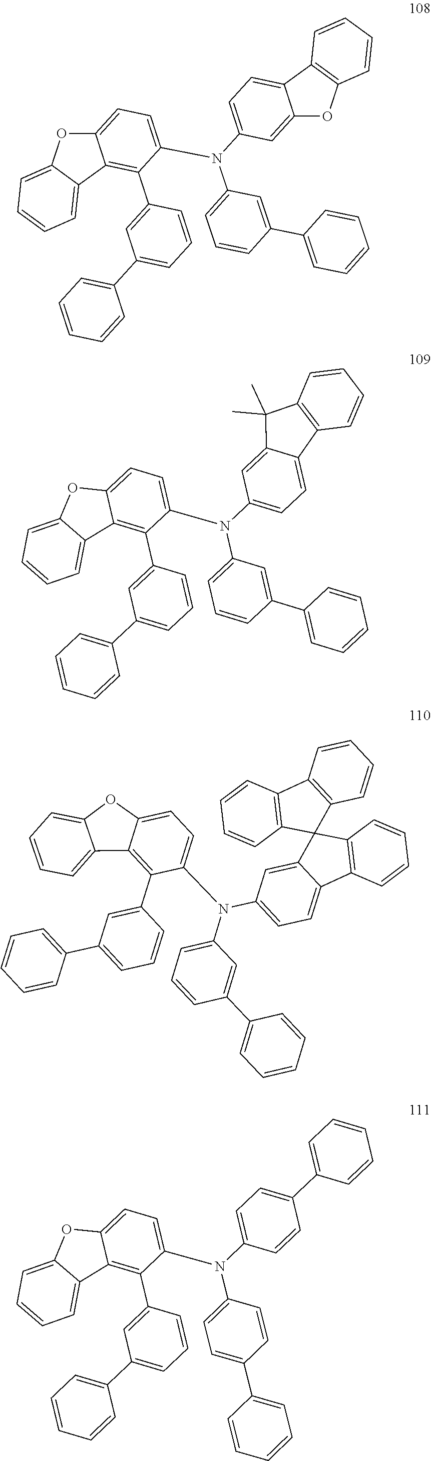 [SMILES: c1ccc(-c2cccc(-c3c(N(c4cccc(-c5ccccc5)c4)c4ccc5c(c4)oc4ccccc45)ccc4oc5ccccc5c34)c2)cc1 ; CC1(C)c2ccccc2-c2ccc(N(c3cccc(-c4ccccc4)c3)c3ccc4oc5ccccc5c4c3-c3cccc(-c4ccccc4)c3)cc21 ; c1ccc(-c2cccc(-c3c(N(c4cccc(-c5ccccc5)c4)c4ccc5c(c4)C4(c6ccccc6-c6ccccc64)c4ccccc4-5)ccc4oc5ccccc5c34)c2)cc1 ; c1ccc(-c2ccc(N(c3ccc(-c4ccccc4)cc3)c3ccc4oc5ccccc5c4c3-c3cccc(-c4ccccc4)c3)cc2)cc1]

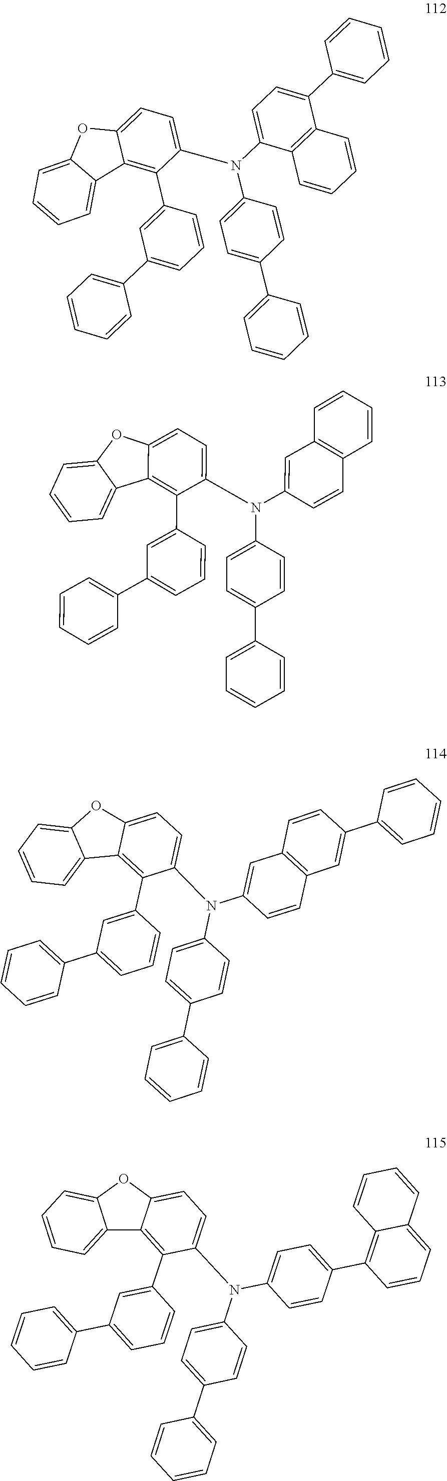 [SMILES: c1ccc(-c2ccc(N(c3ccc4oc5ccccc5c4c3-c3cccc(-c4ccccc4)c3)c3ccc(-c4ccccc4)c4ccccc34)cc2)cc1 ; c1ccc(-c2ccc(N(c3ccc4ccccc4c3)c3ccc4oc5ccccc5c4c3-c3cccc(-c4ccccc4)c3)cc2)cc1 ; c1ccc(-c2ccc(N(c3ccc4cc(-c5ccccc5)ccc4c3)c3ccc4oc5ccccc5c4c3-c3cccc(-c4ccccc4)c3)cc2)cc1 ; c1ccc(-c2ccc(N(c3ccc(-c4cccc5ccccc45)cc3)c3ccc4oc5ccccc5c4c3-c3cccc(-c4ccccc4)c3)cc2)cc1]

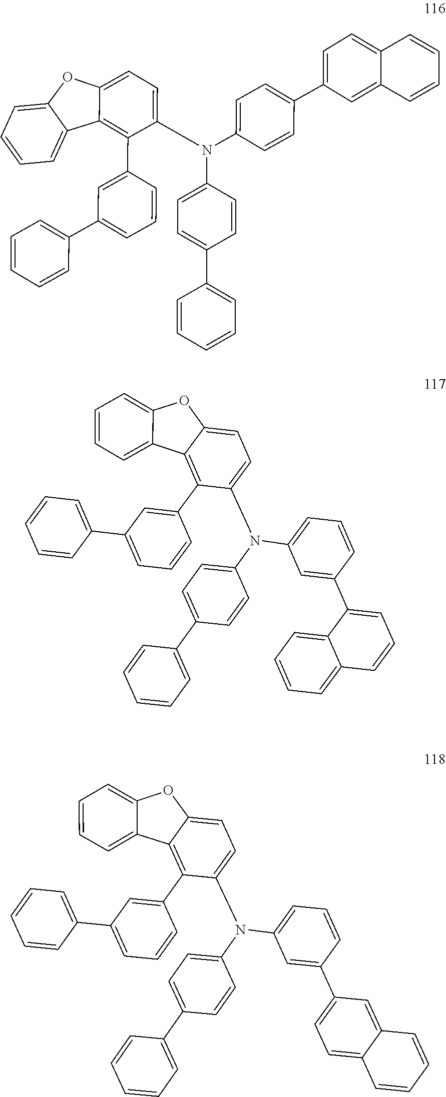 [SMILES: c1ccc(-c2ccc(N(c3ccc(-c4ccc5ccccc5c4)cc3)c3ccc4oc5ccccc5c4c3-c3cccc(-c4ccccc4)c3)cc2)cc1 ; c1ccc(-c2ccc(N(c3cccc(-c4cccc5ccccc45)c3)c3ccc4oc5ccccc5c4c3-c3cccc(-c4ccccc4)c3)cc2)cc1 ; c1ccc(-c2ccc(N(c3cccc(-c4ccc5ccccc5c4)c3)c3ccc4oc5ccccc5c4c3-c3cccc(-c4ccccc4)c3)cc2)cc1]

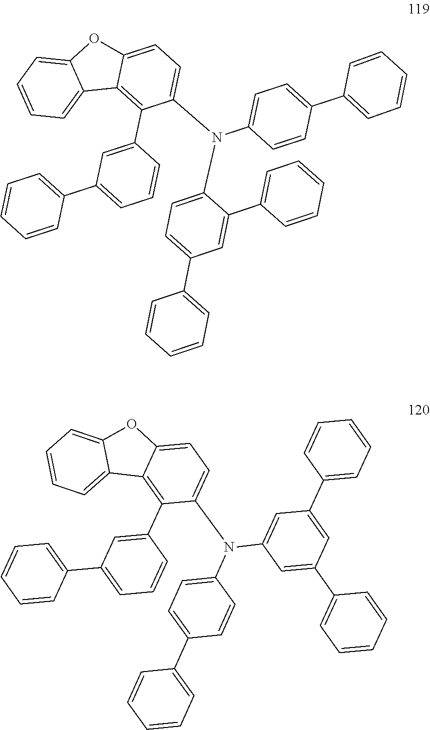 [SMILES: c1ccc(-c2ccc(N(c3ccc(-c4ccccc4)cc3-c3ccccc3)c3ccc4oc5ccccc5c4c3-c3cccc(-c4ccccc4)c3)cc2)cc1 ; c1ccc(-c2ccc(N(c3cc(-c4ccccc4)cc(-c4ccccc4)c3)c3ccc4oc5ccccc5c4c3-c3cccc(-c4ccccc4)c3)cc2)cc1]

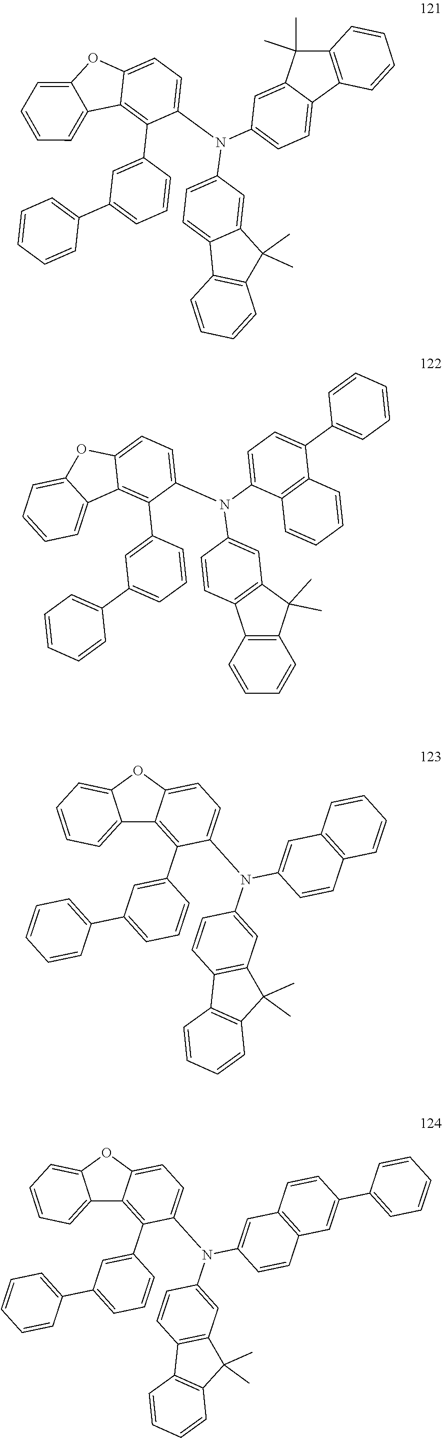 [SMILES: CC1(C)c2ccccc2-c2ccc(N(c3ccc4c(c3)C(C)(C)c3ccccc3-4)c3ccc4oc5ccccc5c4c3-c3cccc(-c4ccccc4)c3)cc21 ; CC1(C)c2ccccc2-c2ccc(N(c3ccc4oc5ccccc5c4c3-c3cccc(-c4ccccc4)c3)c3ccc(-c4ccccc4)c4ccccc34)cc21 ; CC1(C)c2ccccc2-c2ccc(N(c3ccc4ccccc4c3)c3ccc4oc5ccccc5c4c3-c3cccc(-c4ccccc4)c3)cc21 ; CC1(C)c2ccccc2-c2ccc(N(c3ccc4cc(-c5ccccc5)ccc4c3)c3ccc4oc5ccccc5c4c3-c3cccc(-c4ccccc4)c3)cc21]

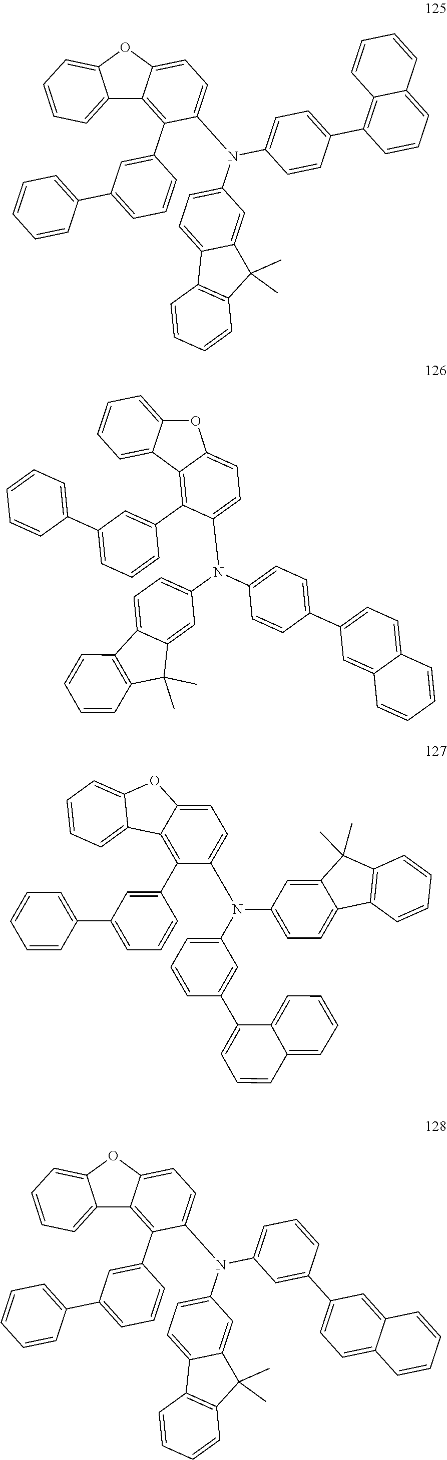 [SMILES: CC1(C)c2ccccc2-c2ccc(N(c3ccc(-c4cccc5ccccc45)cc3)c3ccc4oc5ccccc5c4c3-c3cccc(-c4ccccc4)c3)cc21 ; CC1(C)c2ccccc2-c2ccc(N(c3ccc(-c4ccc5ccccc5c4)cc3)c3ccc4oc5ccccc5c4c3-c3cccc(-c4ccccc4)c3)cc21 ; CC1(C)c2ccccc2-c2ccc(N(c3cccc(-c4cccc5ccccc45)c3)c3ccc4oc5ccccc5c4c3-c3cccc(-c4ccccc4)c3)cc21 ; CC1(C)c2ccccc2-c2ccc(N(c3cccc(-c4ccc5ccccc5c4)c3)c3ccc4oc5ccccc5c4c3-c3cccc(-c4ccccc4)c3)cc21]

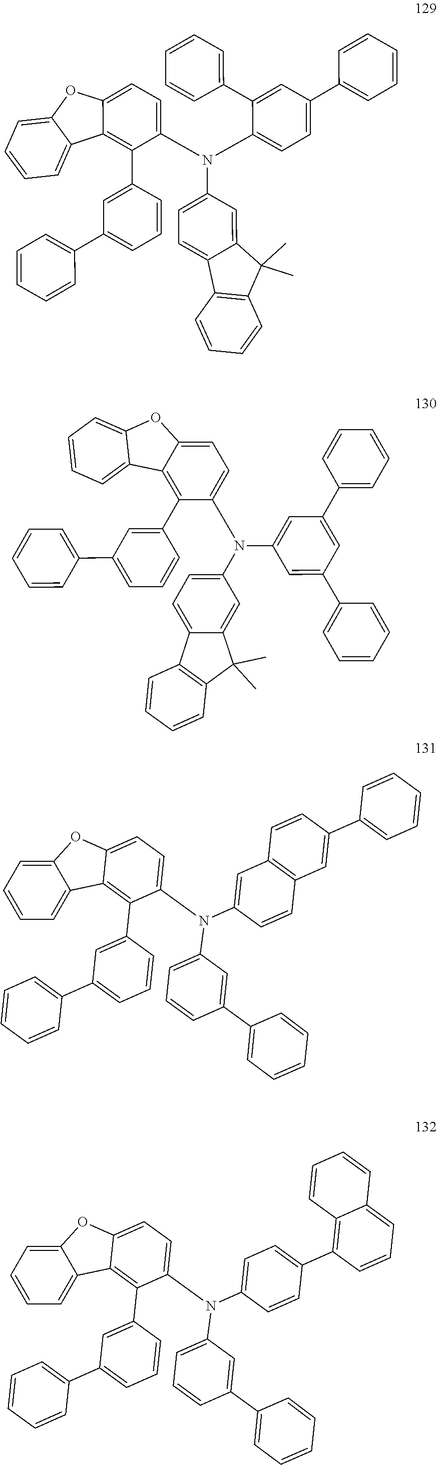 [SMILES: CC1(C)c2ccccc2-c2ccc(N(c3ccc(-c4ccccc4)cc3-c3ccccc3)c3ccc4oc5ccccc5c4c3-c3cccc(-c4ccccc4)c3)cc21 ; CC1(C)c2ccccc2-c2ccc(N(c3cc(-c4ccccc4)cc(-c4ccccc4)c3)c3ccc4oc5ccccc5c4c3-c3cccc(-c4ccccc4)c3)cc21 ; c1ccc(-c2cccc(-c3c(N(c4cccc(-c5ccccc5)c4)c4ccc5cc(-c6ccccc6)ccc5c4)ccc4oc5ccccc5c34)c2)cc1 ; c1ccc(-c2cccc(-c3c(N(c4ccc(-c5cccc6ccccc56)cc4)c4cccc(-c5ccccc5)c4)ccc4oc5ccccc5c34)c2)cc1]

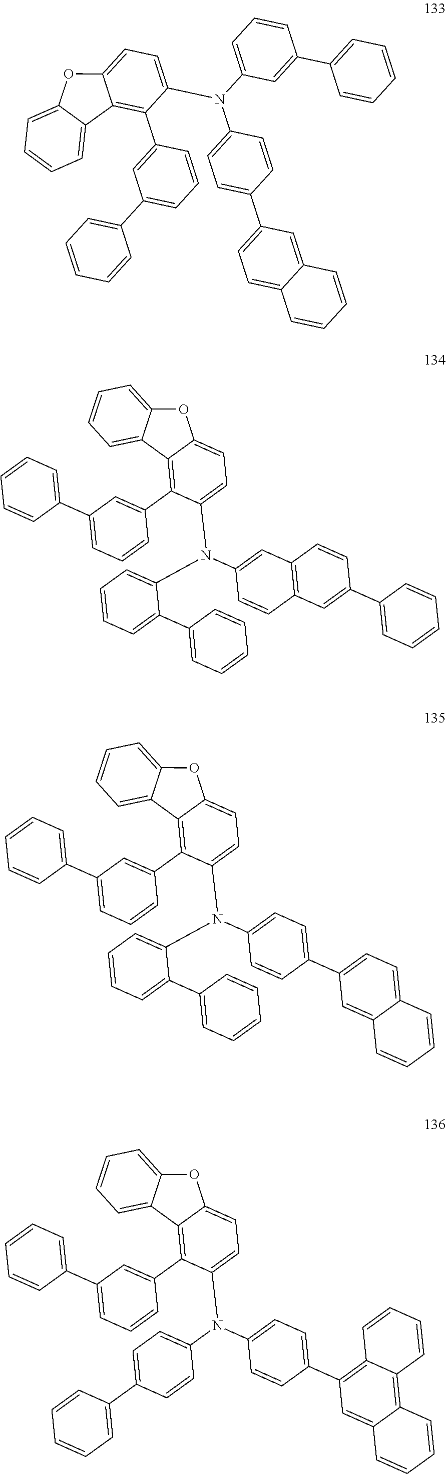 [SMILES: c1ccc(-c2cccc(-c3c(N(c4ccc(-c5ccc6ccccc6c5)cc4)c4cccc(-c5ccccc5)c4)ccc4oc5ccccc5c34)c2)cc1 ; c1ccc(-c2cccc(-c3c(N(c4ccc5cc(-c6ccccc6)ccc5c4)c4ccccc4-c4ccccc4)ccc4oc5ccccc5c34)c2)cc1 ; c1ccc(-c2cccc(-c3c(N(c4ccc(-c5ccc6ccccc6c5)cc4)c4ccccc4-c4ccccc4)ccc4oc5ccccc5c34)c2)cc1 ; c1ccc(-c2ccc(N(c3ccc(-c4cc5ccccc5c5ccccc45)cc3)c3ccc4oc5ccccc5c4c3-c3cccc(-c4ccccc4)c3)cc2)cc1]

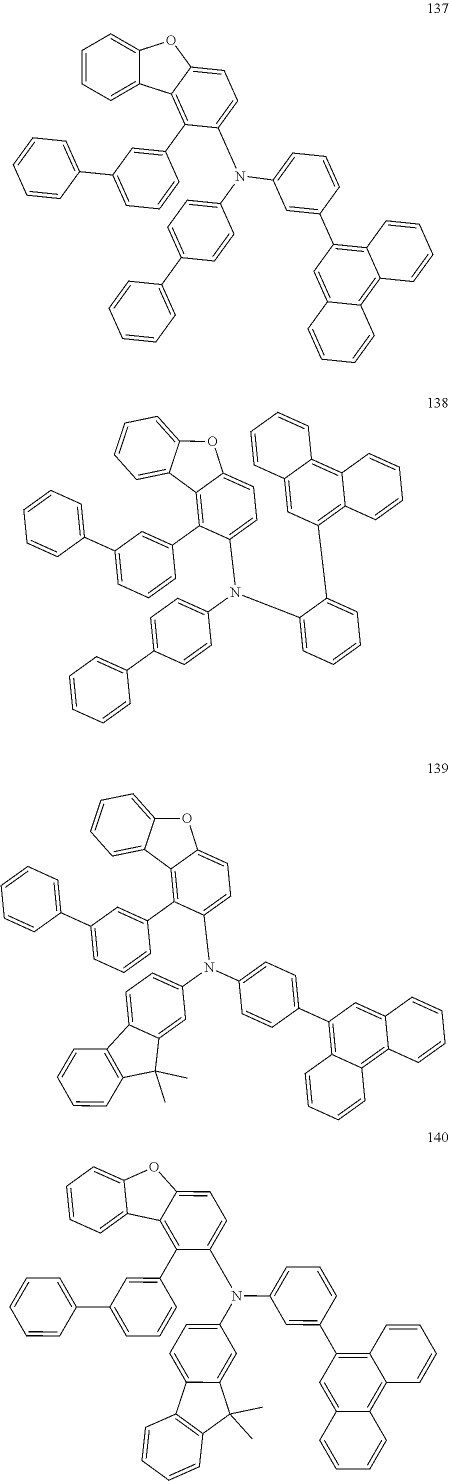 [SMILES: c1ccc(-c2ccc(N(c3cccc(-c4cc5ccccc5c5ccccc45)c3)c3ccc4oc5ccccc5c4c3-c3cccc(-c4ccccc4)c3)cc2)cc1 ; c1ccc(-c2ccc(N(c3ccccc3-c3cc4ccccc4c4ccccc34)c3ccc4oc5ccccc5c4c3-c3cccc(-c4ccccc4)c3)cc2)cc1 ; CC1(C)c2ccccc2-c2ccc(N(c3ccc(-c4cc5ccccc5c5ccccc45)cc3)c3ccc4oc5ccccc5c4c3-c3cccc(-c4ccccc4)c3)cc21 ; CC1(C)c2ccccc2-c2ccc(N(c3cccc(-c4cc5ccccc5c5ccccc45)c3)c3ccc4oc5ccccc5c4c3-c3cccc(-c4ccccc4)c3)cc21]

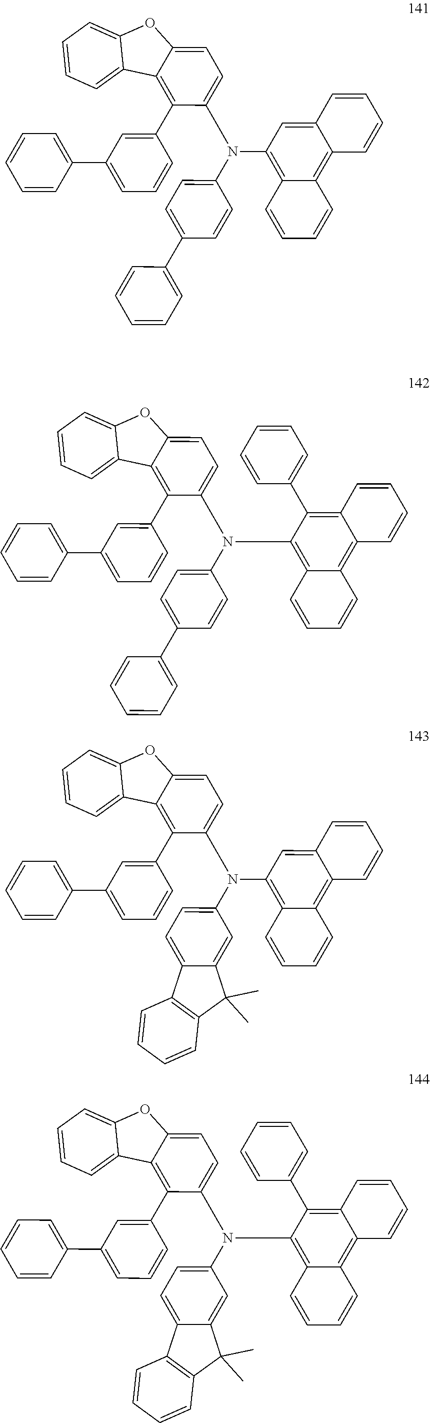

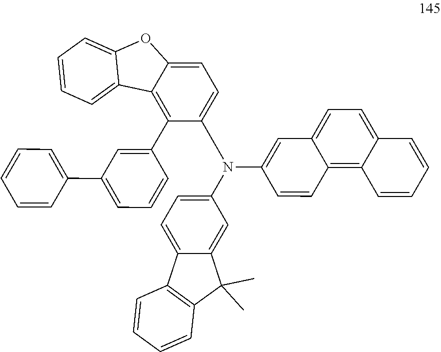

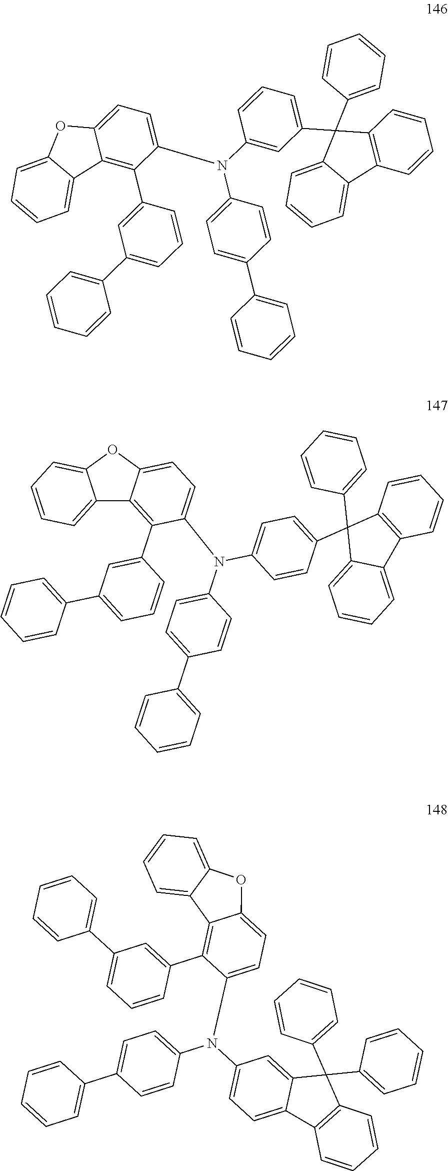 [SMILES: c1ccc(-c2ccc(N(c3cccc(C4(c5ccccc5)c5ccccc5-c5ccccc54)c3)c3ccc4oc5ccccc5c4c3-c3cccc(-c4ccccc4)c3)cc2)cc1 ; c1ccc(-c2ccc(N(c3ccc(C4(c5ccccc5)c5ccccc5-c5ccccc54)cc3)c3ccc4oc5ccccc5c4c3-c3cccc(-c4ccccc4)c3)cc2)cc1 ; c1ccc(-c2ccc(N(c3ccc4c(c3)C(c3ccccc3)(c3ccccc3)c3ccccc3-4)c3ccc4oc5ccccc5c4c3-c3cccc(-c4ccccc4)c3)cc2)cc1]

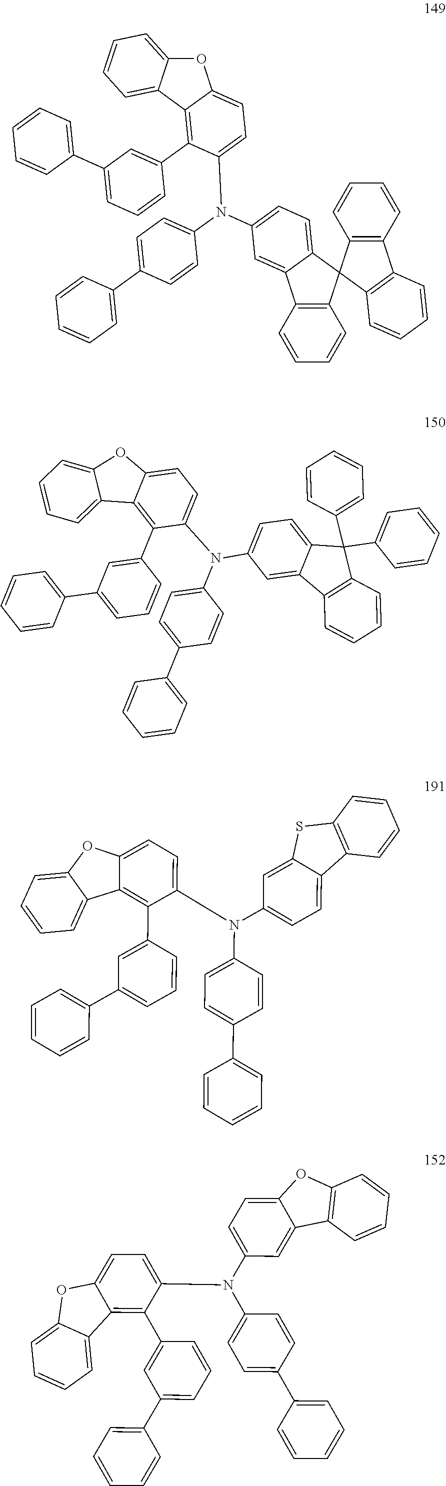 [SMILES: c1ccc(-c2ccc(N(c3ccc4c(c3)-c3ccccc3C43c4ccccc4-c4ccccc43)c3ccc4oc5ccccc5c4c3-c3cccc(-c4ccccc4)c3)cc2)cc1 ; c1ccc(-c2ccc(N(c3ccc4c(c3)-c3ccccc3C4(c3ccccc3)c3ccccc3)c3ccc4oc5ccccc5c4c3-c3cccc(-c4ccccc4)c3)cc2)cc1 ; c1ccc(-c2ccc(N(c3ccc4c(c3)sc3ccccc34)c3ccc4oc5ccccc5c4c3-c3cccc(-c4ccccc4)c3)cc2)cc1 ; c1ccc(-c2ccc(N(c3ccc4oc5ccccc5c4c3)c3ccc4oc5ccccc5c4c3-c3cccc(-c4ccccc4)c3)cc2)cc1]

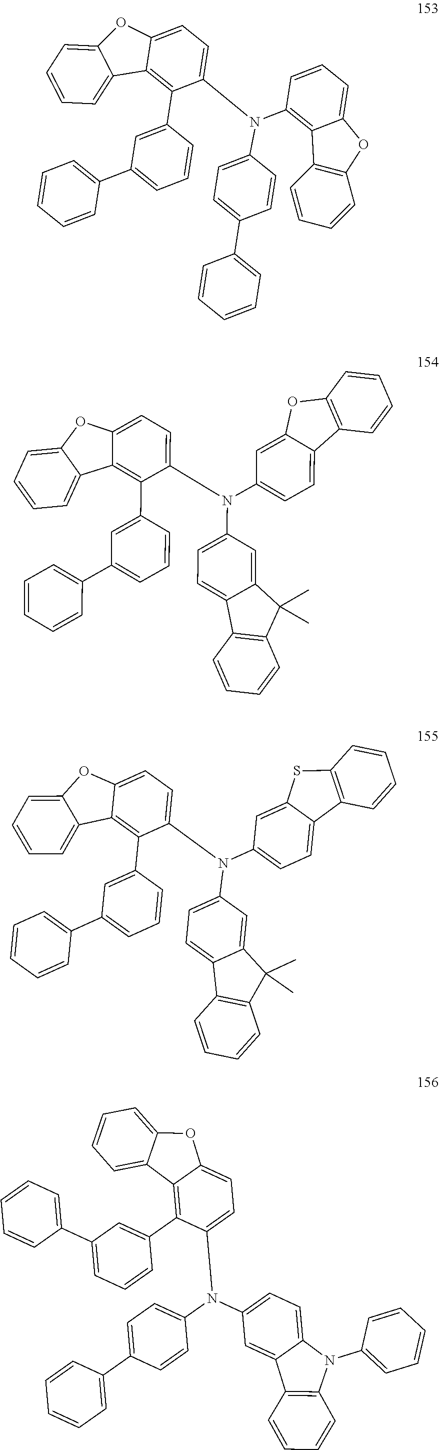 [SMILES: c1ccc(-c2ccc(N(c3ccc4oc5ccccc5c4c3-c3cccc(-c4ccccc4)c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3cccc(-c4ccccc4)c3)cc21 ; CC1(C)c2ccccc2-c2ccc(N(c3ccc4c(c3)sc3ccccc34)c3ccc4oc5ccccc5c4c3-c3cccc(-c4ccccc4)c3)cc21 ; c1ccc(-c2ccc(N(c3ccc4c(c3)c3ccccc3n4-c3ccccc3)c3ccc4oc5ccccc5c4c3-c3cccc(-c4ccccc4)c3)cc2)cc1]

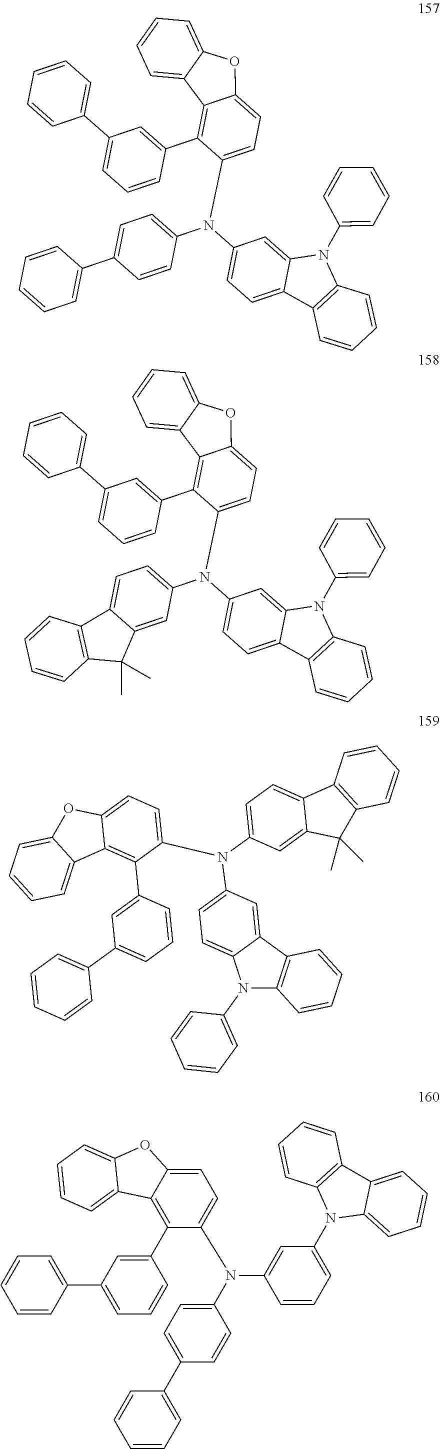 [SMILES: c1ccc(-c2ccc(N(c3ccc4c5ccccc5n(-c5ccccc5)c4c3)c3ccc4oc5ccccc5c4c3-c3cccc(-c4ccccc4)c3)cc2)cc1 ; CC1(C)c2ccccc2-c2ccc(N(c3ccc4c5ccccc5n(-c5ccccc5)c4c3)c3ccc4oc5ccccc5c4c3-c3cccc(-c4ccccc4)c3)cc21 ; CC1(C)c2ccccc2-c2ccc(N(c3ccc4c(c3)c3ccccc3n4-c3ccccc3)c3ccc4oc5ccccc5c4c3-c3cccc(-c4ccccc4)c3)cc21 ; c1ccc(-c2ccc(N(c3cccc(-n4c5ccccc5c5ccccc54)c3)c3ccc4oc5ccccc5c4c3-c3cccc(-c4ccccc4)c3)cc2)cc1]

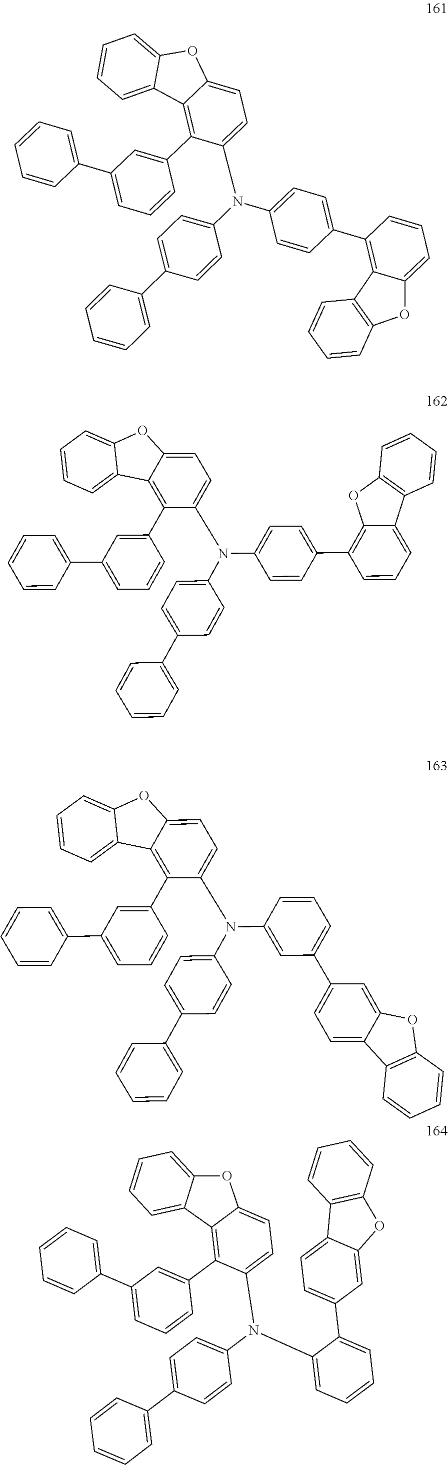 [SMILES: c1ccc(-c2ccc(N(c3ccc(-c4cccc5oc6ccccc6c45)cc3)c3ccc4oc5ccccc5c4c3-c3cccc(-c4ccccc4)c3)cc2)cc1 ; c1ccc(-c2ccc(N(c3ccc(-c4cccc5c4oc4ccccc45)cc3)c3ccc4oc5ccccc5c4c3-c3cccc(-c4ccccc4)c3)cc2)cc1 ; c1ccc(-c2ccc(N(c3cccc(-c4ccc5c(c4)oc4ccccc45)c3)c3ccc4oc5ccccc5c4c3-c3cccc(-c4ccccc4)c3)cc2)cc1 ; c1ccc(-c2ccc(N(c3ccccc3-c3ccc4c(c3)oc3ccccc34)c3ccc4oc5ccccc5c4c3-c3cccc(-c4ccccc4)c3)cc2)cc1]

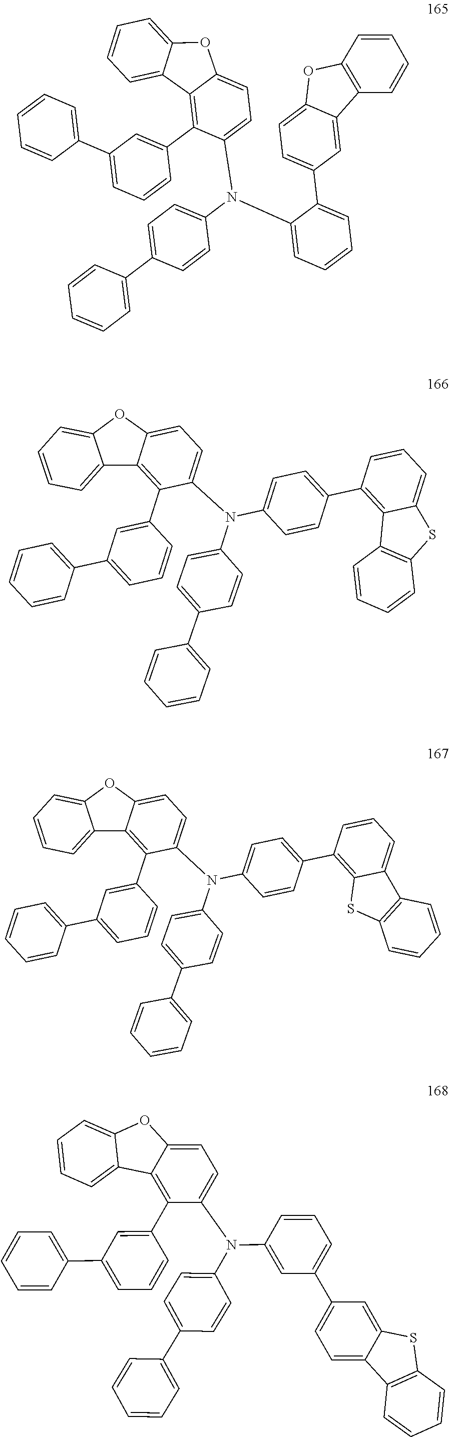 [SMILES: c1ccc(-c2ccc(N(c3ccccc3-c3ccc4oc5ccccc5c4c3)c3ccc4oc5ccccc5c4c3-c3cccc(-c4ccccc4)c3)cc2)cc1 ; c1ccc(-c2ccc(N(c3ccc(-c4cccc5sc6ccccc6c45)cc3)c3ccc4oc5ccccc5c4c3-c3cccc(-c4ccccc4)c3)cc2)cc1 ; c1ccc(-c2ccc(N(c3ccc(-c4cccc5c4sc4ccccc45)cc3)c3ccc4oc5ccccc5c4c3-c3cccc(-c4ccccc4)c3)cc2)cc1 ; c1ccc(-c2ccc(N(c3cccc(-c4ccc5c(c4)sc4ccccc45)c3)c3ccc4oc5ccccc5c4c3-c3cccc(-c4ccccc4)c3)cc2)cc1]

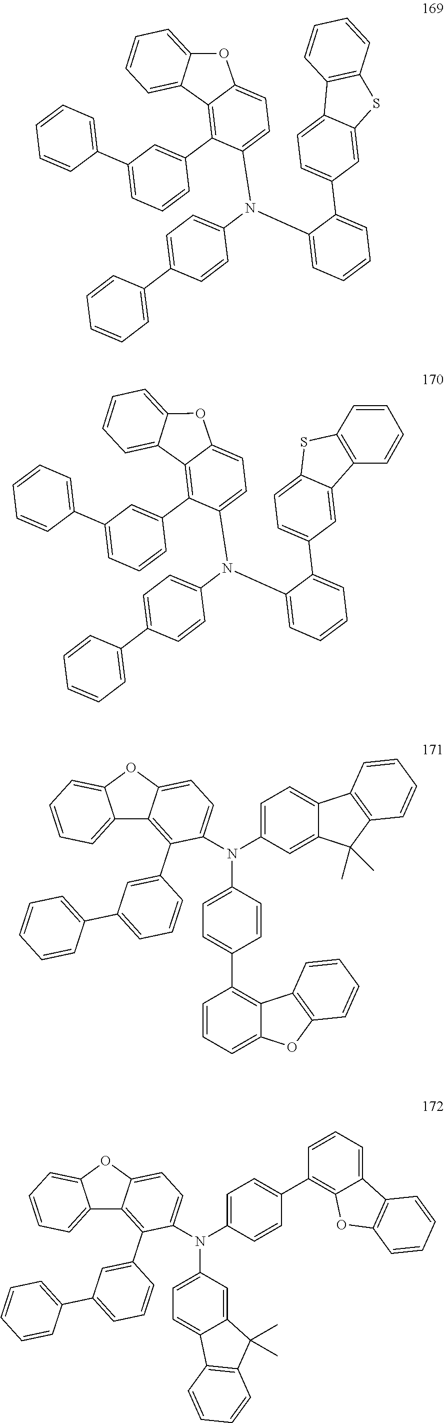 [SMILES: c1ccc(-c2ccc(N(c3ccccc3-c3ccc4c(c3)sc3ccccc34)c3ccc4oc5ccccc5c4c3-c3cccc(-c4ccccc4)c3)cc2)cc1 ; c1ccc(-c2ccc(N(c3ccccc3-c3ccc4sc5ccccc5c4c3)c3ccc4oc5ccccc5c4c3-c3cccc(-c4ccccc4)c3)cc2)cc1 ; CC1(C)c2ccccc2-c2ccc(N(c3ccc(-c4cccc5oc6ccccc6c45)cc3)c3ccc4oc5ccccc5c4c3-c3cccc(-c4ccccc4)c3)cc21 ; CC1(C)c2ccccc2-c2ccc(N(c3ccc(-c4cccc5c4oc4ccccc45)cc3)c3ccc4oc5ccccc5c4c3-c3cccc(-c4ccccc4)c3)cc21]

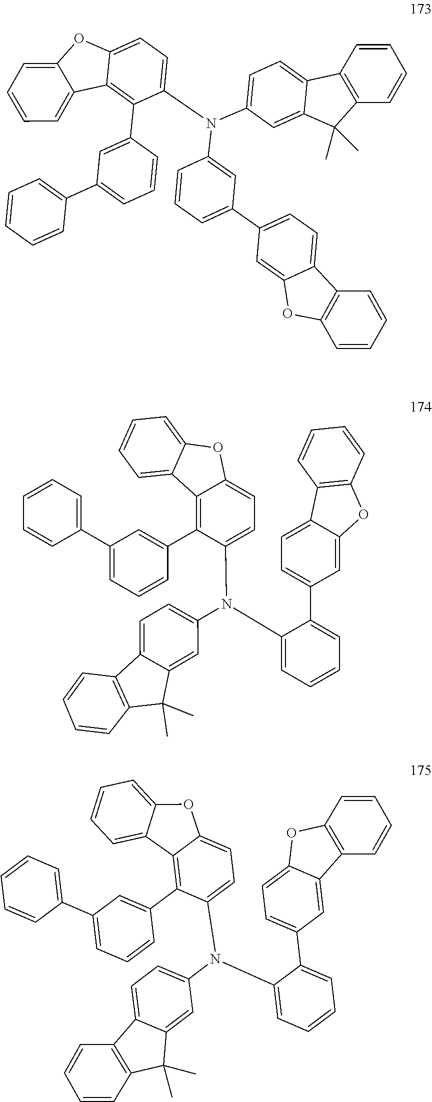 [SMILES: CC1(C)c2ccccc2-c2ccc(N(c3cccc(-c4ccc5c(c4)oc4ccccc45)c3)c3ccc4oc5ccccc5c4c3-c3cccc(-c4ccccc4)c3)cc21 ; CC1(C)c2ccccc2-c2ccc(N(c3ccccc3-c3ccc4c(c3)oc3ccccc34)c3ccc4oc5ccccc5c4c3-c3cccc(-c4ccccc4)c3)cc21 ; CC1(C)c2ccccc2-c2ccc(N(c3ccccc3-c3ccc4oc5ccccc5c4c3)c3ccc4oc5ccccc5c4c3-c3cccc(-c4ccccc4)c3)cc21]

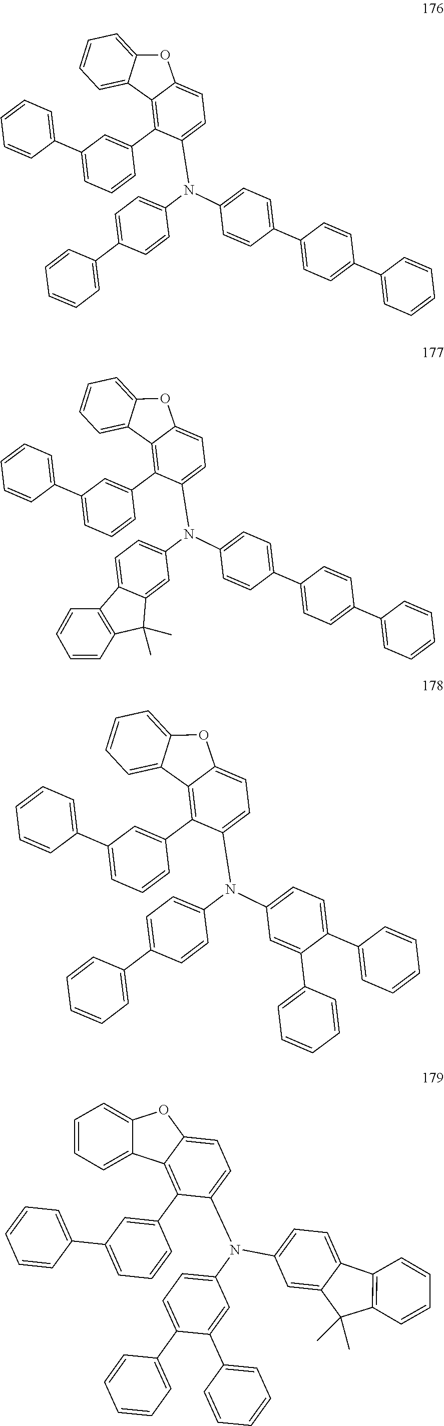 [SMILES: c1ccc(-c2ccc(-c3ccc(N(c4ccc(-c5ccccc5)cc4)c4ccc5oc6ccccc6c5c4-c4cccc(-c5ccccc5)c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc(-c4ccccc4)c3)cc21 ; c1ccc(-c2ccc(N(c3ccc(-c4ccccc4)c(-c4ccccc4)c3)c3ccc4oc5ccccc5c4c3-c3cccc(-c4ccccc4)c3)cc2)cc1 ; CC1(C)c2ccccc2-c2ccc(N(c3ccc(-c4ccccc4)c(-c4ccccc4)c3)c3ccc4oc5ccccc5c4c3-c3cccc(-c4ccccc4)c3)cc21]

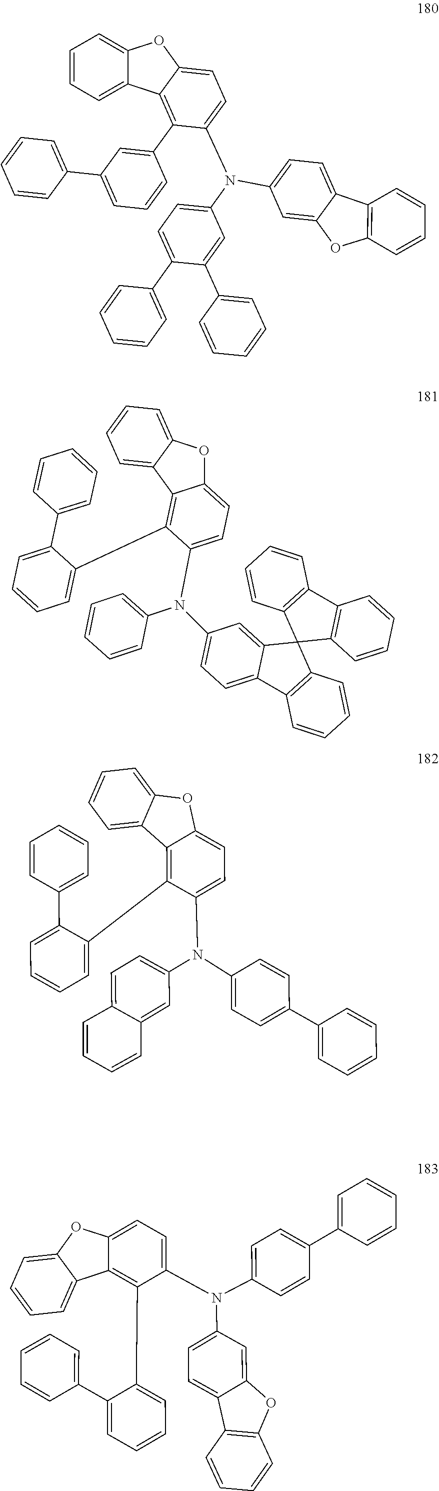 [SMILES: c1ccc(-c2cccc(-c3c(N(c4ccc(-c5ccccc5)c(-c5ccccc5)c4)c4ccc5c(c4)oc4ccccc45)ccc4oc5ccccc5c34)c2)cc1 ; c1ccc(-c2ccccc2-c2c(N(c3ccccc3)c3ccc4c(c3)C3(c5ccccc5-c5ccccc53)c3ccccc3-4)ccc3oc4ccccc4c23)cc1 ; c1ccc(-c2ccc(N(c3ccc4ccccc4c3)c3ccc4oc5ccccc5c4c3-c3ccccc3-c3ccccc3)cc2)cc1 ; c1ccc(-c2ccc(N(c3ccc4c(c3)oc3ccccc34)c3ccc4oc5ccccc5c4c3-c3ccccc3-c3ccccc3)cc2)cc1]

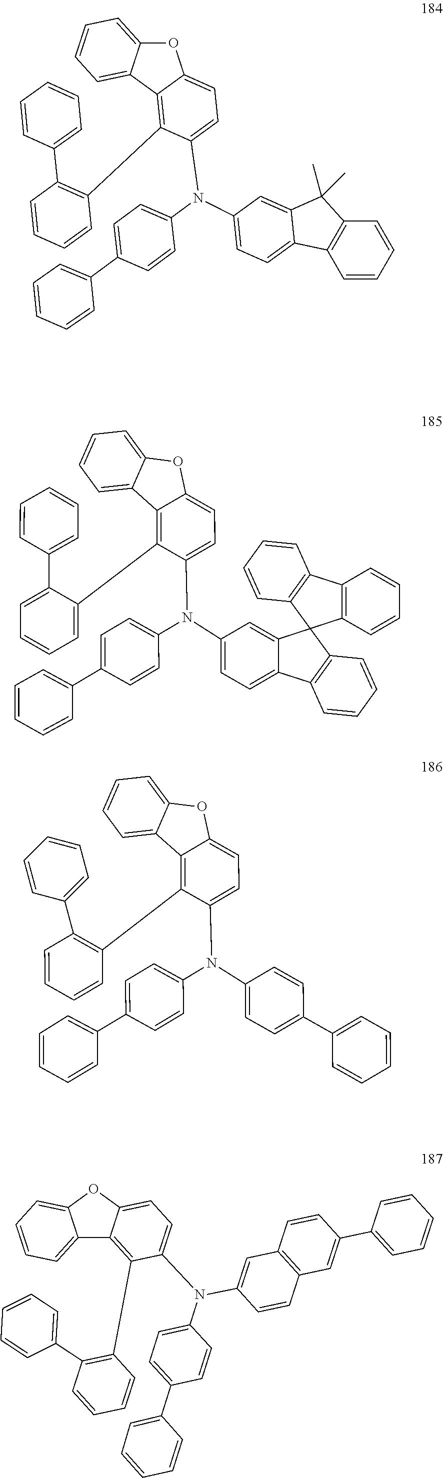 [SMILES: CC1(C)c2ccccc2-c2ccc(N(c3ccc(-c4ccccc4)cc3)c3ccc4oc5ccccc5c4c3-c3ccccc3-c3ccccc3)cc21 ; c1ccc(-c2ccc(N(c3ccc4c(c3)C3(c5ccccc5-c5ccccc53)c3ccccc3-4)c3ccc4oc5ccccc5c4c3-c3ccccc3-c3ccccc3)cc2)cc1 ; c1ccc(-c2ccc(N(c3ccc(-c4ccccc4)cc3)c3ccc4oc5ccccc5c4c3-c3ccccc3-c3ccccc3)cc2)cc1 ; c1ccc(-c2ccc(N(c3ccc4cc(-c5ccccc5)ccc4c3)c3ccc4oc5ccccc5c4c3-c3ccccc3-c3ccccc3)cc2)cc1]

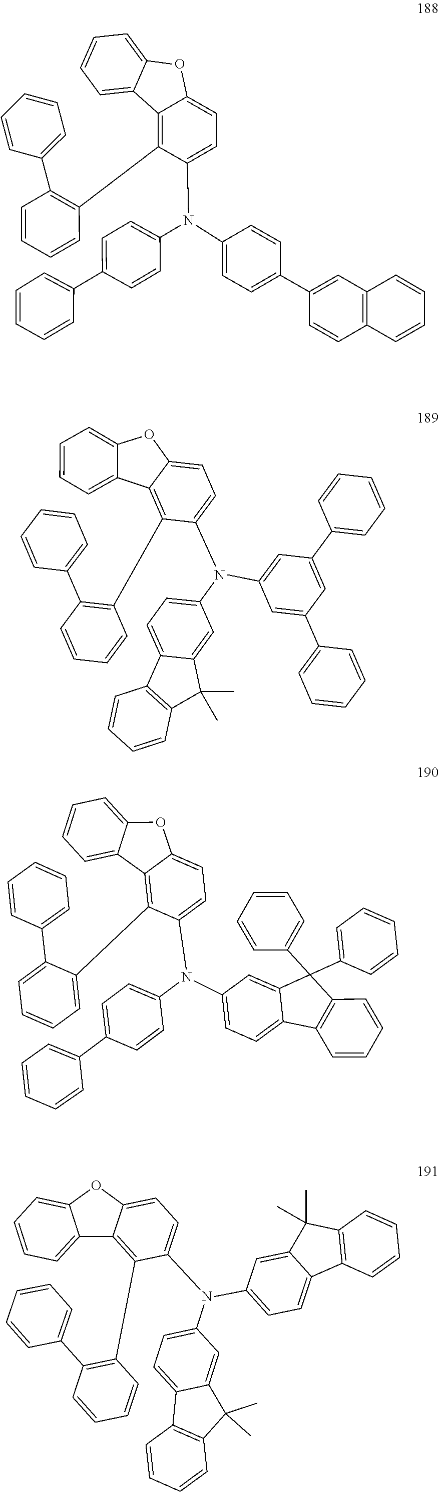 [SMILES: c1ccc(-c2ccc(N(c3ccc(-c4ccc5ccccc5c4)cc3)c3ccc4oc5ccccc5c4c3-c3ccccc3-c3ccccc3)cc2)cc1 ; CC1(C)c2ccccc2-c2ccc(N(c3cc(-c4ccccc4)cc(-c4ccccc4)c3)c3ccc4oc5ccccc5c4c3-c3ccccc3-c3ccccc3)cc21 ; c1ccc(-c2ccc(N(c3ccc4c(c3)C(c3ccccc3)(c3ccccc3)c3ccccc3-4)c3ccc4oc5ccccc5c4c3-c3ccccc3-c3ccccc3)cc2)cc1 ; CC1(C)c2ccccc2-c2ccc(N(c3ccc4c(c3)C(C)(C)c3ccccc3-4)c3ccc4oc5ccccc5c4c3-c3ccccc3-c3ccccc3)cc21]

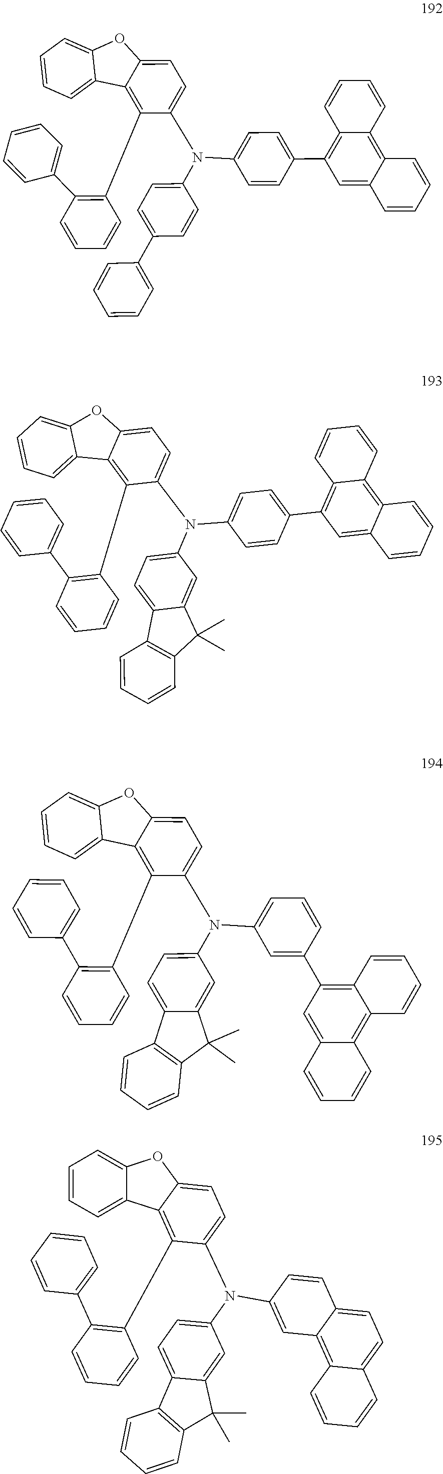 [SMILES: c1ccc(-c2ccc(N(c3ccc(-c4cc5ccccc5c5ccccc45)cc3)c3ccc4oc5ccccc5c4c3-c3ccccc3-c3ccccc3)cc2)cc1 ; CC1(C)c2ccccc2-c2ccc(N(c3ccc(-c4cc5ccccc5c5ccccc45)cc3)c3ccc4oc5ccccc5c4c3-c3ccccc3-c3ccccc3)cc21 ; CC1(C)c2ccccc2-c2ccc(N(c3cccc(-c4cc5ccccc5c5ccccc45)c3)c3ccc4oc5ccccc5c4c3-c3ccccc3-c3ccccc3)cc21 ; CC1(C)c2ccccc2-c2ccc(N(c3ccc4ccc5ccccc5c4c3)c3ccc4oc5ccccc5c4c3-c3ccccc3-c3ccccc3)cc21]

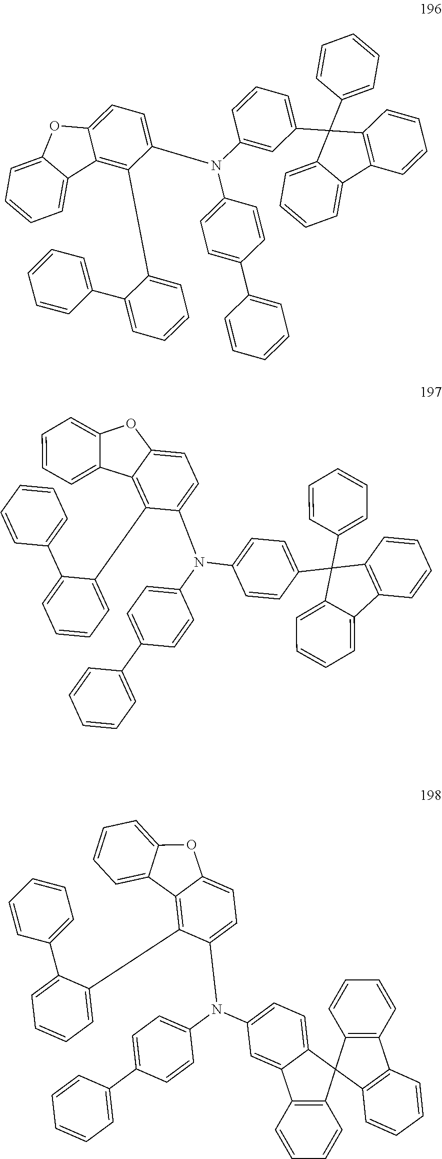 [SMILES: c1ccc(-c2ccc(N(c3cccc(C4(c5ccccc5)c5ccccc5-c5ccccc54)c3)c3ccc4oc5ccccc5c4c3-c3ccccc3-c3ccccc3)cc2)cc1 ; c1ccc(-c2ccc(N(c3ccc(C4(c5ccccc5)c5ccccc5-c5ccccc54)cc3)c3ccc4oc5ccccc5c4c3-c3ccccc3-c3ccccc3)cc2)cc1 ; c1ccc(-c2ccc(N(c3ccc4c(c3)-c3ccccc3C43c4ccccc4-c4ccccc43)c3ccc4oc5ccccc5c4c3-c3ccccc3-c3ccccc3)cc2)cc1]

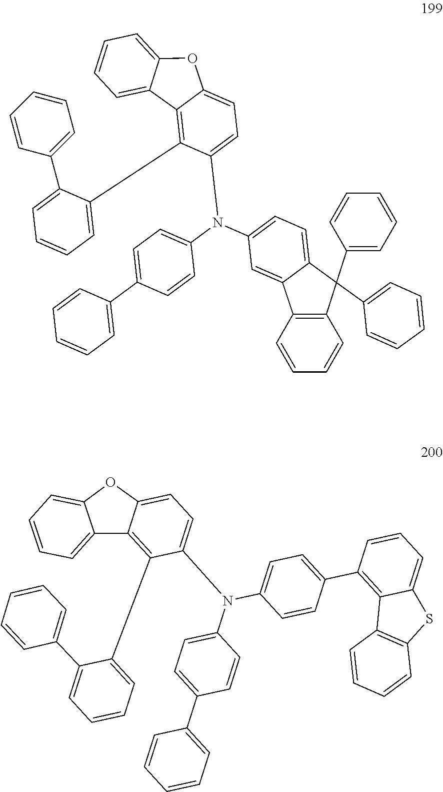 [SMILES: c1ccc(-c2ccc(N(c3ccc4c(c3)-c3ccccc3C4(c3ccccc3)c3ccccc3)c3ccc4oc5ccccc5c4c3-c3ccccc3-c3ccccc3)cc2)cc1 ; c1ccc(-c2ccc(N(c3ccc(-c4cccc5sc6ccccc6c45)cc3)c3ccc4oc5ccccc5c4c3-c3ccccc3-c3ccccc3)cc2)cc1]

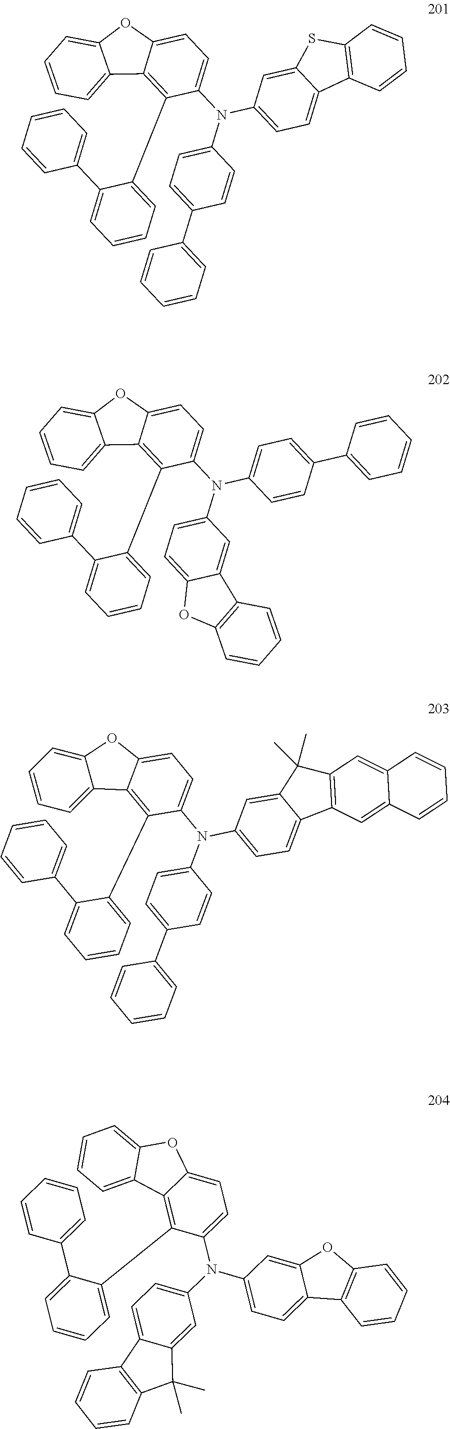 [SMILES: c1ccc(-c2ccc(N(c3ccc4c(c3)sc3ccccc34)c3ccc4oc5ccccc5c4c3-c3ccccc3-c3ccccc3)cc2)cc1 ; c1ccc(-c2ccc(N(c3ccc4oc5ccccc5c4c3)c3ccc4oc5ccccc5c4c3-c3ccccc3-c3ccccc3)cc2)cc1 ; CC1(C)c2cc(N(c3ccc(-c4ccccc4)cc3)c3ccc4oc5ccccc5c4c3-c3ccccc3-c3ccccc3)ccc2-c2cc3ccccc3cc21 ; CC1(C)c2ccccc2-c2ccc(N(c3ccc4c(c3)oc3ccccc34)c3ccc4oc5ccccc5c4c3-c3ccccc3-c3ccccc3)cc21]

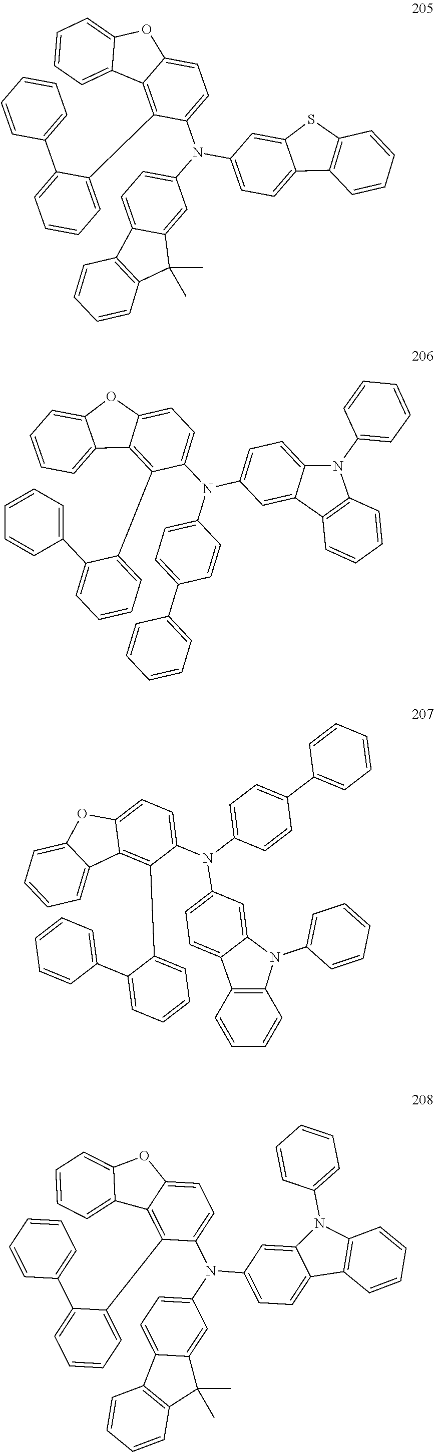 [SMILES: CC1(C)c2ccccc2-c2ccc(N(c3ccc4c(c3)sc3ccccc34)c3ccc4oc5ccccc5c4c3-c3ccccc3-c3ccccc3)cc21 ; c1ccc(-c2ccc(N(c3ccc4c(c3)c3ccccc3n4-c3ccccc3)c3ccc4oc5ccccc5c4c3-c3ccccc3-c3ccccc3)cc2)cc1 ; c1ccc(-c2ccc(N(c3ccc4c5ccccc5n(-c5ccccc5)c4c3)c3ccc4oc5ccccc5c4c3-c3ccccc3-c3ccccc3)cc2)cc1 ; CC1(C)c2ccccc2-c2ccc(N(c3ccc4c5ccccc5n(-c5ccccc5)c4c3)c3ccc4oc5ccccc5c4c3-c3ccccc3-c3ccccc3)cc21]

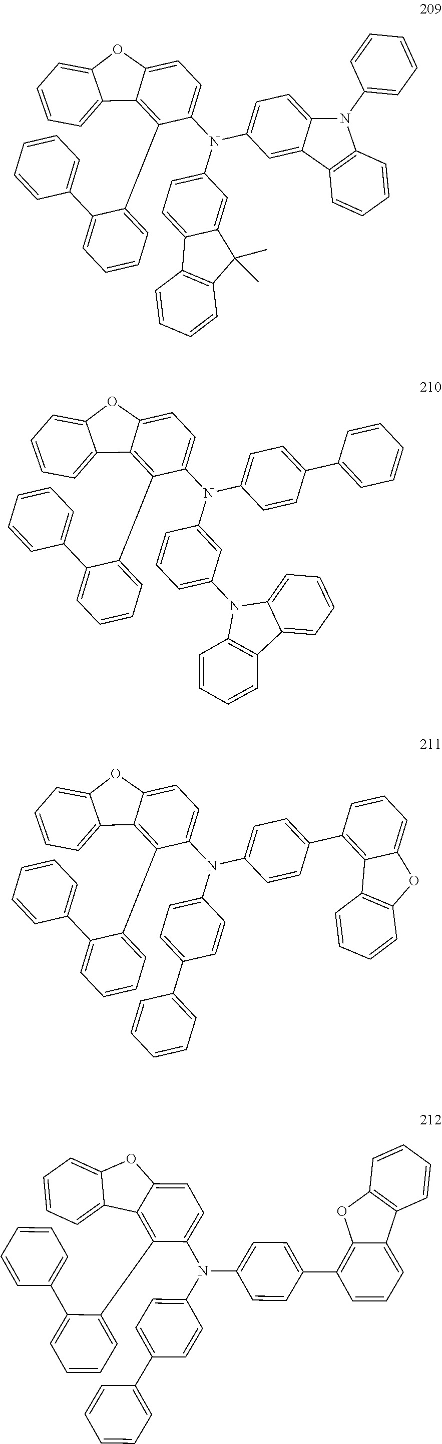 [SMILES: CC1(C)c2ccccc2-c2ccc(N(c3ccc4c(c3)c3ccccc3n4-c3ccccc3)c3ccc4oc5ccccc5c4c3-c3ccccc3-c3ccccc3)cc21 ; c1ccc(-c2ccc(N(c3cccc(-n4c5ccccc5c5ccccc54)c3)c3ccc4oc5ccccc5c4c3-c3ccccc3-c3ccccc3)cc2)cc1 ; c1ccc(-c2ccc(N(c3ccc(-c4cccc5oc6ccccc6c45)cc3)c3ccc4oc5ccccc5c4c3-c3ccccc3-c3ccccc3)cc2)cc1 ; c1ccc(-c2ccc(N(c3ccc(-c4cccc5c4oc4ccccc45)cc3)c3ccc4oc5ccccc5c4c3-c3ccccc3-c3ccccc3)cc2)cc1]

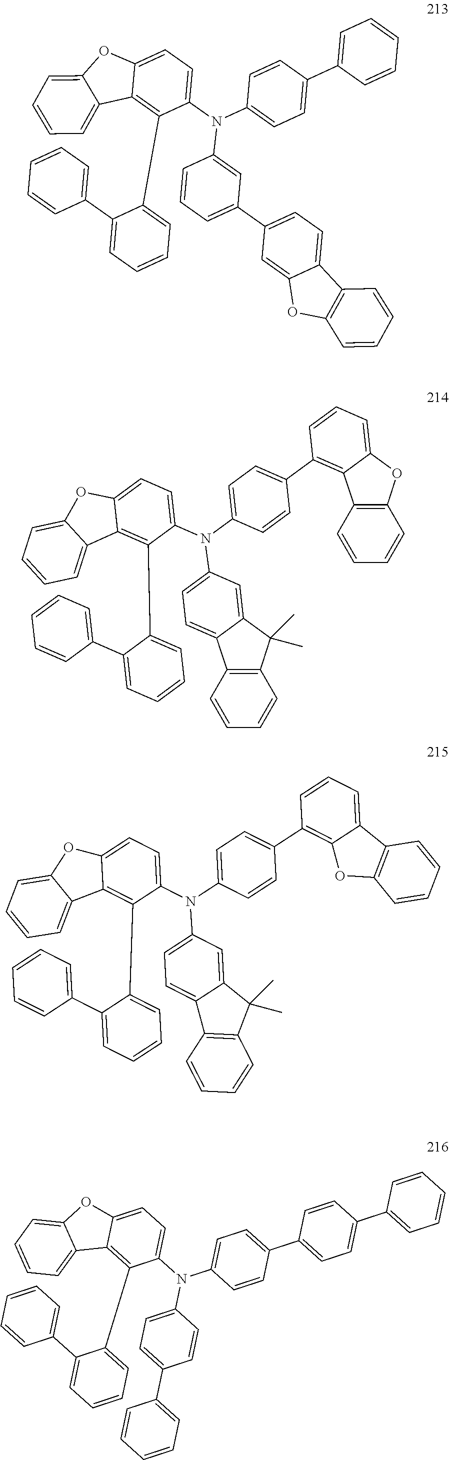 [SMILES: c1ccc(-c2ccc(N(c3cccc(-c4ccc5c(c4)oc4ccccc45)c3)c3ccc4oc5ccccc5c4c3-c3ccccc3-c3ccccc3)cc2)cc1 ; CC1(C)c2ccccc2-c2ccc(N(c3ccc(-c4cccc5oc6ccccc6c45)cc3)c3ccc4oc5ccccc5c4c3-c3ccccc3-c3ccccc3)cc21 ; CC1(C)c2ccccc2-c2ccc(N(c3ccc(-c4cccc5c4oc4ccccc45)cc3)c3ccc4oc5ccccc5c4c3-c3ccccc3-c3ccccc3)cc21 ; c1ccc(-c2ccc(-c3ccc(N(c4ccc(-c5ccccc5)cc4)c4ccc5oc6ccccc6c5c4-c4ccccc4-c4ccccc4)cc3)cc2)cc1]

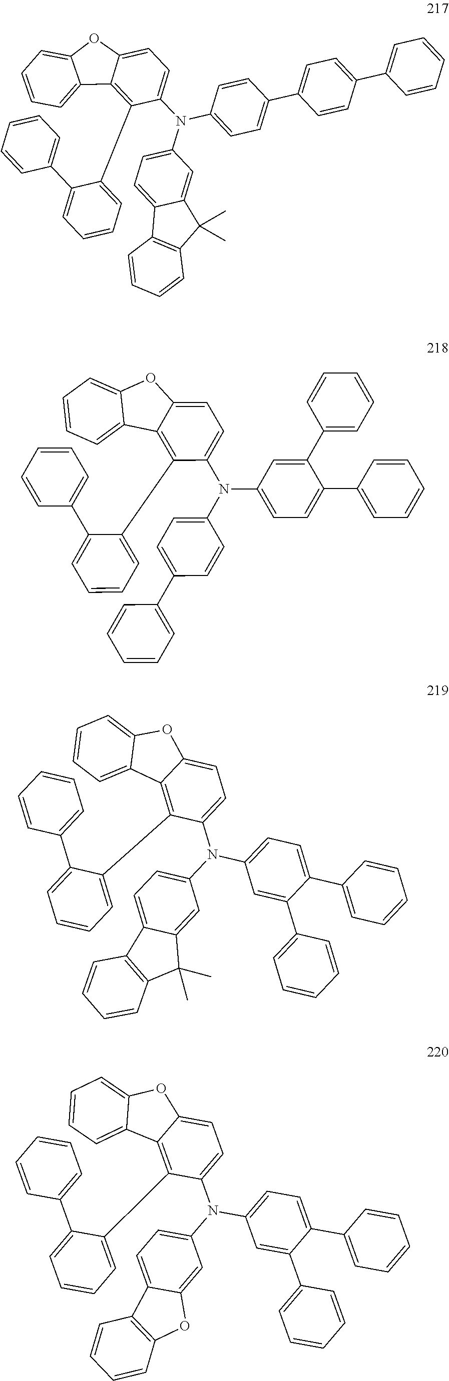 [SMILES: CC1(C)c2ccccc2-c2ccc(N(c3ccc(-c4ccc(-c5ccccc5)cc4)cc3)c3ccc4oc5ccccc5c4c3-c3ccccc3-c3ccccc3)cc21 ; c1ccc(-c2ccc(N(c3ccc(-c4ccccc4)c(-c4ccccc4)c3)c3ccc4oc5ccccc5c4c3-c3ccccc3-c3ccccc3)cc2)cc1 ; CC1(C)c2ccccc2-c2ccc(N(c3ccc(-c4ccccc4)c(-c4ccccc4)c3)c3ccc4oc5ccccc5c4c3-c3ccccc3-c3ccccc3)cc21 ; c1ccc(-c2ccc(N(c3ccc4c(c3)oc3ccccc34)c3ccc4oc5ccccc5c4c3-c3ccccc3-c3ccccc3)cc2-c2ccccc2)cc1]

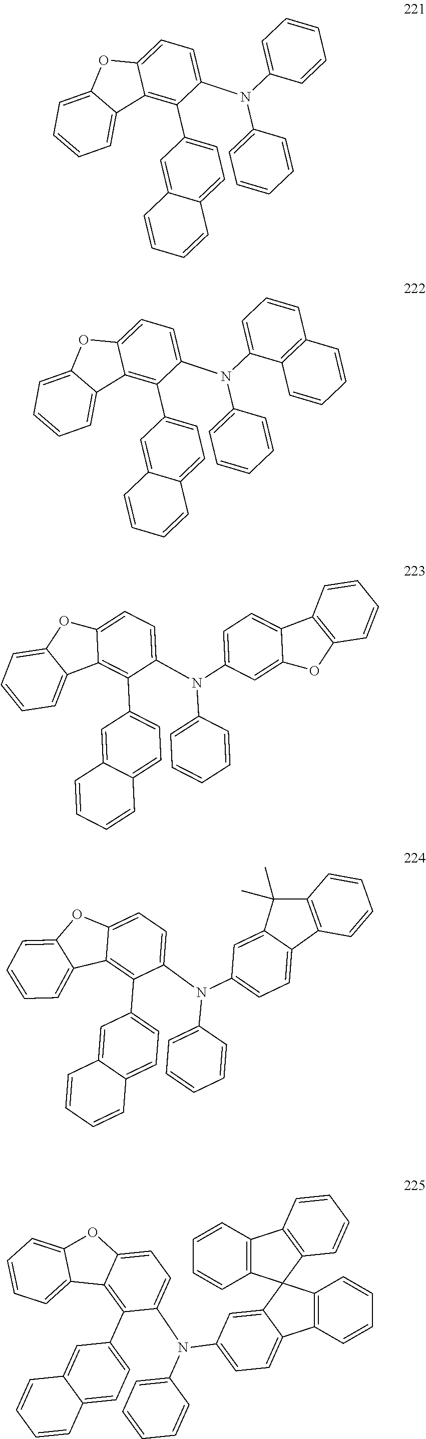 [SMILES: c1ccc(N(c2ccccc2)c2ccc3oc4ccccc4c3c2-c2ccc3ccccc3c2)cc1 ; c1ccc(N(c2ccc3oc4ccccc4c3c2-c2ccc3ccccc3c2)c2cccc3ccccc23)cc1 ; c1ccc(N(c2ccc3c(c2)oc2ccccc23)c2ccc3oc4ccccc4c3c2-c2ccc3ccccc3c2)cc1 ; CC1(C)c2ccccc2-c2ccc(N(c3ccccc3)c3ccc4oc5ccccc5c4c3-c3ccc4ccccc4c3)cc21 ; c1ccc(N(c2ccc3c(c2)C2(c4ccccc4-c4ccccc42)c2ccccc2-3)c2ccc3oc4ccccc4c3c2-c2ccc3ccccc3c2)cc1]

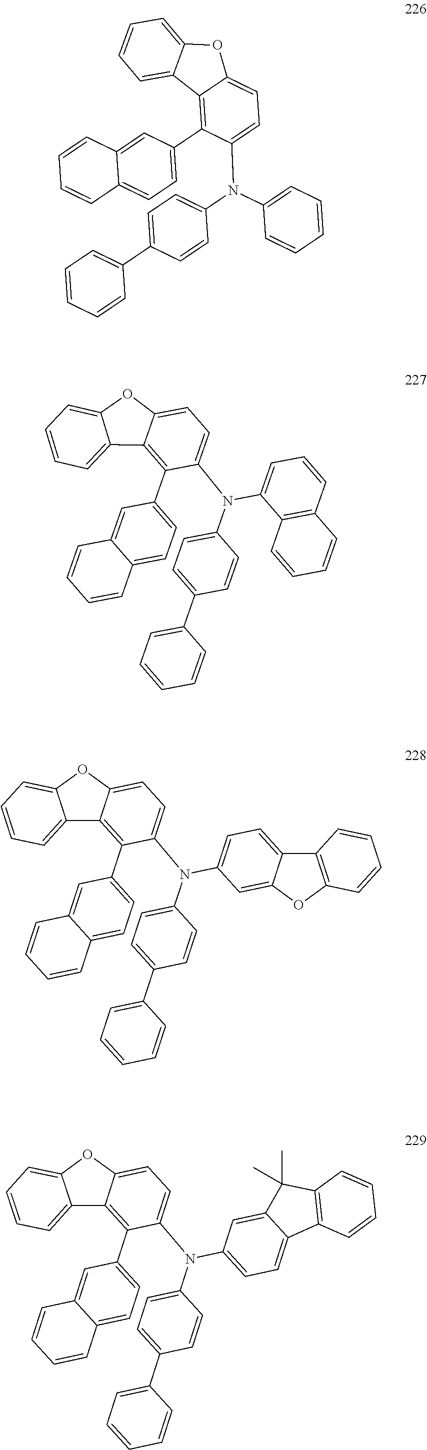 [SMILES: c1ccc(-c2ccc(N(c3ccccc3)c3ccc4oc5ccccc5c4c3-c3ccc4ccccc4c3)cc2)cc1 ; c1ccc(-c2ccc(N(c3ccc4oc5ccccc5c4c3-c3ccc4ccccc4c3)c3cccc4ccccc34)cc2)cc1 ; c1ccc(-c2ccc(N(c3ccc4c(c3)oc3ccccc34)c3ccc4oc5ccccc5c4c3-c3ccc4ccccc4c3)cc2)cc1 ; CC1(C)c2ccccc2-c2ccc(N(c3ccc(-c4ccccc4)cc3)c3ccc4oc5ccccc5c4c3-c3ccc4ccccc4c3)cc21]

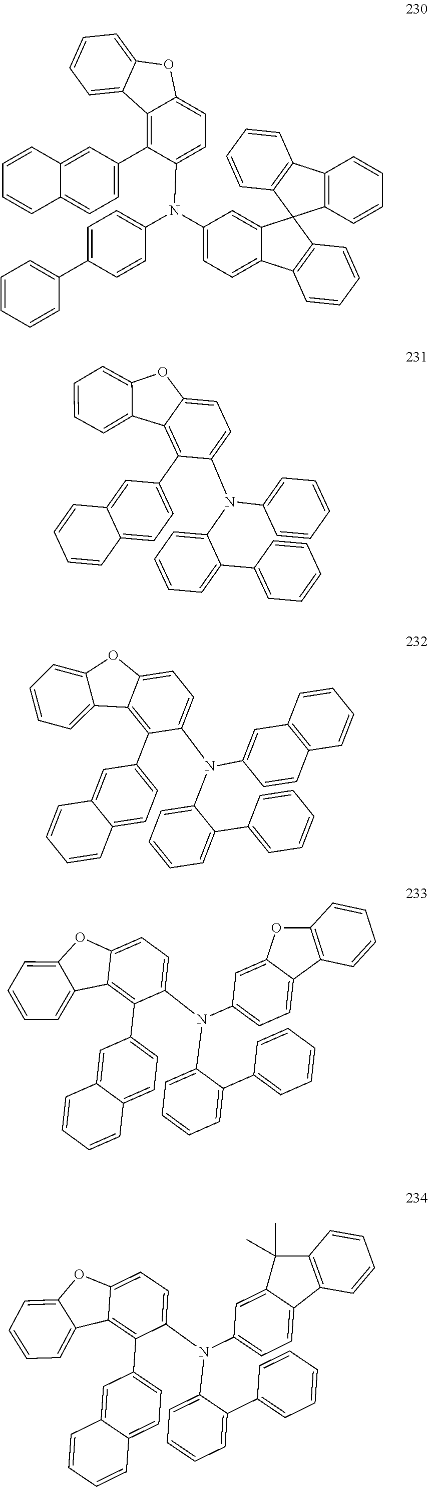 [SMILES: c1ccc(-c2ccc(N(c3ccc4c(c3)C3(c5ccccc5-c5ccccc53)c3ccccc3-4)c3ccc4oc5ccccc5c4c3-c3ccc4ccccc4c3)cc2)cc1 ; c1ccc(-c2ccccc2N(c2ccccc2)c2ccc3oc4ccccc4c3c2-c2ccc3ccccc3c2)cc1 ; c1ccc(-c2ccccc2N(c2ccc3ccccc3c2)c2ccc3oc4ccccc4c3c2-c2ccc3ccccc3c2)cc1 ; c1ccc(-c2ccccc2N(c2ccc3c(c2)oc2ccccc23)c2ccc3oc4ccccc4c3c2-c2ccc3ccccc3c2)cc1 ; CC1(C)c2ccccc2-c2ccc(N(c3ccccc3-c3ccccc3)c3ccc4oc5ccccc5c4c3-c3ccc4ccccc4c3)cc21]

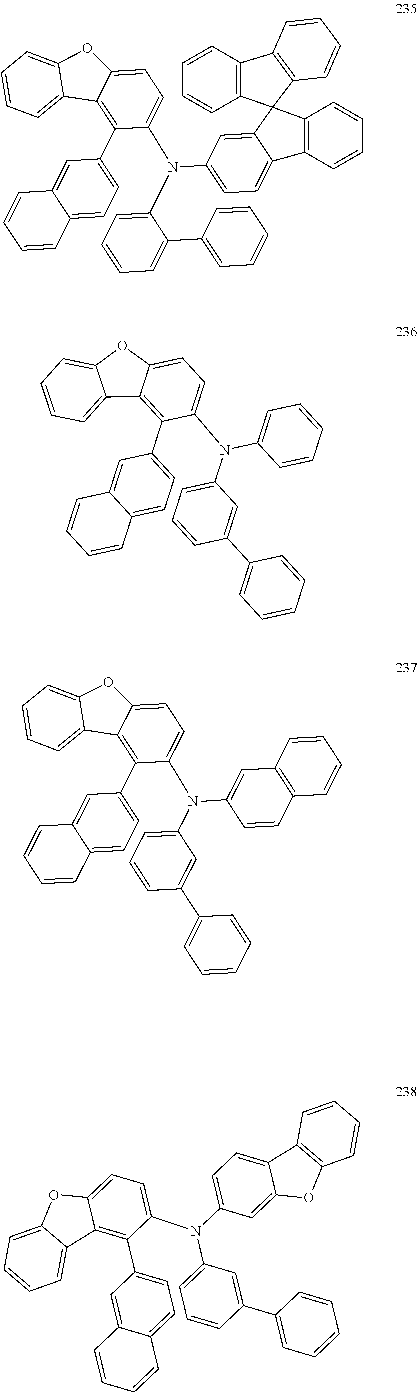 [SMILES: c1ccc(-c2ccccc2N(c2ccc3c(c2)C2(c4ccccc4-c4ccccc42)c2ccccc2-3)c2ccc3oc4ccccc4c3c2-c2ccc3ccccc3c2)cc1 ; c1ccc(-c2cccc(N(c3ccccc3)c3ccc4oc5ccccc5c4c3-c3ccc4ccccc4c3)c2)cc1 ; c1ccc(-c2cccc(N(c3ccc4ccccc4c3)c3ccc4oc5ccccc5c4c3-c3ccc4ccccc4c3)c2)cc1 ; c1ccc(-c2cccc(N(c3ccc4c(c3)oc3ccccc34)c3ccc4oc5ccccc5c4c3-c3ccc4ccccc4c3)c2)cc1]

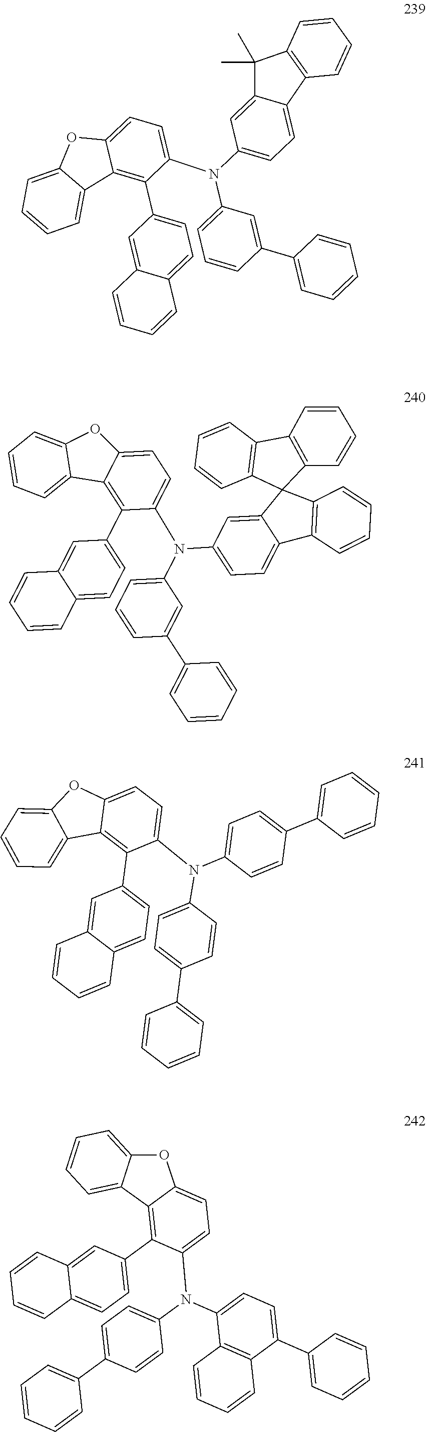 [SMILES: CC1(C)c2ccccc2-c2ccc(N(c3cccc(-c4ccccc4)c3)c3ccc4oc5ccccc5c4c3-c3ccc4ccccc4c3)cc21 ; c1ccc(-c2cccc(N(c3ccc4c(c3)C3(c5ccccc5-c5ccccc53)c3ccccc3-4)c3ccc4oc5ccccc5c4c3-c3ccc4ccccc4c3)c2)cc1 ; c1ccc(-c2ccc(N(c3ccc(-c4ccccc4)cc3)c3ccc4oc5ccccc5c4c3-c3ccc4ccccc4c3)cc2)cc1 ; c1ccc(-c2ccc(N(c3ccc4oc5ccccc5c4c3-c3ccc4ccccc4c3)c3ccc(-c4ccccc4)c4ccccc34)cc2)cc1]

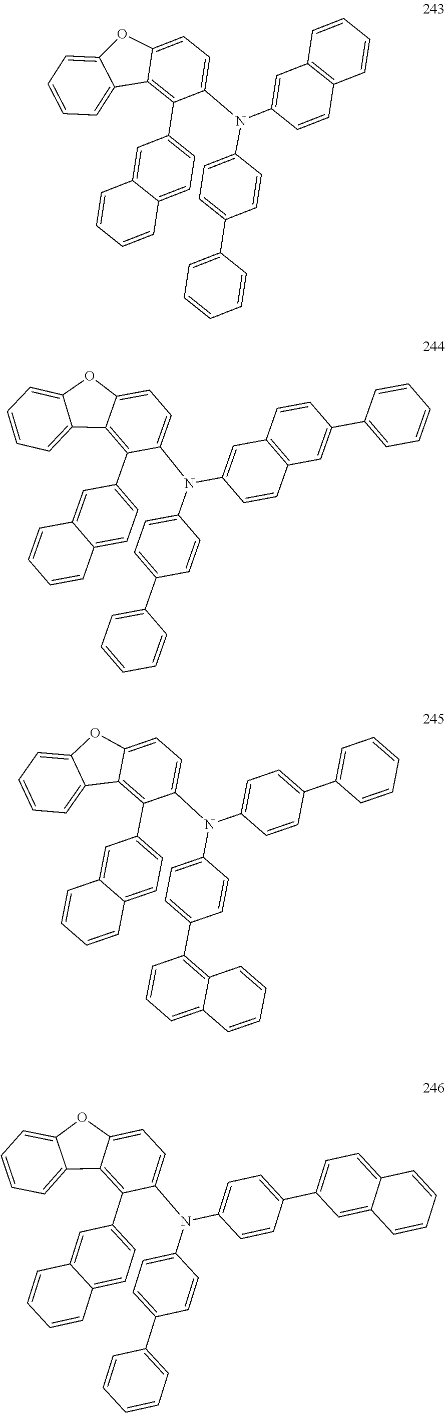 [SMILES: c1ccc(-c2ccc(N(c3ccc4ccccc4c3)c3ccc4oc5ccccc5c4c3-c3ccc4ccccc4c3)cc2)cc1 ; c1ccc(-c2ccc(N(c3ccc4cc(-c5ccccc5)ccc4c3)c3ccc4oc5ccccc5c4c3-c3ccc4ccccc4c3)cc2)cc1 ; c1ccc(-c2ccc(N(c3ccc(-c4cccc5ccccc45)cc3)c3ccc4oc5ccccc5c4c3-c3ccc4ccccc4c3)cc2)cc1 ; c1ccc(-c2ccc(N(c3ccc(-c4ccc5ccccc5c4)cc3)c3ccc4oc5ccccc5c4c3-c3ccc4ccccc4c3)cc2)cc1]

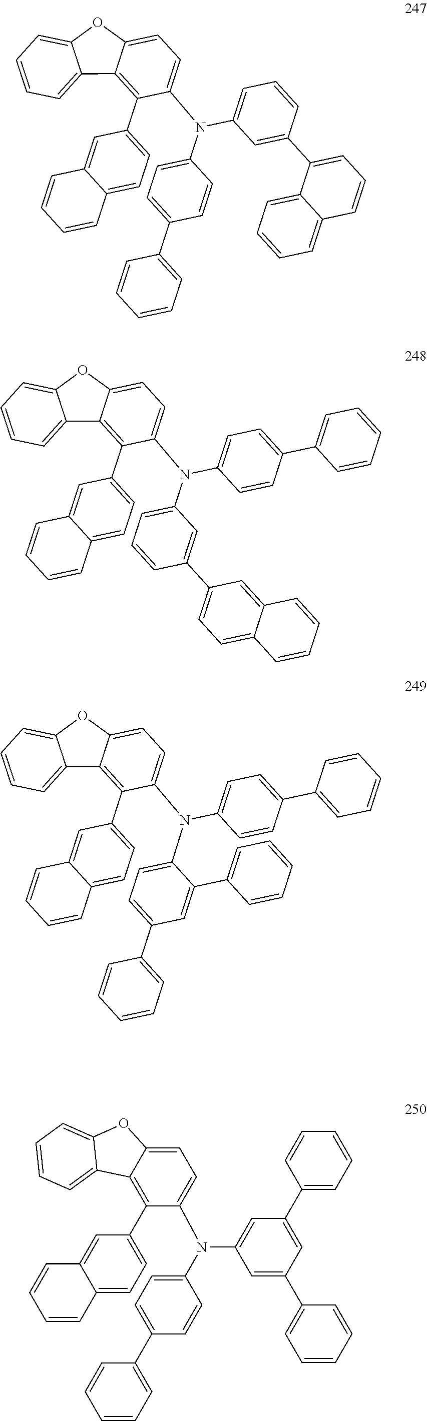 [SMILES: c1ccc(-c2ccc(N(c3cccc(-c4cccc5ccccc45)c3)c3ccc4oc5ccccc5c4c3-c3ccc4ccccc4c3)cc2)cc1 ; c1ccc(-c2ccc(N(c3cccc(-c4ccc5ccccc5c4)c3)c3ccc4oc5ccccc5c4c3-c3ccc4ccccc4c3)cc2)cc1 ; c1ccc(-c2ccc(N(c3ccc(-c4ccccc4)cc3-c3ccccc3)c3ccc4oc5ccccc5c4c3-c3ccc4ccccc4c3)cc2)cc1 ; c1ccc(-c2ccc(N(c3cc(-c4ccccc4)cc(-c4ccccc4)c3)c3ccc4oc5ccccc5c4c3-c3ccc4ccccc4c3)cc2)cc1]

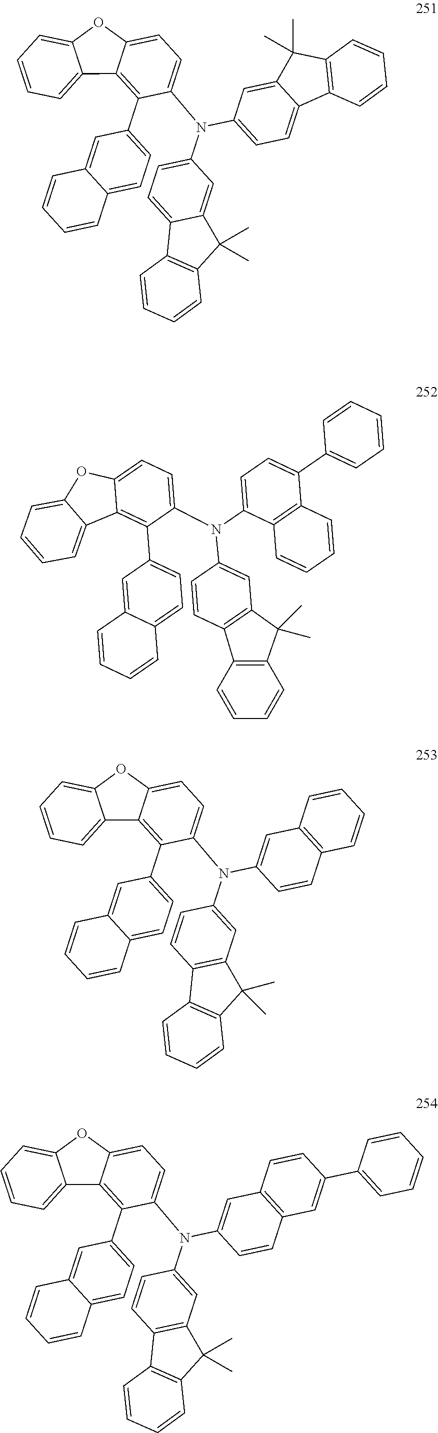 [SMILES: CC1(C)c2ccccc2-c2ccc(N(c3ccc4c(c3)C(C)(C)c3ccccc3-4)c3ccc4oc5ccccc5c4c3-c3ccc4ccccc4c3)cc21 ; CC1(C)c2ccccc2-c2ccc(N(c3ccc4oc5ccccc5c4c3-c3ccc4ccccc4c3)c3ccc(-c4ccccc4)c4ccccc34)cc21 ; CC1(C)c2ccccc2-c2ccc(N(c3ccc4ccccc4c3)c3ccc4oc5ccccc5c4c3-c3ccc4ccccc4c3)cc21 ; CC1(C)c2ccccc2-c2ccc(N(c3ccc4cc(-c5ccccc5)ccc4c3)c3ccc4oc5ccccc5c4c3-c3ccc4ccccc4c3)cc21]

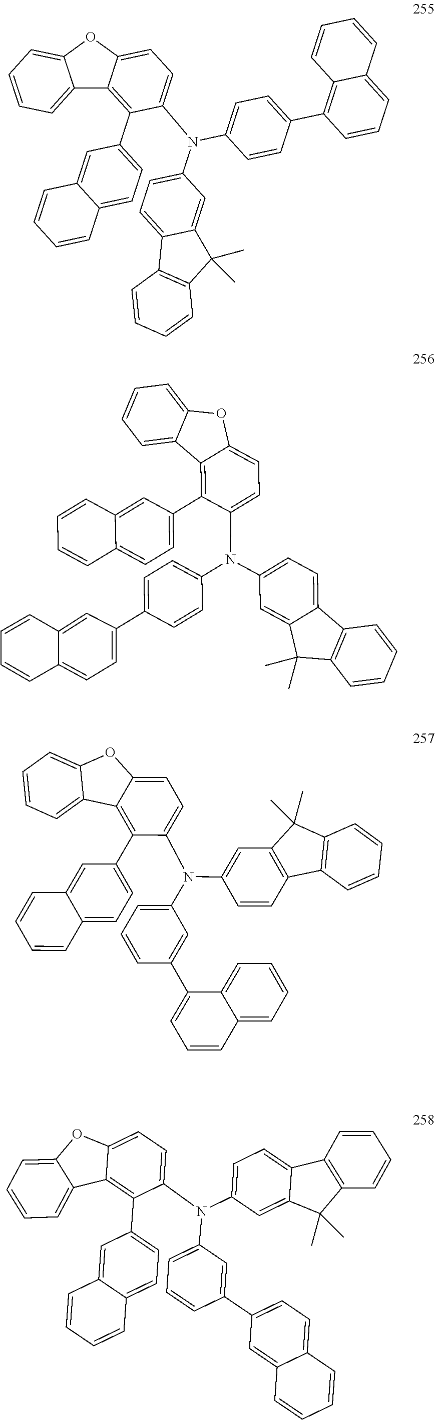 [SMILES: CC1(C)c2ccccc2-c2ccc(N(c3ccc(-c4cccc5ccccc45)cc3)c3ccc4oc5ccccc5c4c3-c3ccc4ccccc4c3)cc21 ; CC1(C)c2ccccc2-c2ccc(N(c3ccc(-c4ccc5ccccc5c4)cc3)c3ccc4oc5ccccc5c4c3-c3ccc4ccccc4c3)cc21 ; CC1(C)c2ccccc2-c2ccc(N(c3cccc(-c4cccc5ccccc45)c3)c3ccc4oc5ccccc5c4c3-c3ccc4ccccc4c3)cc21 ; CC1(C)c2ccccc2-c2ccc(N(c3cccc(-c4ccc5ccccc5c4)c3)c3ccc4oc5ccccc5c4c3-c3ccc4ccccc4c3)cc21]

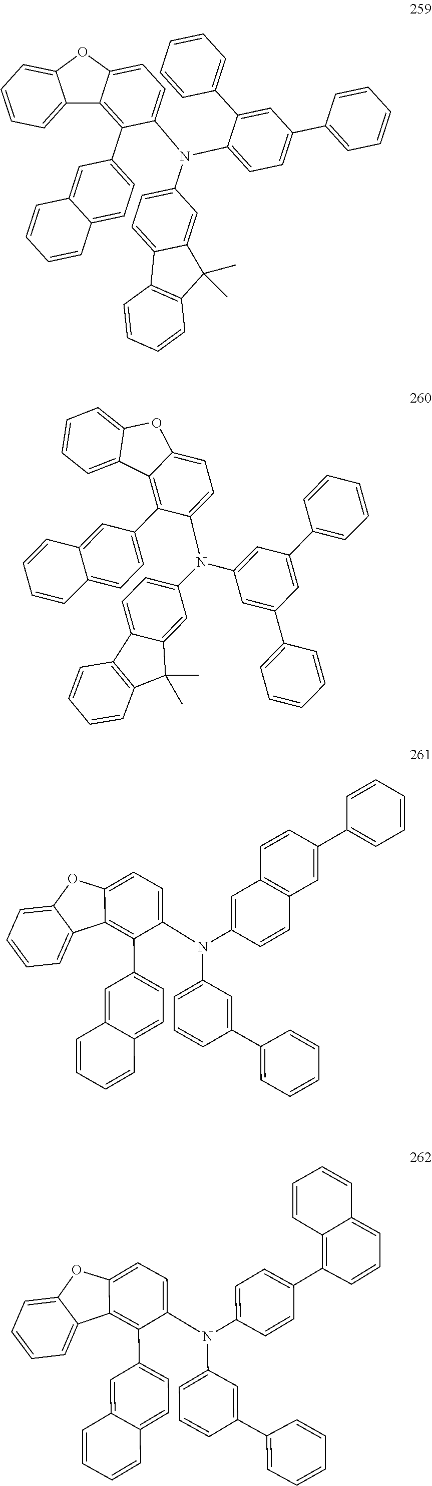 [SMILES: CC1(C)c2ccccc2-c2ccc(N(c3ccc(-c4ccccc4)cc3-c3ccccc3)c3ccc4oc5ccccc5c4c3-c3ccc4ccccc4c3)cc21 ; CC1(C)c2ccccc2-c2ccc(N(c3cc(-c4ccccc4)cc(-c4ccccc4)c3)c3ccc4oc5ccccc5c4c3-c3ccc4ccccc4c3)cc21 ; c1ccc(-c2cccc(N(c3ccc4cc(-c5ccccc5)ccc4c3)c3ccc4oc5ccccc5c4c3-c3ccc4ccccc4c3)c2)cc1 ; c1ccc(-c2cccc(N(c3ccc(-c4cccc5ccccc45)cc3)c3ccc4oc5ccccc5c4c3-c3ccc4ccccc4c3)c2)cc1]

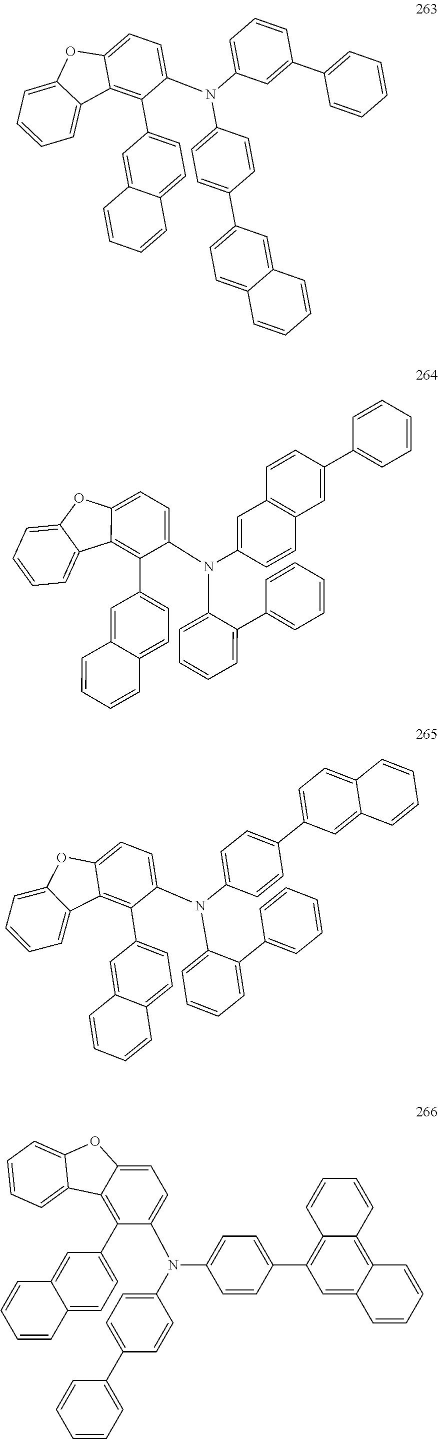 [SMILES: c1ccc(-c2cccc(N(c3ccc(-c4ccc5ccccc5c4)cc3)c3ccc4oc5ccccc5c4c3-c3ccc4ccccc4c3)c2)cc1 ; c1ccc(-c2ccc3cc(N(c4ccccc4-c4ccccc4)c4ccc5oc6ccccc6c5c4-c4ccc5ccccc5c4)ccc3c2)cc1 ; c1ccc(-c2ccccc2N(c2ccc(-c3ccc4ccccc4c3)cc2)c2ccc3oc4ccccc4c3c2-c2ccc3ccccc3c2)cc1 ; c1ccc(-c2ccc(N(c3ccc(-c4cc5ccccc5c5ccccc45)cc3)c3ccc4oc5ccccc5c4c3-c3ccc4ccccc4c3)cc2)cc1]

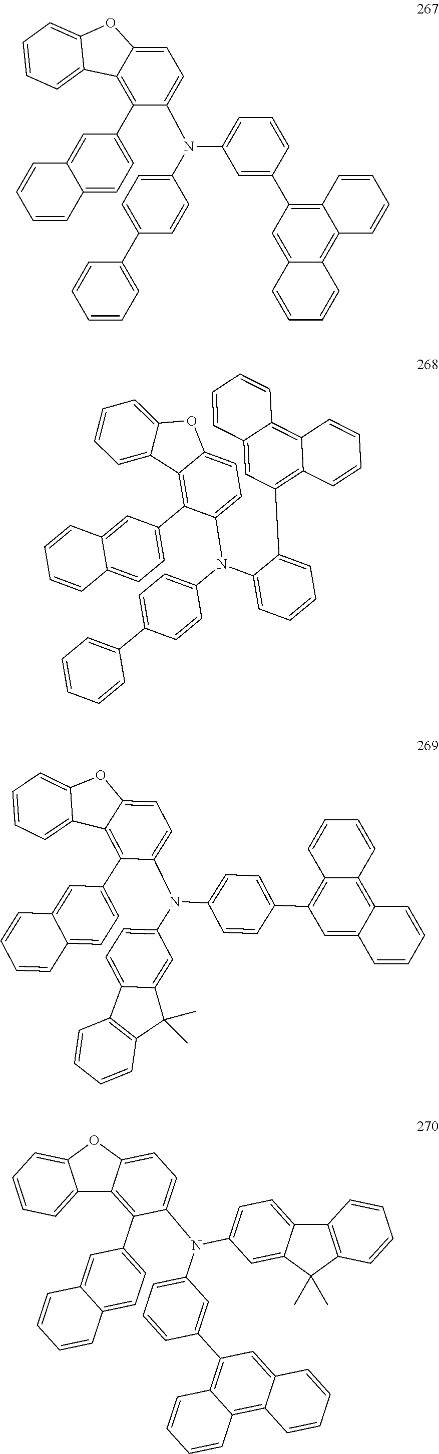 [SMILES: c1ccc(-c2ccc(N(c3cccc(-c4cc5ccccc5c5ccccc45)c3)c3ccc4oc5ccccc5c4c3-c3ccc4ccccc4c3)cc2)cc1 ; c1ccc(-c2ccc(N(c3ccccc3-c3cc4ccccc4c4ccccc34)c3ccc4oc5ccccc5c4c3-c3ccc4ccccc4c3)cc2)cc1 ; CC1(C)c2ccccc2-c2ccc(N(c3ccc(-c4cc5ccccc5c5ccccc45)cc3)c3ccc4oc5ccccc5c4c3-c3ccc4ccccc4c3)cc21 ; CC1(C)c2ccccc2-c2ccc(N(c3cccc(-c4cc5ccccc5c5ccccc45)c3)c3ccc4oc5ccccc5c4c3-c3ccc4ccccc4c3)cc21]

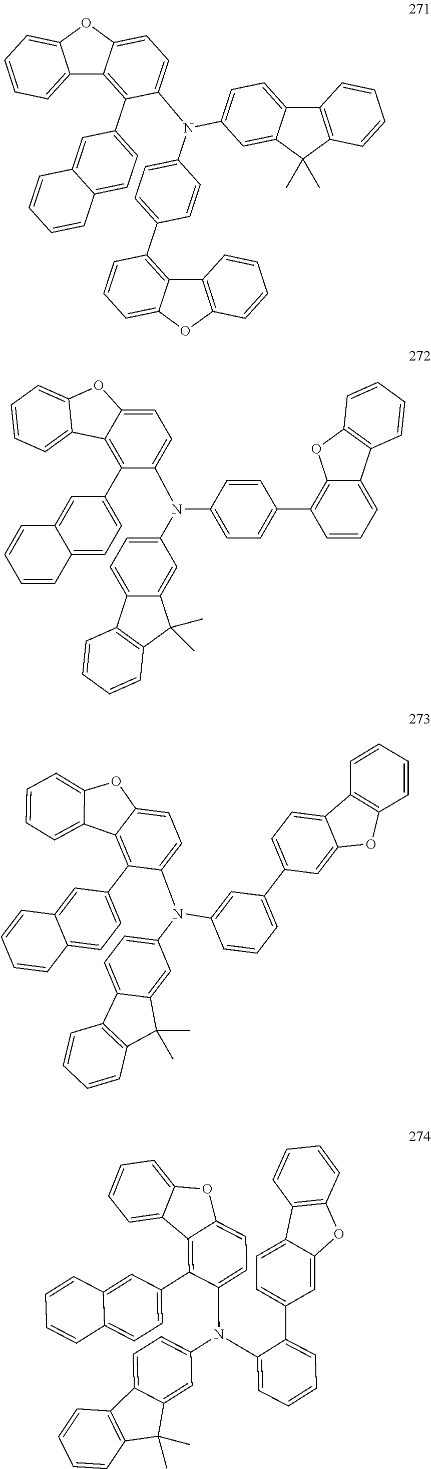 [SMILES: CC1(C)c2ccccc2-c2ccc(N(c3ccc(-c4cccc5oc6ccccc6c45)cc3)c3ccc4oc5ccccc5c4c3-c3ccc4ccccc4c3)cc21 ; CC1(C)c2ccccc2-c2ccc(N(c3ccc(-c4cccc5c4oc4ccccc45)cc3)c3ccc4oc5ccccc5c4c3-c3ccc4ccccc4c3)cc21 ; CC1(C)c2ccccc2-c2ccc(N(c3cccc(-c4ccc5c(c4)oc4ccccc45)c3)c3ccc4oc5ccccc5c4c3-c3ccc4ccccc4c3)cc21 ; CC1(C)c2ccccc2-c2ccc(N(c3ccccc3-c3ccc4c(c3)oc3ccccc34)c3ccc4oc5ccccc5c4c3-c3ccc4ccccc4c3)cc21]

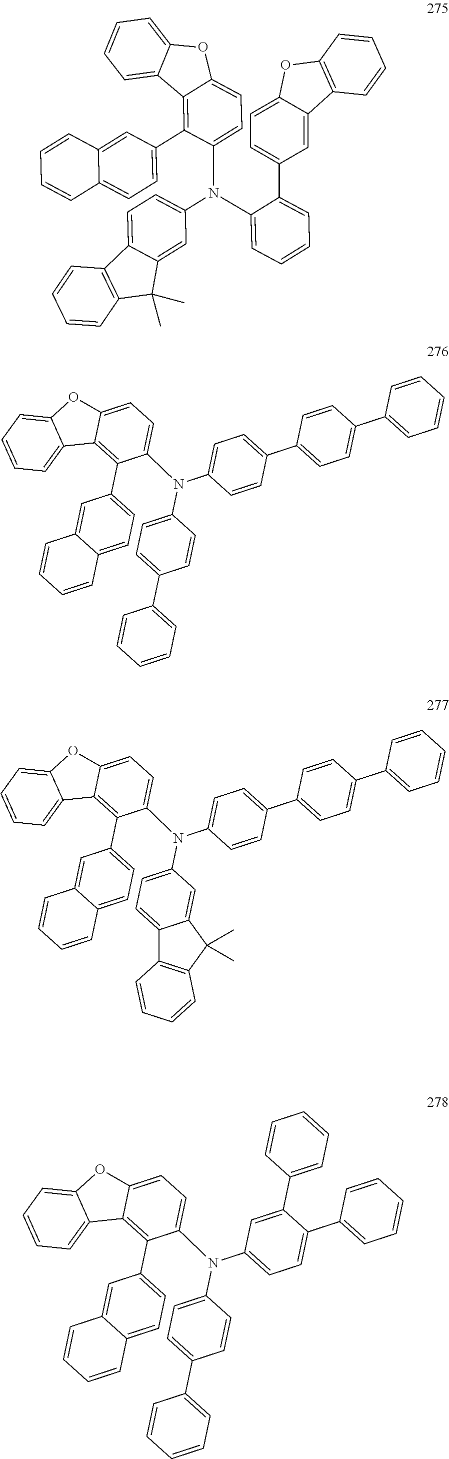 [SMILES: CC1(C)c2ccccc2-c2ccc(N(c3ccccc3-c3ccc4oc5ccccc5c4c3)c3ccc4oc5ccccc5c4c3-c3ccc4ccccc4c3)cc21 ; c1ccc(-c2ccc(-c3ccc(N(c4ccc(-c5ccccc5)cc4)c4ccc5oc6ccccc6c5c4-c4ccc5ccccc5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4ccccc4c3)cc21 ; c1ccc(-c2ccc(N(c3ccc(-c4ccccc4)c(-c4ccccc4)c3)c3ccc4oc5ccccc5c4c3-c3ccc4ccccc4c3)cc2)cc1]

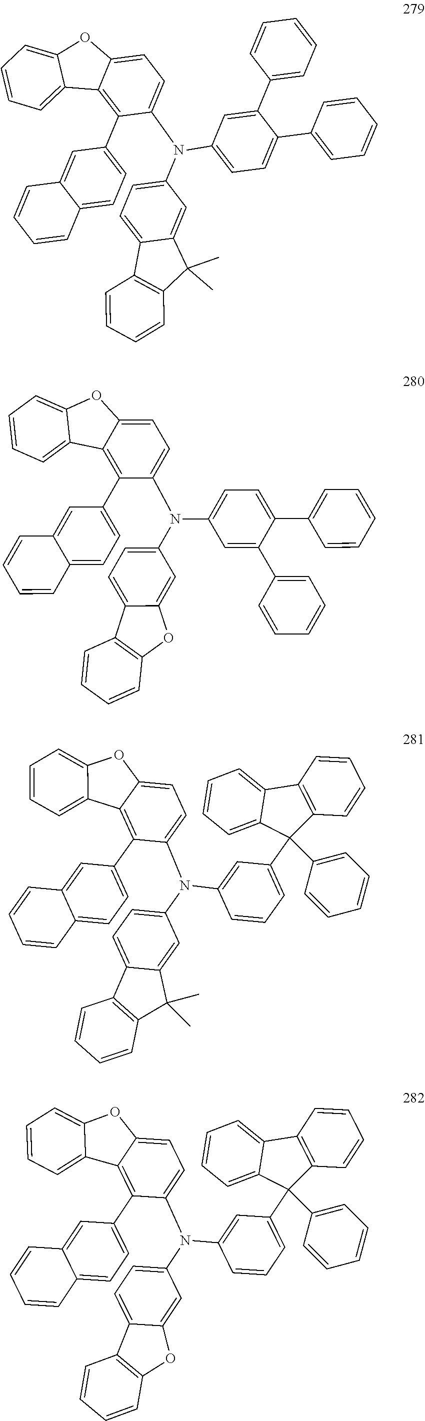 [SMILES: CC1(C)c2ccccc2-c2ccc(N(c3ccc(-c4ccccc4)c(-c4ccccc4)c3)c3ccc4oc5ccccc5c4c3-c3ccc4ccccc4c3)cc21 ; c1ccc(-c2ccc(N(c3ccc4c(c3)oc3ccccc34)c3ccc4oc5ccccc5c4c3-c3ccc4ccccc4c3)cc2-c2ccccc2)cc1 ; CC1(C)c2ccccc2-c2ccc(N(c3cccc(C4(c5ccccc5)c5ccccc5-c5ccccc54)c3)c3ccc4oc5ccccc5c4c3-c3ccc4ccccc4c3)cc21 ; c1ccc(C2(c3cccc(N(c4ccc5c(c4)oc4ccccc45)c4ccc5oc6ccccc6c5c4-c4ccc5ccccc5c4)c3)c3ccccc3-c3ccccc32)cc1]

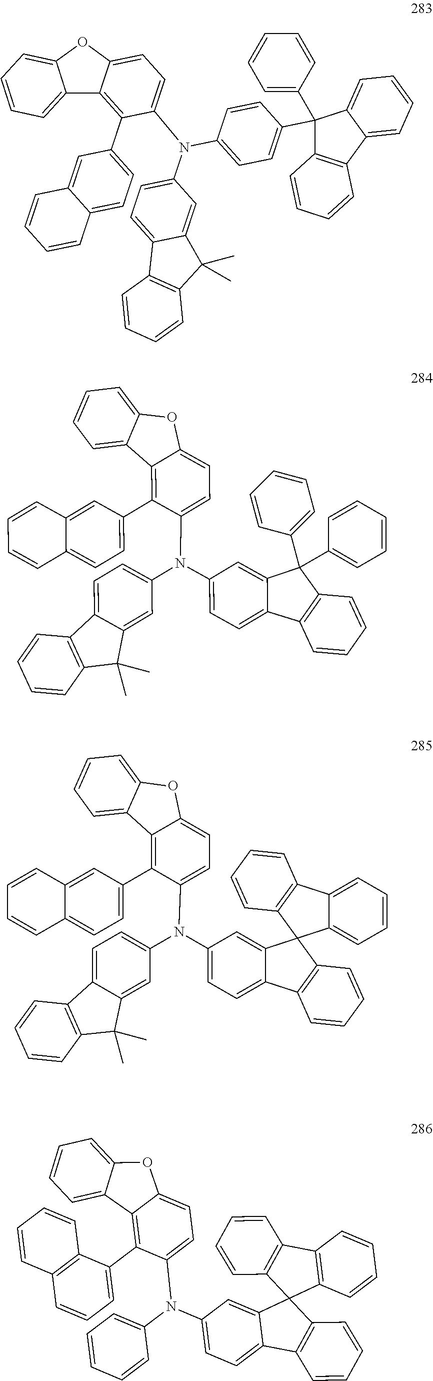 [SMILES: CC1(C)c2ccccc2-c2ccc(N(c3ccc(C4(c5ccccc5)c5ccccc5-c5ccccc54)cc3)c3ccc4oc5ccccc5c4c3-c3ccc4ccccc4c3)cc21 ; CC1(C)c2ccccc2-c2ccc(N(c3ccc4c(c3)C(c3ccccc3)(c3ccccc3)c3ccccc3-4)c3ccc4oc5ccccc5c4c3-c3ccc4ccccc4c3)cc21 ; CC1(C)c2ccccc2-c2ccc(N(c3ccc4c(c3)C3(c5ccccc5-c5ccccc53)c3ccccc3-4)c3ccc4oc5ccccc5c4c3-c3ccc4ccccc4c3)cc21 ; c1ccc(N(c2ccc3c(c2)C2(c4ccccc4-c4ccccc42)c2ccccc2-3)c2ccc3oc4ccccc4c3c2-c2cccc3ccccc23)cc1]

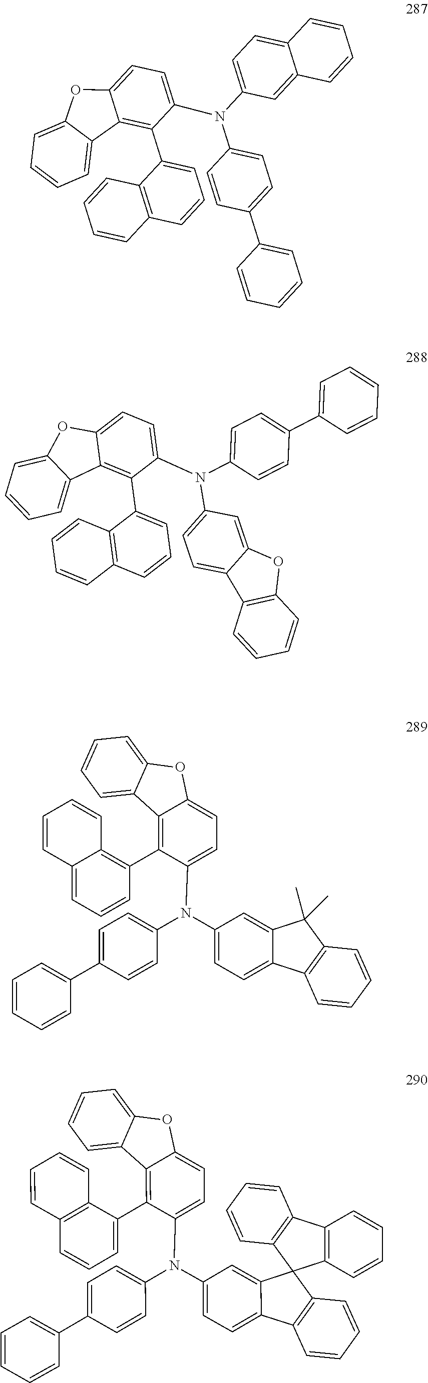 [SMILES: c1ccc(-c2ccc(N(c3ccc4ccccc4c3)c3ccc4oc5ccccc5c4c3-c3cccc4ccccc34)cc2)cc1 ; c1ccc(-c2ccc(N(c3ccc4c(c3)oc3ccccc34)c3ccc4oc5ccccc5c4c3-c3cccc4ccccc34)cc2)cc1 ; CC1(C)c2ccccc2-c2ccc(N(c3ccc(-c4ccccc4)cc3)c3ccc4oc5ccccc5c4c3-c3cccc4ccccc34)cc21 ; c1ccc(-c2ccc(N(c3ccc4c(c3)C3(c5ccccc5-c5ccccc53)c3ccccc3-4)c3ccc4oc5ccccc5c4c3-c3cccc4ccccc34)cc2)cc1]

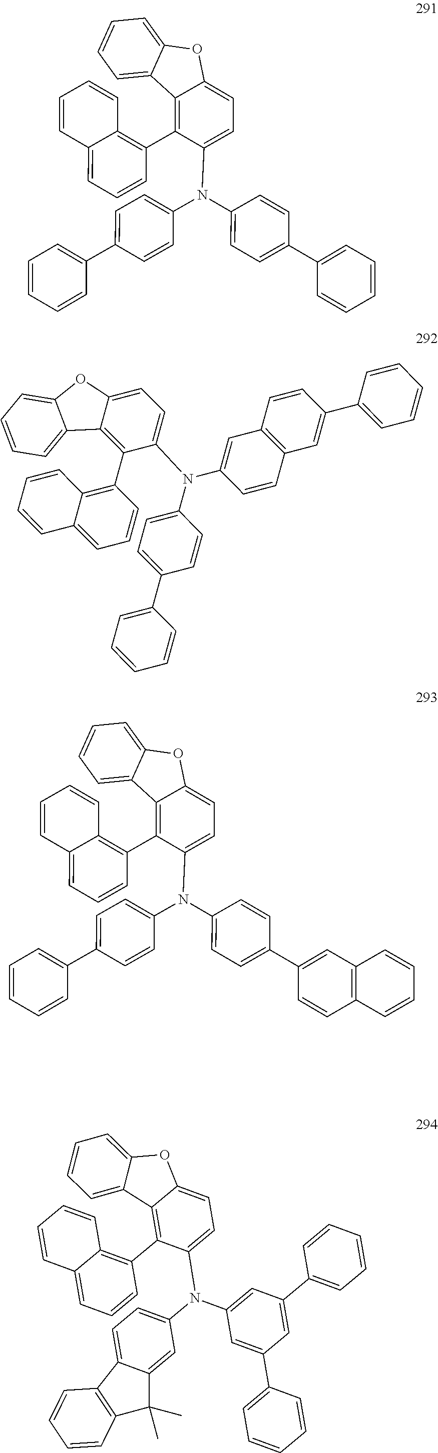 [SMILES: c1ccc(-c2ccc(N(c3ccc(-c4ccccc4)cc3)c3ccc4oc5ccccc5c4c3-c3cccc4ccccc34)cc2)cc1 ; c1ccc(-c2ccc(N(c3ccc4cc(-c5ccccc5)ccc4c3)c3ccc4oc5ccccc5c4c3-c3cccc4ccccc34)cc2)cc1 ; c1ccc(-c2ccc(N(c3ccc(-c4ccc5ccccc5c4)cc3)c3ccc4oc5ccccc5c4c3-c3cccc4ccccc34)cc2)cc1 ; CC1(C)c2ccccc2-c2ccc(N(c3cc(-c4ccccc4)cc(-c4ccccc4)c3)c3ccc4oc5ccccc5c4c3-c3cccc4ccccc34)cc21]

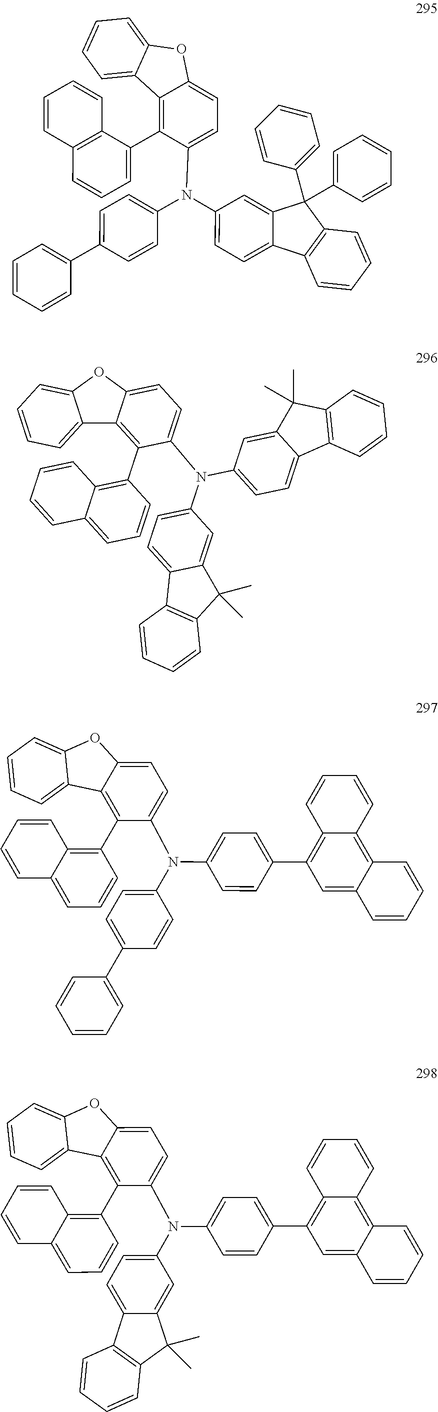 [SMILES: c1ccc(-c2ccc(N(c3ccc4c(c3)C(c3ccccc3)(c3ccccc3)c3ccccc3-4)c3ccc4oc5ccccc5c4c3-c3cccc4ccccc34)cc2)cc1 ; CC1(C)c2ccccc2-c2ccc(N(c3ccc4c(c3)C(C)(C)c3ccccc3-4)c3ccc4oc5ccccc5c4c3-c3cccc4ccccc34)cc21 ; c1ccc(-c2ccc(N(c3ccc(-c4cc5ccccc5c5ccccc45)cc3)c3ccc4oc5ccccc5c4c3-c3cccc4ccccc34)cc2)cc1 ; CC1(C)c2ccccc2-c2ccc(N(c3ccc(-c4cc5ccccc5c5ccccc45)cc3)c3ccc4oc5ccccc5c4c3-c3cccc4ccccc34)cc21]

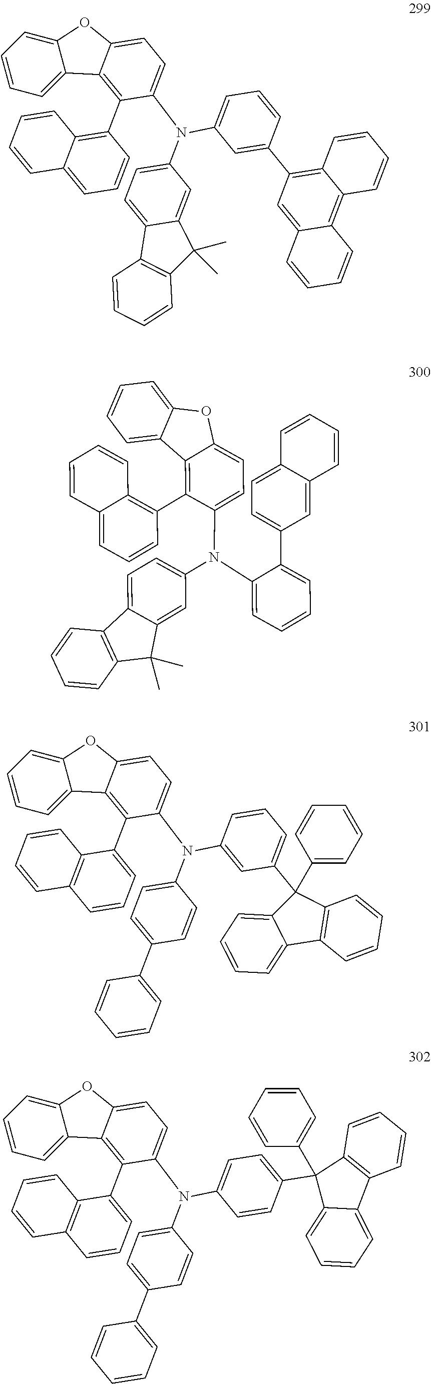 [SMILES: CC1(C)c2ccccc2-c2ccc(N(c3cccc(-c4cc5ccccc5c5ccccc45)c3)c3ccc4oc5ccccc5c4c3-c3cccc4ccccc34)cc21 ; CC1(C)c2ccccc2-c2ccc(N(c3ccccc3-c3ccc4ccccc4c3)c3ccc4oc5ccccc5c4c3-c3cccc4ccccc34)cc21 ; c1ccc(-c2ccc(N(c3cccc(C4(c5ccccc5)c5ccccc5-c5ccccc54)c3)c3ccc4oc5ccccc5c4c3-c3cccc4ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cccc4ccccc34)cc2)cc1]

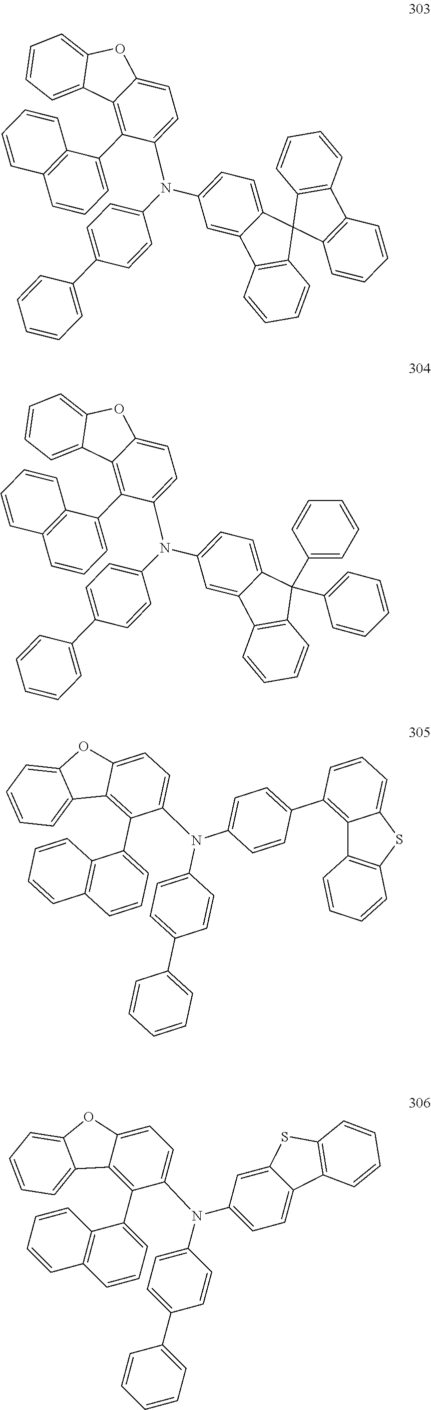 [SMILES: c1ccc(-c2ccc(N(c3ccc4c(c3)-c3ccccc3C43c4ccccc4-c4ccccc43)c3ccc4oc5ccccc5c4c3-c3cccc4ccccc34)cc2)cc1 ; c1ccc(-c2ccc(N(c3ccc4c(c3)-c3ccccc3C4(c3ccccc3)c3ccccc3)c3ccc4oc5ccccc5c4c3-c3cccc4ccccc34)cc2)cc1 ; c1ccc(-c2ccc(N(c3ccc(-c4cccc5sc6ccccc6c45)cc3)c3ccc4oc5ccccc5c4c3-c3cccc4ccccc34)cc2)cc1 ; c1ccc(-c2ccc(N(c3ccc4c(c3)sc3ccccc34)c3ccc4oc5ccccc5c4c3-c3cccc4ccccc34)cc2)cc1]

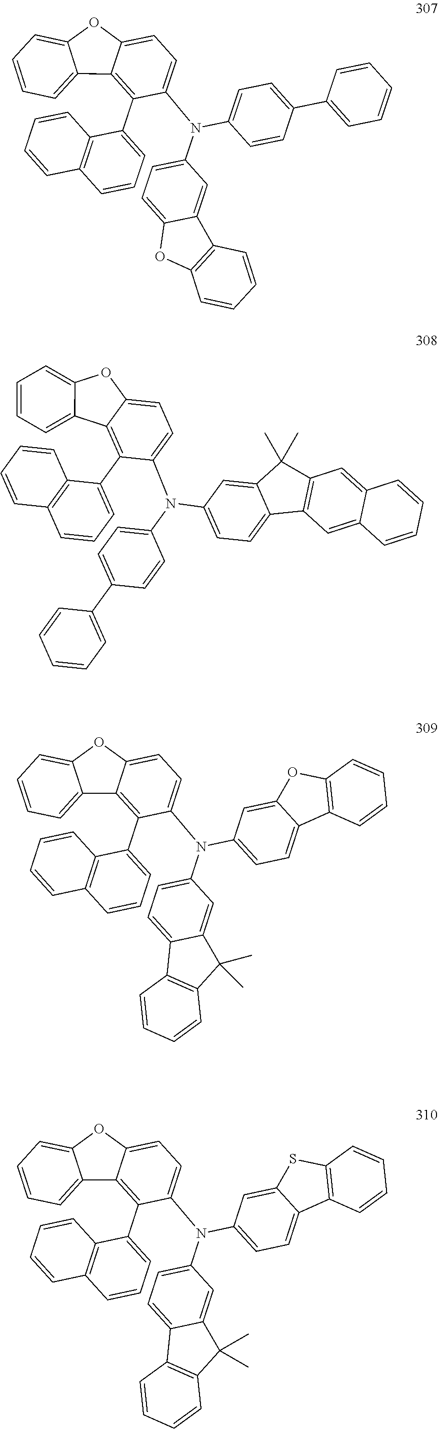 [SMILES: c1ccc(-c2ccc(N(c3ccc4oc5ccccc5c4c3)c3ccc4oc5ccccc5c4c3-c3cccc4ccccc34)cc2)cc1 ; CC1(C)c2cc(N(c3ccc(-c4ccccc4)cc3)c3ccc4oc5ccccc5c4c3-c3cccc4ccccc34)ccc2-c2cc3ccccc3cc21 ; CC1(C)c2ccccc2-c2ccc(N(c3ccc4c(c3)oc3ccccc34)c3ccc4oc5ccccc5c4c3-c3cccc4ccccc34)cc21 ; CC1(C)c2ccccc2-c2ccc(N(c3ccc4c(c3)sc3ccccc34)c3ccc4oc5ccccc5c4c3-c3cccc4ccccc34)cc21]

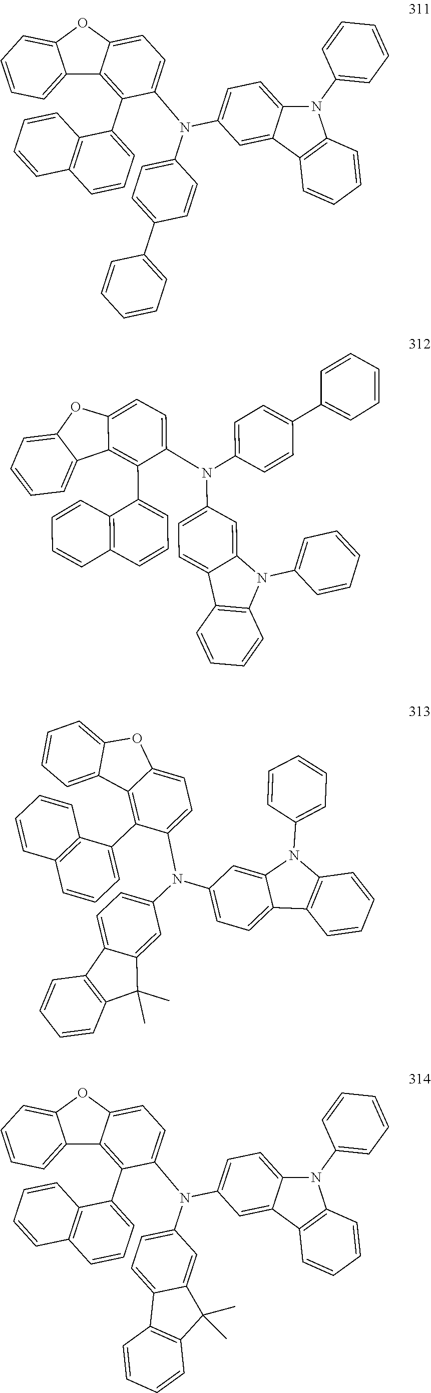 [SMILES: c1ccc(-c2ccc(N(c3ccc4c(c3)c3ccccc3n4-c3ccccc3)c3ccc4oc5ccccc5c4c3-c3cccc4ccccc34)cc2)cc1 ; c1ccc(-c2ccc(N(c3ccc4c5ccccc5n(-c5ccccc5)c4c3)c3ccc4oc5ccccc5c4c3-c3cccc4ccccc34)cc2)cc1 ; CC1(C)c2ccccc2-c2ccc(N(c3ccc4c5ccccc5n(-c5ccccc5)c4c3)c3ccc4oc5ccccc5c4c3-c3cccc4ccccc34)cc21 ; CC1(C)c2ccccc2-c2ccc(N(c3ccc4c(c3)c3ccccc3n4-c3ccccc3)c3ccc4oc5ccccc5c4c3-c3cccc4ccccc34)cc21]

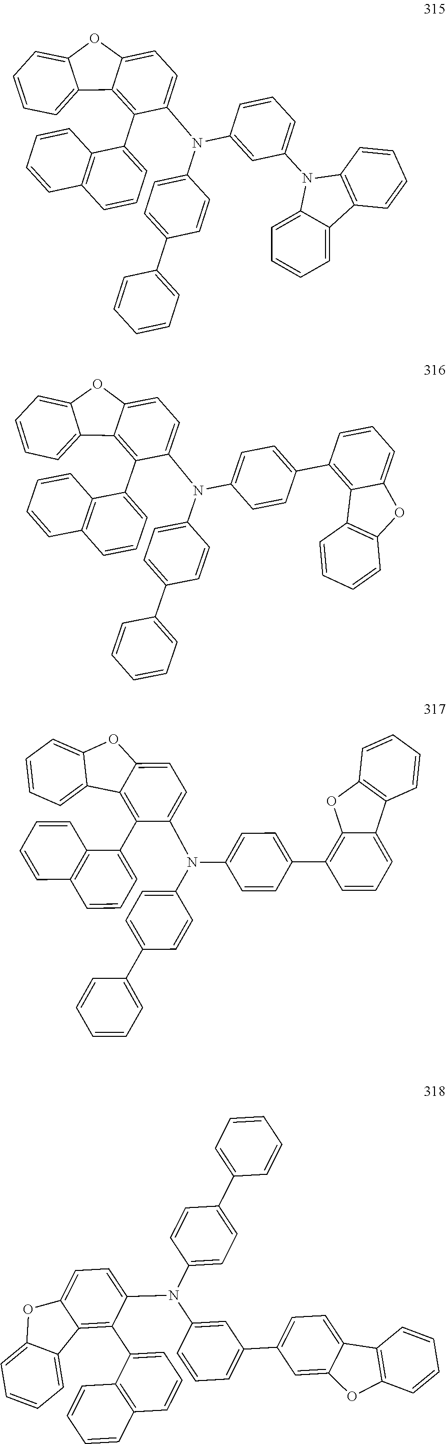 [SMILES: c1ccc(-c2ccc(N(c3cccc(-n4c5ccccc5c5ccccc54)c3)c3ccc4oc5ccccc5c4c3-c3cccc4ccccc34)cc2)cc1 ; c1ccc(-c2ccc(N(c3ccc(-c4cccc5oc6ccccc6c45)cc3)c3ccc4oc5ccccc5c4c3-c3cccc4ccccc34)cc2)cc1 ; c1ccc(-c2ccc(N(c3ccc(-c4cccc5c4oc4ccccc45)cc3)c3ccc4oc5ccccc5c4c3-c3cccc4ccccc34)cc2)cc1 ; c1ccc(-c2ccc(N(c3cccc(-c4ccc5c(c4)oc4ccccc45)c3)c3ccc4oc5ccccc5c4c3-c3cccc4ccccc34)cc2)cc1]

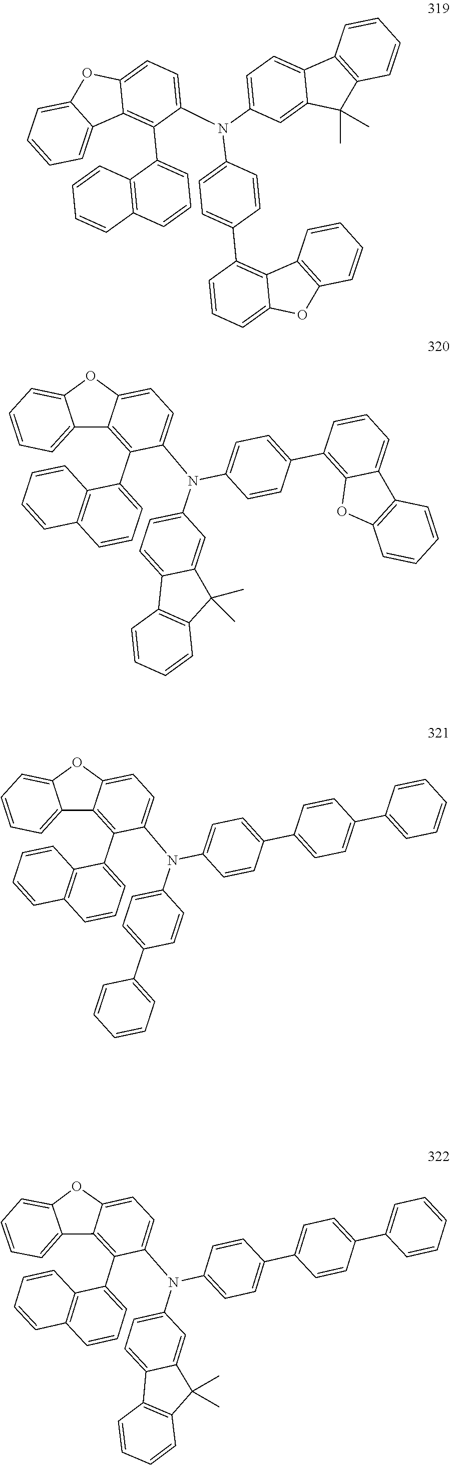 [SMILES: CC1(C)c2ccccc2-c2ccc(N(c3ccc(-c4cccc5oc6ccccc6c45)cc3)c3ccc4oc5ccccc5c4c3-c3cccc4ccccc34)cc21 ; CC1(C)c2ccccc2-c2ccc(N(c3ccc(-c4cccc5c4oc4ccccc45)cc3)c3ccc4oc5ccccc5c4c3-c3cccc4ccccc34)cc21 ; c1ccc(-c2ccc(-c3ccc(N(c4ccc(-c5ccccc5)cc4)c4ccc5oc6ccccc6c5c4-c4cccc5ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cccc4ccccc34)cc21]

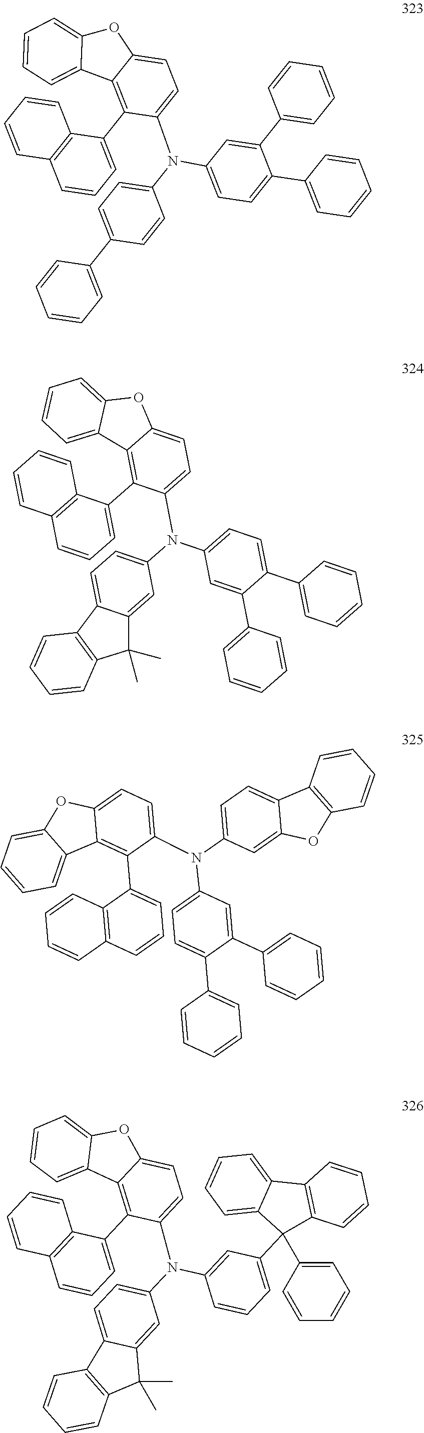 [SMILES: c1ccc(-c2ccc(N(c3ccc(-c4ccccc4)c(-c4ccccc4)c3)c3ccc4oc5ccccc5c4c3-c3cccc4ccccc34)cc2)cc1 ; CC1(C)c2ccccc2-c2ccc(N(c3ccc(-c4ccccc4)c(-c4ccccc4)c3)c3ccc4oc5ccccc5c4c3-c3cccc4ccccc34)cc21 ; c1ccc(-c2ccc(N(c3ccc4c(c3)oc3ccccc34)c3ccc4oc5ccccc5c4c3-c3cccc4ccccc34)cc2-c2ccccc2)cc1 ; CC1(C)c2ccccc2-c2ccc(N(c3cccc(C4(c5ccccc5)c5ccccc5-c5ccccc54)c3)c3ccc4oc5ccccc5c4c3-c3cccc4ccccc34)cc21]

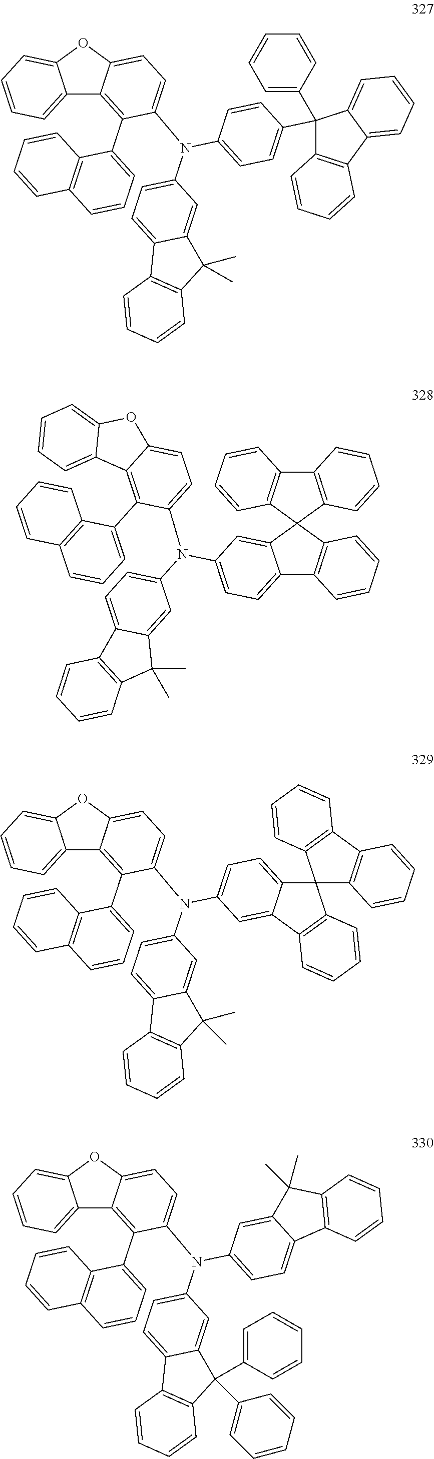 [SMILES: CC1(C)c2ccccc2-c2ccc(N(c3ccc(C4(c5ccccc5)c5ccccc5-c5ccccc54)cc3)c3ccc4oc5ccccc5c4c3-c3cccc4ccccc34)cc21 ; CC1(C)c2ccccc2-c2ccc(N(c3ccc4c(c3)C3(c5ccccc5-c5ccccc53)c3ccccc3-4)c3ccc4oc5ccccc5c4c3-c3cccc4ccccc34)cc21 ; CC1(C)c2ccccc2-c2ccc(N(c3ccc4c(c3)-c3ccccc3C43c4ccccc4-c4ccccc43)c3ccc4oc5ccccc5c4c3-c3cccc4ccccc34)cc21 ; CC1(C)c2ccccc2-c2ccc(N(c3ccc4c(c3)C(c3ccccc3)(c3ccccc3)c3ccccc3-4)c3ccc4oc5ccccc5c4c3-c3cccc4ccccc34)cc21]

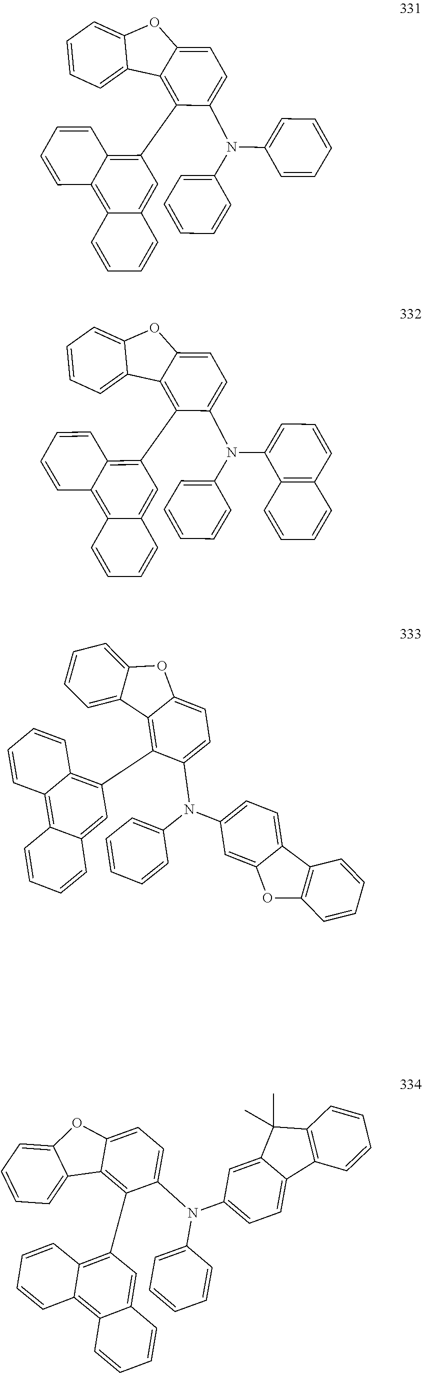 [SMILES: c1ccc(N(c2ccccc2)c2ccc3oc4ccccc4c3c2-c2cc3ccccc3c3ccccc23)cc1 ; c1ccc(N(c2ccc3oc4ccccc4c3c2-c2cc3ccccc3c3ccccc23)c2cccc3ccccc23)cc1 ; c1ccc(N(c2ccc3c(c2)oc2ccccc23)c2ccc3oc4ccccc4c3c2-c2cc3ccccc3c3ccccc23)cc1 ; CC1(C)c2ccccc2-c2ccc(N(c3ccccc3)c3ccc4oc5ccccc5c4c3-c3cc4ccccc4c4ccccc34)cc21]

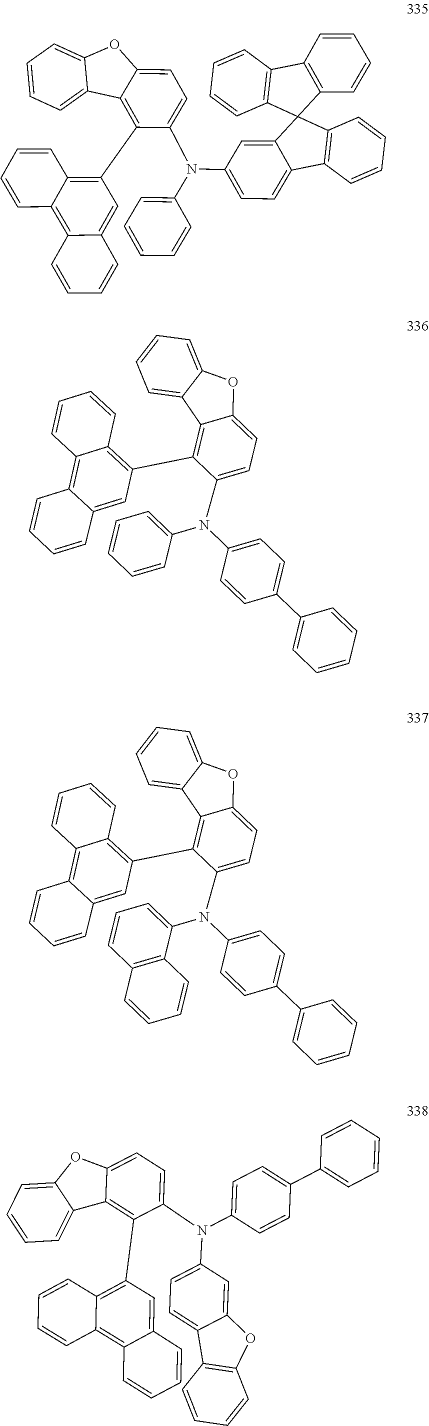 [SMILES: c1ccc(N(c2ccc3c(c2)C2(c4ccccc4-c4ccccc42)c2ccccc2-3)c2ccc3oc4ccccc4c3c2-c2cc3ccccc3c3ccccc23)cc1 ; c1ccc(-c2ccc(N(c3ccccc3)c3ccc4oc5ccccc5c4c3-c3cc4ccccc4c4ccccc34)cc2)cc1 ; c1ccc(-c2ccc(N(c3ccc4oc5ccccc5c4c3-c3cc4ccccc4c4ccccc34)c3cccc4ccccc34)cc2)cc1 ; c1ccc(-c2ccc(N(c3ccc4c(c3)oc3ccccc34)c3ccc4oc5ccccc5c4c3-c3cc4ccccc4c4ccccc34)cc2)cc1]

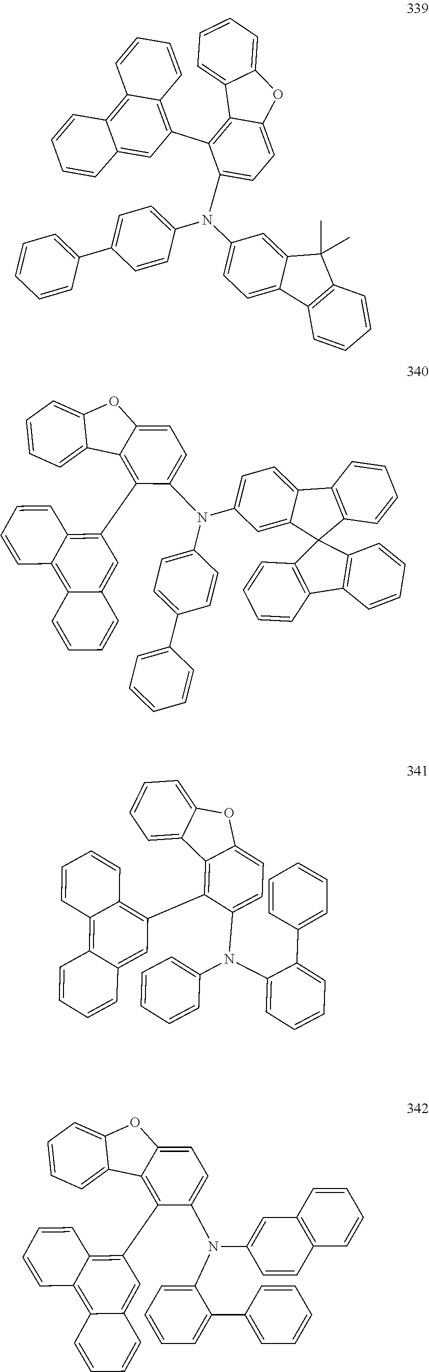 [SMILES: CC1(C)c2ccccc2-c2ccc(N(c3ccc(-c4ccccc4)cc3)c3ccc4oc5ccccc5c4c3-c3cc4ccccc4c4ccccc34)cc21 ; c1ccc(-c2ccc(N(c3ccc4c(c3)C3(c5ccccc5-c5ccccc53)c3ccccc3-4)c3ccc4oc5ccccc5c4c3-c3cc4ccccc4c4ccccc34)cc2)cc1 ; c1ccc(-c2ccccc2N(c2ccccc2)c2ccc3oc4ccccc4c3c2-c2cc3ccccc3c3ccccc23)cc1 ; c1ccc(-c2ccccc2N(c2ccc3ccccc3c2)c2ccc3oc4ccccc4c3c2-c2cc3ccccc3c3ccccc23)cc1]

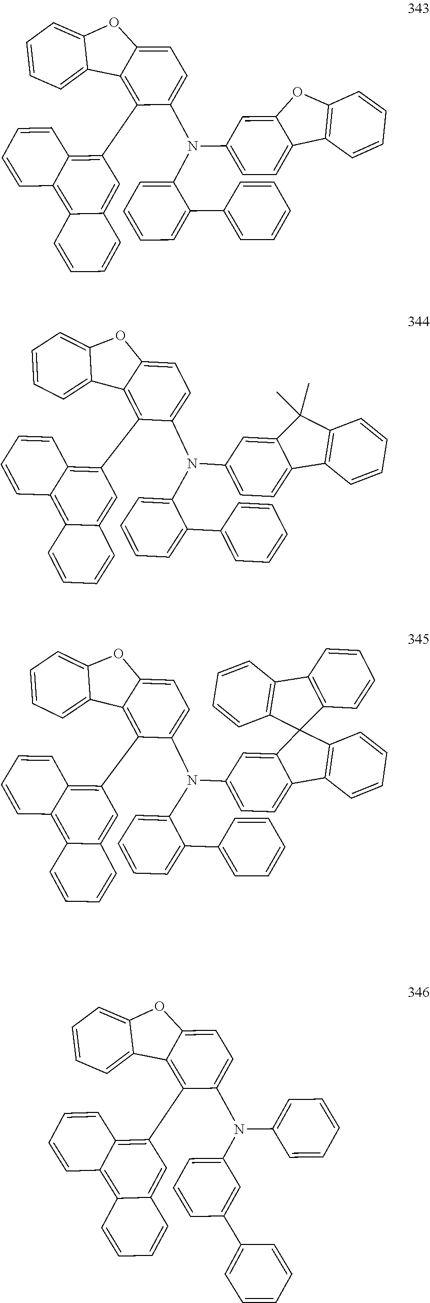 [SMILES: c1ccc(-c2ccccc2N(c2ccc3c(c2)oc2ccccc23)c2ccc3oc4ccccc4c3c2-c2cc3ccccc3c3ccccc23)cc1 ; CC1(C)c2ccccc2-c2ccc(N(c3ccccc3-c3ccccc3)c3ccc4oc5ccccc5c4c3-c3cc4ccccc4c4ccccc34)cc21 ; c1ccc(-c2ccccc2N(c2ccc3c(c2)C2(c4ccccc4-c4ccccc42)c2ccccc2-3)c2ccc3oc4ccccc4c3c2-c2cc3ccccc3c3ccccc23)cc1 ; c1ccc(-c2cccc(N(c3ccccc3)c3ccc4oc5ccccc5c4c3-c3cc4ccccc4c4ccccc34)c2)cc1]

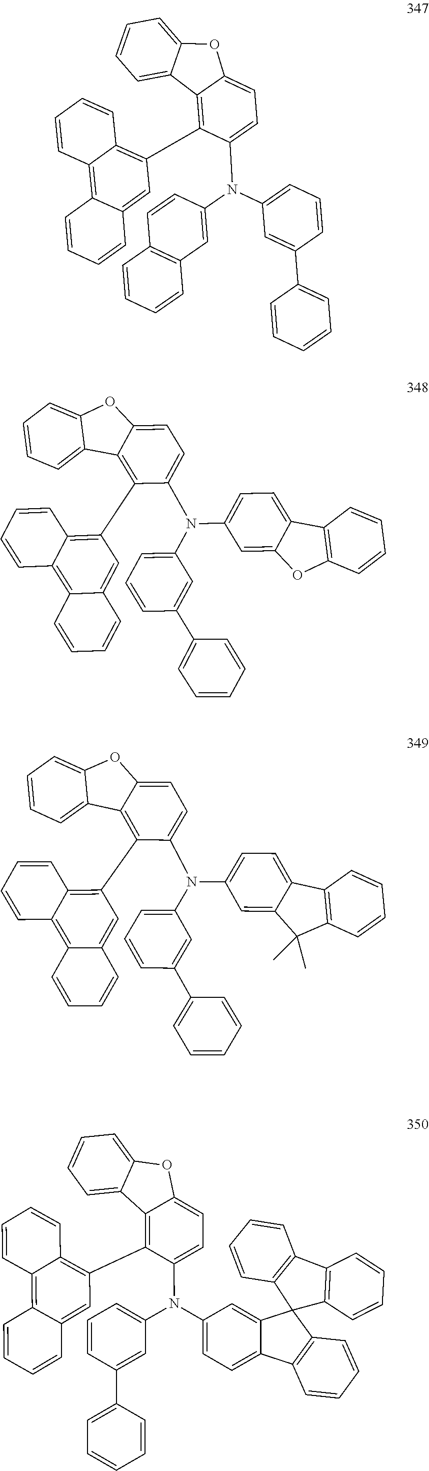 [SMILES: c1ccc(-c2cccc(N(c3ccc4ccccc4c3)c3ccc4oc5ccccc5c4c3-c3cc4ccccc4c4ccccc34)c2)cc1 ; c1ccc(-c2cccc(N(c3ccc4c(c3)oc3ccccc34)c3ccc4oc5ccccc5c4c3-c3cc4ccccc4c4ccccc34)c2)cc1 ; CC1(C)c2ccccc2-c2ccc(N(c3cccc(-c4ccccc4)c3)c3ccc4oc5ccccc5c4c3-c3cc4ccccc4c4ccccc34)cc21 ; c1ccc(-c2cccc(N(c3ccc4c(c3)C3(c5ccccc5-c5ccccc53)c3ccccc3-4)c3ccc4oc5ccccc5c4c3-c3cc4ccccc4c4ccccc34)c2)cc1]

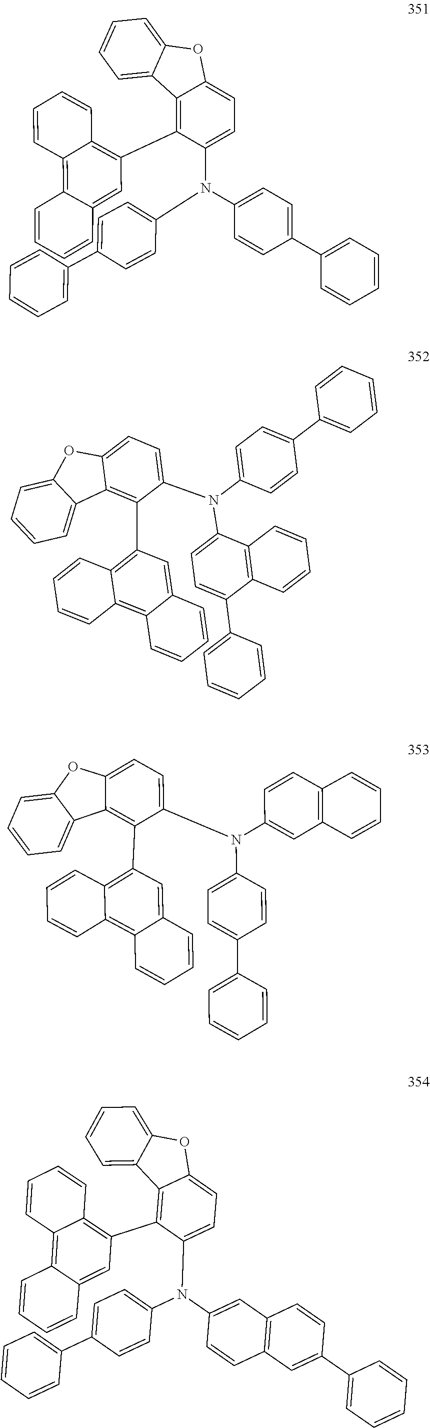 [SMILES: c1ccc(-c2ccc(N(c3ccc(-c4ccccc4)cc3)c3ccc4oc5ccccc5c4c3-c3cc4ccccc4c4ccccc34)cc2)cc1 ; c1ccc(-c2ccc(N(c3ccc4oc5ccccc5c4c3-c3cc4ccccc4c4ccccc34)c3ccc(-c4ccccc4)c4ccccc34)cc2)cc1 ; c1ccc(-c2ccc(N(c3ccc4ccccc4c3)c3ccc4oc5ccccc5c4c3-c3cc4ccccc4c4ccccc34)cc2)cc1 ; c1ccc(-c2ccc(N(c3ccc4cc(-c5ccccc5)ccc4c3)c3ccc4oc5ccccc5c4c3-c3cc4ccccc4c4ccccc34)cc2)cc1]

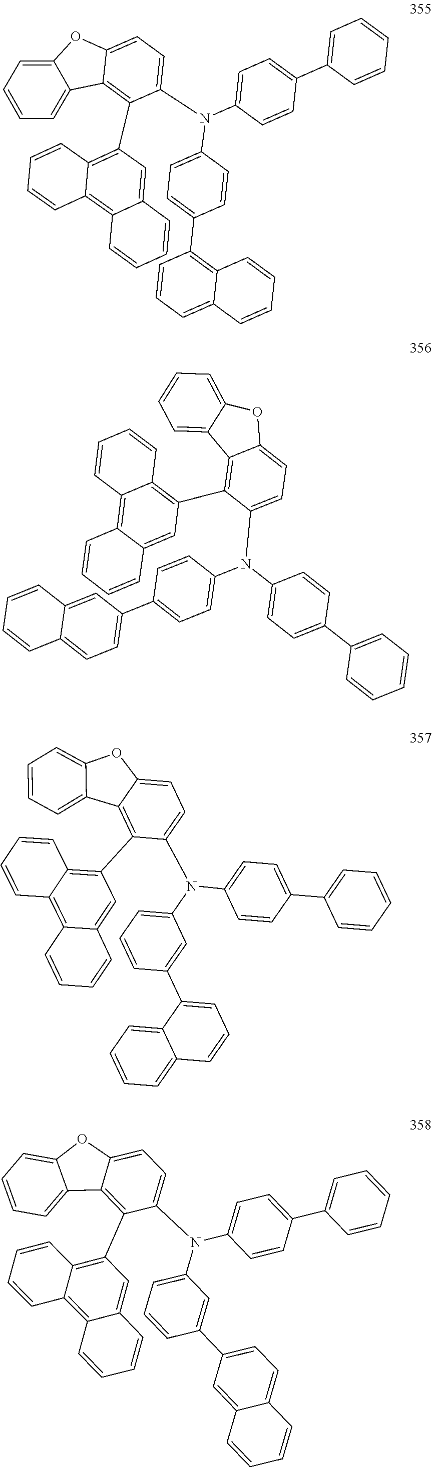 [SMILES: c1ccc(-c2ccc(N(c3ccc(-c4cccc5ccccc45)cc3)c3ccc4oc5ccccc5c4c3-c3cc4ccccc4c4ccccc34)cc2)cc1 ; c1ccc(-c2ccc(N(c3ccc(-c4ccc5ccccc5c4)cc3)c3ccc4oc5ccccc5c4c3-c3cc4ccccc4c4ccccc34)cc2)cc1 ; c1ccc(-c2ccc(N(c3cccc(-c4cccc5ccccc45)c3)c3ccc4oc5ccccc5c4c3-c3cc4ccccc4c4ccccc34)cc2)cc1 ; c1ccc(-c2ccc(N(c3cccc(-c4ccc5ccccc5c4)c3)c3ccc4oc5ccccc5c4c3-c3cc4ccccc4c4ccccc34)cc2)cc1]

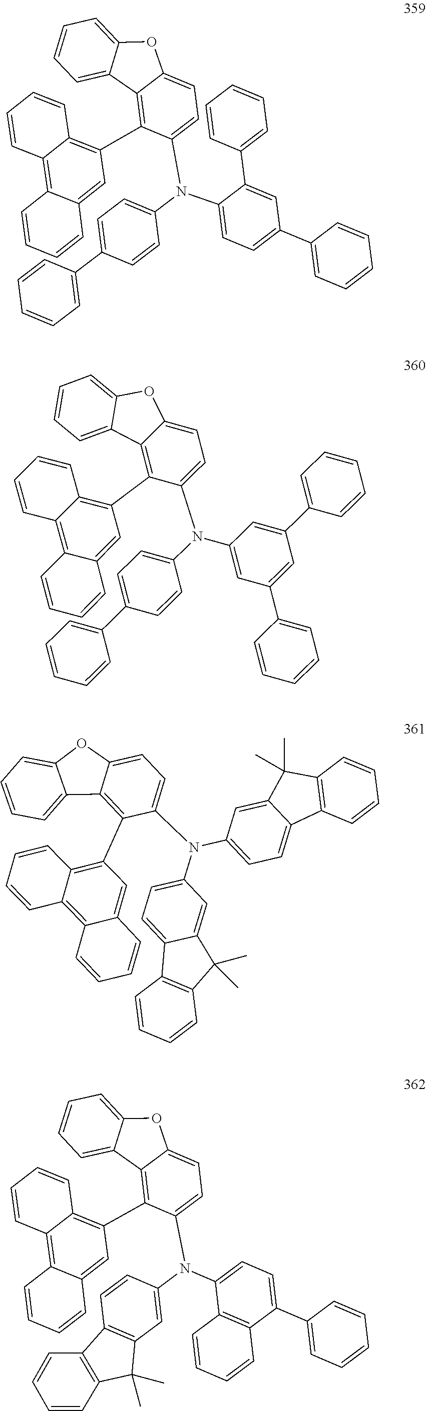 [SMILES: c1ccc(-c2ccc(N(c3ccc(-c4ccccc4)cc3-c3ccccc3)c3ccc4oc5ccccc5c4c3-c3cc4ccccc4c4ccccc34)cc2)cc1 ; c1ccc(-c2ccc(N(c3cc(-c4ccccc4)cc(-c4ccccc4)c3)c3ccc4oc5ccccc5c4c3-c3cc4ccccc4c4ccccc34)cc2)cc1 ; CC1(C)c2ccccc2-c2ccc(N(c3ccc4c(c3)C(C)(C)c3ccccc3-4)c3ccc4oc5ccccc5c4c3-c3cc4ccccc4c4ccccc34)cc21 ; CC1(C)c2ccccc2-c2ccc(N(c3ccc4oc5ccccc5c4c3-c3cc4ccccc4c4ccccc34)c3ccc(-c4ccccc4)c4ccccc34)cc21]

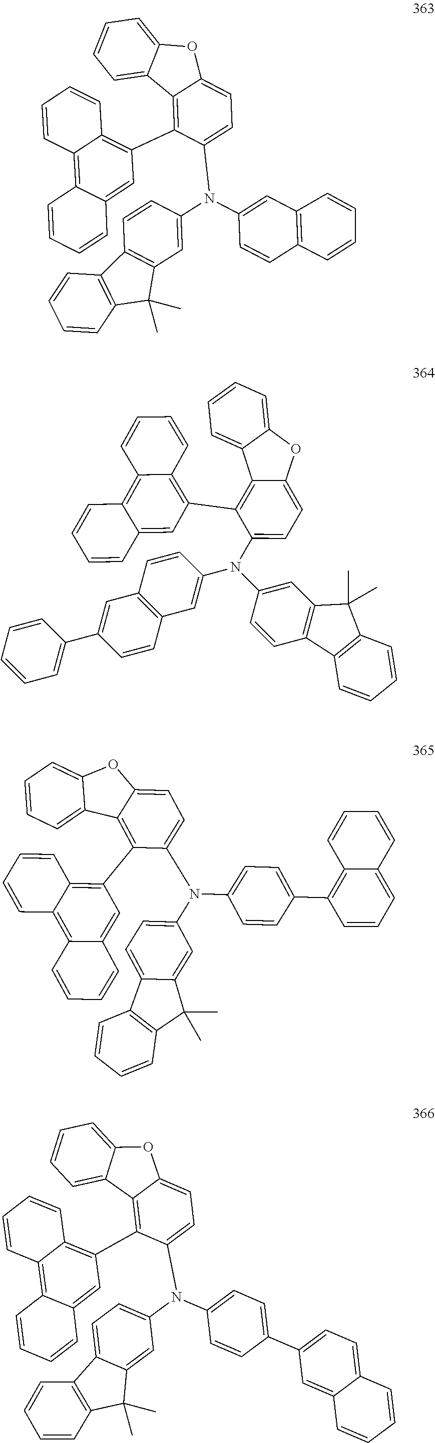 [SMILES: CC1(C)c2ccccc2-c2ccc(N(c3ccc4ccccc4c3)c3ccc4oc5ccccc5c4c3-c3cc4ccccc4c4ccccc34)cc21 ; CC1(C)c2ccccc2-c2ccc(N(c3ccc4cc(-c5ccccc5)ccc4c3)c3ccc4oc5ccccc5c4c3-c3cc4ccccc4c4ccccc34)cc21 ; CC1(C)c2ccccc2-c2ccc(N(c3ccc(-c4cccc5ccccc45)cc3)c3ccc4oc5ccccc5c4c3-c3cc4ccccc4c4ccccc34)cc21 ; CC1(C)c2ccccc2-c2ccc(N(c3ccc(-c4ccc5ccccc5c4)cc3)c3ccc4oc5ccccc5c4c3-c3cc4ccccc4c4ccccc34)cc21]

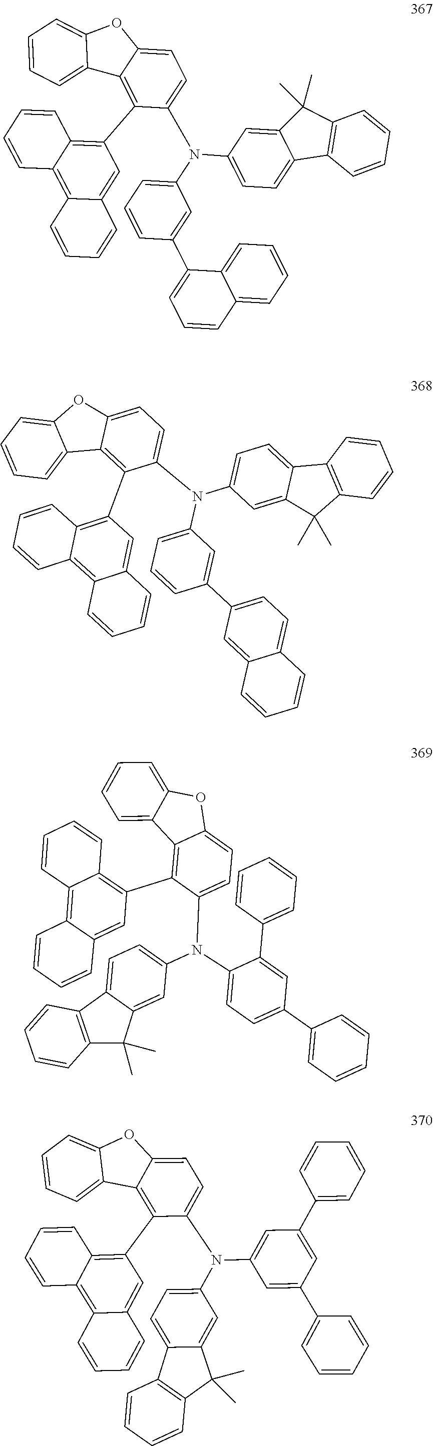 [SMILES: CC1(C)c2ccccc2-c2ccc(N(c3cccc(-c4cccc5ccccc45)c3)c3ccc4oc5ccccc5c4c3-c3cc4ccccc4c4ccccc34)cc21 ; CC1(C)c2ccccc2-c2ccc(N(c3cccc(-c4ccc5ccccc5c4)c3)c3ccc4oc5ccccc5c4c3-c3cc4ccccc4c4ccccc34)cc21 ; CC1(C)c2ccccc2-c2ccc(N(c3ccc(-c4ccccc4)cc3-c3ccccc3)c3ccc4oc5ccccc5c4c3-c3cc4ccccc4c4ccccc34)cc21 ; CC1(C)c2ccccc2-c2ccc(N(c3cc(-c4ccccc4)cc(-c4ccccc4)c3)c3ccc4oc5ccccc5c4c3-c3cc4ccccc4c4ccccc34)cc21]

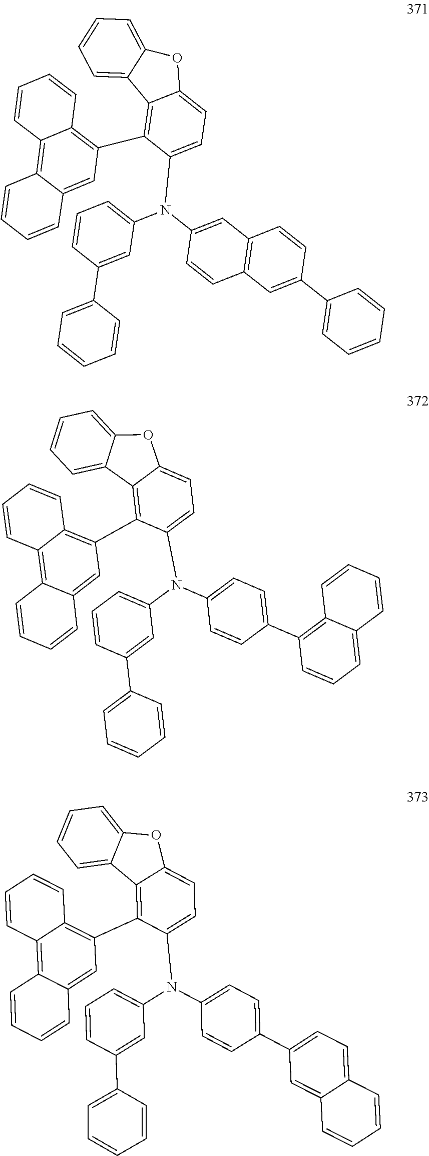

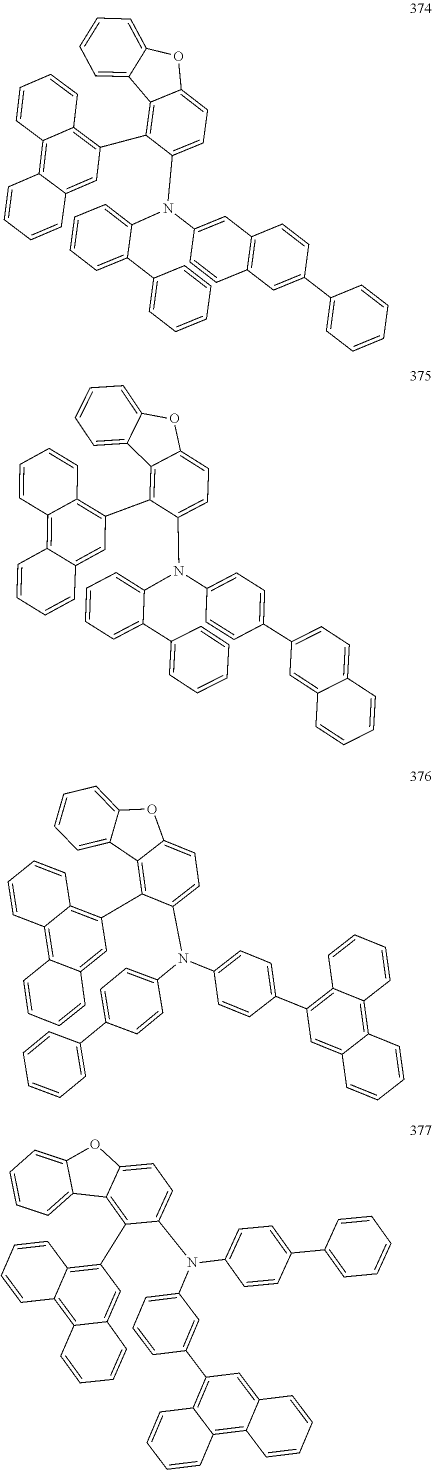 [SMILES: c1ccc(-c2ccc3cc(N(c4ccccc4-c4ccccc4)c4ccc5oc6ccccc6c5c4-c4cc5ccccc5c5ccccc45)ccc3c2)cc1 ; c1ccc(-c2ccccc2N(c2ccc(-c3ccc4ccccc4c3)cc2)c2ccc3oc4ccccc4c3c2-c2cc3ccccc3c3ccccc23)cc1 ; c1ccc(-c2ccc(N(c3ccc(-c4cc5ccccc5c5ccccc45)cc3)c3ccc4oc5ccccc5c4c3-c3cc4ccccc4c4ccccc34)cc2)cc1 ; c1ccc(-c2ccc(N(c3cccc(-c4cc5ccccc5c5ccccc45)c3)c3ccc4oc5ccccc5c4c3-c3cc4ccccc4c4ccccc34)cc2)cc1]

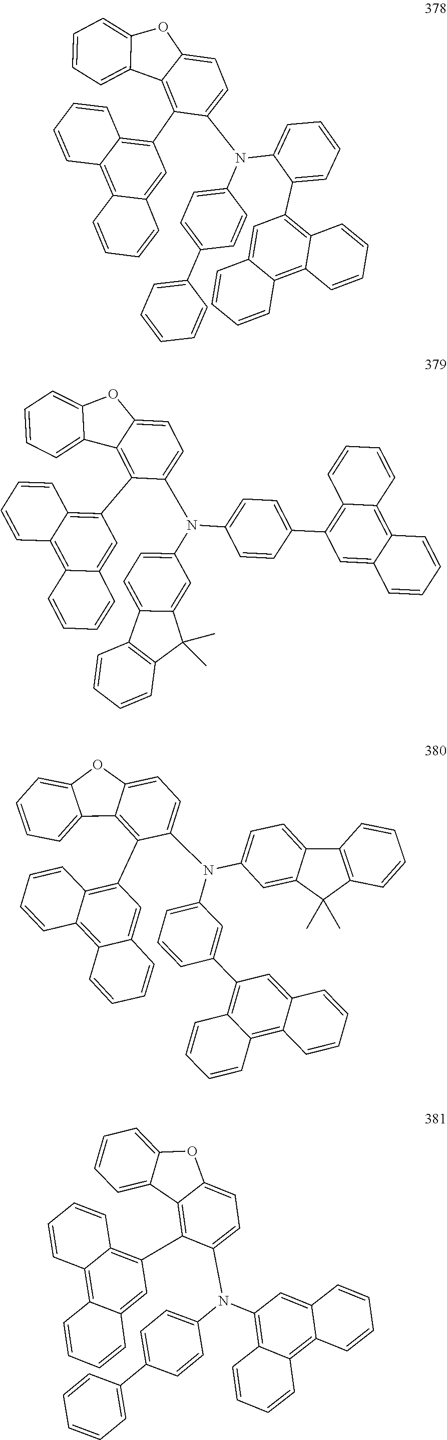 [SMILES: c1ccc(-c2ccc(N(c3ccccc3-c3cc4ccccc4c4ccccc34)c3ccc4oc5ccccc5c4c3-c3cc4ccccc4c4ccccc34)cc2)cc1 ; CC1(C)c2ccccc2-c2ccc(N(c3ccc(-c4cc5ccccc5c5ccccc45)cc3)c3ccc4oc5ccccc5c4c3-c3cc4ccccc4c4ccccc34)cc21 ; CC1(C)c2ccccc2-c2ccc(N(c3cccc(-c4cc5ccccc5c5ccccc45)c3)c3ccc4oc5ccccc5c4c3-c3cc4ccccc4c4ccccc34)cc21 ; c1ccc(-c2ccc(N(c3ccc4oc5ccccc5c4c3-c3cc4ccccc4c4ccccc34)c3cc4ccccc4c4ccccc34)cc2)cc1]

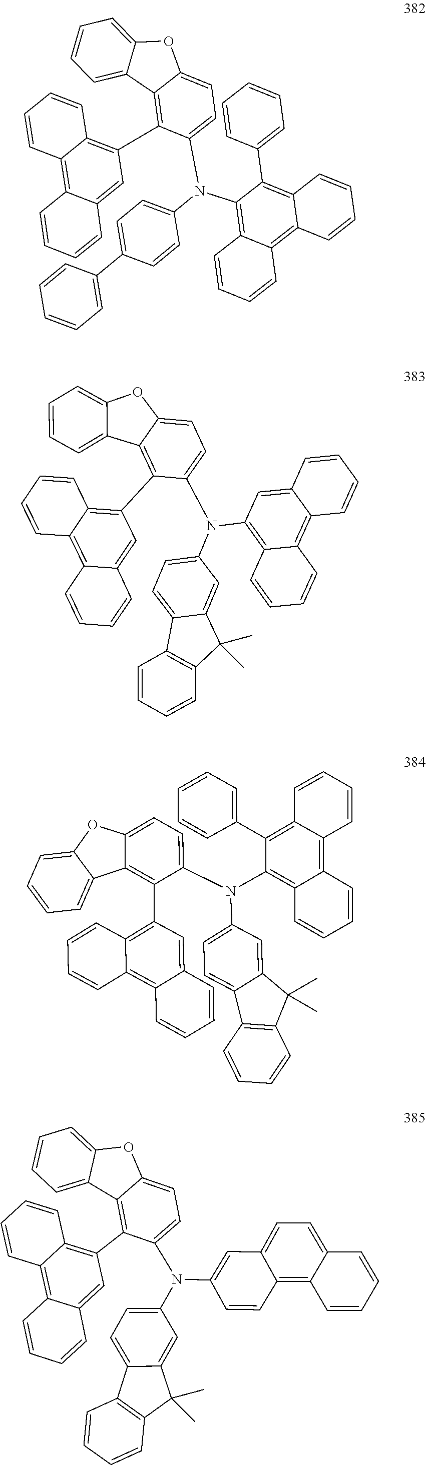 [SMILES: c1ccc(-c2ccc(N(c3ccc4oc5ccccc5c4c3-c3cc4ccccc4c4ccccc34)c3c(-c4ccccc4)c4ccccc4c4ccccc34)cc2)cc1 ; CC1(C)c2ccccc2-c2ccc(N(c3ccc4oc5ccccc5c4c3-c3cc4ccccc4c4ccccc34)c3cc4ccccc4c4ccccc34)cc21 ; CC1(C)c2ccccc2-c2ccc(N(c3ccc4oc5ccccc5c4c3-c3cc4ccccc4c4ccccc34)c3c(-c4ccccc4)c4ccccc4c4ccccc34)cc21 ; CC1(C)c2ccccc2-c2ccc(N(c3ccc4c(ccc5ccccc54)c3)c3ccc4oc5ccccc5c4c3-c3cc4ccccc4c4ccccc34)cc21]

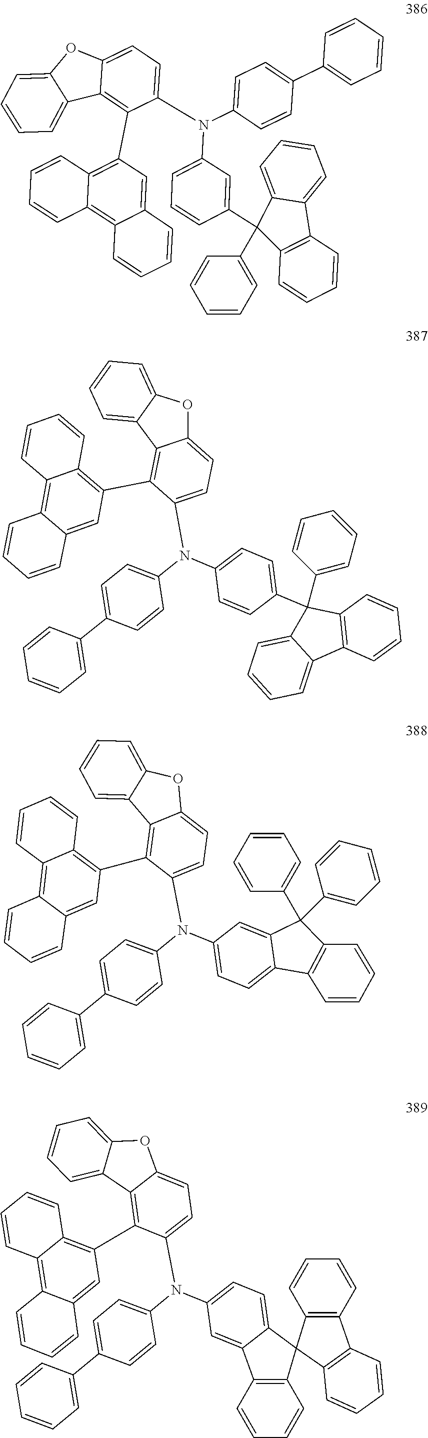 [SMILES: c1ccc(-c2ccc(N(c3cccc(C4(c5ccccc5)c5ccccc5-c5ccccc54)c3)c3ccc4oc5ccccc5c4c3-c3cc4ccccc4c4ccccc34)cc2)cc1 ; c1ccc(-c2ccc(N(c3ccc(C4(c5ccccc5)c5ccccc5-c5ccccc54)cc3)c3ccc4oc5ccccc5c4c3-c3cc4ccccc4c4ccccc34)cc2)cc1 ; c1ccc(-c2ccc(N(c3ccc4c(c3)C(c3ccccc3)(c3ccccc3)c3ccccc3-4)c3ccc4oc5ccccc5c4c3-c3cc4ccccc4c4ccccc34)cc2)cc1 ; c1ccc(-c2ccc(N(c3ccc4c(c3)-c3ccccc3C43c4ccccc4-c4ccccc43)c3ccc4oc5ccccc5c4c3-c3cc4ccccc4c4ccccc34)cc2)cc1]

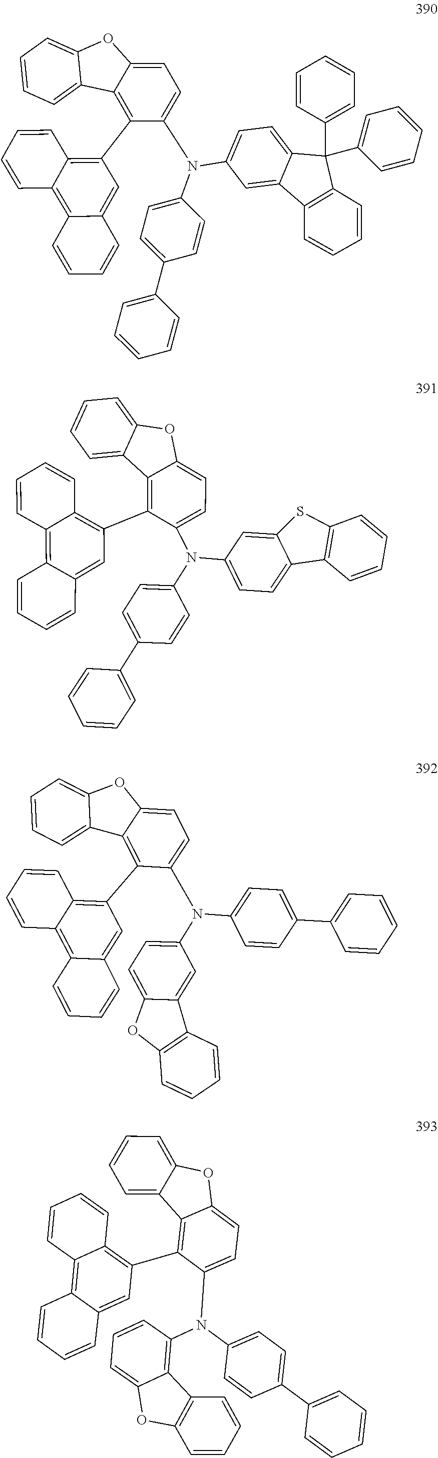

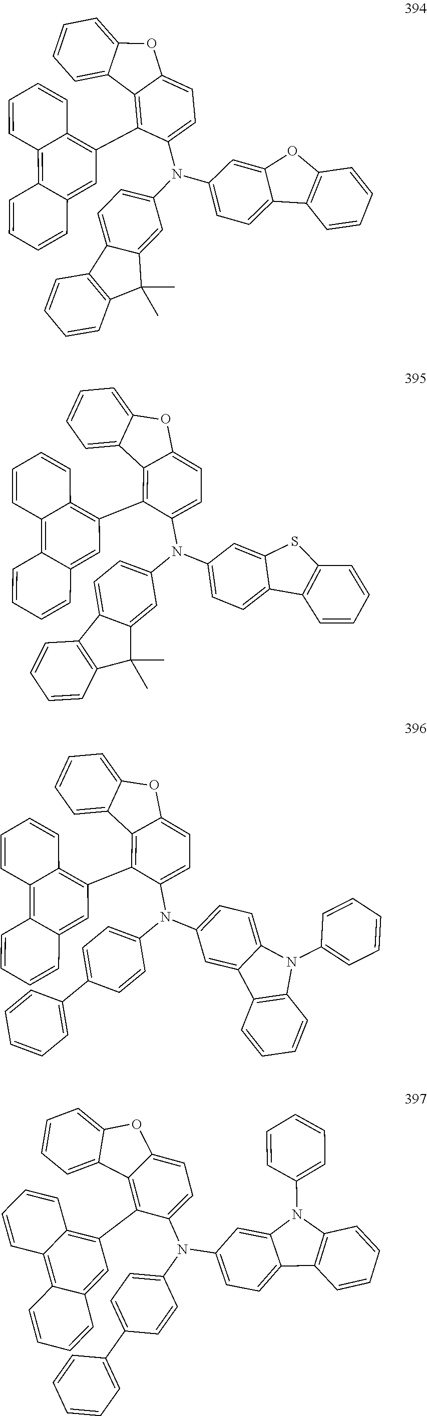 [SMILES: CC1(C)c2ccccc2-c2ccc(N(c3ccc4c(c3)oc3ccccc34)c3ccc4oc5ccccc5c4c3-c3cc4ccccc4c4ccccc34)cc21 ; CC1(C)c2ccccc2-c2ccc(N(c3ccc4c(c3)sc3ccccc34)c3ccc4oc5ccccc5c4c3-c3cc4ccccc4c4ccccc34)cc21 ; c1ccc(-c2ccc(N(c3ccc4c(c3)c3ccccc3n4-c3ccccc3)c3ccc4oc5ccccc5c4c3-c3cc4ccccc4c4ccccc34)cc2)cc1 ; c1ccc(-c2ccc(N(c3ccc4c5ccccc5n(-c5ccccc5)c4c3)c3ccc4oc5ccccc5c4c3-c3cc4ccccc4c4ccccc34)cc2)cc1]

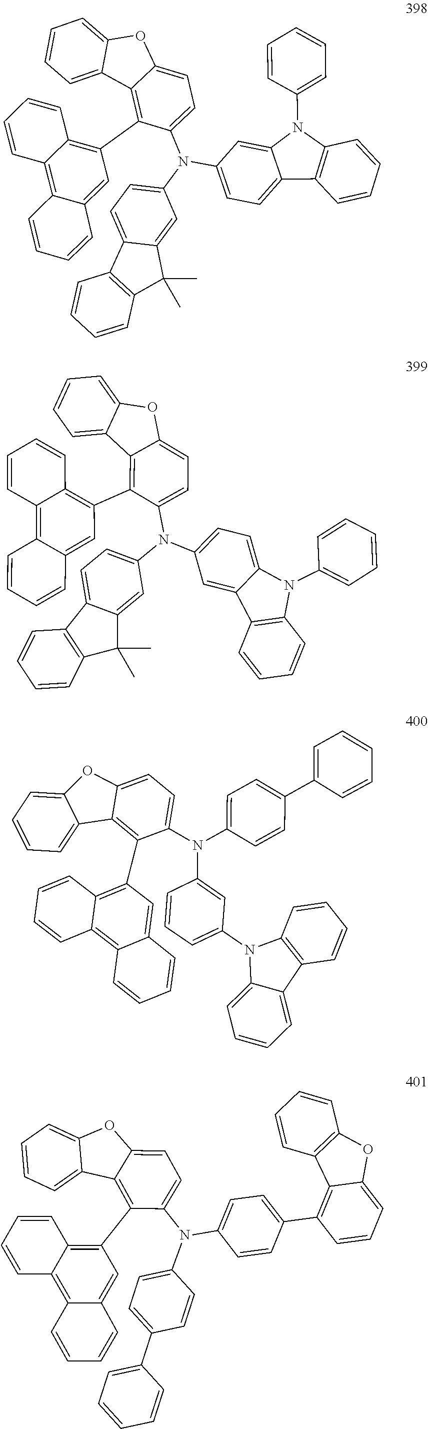

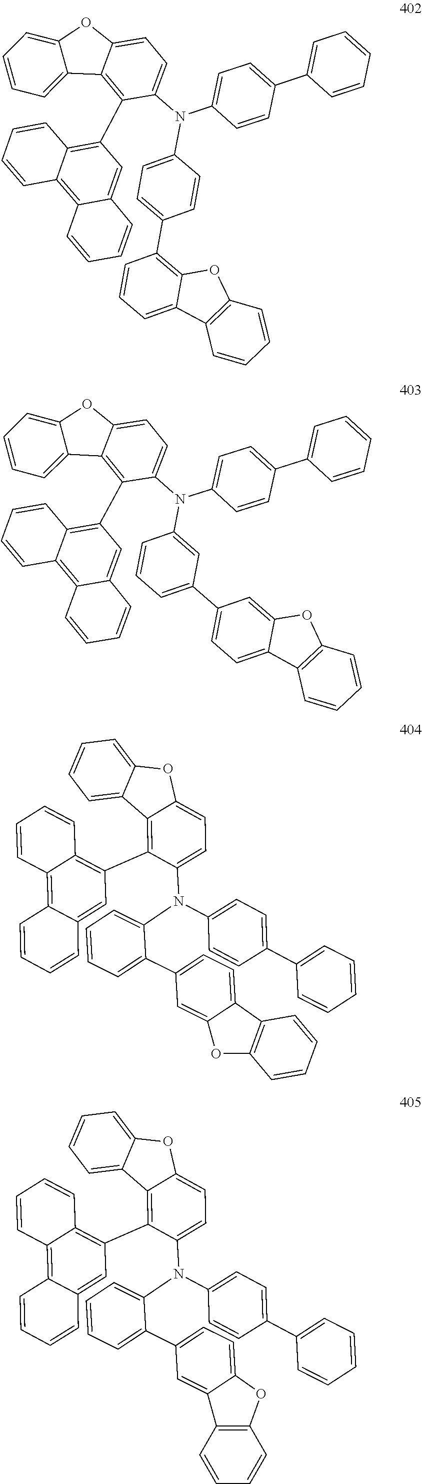 [SMILES: c1ccc(-c2ccc(N(c3ccc(-c4cccc5c4oc4ccccc45)cc3)c3ccc4oc5ccccc5c4c3-c3cc4ccccc4c4ccccc34)cc2)cc1 ; c1ccc(-c2ccc(N(c3cccc(-c4ccc5c(c4)oc4ccccc45)c3)c3ccc4oc5ccccc5c4c3-c3cc4ccccc4c4ccccc34)cc2)cc1 ; c1ccc(-c2ccc(N(c3ccccc3-c3ccc4c(c3)oc3ccccc34)c3ccc4oc5ccccc5c4c3-c3cc4ccccc4c4ccccc34)cc2)cc1 ; c1ccc(-c2ccc(N(c3ccccc3-c3ccc4oc5ccccc5c4c3)c3ccc4oc5ccccc5c4c3-c3cc4ccccc4c4ccccc34)cc2)cc1]

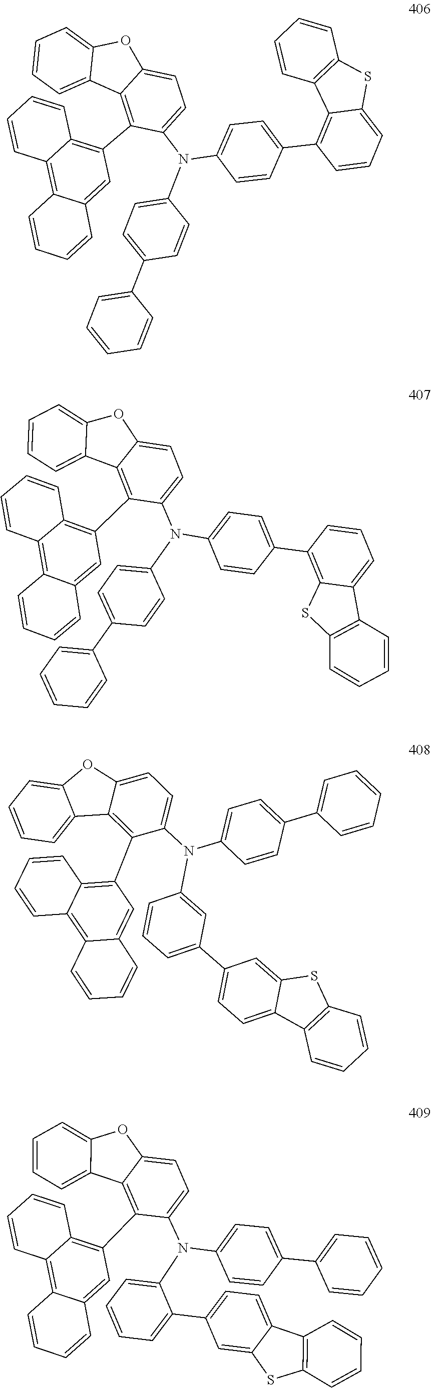 [SMILES: c1ccc(-c2ccc(N(c3ccc(-c4cccc5sc6ccccc6c45)cc3)c3ccc4oc5ccccc5c4c3-c3cc4ccccc4c4ccccc34)cc2)cc1 ; c1ccc(-c2ccc(N(c3ccc(-c4cccc5c4sc4ccccc45)cc3)c3ccc4oc5ccccc5c4c3-c3cc4ccccc4c4ccccc34)cc2)cc1 ; c1ccc(-c2ccc(N(c3cccc(-c4ccc5c(c4)sc4ccccc45)c3)c3ccc4oc5ccccc5c4c3-c3cc4ccccc4c4ccccc34)cc2)cc1 ; c1ccc(-c2ccc(N(c3ccccc3-c3ccc4c(c3)sc3ccccc34)c3ccc4oc5ccccc5c4c3-c3cc4ccccc4c4ccccc34)cc2)cc1]

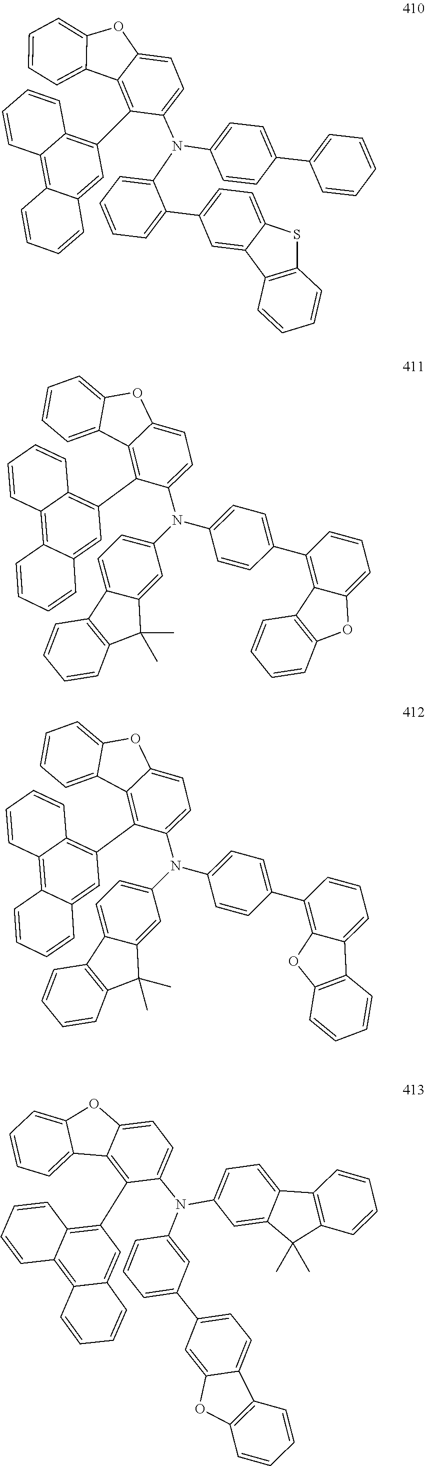

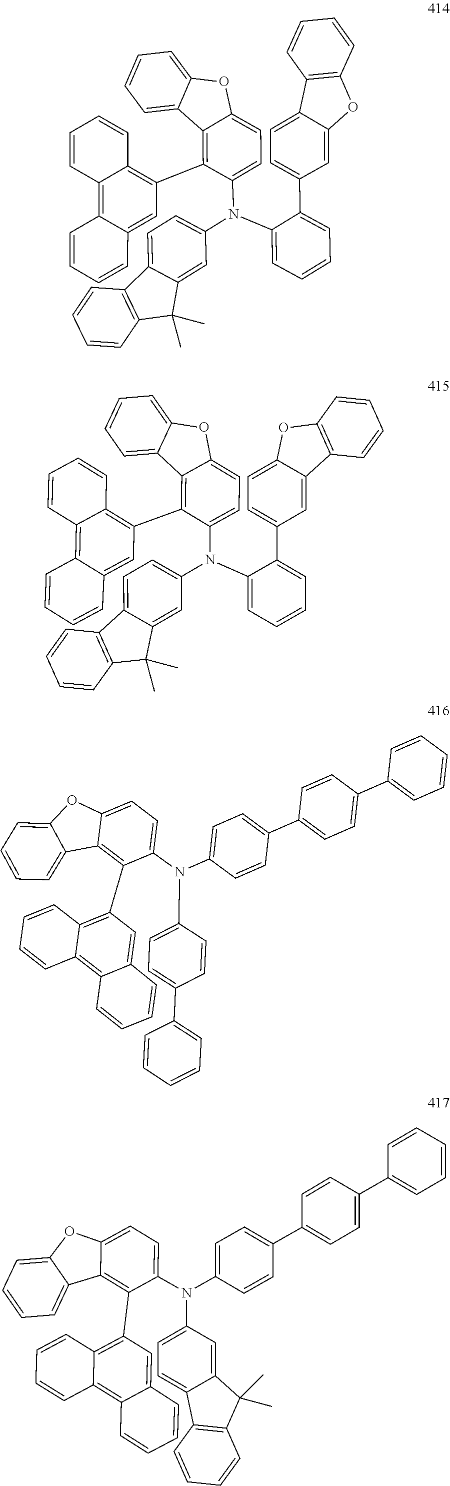 [SMILES: CC1(C)c2ccccc2-c2ccc(N(c3ccccc3-c3ccc4c(c3)oc3ccccc34)c3ccc4oc5ccccc5c4c3-c3cc4ccccc4c4ccccc34)cc21 ; CC1(C)c2ccccc2-c2ccc(N(c3ccccc3-c3ccc4oc5ccccc5c4c3)c3ccc4oc5ccccc5c4c3-c3cc4ccccc4c4ccccc34)cc21 ; c1ccc(-c2ccc(-c3ccc(N(c4ccc(-c5ccccc5)cc4)c4ccc5oc6ccccc6c5c4-c4cc5ccccc5c5ccccc45)cc3)cc2)cc1 ; CC1(C)c2ccccc2-c2ccc(N(c3ccc(-c4ccc(-c5ccccc5)cc4)cc3)c3ccc4oc5ccccc5c4c3-c3cc4ccccc4c4ccccc34)cc21]

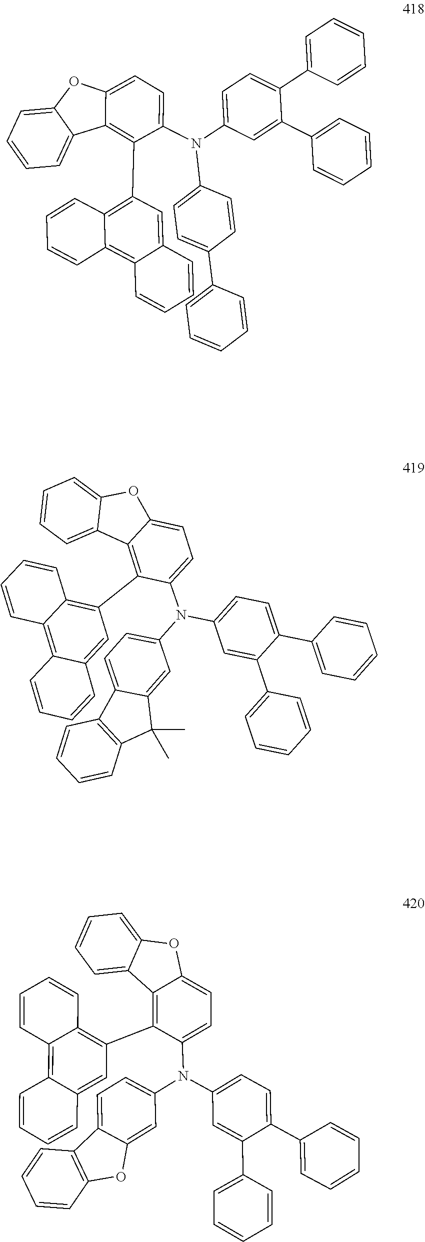 [SMILES: c1ccc(-c2ccc(N(c3ccc(-c4ccccc4)c(-c4ccccc4)c3)c3ccc4oc5ccccc5c4c3-c3cc4ccccc4c4ccccc34)cc2)cc1 ; CC1(C)c2ccccc2-c2ccc(N(c3ccc(-c4ccccc4)c(-c4ccccc4)c3)c3ccc4oc5ccccc5c4c3-c3cc4ccccc4c4ccccc34)cc21 ; c1ccc(-c2ccc(N(c3ccc4c(c3)oc3ccccc34)c3ccc4oc5ccccc5c4c3-c3cc4ccccc4c4ccccc34)cc2-c2ccccc2)cc1]

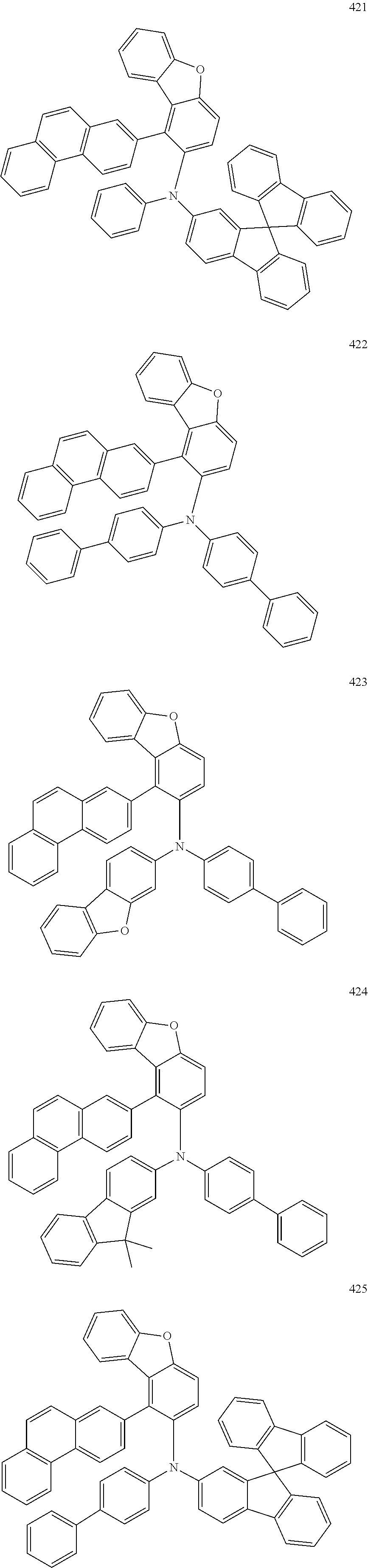 [SMILES: c1ccc(N(c2ccc3c(c2)C2(c4ccccc4-c4ccccc42)c2ccccc2-3)c2ccc3oc4ccccc4c3c2-c2ccc3c(ccc4ccccc43)c2)cc1 ; c1ccc(-c2ccc(N(c3ccc(-c4ccccc4)cc3)c3ccc4oc5ccccc5c4c3-c3ccc4c(ccc5ccccc54)c3)cc2)cc1 ; c1ccc(-c2ccc(N(c3ccc4c(c3)oc3ccccc34)c3ccc4oc5ccccc5c4c3-c3ccc4c(ccc5ccccc54)c3)cc2)cc1 ; CC1(C)c2ccccc2-c2ccc(N(c3ccc(-c4ccccc4)cc3)c3ccc4oc5ccccc5c4c3-c3ccc4c(ccc5ccccc54)c3)cc21 ; c1ccc(-c2ccc(N(c3ccc4c(c3)C3(c5ccccc5-c5ccccc53)c3ccccc3-4)c3ccc4oc5ccccc5c4c3-c3ccc4c(ccc5ccccc54)c3)cc2)cc1]

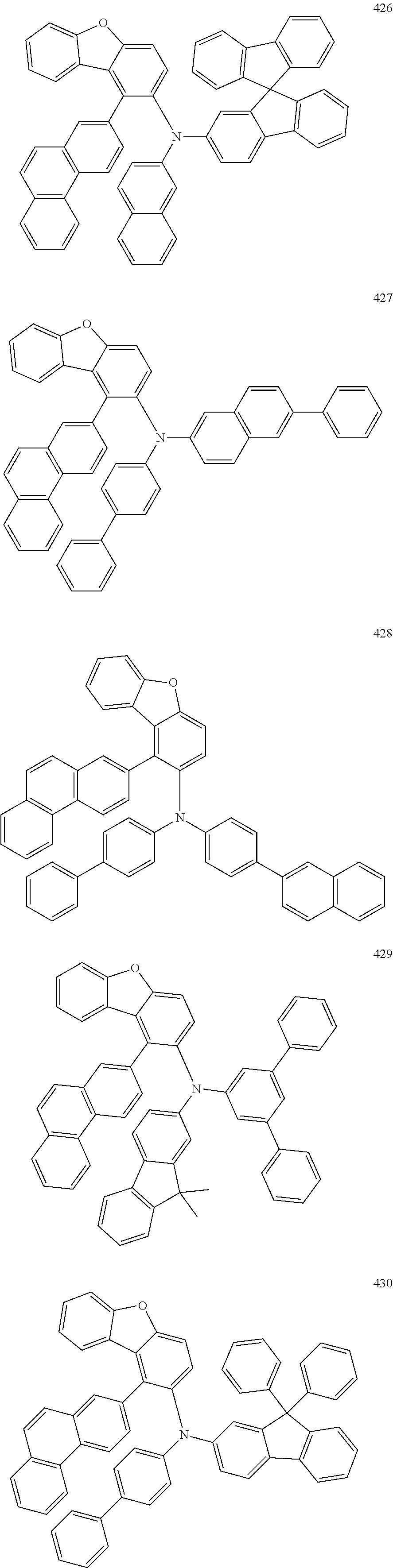

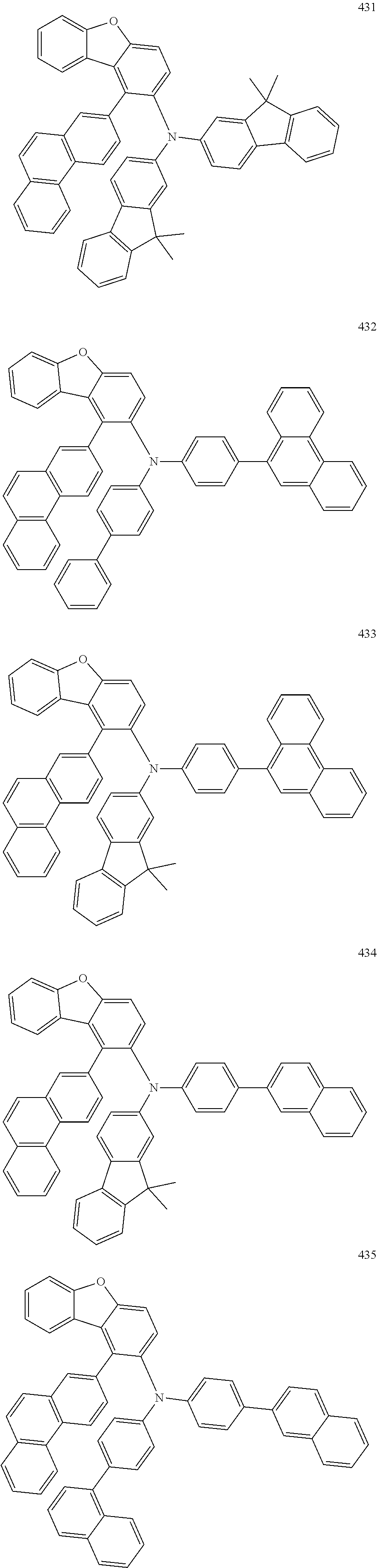

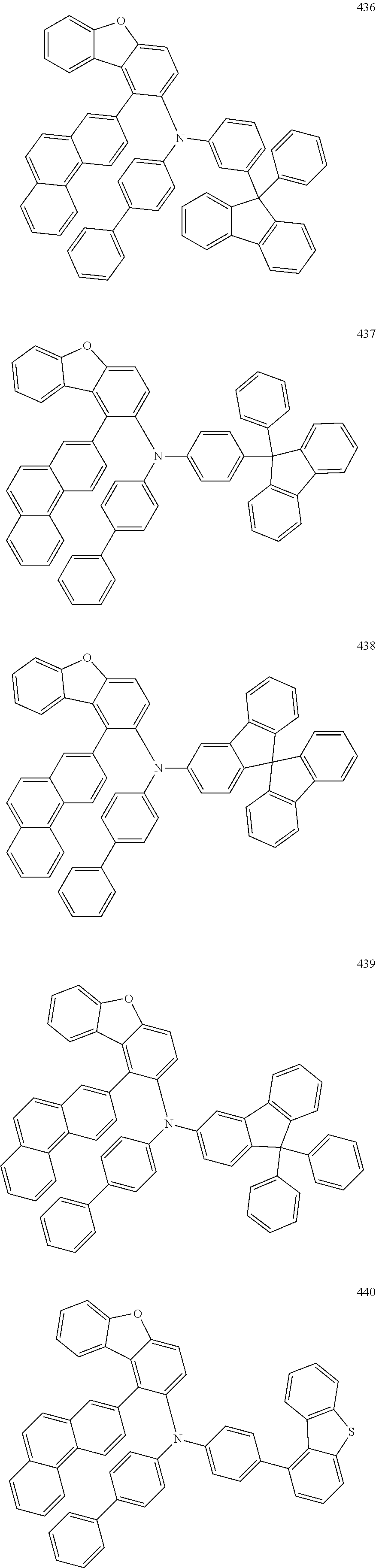

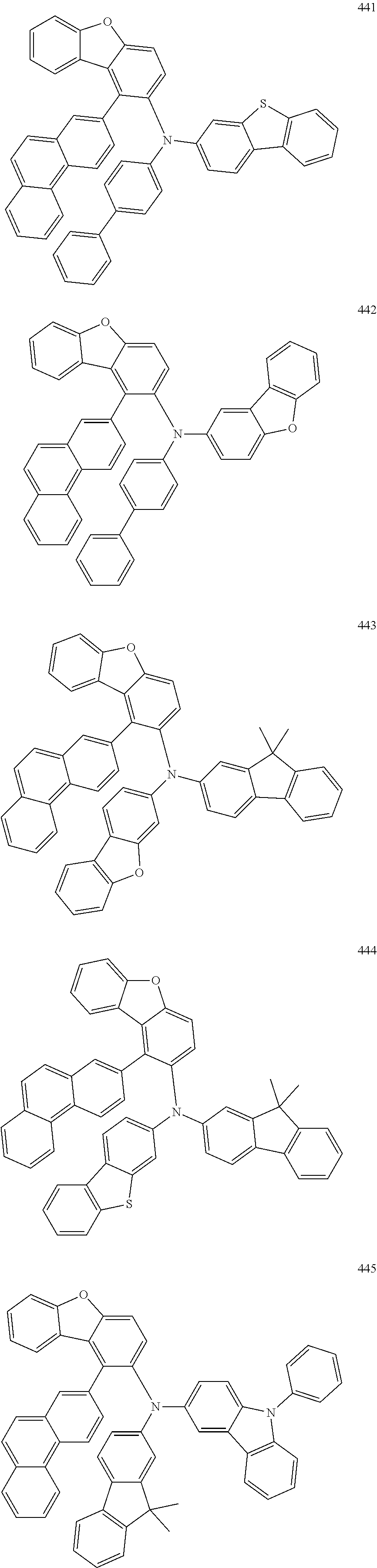

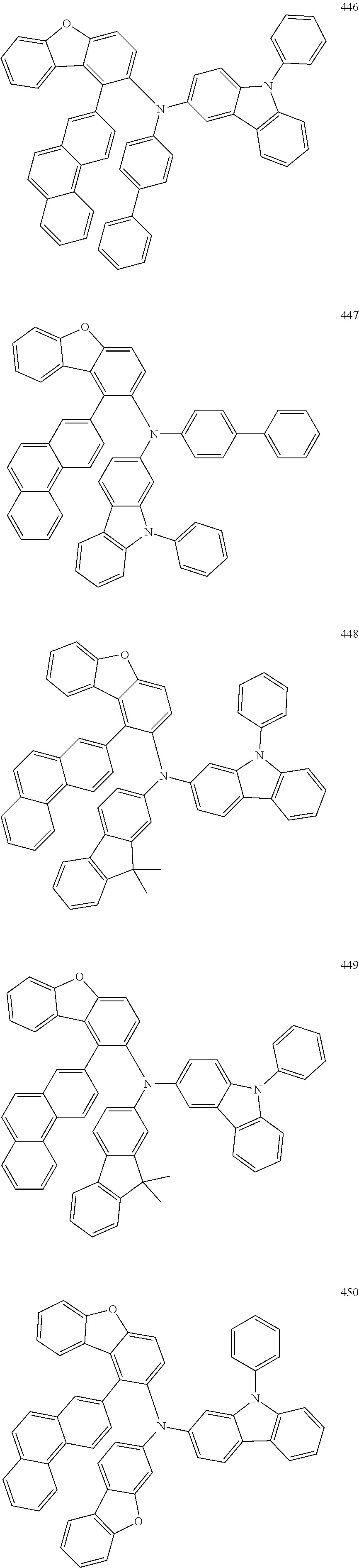

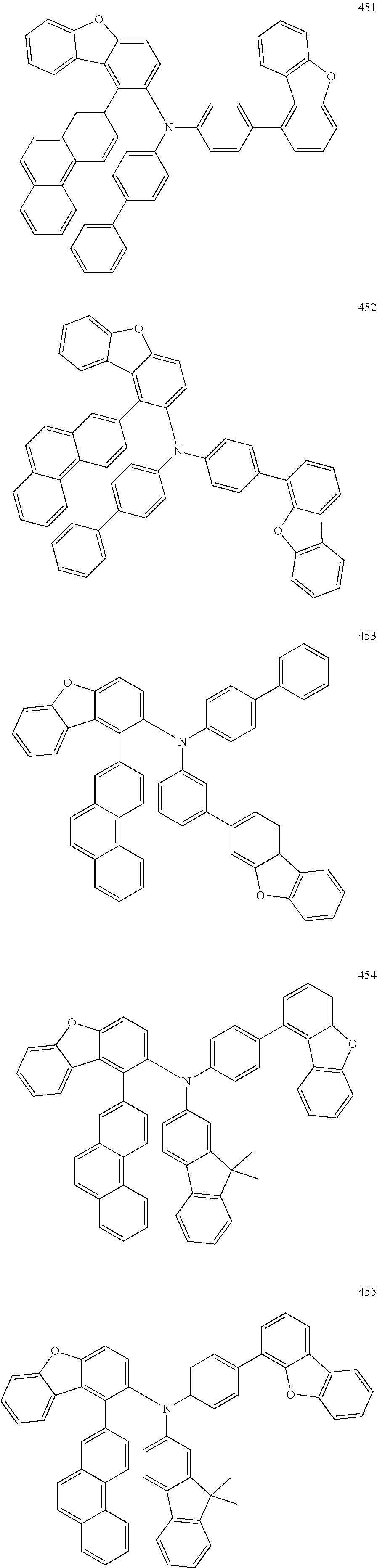

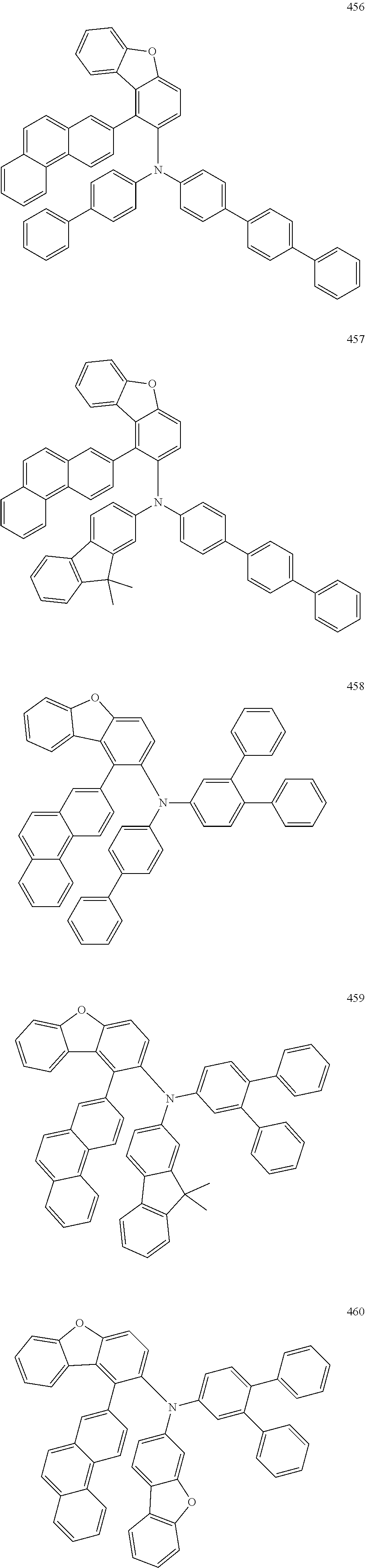

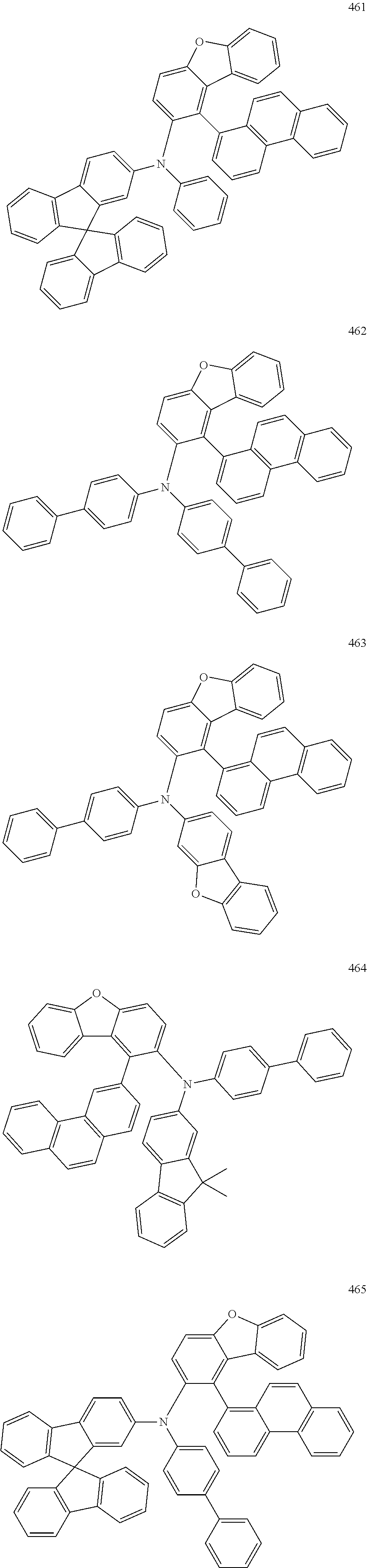 [SMILES: c1ccc(N(c2ccc3c(c2)C2(c4ccccc4-c4ccccc42)c2ccccc2-3)c2ccc3oc4ccccc4c3c2-c2cccc3c2ccc2ccccc23)cc1 ; c1ccc(-c2ccc(N(c3ccc(-c4ccccc4)cc3)c3ccc4oc5ccccc5c4c3-c3cccc4c3ccc3ccccc34)cc2)cc1 ; c1ccc(-c2ccc(N(c3ccc4c(c3)oc3ccccc34)c3ccc4oc5ccccc5c4c3-c3cccc4c3ccc3ccccc34)cc2)cc1 ; CC1(C)c2ccccc2-c2ccc(N(c3ccc(-c4ccccc4)cc3)c3ccc4oc5ccccc5c4c3-c3ccc4ccc5ccccc5c4c3)cc21 ; c1ccc(-c2ccc(N(c3ccc4c(c3)C3(c5ccccc5-c5ccccc53)c3ccccc3-4)c3ccc4oc5ccccc5c4c3-c3cccc4c3ccc3ccccc34)cc2)cc1]

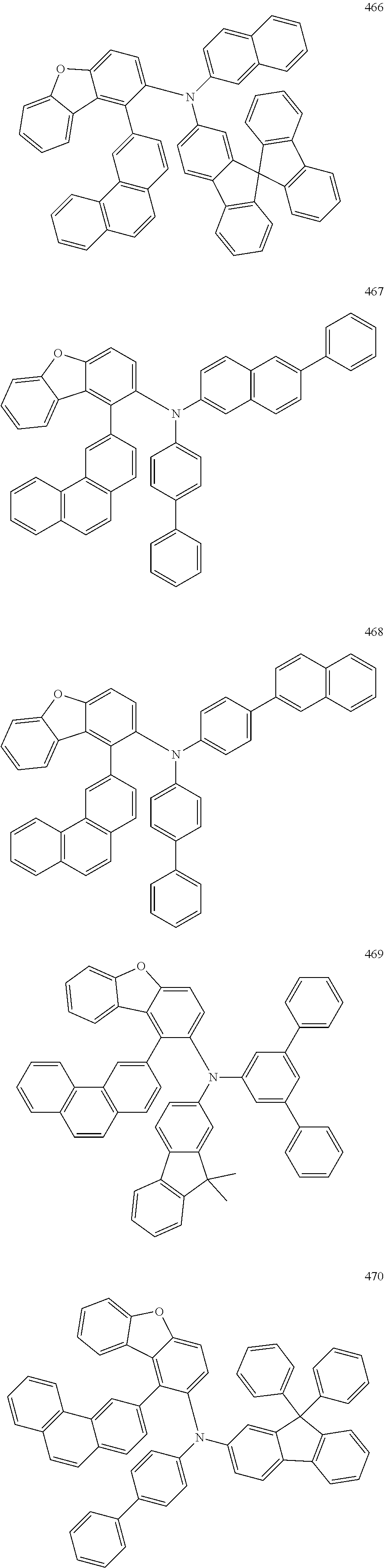 [SMILES: c1ccc2c(c1)-c1ccccc1C21c2ccccc2-c2ccc(N(c3ccc4ccccc4c3)c3ccc4oc5ccccc5c4c3-c3ccc4ccc5ccccc5c4c3)cc21 ; c1ccc(-c2ccc(N(c3ccc4cc(-c5ccccc5)ccc4c3)c3ccc4oc5ccccc5c4c3-c3ccc4ccc5ccccc5c4c3)cc2)cc1 ; c1ccc(-c2ccc(N(c3ccc(-c4ccc5ccccc5c4)cc3)c3ccc4oc5ccccc5c4c3-c3ccc4ccc5ccccc5c4c3)cc2)cc1 ; CC1(C)c2ccccc2-c2ccc(N(c3cc(-c4ccccc4)cc(-c4ccccc4)c3)c3ccc4oc5ccccc5c4c3-c3ccc4ccc5ccccc5c4c3)cc21 ; c1ccc(-c2ccc(N(c3ccc4c(c3)C(c3ccccc3)(c3ccccc3)c3ccccc3-4)c3ccc4oc5ccccc5c4c3-c3ccc4ccc5ccccc5c4c3)cc2)cc1]

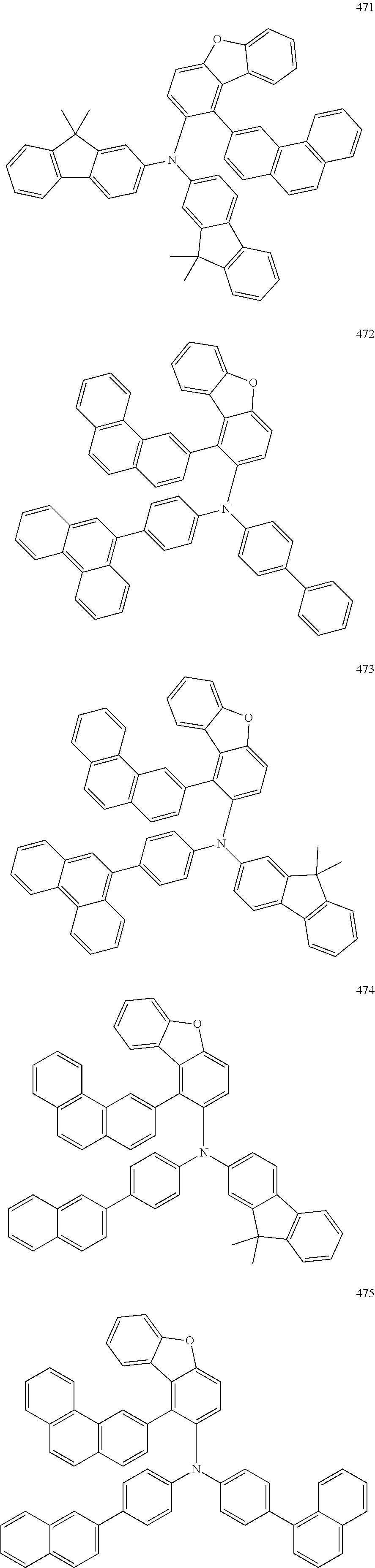 [SMILES: CC1(C)c2ccccc2-c2ccc(N(c3ccc4c(c3)C(C)(C)c3ccccc3-4)c3ccc4oc5ccccc5c4c3-c3ccc4ccc5ccccc5c4c3)cc21 ; c1ccc(-c2ccc(N(c3ccc(-c4cc5ccccc5c5ccccc45)cc3)c3ccc4oc5ccccc5c4c3-c3ccc4ccc5ccccc5c4c3)cc2)cc1 ; CC1(C)c2ccccc2-c2ccc(N(c3ccc(-c4cc5ccccc5c5ccccc45)cc3)c3ccc4oc5ccccc5c4c3-c3ccc4ccc5ccccc5c4c3)cc21 ; CC1(C)c2ccccc2-c2ccc(N(c3ccc(-c4ccc5ccccc5c4)cc3)c3ccc4oc5ccccc5c4c3-c3ccc4ccc5ccccc5c4c3)cc21 ; c1ccc2cc(-c3ccc(N(c4ccc(-c5cccc6ccccc56)cc4)c4ccc5oc6ccccc6c5c4-c4ccc5ccc6ccccc6c5c4)cc3)ccc2c1]

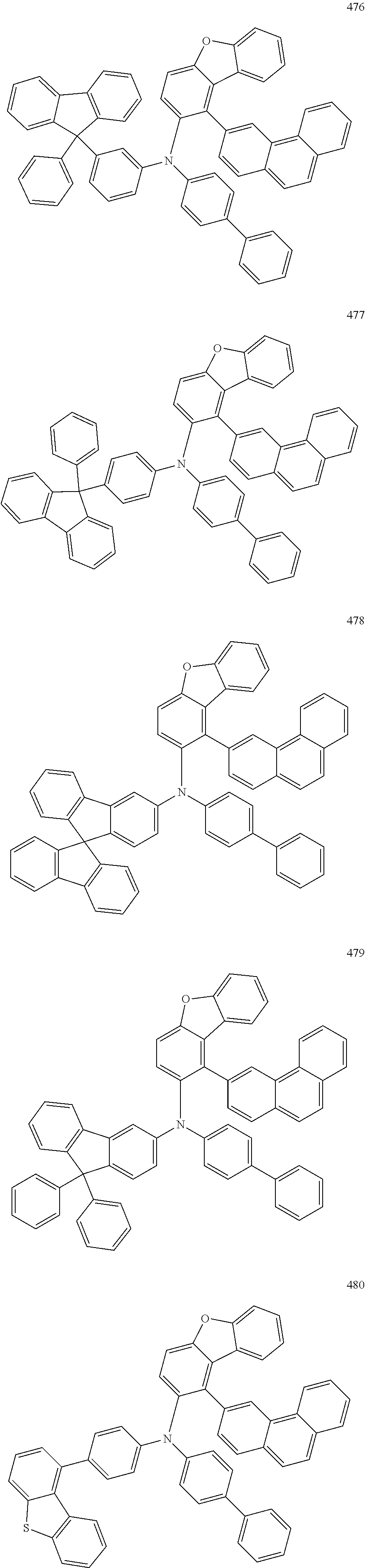

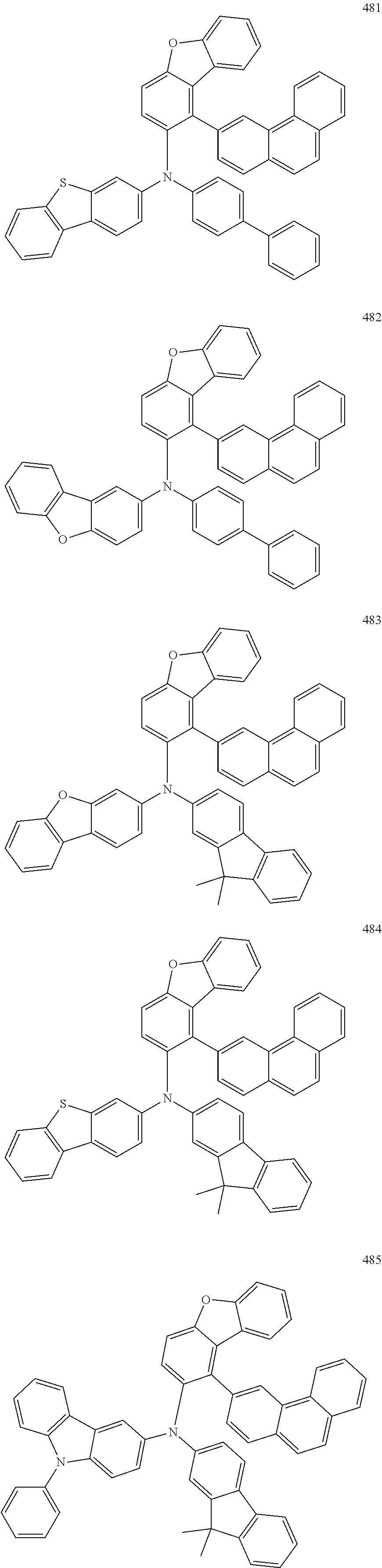

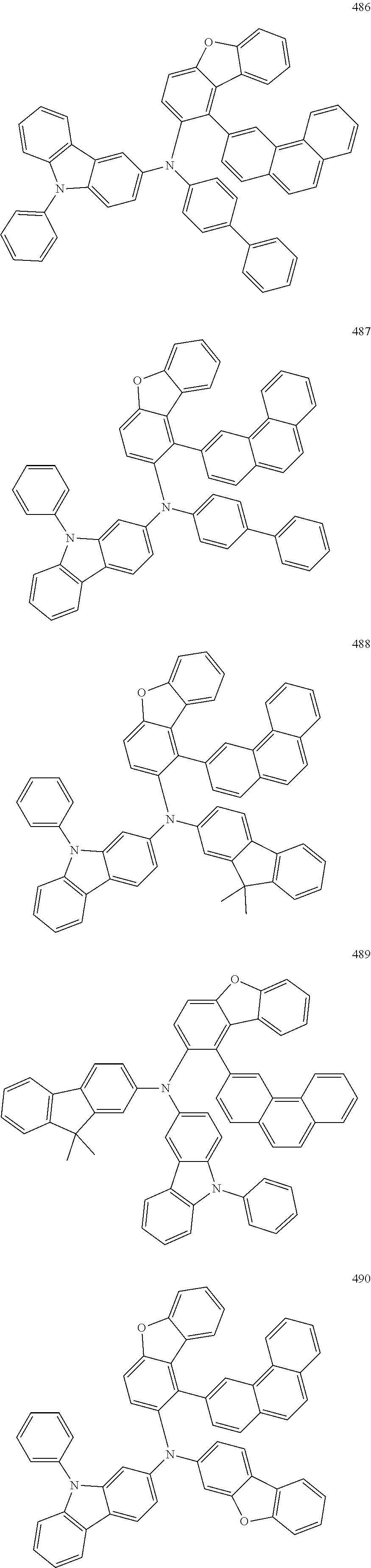 [SMILES: c1ccc(-c2ccc(N(c3ccc4c(c3)c3ccccc3n4-c3ccccc3)c3ccc4oc5ccccc5c4c3-c3ccc4ccc5ccccc5c4c3)cc2)cc1 ; c1ccc(-c2ccc(N(c3ccc4c5ccccc5n(-c5ccccc5)c4c3)c3ccc4oc5ccccc5c4c3-c3ccc4ccc5ccccc5c4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4ccc5ccccc5c4c3)cc21 ; CC1(C)c2ccccc2-c2ccc(N(c3ccc4c(c3)c3ccccc3n4-c3ccccc3)c3ccc4oc5ccccc5c4c3-c3ccc4ccc5ccccc5c4c3)cc21 ; c1ccc(-n2c3ccccc3c3ccc(N(c4ccc5c(c4)oc4ccccc45)c4ccc5oc6ccccc6c5c4-c4ccc5ccc6ccccc6c5c4)cc32)cc1]

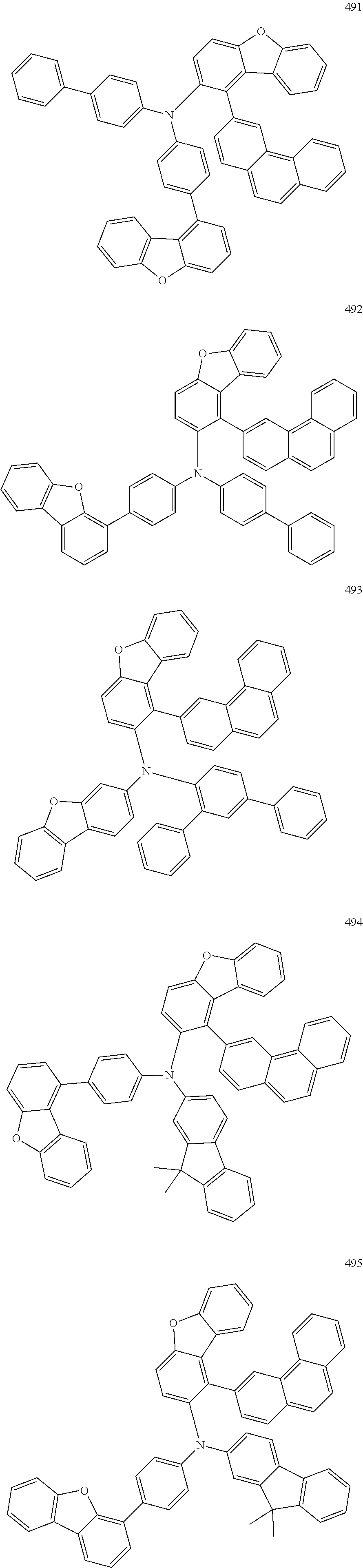 [SMILES: c1ccc(-c2ccc(N(c3ccc(-c4cccc5oc6ccccc6c45)cc3)c3ccc4oc5ccccc5c4c3-c3ccc4ccc5ccccc5c4c3)cc2)cc1 ; c1ccc(-c2ccc(N(c3ccc(-c4cccc5c4oc4ccccc45)cc3)c3ccc4oc5ccccc5c4c3-c3ccc4ccc5ccccc5c4c3)cc2)cc1 ; c1ccc(-c2ccc(N(c3ccc4c(c3)oc3ccccc34)c3ccc4oc5ccccc5c4c3-c3ccc4ccc5ccccc5c4c3)c(-c3ccccc3)c2)cc1 ; CC1(C)c2ccccc2-c2ccc(N(c3ccc(-c4cccc5oc6ccccc6c45)cc3)c3ccc4oc5ccccc5c4c3-c3ccc4ccc5ccccc5c4c3)cc21 ; CC1(C)c2ccccc2-c2ccc(N(c3ccc(-c4cccc5c4oc4ccccc45)cc3)c3ccc4oc5ccccc5c4c3-c3ccc4ccc5ccccc5c4c3)cc21]

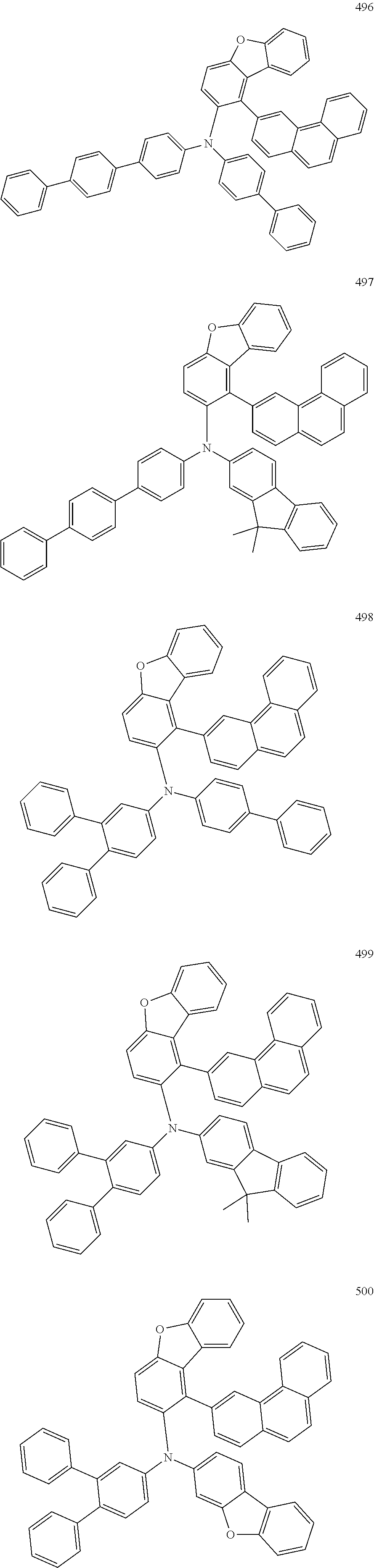 [SMILES: c1ccc(-c2ccc(-c3ccc(N(c4ccc(-c5ccccc5)cc4)c4ccc5oc6ccccc6c5c4-c4ccc5ccc6ccccc6c5c4)cc3)cc2)cc1 ; CC1(C)c2ccccc2-c2ccc(N(c3ccc(-c4ccc(-c5ccccc5)cc4)cc3)c3ccc4oc5ccccc5c4c3-c3ccc4ccc5ccccc5c4c3)cc21 ; c1ccc(-c2ccc(N(c3ccc(-c4ccccc4)c(-c4ccccc4)c3)c3ccc4oc5ccccc5c4c3-c3ccc4ccc5ccccc5c4c3)cc2)cc1 ; CC1(C)c2ccccc2-c2ccc(N(c3ccc(-c4ccccc4)c(-c4ccccc4)c3)c3ccc4oc5ccccc5c4c3-c3ccc4ccc5ccccc5c4c3)cc21 ; c1ccc(-c2ccc(N(c3ccc4c(c3)oc3ccccc34)c3ccc4oc5ccccc5c4c3-c3ccc4ccc5ccccc5c4c3)cc2-c2ccccc2)cc1]

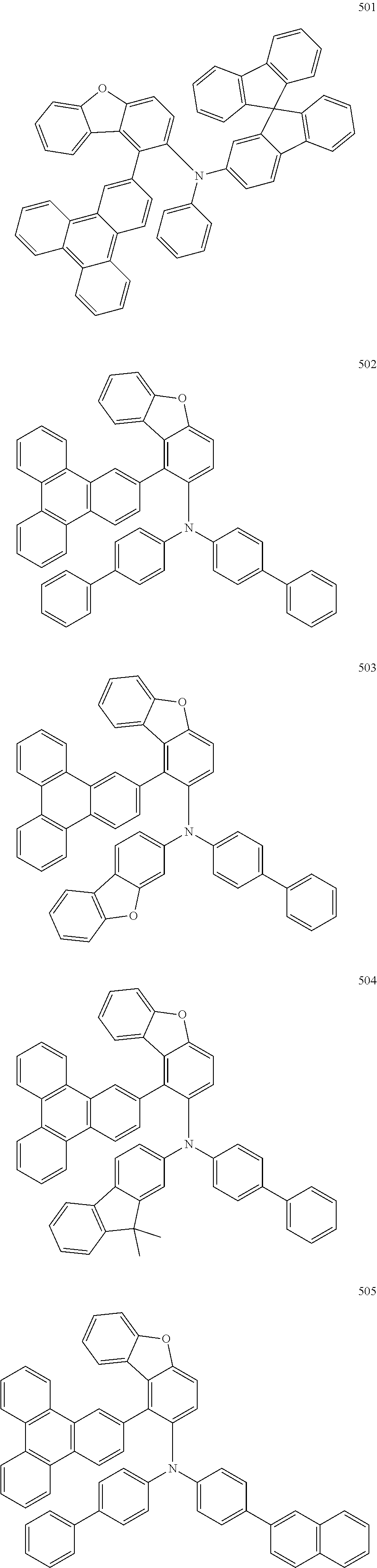 [SMILES: c1ccc(N(c2ccc3c(c2)C2(c4ccccc4-c4ccccc42)c2ccccc2-3)c2ccc3oc4ccccc4c3c2-c2ccc3c4ccccc4c4ccccc4c3c2)cc1 ; c1ccc(-c2ccc(N(c3ccc(-c4ccccc4)cc3)c3ccc4oc5ccccc5c4c3-c3ccc4c5ccccc5c5ccccc5c4c3)cc2)cc1 ; c1ccc(-c2ccc(N(c3ccc4c(c3)oc3ccccc34)c3ccc4oc5ccccc5c4c3-c3ccc4c5ccccc5c5ccccc5c4c3)cc2)cc1 ; CC1(C)c2ccccc2-c2ccc(N(c3ccc(-c4ccccc4)cc3)c3ccc4oc5ccccc5c4c3-c3ccc4c5ccccc5c5ccccc5c4c3)cc21 ; c1ccc(-c2ccc(N(c3ccc(-c4ccc5ccccc5c4)cc3)c3ccc4oc5ccccc5c4c3-c3ccc4c5ccccc5c5ccccc5c4c3)cc2)cc1]

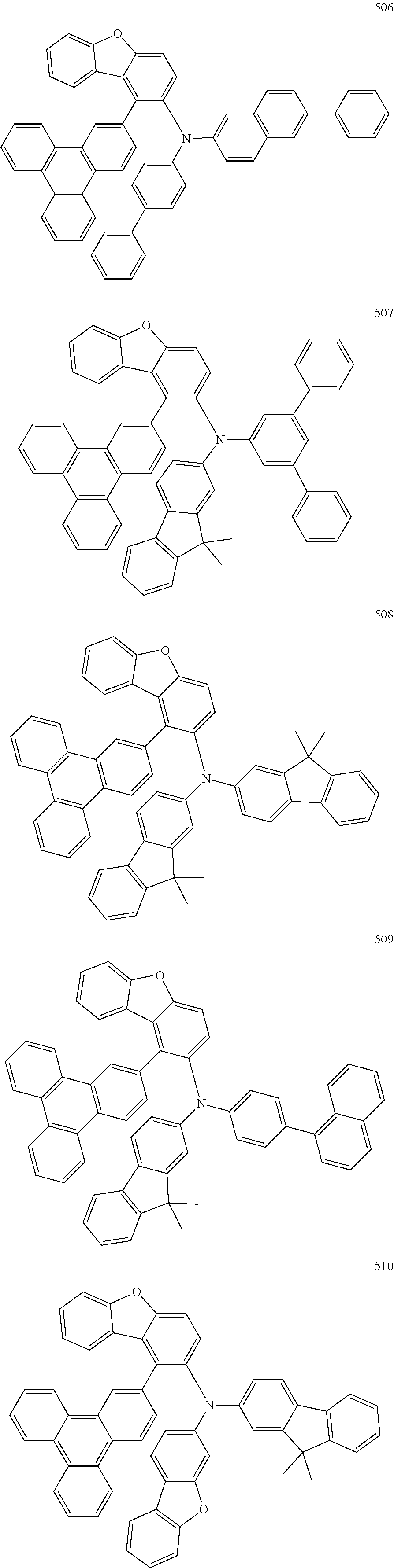

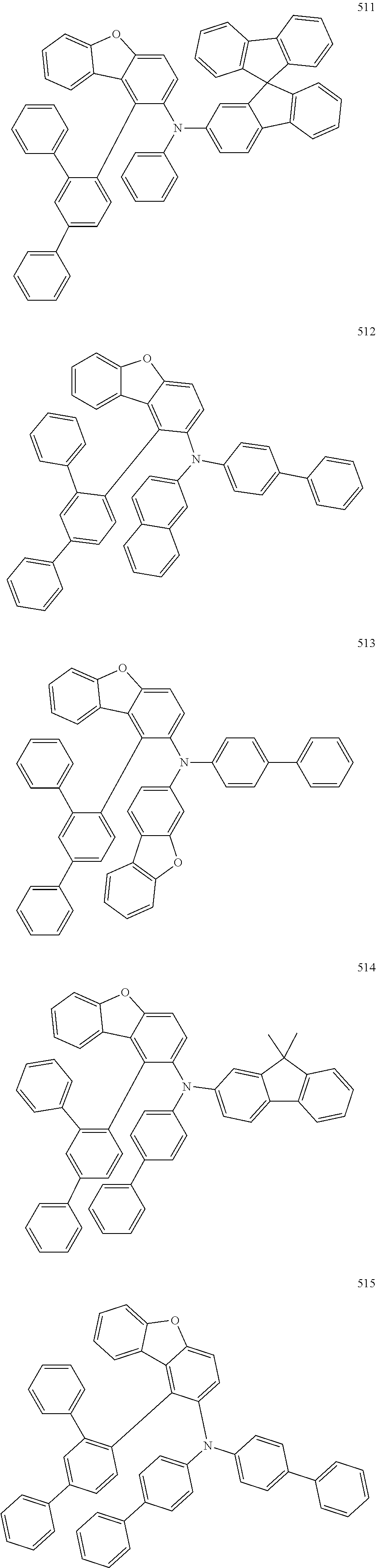 [SMILES: c1ccc(-c2ccc(-c3c(N(c4ccccc4)c4ccc5c(c4)C4(c6ccccc6-c6ccccc64)c4ccccc4-5)ccc4oc5ccccc5c34)c(-c3ccccc3)c2)cc1 ; c1ccc(-c2ccc(N(c3ccc4ccccc4c3)c3ccc4oc5ccccc5c4c3-c3ccc(-c4ccccc4)cc3-c3ccccc3)cc2)cc1 ; c1ccc(-c2ccc(N(c3ccc4c(c3)oc3ccccc34)c3ccc4oc5ccccc5c4c3-c3ccc(-c4ccccc4)cc3-c3ccccc3)cc2)cc1 ; CC1(C)c2ccccc2-c2ccc(N(c3ccc(-c4ccccc4)cc3)c3ccc4oc5ccccc5c4c3-c3ccc(-c4ccccc4)cc3-c3ccccc3)cc21 ; c1ccc(-c2ccc(N(c3ccc(-c4ccccc4)cc3)c3ccc4oc5ccccc5c4c3-c3ccc(-c4ccccc4)cc3-c3ccccc3)cc2)cc1]

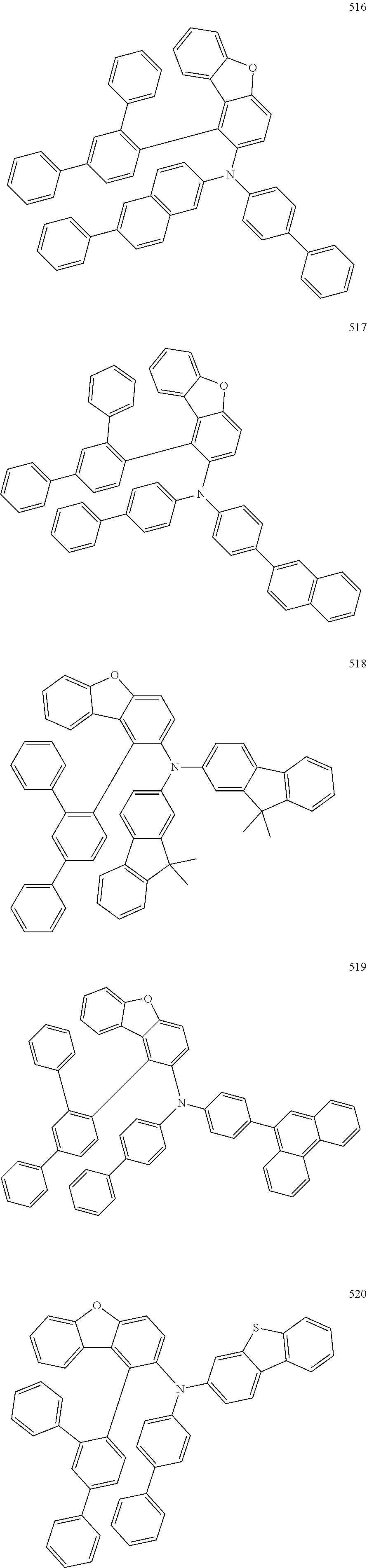

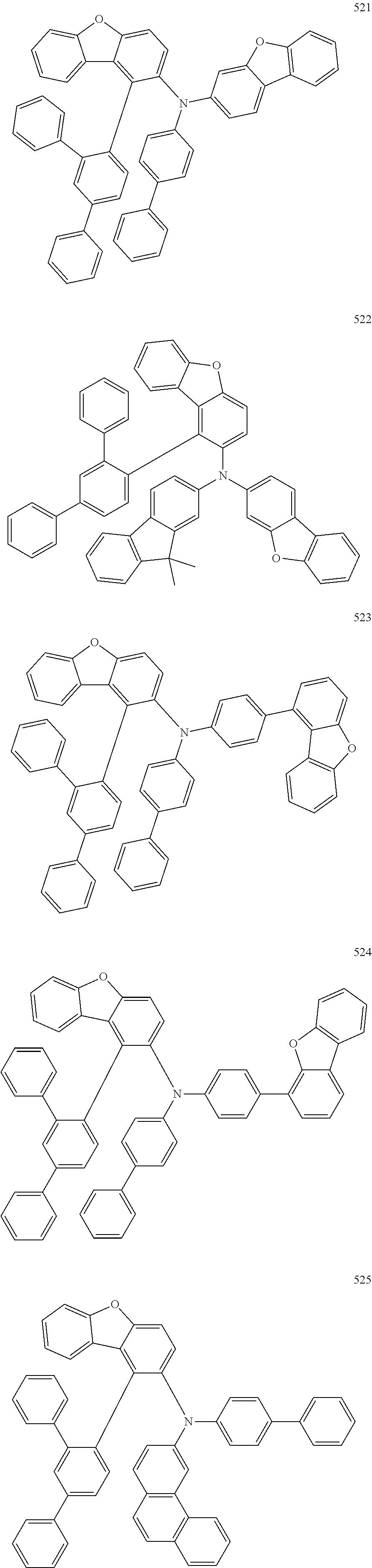 [SMILES: c1ccc(-c2ccc(N(c3ccc4c(c3)oc3ccccc34)c3ccc4oc5ccccc5c4c3-c3ccc(-c4ccccc4)cc3-c3ccccc3)cc2)cc1 ; CC1(C)c2ccccc2-c2ccc(N(c3ccc4c(c3)oc3ccccc34)c3ccc4oc5ccccc5c4c3-c3ccc(-c4ccccc4)cc3-c3ccccc3)cc21 ; c1ccc(-c2ccc(N(c3ccc(-c4cccc5oc6ccccc6c45)cc3)c3ccc4oc5ccccc5c4c3-c3ccc(-c4ccccc4)cc3-c3ccccc3)cc2)cc1 ; c1ccc(-c2ccc(N(c3ccc(-c4cccc5c4oc4ccccc45)cc3)c3ccc4oc5ccccc5c4c3-c3ccc(-c4ccccc4)cc3-c3ccccc3)cc2)cc1 ; c1ccc(-c2ccc(N(c3ccc4ccc5ccccc5c4c3)c3ccc4oc5ccccc5c4c3-c3ccc(-c4ccccc4)cc3-c3ccccc3)cc2)cc1]

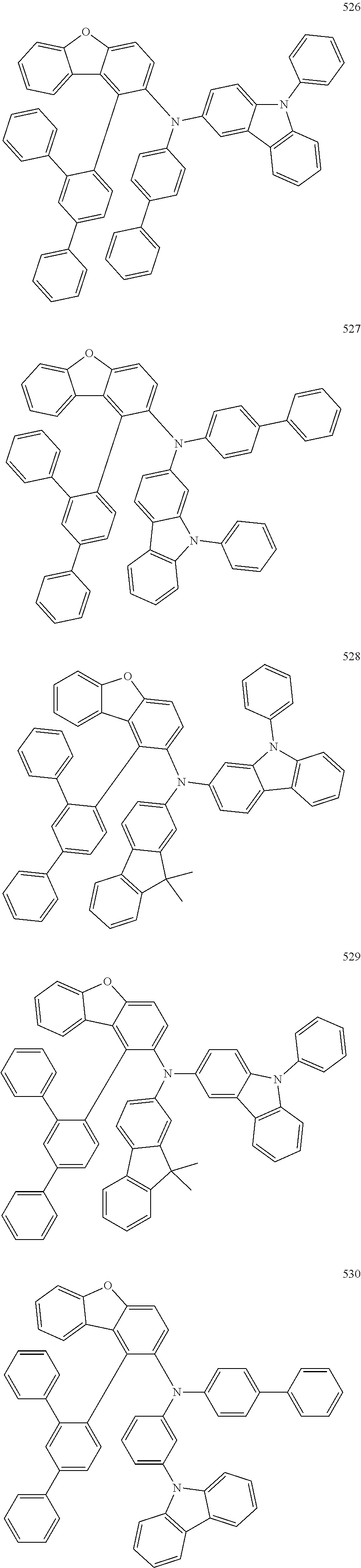

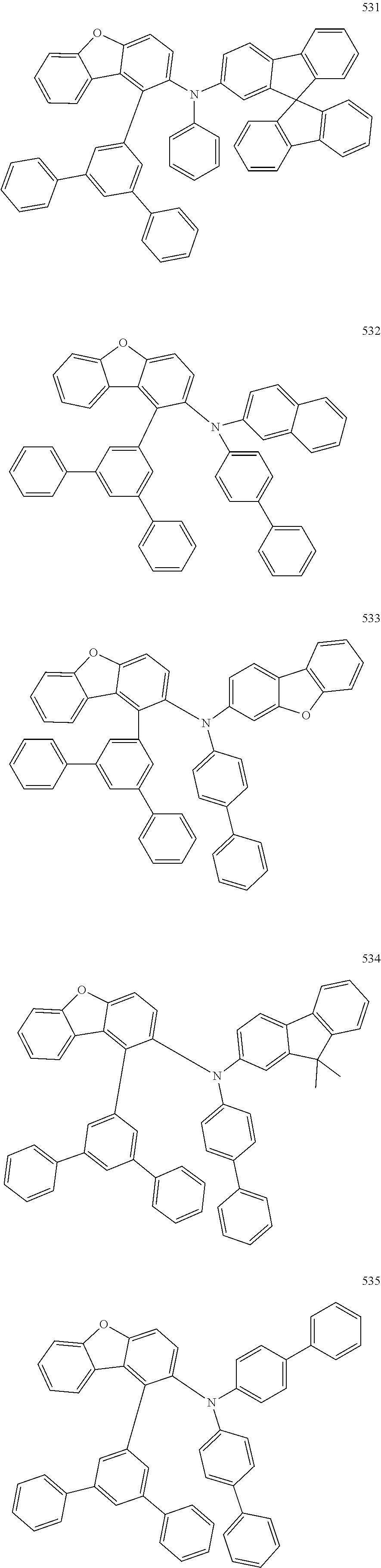 [SMILES: c1ccc(-c2cc(-c3ccccc3)cc(-c3c(N(c4ccccc4)c4ccc5c(c4)C4(c6ccccc6-c6ccccc64)c4ccccc4-5)ccc4oc5ccccc5c34)c2)cc1 ; c1ccc(-c2ccc(N(c3ccc4ccccc4c3)c3ccc4oc5ccccc5c4c3-c3cc(-c4ccccc4)cc(-c4ccccc4)c3)cc2)cc1 ; c1ccc(-c2ccc(N(c3ccc4c(c3)oc3ccccc34)c3ccc4oc5ccccc5c4c3-c3cc(-c4ccccc4)cc(-c4ccccc4)c3)cc2)cc1 ; CC1(C)c2ccccc2-c2ccc(N(c3ccc(-c4ccccc4)cc3)c3ccc4oc5ccccc5c4c3-c3cc(-c4ccccc4)cc(-c4ccccc4)c3)cc21 ; c1ccc(-c2ccc(N(c3ccc(-c4ccccc4)cc3)c3ccc4oc5ccccc5c4c3-c3cc(-c4ccccc4)cc(-c4ccccc4)c3)cc2)cc1]

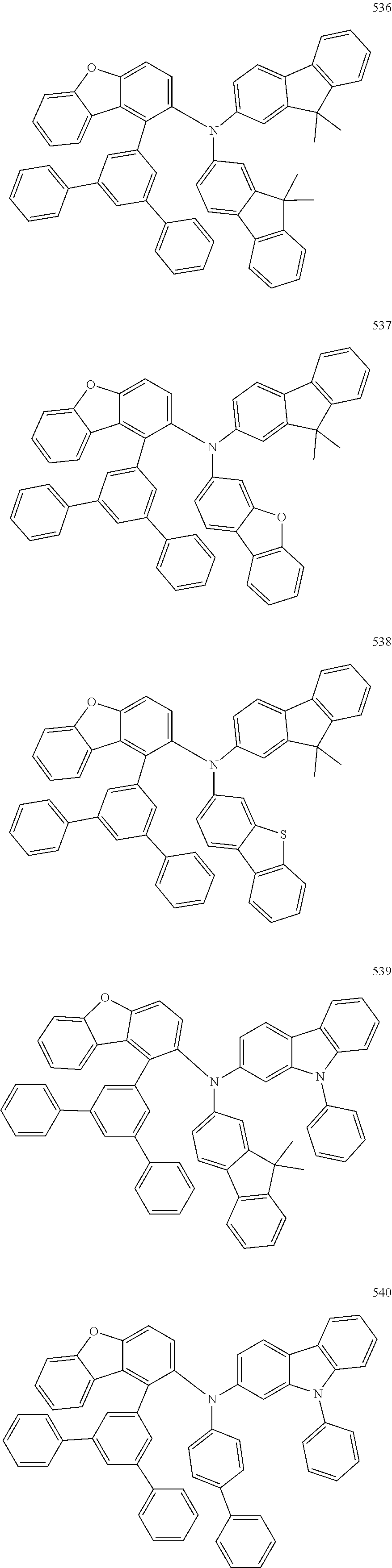

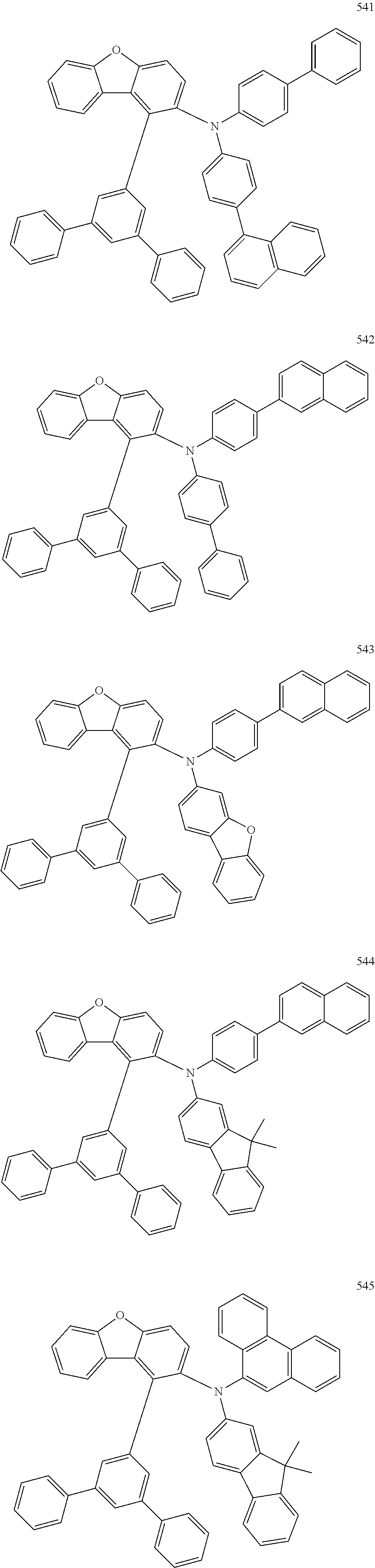

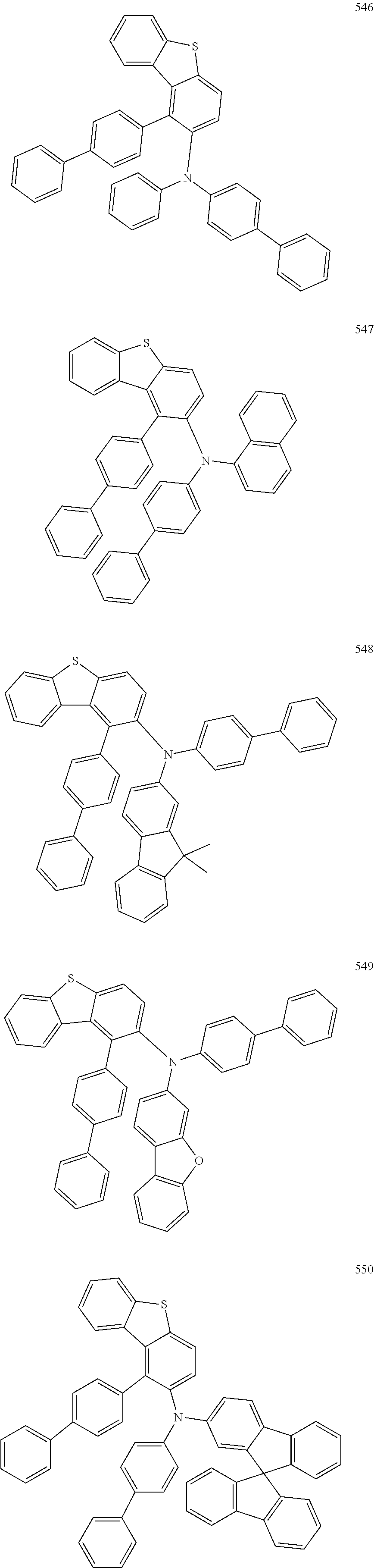 [SMILES: c1ccc(-c2ccc(-c3c(N(c4ccccc4)c4ccc(-c5ccccc5)cc4)ccc4sc5ccccc5c34)cc2)cc1 ; c1ccc(-c2ccc(-c3c(N(c4ccc(-c5ccccc5)cc4)c4cccc5ccccc45)ccc4sc5ccccc5c34)cc2)cc1 ; CC1(C)c2ccccc2-c2ccc(N(c3ccc(-c4ccccc4)cc3)c3ccc4sc5ccccc5c4c3-c3ccc(-c4ccccc4)cc3)cc21 ; c1ccc(-c2ccc(-c3c(N(c4ccc(-c5ccccc5)cc4)c4ccc5c(c4)oc4ccccc45)ccc4sc5ccccc5c34)cc2)cc1 ; c1ccc(-c2ccc(-c3c(N(c4ccc(-c5ccccc5)cc4)c4ccc5c(c4)C4(c6ccccc6-c6ccccc64)c4ccccc4-5)ccc4sc5ccccc5c34)cc2)cc1]

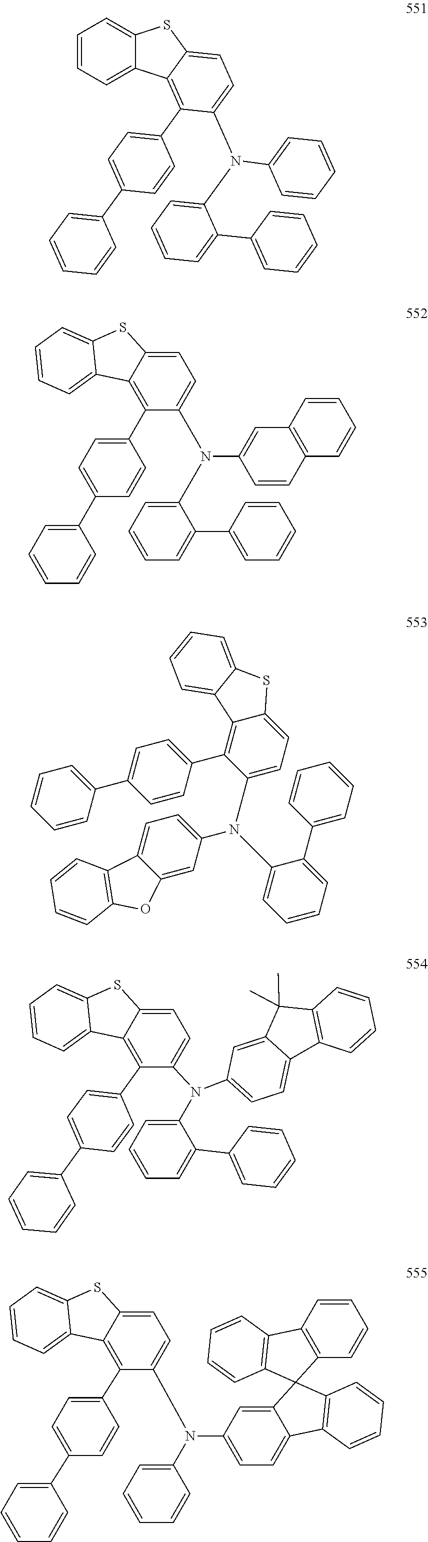 [SMILES: c1ccc(-c2ccc(-c3c(N(c4ccccc4)c4ccccc4-c4ccccc4)ccc4sc5ccccc5c34)cc2)cc1 ; c1ccc(-c2ccc(-c3c(N(c4ccc5ccccc5c4)c4ccccc4-c4ccccc4)ccc4sc5ccccc5c34)cc2)cc1 ; c1ccc(-c2ccc(-c3c(N(c4ccc5c(c4)oc4ccccc45)c4ccccc4-c4ccccc4)ccc4sc5ccccc5c34)cc2)cc1 ; CC1(C)c2ccccc2-c2ccc(N(c3ccccc3-c3ccccc3)c3ccc4sc5ccccc5c4c3-c3ccc(-c4ccccc4)cc3)cc21 ; c1ccc(-c2ccc(-c3c(N(c4ccccc4)c4ccc5c(c4)C4(c6ccccc6-c6ccccc64)c4ccccc4-5)ccc4sc5ccccc5c34)cc2)cc1]

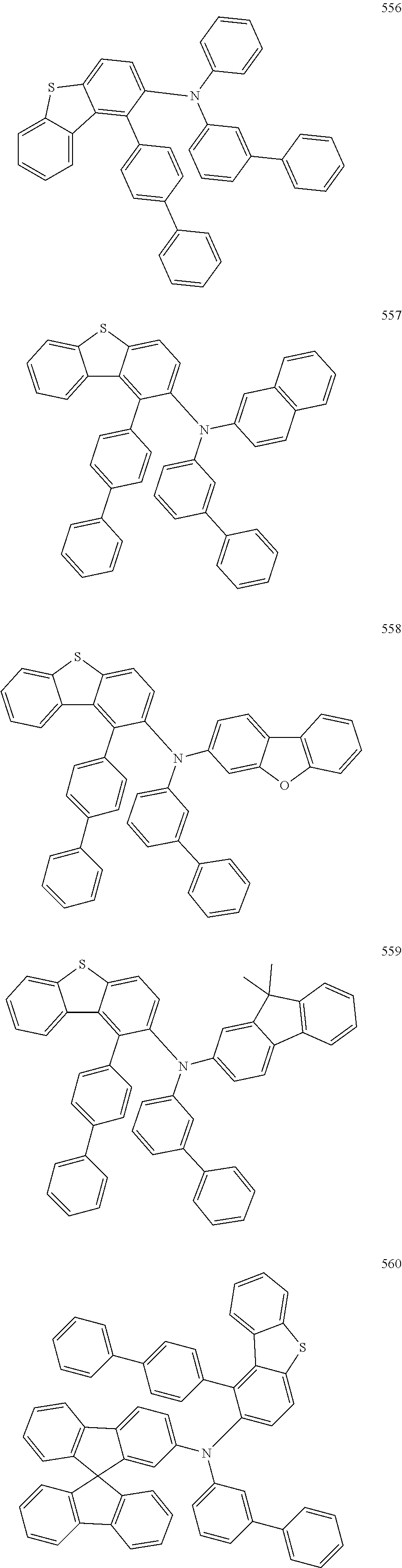 [SMILES: c1ccc(-c2ccc(-c3c(N(c4ccccc4)c4cccc(-c5ccccc5)c4)ccc4sc5ccccc5c34)cc2)cc1 ; c1ccc(-c2ccc(-c3c(N(c4cccc(-c5ccccc5)c4)c4ccc5ccccc5c4)ccc4sc5ccccc5c34)cc2)cc1 ; c1ccc(-c2ccc(-c3c(N(c4cccc(-c5ccccc5)c4)c4ccc5c(c4)oc4ccccc45)ccc4sc5ccccc5c34)cc2)cc1 ; CC1(C)c2ccccc2-c2ccc(N(c3cccc(-c4ccccc4)c3)c3ccc4sc5ccccc5c4c3-c3ccc(-c4ccccc4)cc3)cc21 ; c1ccc(-c2ccc(-c3c(N(c4cccc(-c5ccccc5)c4)c4ccc5c(c4)C4(c6ccccc6-c6ccccc64)c4ccccc4-5)ccc4sc5ccccc5c34)cc2)cc1]

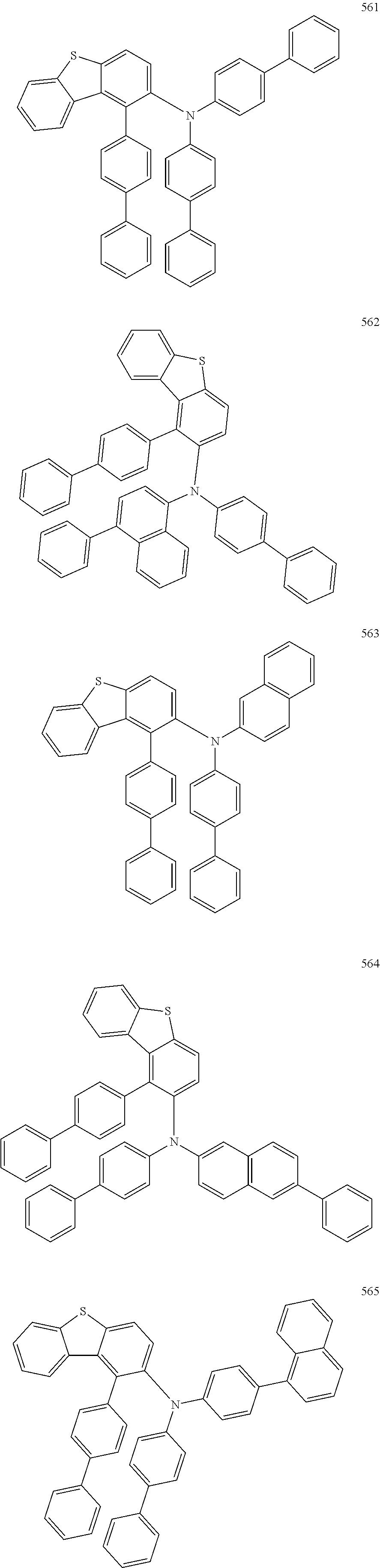 [SMILES: c1ccc(-c2ccc(-c3c(N(c4ccc(-c5ccccc5)cc4)c4ccc(-c5ccccc5)cc4)ccc4sc5ccccc5c34)cc2)cc1 ; c1ccc(-c2ccc(-c3c(N(c4ccc(-c5ccccc5)cc4)c4ccc(-c5ccccc5)c5ccccc45)ccc4sc5ccccc5c34)cc2)cc1 ; c1ccc(-c2ccc(-c3c(N(c4ccc(-c5ccccc5)cc4)c4ccc5ccccc5c4)ccc4sc5ccccc5c34)cc2)cc1 ; c1ccc(-c2ccc(-c3c(N(c4ccc(-c5ccccc5)cc4)c4ccc5cc(-c6ccccc6)ccc5c4)ccc4sc5ccccc5c34)cc2)cc1 ; c1ccc(-c2ccc(-c3c(N(c4ccc(-c5ccccc5)cc4)c4ccc(-c5cccc6ccccc56)cc4)ccc4sc5ccccc5c34)cc2)cc1]

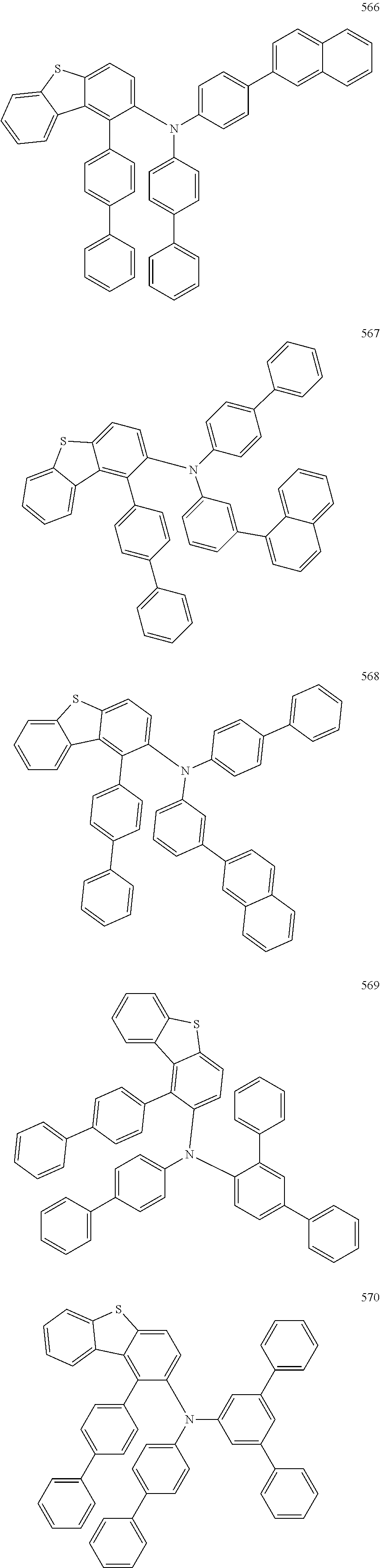 [SMILES: c1ccc(-c2ccc(-c3c(N(c4ccc(-c5ccccc5)cc4)c4ccc(-c5ccc6ccccc6c5)cc4)ccc4sc5ccccc5c34)cc2)cc1 ; c1ccc(-c2ccc(-c3c(N(c4ccc(-c5ccccc5)cc4)c4cccc(-c5cccc6ccccc56)c4)ccc4sc5ccccc5c34)cc2)cc1 ; c1ccc(-c2ccc(-c3c(N(c4ccc(-c5ccccc5)cc4)c4cccc(-c5ccc6ccccc6c5)c4)ccc4sc5ccccc5c34)cc2)cc1 ; c1ccc(-c2ccc(-c3c(N(c4ccc(-c5ccccc5)cc4)c4ccc(-c5ccccc5)cc4-c4ccccc4)ccc4sc5ccccc5c34)cc2)cc1 ; c1ccc(-c2ccc(-c3c(N(c4ccc(-c5ccccc5)cc4)c4cc(-c5ccccc5)cc(-c5ccccc5)c4)ccc4sc5ccccc5c34)cc2)cc1]

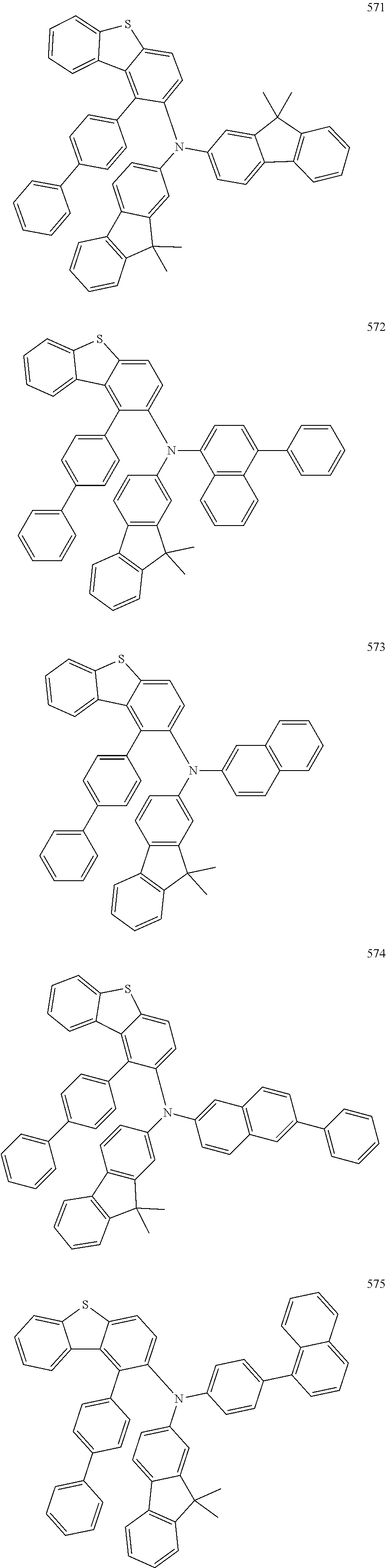 [SMILES: CC1(C)c2ccccc2-c2ccc(N(c3ccc4c(c3)C(C)(C)c3ccccc3-4)c3ccc4sc5ccccc5c4c3-c3ccc(-c4ccccc4)cc3)cc21 ; CC1(C)c2ccccc2-c2ccc(N(c3ccc4sc5ccccc5c4c3-c3ccc(-c4ccccc4)cc3)c3ccc(-c4ccccc4)c4ccccc34)cc21 ; CC1(C)c2ccccc2-c2ccc(N(c3ccc4ccccc4c3)c3ccc4sc5ccccc5c4c3-c3ccc(-c4ccccc4)cc3)cc21 ; CC1(C)c2ccccc2-c2ccc(N(c3ccc4cc(-c5ccccc5)ccc4c3)c3ccc4sc5ccccc5c4c3-c3ccc(-c4ccccc4)cc3)cc21 ; CC1(C)c2ccccc2-c2ccc(N(c3ccc(-c4cccc5ccccc45)cc3)c3ccc4sc5ccccc5c4c3-c3ccc(-c4ccccc4)cc3)cc21]

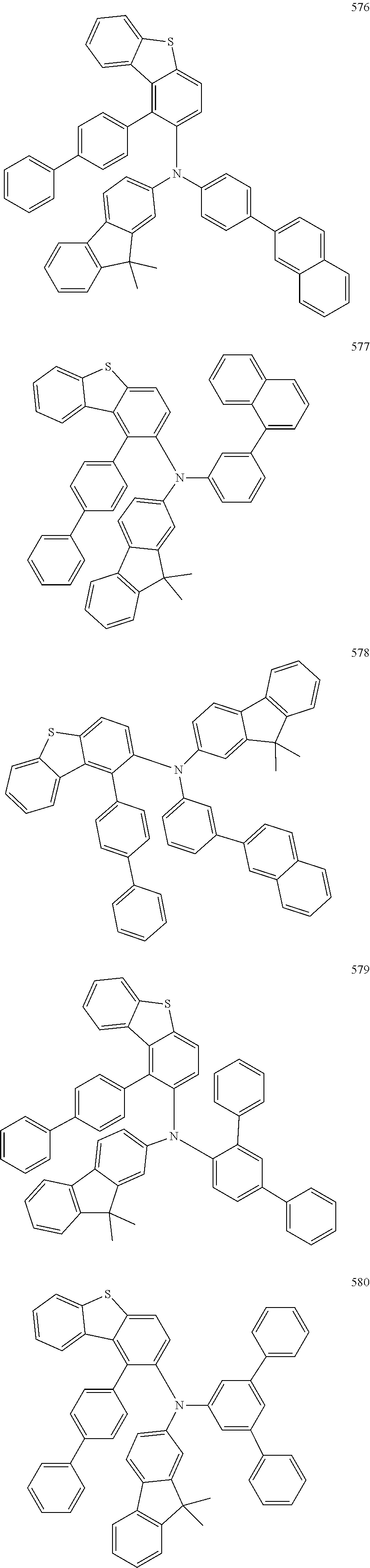 [SMILES: CC1(C)c2ccccc2-c2ccc(N(c3ccc(-c4ccc5ccccc5c4)cc3)c3ccc4sc5ccccc5c4c3-c3ccc(-c4ccccc4)cc3)cc21 ; CC1(C)c2ccccc2-c2ccc(N(c3cccc(-c4cccc5ccccc45)c3)c3ccc4sc5ccccc5c4c3-c3ccc(-c4ccccc4)cc3)cc21 ; CC1(C)c2ccccc2-c2ccc(N(c3cccc(-c4ccc5ccccc5c4)c3)c3ccc4sc5ccccc5c4c3-c3ccc(-c4ccccc4)cc3)cc21 ; CC1(C)c2ccccc2-c2ccc(N(c3ccc(-c4ccccc4)cc3-c3ccccc3)c3ccc4sc5ccccc5c4c3-c3ccc(-c4ccccc4)cc3)cc21 ; CC1(C)c2ccccc2-c2ccc(N(c3cc(-c4ccccc4)cc(-c4ccccc4)c3)c3ccc4sc5ccccc5c4c3-c3ccc(-c4ccccc4)cc3)cc21]

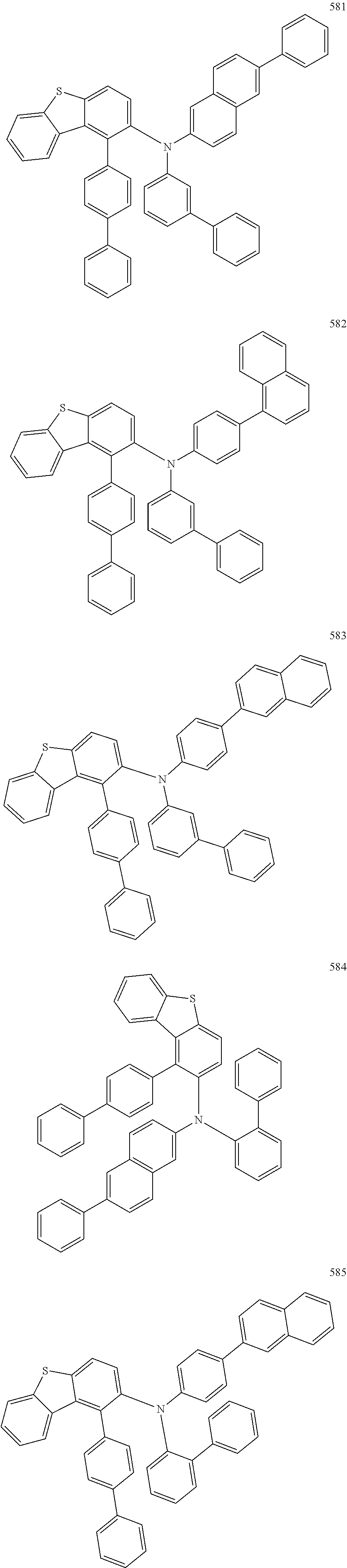 [SMILES: c1ccc(-c2ccc(-c3c(N(c4cccc(-c5ccccc5)c4)c4ccc5cc(-c6ccccc6)ccc5c4)ccc4sc5ccccc5c34)cc2)cc1 ; c1ccc(-c2ccc(-c3c(N(c4ccc(-c5cccc6ccccc56)cc4)c4cccc(-c5ccccc5)c4)ccc4sc5ccccc5c34)cc2)cc1 ; c1ccc(-c2ccc(-c3c(N(c4ccc(-c5ccc6ccccc6c5)cc4)c4cccc(-c5ccccc5)c4)ccc4sc5ccccc5c34)cc2)cc1 ; c1ccc(-c2ccc(-c3c(N(c4ccc5cc(-c6ccccc6)ccc5c4)c4ccccc4-c4ccccc4)ccc4sc5ccccc5c34)cc2)cc1 ; c1ccc(-c2ccc(-c3c(N(c4ccc(-c5ccc6ccccc6c5)cc4)c4ccccc4-c4ccccc4)ccc4sc5ccccc5c34)cc2)cc1]

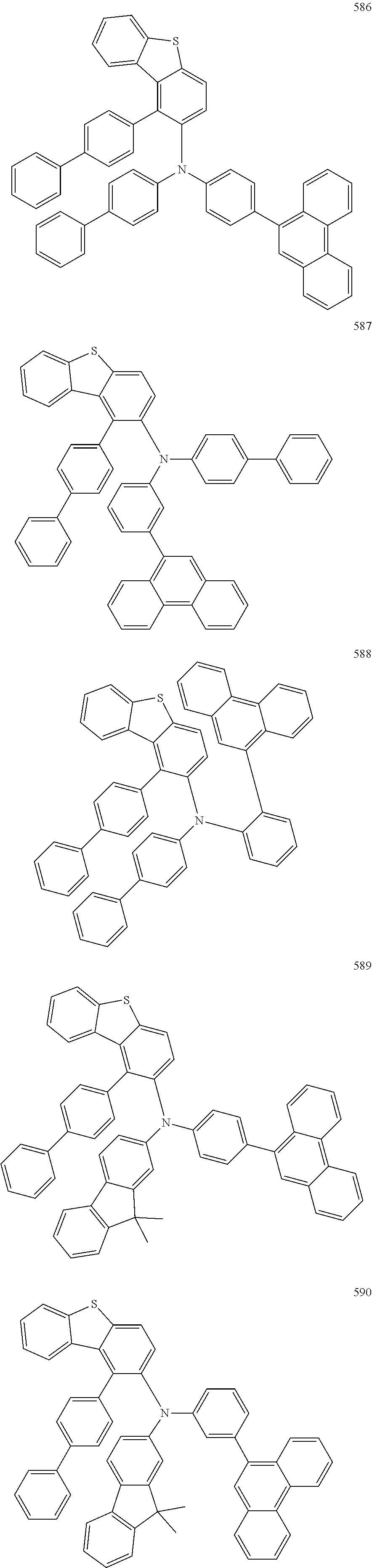

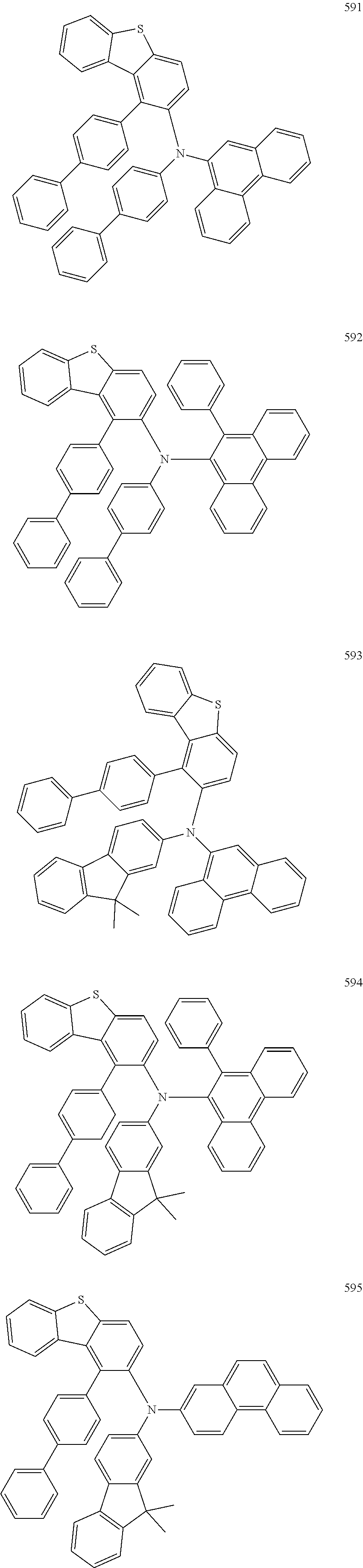 [SMILES: c1ccc(-c2ccc(-c3c(N(c4ccc(-c5ccccc5)cc4)c4cc5ccccc5c5ccccc45)ccc4sc5ccccc5c34)cc2)cc1 ; c1ccc(-c2ccc(-c3c(N(c4ccc(-c5ccccc5)cc4)c4c(-c5ccccc5)c5ccccc5c5ccccc45)ccc4sc5ccccc5c34)cc2)cc1 ; CC1(C)c2ccccc2-c2ccc(N(c3ccc4sc5ccccc5c4c3-c3ccc(-c4ccccc4)cc3)c3cc4ccccc4c4ccccc34)cc21 ; CC1(C)c2ccccc2-c2ccc(N(c3ccc4sc5ccccc5c4c3C3=CC=C(c4ccccc4)CC3)c3c(-c4ccccc4)c4ccccc4c4ccccc34)cc21 ; CC1(C)c2ccccc2-c2ccc(N(c3ccc4c(ccc5ccccc54)c3)c3ccc4sc5ccccc5c4c3-c3ccc(-c4ccccc4)cc3)cc21]

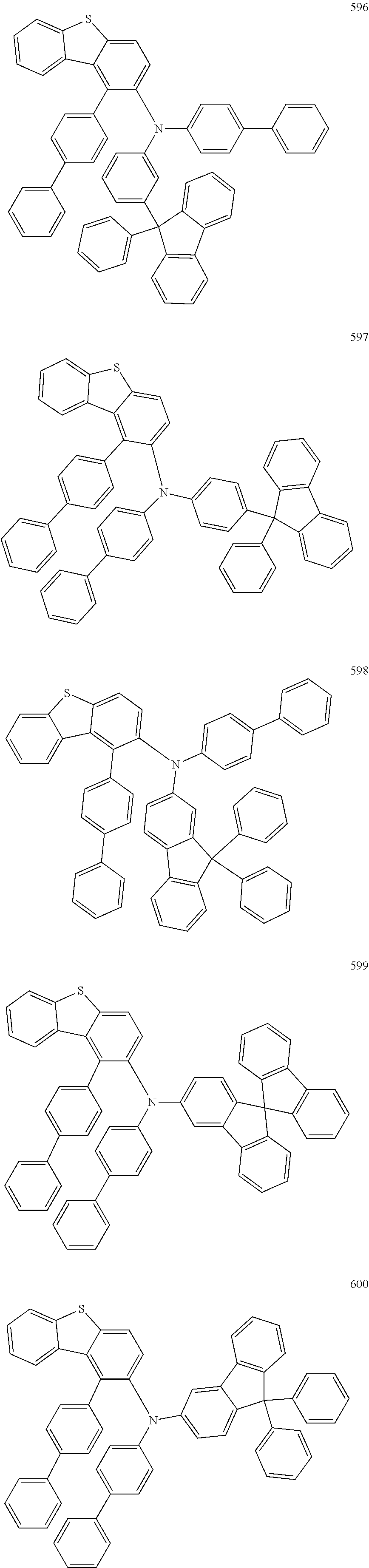

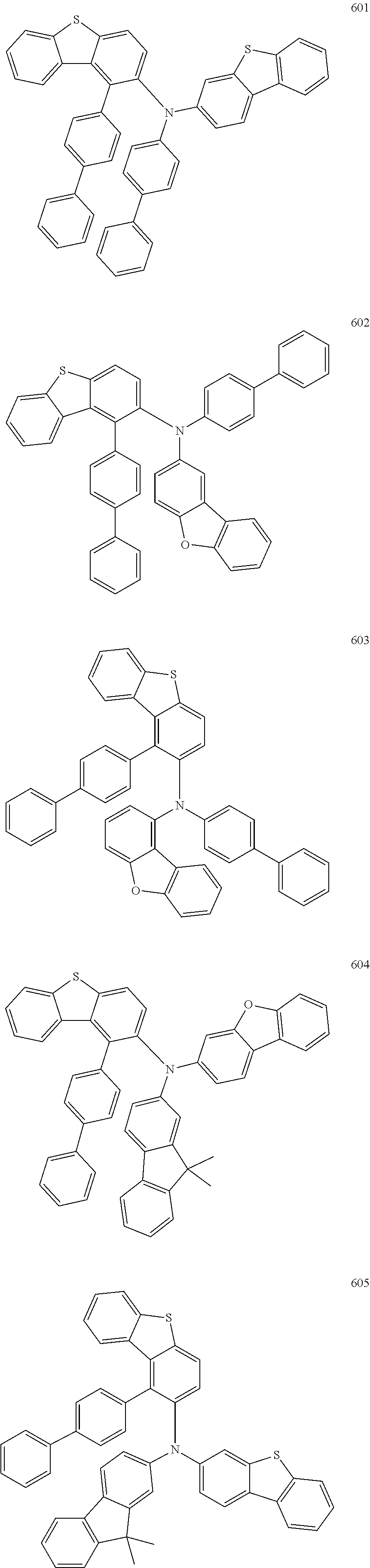 [SMILES: c1ccc(-c2ccc(-c3c(N(c4ccc(-c5ccccc5)cc4)c4ccc5c(c4)sc4ccccc45)ccc4sc5ccccc5c34)cc2)cc1 ; c1ccc(-c2ccc(-c3c(N(c4ccc(-c5ccccc5)cc4)c4ccc5oc6ccccc6c5c4)ccc4sc5ccccc5c34)cc2)cc1 ; c1ccc(-c2ccc(-c3c(N(c4ccc(-c5ccccc5)cc4)c4cccc5oc6ccccc6c45)ccc4sc5ccccc5c34)cc2)cc1 ; CC1(C)c2ccccc2-c2ccc(N(c3ccc4c(c3)oc3ccccc34)c3ccc4sc5ccccc5c4c3-c3ccc(-c4ccccc4)cc3)cc21 ; CC1(C)c2ccccc2-c2ccc(N(c3ccc4c(c3)sc3ccccc34)c3ccc4sc5ccccc5c4c3-c3ccc(-c4ccccc4)cc3)cc21]

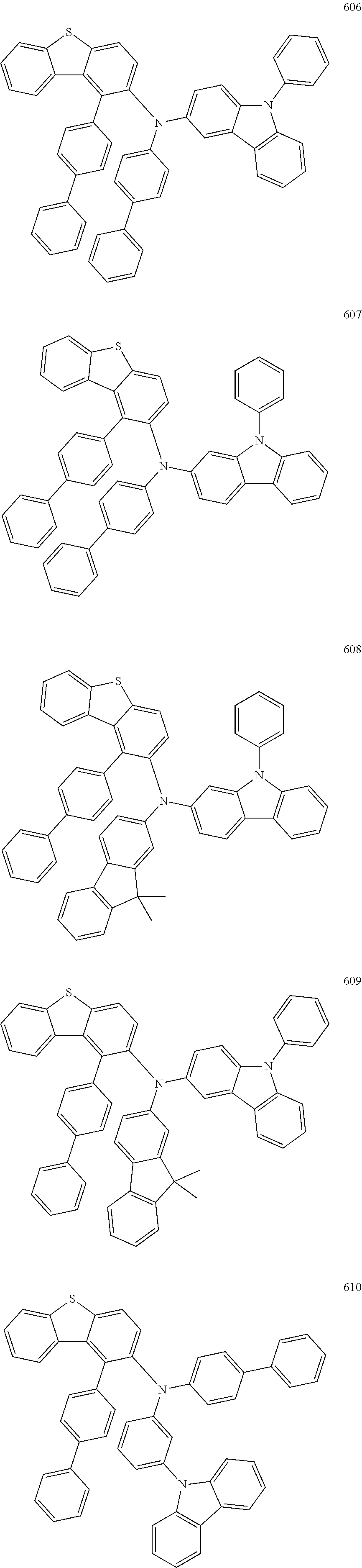 [SMILES: c1ccc(-c2ccc(-c3c(N(c4ccc(-c5ccccc5)cc4)c4ccc5c(c4)c4ccccc4n5-c4ccccc4)ccc4sc5ccccc5c34)cc2)cc1 ; c1ccc(-c2ccc(-c3c(N(c4ccc(-c5ccccc5)cc4)c4ccc5c6ccccc6n(-c6ccccc6)c5c4)ccc4sc5ccccc5c34)cc2)cc1 ; CC1(C)c2ccccc2-c2ccc(N(c3ccc4c5ccccc5n(-c5ccccc5)c4c3)c3ccc4sc5ccccc5c4c3-c3ccc(-c4ccccc4)cc3)cc21 ; CC1(C)c2ccccc2-c2ccc(N(c3ccc4c(c3)c3ccccc3n4-c3ccccc3)c3ccc4sc5ccccc5c4c3-c3ccc(-c4ccccc4)cc3)cc21 ; c1ccc(-c2ccc(-c3c(N(c4ccc(-c5ccccc5)cc4)c4cccc(-n5c6ccccc6c6ccccc65)c4)ccc4sc5ccccc5c34)cc2)cc1]

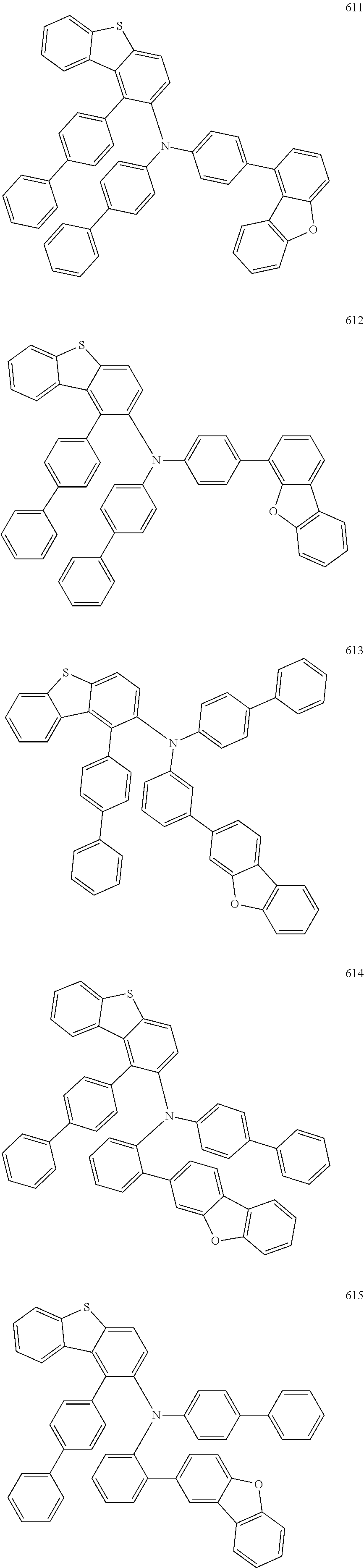 [SMILES: c1ccc(-c2ccc(-c3c(N(c4ccc(-c5ccccc5)cc4)c4ccc(-c5cccc6oc7ccccc7c56)cc4)ccc4sc5ccccc5c34)cc2)cc1 ; c1ccc(-c2ccc(-c3c(N(c4ccc(-c5ccccc5)cc4)c4ccc(-c5cccc6c5oc5ccccc56)cc4)ccc4sc5ccccc5c34)cc2)cc1 ; c1ccc(-c2ccc(-c3c(N(c4ccc(-c5ccccc5)cc4)c4cccc(-c5ccc6c(c5)oc5ccccc56)c4)ccc4sc5ccccc5c34)cc2)cc1 ; c1ccc(-c2ccc(-c3c(N(c4ccc(-c5ccccc5)cc4)c4ccccc4-c4ccc5c(c4)oc4ccccc45)ccc4sc5ccccc5c34)cc2)cc1 ; c1ccc(-c2ccc(-c3c(N(c4ccc(-c5ccccc5)cc4)c4ccccc4-c4ccc5oc6ccccc6c5c4)ccc4sc5ccccc5c34)cc2)cc1]

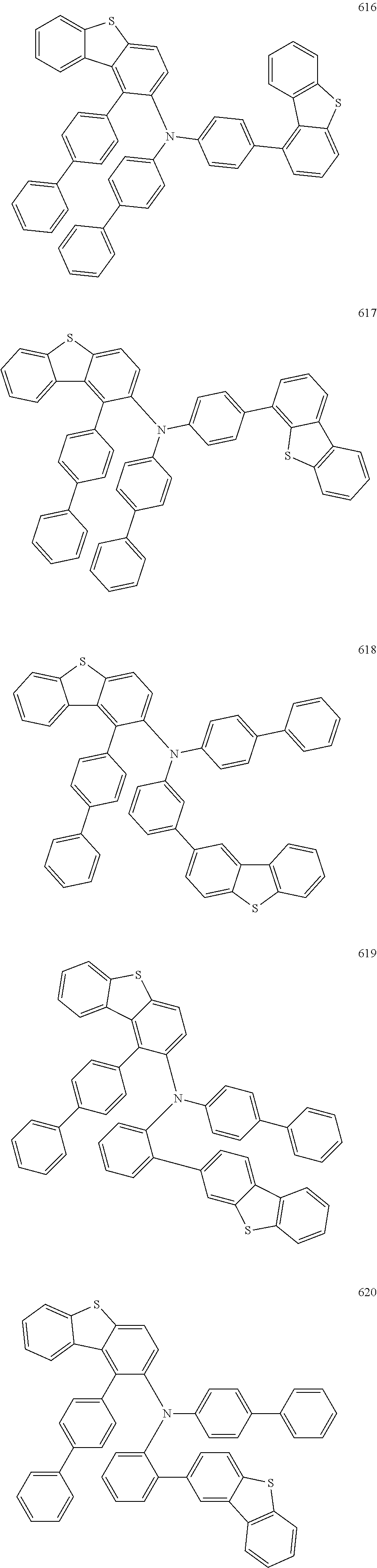 [SMILES: c1ccc(-c2ccc(-c3c(N(c4ccc(-c5ccccc5)cc4)c4ccc(-c5cccc6sc7ccccc7c56)cc4)ccc4sc5ccccc5c34)cc2)cc1 ; c1ccc(-c2ccc(-c3c(N(c4ccc(-c5ccccc5)cc4)c4ccc(-c5cccc6c5sc5ccccc56)cc4)ccc4sc5ccccc5c34)cc2)cc1 ; c1ccc(-c2ccc(-c3c(N(c4ccc(-c5ccccc5)cc4)c4cccc(-c5ccc6sc7ccccc7c6c5)c4)ccc4sc5ccccc5c34)cc2)cc1 ; c1ccc(-c2ccc(-c3c(N(c4ccc(-c5ccccc5)cc4)c4ccccc4-c4ccc5c(c4)sc4ccccc45)ccc4sc5ccccc5c34)cc2)cc1 ; c1ccc(-c2ccc(-c3c(N(c4ccc(-c5ccccc5)cc4)c4ccccc4-c4ccc5sc6ccccc6c5c4)ccc4sc5ccccc5c34)cc2)cc1]

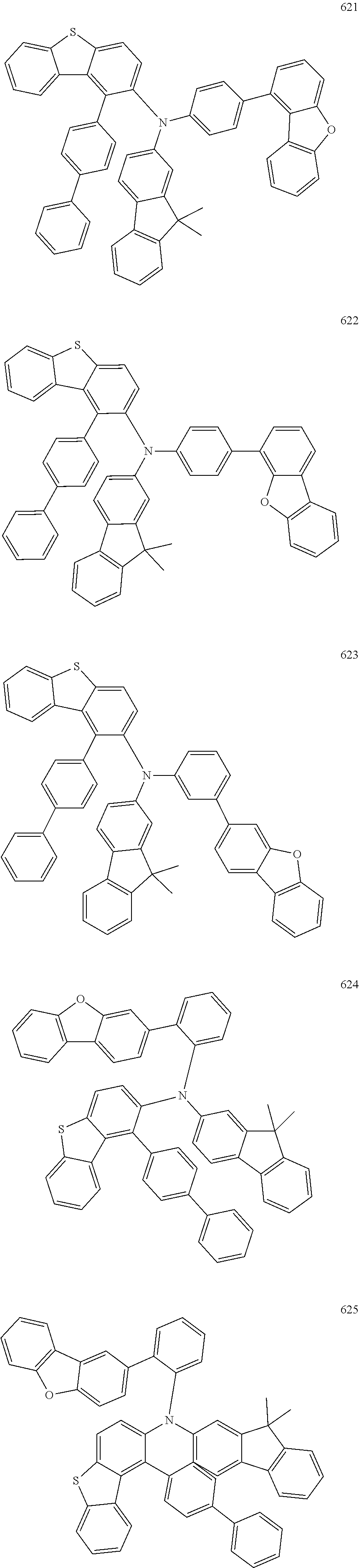

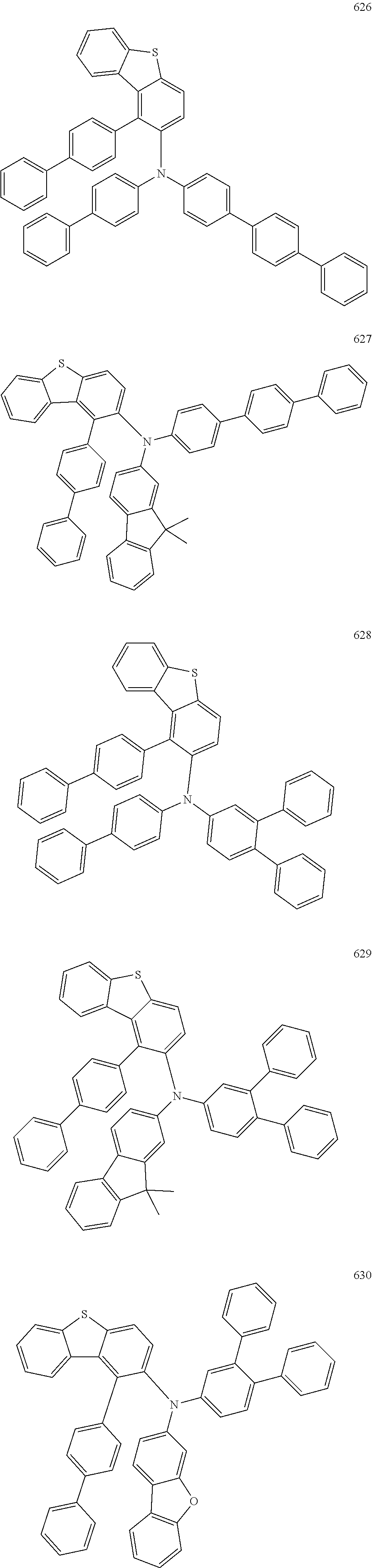 [SMILES: c1ccc(-c2ccc(-c3ccc(N(c4ccc(-c5ccccc5)cc4)c4ccc5sc6ccccc6c5c4-c4ccc(-c5ccccc5)cc4)cc3)cc2)cc1 ; CC1(C)c2ccccc2-c2ccc(N(c3ccc(-c4ccc(-c5ccccc5)cc4)cc3)c3ccc4sc5ccccc5c4c3-c3ccc(-c4ccccc4)cc3)cc21 ; c1ccc(-c2ccc(-c3c(N(c4ccc(-c5ccccc5)cc4)c4ccc(-c5ccccc5)c(-c5ccccc5)c4)ccc4sc5ccccc5c34)cc2)cc1 ; CC1(C)c2ccccc2-c2ccc(N(c3ccc(-c4ccccc4)c(-c4ccccc4)c3)c3ccc4sc5ccccc5c4c3-c3ccc(-c4ccccc4)cc3)cc21 ; c1ccc(-c2ccc(-c3c(N(c4ccc(-c5ccccc5)c(-c5ccccc5)c4)c4ccc5c(c4)oc4ccccc45)ccc4sc5ccccc5c34)cc2)cc1]

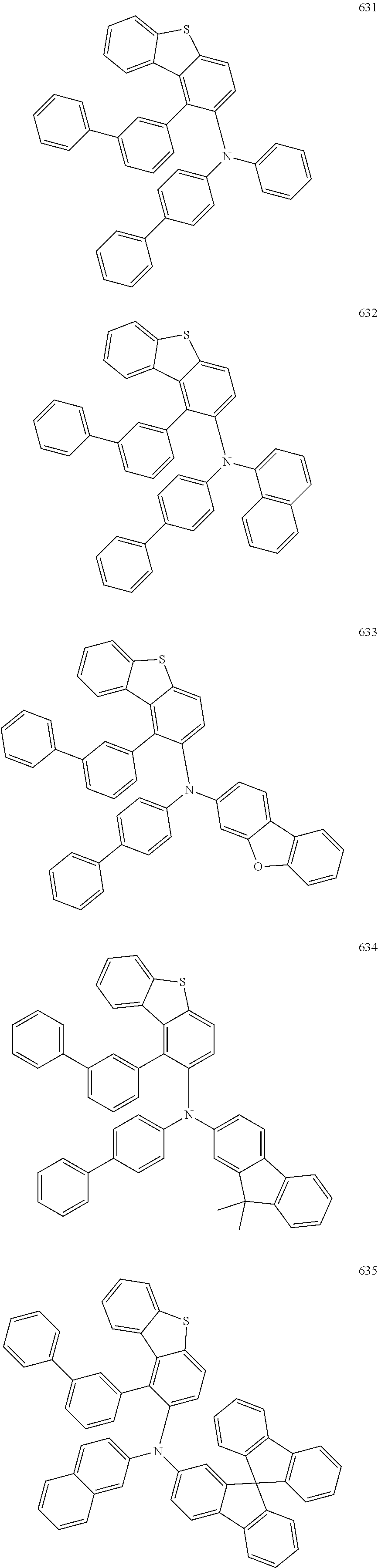

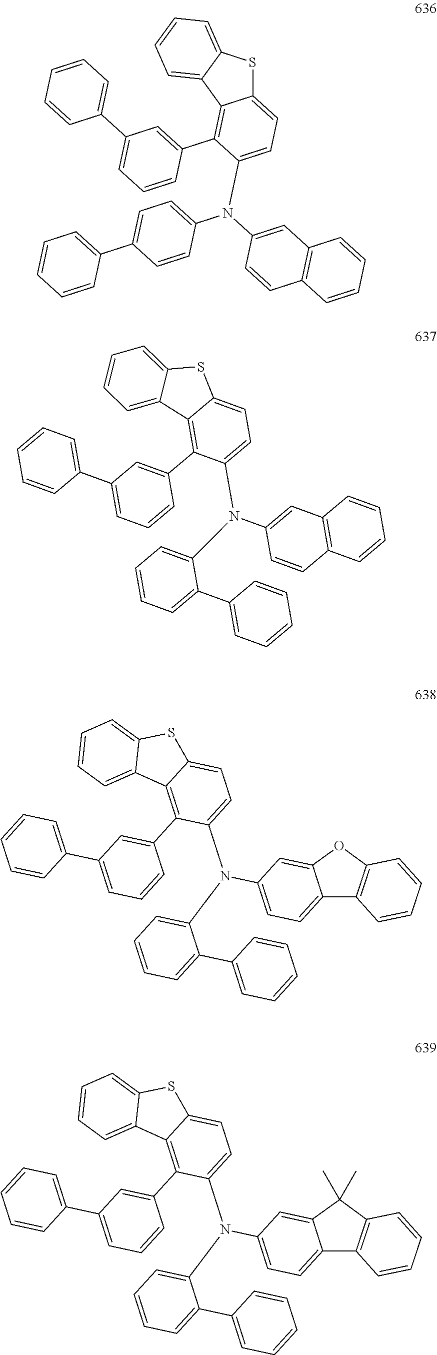 [SMILES: c1ccc(-c2ccc(N(c3ccc4ccccc4c3)c3ccc4sc5ccccc5c4c3-c3cccc(-c4ccccc4)c3)cc2)cc1 ; c1ccc(-c2cccc(-c3c(N(c4ccc5ccccc5c4)c4ccccc4-c4ccccc4)ccc4sc5ccccc5c34)c2)cc1 ; c1ccc(-c2cccc(-c3c(N(c4ccc5c(c4)oc4ccccc45)c4ccccc4-c4ccccc4)ccc4sc5ccccc5c34)c2)cc1 ; CC1(C)c2ccccc2-c2ccc(N(c3ccccc3-c3ccccc3)c3ccc4sc5ccccc5c4c3-c3cccc(-c4ccccc4)c3)cc21]

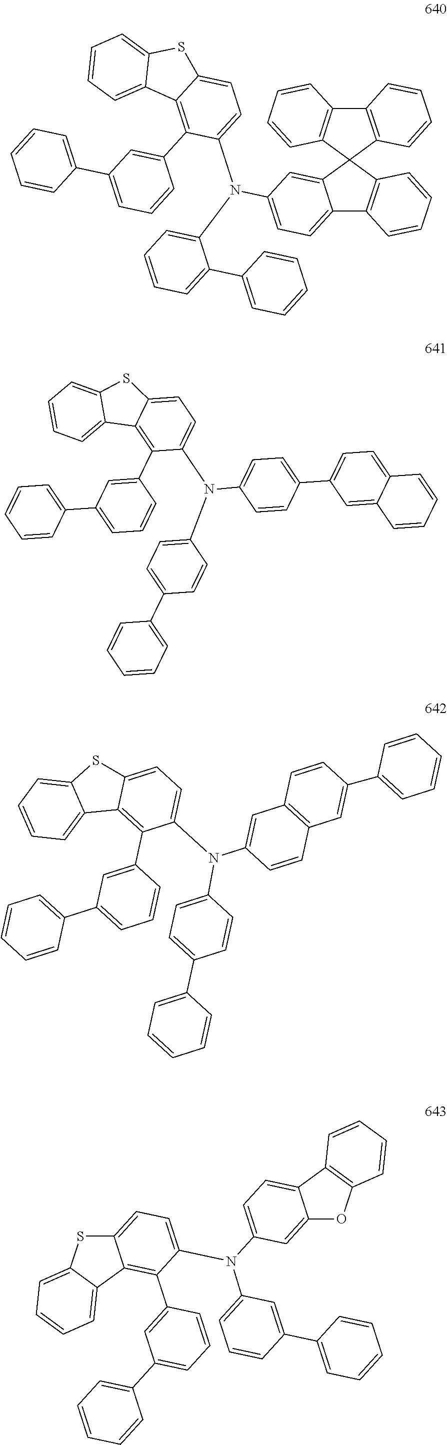 [SMILES: c1ccc(-c2cccc(-c3c(N(c4ccc5c(c4)C4(c6ccccc6-c6ccccc64)c4ccccc4-5)c4ccccc4-c4ccccc4)ccc4sc5ccccc5c34)c2)cc1 ; c1ccc(-c2ccc(N(c3ccc(-c4ccc5ccccc5c4)cc3)c3ccc4sc5ccccc5c4c3-c3cccc(-c4ccccc4)c3)cc2)cc1 ; c1ccc(-c2ccc(N(c3ccc4cc(-c5ccccc5)ccc4c3)c3ccc4sc5ccccc5c4c3-c3cccc(-c4ccccc4)c3)cc2)cc1 ; c1ccc(-c2cccc(-c3c(N(c4cccc(-c5ccccc5)c4)c4ccc5c(c4)oc4ccccc45)ccc4sc5ccccc5c34)c2)cc1]

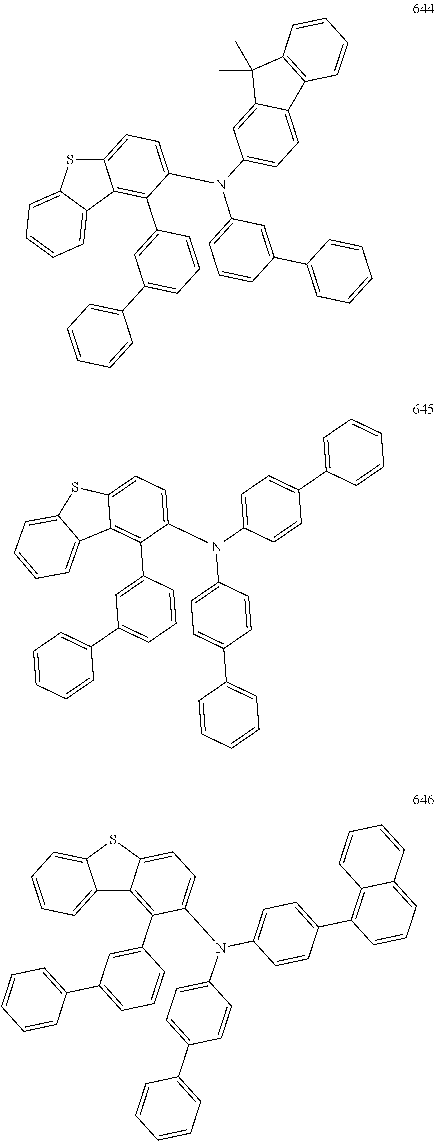 [SMILES: CC1(C)c2ccccc2-c2ccc(N(c3cccc(-c4ccccc4)c3)c3ccc4sc5ccccc5c4c3-c3cccc(-c4ccccc4)c3)cc21 ; c1ccc(-c2ccc(N(c3ccc(-c4ccccc4)cc3)c3ccc4sc5ccccc5c4c3-c3cccc(-c4ccccc4)c3)cc2)cc1 ; c1ccc(-c2ccc(N(c3ccc(-c4cccc5ccccc45)cc3)c3ccc4sc5ccccc5c4c3-c3cccc(-c4ccccc4)c3)cc2)cc1]

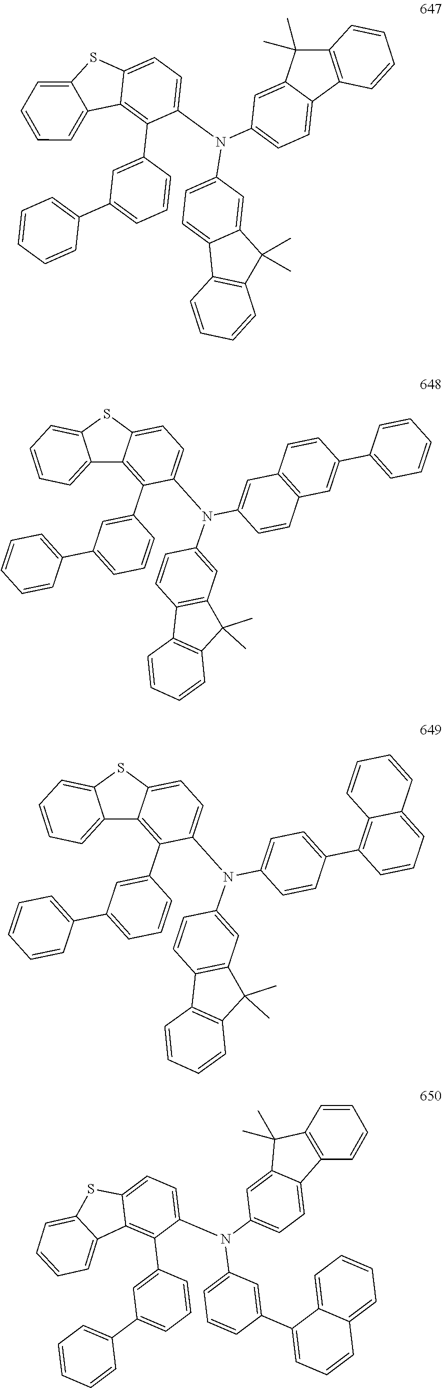 [SMILES: CC1(C)c2ccccc2-c2ccc(N(c3ccc4c(c3)C(C)(C)c3ccccc3-4)c3ccc4sc5ccccc5c4c3-c3cccc(-c4ccccc4)c3)cc21 ; CC1(C)c2ccccc2-c2ccc(N(c3ccc4cc(-c5ccccc5)ccc4c3)c3ccc4sc5ccccc5c4c3-c3cccc(-c4ccccc4)c3)cc21 ; CC1(C)c2ccccc2-c2ccc(N(c3ccc(-c4cccc5ccccc45)cc3)c3ccc4sc5ccccc5c4c3-c3cccc(-c4ccccc4)c3)cc21 ; CC1(C)c2ccccc2-c2ccc(N(c3cccc(-c4cccc5ccccc45)c3)c3ccc4sc5ccccc5c4c3-c3cccc(-c4ccccc4)c3)cc21]

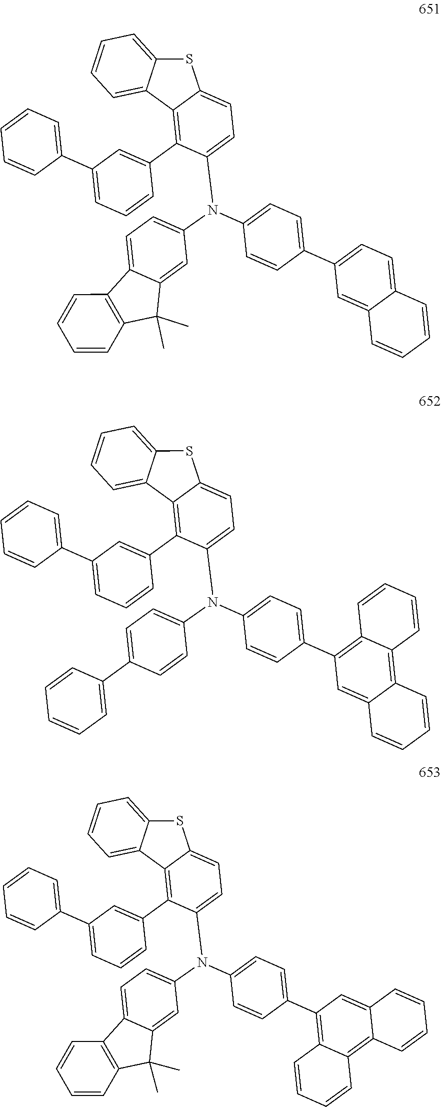 [SMILES: CC1(C)c2ccccc2-c2ccc(N(c3ccc(-c4ccc5ccccc5c4)cc3)c3ccc4sc5ccccc5c4c3-c3cccc(-c4ccccc4)c3)cc21 ; c1ccc(-c2ccc(N(c3ccc(-c4cc5ccccc5c5ccccc45)cc3)c3ccc4sc5ccccc5c4c3-c3cccc(-c4ccccc4)c3)cc2)cc1 ; CC1(C)c2ccccc2-c2ccc(N(c3ccc(-c4cc5ccccc5c5ccccc45)cc3)c3ccc4sc5ccccc5c4c3-c3cccc(-c4ccccc4)c3)cc21]

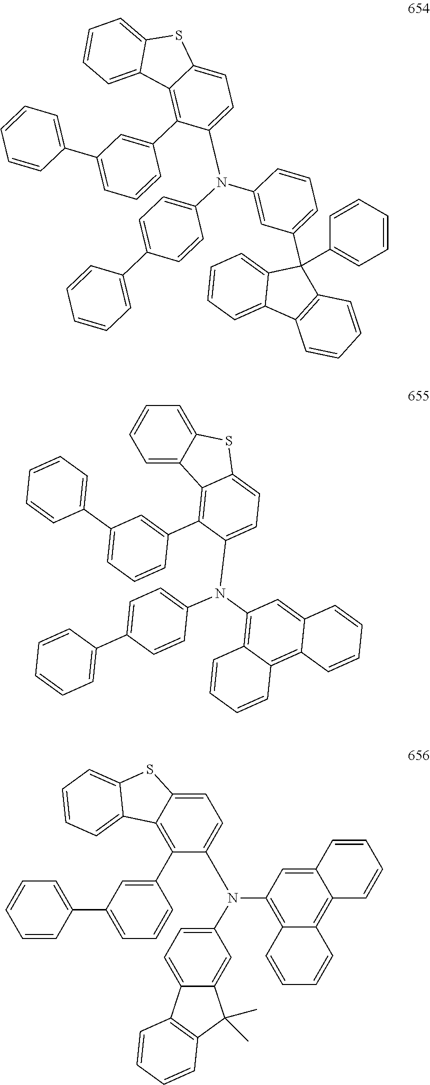 [SMILES: c1ccc(-c2ccc(N(c3cccc(C4(c5ccccc5)c5ccccc5-c5ccccc54)c3)c3ccc4sc5ccccc5c4c3-c3cccc(-c4ccccc4)c3)cc2)cc1 ; c1ccc(-c2ccc(N(c3ccc4sc5ccccc5c4c3-c3cccc(-c4ccccc4)c3)c3cc4ccccc4c4ccccc34)cc2)cc1 ; CC1(C)c2ccccc2-c2ccc(N(c3ccc4sc5ccccc5c4c3-c3cccc(-c4ccccc4)c3)c3cc4ccccc4c4ccccc34)cc21]

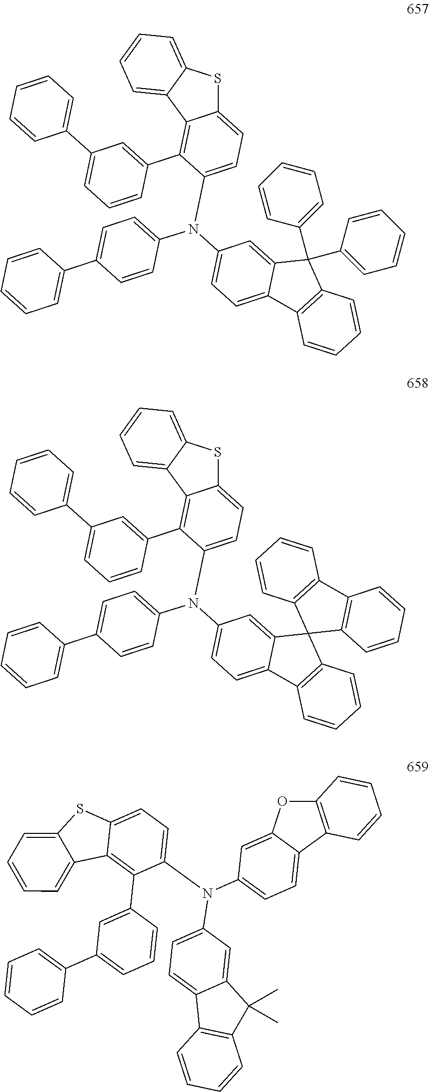 [SMILES: c1ccc(-c2ccc(N(c3ccc4c(c3)C(c3ccccc3)(c3ccccc3)c3ccccc3-4)c3ccc4sc5ccccc5c4c3-c3cccc(-c4ccccc4)c3)cc2)cc1 ; c1ccc(-c2ccc(N(c3ccc4c(c3)C3(c5ccccc5-c5ccccc53)c3ccccc3-4)c3ccc4sc5ccccc5c4c3-c3cccc(-c4ccccc4)c3)cc2)cc1 ; CC1(C)c2ccccc2-c2ccc(N(c3ccc4c(c3)oc3ccccc34)c3ccc4sc5ccccc5c4c3-c3cccc(-c4ccccc4)c3)cc21]

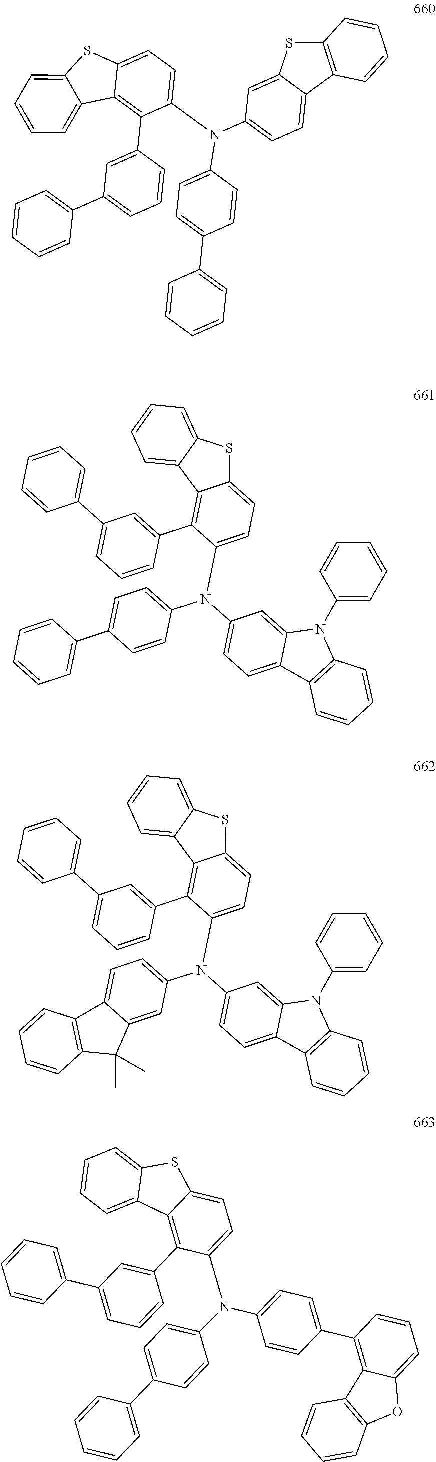 [SMILES: c1ccc(-c2ccc(N(c3ccc4c(c3)sc3ccccc34)c3ccc4sc5ccccc5c4c3-c3cccc(-c4ccccc4)c3)cc2)cc1 ; c1ccc(-c2ccc(N(c3ccc4c5ccccc5n(-c5ccccc5)c4c3)c3ccc4sc5ccccc5c4c3-c3cccc(-c4ccccc4)c3)cc2)cc1 ; CC1(C)c2ccccc2-c2ccc(N(c3ccc4c5ccccc5n(-c5ccccc5)c4c3)c3ccc4sc5ccccc5c4c3-c3cccc(-c4ccccc4)c3)cc21 ; c1ccc(-c2ccc(N(c3ccc(-c4cccc5oc6ccccc6c45)cc3)c3ccc4sc5ccccc5c4c3-c3cccc(-c4ccccc4)c3)cc2)cc1]

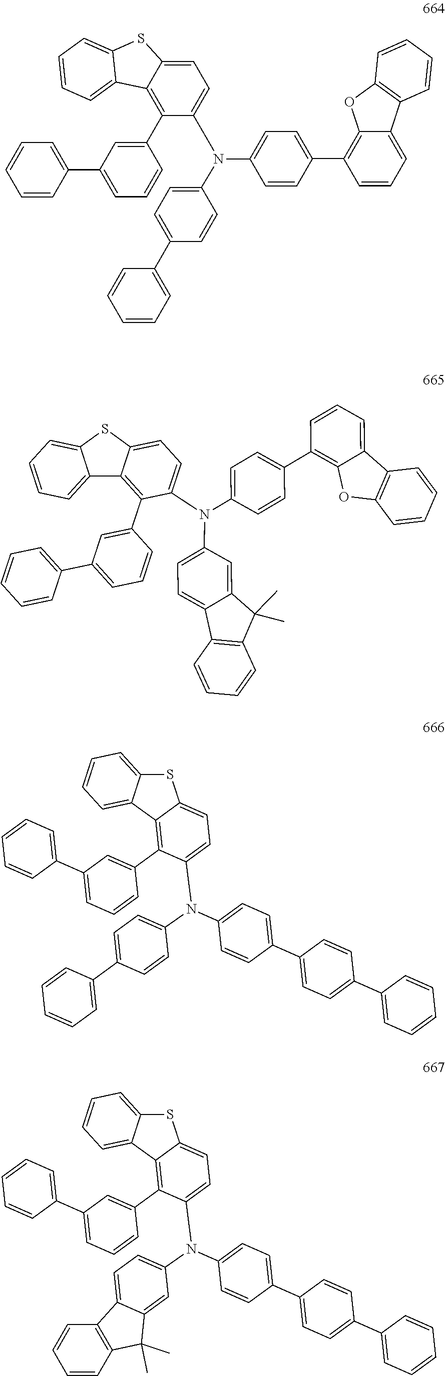 [SMILES: c1ccc(-c2ccc(N(c3ccc(-c4cccc5c4oc4ccccc45)cc3)c3ccc4sc5ccccc5c4c3-c3cccc(-c4ccccc4)c3)cc2)cc1 ; CC1(C)c2ccccc2-c2ccc(N(c3ccc(-c4cccc5c4oc4ccccc45)cc3)c3ccc4sc5ccccc5c4c3-c3cccc(-c4ccccc4)c3)cc21 ; c1ccc(-c2ccc(-c3ccc(N(c4ccc(-c5ccccc5)cc4)c4ccc5sc6ccccc6c5c4-c4cccc(-c5ccccc5)c4)cc3)cc2)cc1 ; CC1(C)c2ccccc2-c2ccc(N(c3ccc(-c4ccc(-c5ccccc5)cc4)cc3)c3ccc4sc5ccccc5c4c3-c3cccc(-c4ccccc4)c3)cc21]

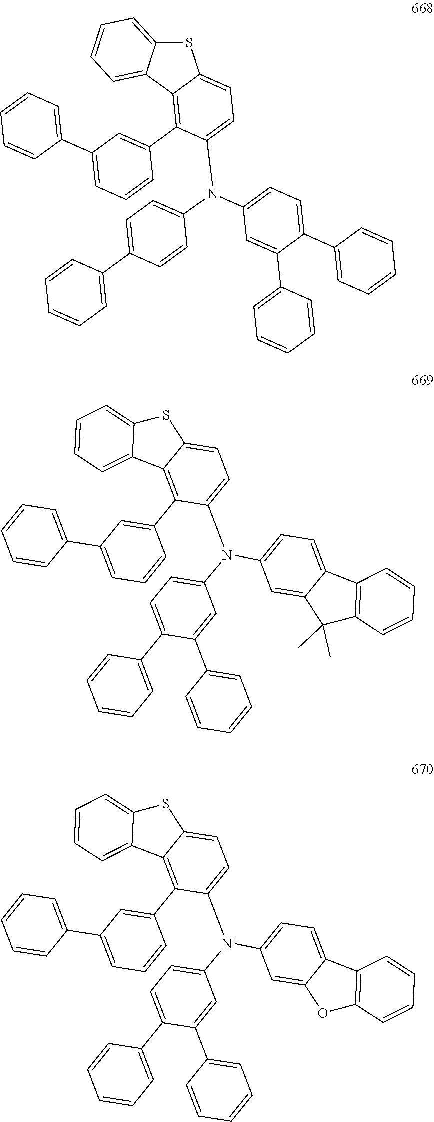 [SMILES: c1ccc(-c2ccc(N(c3ccc(-c4ccccc4)c(-c4ccccc4)c3)c3ccc4sc5ccccc5c4c3-c3cccc(-c4ccccc4)c3)cc2)cc1 ; CC1(C)c2ccccc2-c2ccc(N(c3ccc(-c4ccccc4)c(-c4ccccc4)c3)c3ccc4sc5ccccc5c4c3-c3cccc(-c4ccccc4)c3)cc21 ; c1ccc(-c2cccc(-c3c(N(c4ccc(-c5ccccc5)c(-c5ccccc5)c4)c4ccc5c(c4)oc4ccccc45)ccc4sc5ccccc5c34)c2)cc1]

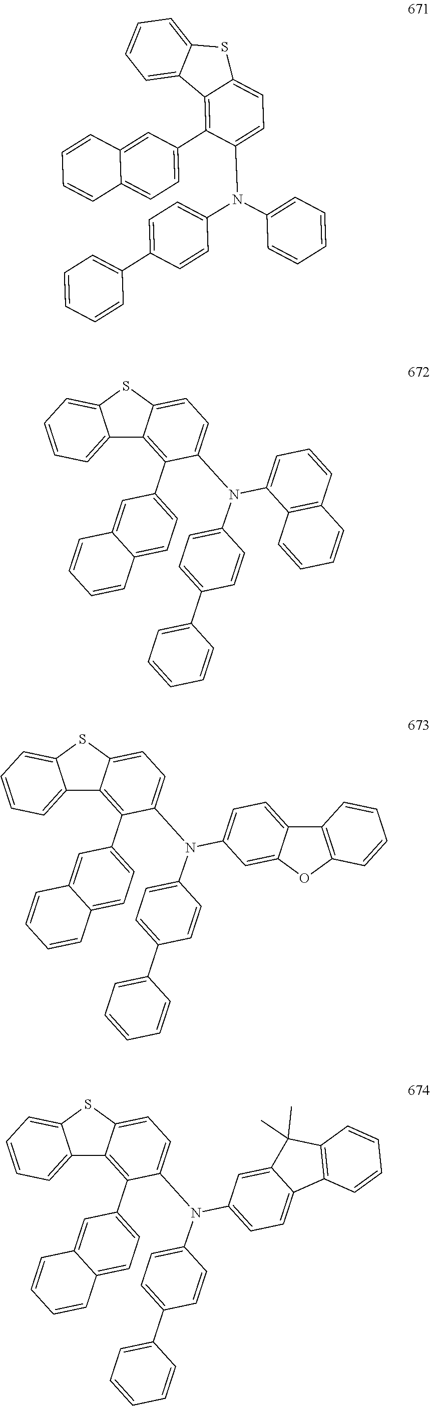 [SMILES: c1ccc(-c2ccc(N(c3ccccc3)c3ccc4sc5ccccc5c4c3-c3ccc4ccccc4c3)cc2)cc1 ; c1ccc(-c2ccc(N(c3ccc4sc5ccccc5c4c3-c3ccc4ccccc4c3)c3cccc4ccccc34)cc2)cc1 ; c1ccc(-c2ccc(N(c3ccc4c(c3)oc3ccccc34)c3ccc4sc5ccccc5c4c3-c3ccc4ccccc4c3)cc2)cc1 ; CC1(C)c2ccccc2-c2ccc(N(c3ccc(-c4ccccc4)cc3)c3ccc4sc5ccccc5c4c3-c3ccc4ccccc4c3)cc21]

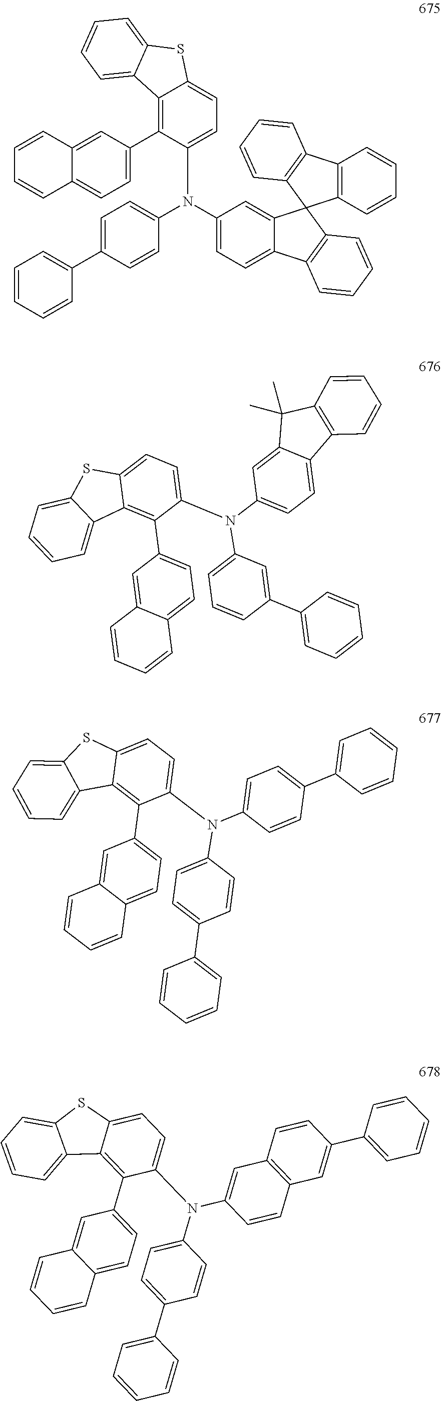 [SMILES: c1ccc(-c2ccc(N(c3ccc4c(c3)C3(c5ccccc5-c5ccccc53)c3ccccc3-4)c3ccc4sc5ccccc5c4c3-c3ccc4ccccc4c3)cc2)cc1 ; CC1(C)c2ccccc2-c2ccc(N(c3cccc(-c4ccccc4)c3)c3ccc4sc5ccccc5c4c3-c3ccc4ccccc4c3)cc21 ; c1ccc(-c2ccc(N(c3ccc(-c4ccccc4)cc3)c3ccc4sc5ccccc5c4c3-c3ccc4ccccc4c3)cc2)cc1 ; c1ccc(-c2ccc(N(c3ccc4cc(-c5ccccc5)ccc4c3)c3ccc4sc5ccccc5c4c3-c3ccc4ccccc4c3)cc2)cc1]

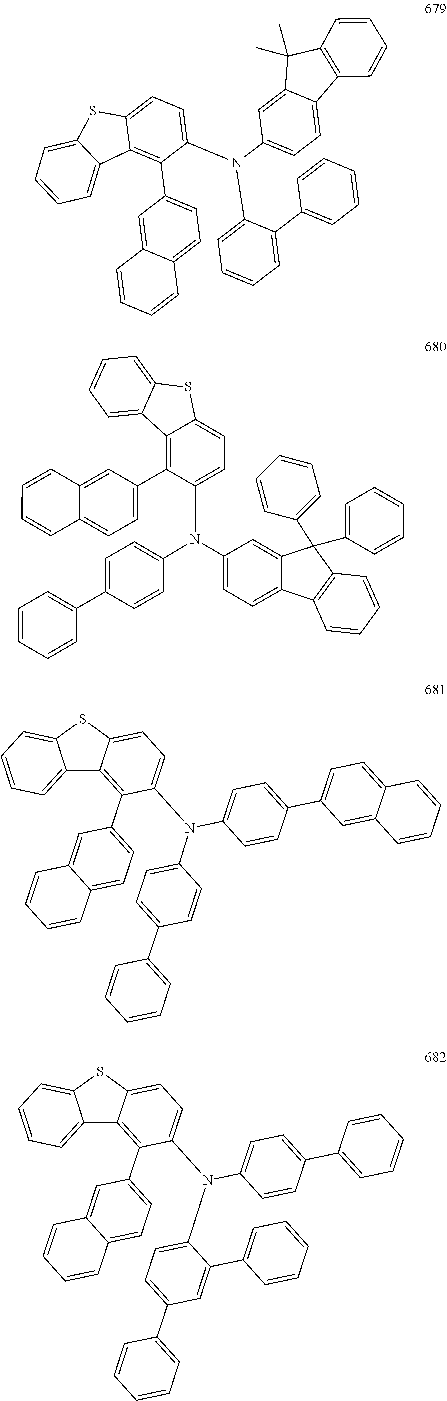

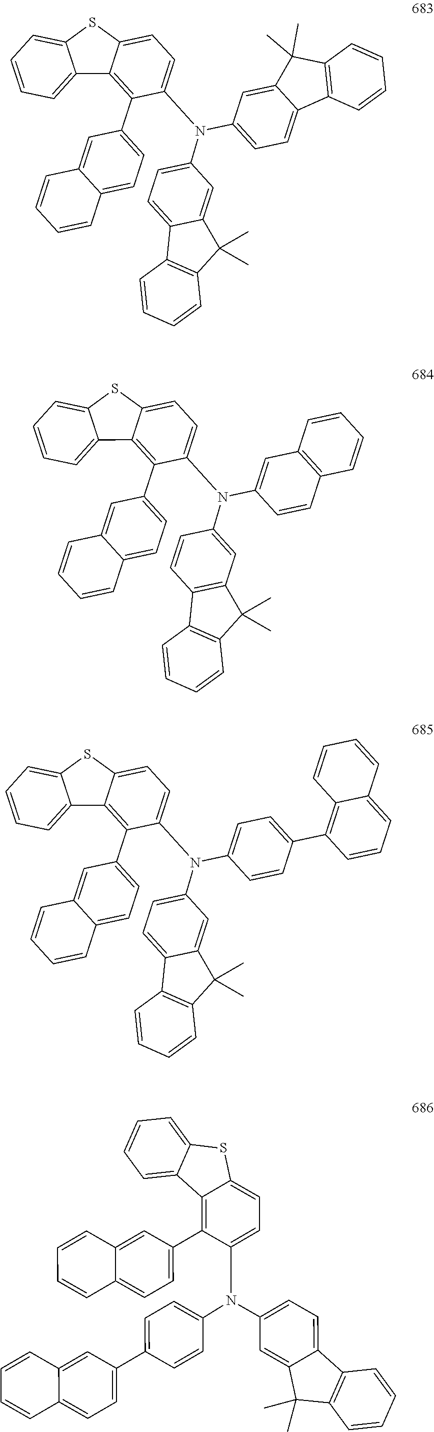 [SMILES: CC1(C)c2ccccc2-c2ccc(N(c3ccc4c(c3)C(C)(C)c3ccccc3-4)c3ccc4sc5ccccc5c4c3-c3ccc4ccccc4c3)cc21 ; CC1(C)c2ccccc2-c2ccc(N(c3ccc4ccccc4c3)c3ccc4sc5ccccc5c4c3-c3ccc4ccccc4c3)cc21 ; CC1(C)c2ccccc2-c2ccc(N(c3ccc(-c4cccc5ccccc45)cc3)c3ccc4sc5ccccc5c4c3-c3ccc4ccccc4c3)cc21 ; CC1(C)c2ccccc2-c2ccc(N(c3ccc(-c4ccc5ccccc5c4)cc3)c3ccc4sc5ccccc5c4c3-c3ccc4ccccc4c3)cc21]

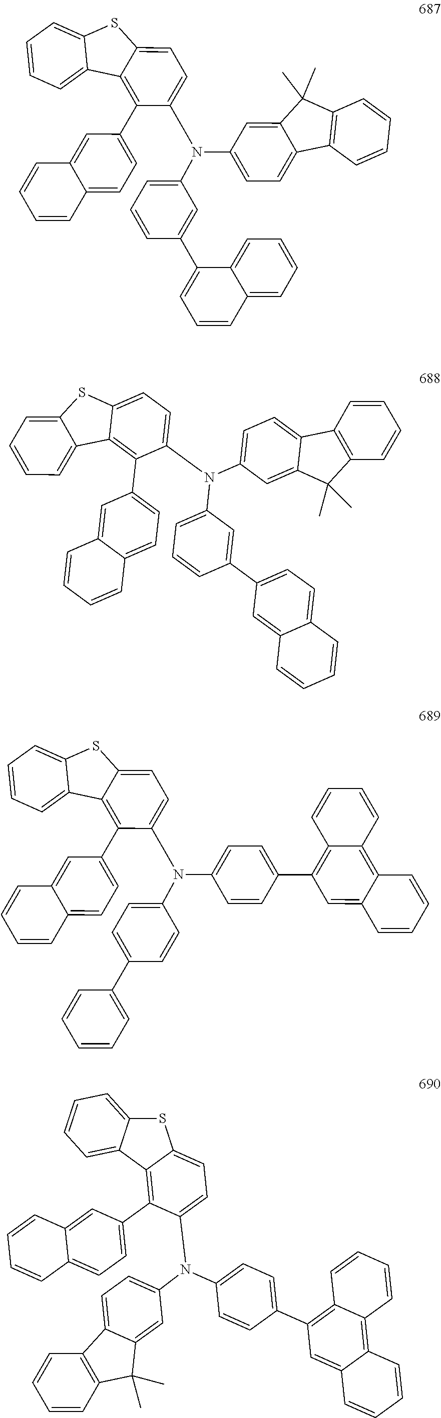

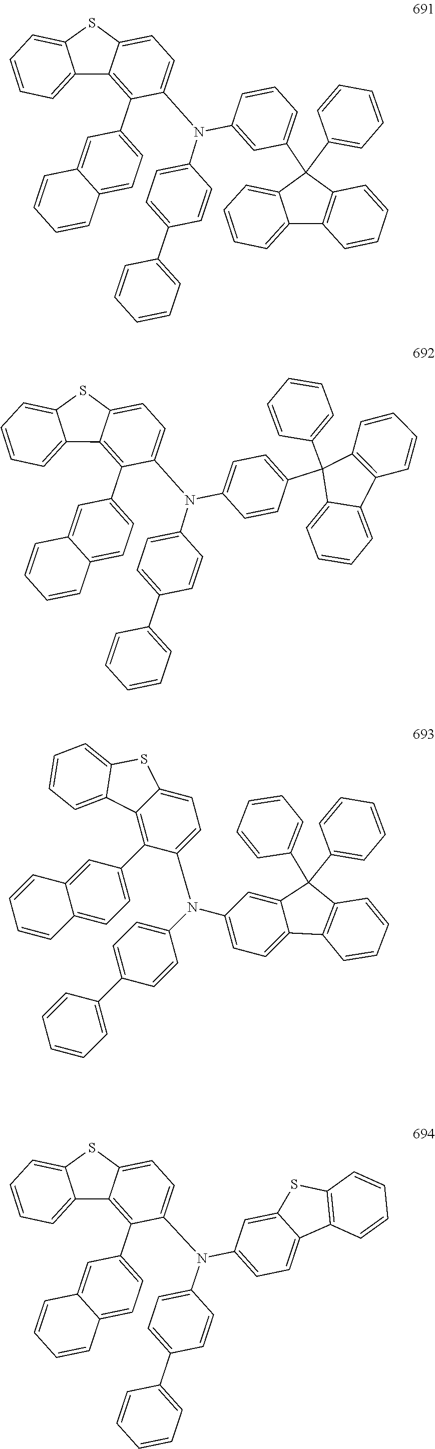 [SMILES: c1ccc(-c2ccc(N(c3cccc(C4(c5ccccc5)c5ccccc5-c5ccccc54)c3)c3ccc4sc5ccccc5c4c3-c3ccc4ccccc4c3)cc2)cc1 ; c1ccc(-c2ccc(N(c3ccc(C4(c5ccccc5)c5ccccc5-c5ccccc54)cc3)c3ccc4sc5ccccc5c4c3-c3ccc4ccccc4c3)cc2)cc1 ; c1ccc(-c2ccc(N(c3ccc4c(c3)C(c3ccccc3)(c3ccccc3)c3ccccc3-4)c3ccc4sc5ccccc5c4c3-c3ccc4ccccc4c3)cc2)cc1 ; c1ccc(-c2ccc(N(c3ccc4c(c3)sc3ccccc34)c3ccc4sc5ccccc5c4c3-c3ccc4ccccc4c3)cc2)cc1]

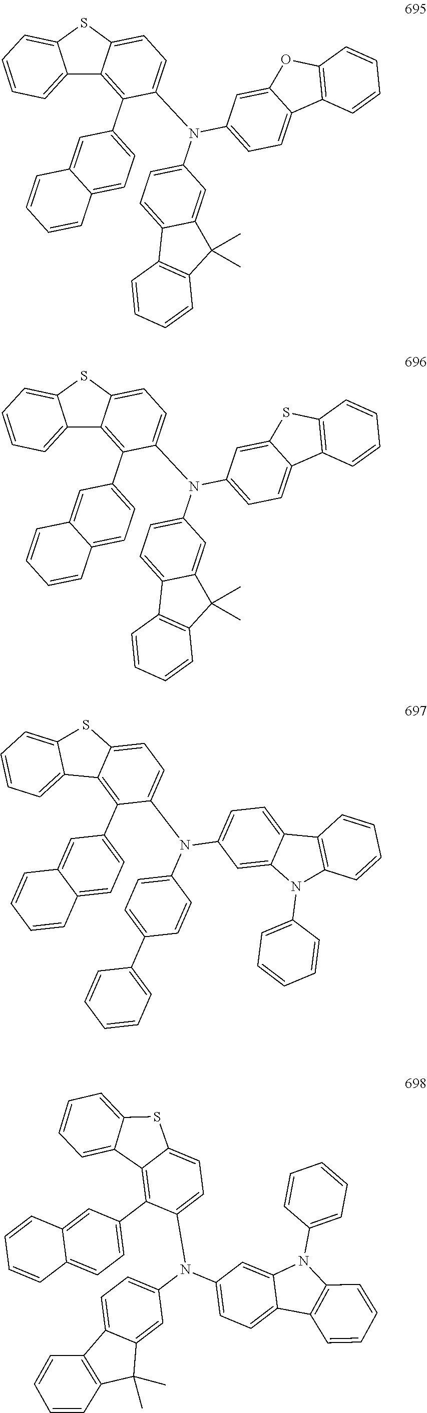 [SMILES: CC1(C)c2ccccc2-c2ccc(N(c3ccc4c(c3)oc3ccccc34)c3ccc4sc5ccccc5c4c3-c3ccc4ccccc4c3)cc21 ; CC1(C)c2ccccc2-c2ccc(N(c3ccc4c(c3)sc3ccccc34)c3ccc4sc5ccccc5c4c3-c3ccc4ccccc4c3)cc21 ; c1ccc(-c2ccc(N(c3ccc4c5ccccc5n(-c5ccccc5)c4c3)c3ccc4sc5ccccc5c4c3-c3ccc4ccccc4c3)cc2)cc1 ; CC1(C)c2ccccc2-c2ccc(N(c3ccc4c5ccccc5n(-c5ccccc5)c4c3)c3ccc4sc5ccccc5c4c3-c3ccc4ccccc4c3)cc21]

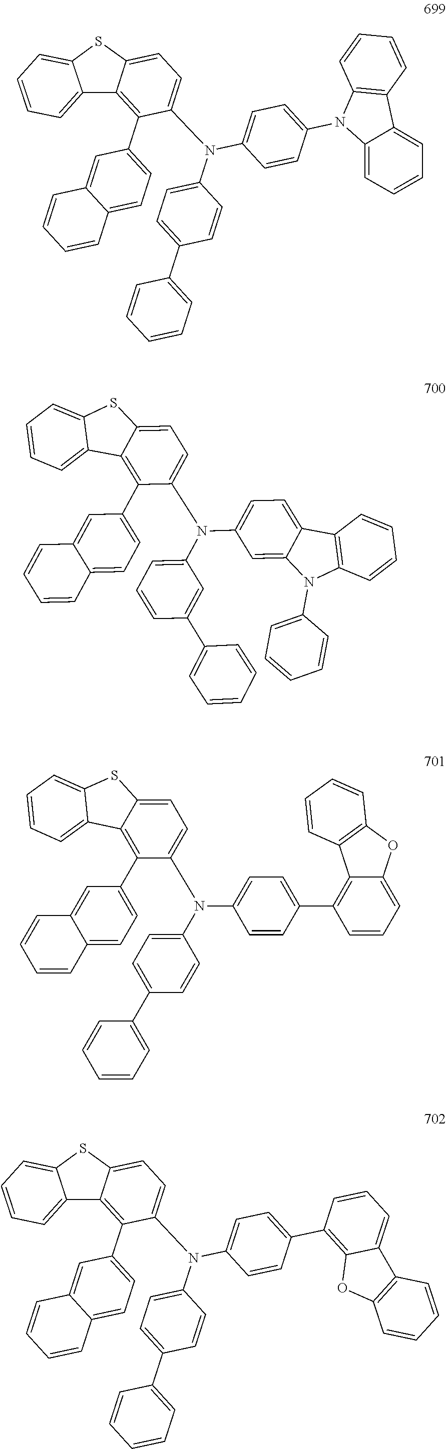 [SMILES: c1ccc(-c2ccc(N(c3ccc(-n4c5ccccc5c5ccccc54)cc3)c3ccc4sc5ccccc5c4c3-c3ccc4ccccc4c3)cc2)cc1 ; c1ccc(-c2cccc(N(c3ccc4c5ccccc5n(-c5ccccc5)c4c3)c3ccc4sc5ccccc5c4c3-c3ccc4ccccc4c3)c2)cc1 ; c1ccc(-c2ccc(N(c3ccc(-c4cccc5oc6ccccc6c45)cc3)c3ccc4sc5ccccc5c4c3-c3ccc4ccccc4c3)cc2)cc1 ; c1ccc(-c2ccc(N(c3ccc(-c4cccc5c4oc4ccccc45)cc3)c3ccc4sc5ccccc5c4c3-c3ccc4ccccc4c3)cc2)cc1]

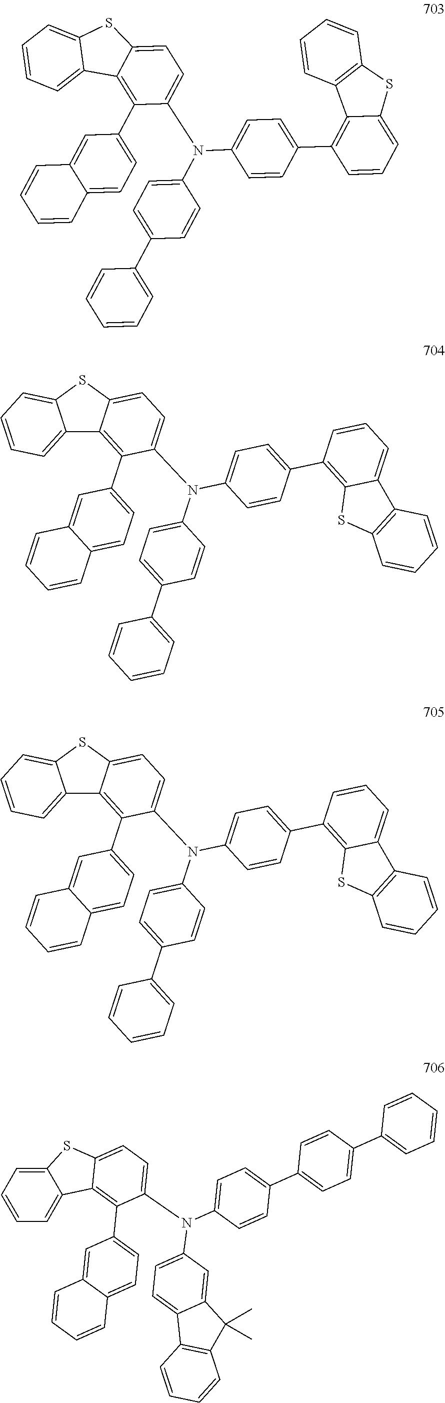 [SMILES: c1ccc(-c2ccc(N(c3ccc(-c4cccc5sc6ccccc6c45)cc3)c3ccc4sc5ccccc5c4c3-c3ccc4ccccc4c3)cc2)cc1 ; c1ccc(-c2ccc(N(c3ccc(-c4cccc5c4sc4ccccc45)cc3)c3ccc4sc5ccccc5c4c3-c3ccc4ccccc4c3)cc2)cc1 ; c1ccc(-c2ccc(N(c3ccc(-c4cccc5c4sc4ccccc45)cc3)c3ccc4sc5ccccc5c4c3-c3ccc4ccccc4c3)cc2)cc1 ; CC1(C)c2ccccc2-c2ccc(N(c3ccc(-c4ccc(-c5ccccc5)cc4)cc3)c3ccc4sc5ccccc5c4c3-c3ccc4ccccc4c3)cc21]

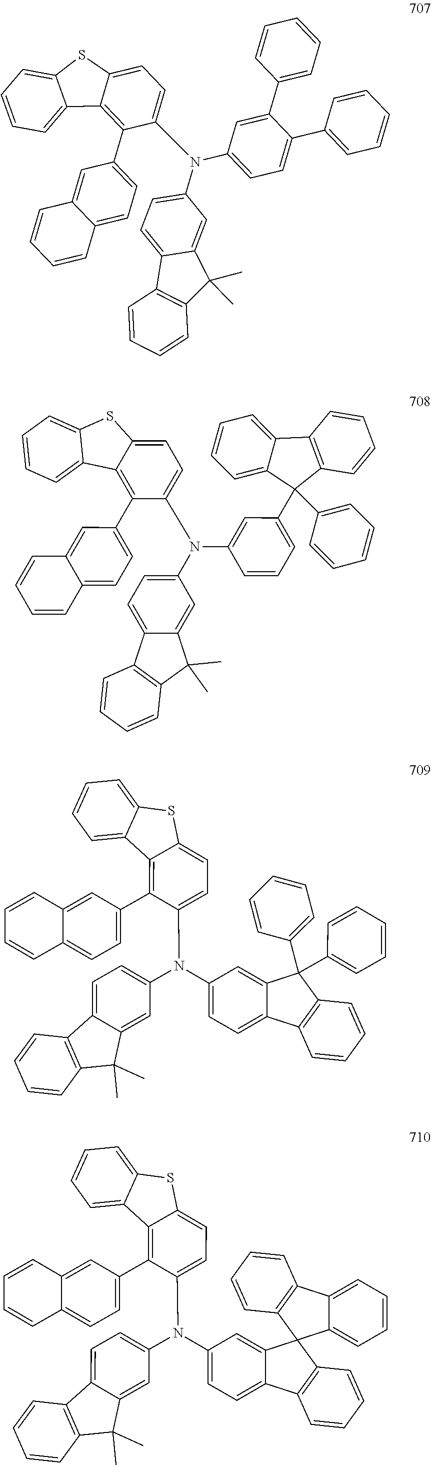 [SMILES: CC1(C)c2ccccc2-c2ccc(N(c3ccc(-c4ccccc4)c(-c4ccccc4)c3)c3ccc4sc5ccccc5c4c3-c3ccc4ccccc4c3)cc21 ; CC1(C)c2ccccc2-c2ccc(N(c3cccc(C4(c5ccccc5)c5ccccc5-c5ccccc54)c3)c3ccc4sc5ccccc5c4c3-c3ccc4ccccc4c3)cc21 ; CC1(C)c2ccccc2-c2ccc(N(c3ccc4c(c3)C(c3ccccc3)(c3ccccc3)c3ccccc3-4)c3ccc4sc5ccccc5c4c3-c3ccc4ccccc4c3)cc21 ; CC1(C)c2ccccc2-c2ccc(N(c3ccc4c(c3)C3(c5ccccc5-c5ccccc53)c3ccccc3-4)c3ccc4sc5ccccc5c4c3-c3ccc4ccccc4c3)cc21]

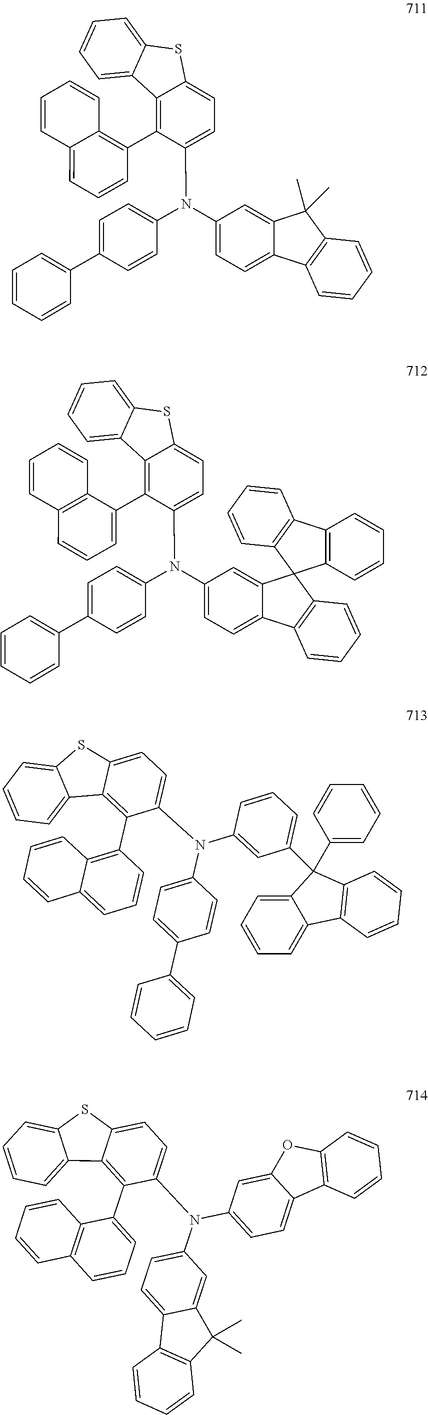 [SMILES: CC1(C)c2ccccc2-c2ccc(N(c3ccc(-c4ccccc4)cc3)c3ccc4sc5ccccc5c4c3-c3cccc4ccccc34)cc21 ; c1ccc(-c2ccc(N(c3ccc4c(c3)C3(c5ccccc5-c5ccccc53)c3ccccc3-4)c3ccc4sc5ccccc5c4c3-c3cccc4ccccc34)cc2)cc1 ; c1ccc(-c2ccc(N(c3cccc(C4(c5ccccc5)c5ccccc5-c5ccccc54)c3)c3ccc4sc5ccccc5c4c3-c3cccc4ccccc34)cc2)cc1 ; CC1(C)c2ccccc2-c2ccc(N(c3ccc4c(c3)oc3ccccc34)c3ccc4sc5ccccc5c4c3-c3cccc4ccccc34)cc21]

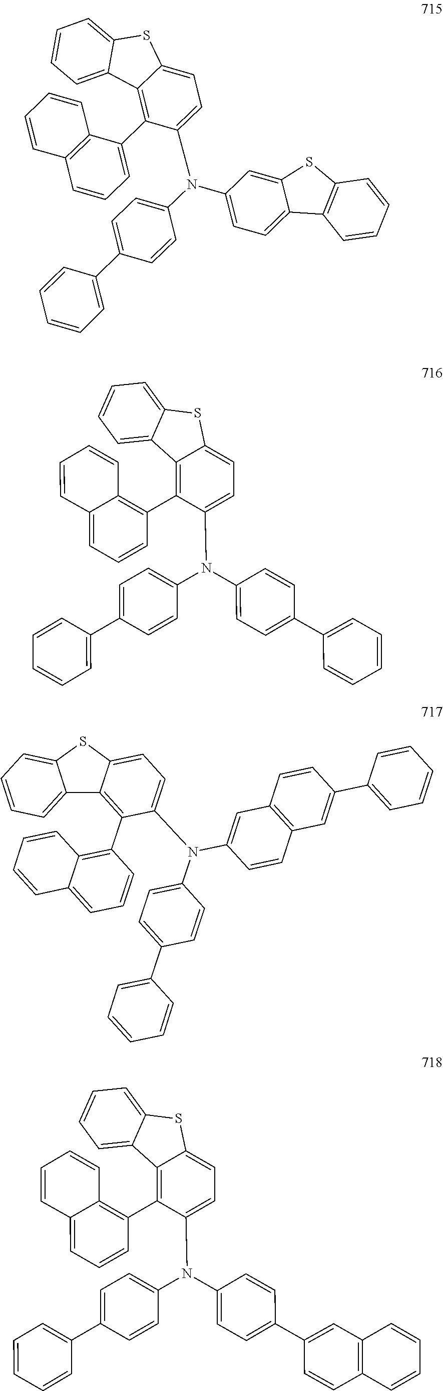

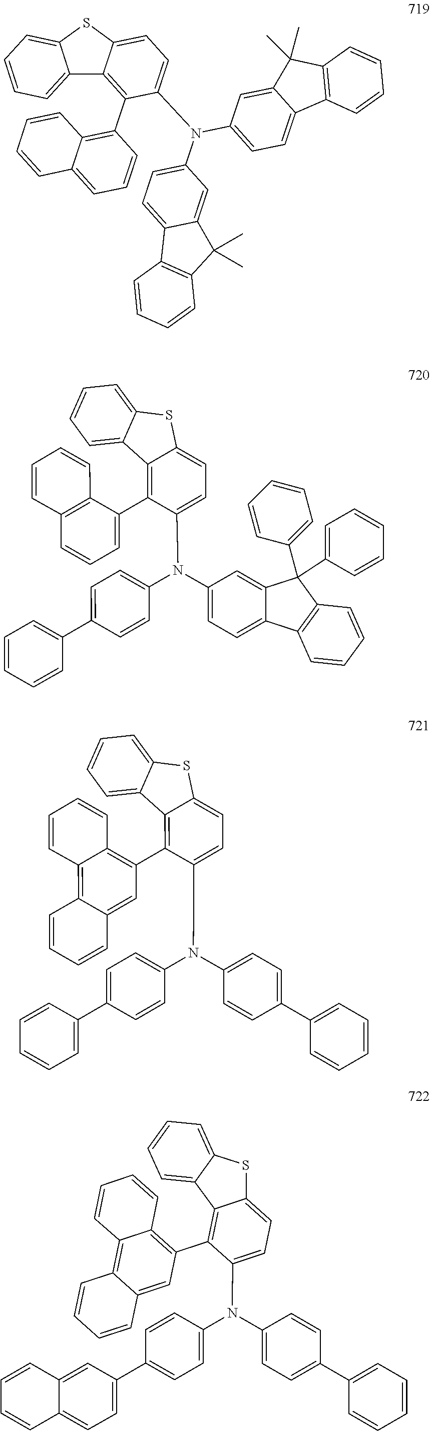 [SMILES: CC1(C)c2ccccc2-c2ccc(N(c3ccc4c(c3)C(C)(C)c3ccccc3-4)c3ccc4sc5ccccc5c4c3-c3cccc4ccccc34)cc21 ; c1ccc(-c2ccc(N(c3ccc4c(c3)C(c3ccccc3)(c3ccccc3)c3ccccc3-4)c3ccc4sc5ccccc5c4c3-c3cccc4ccccc34)cc2)cc1 ; c1ccc(-c2ccc(N(c3ccc(-c4ccccc4)cc3)c3ccc4sc5ccccc5c4c3-c3cc4ccccc4c4ccccc34)cc2)cc1 ; c1ccc(-c2ccc(N(c3ccc(-c4ccc5ccccc5c4)cc3)c3ccc4sc5ccccc5c4c3-c3cc4ccccc4c4ccccc34)cc2)cc1]

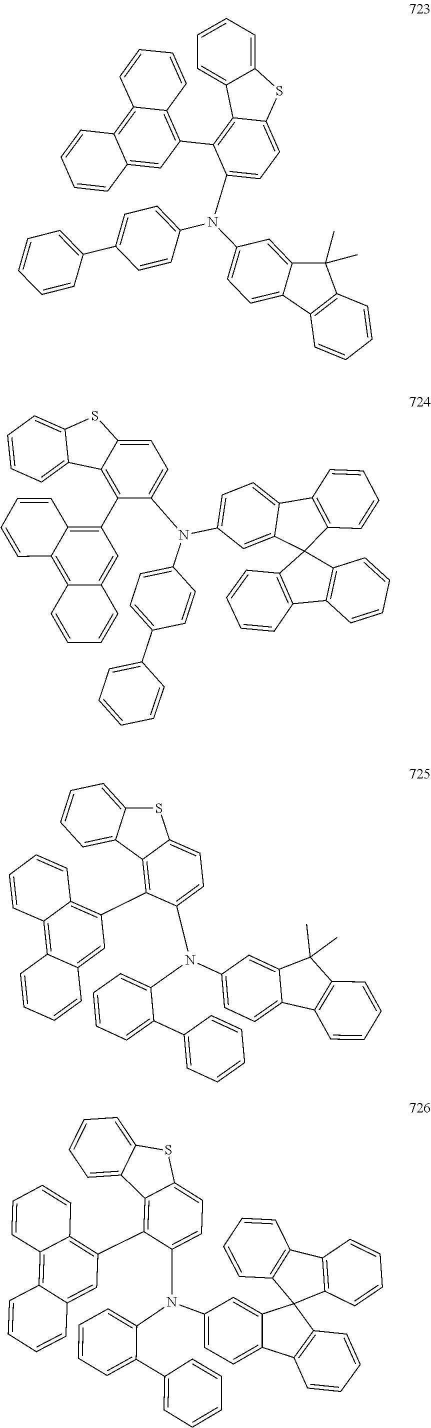 [SMILES: CC1(C)c2ccccc2-c2ccc(N(c3ccc(-c4ccccc4)cc3)c3ccc4sc5ccccc5c4c3-c3cc4ccccc4c4ccccc34)cc21 ; c1ccc(-c2ccc(N(c3ccc4c(c3)C3(c5ccccc5-c5ccccc53)c3ccccc3-4)c3ccc4sc5ccccc5c4c3-c3cc4ccccc4c4ccccc34)cc2)cc1 ; CC1(C)c2ccccc2-c2ccc(N(c3ccccc3-c3ccccc3)c3ccc4sc5ccccc5c4c3-c3cc4ccccc4c4ccccc34)cc21 ; c1ccc(-c2ccccc2N(c2ccc3c(c2)C2(c4ccccc4-c4ccccc42)c2ccccc2-3)c2ccc3sc4ccccc4c3c2-c2cc3ccccc3c3ccccc23)cc1]

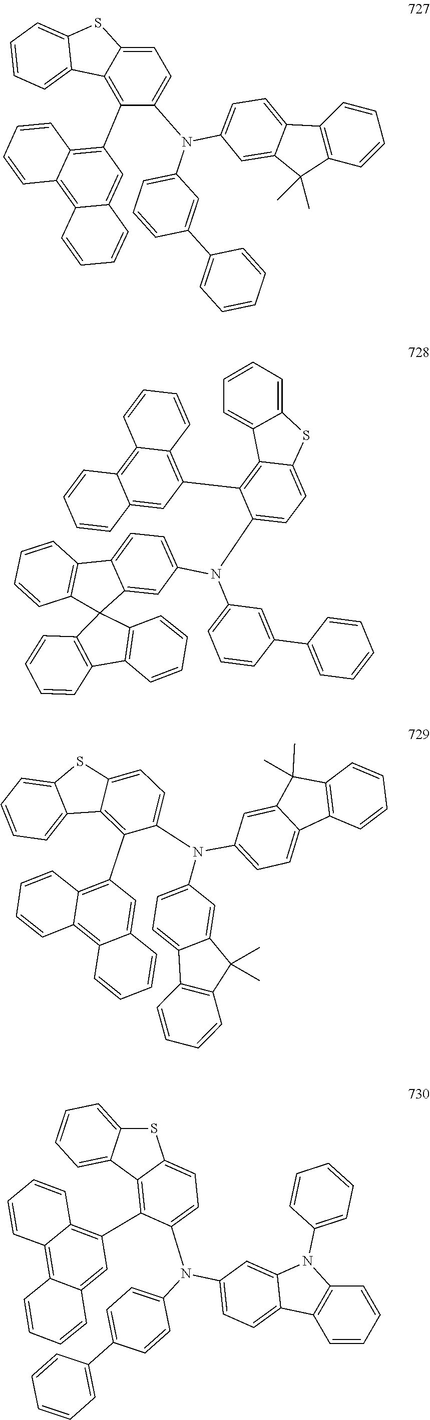 [SMILES: CC1(C)c2ccccc2-c2ccc(N(c3cccc(-c4ccccc4)c3)c3ccc4sc5ccccc5c4c3-c3cc4ccccc4c4ccccc34)cc21 ; c1ccc(-c2cccc(N(c3ccc4c(c3)C3(c5ccccc5-c5ccccc53)c3ccccc3-4)c3ccc4sc5ccccc5c4c3-c3cc4ccccc4c4ccccc34)c2)cc1 ; CC1(C)c2ccccc2-c2ccc(N(c3ccc4c(c3)C(C)(C)c3ccccc3-4)c3ccc4sc5ccccc5c4c3-c3cc4ccccc4c4ccccc34)cc21 ; c1ccc(-c2ccc(N(c3ccc4c5ccccc5n(-c5ccccc5)c4c3)c3ccc4sc5ccccc5c4c3-c3cc4ccccc4c4ccccc34)cc2)cc1]

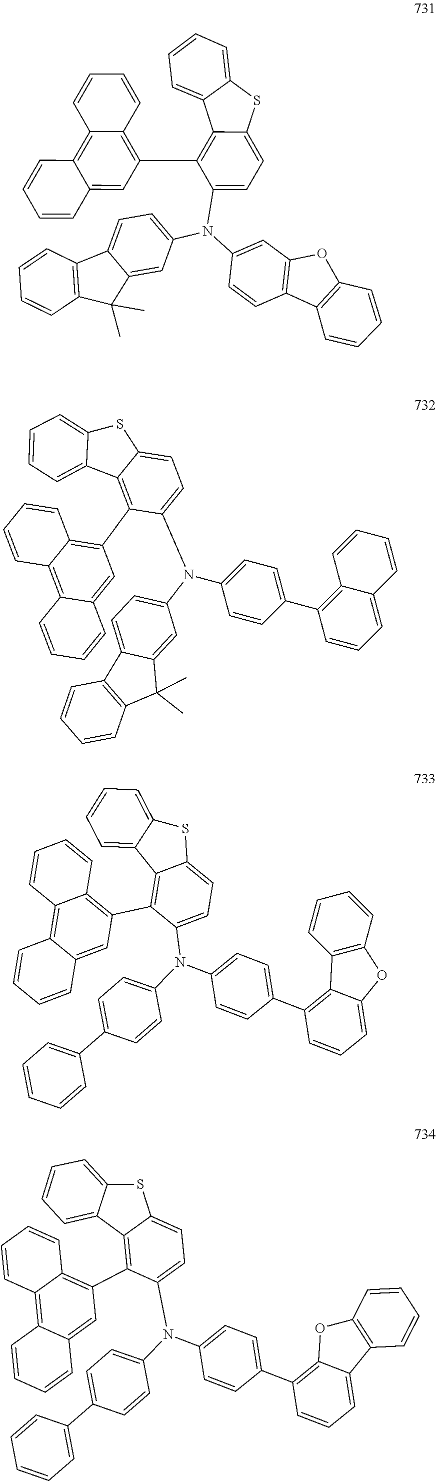 [SMILES: CC1(C)c2ccccc2-c2ccc(N(c3ccc4c(c3)oc3ccccc34)c3ccc4sc5ccccc5c4c3-c3cc4ccccc4c4ccccc34)cc21 ; CC1(C)c2ccccc2-c2ccc(N(c3ccc(-c4cccc5ccccc45)cc3)c3ccc4sc5ccccc5c4c3-c3cc4ccccc4c4ccccc34)cc21 ; c1ccc(-c2ccc(N(c3ccc(-c4cccc5oc6ccccc6c45)cc3)c3ccc4sc5ccccc5c4c3-c3cc4ccccc4c4ccccc34)cc2)cc1 ; c1ccc(-c2ccc(N(c3ccc(-c4cccc5c4oc4ccccc45)cc3)c3ccc4sc5ccccc5c4c3-c3cc4ccccc4c4ccccc34)cc2)cc1]

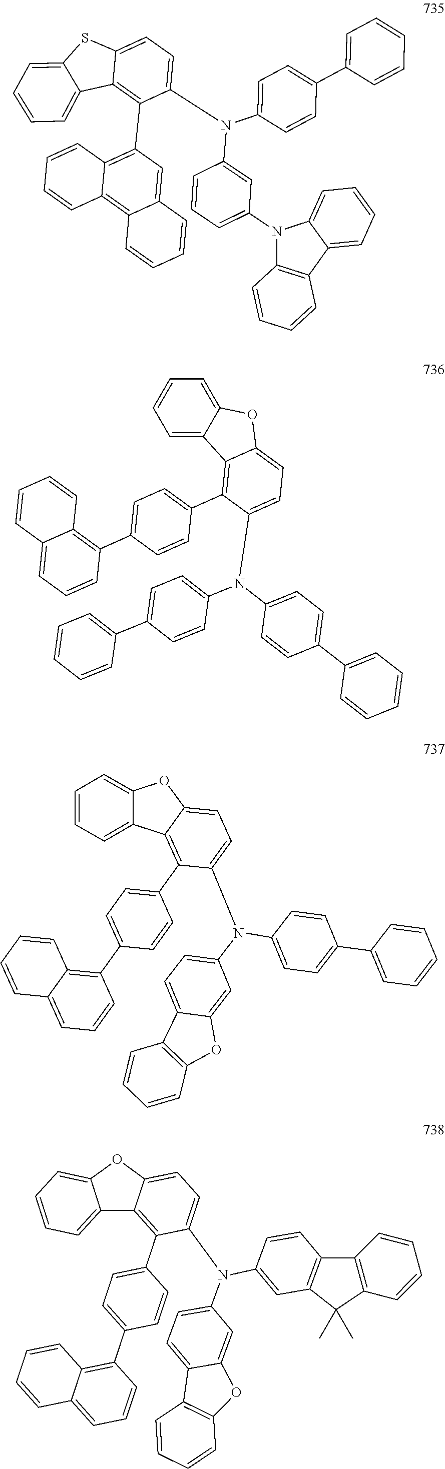 [SMILES: c1ccc(-c2ccc(N(c3cccc(-n4c5ccccc5c5ccccc54)c3)c3ccc4sc5ccccc5c4c3-c3cc4ccccc4c4ccccc34)cc2)cc1 ; c1ccc(-c2ccc(N(c3ccc(-c4ccccc4)cc3)c3ccc4oc5ccccc5c4c3-c3ccc(-c4cccc5ccccc45)cc3)cc2)cc1 ; c1ccc(-c2ccc(N(c3ccc4c(c3)oc3ccccc34)c3ccc4oc5ccccc5c4c3-c3ccc(-c4cccc5ccccc45)cc3)cc2)cc1 ; CC1(C)c2ccccc2-c2ccc(N(c3ccc4c(c3)oc3ccccc34)c3ccc4oc5ccccc5c4c3-c3ccc(-c4cccc5ccccc45)cc3)cc21]

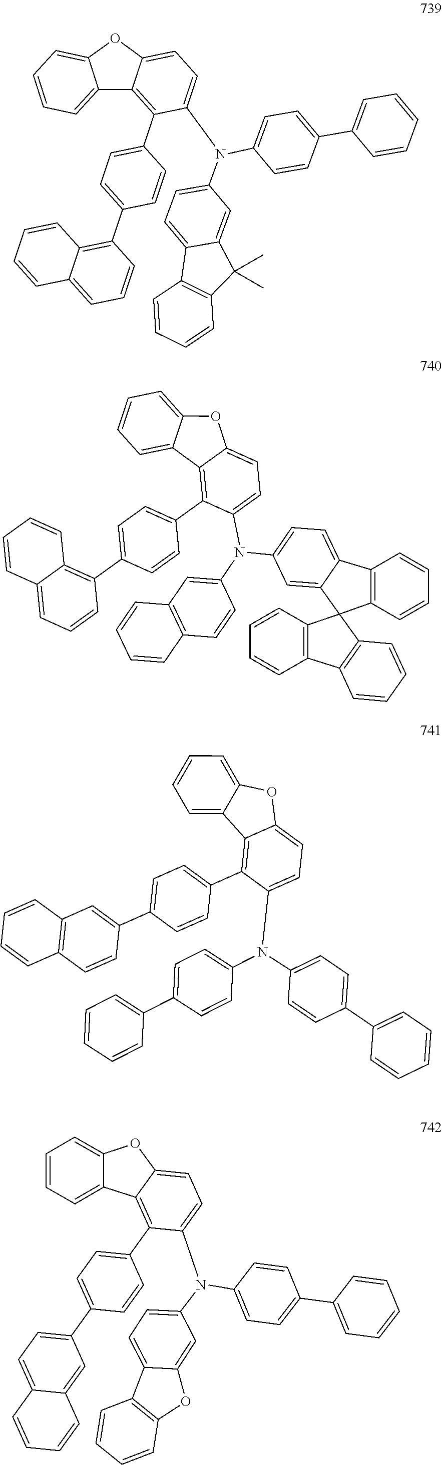 [SMILES: CC1(C)c2ccccc2-c2ccc(N(c3ccc(-c4ccccc4)cc3)c3ccc4oc5ccccc5c4c3-c3ccc(-c4cccc5ccccc45)cc3)cc21 ; c1ccc2c(c1)-c1ccccc1C21c2ccccc2-c2ccc(N(c3ccc4ccccc4c3)c3ccc4oc5ccccc5c4c3-c3ccc(-c4cccc5ccccc45)cc3)cc21 ; c1ccc(-c2ccc(N(c3ccc(-c4ccccc4)cc3)c3ccc4oc5ccccc5c4c3-c3ccc(-c4ccc5ccccc5c4)cc3)cc2)cc1 ; c1ccc(-c2ccc(N(c3ccc4c(c3)oc3ccccc34)c3ccc4oc5ccccc5c4c3-c3ccc(-c4ccc5ccccc5c4)cc3)cc2)cc1]

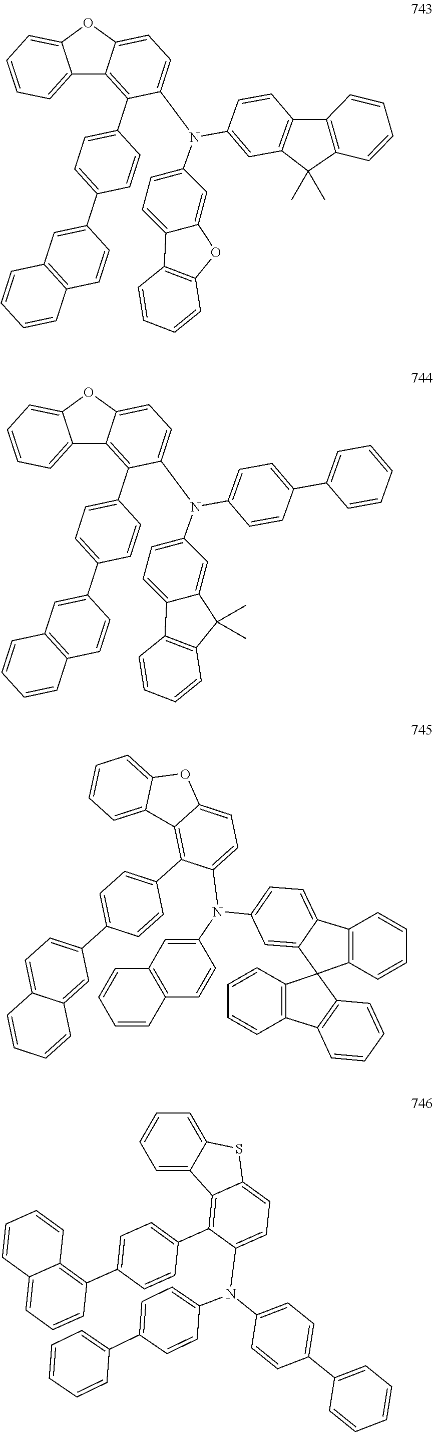 [SMILES: CC1(C)c2ccccc2-c2ccc(N(c3ccc4c(c3)oc3ccccc34)c3ccc4oc5ccccc5c4c3-c3ccc(-c4ccc5ccccc5c4)cc3)cc21 ; CC1(C)c2ccccc2-c2ccc(N(c3ccc(-c4ccccc4)cc3)c3ccc4oc5ccccc5c4c3-c3ccc(-c4ccc5ccccc5c4)cc3)cc21 ; c1ccc2c(c1)-c1ccccc1C21c2ccccc2-c2ccc(N(c3ccc4ccccc4c3)c3ccc4oc5ccccc5c4c3-c3ccc(-c4ccc5ccccc5c4)cc3)cc21 ; c1ccc(-c2ccc(N(c3ccc(-c4ccccc4)cc3)c3ccc4sc5ccccc5c4c3-c3ccc(-c4cccc5ccccc45)cc3)cc2)cc1]

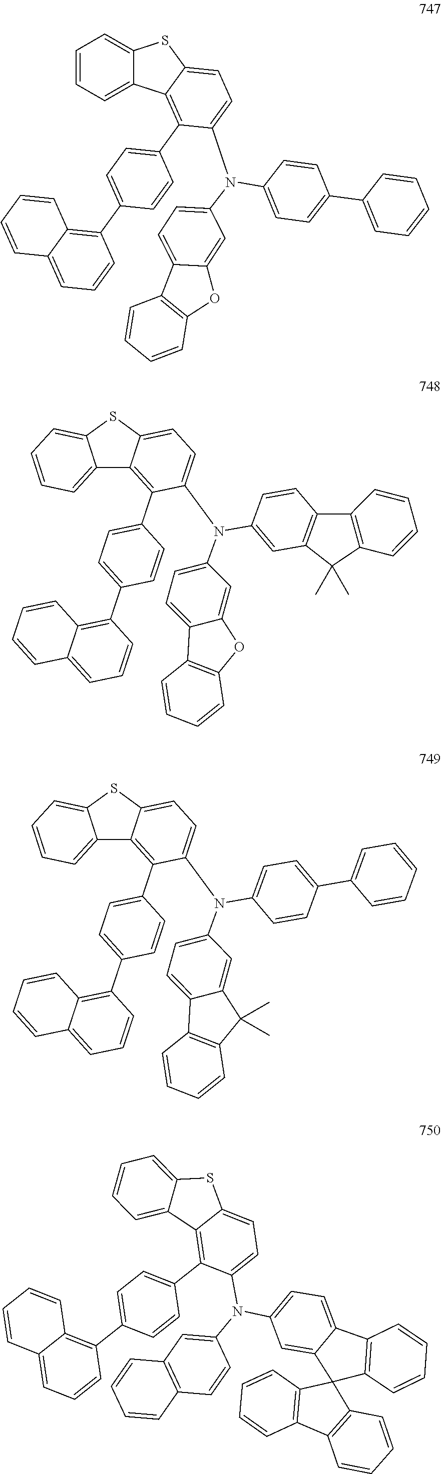 [SMILES: c1ccc(-c2ccc(N(c3ccc4c(c3)oc3ccccc34)c3ccc4sc5ccccc5c4c3-c3ccc(-c4cccc5ccccc45)cc3)cc2)cc1 ; CC1(C)c2ccccc2-c2ccc(N(c3ccc4c(c3)oc3ccccc34)c3ccc4sc5ccccc5c4c3-c3ccc(-c4cccc5ccccc45)cc3)cc21 ; CC1(C)c2ccccc2-c2ccc(N(c3ccc(-c4ccccc4)cc3)c3ccc4sc5ccccc5c4c3-c3ccc(-c4cccc5ccccc45)cc3)cc21 ; c1ccc2c(c1)-c1ccccc1C21c2ccccc2-c2ccc(N(c3ccc4ccccc4c3)c3ccc4sc5ccccc5c4c3-c3ccc(-c4cccc5ccccc45)cc3)cc21]

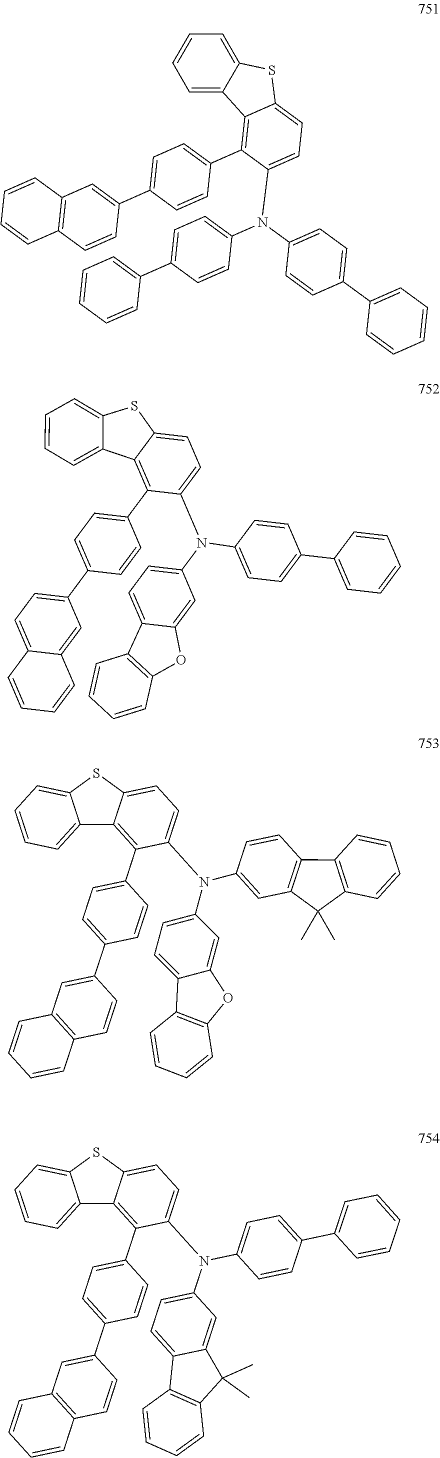 [SMILES: c1ccc(-c2ccc(N(c3ccc(-c4ccccc4)cc3)c3ccc4sc5ccccc5c4c3-c3ccc(-c4ccc5ccccc5c4)cc3)cc2)cc1 ; c1ccc(-c2ccc(N(c3ccc4c(c3)oc3ccccc34)c3ccc4sc5ccccc5c4c3-c3ccc(-c4ccc5ccccc5c4)cc3)cc2)cc1 ; CC1(C)c2ccccc2-c2ccc(N(c3ccc4c(c3)oc3ccccc34)c3ccc4sc5ccccc5c4c3-c3ccc(-c4ccc5ccccc5c4)cc3)cc21 ; CC1(C)c2ccccc2-c2ccc(N(c3ccc(-c4ccccc4)cc3)c3ccc4sc5ccccc5c4c3-c3ccc(-c4ccc5ccccc5c4)cc3)cc21]

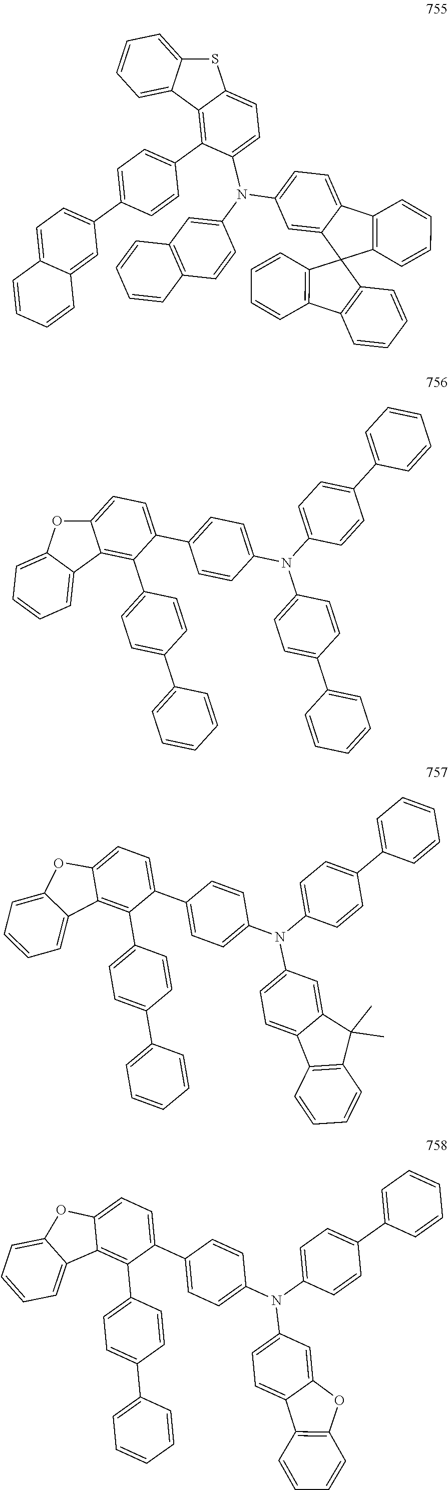 [SMILES: c1ccc2c(c1)-c1ccccc1C21c2ccccc2-c2ccc(N(c3ccc4ccccc4c3)c3ccc4sc5ccccc5c4c3-c3ccc(-c4ccc5ccccc5c4)cc3)cc21 ; c1ccc(-c2ccc(-c3c(-c4ccc(N(c5ccc(-c6ccccc6)cc5)c5ccc(-c6ccccc6)cc5)cc4)ccc4oc5ccccc5c34)cc2)cc1 ; CC1(C)c2ccccc2-c2ccc(N(c3ccc(-c4ccccc4)cc3)c3ccc(-c4ccc5oc6ccccc6c5c4-c4ccc(-c5ccccc5)cc4)cc3)cc21 ; c1ccc(-c2ccc(-c3c(-c4ccc(N(c5ccc(-c6ccccc6)cc5)c5ccc6c(c5)oc5ccccc56)cc4)ccc4oc5ccccc5c34)cc2)cc1]

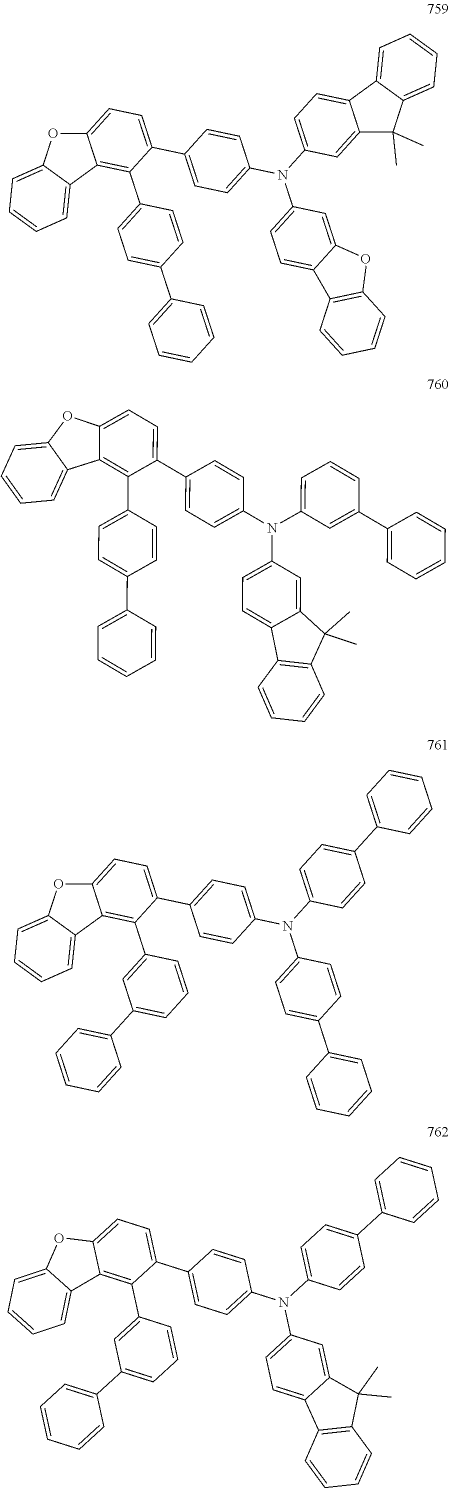 [SMILES: CC1(C)c2ccccc2-c2ccc(N(c3ccc(-c4ccc5oc6ccccc6c5c4-c4ccc(-c5ccccc5)cc4)cc3)c3ccc4c(c3)oc3ccccc34)cc21 ; CC1(C)c2ccccc2-c2ccc(N(c3ccc(-c4ccc5oc6ccccc6c5c4-c4ccc(-c5ccccc5)cc4)cc3)c3cccc(-c4ccccc4)c3)cc21 ; c1ccc(-c2ccc(N(c3ccc(-c4ccccc4)cc3)c3ccc(-c4ccc5oc6ccccc6c5c4-c4cccc(-c5ccccc5)c4)cc3)cc2)cc1 ; CC1(C)c2ccccc2-c2ccc(N(c3ccc(-c4ccccc4)cc3)c3ccc(-c4ccc5oc6ccccc6c5c4-c4cccc(-c5ccccc5)c4)cc3)cc21]

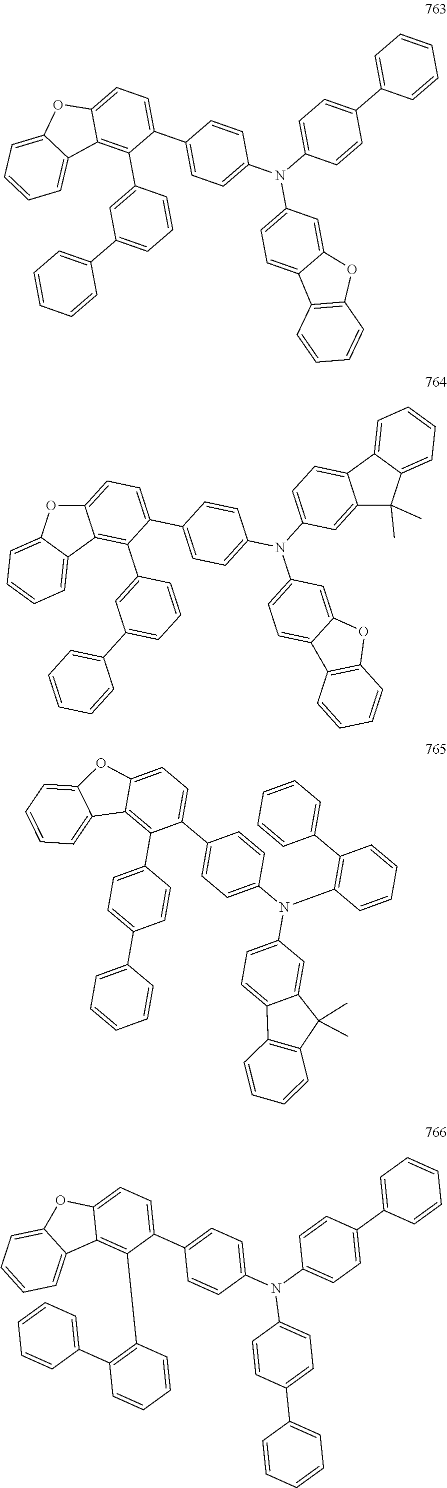 [SMILES: c1ccc(-c2ccc(N(c3ccc(-c4ccc5oc6ccccc6c5c4-c4cccc(-c5ccccc5)c4)cc3)c3ccc4c(c3)oc3ccccc34)cc2)cc1 ; CC1(C)c2ccccc2-c2ccc(N(c3ccc(-c4ccc5oc6ccccc6c5c4-c4cccc(-c5ccccc5)c4)cc3)c3ccc4c(c3)oc3ccccc34)cc21 ; CC1(C)c2ccccc2-c2ccc(N(c3ccc(-c4ccc5oc6ccccc6c5c4-c4ccc(-c5ccccc5)cc4)cc3)c3ccccc3-c3ccccc3)cc21 ; c1ccc(-c2ccc(N(c3ccc(-c4ccccc4)cc3)c3ccc(-c4ccc5oc6ccccc6c5c4-c4ccccc4-c4ccccc4)cc3)cc2)cc1]

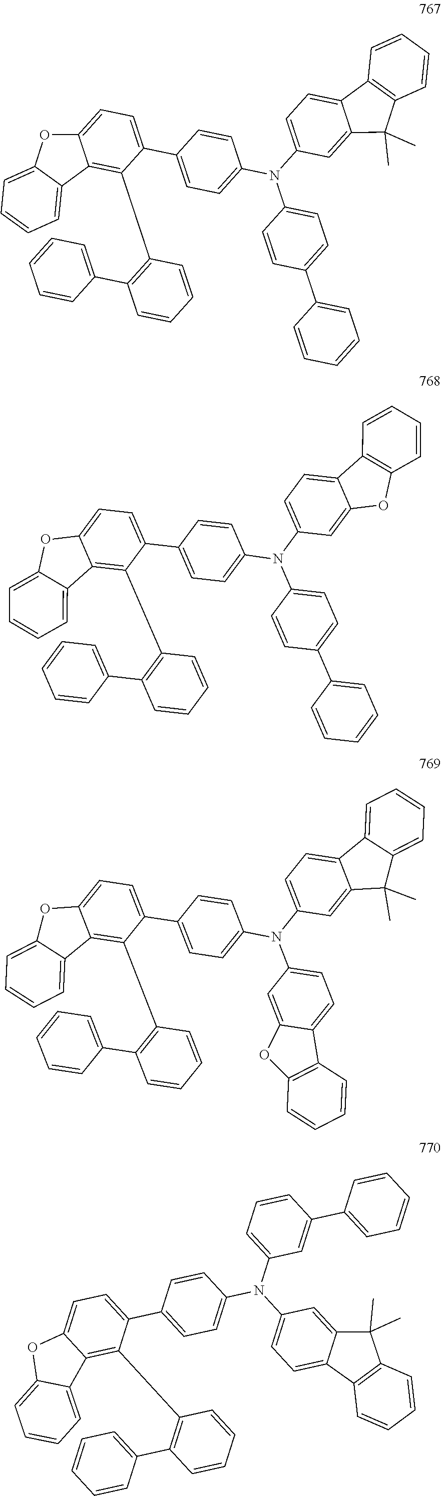 [SMILES: CC1(C)c2ccccc2-c2ccc(N(c3ccc(-c4ccccc4)cc3)c3ccc(-c4ccc5oc6ccccc6c5c4-c4ccccc4-c4ccccc4)cc3)cc21 ; c1ccc(-c2ccc(N(c3ccc(-c4ccc5oc6ccccc6c5c4-c4ccccc4-c4ccccc4)cc3)c3ccc4c(c3)oc3ccccc34)cc2)cc1 ; CC1(C)c2ccccc2-c2ccc(N(c3ccc(-c4ccc5oc6ccccc6c5c4-c4ccccc4-c4ccccc4)cc3)c3ccc4c(c3)oc3ccccc34)cc21 ; CC1(C)c2ccccc2-c2ccc(N(c3ccc(-c4ccc5oc6ccccc6c5c4-c4ccccc4-c4ccccc4)cc3)c3cccc(-c4ccccc4)c3)cc21]

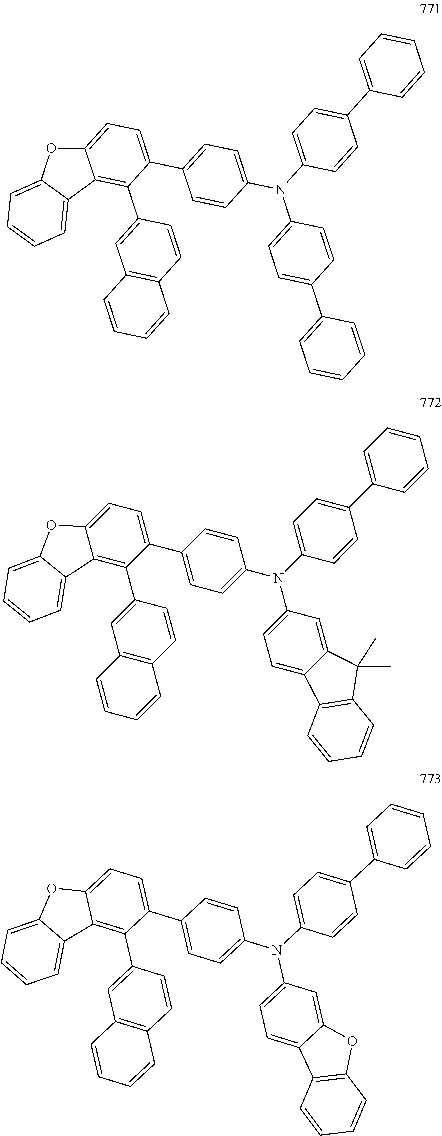 [SMILES: c1ccc(-c2ccc(N(c3ccc(-c4ccccc4)cc3)c3ccc(-c4ccc5oc6ccccc6c5c4-c4ccc5ccccc5c4)cc3)cc2)cc1 ; CC1(C)c2ccccc2-c2ccc(N(c3ccc(-c4ccccc4)cc3)c3ccc(-c4ccc5oc6ccccc6c5c4-c4ccc5ccccc5c4)cc3)cc21 ; c1ccc(-c2ccc(N(c3ccc(-c4ccc5oc6ccccc6c5c4-c4ccc5ccccc5c4)cc3)c3ccc4c(c3)oc3ccccc34)cc2)cc1]

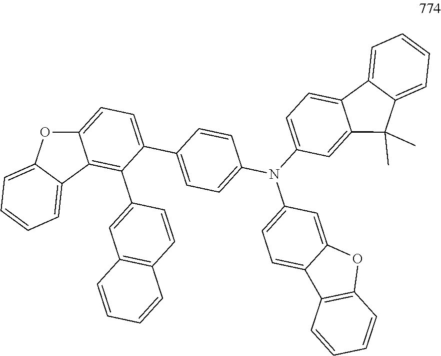 [SMILES: CC1(C)c2ccccc2-c2ccc(N(c3ccc(-c4ccc5oc6ccccc6c5c4-c4ccc5ccccc5c4)cc3)c3ccc4c(c3)oc3ccccc34)cc21]

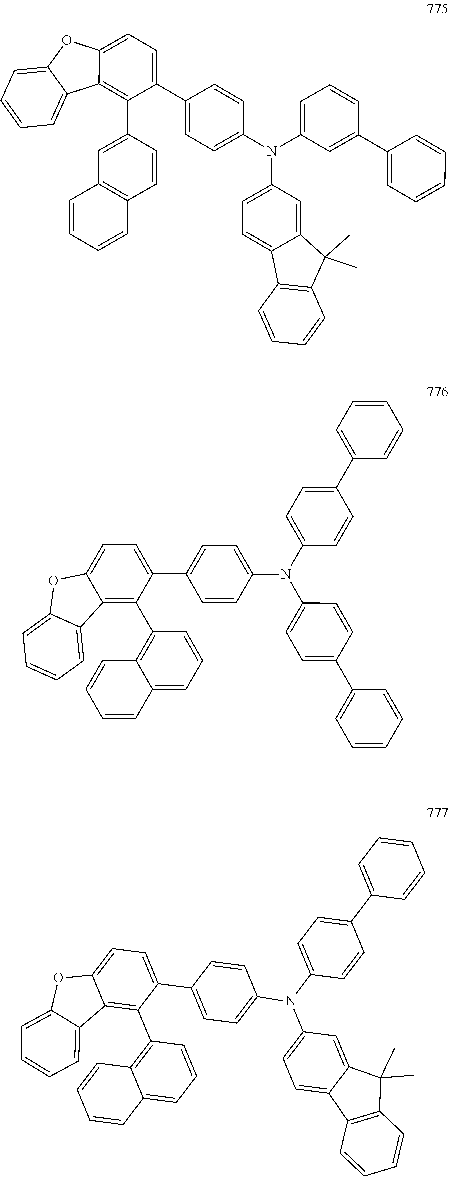 [SMILES: CC1(C)c2ccccc2-c2ccc(N(c3ccc(-c4ccc5oc6ccccc6c5c4-c4ccc5ccccc5c4)cc3)c3cccc(-c4ccccc4)c3)cc21 ; c1ccc(-c2ccc(N(c3ccc(-c4ccccc4)cc3)c3ccc(-c4ccc5oc6ccccc6c5c4-c4cccc5ccccc45)cc3)cc2)cc1 ; CC1(C)c2ccccc2-c2ccc(N(c3ccc(-c4ccccc4)cc3)c3ccc(-c4ccc5oc6ccccc6c5c4-c4cccc5ccccc45)cc3)cc21]

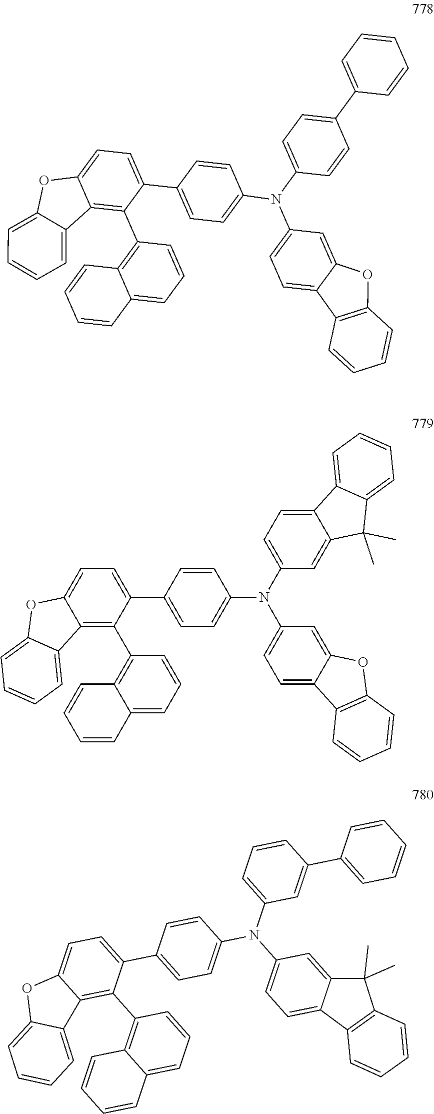 [SMILES: c1ccc(-c2ccc(N(c3ccc(-c4ccc5oc6ccccc6c5c4-c4cccc5ccccc45)cc3)c3ccc4c(c3)oc3ccccc34)cc2)cc1 ; CC1(C)c2ccccc2-c2ccc(N(c3ccc(-c4ccc5oc6ccccc6c5c4-c4cccc5ccccc45)cc3)c3ccc4c(c3)oc3ccccc34)cc21 ; CC1(C)c2ccccc2-c2ccc(N(c3ccc(-c4ccc5oc6ccccc6c5c4-c4cccc5ccccc45)cc3)c3cccc(-c4ccccc4)c3)cc21]

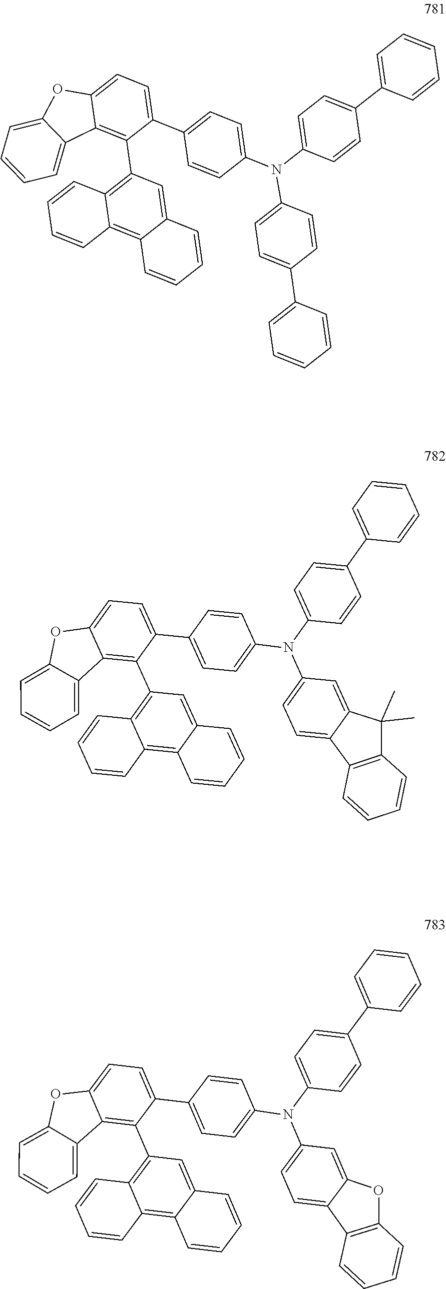 [SMILES: c1ccc(-c2ccc(N(c3ccc(-c4ccccc4)cc3)c3ccc(-c4ccc5oc6ccccc6c5c4-c4cc5ccccc5c5ccccc45)cc3)cc2)cc1 ; CC1(C)c2ccccc2-c2ccc(N(c3ccc(-c4ccccc4)cc3)c3ccc(-c4ccc5oc6ccccc6c5c4-c4cc5ccccc5c5ccccc45)cc3)cc21 ; c1ccc(-c2ccc(N(c3ccc(-c4ccc5oc6ccccc6c5c4-c4cc5ccccc5c5ccccc45)cc3)c3ccc4c(c3)oc3ccccc34)cc2)cc1]

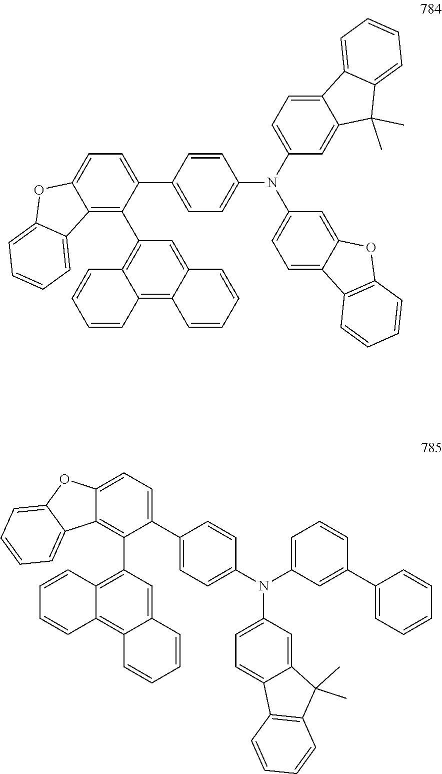 [SMILES: CC1(C)c2ccccc2-c2ccc(N(c3ccc(-c4ccc5oc6ccccc6c5c4-c4cc5ccccc5c5ccccc45)cc3)c3ccc4c(c3)oc3ccccc34)cc21 ; CC1(C)c2ccccc2-c2ccc(N(c3ccc(-c4ccc5oc6ccccc6c5c4-c4cc5ccccc5c5ccccc45)cc3)c3cccc(-c4ccccc4)c3)cc21]

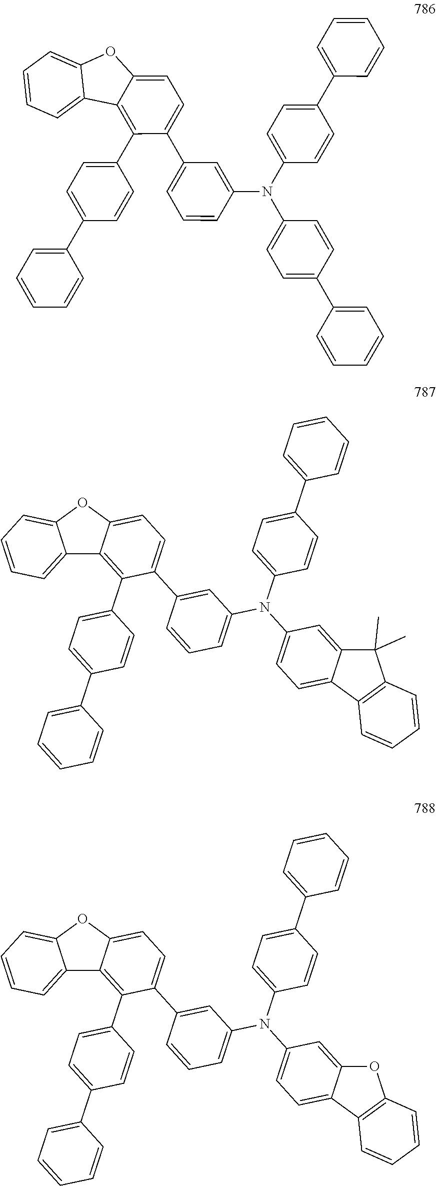 [SMILES: c1ccc(-c2ccc(-c3c(-c4cccc(N(c5ccc(-c6ccccc6)cc5)c5ccc(-c6ccccc6)cc5)c4)ccc4oc5ccccc5c34)cc2)cc1 ; CC1(C)c2ccccc2-c2ccc(N(c3ccc(-c4ccccc4)cc3)c3cccc(-c4ccc5oc6ccccc6c5c4-c4ccc(-c5ccccc5)cc4)c3)cc21 ; c1ccc(-c2ccc(-c3c(-c4cccc(N(c5ccc(-c6ccccc6)cc5)c5ccc6c(c5)oc5ccccc56)c4)ccc4oc5ccccc5c34)cc2)cc1]

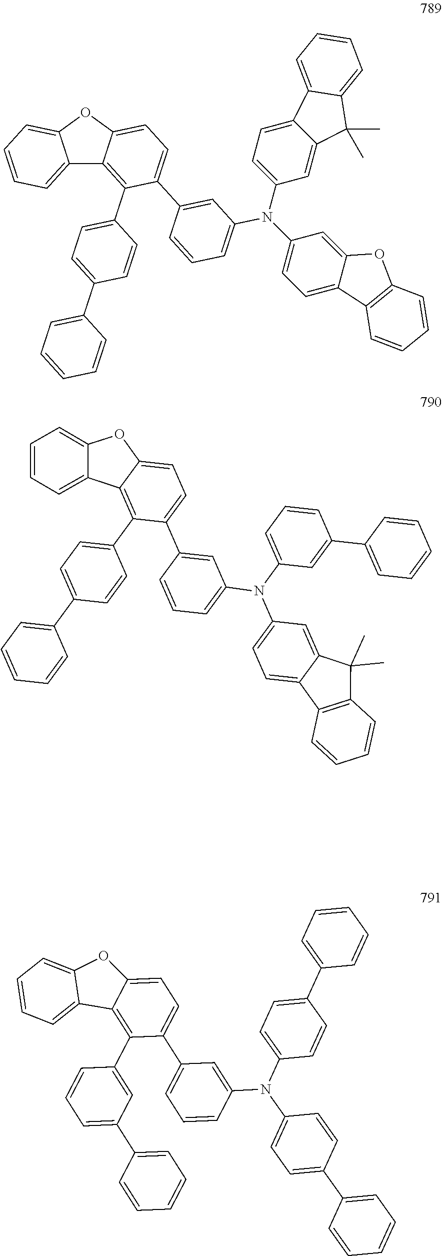 [SMILES: CC1(C)c2ccccc2-c2ccc(N(c3cccc(-c4ccc5oc6ccccc6c5c4-c4ccc(-c5ccccc5)cc4)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4ccc(-c5ccccc5)cc4)c3)cc21 ; c1ccc(-c2ccc(N(c3ccc(-c4ccccc4)cc3)c3cccc(-c4ccc5oc6ccccc6c5c4-c4cccc(-c5ccccc5)c4)c3)cc2)cc1]

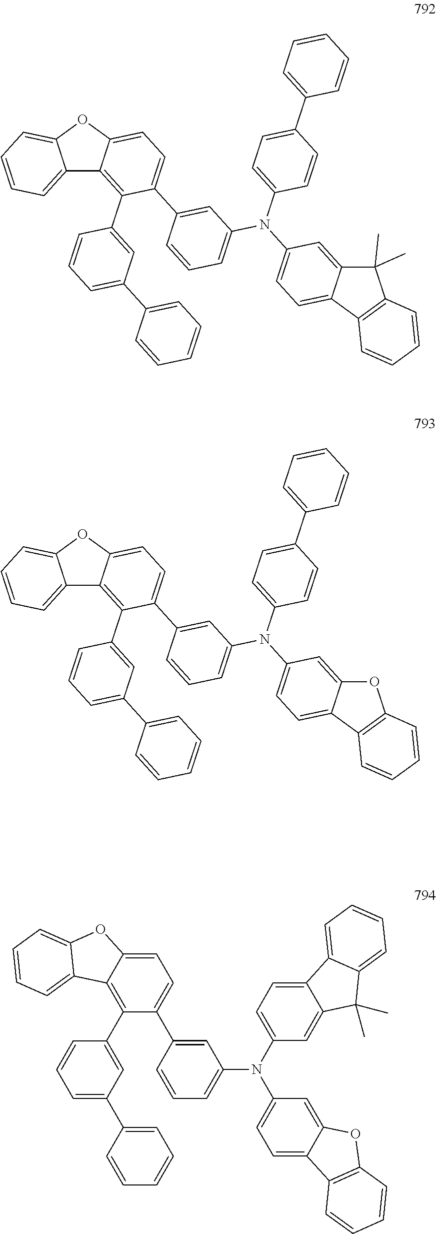 [SMILES: CC1(C)c2ccccc2-c2ccc(N(c3ccc(-c4ccccc4)cc3)c3cccc(-c4ccc5oc6ccccc6c5c4-c4cccc(-c5ccccc5)c4)c3)cc21 ; c1ccc(-c2ccc(N(c3cccc(-c4ccc5oc6ccccc6c5c4-c4cccc(-c5ccccc5)c4)c3)c3ccc4c(c3)oc3ccccc34)cc2)cc1 ; CC1(C)c2ccccc2-c2ccc(N(c3cccc(-c4ccc5oc6ccccc6c5c4-c4cccc(-c5ccccc5)c4)c3)c3ccc4c(c3)oc3ccccc34)cc21]

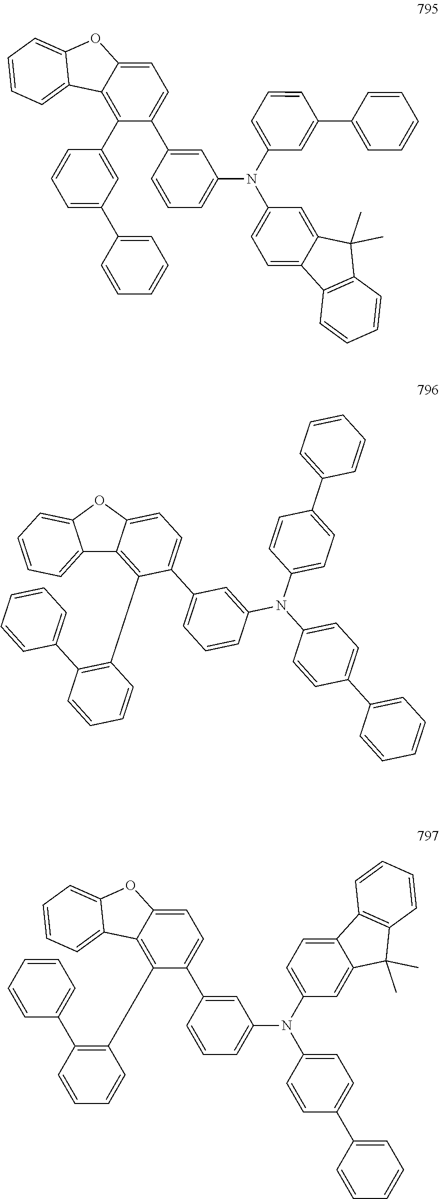 [SMILES: CC1(C)c2ccccc2-c2ccc(N(c3cccc(-c4ccccc4)c3)c3cccc(-c4ccc5oc6ccccc6c5c4-c4cccc(-c5ccccc5)c4)c3)cc21 ; c1ccc(-c2ccc(N(c3ccc(-c4ccccc4)cc3)c3cccc(-c4ccc5oc6ccccc6c5c4-c4ccccc4-c4ccccc4)c3)cc2)cc1 ; CC1(C)c2ccccc2-c2ccc(N(c3ccc(-c4ccccc4)cc3)c3cccc(-c4ccc5oc6ccccc6c5c4-c4ccccc4-c4ccccc4)c3)cc21]

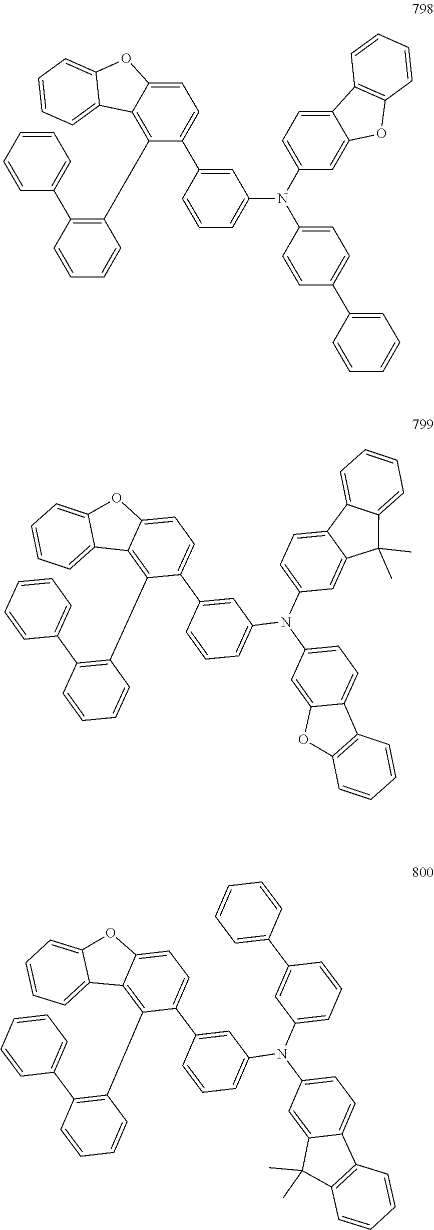 [SMILES: c1ccc(-c2ccc(N(c3cccc(-c4ccc5oc6ccccc6c5c4-c4ccccc4-c4ccccc4)c3)c3ccc4c(c3)oc3ccccc34)cc2)cc1 ; CC1(C)c2ccccc2-c2ccc(N(c3cccc(-c4ccc5oc6ccccc6c5c4-c4ccccc4-c4ccccc4)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4ccccc4-c4ccccc4)c3)cc21]

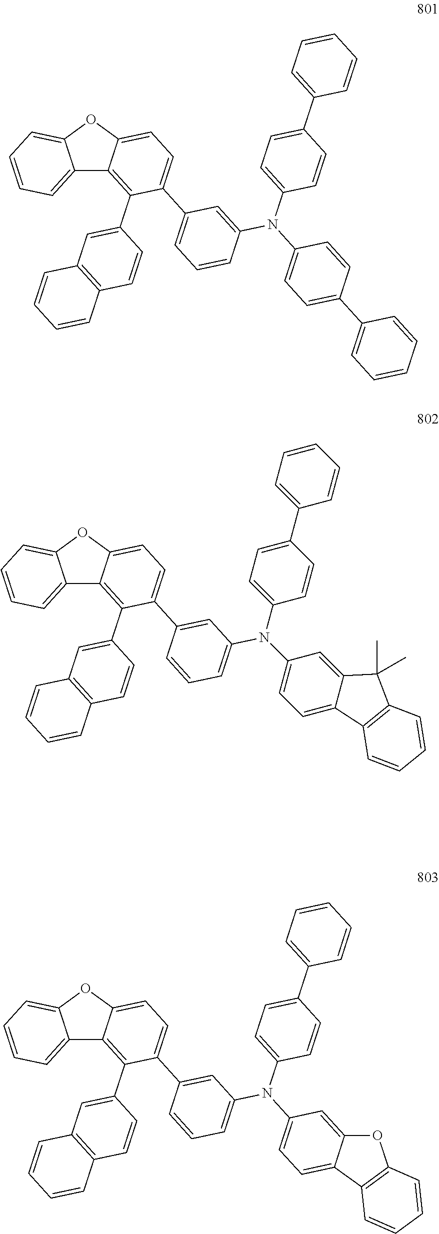 [SMILES: c1ccc(-c2ccc(N(c3ccc(-c4ccccc4)cc3)c3cccc(-c4ccc5oc6ccccc6c5c4-c4ccc5ccccc5c4)c3)cc2)cc1 ; CC1(C)c2ccccc2-c2ccc(N(c3ccc(-c4ccccc4)cc3)c3cccc(-c4ccc5oc6ccccc6c5c4-c4ccc5ccccc5c4)c3)cc21 ; c1ccc(-c2ccc(N(c3cccc(-c4ccc5oc6ccccc6c5c4-c4ccc5ccccc5c4)c3)c3ccc4c(c3)oc3ccccc34)cc2)cc1]

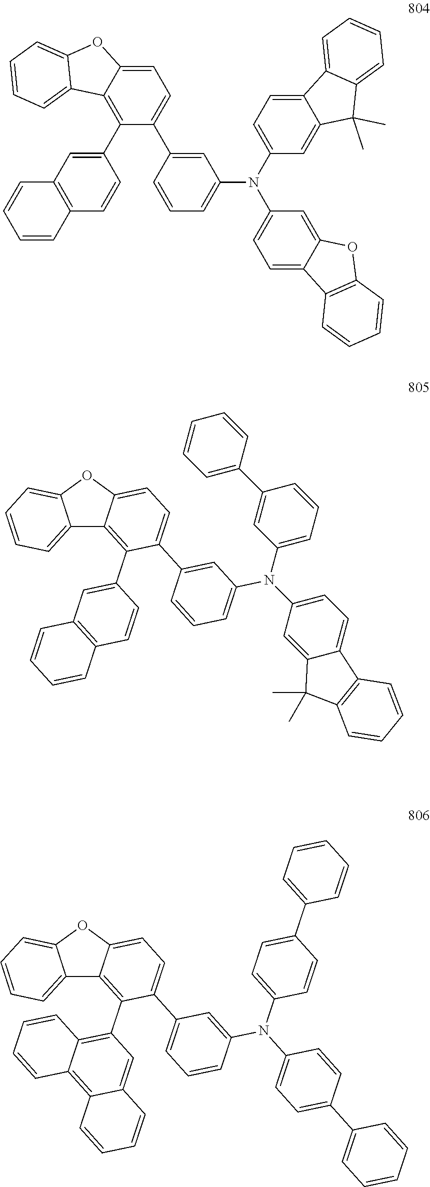 [SMILES: CC1(C)c2ccccc2-c2ccc(N(c3cccc(-c4ccc5oc6ccccc6c5c4-c4ccc5ccccc5c4)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4ccc5ccccc5c4)c3)cc21 ; c1ccc(-c2ccc(N(c3ccc(-c4ccccc4)cc3)c3cccc(-c4ccc5oc6ccccc6c5c4-c4cc5ccccc5c5ccccc45)c3)cc2)cc1]

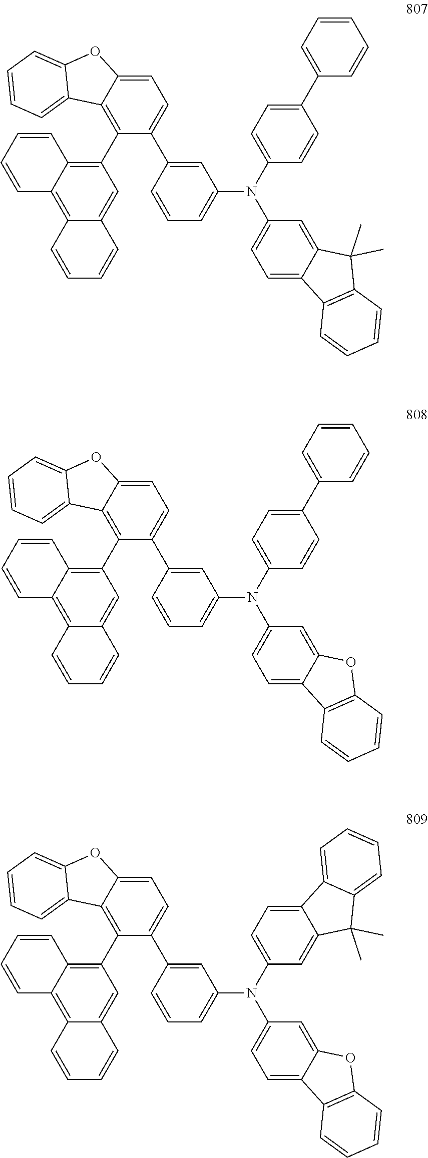 [SMILES: CC1(C)c2ccccc2-c2ccc(N(c3ccc(-c4ccccc4)cc3)c3cccc(-c4ccc5oc6ccccc6c5c4-c4cc5ccccc5c5ccccc45)c3)cc21 ; c1ccc(-c2ccc(N(c3cccc(-c4ccc5oc6ccccc6c5c4-c4cc5ccccc5c5ccccc45)c3)c3ccc4c(c3)oc3ccccc34)cc2)cc1 ; CC1(C)c2ccccc2-c2ccc(N(c3cccc(-c4ccc5oc6ccccc6c5c4-c4cc5ccccc5c5ccccc45)c3)c3ccc4c(c3)oc3ccccc34)cc21]

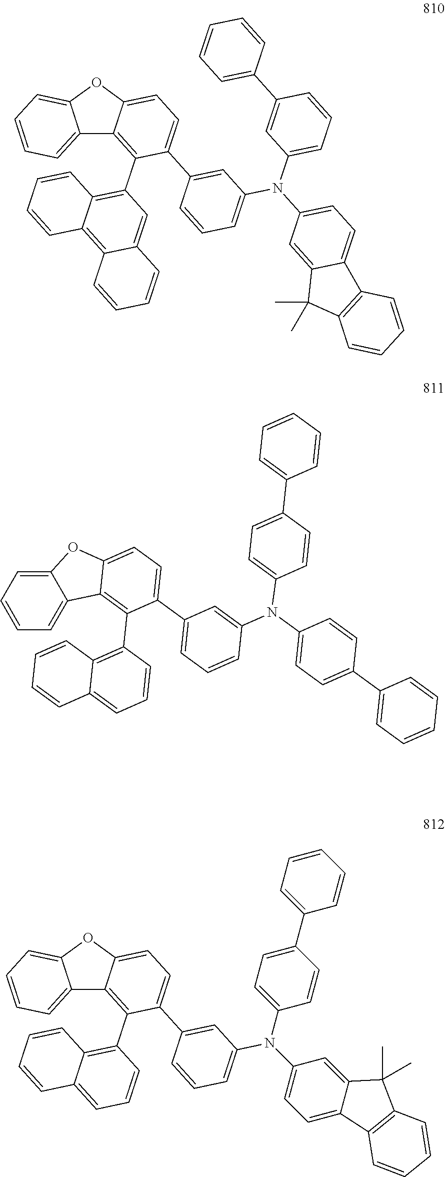

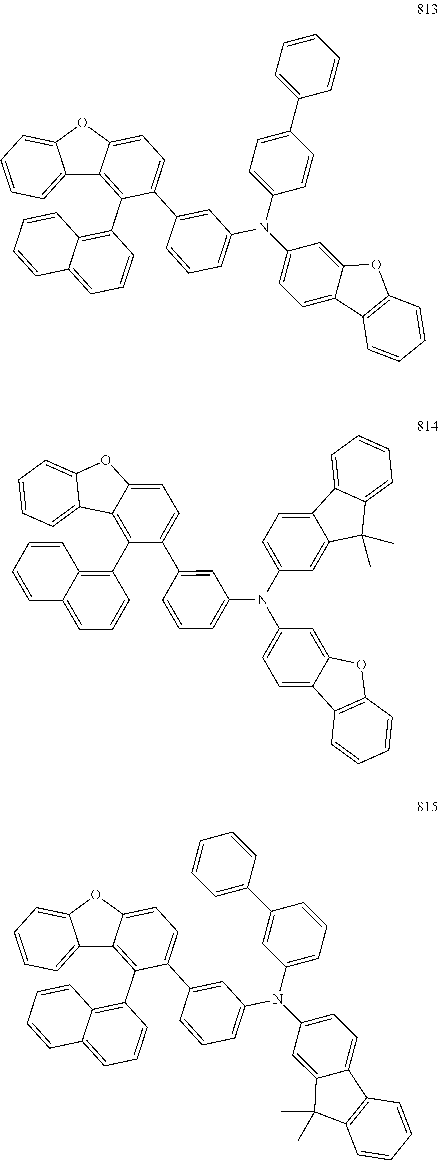 [SMILES: c1ccc(-c2ccc(N(c3cccc(-c4ccc5oc6ccccc6c5c4-c4cccc5ccccc45)c3)c3ccc4c(c3)oc3ccccc34)cc2)cc1 ; CC1(C)c2ccccc2-c2ccc(N(c3cccc(-c4ccc5oc6ccccc6c5c4-c4cccc5ccccc45)c3)c3ccc4c(c3)oc3ccccc34)cc21 ; CC1(C)c2ccccc2-c2ccc(N(c3cccc(-c4ccccc4)c3)c3cccc(-c4ccc5oc6ccccc6c5c4-c4cccc5ccccc45)c3)cc21]

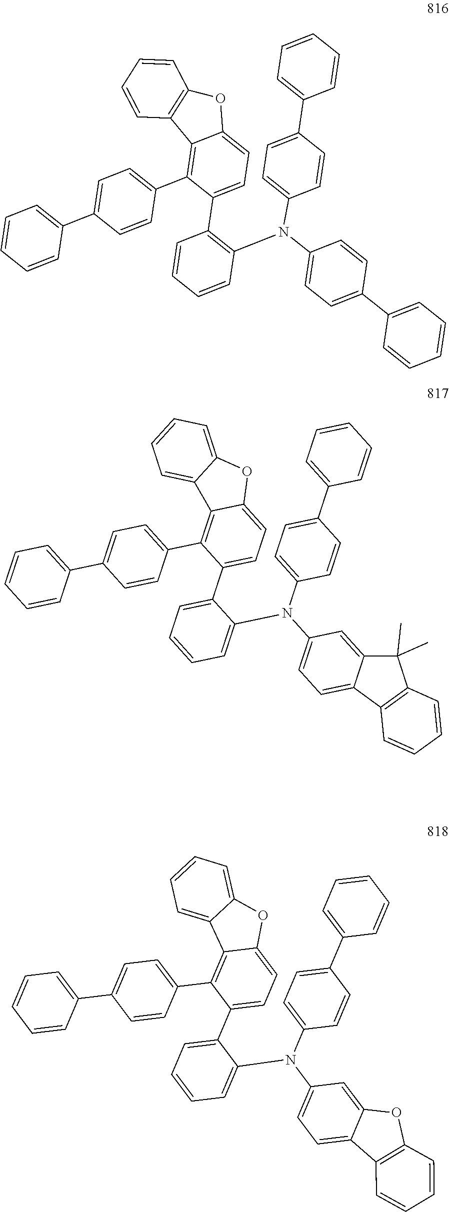 [SMILES: c1ccc(-c2ccc(-c3c(-c4ccccc4N(c4ccc(-c5ccccc5)cc4)c4ccc(-c5ccccc5)cc4)ccc4oc5ccccc5c34)cc2)cc1 ; CC1(C)c2ccccc2-c2ccc(N(c3ccc(-c4ccccc4)cc3)c3ccccc3-c3ccc4oc5ccccc5c4c3-c3ccc(-c4ccccc4)cc3)cc21 ; c1ccc(-c2ccc(-c3c(-c4ccccc4N(c4ccc(-c5ccccc5)cc4)c4ccc5c(c4)oc4ccccc45)ccc4oc5ccccc5c34)cc2)cc1]

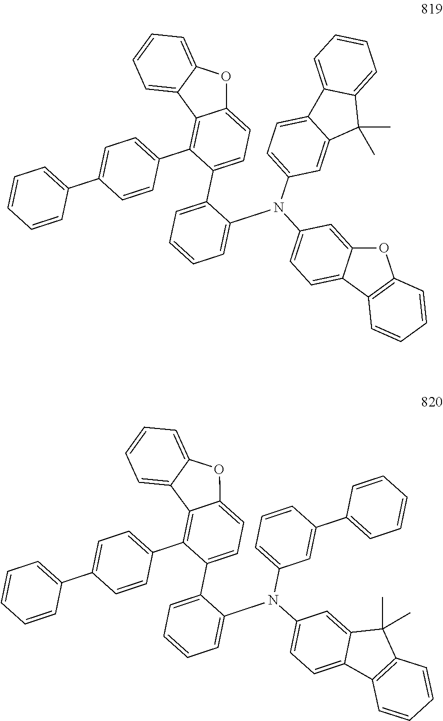 [SMILES: CC1(C)c2ccccc2-c2ccc(N(c3ccc4c(c3)oc3ccccc34)c3ccccc3-c3ccc4oc5ccccc5c4c3-c3ccc(-c4ccccc4)cc3)cc21 ; CC1(C)c2ccccc2-c2ccc(N(c3cccc(-c4ccccc4)c3)c3ccccc3-c3ccc4oc5ccccc5c4c3-c3ccc(-c4ccccc4)cc3)cc21]

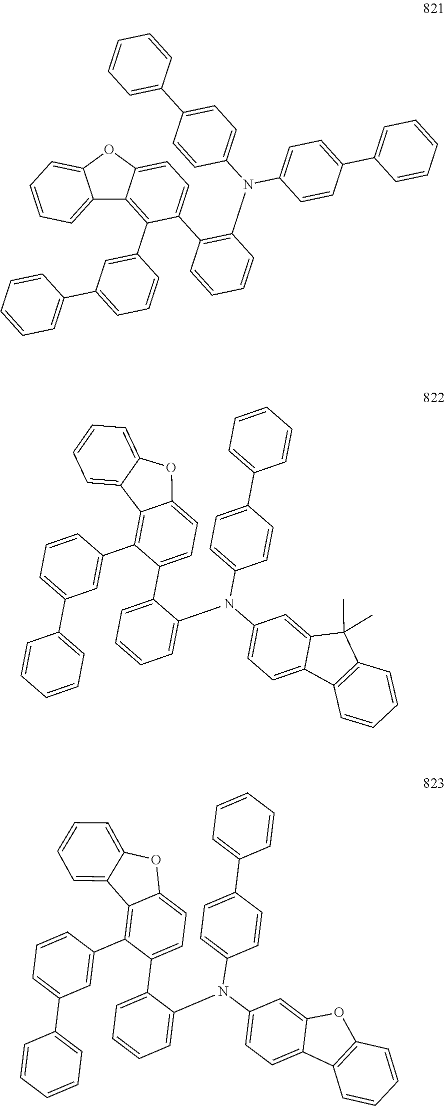 [SMILES: c1ccc(-c2ccc(N(c3ccc(-c4ccccc4)cc3)c3ccccc3-c3ccc4oc5ccccc5c4c3-c3cccc(-c4ccccc4)c3)cc2)cc1 ; CC1(C)c2ccccc2-c2ccc(N(c3ccc(-c4ccccc4)cc3)c3ccccc3-c3ccc4oc5ccccc5c4c3-c3cccc(-c4ccccc4)c3)cc21 ; c1ccc(-c2ccc(N(c3ccc4c(c3)oc3ccccc34)c3ccccc3-c3ccc4oc5ccccc5c4c3-c3cccc(-c4ccccc4)c3)cc2)cc1]

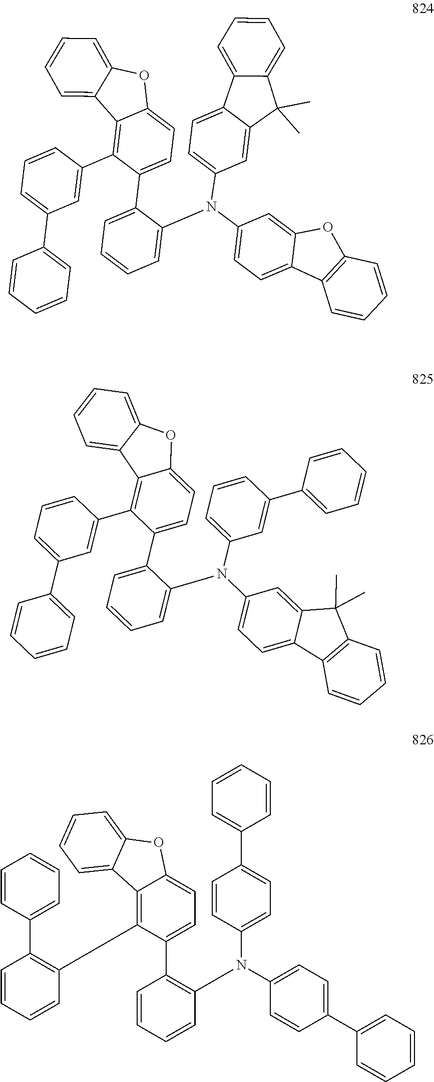 [SMILES: CC1(C)c2ccccc2-c2ccc(N(c3ccc4c(c3)oc3ccccc34)c3ccccc3-c3ccc4oc5ccccc5c4c3-c3cccc(-c4ccccc4)c3)cc21 ; CC1(C)c2ccccc2-c2ccc(N(c3cccc(-c4ccccc4)c3)c3ccccc3-c3ccc4oc5ccccc5c4c3-c3cccc(-c4ccccc4)c3)cc21 ; c1ccc(-c2ccc(N(c3ccc(-c4ccccc4)cc3)c3ccccc3-c3ccc4oc5ccccc5c4c3-c3ccccc3-c3ccccc3)cc2)cc1]

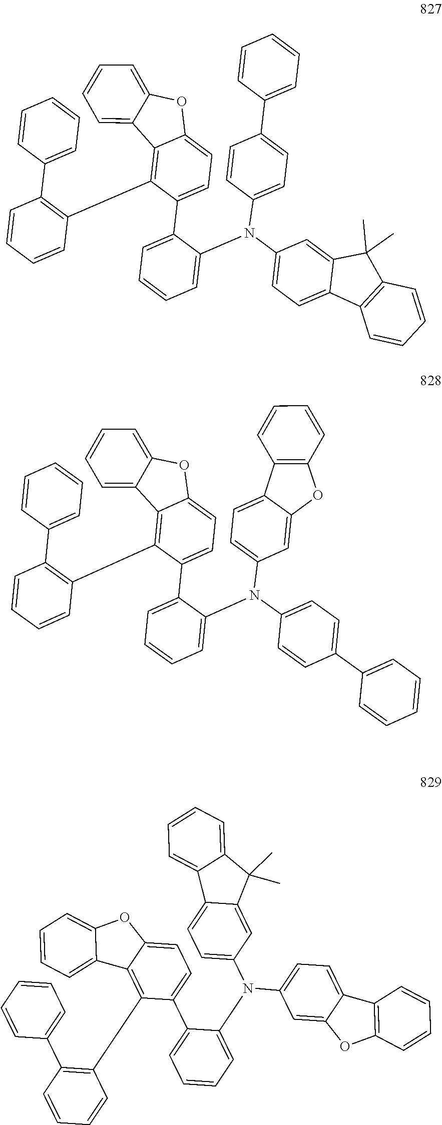 [SMILES: CC1(C)c2ccccc2-c2ccc(N(c3ccc(-c4ccccc4)cc3)c3ccccc3-c3ccc4oc5ccccc5c4c3-c3ccccc3-c3ccccc3)cc21 ; c1ccc(-c2ccc(N(c3ccc4c(c3)oc3ccccc34)c3ccccc3-c3ccc4oc5ccccc5c4c3-c3ccccc3-c3ccccc3)cc2)cc1 ; CC1(C)c2ccccc2-c2ccc(N(c3ccc4c(c3)oc3ccccc34)c3ccccc3-c3ccc4oc5ccccc5c4c3-c3ccccc3-c3ccccc3)cc21]

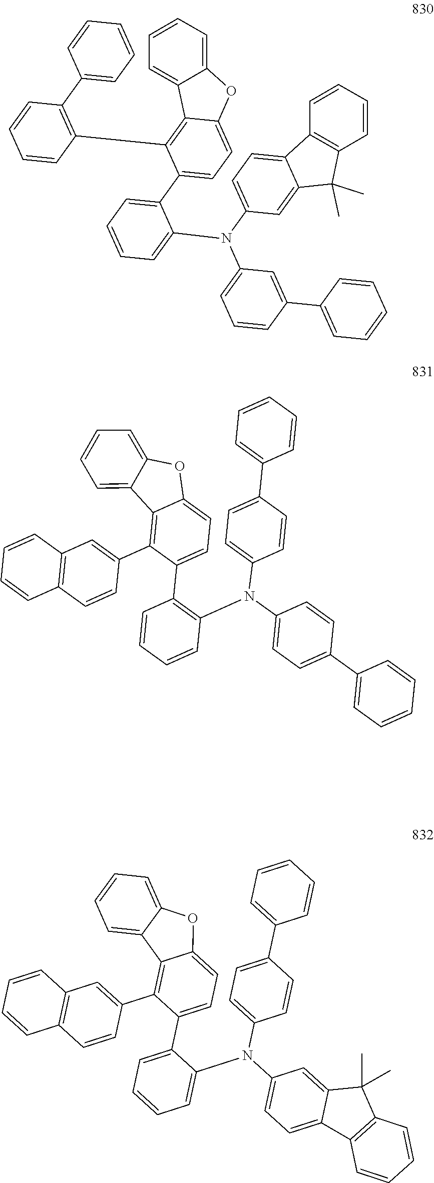 [SMILES: CC1(C)c2ccccc2-c2ccc(N(c3cccc(-c4ccccc4)c3)c3ccccc3-c3ccc4oc5ccccc5c4c3-c3ccccc3-c3ccccc3)cc21 ; c1ccc(-c2ccc(N(c3ccc(-c4ccccc4)cc3)c3ccccc3-c3ccc4oc5ccccc5c4c3-c3ccc4ccccc4c3)cc2)cc1 ; CC1(C)c2ccccc2-c2ccc(N(c3ccc(-c4ccccc4)cc3)c3ccccc3-c3ccc4oc5ccccc5c4c3-c3ccc4ccccc4c3)cc21]

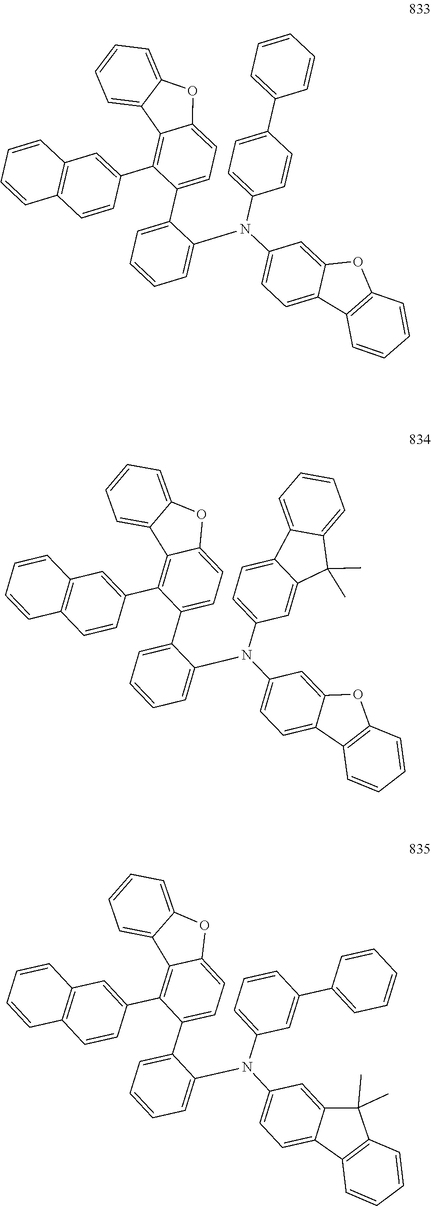 [SMILES: c1ccc(-c2ccc(N(c3ccc4c(c3)oc3ccccc34)c3ccccc3-c3ccc4oc5ccccc5c4c3-c3ccc4ccccc4c3)cc2)cc1 ; CC1(C)c2ccccc2-c2ccc(N(c3ccc4c(c3)oc3ccccc34)c3ccccc3-c3ccc4oc5ccccc5c4c3-c3ccc4ccccc4c3)cc21 ; CC1(C)c2ccccc2-c2ccc(N(c3cccc(-c4ccccc4)c3)c3ccccc3-c3ccc4oc5ccccc5c4c3-c3ccc4ccccc4c3)cc21]

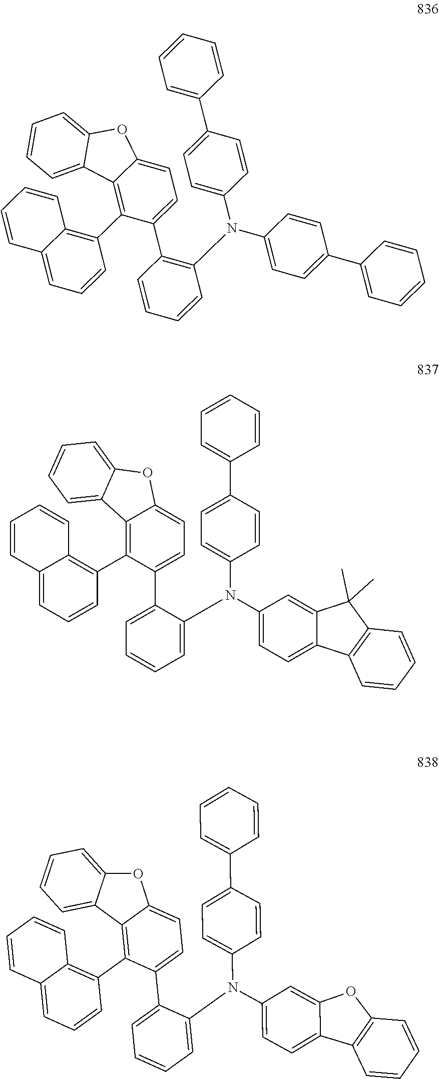 [SMILES: c1ccc(-c2ccc(N(c3ccc(-c4ccccc4)cc3)c3ccccc3-c3ccc4oc5ccccc5c4c3-c3cccc4ccccc34)cc2)cc1 ; CC1(C)c2ccccc2-c2ccc(N(c3ccc(-c4ccccc4)cc3)c3ccccc3-c3ccc4oc5ccccc5c4c3-c3cccc4ccccc34)cc21 ; c1ccc(-c2ccc(N(c3ccc4c(c3)oc3ccccc34)c3ccccc3-c3ccc4oc5ccccc5c4c3-c3cccc4ccccc34)cc2)cc1]

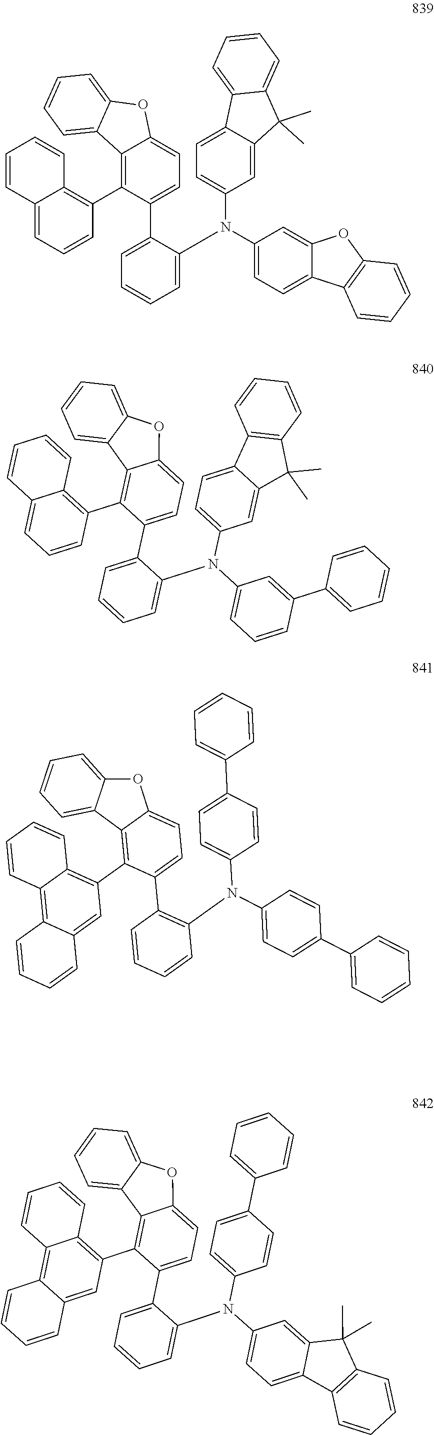 [SMILES: CC1(C)c2ccccc2-c2ccc(N(c3ccc4c(c3)oc3ccccc34)c3ccccc3-c3ccc4oc5ccccc5c4c3-c3cccc4ccccc34)cc21 ; CC1(C)c2ccccc2-c2ccc(N(c3cccc(-c4ccccc4)c3)c3ccccc3-c3ccc4oc5ccccc5c4c3-c3cccc4ccccc34)cc21 ; c1ccc(-c2ccc(N(c3ccc(-c4ccccc4)cc3)c3ccccc3-c3ccc4oc5ccccc5c4c3-c3cc4ccccc4c4ccccc34)cc2)cc1 ; CC1(C)c2ccccc2-c2ccc(N(c3ccc(-c4ccccc4)cc3)c3ccccc3-c3ccc4oc5ccccc5c4c3-c3cc4ccccc4c4ccccc34)cc21]

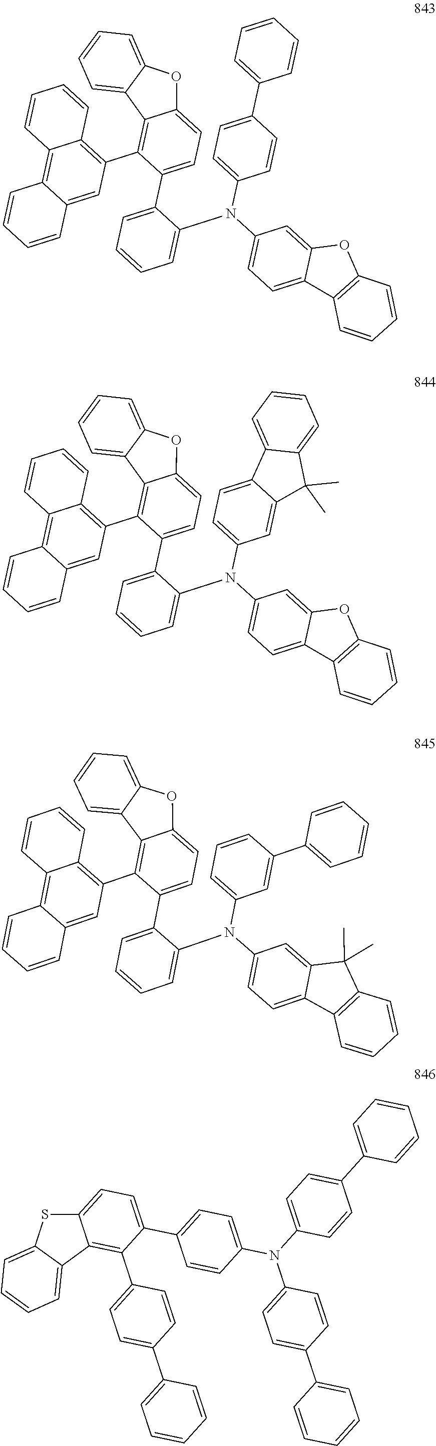

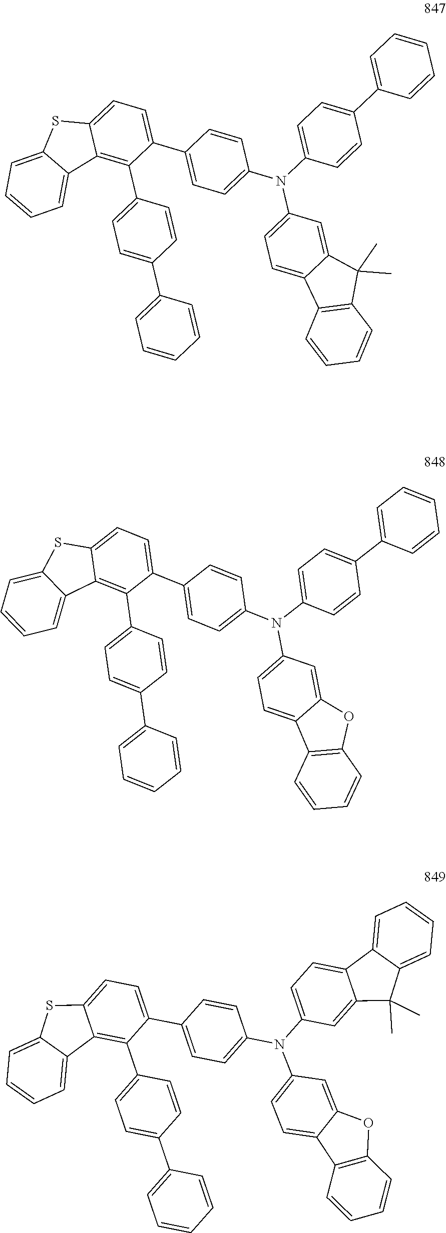 [SMILES: CC1(C)c2ccccc2-c2ccc(N(c3ccc(-c4ccccc4)cc3)c3ccc(-c4ccc5sc6ccccc6c5c4-c4ccc(-c5ccccc5)cc4)cc3)cc21 ; c1ccc(-c2ccc(-c3c(-c4ccc(N(c5ccc(-c6ccccc6)cc5)c5ccc6c(c5)oc5ccccc56)cc4)ccc4sc5ccccc5c34)cc2)cc1 ; CC1(C)c2ccccc2-c2ccc(N(c3ccc(-c4ccc5sc6ccccc6c5c4-c4ccc(-c5ccccc5)cc4)cc3)c3ccc4c(c3)oc3ccccc34)cc21]

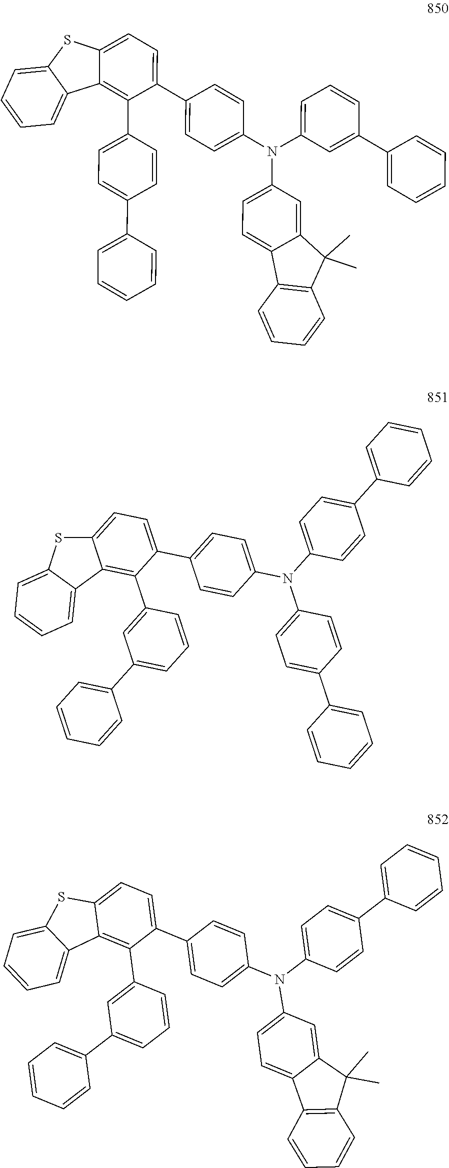 [SMILES: CC1(C)c2ccccc2-c2ccc(N(c3ccc(-c4ccc5sc6ccccc6c5c4-c4ccc(-c5ccccc5)cc4)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4cccc(-c5ccccc5)c4)cc3)cc2)cc1 ; CC1(C)c2ccccc2-c2ccc(N(c3ccc(-c4ccccc4)cc3)c3ccc(-c4ccc5sc6ccccc6c5c4-c4cccc(-c5ccccc5)c4)cc3)cc21]

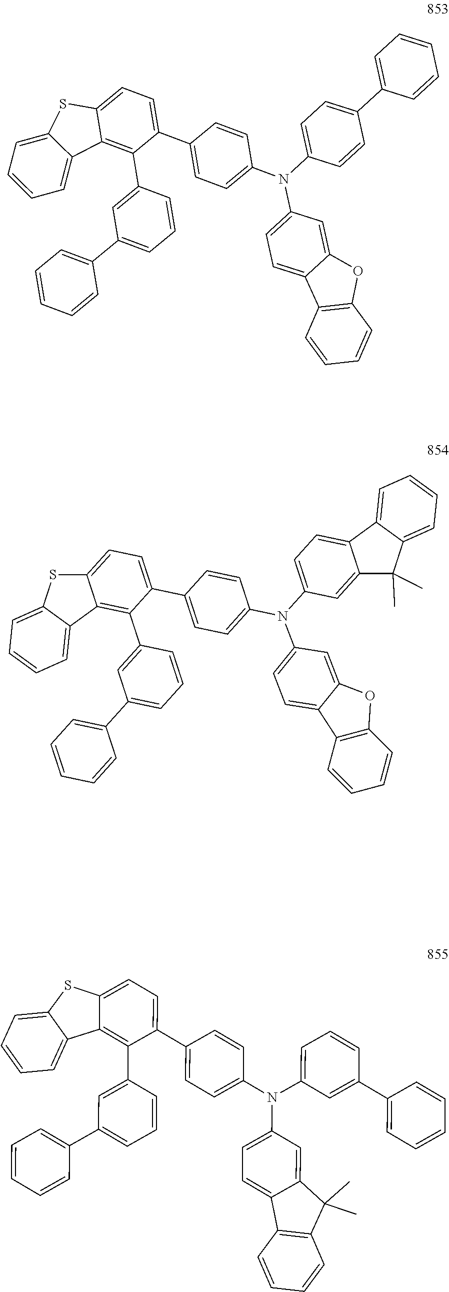 [SMILES: c1ccc(-c2ccc(N(c3ccc(-c4ccc5sc6ccccc6c5c4-c4cccc(-c5ccccc5)c4)cc3)c3ccc4c(c3)oc3ccccc34)cc2)cc1 ; CC1(C)c2ccccc2-c2ccc(N(c3ccc(-c4ccc5sc6ccccc6c5c4-c4cccc(-c5ccccc5)c4)cc3)c3ccc4c(c3)oc3ccccc34)cc21 ; CC1(C)c2ccccc2-c2ccc(N(c3ccc(-c4ccc5sc6ccccc6c5c4-c4cccc(-c5ccccc5)c4)cc3)c3cccc(-c4ccccc4)c3)cc21]

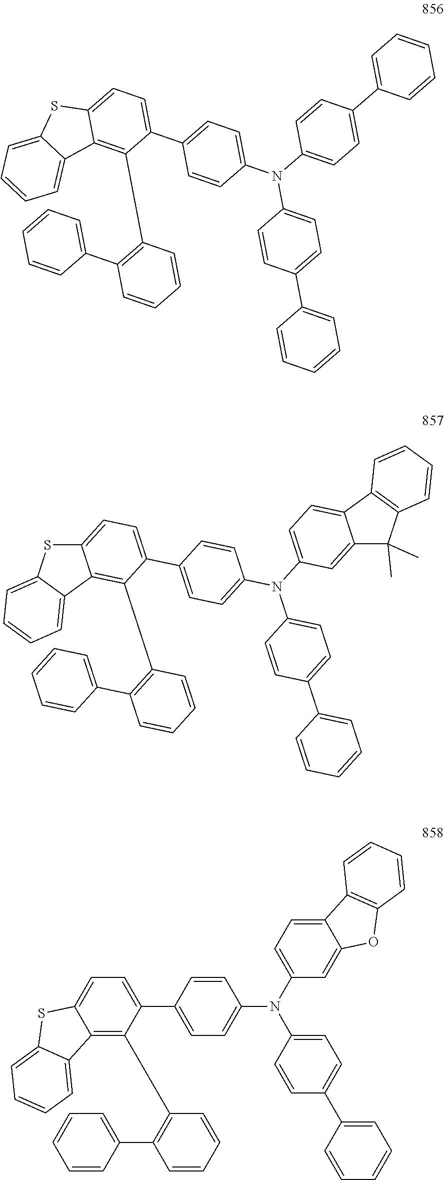 [SMILES: c1ccc(-c2ccc(N(c3ccc(-c4ccccc4)cc3)c3ccc(-c4ccc5sc6ccccc6c5c4-c4ccccc4-c4ccccc4)cc3)cc2)cc1 ; CC1(C)c2ccccc2-c2ccc(N(c3ccc(-c4ccccc4)cc3)c3ccc(-c4ccc5sc6ccccc6c5c4-c4ccccc4-c4ccccc4)cc3)cc21 ; c1ccc(-c2ccc(N(c3ccc(-c4ccc5sc6ccccc6c5c4-c4ccccc4-c4ccccc4)cc3)c3ccc4c(c3)oc3ccccc34)cc2)cc1]

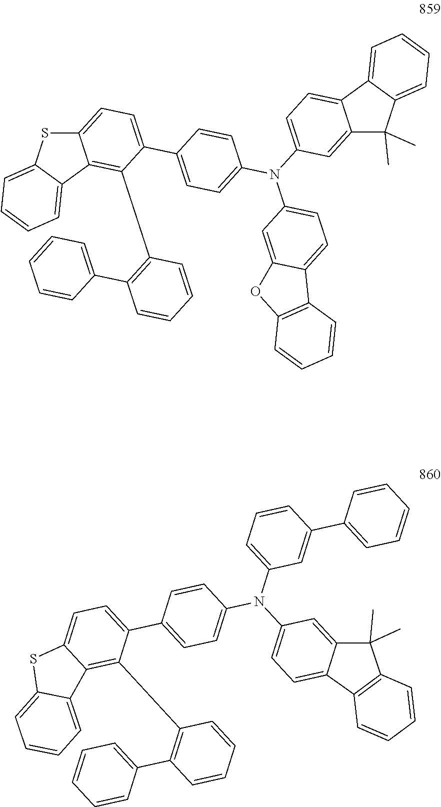 [SMILES: CC1(C)c2ccccc2-c2ccc(N(c3ccc(-c4ccc5sc6ccccc6c5c4-c4ccccc4-c4ccccc4)cc3)c3ccc4c(c3)oc3ccccc34)cc21 ; CC1(C)c2ccccc2-c2ccc(N(c3ccc(-c4ccc5sc6ccccc6c5c4-c4ccccc4-c4ccccc4)cc3)c3cccc(-c4ccccc4)c3)cc21]

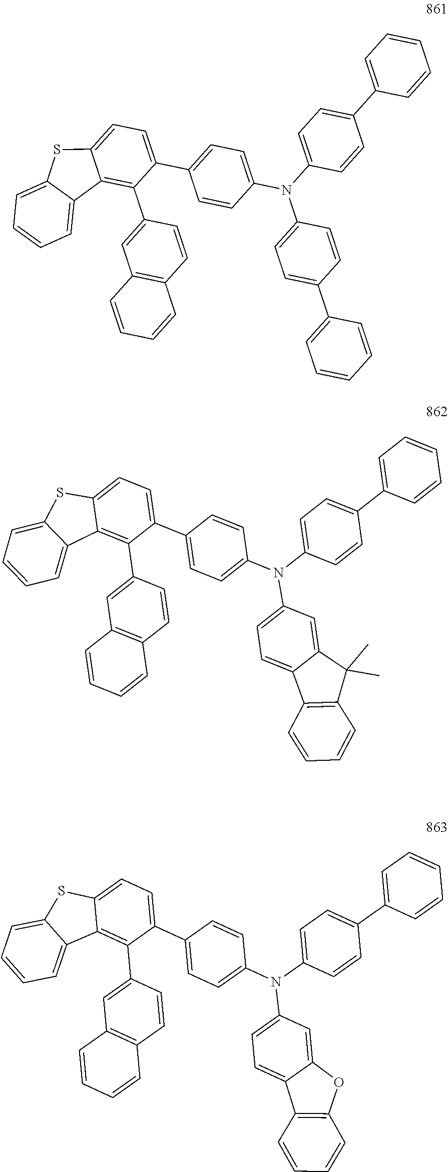 [SMILES: c1ccc(-c2ccc(N(c3ccc(-c4ccccc4)cc3)c3ccc(-c4ccc5sc6ccccc6c5c4-c4ccc5ccccc5c4)cc3)cc2)cc1 ; CC1(C)c2ccccc2-c2ccc(N(c3ccc(-c4ccccc4)cc3)c3ccc(-c4ccc5sc6ccccc6c5c4-c4ccc5ccccc5c4)cc3)cc21 ; c1ccc(-c2ccc(N(c3ccc(-c4ccc5sc6ccccc6c5c4-c4ccc5ccccc5c4)cc3)c3ccc4c(c3)oc3ccccc34)cc2)cc1]

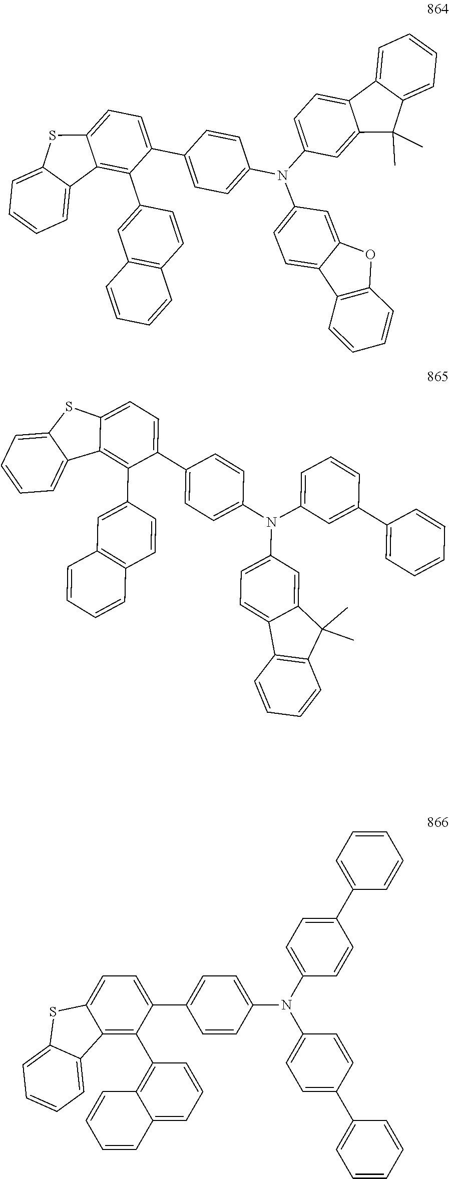 [SMILES: CC1(C)c2ccccc2-c2ccc(N(c3ccc(-c4ccc5sc6ccccc6c5c4-c4ccc5ccccc5c4)cc3)c3ccc4c(c3)oc3ccccc34)cc21 ; CC1(C)c2ccccc2-c2ccc(N(c3ccc(-c4ccc5sc6ccccc6c5c4-c4ccc5ccccc5c4)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4cccc5ccccc45)cc3)cc2)cc1]

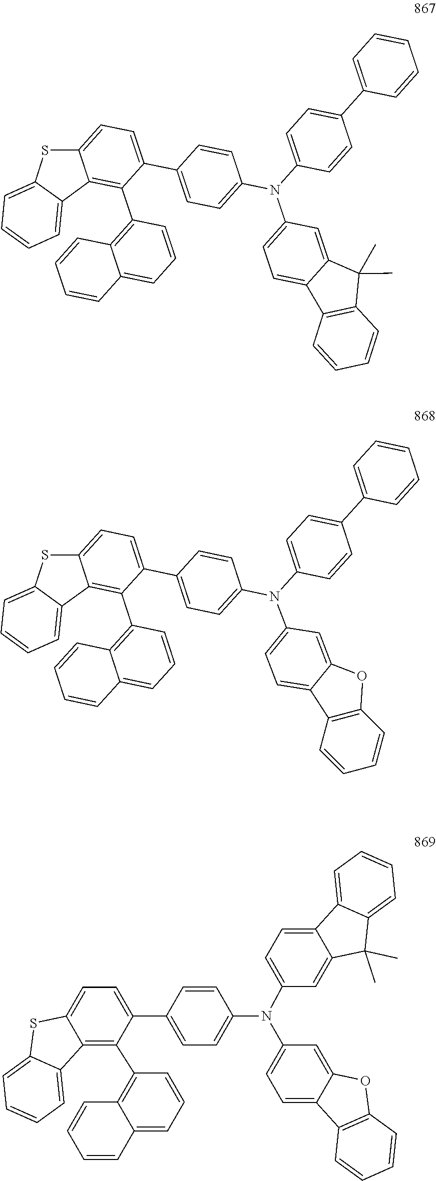 [SMILES: CC1(C)c2ccccc2-c2ccc(N(c3ccc(-c4ccccc4)cc3)c3ccc(-c4ccc5sc6ccccc6c5c4-c4cccc5ccccc45)cc3)cc21 ; c1ccc(-c2ccc(N(c3ccc(-c4ccc5sc6ccccc6c5c4-c4cccc5ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4cccc5ccccc45)cc3)c3ccc4c(c3)oc3ccccc34)cc21]

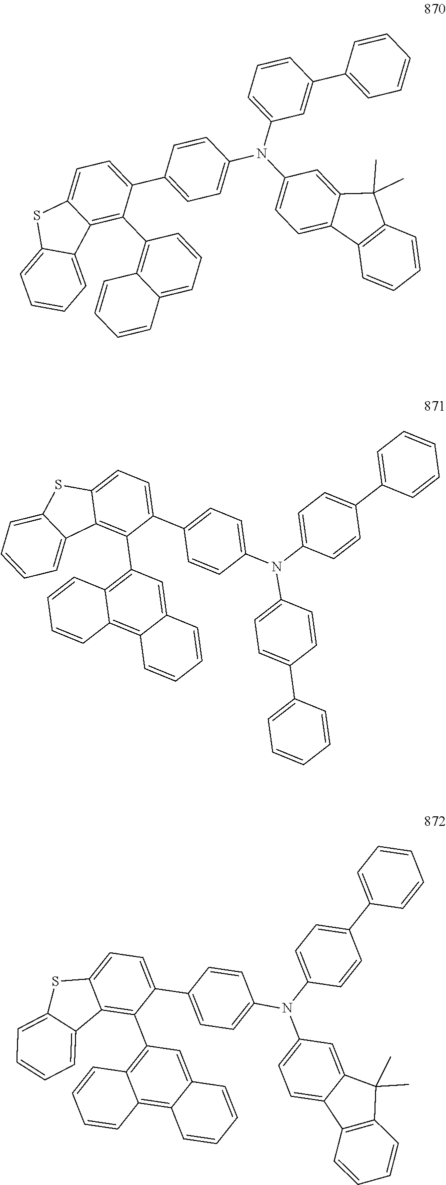 [SMILES: CC1(C)c2ccccc2-c2ccc(N(c3ccc(-c4ccc5sc6ccccc6c5c4-c4cccc5ccccc45)cc3)c3cccc(-c4ccccc4)c3)cc21 ; c1ccc(-c2ccc(N(c3ccc(-c4ccccc4)cc3)c3ccc(-c4ccc5sc6ccccc6c5c4-c4cc5ccccc5c5ccccc45)cc3)cc2)cc1 ; CC1(C)c2ccccc2-c2ccc(N(c3ccc(-c4ccccc4)cc3)c3ccc(-c4ccc5sc6ccccc6c5c4-c4cc5ccccc5c5ccccc45)cc3)cc21]

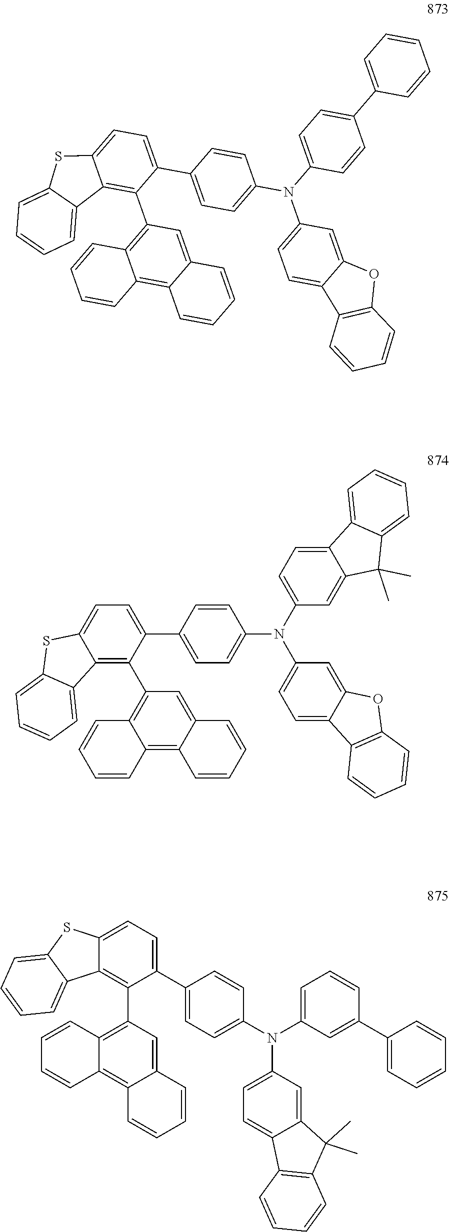 [SMILES: c1ccc(-c2ccc(N(c3ccc(-c4ccc5sc6ccccc6c5c4-c4cc5ccccc5c5ccccc45)cc3)c3ccc4c(c3)oc3ccccc34)cc2)cc1 ; CC1(C)c2ccccc2-c2ccc(N(c3ccc(-c4ccc5sc6ccccc6c5c4-c4cc5ccccc5c5ccccc45)cc3)c3ccc4c(c3)oc3ccccc34)cc21 ; CC1(C)c2ccccc2-c2ccc(N(c3ccc(-c4ccc5sc6ccccc6c5c4-c4cc5ccccc5c5ccccc45)cc3)c3cccc(-c4ccccc4)c3)cc21]

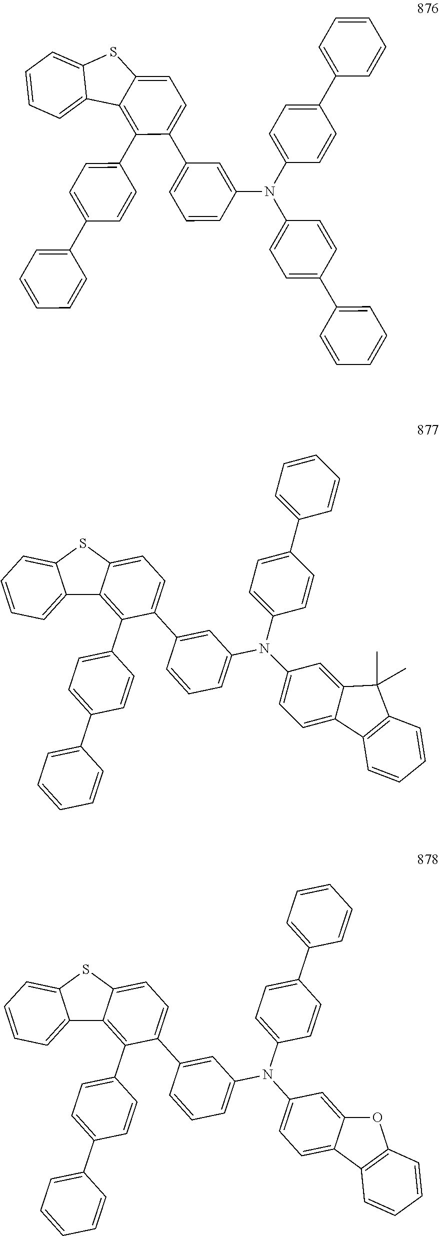 [SMILES: c1ccc(-c2ccc(-c3c(-c4cccc(N(c5ccc(-c6ccccc6)cc5)c5ccc(-c6ccccc6)cc5)c4)ccc4sc5ccccc5c34)cc2)cc1 ; CC1(C)c2ccccc2-c2ccc(N(c3ccc(-c4ccccc4)cc3)c3cccc(-c4ccc5sc6ccccc6c5c4-c4ccc(-c5ccccc5)cc4)c3)cc21 ; c1ccc(-c2ccc(-c3c(-c4cccc(N(c5ccc(-c6ccccc6)cc5)c5ccc6c(c5)oc5ccccc56)c4)ccc4sc5ccccc5c34)cc2)cc1]

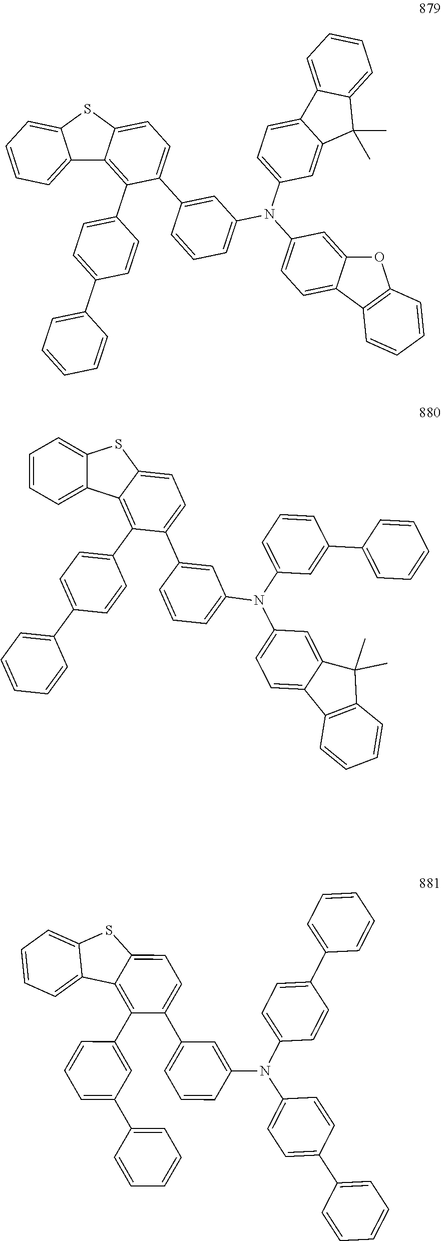 [SMILES: CC1(C)c2ccccc2-c2ccc(N(c3cccc(-c4ccc5sc6ccccc6c5c4-c4ccc(-c5ccccc5)cc4)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4ccc(-c5ccccc5)cc4)c3)cc21 ; c1ccc(-c2ccc(N(c3ccc(-c4ccccc4)cc3)c3cccc(-c4ccc5sc6ccccc6c5c4-c4cccc(-c5ccccc5)c4)c3)cc2)cc1]

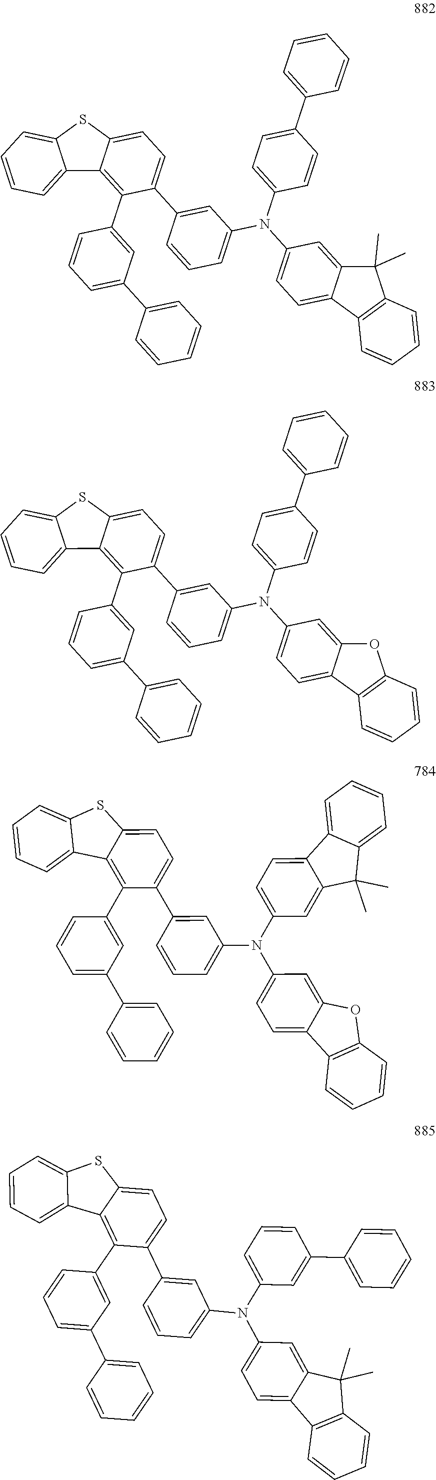 [SMILES: CC1(C)c2ccccc2-c2ccc(N(c3ccc(-c4ccccc4)cc3)c3cccc(-c4ccc5sc6ccccc6c5c4-c4cccc(-c5ccccc5)c4)c3)cc21 ; c1ccc(-c2ccc(N(c3cccc(-c4ccc5sc6ccccc6c5c4-c4cccc(-c5ccccc5)c4)c3)c3ccc4c(c3)oc3ccccc34)cc2)cc1 ; CC1(C)c2ccccc2-c2ccc(N(c3cccc(-c4ccc5sc6ccccc6c5c4-c4cccc(-c5ccccc5)c4)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4cccc(-c5ccccc5)c4)c3)cc21]

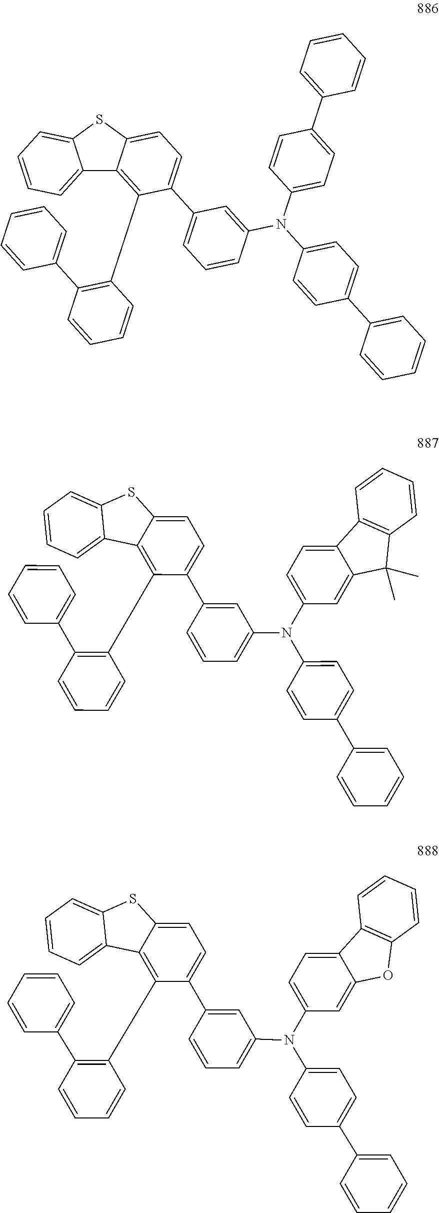 [SMILES: c1ccc(-c2ccc(N(c3ccc(-c4ccccc4)cc3)c3cccc(-c4ccc5sc6ccccc6c5c4-c4ccccc4-c4ccccc4)c3)cc2)cc1 ; CC1(C)c2ccccc2-c2ccc(N(c3ccc(-c4ccccc4)cc3)c3cccc(-c4ccc5sc6ccccc6c5c4-c4ccccc4-c4ccccc4)c3)cc21 ; c1ccc(-c2ccc(N(c3cccc(-c4ccc5sc6ccccc6c5c4-c4ccccc4-c4ccccc4)c3)c3ccc4c(c3)oc3ccccc34)cc2)cc1]

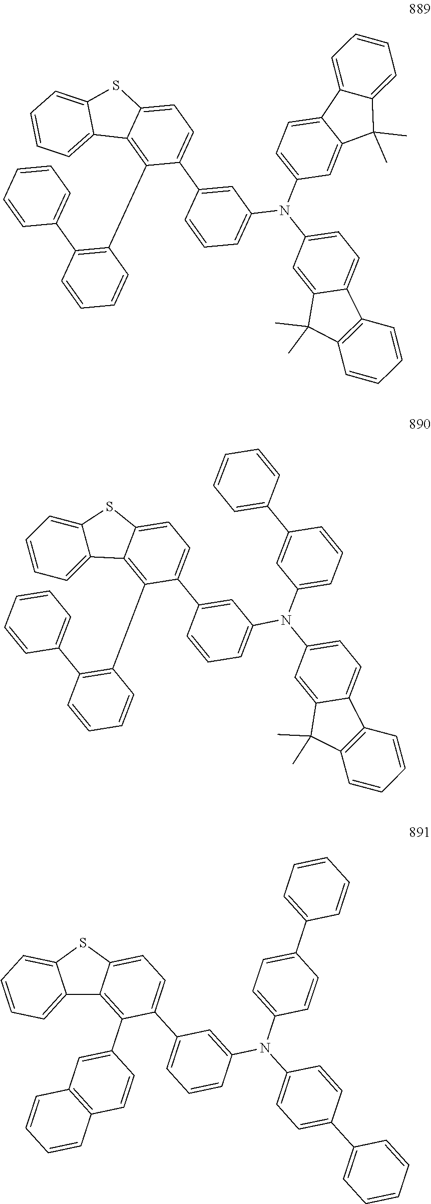 [SMILES: CC1(C)c2ccccc2-c2ccc(N(c3cccc(-c4ccc5sc6ccccc6c5c4-c4ccccc4-c4ccccc4)c3)c3ccc4c(c3)C(C)(C)c3ccccc3-4)cc21 ; CC1(C)c2ccccc2-c2ccc(N(c3cccc(-c4ccccc4)c3)c3cccc(-c4ccc5sc6ccccc6c5c4-c4ccccc4-c4ccccc4)c3)cc21 ; c1ccc(-c2ccc(N(c3ccc(-c4ccccc4)cc3)c3cccc(-c4ccc5sc6ccccc6c5c4-c4ccc5ccccc5c4)c3)cc2)cc1]

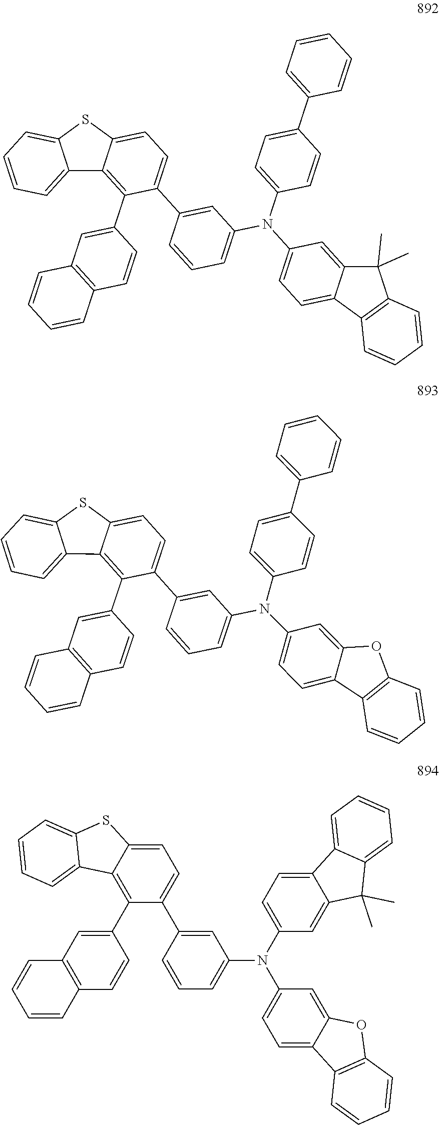 [SMILES: CC1(C)c2ccccc2-c2ccc(N(c3ccc(-c4ccccc4)cc3)c3cccc(-c4ccc5sc6ccccc6c5c4-c4ccc5ccccc5c4)c3)cc21 ; c1ccc(-c2ccc(N(c3cccc(-c4ccc5sc6ccccc6c5c4-c4ccc5ccccc5c4)c3)c3ccc4c(c3)oc3ccccc34)cc2)cc1 ; CC1(C)c2ccccc2-c2ccc(N(c3cccc(-c4ccc5sc6ccccc6c5c4-c4ccc5ccccc5c4)c3)c3ccc4c(c3)oc3ccccc34)cc21]

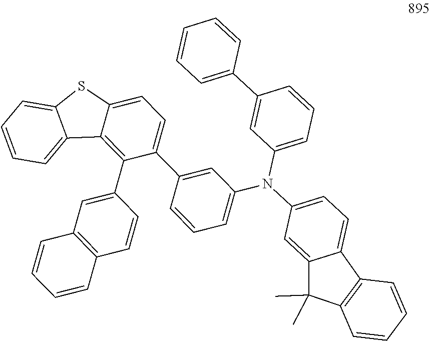 [SMILES: CC1(C)c2ccccc2-c2ccc(N(c3cccc(-c4ccccc4)c3)c3cccc(-c4ccc5sc6ccccc6c5c4-c4ccc5ccccc5c4)c3)cc21]

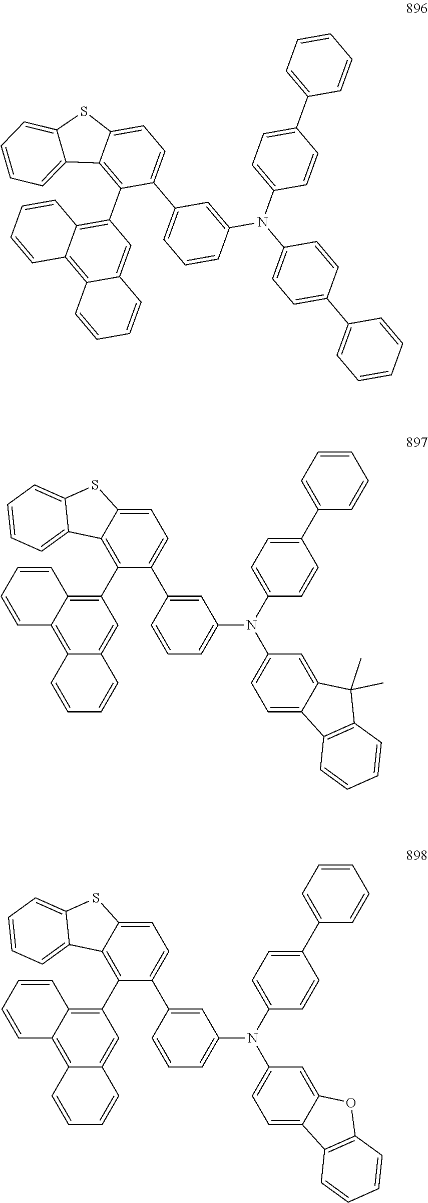 [SMILES: c1ccc(-c2ccc(N(c3ccc(-c4ccccc4)cc3)c3cccc(-c4ccc5sc6ccccc6c5c4-c4cc5ccccc5c5ccccc45)c3)cc2)cc1 ; CC1(C)c2ccccc2-c2ccc(N(c3ccc(-c4ccccc4)cc3)c3cccc(-c4ccc5sc6ccccc6c5c4-c4cc5ccccc5c5ccccc45)c3)cc21 ; c1ccc(-c2ccc(N(c3cccc(-c4ccc5sc6ccccc6c5c4-c4cc5ccccc5c5ccccc45)c3)c3ccc4c(c3)oc3ccccc34)cc2)cc1]

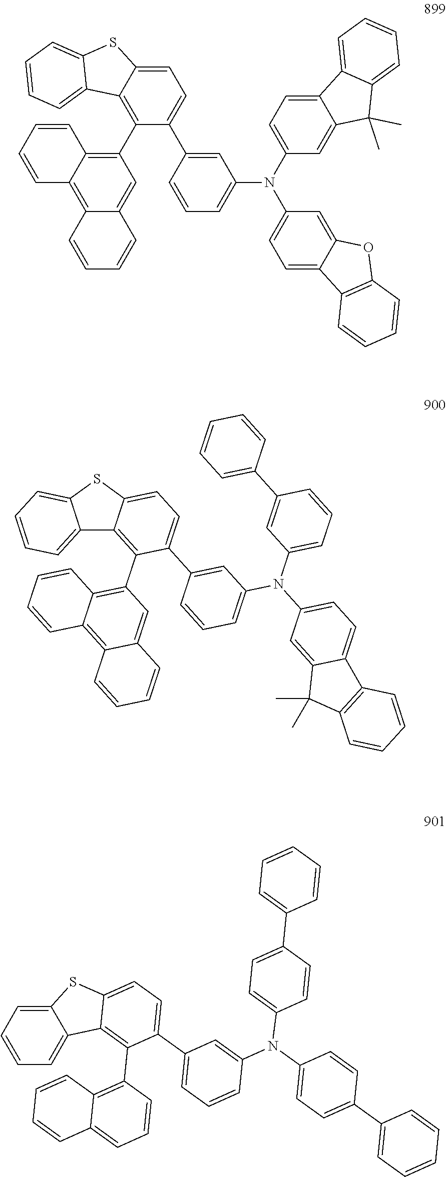 [SMILES: CC1(C)c2ccccc2-c2ccc(N(c3cccc(-c4ccc5sc6ccccc6c5c4-c4cc5ccccc5c5ccccc45)c3)c3ccc4c(c3)oc3ccccc34)cc21 ; CC1(C)c2ccccc2-c2ccc(N(c3cccc(-c4ccccc4)c3)c3cccc(-c4ccc5sc6ccccc6c5c4-c4cc5ccccc5c5ccccc45)c3)cc21 ; c1ccc(-c2ccc(N(c3ccc(-c4ccccc4)cc3)c3cccc(-c4ccc5sc6ccccc6c5c4-c4cccc5ccccc45)c3)cc2)cc1]

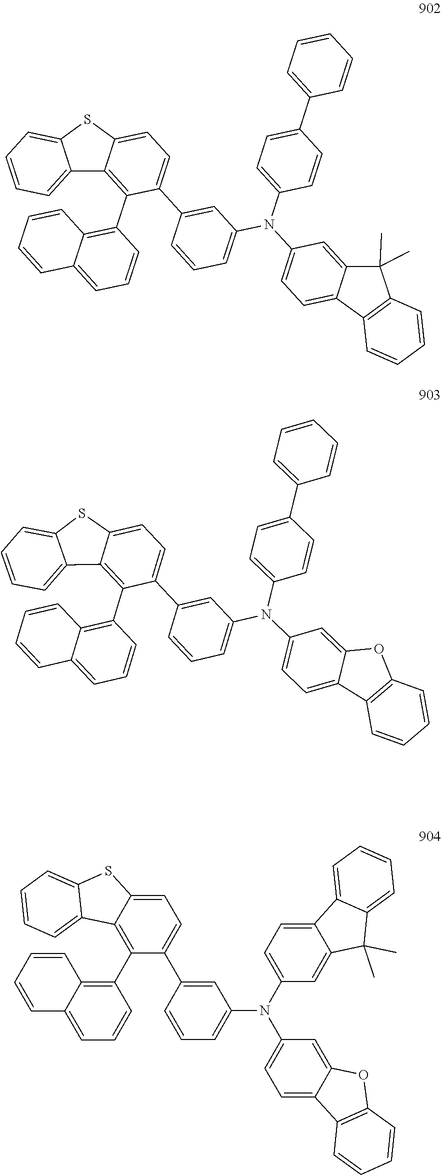 [SMILES: CC1(C)c2ccccc2-c2ccc(N(c3ccc(-c4ccccc4)cc3)c3cccc(-c4ccc5sc6ccccc6c5c4-c4cccc5ccccc45)c3)cc21 ; c1ccc(-c2ccc(N(c3cccc(-c4ccc5sc6ccccc6c5c4-c4cccc5ccccc45)c3)c3ccc4c(c3)oc3ccccc34)cc2)cc1 ; CC1(C)c2ccccc2-c2ccc(N(c3cccc(-c4ccc5sc6ccccc6c5c4-c4cccc5ccccc45)c3)c3ccc4c(c3)oc3ccccc34)cc21]

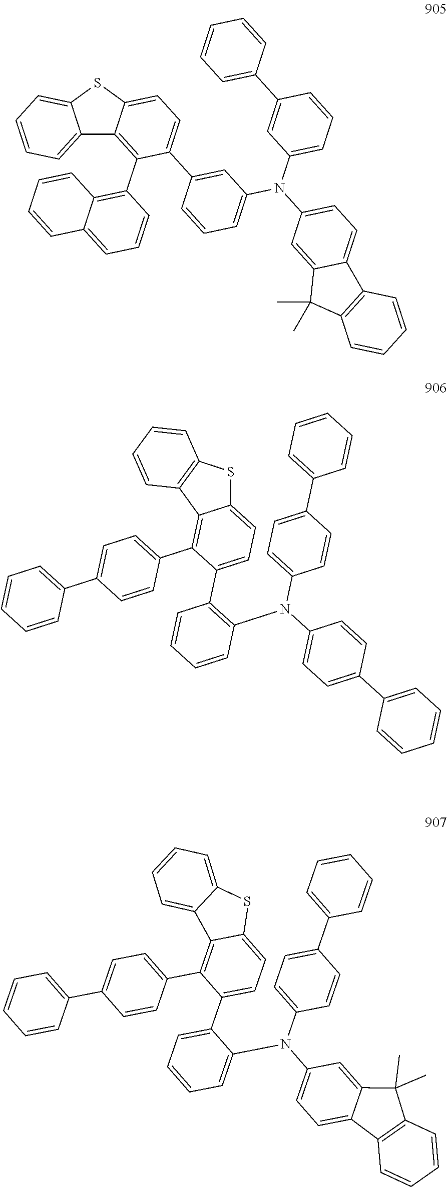 [SMILES: CC1(C)c2ccccc2-c2ccc(N(c3cccc(-c4ccccc4)c3)c3cccc(-c4ccc5sc6ccccc6c5c4-c4cccc5ccccc45)c3)cc21 ; c1ccc(-c2ccc(-c3c(-c4ccccc4N(c4ccc(-c5ccccc5)cc4)c4ccc(-c5ccccc5)cc4)ccc4sc5ccccc5c34)cc2)cc1 ; CC1(C)c2ccccc2-c2ccc(N(c3ccc(-c4ccccc4)cc3)c3ccccc3-c3ccc4sc5ccccc5c4c3-c3ccc(-c4ccccc4)cc3)cc21]

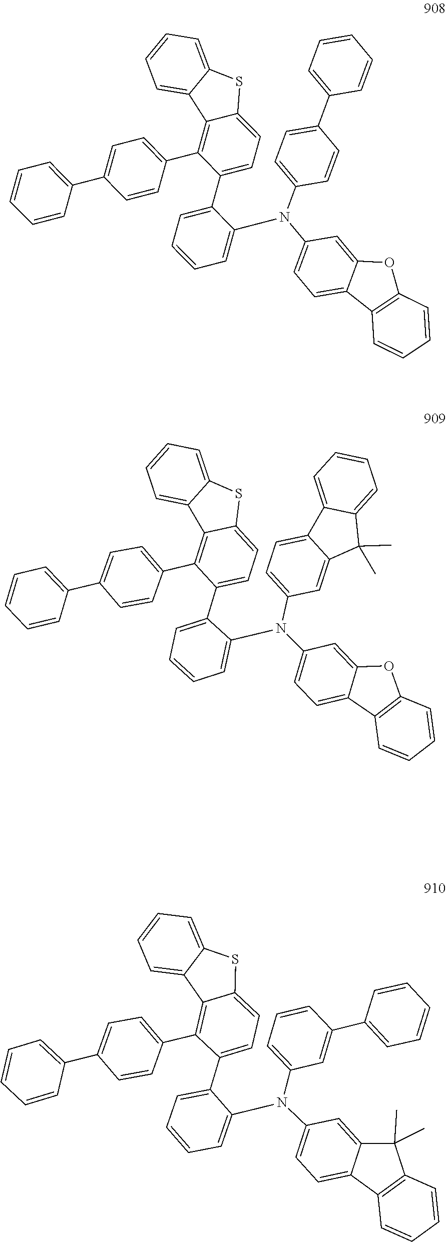 [SMILES: c1ccc(-c2ccc(-c3c(-c4ccccc4N(c4ccc(-c5ccccc5)cc4)c4ccc5c(c4)oc4ccccc45)ccc4sc5ccccc5c34)cc2)cc1 ; CC1(C)c2ccccc2-c2ccc(N(c3ccc4c(c3)oc3ccccc34)c3ccccc3-c3ccc4sc5ccccc5c4c3-c3ccc(-c4ccccc4)cc3)cc21 ; CC1(C)c2ccccc2-c2ccc(N(c3cccc(-c4ccccc4)c3)c3ccccc3-c3ccc4sc5ccccc5c4c3-c3ccc(-c4ccccc4)cc3)cc21]

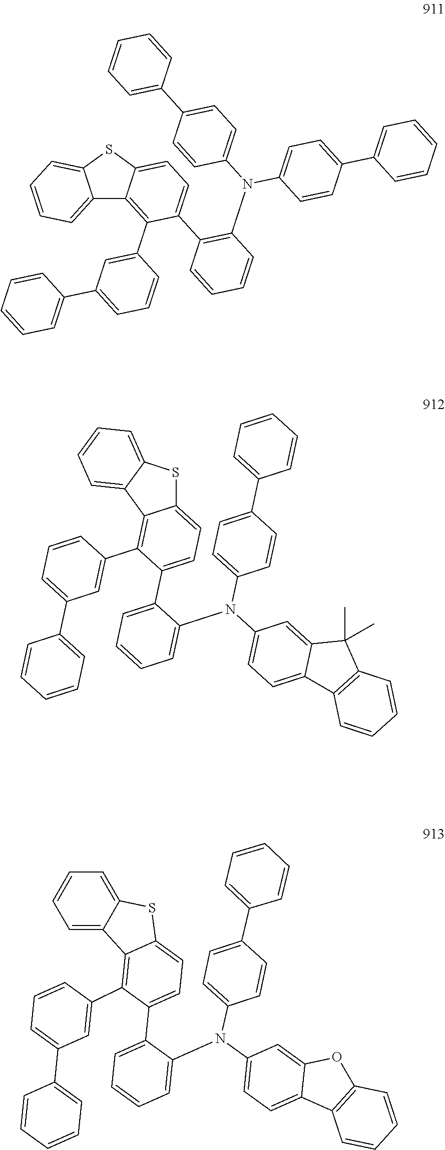 [SMILES: c1ccc(-c2ccc(N(c3ccc(-c4ccccc4)cc3)c3ccccc3-c3ccc4sc5ccccc5c4c3-c3cccc(-c4ccccc4)c3)cc2)cc1 ; CC1(C)c2ccccc2-c2ccc(N(c3ccc(-c4ccccc4)cc3)c3ccccc3-c3ccc4sc5ccccc5c4c3-c3cccc(-c4ccccc4)c3)cc21 ; c1ccc(-c2ccc(N(c3ccc4c(c3)oc3ccccc34)c3ccccc3-c3ccc4sc5ccccc5c4c3-c3cccc(-c4ccccc4)c3)cc2)cc1]

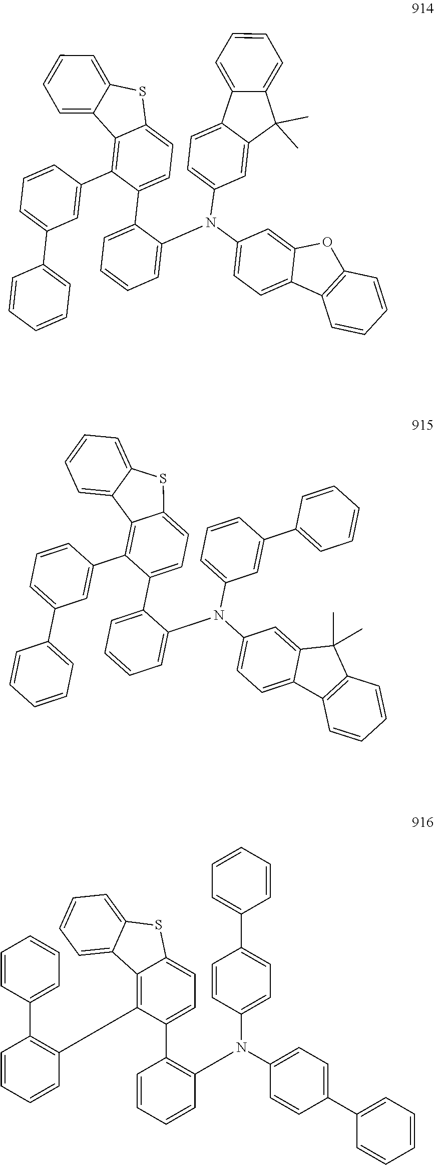 [SMILES: CC1(C)c2ccccc2-c2ccc(N(c3ccc4c(c3)oc3ccccc34)c3ccccc3-c3ccc4sc5ccccc5c4c3-c3cccc(-c4ccccc4)c3)cc21 ; CC1(C)c2ccccc2-c2ccc(N(c3cccc(-c4ccccc4)c3)c3ccccc3-c3ccc4sc5ccccc5c4c3-c3cccc(-c4ccccc4)c3)cc21 ; c1ccc(-c2ccc(N(c3ccc(-c4ccccc4)cc3)c3ccccc3-c3ccc4sc5ccccc5c4c3-c3ccccc3-c3ccccc3)cc2)cc1]

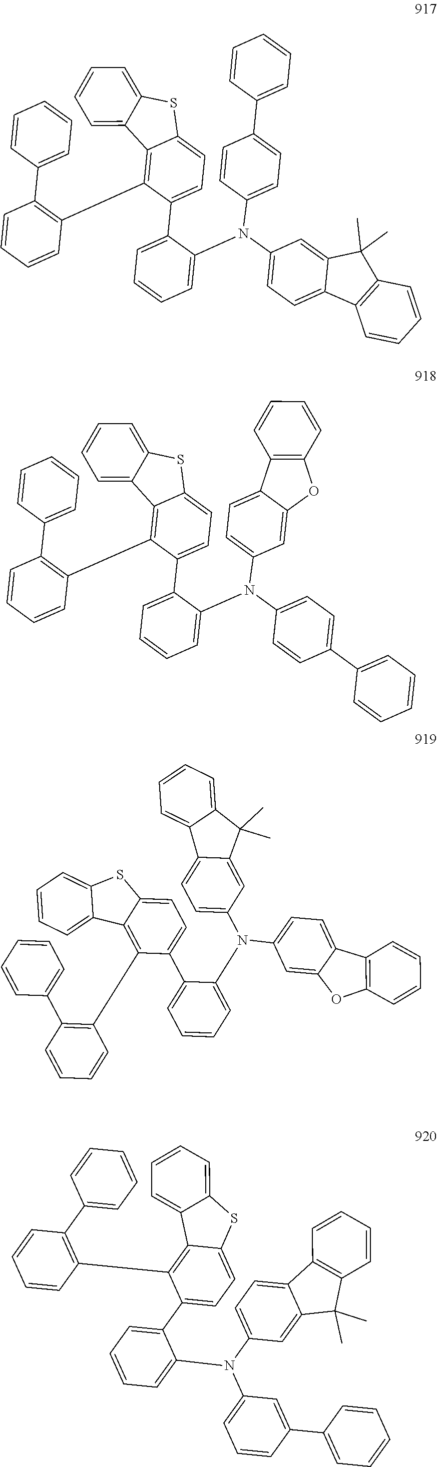 [SMILES: CC1(C)c2ccccc2-c2ccc(N(c3ccc(-c4ccccc4)cc3)c3ccccc3-c3ccc4sc5ccccc5c4c3-c3ccccc3-c3ccccc3)cc21 ; c1ccc(-c2ccc(N(c3ccc4c(c3)oc3ccccc34)c3ccccc3-c3ccc4sc5ccccc5c4c3-c3ccccc3-c3ccccc3)cc2)cc1 ; CC1(C)c2ccccc2-c2ccc(N(c3ccc4c(c3)oc3ccccc34)c3ccccc3-c3ccc4sc5ccccc5c4c3-c3ccccc3-c3ccccc3)cc21 ; CC1(C)c2ccccc2-c2ccc(N(c3cccc(-c4ccccc4)c3)c3ccccc3-c3ccc4sc5ccccc5c4c3-c3ccccc3-c3ccccc3)cc21]

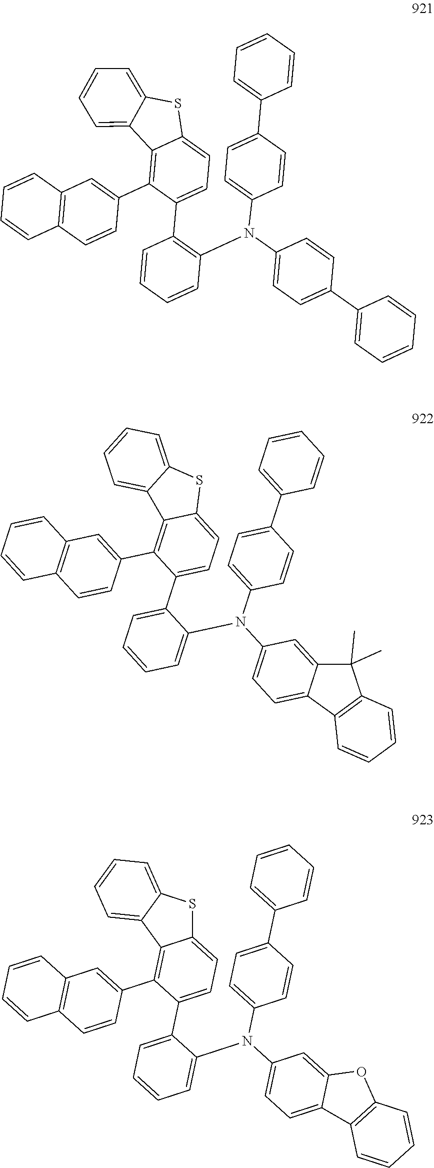 [SMILES: c1ccc(-c2ccc(N(c3ccc(-c4ccccc4)cc3)c3ccccc3-c3ccc4sc5ccccc5c4c3-c3ccc4ccccc4c3)cc2)cc1 ; CC1(C)c2ccccc2-c2ccc(N(c3ccc(-c4ccccc4)cc3)c3ccccc3-c3ccc4sc5ccccc5c4c3-c3ccc4ccccc4c3)cc21 ; c1ccc(-c2ccc(N(c3ccc4c(c3)oc3ccccc34)c3ccccc3-c3ccc4sc5ccccc5c4c3-c3ccc4ccccc4c3)cc2)cc1]

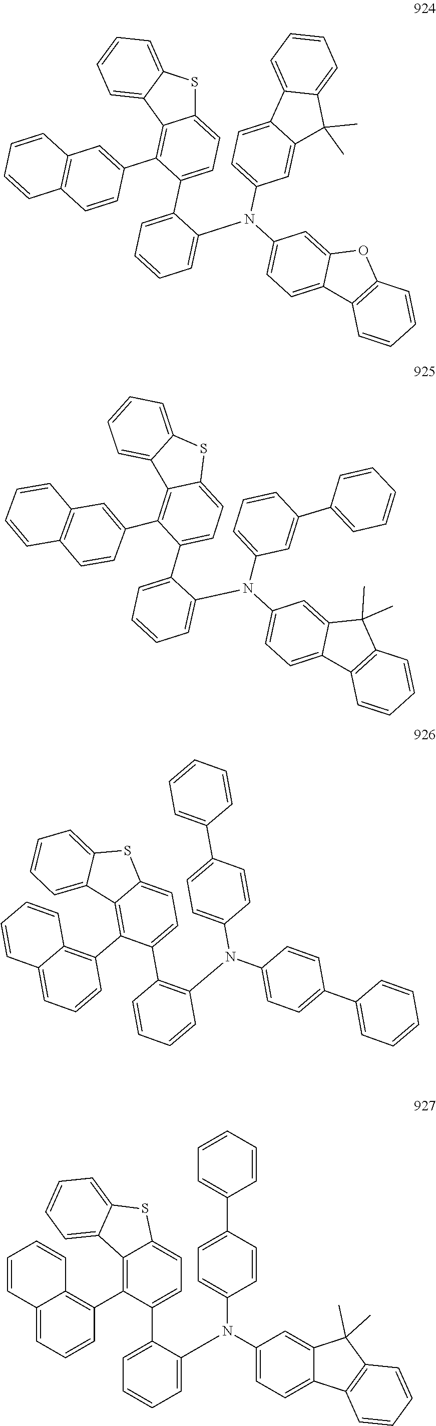 [SMILES: CC1(C)c2ccccc2-c2ccc(N(c3ccc4c(c3)oc3ccccc34)c3ccccc3-c3ccc4sc5ccccc5c4c3-c3ccc4ccccc4c3)cc21 ; CC1(C)c2ccccc2-c2ccc(N(c3cccc(-c4ccccc4)c3)c3ccccc3-c3ccc4sc5ccccc5c4c3-c3ccc4ccccc4c3)cc21 ; c1ccc(-c2ccc(N(c3ccc(-c4ccccc4)cc3)c3ccccc3-c3ccc4sc5ccccc5c4c3-c3cccc4ccccc34)cc2)cc1 ; CC1(C)c2ccccc2-c2ccc(N(c3ccc(-c4ccccc4)cc3)c3ccccc3-c3ccc4sc5ccccc5c4c3-c3cccc4ccccc34)cc21]

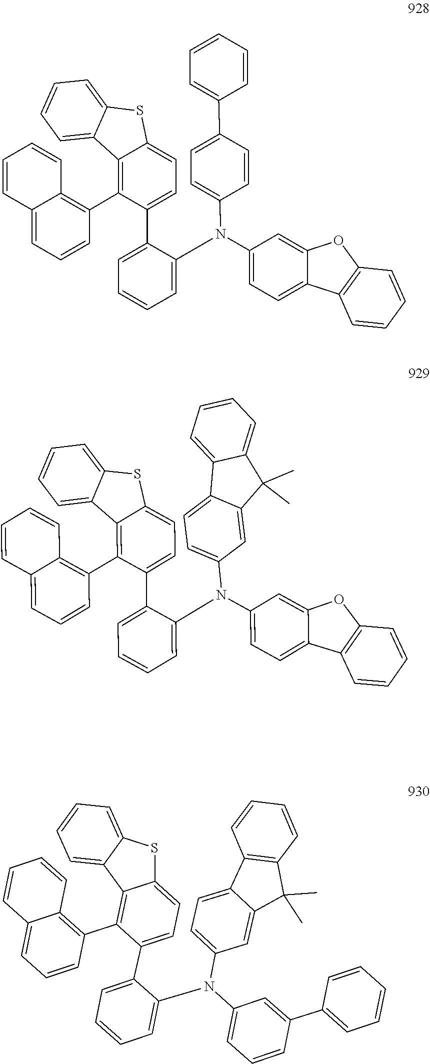 [SMILES: c1ccc(-c2ccc(N(c3ccc4c(c3)oc3ccccc34)c3ccccc3-c3ccc4sc5ccccc5c4c3-c3cccc4ccccc34)cc2)cc1 ; CC1(C)c2ccccc2-c2ccc(N(c3ccc4c(c3)oc3ccccc34)c3ccccc3-c3ccc4sc5ccccc5c4c3-c3cccc4ccccc34)cc21 ; CC1(C)c2ccccc2-c2ccc(N(c3cccc(-c4ccccc4)c3)c3ccccc3-c3ccc4sc5ccccc5c4c3-c3cccc4ccccc34)cc21]

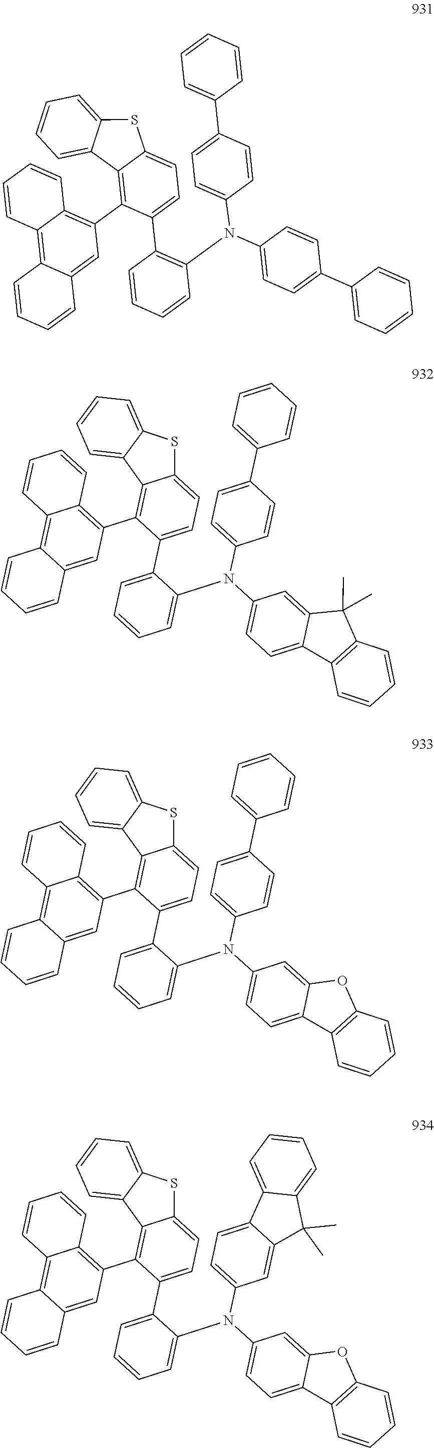 [SMILES: c1ccc(-c2ccc(N(c3ccc(-c4ccccc4)cc3)c3ccccc3-c3ccc4sc5ccccc5c4c3-c3cc4ccccc4c4ccccc34)cc2)cc1 ; CC1(C)c2ccccc2-c2ccc(N(c3ccc(-c4ccccc4)cc3)c3ccccc3-c3ccc4sc5ccccc5c4c3-c3cc4ccccc4c4ccccc34)cc21 ; c1ccc(-c2ccc(N(c3ccc4c(c3)oc3ccccc34)c3ccccc3-c3ccc4sc5ccccc5c4c3-c3cc4ccccc4c4ccccc34)cc2)cc1 ; CC1(C)c2ccccc2-c2ccc(N(c3ccc4c(c3)oc3ccccc34)c3ccccc3-c3ccc4sc5ccccc5c4c3-c3cc4ccccc4c4ccccc34)cc21]

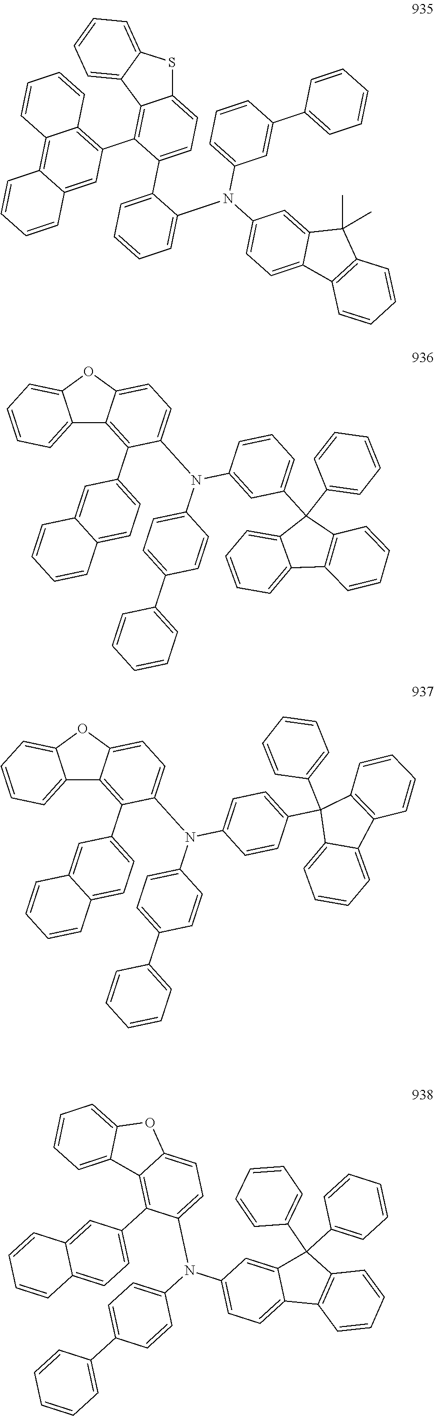 [SMILES: CC1(C)c2ccccc2-c2ccc(N(c3cccc(-c4ccccc4)c3)c3ccccc3-c3ccc4sc5ccccc5c4c3-c3cc4ccccc4c4ccccc34)cc21 ; c1ccc(-c2ccc(N(c3cccc(C4(c5ccccc5)c5ccccc5-c5ccccc54)c3)c3ccc4oc5ccccc5c4c3-c3ccc4ccccc4c3)cc2)cc1 ; c1ccc(-c2ccc(N(c3ccc(C4(c5ccccc5)c5ccccc5-c5ccccc54)cc3)c3ccc4oc5ccccc5c4c3-c3ccc4ccccc4c3)cc2)cc1 ; c1ccc(-c2ccc(N(c3ccc4c(c3)C(c3ccccc3)(c3ccccc3)c3ccccc3-4)c3ccc4oc5ccccc5c4c3-c3ccc4ccccc4c3)cc2)cc1]

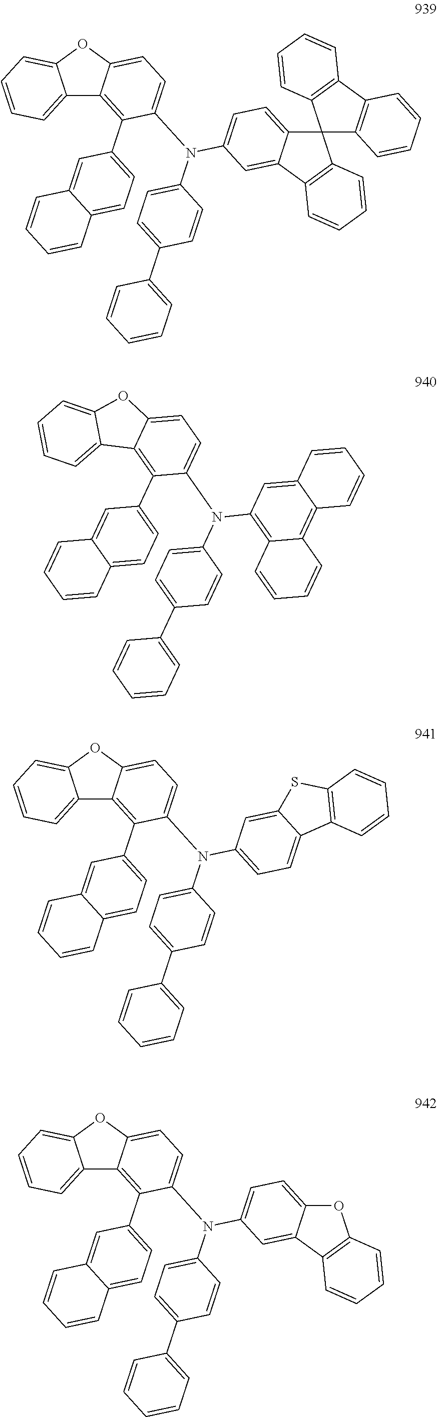 [SMILES: c1ccc(-c2ccc(N(c3ccc4c(c3)-c3ccccc3C43c4ccccc4-c4ccccc43)c3ccc4oc5ccccc5c4c3-c3ccc4ccccc4c3)cc2)cc1 ; c1ccc(-c2ccc(N(c3ccc4oc5ccccc5c4c3-c3ccc4ccccc4c3)c3cc4ccccc4c4ccccc34)cc2)cc1 ; c1ccc(-c2ccc(N(c3ccc4c(c3)sc3ccccc34)c3ccc4oc5ccccc5c4c3-c3ccc4ccccc4c3)cc2)cc1 ; c1ccc(-c2ccc(N(c3ccc4oc5ccccc5c4c3)c3ccc4oc5ccccc5c4c3-c3ccc4ccccc4c3)cc2)cc1]

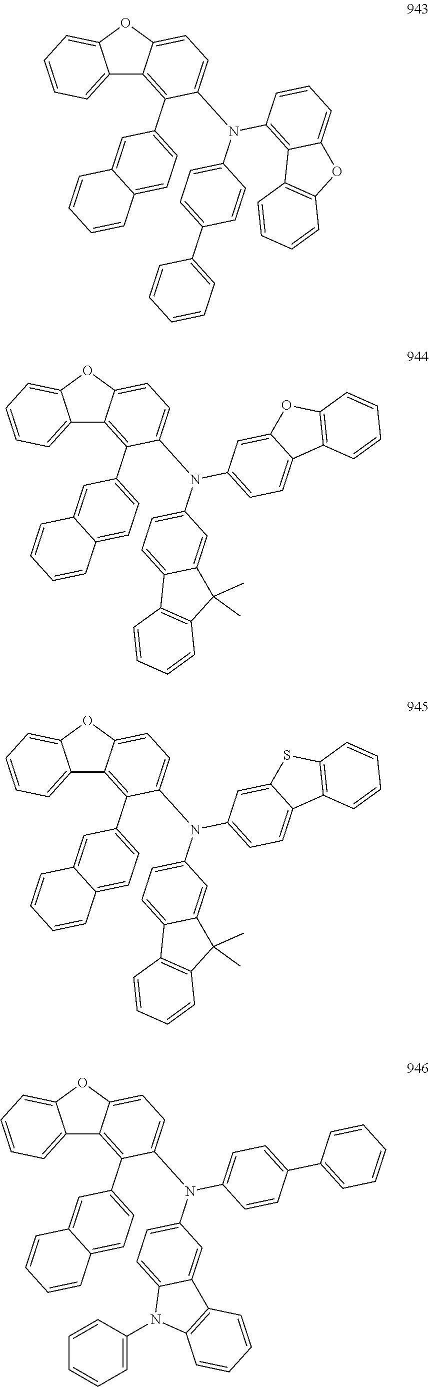 [SMILES: c1ccc(-c2ccc(N(c3ccc4oc5ccccc5c4c3-c3ccc4ccccc4c3)c3cccc4oc5ccccc5c34)cc2)cc1 ; CC1(C)c2ccccc2-c2ccc(N(c3ccc4c(c3)oc3ccccc34)c3ccc4oc5ccccc5c4c3-c3ccc4ccccc4c3)cc21 ; CC1(C)c2ccccc2-c2ccc(N(c3ccc4c(c3)sc3ccccc34)c3ccc4oc5ccccc5c4c3-c3ccc4ccccc4c3)cc21 ; c1ccc(-c2ccc(N(c3ccc4c(c3)c3ccccc3n4-c3ccccc3)c3ccc4oc5ccccc5c4c3-c3ccc4ccccc4c3)cc2)cc1]

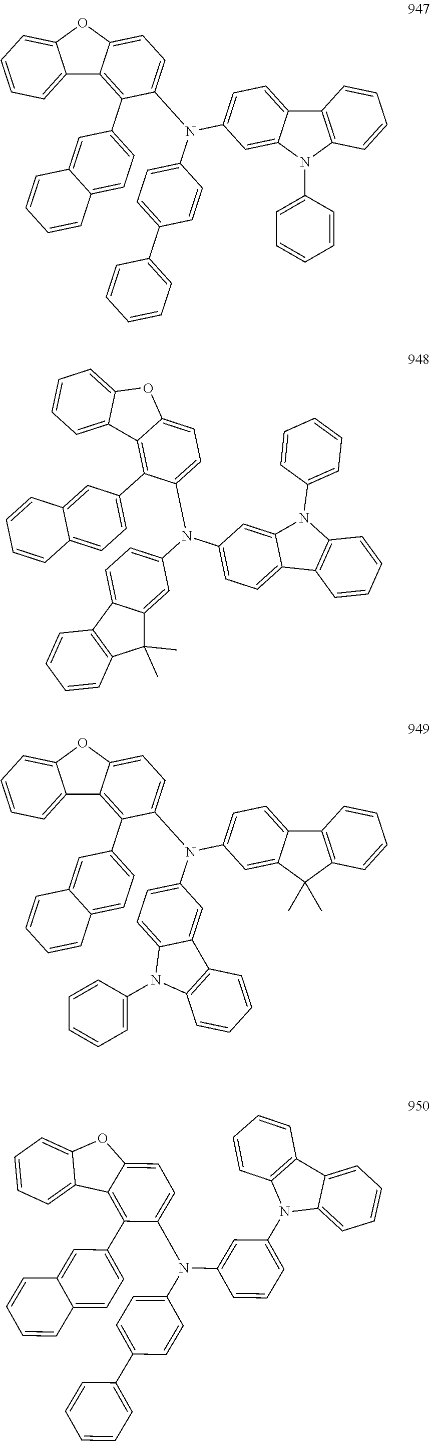 [SMILES: c1ccc(-c2ccc(N(c3ccc4c5ccccc5n(-c5ccccc5)c4c3)c3ccc4oc5ccccc5c4c3-c3ccc4ccccc4c3)cc2)cc1 ; CC1(C)c2ccccc2-c2ccc(N(c3ccc4c5ccccc5n(-c5ccccc5)c4c3)c3ccc4oc5ccccc5c4c3-c3ccc4ccccc4c3)cc21 ; CC1(C)c2ccccc2-c2ccc(N(c3ccc4c(c3)c3ccccc3n4-c3ccccc3)c3ccc4oc5ccccc5c4c3-c3ccc4ccccc4c3)cc21 ; c1ccc(-c2ccc(N(c3cccc(-n4c5ccccc5c5ccccc54)c3)c3ccc4oc5ccccc5c4c3-c3ccc4ccccc4c3)cc2)cc1]

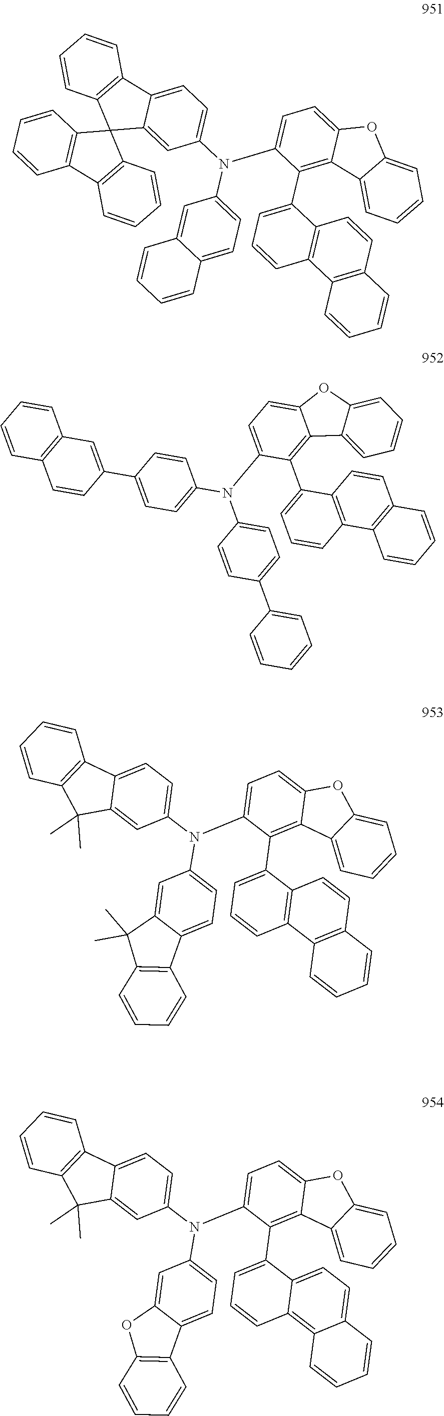 [SMILES: c1ccc2c(c1)-c1ccccc1C21c2ccccc2-c2ccc(N(c3ccc4ccccc4c3)c3ccc4oc5ccccc5c4c3-c3cccc4c3ccc3ccccc34)cc21 ; c1ccc(-c2ccc(N(c3ccc(-c4ccc5ccccc5c4)cc3)c3ccc4oc5ccccc5c4c3-c3cccc4c3ccc3ccccc34)cc2)cc1 ; CC1(C)c2ccccc2-c2ccc(N(c3ccc4c(c3)C(C)(C)c3ccccc3-4)c3ccc4oc5ccccc5c4c3-c3cccc4c3ccc3ccccc34)cc21 ; CC1(C)c2ccccc2-c2ccc(N(c3ccc4c(c3)oc3ccccc34)c3ccc4oc5ccccc5c4c3-c3cccc4c3ccc3ccccc34)cc21]

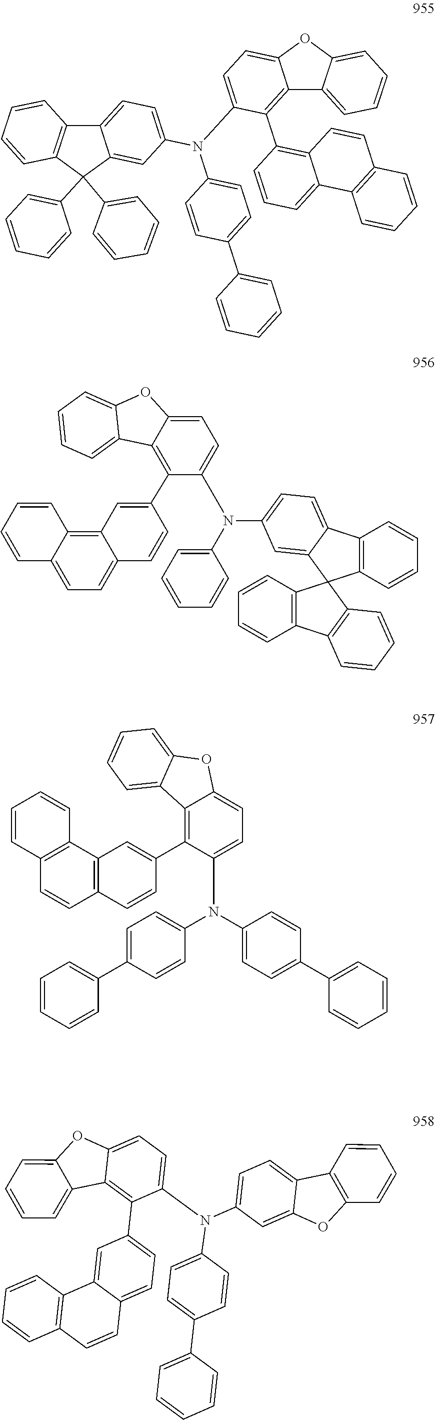 [SMILES: c1ccc(-c2ccc(N(c3ccc4c(c3)C(c3ccccc3)(c3ccccc3)c3ccccc3-4)c3ccc4oc5ccccc5c4c3-c3cccc4c3ccc3ccccc34)cc2)cc1 ; c1ccc(N(c2ccc3c(c2)C2(c4ccccc4-c4ccccc42)c2ccccc2-3)c2ccc3oc4ccccc4c3c2-c2ccc3ccc4ccccc4c3c2)cc1 ; c1ccc(-c2ccc(N(c3ccc(-c4ccccc4)cc3)c3ccc4oc5ccccc5c4c3-c3ccc4ccc5ccccc5c4c3)cc2)cc1 ; c1ccc(-c2ccc(N(c3ccc4c(c3)oc3ccccc34)c3ccc4oc5ccccc5c4c3-c3ccc4ccc5ccccc5c4c3)cc2)cc1]

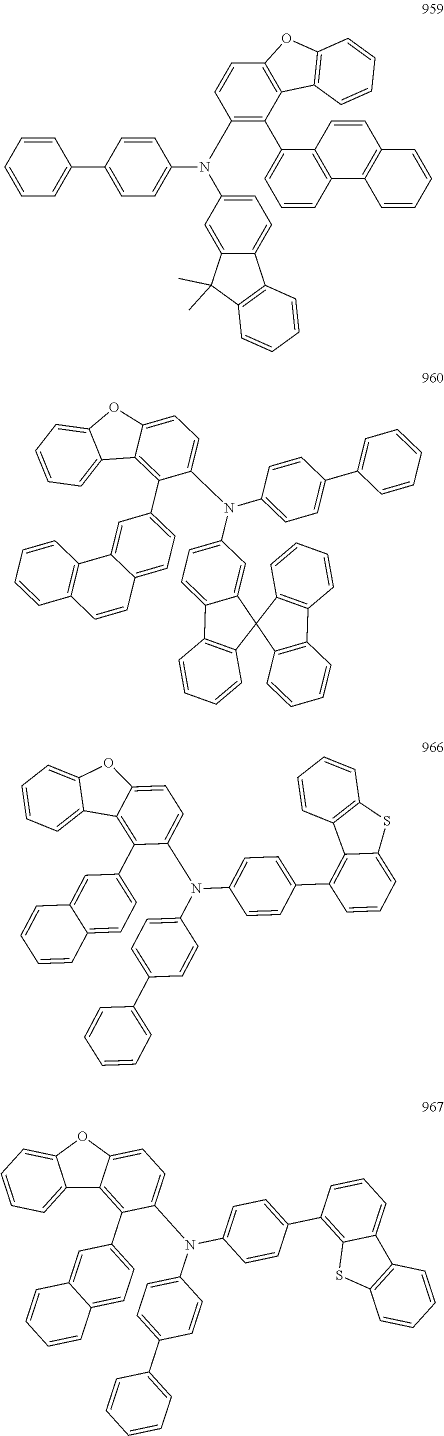 [SMILES: CC1(C)c2ccccc2-c2ccc(N(c3ccc(-c4ccccc4)cc3)c3ccc4oc5ccccc5c4c3-c3cccc4c3ccc3ccccc34)cc21 ; c1ccc(-c2ccc(N(c3ccc4c(c3)C3(c5ccccc5-c5ccccc53)c3ccccc3-4)c3ccc4oc5ccccc5c4c3-c3ccc4ccc5ccccc5c4c3)cc2)cc1 ; c1ccc(-c2ccc(N(c3ccc(-c4cccc5sc6ccccc6c45)cc3)c3ccc4oc5ccccc5c4c3-c3ccc4ccccc4c3)cc2)cc1 ; c1ccc(-c2ccc(N(c3ccc(-c4cccc5c4sc4ccccc45)cc3)c3ccc4oc5ccccc5c4c3-c3ccc4ccccc4c3)cc2)cc1]

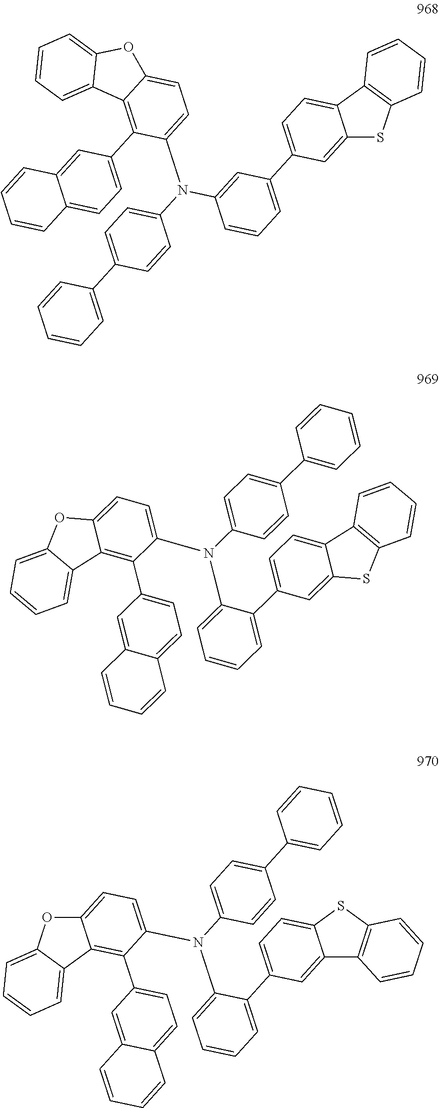 [SMILES: c1ccc(-c2ccc(N(c3cccc(-c4ccc5c(c4)sc4ccccc45)c3)c3ccc4oc5ccccc5c4c3-c3ccc4ccccc4c3)cc2)cc1 ; c1ccc(-c2ccc(N(c3ccccc3-c3ccc4c(c3)sc3ccccc34)c3ccc4oc5ccccc5c4c3-c3ccc4ccccc4c3)cc2)cc1 ; c1ccc(-c2ccc(N(c3ccccc3-c3ccc4sc5ccccc5c4c3)c3ccc4oc5ccccc5c4c3-c3ccc4ccccc4c3)cc2)cc1]

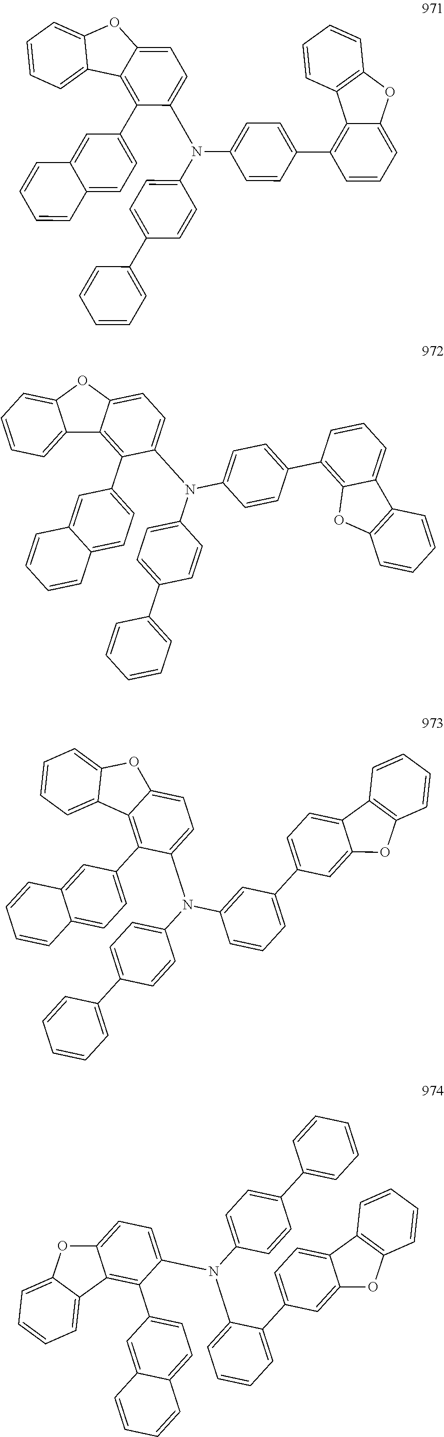 [SMILES: c1ccc(-c2ccc(N(c3ccc(-c4cccc5oc6ccccc6c45)cc3)c3ccc4oc5ccccc5c4c3-c3ccc4ccccc4c3)cc2)cc1 ; c1ccc(-c2ccc(N(c3ccc(-c4cccc5c4oc4ccccc45)cc3)c3ccc4oc5ccccc5c4c3-c3ccc4ccccc4c3)cc2)cc1 ; c1ccc(-c2ccc(N(c3cccc(-c4ccc5c(c4)oc4ccccc45)c3)c3ccc4oc5ccccc5c4c3-c3ccc4ccccc4c3)cc2)cc1 ; c1ccc(-c2ccc(N(c3ccccc3-c3ccc4c(c3)oc3ccccc34)c3ccc4oc5ccccc5c4c3-c3ccc4ccccc4c3)cc2)cc1]

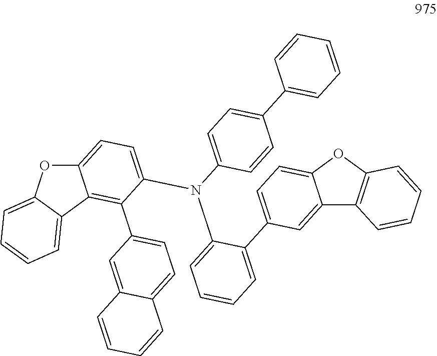 [SMILES: c1ccc(-c2ccc(N(c3ccccc3-c3ccc4oc5ccccc5c4c3)c3ccc4oc5ccccc5c4c3-c3ccc4ccccc4c3)cc2)cc1]

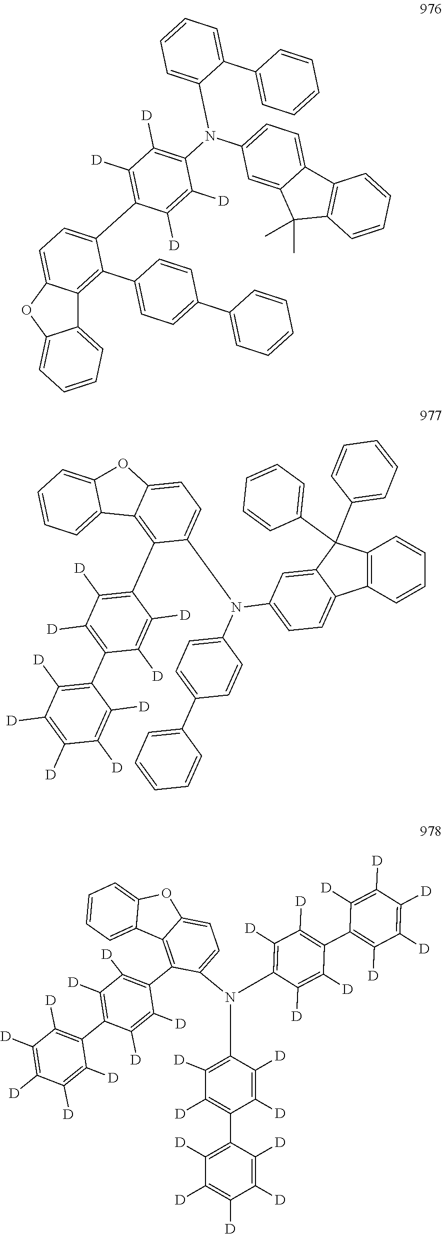

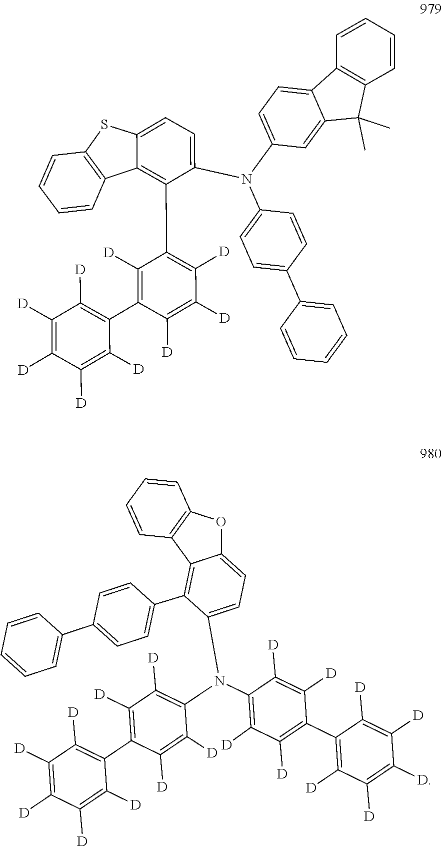

11. An organic electroluminescent device, comprising an anode and a cathode arranged oppositely, and a functional layer arranged between the anode and the cathode, wherein the functional layer contains the aromatic amine compound according to claim 1.

12. The organic electroluminescent device according to claim 11, wherein the functional layer comprises a hole auxiliary layer, and the hole auxiliary layer contains the aromatic amine compound.

13. An electronic apparatus, comprising the organic electroluminescent device according to claim 11.

14. The aromatic amine compound according to claim 2, wherein the substituent(s) in L₁ and L₂ are the same or different, and are each independently selected from deuterium, fluorine, cyano, methyl, ethyl, isopropyl, tert-butyl, trifluoromethyl, trideuteromethyl, and phenyl.

15. The aromatic amine compound according to claim 6, wherein the substituent(s) in Ar₁ and Ar₂ are the same or different, and are each independently selected from deuterium, fluorine, cyano, trideuteromethyl, trimethylsilyl, trifluoromethyl, cyclopentyl, cyclohexyl, adamantyl, methyl, ethyl, isopropyl, tert-butyl, phenyl, naphthyl, pyridyl, dibenzofuranyl, dibenzothienyl, and carbazolyl; optionally, any two adjacent substituents form a benzene ring or a fluorene ring.
